(19) United States
(12) Patent Application Publication
(10) Pub. No.: US 2026/0366001 A1
(43) Pub. Date: Nov. 19, 2026

(54) EVOLVED AND ENGINEERED PRIME EDITORS WITH IMPROVED EDITING EFFICIENCY

(71) Applicants: The Broad Institute, Inc., Cambridge, MA (US); President and Fellows of Harvard College, Cambridge, MA (US)

(72) Inventors: David R. Liu, Cambridge, MA (US); Jordan Leigh Doman, Cambridge, MA (US); Smriti Pandey, Cambridge, MA (US)

(73) Assignees: The Broad Institute, Inc., Cambridge, MA (US); President and Fellows of Harvard College, Cambridge, MA (US)

(21) Appl. No.: 19/486,804

(22) PCT Filed: May 23, 2024
(86) PCT No.: PCT/US2024/030786

§ 371 Date: Nov. 21, 2025

Related U.S. Application Data
(60) Provisional application No. 63/503,892, filed on May 23, 2023.
(60) Provisional application No. 63/506,026, filed on Jun. 2, 2023.
(60) Provisional application No. 63/508,616, filed on Jun. 16, 2023.
(60) Provisional application No. 63/510,078, filed on Jun. 23, 2023.
(60) Provisional application No. 63/596,006, filed on Nov. 3, 2023.

(51) Int. Cl. C12N 9/22; A61K 48/00; C12N 9/12; C12N 15/10; C12N 15/113; C12N 15/864

(52) U.S. Cl. CPC C12N 9/226; A61K 48/005; C12N 9/1276; C12N 15/102; C12N 15/113; C12N 15/8645; C12Y 207/07049; C12Y 301/26004; C12N 2310/20; C12N 2750/14143

(57) ABSTRACT

The present disclosure provides evolved and engineered reverse transcriptase variants and Cas9 variants with improved properties (e.g., improved editing efficiency when used in the context of a prime editor). Fusion proteins, including for example prime editors, comprising the reverse transcriptase variants and Cas9 variants described herein are also provided by the present disclosure. The present disclosure also provides polynucleotides encoding the reverse transcriptase variants, Cas9 variants, and prime editors provided herein, as well as vectors comprising such polynucleotides. Pharmaceutical compositions and cells comprising the reverse transcriptase variants, Cas9 variants, and prime editors described herein are also provided by the present disclosure. The present disclosure also provides methods and uses involving the reverse transcriptase variants, Cas9 variants, and prime editors described herein.

### RELATED APPLICATIONS

This application is a national stage filing under 35 U.S.C. § 371 of International PCT Application PCT/US2024/030786, filed May 23, 2024, which claims priority under 35 U.S.C. § 119(e) to U.S. Provisional application, U.S. Ser. No. 63/503,892, filed May 23, 2023; U.S. Provisional application, U.S. Ser. No. 63/506,026, filed Jun. 2, 2023; U.S. Provisional application, U.S. Ser. No. 63/510,078, filed Jun. 23, 2023; U.S. Provisional application, U.S. Ser. No. 63/596,006, filed Nov. 3, 2023; and U.S. Provisional application, U.S. Ser. No. 63/508,616, filed Jun. 16, 2023, each of which is incorporated herein by reference.

### GOVERNMENT SUPPORT

This invention was made with government support under Grant Nos. AI142756, AI150551, EB027793, GM118062, HG009490, and HL056067 awarded by the National Institutes of Health, and Grant No. DGE1144152 awarded by the National Science Foundation. The government has certain rights in the invention.

### REFERENCE TO AN ELECTRONIC SEQUENCE LISTING

The contents of the electronic sequence listing (B119570180US04-SEQ-GJM.xml; Size: 226,718 bytes; and Date of Creation: Nov. 20, 2025) are herein incorporated by reference in their entirety.

### BACKGROUND OF THE INVENTION

The ability to install precise, targeted changes into the genomes of living cells and organisms has advanced the understanding of biological systems and may provide one-time treatments for genetic diseases. Prime editing (PE) is a versatile gene editing technology capable of installing any base substitution, insertion, or deletion without generating DSBs¹. Since >95% of pathogenic substitutions, insertions, deletions, or combinations thereof are ≤50 bp in length², PE raises the possibility of correcting a large fraction of known disease-causing mutations. PE requires a prime editing guide RNA (pegRNA) and a prime editor protein, which comprises a programmable nickase (typically S. pyogenes Cas9 H840A nickase) and a reverse transcriptase (RT). The first-generation prime editor (PE1) used the wild-type Moloney murine leukemia virus (M-MLV) RT, while subsequent prime editors (PE2-PE5) use an engineered pentamutant M-MLV RT (FIG. 1A)^{1,3}. The pegRNA contains a core guide RNA scaffold that binds the programmable nickase, a spacer that specifies the target site, a primer binding site (PBS) that is complementary to the target DNA, and a reverse transcriptase template (RTT) that encodes the desired edit. To install an edit, the prime editor·pegRNA complex pairs with one strand of the target genomic locus and nicks the opposite strand to generate an exposed 3′ end of nicked genomic DNA, which binds to the complementary PBS of the pegRNA. The RT engages the resulting primer-template complex and initiates reverse transcription of the RTT, generating a 3′ DNA flap containing the desired edit. The newly synthesized 3′ flap is incorporated into the genome by cellular DNA repair pathways, replacing the original DNA sequence and leading to the permanent installation of the desired edit¹. In the PE3 and PE5 systems, an additional sgRNA is used to nick the non-edited DNA strand, improving editing efficiencies by biasing cellular mismatch repair to favor replacement of the non-edited strand (FIG. 1A)^{1,3}.

Since their development, PE systems have been improved by stabilizing or circularizing pegRNAs⁴⁻⁶, varying the prime editor architecture^{3,4}, and manipulating or evading cellular mismatch repair to favor desired editing outcomes^{3,9}. Twin prime editing (twinPE) and related methods have also been developed that use two pegRNAs to install edited sequence on both DNA strands, replacing the original genomic sequence between the two prime editing nicks with larger (>100-bp) programmable insertions and deletions^{10-13,15,16}. Prime editing and twinPE have also been used to install site-specific recombinase landing sites, enabling recombinase-mediated gene-sized (>5,000 bp) targeted insertions or inversions¹⁰. Prime editing, twin prime editing, and prime editors are further described, e.g., in International Patent Application No. PCT/US2020/023721, filed Mar. 19, 2020, which published as WO 2020/191239; International Patent Application No. PCT/US2021/031439, filed May 7, 2021, which published as WO 2021/226558; and International Patent Application No. PCT/2021/052097, filed Sep. 24, 2021, which published as WO 2022/067130; the contents of each of which is incorporated by reference herein.

Despite this progress, the reverse transcriptase at the heart of prime editors has proven challenging to improve through protein engineering. Many of the prime editing systems reported to date, including the current PE4max and PE5max systems, use the engineered M-MLV RT in PE2. The five M-MLV RT mutations in PE2 were identified over several decades of in vitro screening for improved RT variants¹⁸⁻²¹, followed by screening of many combinations of M-MLV RT mutants that optimize prime editing efficiencies¹. While these mutations are critical to the efficiency of prime editing, few analogous mutations have been described for other RTs that have been tested in prime editing experiments. Prime editor proteins that use non-M-MLV RTs in principle could offer important benefits, including smaller size that could facilitate in vivo prime editor delivery, mRNA production, or ribonucleoprotein (RNP) preparation. Different RT enzymes may also improve properties of PE such as editing efficiency, suitability for longer or shorter prime edits, or compatibility with installing sequences of different composition, just as different deaminases have provided a diverse collection of base editors that greatly increase the likelihood of finding one ideally suited to a particular application²². Despite these potential benefits, all previously reported prime editors that do not use the engineered M-MLV RT in PE2 have shown substantially lower prime editing efficiencies than PE2 for most target sequences, even after extensive protein engineering^{4,17,24}. Further improvement of the highly engineered M-MLV RT in PE2 has also proven difficult, as all reported variants of this RT have also yielded little or no improvements in prime editing efficiency in mammalian cells^{17,24}. Similarly, although it has been shown that Cas9 mutations known to improve nuclease performance can also increase prime editing efficiency³, mutants of Cas9 identified specifically to improve prime editing have not yet been reported. Accordingly, additional RT and Cas9 variants evolved and/or engineered with the purpose of improving prime editing efficiency would advance the art.

### SUMMARY OF THE INVENTION

As presented herein, a phage-assisted continuous evolution (PACE)²⁶ selection for prime editing was developed, and PE PACE and protein engineering was used to generate new polymerase (e.g., reverse transcriptase or “RT”) and Cas9 variants that enhance prime editing efficiency and in-vivo deliverability. First, natural RTs were screened from a wide variety of organisms, and it was found that most exhibited negligible prime editing activity in mammalian cells. Two weakly active RTs, those from the Escherichia coli Ec48 retron²⁷ and from the Schizosaccharomyces pombe Tf1 retrotransposon²⁸, were evolved to create next-generation prime editors (PE6a and PE6b) that are 516-810 bp smaller than PE2 while offering mammalian prime editing efficiencies comparable to or higher than those of PE2 for many target sites and types of edits. It was discovered that the reduced RT processivity of PEmaxΔRNaseH (i.e., PEmax comprising an MMLV reverse transcriptase with a truncation of the C-terminal RNaseH domain), the commonly used prime editor variant used in dual-AAV delivery systems^{4,23,29-31}, causes it to underperform at long edits with a high degree of secondary structure. To generate dual AAV-compatible RTs that can install longer edits or edits that require RT templates with a high degree of secondary structure, PE PACE and protein engineering were used to generate PE6c and PE6d. These RT mutants offer large benefits in editing efficiency compared to PEmaxΔRNaseH for edits that require structured pegRNA RT templates. The PE6a-PE6d RTs also offer improvements in editing efficiency and fewer indel frequencies over full-length PEmax. Finally, PE PACE was used to evolve the Cas9 nickase domain of prime editors to create PE6e-PE6g, which further improve prime editing efficiencies. The improved RT and Cas9 nickase domains of PE6 variants can be combined with each other, as well as with mismatch repair evasion strategies³, epegRNAs⁵, and the PEmax architecture³, to offer cumulative benefits in a variety of contexts, including in patient-derived fibroblasts and primary human T cells. Finally, it is demonstrated that PE6c and PE6d are uniquely enabling for performing long prime edits and twinPE in vivo. After dual-AAV delivery of PE6 systems, on average 12- to 183-fold improvement in prime editing efficiency was achieved compared with previous state-of-the-art systems for installation of 38- to 42-bp edits in the mouse cortex, yielding 62% targeted installation of the loxP sequence among transduced cells in the mouse cortex.

The improved prime editors PE6a-PE6g described herein comprise the following amino acid substitutions relative to particular wild-type reverse transcriptase or Cas9 proteins:

| | | Amino Acid Substitutions | |
|---|---|---|---|
| Prime | Engineered | Compared to Reference | Reference |
| Editor | Component | Sequence | Sequence |
| PE6a | Reverse | E60K, K87E, E165D, D243N, | Ec48 reverse |
| | Transcriptase | R267I, E279K, K318E, and | transcriptase |
| | | K343N | (SEQ ID NO: 7) |
| PE6b | Reverse | P70T, G72V, S87G, M102I, | Tf1 reverse |
| | Transcriptase | K106R, K118R, I128V, L158Q, | transcriptase |
| | | F269L, A363V, K413E, and | (SEQ ID NO: 1) |
| | | S492N | |
| PE6c | Reverse | P70T, G72V, S87G, M102I, | Tf1 reverse |
| | Transcriptase | K106R, K118R, I128V, L158Q, | transcriptase |
| | | S188K, I260L, F269L, R288Q, | (SEQ ID NO: 1) |
| | | S297Q, A363V, K413E, and | |
| | | S492N | |
| PE6d | Reverse | T128N, D200C, and V223Y (and | MMLV reverse |
| | Transcriptase | the substitutions T306K, W313F, | transcriptase |
| | | and T330P used in the MMLV | (SEQ ID NO: 30) |
| | | reverse transcriptase of PE2 | with RNaseH domain |
| | | and PEmax) | truncation (e.g., |
| | | | truncation between |
| | | | D497 and I498 of |
| | | | SEQ ID NO: 30) |
| PE6e | Cas9 | K775R and K918A | Streptococcus |
| | | | pyogenes Cas9 |
| | | | nickase |
| | | | (SEQ ID NO: 2) |
| PE6f | Cas9 | H99R, E471K, I632V, | Streptococcus |
| | | D645N, H721Y, and K918A | pyogenes Cas9 |
| | | | nickase |
| | | | (SEQ ID NO: 2) |
| PE6g | Cas9 | H99R, E471K, I632V, | Streptococcus |
| | | D645N, R654C, and H721Y | pyogenes Cas9 |
| | | | nickase |
| | | | (SEQ ID NO: 2) |

In some embodiments, the present disclosure provides prime editors comprising the reverse transcriptase of PE6a (or a reverse transcriptase at least 80%, at least 85%, at least 90%, at least 95%, at least 96%, at least 97%, at least 98%, or at least 99% identical to the reverse transcriptase of PE6a) and a nucleic acid-programmable DNA-binding protein (napDNAbp) (e.g., a Cas9 protein). In some embodiments, the present disclosure provides prime editors comprising the reverse transcriptase of PE6b (or a reverse transcriptase at least 80%, at least 85%, at least 90%, at least 95%, at least 96%, at least 97%, at least 98%, or at least 99% identical to the reverse transcriptase of PE6b) and a napDNAbp (e.g., a Cas9 protein). In some embodiments, the present disclosure provides prime editors comprising the reverse transcriptase of PE6c (or a reverse transcriptase at least 80%, at least 85%, at least 90%, at least 95%, at least 96%, at least 97%, at least 98%, or at least 99% identical to the reverse transcriptase of PE6c) and a napDNAbp (e.g., a Cas9 protein). In some embodiments, the present disclosure provides prime editors comprising the reverse transcriptase of PE6d (or a reverse transcriptase at least 80%, at least 85%, at least 90%, at least 95%, at least 96%, at least 97%, at least 98%, or at least 99% identical to the reverse transcriptase of PE6d) and a napDNAbp (e.g., a Cas9 protein and a napDNAbp (e.g., a Cas9 protein).

In some embodiments, the present disclosure provides prime editors comprising the Cas9 protein of PE6e (or a Cas9 protein at least 80%, at least 85%, at least 90%, at least 95%, at least 96%, at least 97%, at least 98%, or at least 99% identical to the Cas9 protein of PE6e) and a polymerase (e.g., a reverse transcriptase). In some embodiments, the present disclosure provides prime editors comprising the Cas9 protein of PE6f (or a Cas9 protein at least 80%, at least 85%, at least 90%, at least 95%, at least 96%, at least 97%, at least 98%, or at least 99% identical to the Cas9 protein of PE6f) and a polymerase (e.g., a reverse transcriptase). In some embodiments, the present disclosure provides prime editors comprising the Cas9 protein of PE6g (or a Cas9 protein at least 80%, at least 85%, at least 90%, at least 95%, at least 96%, at least 97%, at least 98%, or at least 99% identical to the Cas9 protein of PE6g) and a polymerase (e.g., a reverse transcriptase).

In certain embodiments, the present disclosure provides prime editors comprising the reverse transcriptase of PE6a and the Cas9 protein of PE6e (PE6a-e). In certain embodiments, the present disclosure provides prime editors comprising the reverse transcriptase of PE6a and the Cas9 protein of PE6f (PE6a-f). In certain embodiments, the present disclosure provides prime editors comprising the reverse transcriptase of PE6a and the Cas9 protein of PE6g (PE6a-g). In certain embodiments, the present disclosure provides prime editors comprising the reverse transcriptase of PE6b and the Cas9 protein of PE6e (PE6b-e). In certain embodiments, the present disclosure provides prime editors comprising the reverse transcriptase of PE6b and the Cas9 protein of PE6f (PE6b-f). In certain embodiments, the present disclosure provides prime editors comprising the reverse transcriptase of PE6b and the Cas9 protein of PE6g (PE6b-g). In certain embodiments, the present disclosure provides prime editors comprising the reverse transcriptase of PE6c and the Cas9 protein of PE6e (PE6c-e). In certain embodiments, the present disclosure provides prime editors comprising the reverse transcriptase of PE6c and the Cas9 protein of PE6f (PE6c-f). In certain embodiments, the present disclosure provides prime editors comprising the reverse transcriptase of PE6c and the Cas9 protein of PE6g (PE6c-g). In certain embodiments, the present disclosure provides prime editors comprising the reverse transcriptase of PE6d and the Cas9 protein of PE6e (PE6d-e). In certain embodiments, the present disclosure provides prime editors comprising the reverse transcriptase of PE6d and the Cas9 protein of PE6f (PE6d-f). In certain embodiments, the present disclosure provides prime editors comprising the reverse transcriptase of PE6d and the Cas9 protein of PE6g (PE6d-g).

In one aspect, the present disclosure provides reverse transcriptase variants having at least 80%, at least 85%, at least 90%, at least 95%, at least 96%, at least 97%, at least 98%, or at least 99% sequence identity with SEQ ID NO: 1 (Tf1 reverse transcriptase), wherein the reverse transcriptase variant comprises amino acid substitutions at positions 70, 72, 87, 102, 106, 118, 128, 158, 269, 363, 413, and 492 relative to SEQ ID NO: 1, or corresponding substitutions in a homologous sequence. In some embodiments, the reverse transcriptase variant further comprises amino acid substitutions at positions 188, 260, 297, and 288 relative to SEQ ID NO: 1.

In another aspect, the present disclosure provides reverse transcriptase variants having at least 80%, at least 85%, at least 90%, at least 95%, at least 96%, at least 97%, at least 98%, or at least 99% sequence identity with SEQ ID NO: 30 (MMLV reverse transcriptase), wherein the reverse transcriptase variant comprises amino acid substitutions at positions 128 and 200 relative to SEQ ID NO: 30, or corresponding substitutions in a homologous sequence. In some embodiments, the reverse transcriptase variant further comprises amino acid substitutions at positions 223, 306, 313, and 330 relative to SEQ ID NO: 30, or corresponding substitutions in a homologous sequence. In some embodiments, the reverse transcriptase variant comprises a truncation of the RNaseH domain of SEQ ID NO: 30 (e.g., a truncation at D497 in SEQ ID NO: 30).

In another aspect, the present disclosure provides reverse transcriptase variants having at least 80%, at least 85%, at least 90%, at least 95%, at least 96%, at least 97%, at least 98%, or at least 99% sequence identity with SEQ ID NO: 30 (MMLV reverse transcriptase), wherein the reverse transcriptase variant comprises the amino acid substitutions T128N and V223M; T128N and V223Y; T128F and V223M; or D200C and V223M relative to SEQ ID NO: 30, or corresponding substitutions in a homologous sequence.

In another aspect, the present disclosure provides reverse transcriptase variants having at least 80%, at least 85%, at least 90%, at least 95%, at least 96%, at least 97%, at least 98%, or at least 99% sequence identity with SEQ ID NO: 30 (MMLV reverse transcriptase), wherein the reverse transcriptase variant comprises amino acid substitutions at positions 128, 129, 196, 200, and 223 relative to SEQ ID NO: 30, or corresponding substitutions in a homologous sequence.

In another aspect, the present disclosure provides Cas9 variants having at least 80%, at least 85%, at least 90%, at least 95%, at least 96%, at least 97%, at least 98%, or at least 99% sequence identity with SEQ ID NO: 2 (Streptococcus pyogenes Cas9 nickase), wherein the Cas9 variant comprises amino acid substitutions at positions 775 and 918 relative to SEQ ID NO: 2, or corresponding substitutions in a homologous sequence.

In another aspect, the present disclosure provides Cas9 variants having at least 80%, at least 85%, at least 90%, at least 95%, at least 96%, at least 97%, at least 98%, or at least 99% sequence identity with SEQ ID NO: 2 (Streptococcus pyogenes Cas9 nickase), wherein the Cas9 variant comprises amino acid substitutions at positions 99, 471, 632, 645, and 721 relative to SEQ ID NO: 2, or corresponding substitutions in a homologous sequence.

In another aspect, the present disclosure provides Cas9 variants having at least 80%, at least 85%, at least 90%, at least 95%, at least 96%, at least 97%, at least 98%, or at least 99% sequence identity with SEQ ID NO: 2 (Streptococcus pyogenes Cas9 nickase), wherein the Cas9 variant comprises amino acid substitutions at positions 99, 471, and 632 relative to SEQ ID NO: 2, or corresponding substitutions in a homologous sequence.

In another aspect, the present disclosure provides Cas9 variants having at least 80%, at least 85%, at least 90%, at least 95%, at least 96%, at least 97%, at least 98%, or at least 99% sequence identity with SEQ ID NO: 2 (Streptococcus pyogenes Cas9 nickase), wherein the Cas9 variant comprises amino acid substitutions at positions 471 and 918 relative to SEQ ID NO: 2, or corresponding substitutions in a homologous sequence.

In another aspect, the present disclosure provides Cas9 variants having at least 80%, at least 85%, at least 90%, at least 95%, at least 96%, at least 97%, at least 98%, or at least 99% sequence identity with SEQ ID NO: 2 (Streptococcus pyogenes Cas9 nickase), wherein the Cas9 variant comprises amino acid substitutions at positions 753 and 1151 relative to SEQ ID NO: 2, or corresponding substitutions in a homologous sequence.

In another aspect, the present disclosure provides Cas9 variants having at least 80%, at least 85%, at least 90%, at least 95%, at least 96%, at least 97%, at least 98%, or at least 99% sequence identity with SEQ ID NO: 2 (Streptococcus pyogenes Cas9 nickase), wherein the Cas9 variant comprises one or more amino acid substitutions at positions selected from the group consisting of 260, 298, 395, 769, 778, 1014, 1034, 1100, 1106, 1138, 1152, and 1320 relative to SEQ ID NO: 2, or corresponding substitutions in a homologous sequence.

In another aspect, the present disclosure provides Cas9 variants having at least 80%, at least 85%, at least 90%, at least 95%, at least 96%, at least 97%, at least 98%, or at least 99% sequence identity with SEQ ID NO: 2 (Streptococcus pyogenes Cas9 nickase), wherein the Cas9 variant comprises amino acid substitutions at positions 23 and 754 relative to SEQ ID NO: 2, or corresponding substitutions in a homologous sequence.

In another aspect, the present disclosure provides prime editors comprising (i) any of the reverse transcriptase variants provided herein, and (ii) a napDNAbp, for example, a Cas9 protein (e.g., a Cas9 nickase, or any of the Cas9 variants provided herein (which may also be Cas9 nickases), or a Cas9 nuclease or nuclease-inactivated Cas9 (dCas9)).

In another aspect, the present disclosure provides prime editors comprising (i) any of the Cas9 variants provided herein, and (ii) a polymerase (e.g., a reverse transcriptase, such as any of the reverse transcriptase variants provided herein). In some embodiments, the reverse transcriptase comprises a sequence having at least 80%, at least 85%, at least 90%, at least 95%, at least 96%, at least 97%, at least 98%, or at least 99% sequence identity with SEQ ID NO: 7 (Ec48 reverse transcriptase), wherein the reverse transcriptase comprises amino acid substitutions at positions 60, 87, 165, 243, 267, 279, 318, and 343 relative to SEQ ID NO: 7, or corresponding positions in a homologous sequence.

In some embodiments, the reverse transcriptase variants provided herein comprise the amino acid sequence of any one of SEQ ID NOs: 25-27 or 50, or an amino acid sequence at least 80%, at least 85%, at least 90%, at least 95%, at least 96%, at least 97%, at least 98%, or at least 99% identical to the amino acid sequence of any one of SEQ ID NOs: 25-27 or 50. In some embodiments, the Cas9 variants provided herein comprise the amino acid sequence of any one of SEQ ID NOs: 28, 48, or 49, or an amino acid sequence at least 80%, at least 85%, at least 90%, at least 95%, at least 96%, at least 97%, at least 98%, or at least 99% identical to the amino acid sequence of any one of SEQ ID NOs: 28, 48, or 49. In some embodiments, the prime editors provided herein comprise a Cas9 variant comprising the amino acid sequence of any one of SEQ ID NOs: 28, 48, or 49, or an amino acid sequence at least 80%, at least 85%, at least 90%, at least 95%, at least 96%, at least 97%, at least 98%, or at least 99% identical to the amino acid sequence of any one of SEQ ID NOs: 28, 48, or 49, and a reverse transcriptase variant comprising the amino acid sequence of any one of SEQ ID NOs: 25-27 or 50, or an amino acid sequence at least 80%, at least 85%, at least 90%, at least 95%, at least 96%, at least 97%, at least 98%, or at least 99% identical to the amino acid sequence of any one of SEQ ID NOs: 25-27 or 50.

In another aspect, the present disclosure provides fusion proteins comprising any of the Cas9 variants provided herein and an effector domain. In certain embodiments, the effector domain comprises nuclease activity, nickase activity, recombinase activity, deaminase activity, methyltransferase activity, methylase activity, acetylase activity, acetyltransferase activity, transcriptional activation activity, transcriptional repression activity, or polymerase activity.

In another aspect, the present disclosure provides complexes comprising any of the prime editors or other fusion proteins provided herein and a prime editing guide RNA (pegRNA).

In some aspects, the present disclosure provides polynucleotides encoding any of the reverse transcriptase variants, Cas9 variants, fusion proteins, or prime editors provided herein. In another aspect, the present disclosure provides vectors comprising any of the polynucleotides provided herein.

In another aspect, the present disclosure provides adeno-associated virus (AAV) particles comprising any of the reverse transcriptase variants, Cas9 variants, prime editors, fusion proteins, complexes, polynucleotides, and/or vectors provided herein.

In another aspect, the present disclosure provides cells comprising any of the reverse transcriptase variants, Cas9 variants, prime editors, fusion proteins, complexes, polynucleotides, vectors, and/or AAV particles provided herein.

In another aspect, the present disclosure provides pharmaceutical compositions comprising any of the reverse transcriptase variants, Cas9 variants, prime editors, fusion proteins, complexes, polynucleotides, vectors, AAV particles, and/or cells provided herein.

In another aspect, the present disclosure provides methods for editing a nucleic acid molecule by prime editing comprising contacting a nucleic acid molecule with any of the prime editors or complexes provided herein. In certain embodiments, the edit in the nucleic acid molecule comprises one or more nucleotide insertions, one or more nucleotide substitutions, one or more nucleotide deletions, or a combination thereof. In certain embodiments, the method is a method of twin prime editing (also known as dual flap prime editing). In some embodiments, the present disclosure provides methods of using the prime editors, complexes, polynucleotides, or vectors provided herein in veterinary uses. In some embodiments, the present disclosure provides methods of using the prime editors, complexes, polynucleotides, or vectors provided herein in agricultural uses.

In another aspect, the present disclosure provides kits comprising any of the reverse transcriptase variants, Cas9 variants, prime editors, fusion proteins, complexes, polynucleotides, vectors, AAV particles, and/or cells provided herein.

In another aspect, the present disclosure provides for the use of any of the reverse transcriptase variants, Cas9 variants, prime editors, fusion proteins, complexes, polynucleotides, vectors, AAV particles, and/or cells provided herein in the manufacture of a medicament.

In another aspect, the present disclosure provides for the use of any of the reverse transcriptase variants, Cas9 variants, prime editors, fusion proteins, complexes, polynucleotides, vectors, AAV particles, and/or cells provided herein in medicine.

In some aspects, the present disclosure provides systems for phage-assisted continuous and non-continuous evolution (PACE and PANCE) of prime editors. In certain embodiments, the present disclosure provides systems comprising: i) a first polynucleotide encoding a pegRNA and the gIII gene; ii) a second polynucleotide encoding a Cas9 protein fused to an N-intein; iii) a third polynucleotide encoding an RNA polymerase; iv) a fourth polynucleotide encoding proteins capable of mutagenizing a phage, optionally wherein the fourth polynucleotide comprises the MP6 plasmid; and v) a fifth polynucleotide encoding a reverse transcriptase fused to a C-intein. In certain embodiments, the present disclosure provides systems comprising i) a first polynucleotide encoding a pegRNA and the gIII gene; ii) a second polynucleotide encoding a prime editor; iii) a third polynucleotide encoding an RNA polymerase; and iv) a fourth polynucleotide encoding proteins capable of mutagenizing a phage, optionally wherein the fourth polynucleotide comprises the MP6 plasmid.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying Figures.

### BRIEF DESCRIPTION OF THE DRAWINGS

The following Figures form part of the present specification and are included to further demonstrate certain aspects of the present disclosure, which can be better understood by reference to one or more of these drawings in combination with the detailed description of specific embodiments presented herein.

FIGS. 1A-1J: Identification and engineering of reverse transcriptase enzymes into new prime editor candidates. FIG. 1A shows an overview of prime editing using the PE1, PE2, and PE3 systems. All three systems use a prime editor protein comprising SpCas9(H840A) nickase fused to a reverse transcriptase (RT) enzyme. The PE1 system uses the RT from the Moloney murine leukemia virus (M-MLV), while the PE2 system uses an engineered pentamutant variant of the M-MLV RT with D200N, L603W, T306K, W313F, and T330P. An additional single guide RNA (sgRNA) is used in the PE3 system to nick the non-edited strand. PBS=primer binding site. RT template=reverse transcriptase template. FIG. 1B shows phylogenetic classification of all RTs tested for prime editing herein (circles). Enzymes that exhibit activity in the PE system (dark gray circles) belong to four different RT classes. FIG. 1C shows 20 different RT enzymes other than the M-MLV RT exhibit activity in the prime editing system at endogenous sites in HEK293T cells. Bars reflect the mean of n=3 independent replicates. Dots show individual replicate values. Throughout all figures, prime editing efficiencies shown reflect the frequency of the intended prime editing outcome with no indels or other changes at the target site. FIG. 1D shows comparison of wild type (WT) Tf1 RT, PE2ΔRNaseH (i.e., comprising a truncation of the C-terminal RNaseH domain of the MMLV reverse transcriptase, e.g., between amino acids D497 and I498 in an MMLV reverse transcriptase of SEQ ID NO: 30), and PE2 at three longer, complex PE (HEK3) or twinPE (CCR5 and IDS) edits in HEK293T cells. Bars reflect the mean of n=3 independent replicates. Dots show individual replicate values. FIG. 1E shows a comparison of prime editors containing engineered retroviral RT variants with their WT counterparts in HEK293T cells. rdPERV=porcine endogenous retrovirus RT D200N, T306K, W313F, E330P, L603W. rdAVIRE=avian reticuloendotheliosis virus RT D200N, T306K, W313F, G330P, L603W. rdKORV=koala retrovirus RT D198N, T304K, W311F, E328P, L600W. rdWMSV=woolly monkey sarcoma virus RT D198N, T304K, W311F, E328P, L600W. All values from n=3 independent replicates are shown. Horizontal bars show the mean value. FIG. 1F shows residues mutated to improve editing of the Tf1 RT prime editor correspond to V188, R118, L258, M281 and V286 (red) in Ty3 RT (blue). V188 and R118 are in close proximity to the RNA (green) substrate and correspond to K118 and S188 in Tf1, respectively. L258, M281 and V286 are near the DNA (yellow) substrate and correspond to I260, S297 and R288 in Tf1, respectively. FIG. 1G shows that rationally designed Tf1 pentamutant variant (rdTf1) shows improvements in editing over its WT counterpart in HEK293T cells. Bars reflect the mean of n=3 independent replicates. Dots show individual replicate values. All edits are PE edits, except the AAVS1 site, which is twinPE. rdTf1=Tf1 RT K118R, S188K, I260L, S297Q, R288Q. FIG. 1H shows that rationally designed Ec48 triple mutant variant (rdEc48) shows improvements in editing over its WT counterpart for five edits in HEK293T cells. Bars reflect the mean of n=3 independent replicates. Dots show individual replicate values. rdEc48=Ec48 RT R315K, L182N, T189N. FIG. 1I shows a comparison of prime editors containing engineered RT variants with PE2 in HEK293T cells. All values from n=3 independent replicates are shown. Horizontal bars show the mean value. All edits using single-flap prime editing, except the AAVS1 site, which uses twinPE. FIG. 1J shows a comparison of rdTf1 with PE2 and its WT counterpart at three longer, complex PE (HEK3), or twinPE (CCR5 and IDS) edits in HEK293T cells. Bars reflect the mean of n=3 independent replicates. Dots show individual replicate values.

FIGS. 2A-2K: Development and validation of a prime editing PACE selection. FIG. 2A is a schematic of PE PACE selection circuit. Upon infection of host E. coli cells by selection phage (SP, blue), the NpuN intein and NpuC intein (pink) mediate protein splicing to reconstitute the two halves of the PE2 prime editor (purple and pink). The prime editor then engages a pegRNA (dark green) and corrects a frameshift in T7 RNAP (orange) via prime editing. Functional T7 RNAP then transcribes gIII (light green), which enables propagation of the SP. FIG. 2B shows evaluation of the v1 PE PACE circuit. Phage replication levels from overnight propagation of empty phage (red), NpuC-PE2-RT phage (purple), and T7-RNAP phage (green) on host cells harboring the PE PACE circuit before pegRNA optimization. Bars reflect the mean of n=3 independent replicates. Dots show individual replicate values. FIG. 2C shows a screen of pegRNAs for the v1 PE PACE circuit. Overnight propagation values of empty phage (red), NpuC-PE2-RT phage (purple), and T7-RNAP phage (green) are shown. Each point reflects the mean value of n=3 independent biological replicates for a different pegRNA. Individual replicates are shown in FIG. 9C.

FIG. 2D shows overnight propagation of empty phage (red), NpuC-PE1-RT phage (light purple), NpuC-PE2-RT phage (dark purple), and T7-RNAP phage (green) in the v1 pegRNA-optimized circuit. Bars reflect the mean of n=3 independent replicates. Dots show individual replicate values. FIG. 2E shows PANCE titers for the evolution of NpuC-PE1-RT phage. Grey shading indicates a passage of evolutionary drift, in which phage were supplied gIII in the absence of selection to allow free mutagenic replication. Titers of four replicate lagoons are shown. FIG. 2F shows a mutation table for RT clones that enriched during PANCE of NpuC-PE1-RT phage. Four clones from each lagoon (L1-L4, with clones ordered by lagoon) were sequenced. Light purple denotes a conserved mutation. Dark purple denotes a conserved mutation that was also present in the previously engineered RT in PE2¹. FIG. 2G shows a schematic of the PE PACE selection for evolution of the whole prime editor, including the Cas9 domain. The P1 plasmid (green) and P3 plasmid (orange) are identical to those used in FIG. 2A. FIG. 2H shows a PANCE experiment to compare the outcome of selection on v1 (requiring a 1-bp insertion) and v2 (requiring a 20-bp insertion) selection circuits. Whole-editor phage were divided into 16 separate lagoons, with 8 lagoons evolved on the v1 circuit (yellow) and 8 lagoons evolved on the v2 circuit (blue). After 31 passages, clones from each selection were Sanger sequenced, and the resulting mutations were compared to generate FIGS. 2D-2F. From top to bottom, the sequences are SEQ ID NOs: 134-137. FIG. 2I shows violin plots showing the number of mutations per clone for the M-MLV domain of whole-editor phage evolved with either the v1 (yellow) or v2 (blue) circuit. Data are shown as individual values, with one dot representing one sequenced phage. The mean value is shown as a dotted line. FIG. 2J shows predicted positions of mutated residues in M-MLV from v1 (yellow) or v2 (blue) PANCE. The structure is from the highly homologous XMRV (PDB: 4HKQ). FIG. 2K shows overnight propagation of pools of wild-type RT and evolved RT phage on their cognate or noncognate host-cell selection strains. Phage were from PANCE on the v1 circuit (yellow bars), from PANCE on the v2 circuit (blue bars), or wild-type-PE2 phage (grey bars). Propagation was then measured in the v1 circuit (left) or the v2 circuit (right). Bars reflect the mean of n=3 independent replicates. Dots show individual replicate values.

FIGS. 3A-3H: Phage-assisted evolution of compact RTs for prime editing. FIG. 3A shows a summary of evolution campaigns for NpuC-RT phage encoding Gs RT, Ec48 RT, or Tf1 RT. Shading indicates which selection circuit (v1 in yellow, v2 in blue, and v3 in purple) was used. Whether a given evolution was PANCE or PACE is specified: the number in parentheses after a PANCE or PACE label specifies how many passages of PANCE were performed (p) or how many hours of PACE (h) were performed. Arrowheads indicate that an evolution was stopped and increased in stringency without mammalian characterization; mutants characterized in mammalian cells are denoted with a dot and labeled. Finally, evolutions that used extra manipulations to increase stringency are labeled in pink, reflecting either a change in the PBS or a change in the expression of the target T7 RNAP gene. FIG. 3B shows the position of residues in the Gs RT close to the DNA⋅RNA substrate that were mutated following evolution mapped onto the structure of the Gs RT (PDB: 6AR1). Residues mutated following PANCE in the v2 circuit are red, residues mutated following PANCE and PACE in the v1 circuit are blue, the DNA substrate is green, and the RNA substrate is yellow. FIG. 3C shows predicted positions of residues in the Ec48 RT close to the DNA⋅RNA substrate (E60, E279, and K318) that were mutated after PANCE in the v1 and v2 circuit. Residues are mapped onto the AlphaFold predicted structure of the Ec48 overlayed with the substrate of the XMRV RT (PDB: 4HKQ). Residue mutated following PANCE in the v1 circuit is blue, residues mutated following PANCE in the v2 circuit are red, the DNA substrate is green, and the RNA substrate is yellow. FIG. 3D shows predicted positions of several conserved residues in the Tf1 RT that were mutated after PANCE in the v1, v2, and v3 circuit. Residues are mapped onto the AlphaFold predicted structure of the Tf1 RT overlayed with the substrate of the Ty3 RT (PDB: 40L8). Residues S492, K413, 1128, and K118 are all predicted to be close to the substrate while residues P70, G72, M102, and K106 decorate the surface of the enzyme that may be important for its interaction with the RTT of the pegRNA. Residues mutated following PANCE in the v1 circuit are blue, residues mutated following PANCE in the v2 circuit are red, and residues mutated following PANCE in the v3 circuit are in orange. The DNA substrate is green, and the RNA substrate is yellow. FIG. 3E shows prime editing using prime editors containing wild-type (grey) Gs, Ec48, and Tf1 RTs, evolved Gs-RT (evoGs, green), evolved Ec48 RT (evoEc48, blue), and evolved Tf1 RT (evoTf1, yellow) in HEK293T cells. Bars reflect the mean of n=3 independent replicates. Dots show individual replicate values. Throughout all figures, prime editing efficiencies shown reflect the frequency of the intended prime editing outcome with no indels at the target site. FIG. 3F shows a comparison of prime editors in the optimized PEmax architecture containing either engineered pentamutant Marathon RT (Marathon penta, red), evoEc48 (blue), or evoTf1 (yellow) with PEmax (gray) in HEK293T cells. Bars reflect the mean of n=3 independent replicates. Dots show individual replicate values. FIG. 3G shows prime editing in primary human T-cells at commonly edited test loci. Bars reflect the mean of n=4 independent replicates. Dots show individual replicate values. Indel-free editing is shown in blue or pink, and indels are shown in grey. FIG. 3H shows correction of the HEXA 1278insTATC mutation that causes Tay-Sachs disease in a HEK293T cell line model previously engineered to harbor the mutation (left) and in patient-derived fibroblasts (right). Bars reflect the mean of n=3 independent replicates for the HEK293T cell in model. Bars reflect n=2 independent replicates for the patient-derived fibroblasts. Dots show individual replicate values.

FIGS. 4A-4J: Evolved prime editor preferences and summary of RT evolution campaigns described herein. FIG. 4A shows a summary of evolution and engineering campaigns used to generate PE6c and PE6d. FIG. 4B shows conserved mutations from M-MLV RT evolution. The structure of XMRV RT (PDIB 4HKQ), which is highly homologous to M-MLV, shows PACE-evolved residues (blue) lie close to the enzyme active site (dark grey) and DNA/RNA duplex substrate (pink/purple). An incoming dNTP is shown in yellow. Below, pink lines indicate locations in the M-MLV RT at which PACE-evolved mutations truncated the protein. FIG. 4C shows fold-change in editing efficiency relative to PEmax for PEmaxΔRNaseH, PE6c, and PE6d in HEK293T cells. Individual replicates are plotted, with n=3 biological replicates per edit. FIG. 4D shows editing efficiencies of PEmaxΔRNaseH and PE6d at the HEK3+1 loxP insertion edit (pink) and the HEK3+1 FLAG insertion edit (orange) in HEK293T cells. Bars reflect the mean of n=3 independent replicates. Dots show individual replicate values. The NUPACK-predicted structures of the RTT and PBS extensions for each edit are shown. FIG. 4E shows results of a TdT assay on the HEK3+1 loxP insertion edit in HEK293T cells. The y-axis indicates the percentage of total RT products of a given length, and the x-axis represents the length of the product in base pairs. PEmaxΔRNaseH is shown in grey, and PE6d is shown in blue. The lines are mean values from n=3 biological replicates. The pink box indicates DNA bases templated by the structured portions of the pegRNA. FIG. 4F shows editing efficiencies of PEmaxΔRNaseH (grey) and PE6d (blue) at an example engineered hairpin edit and its corresponding unpinned control in HEK293T cells. The sequence of the RTT is shown (from top to bottom, SEQ ID NOs: 138 and 139), with point mutations in the unpinned control shown in red. Bars reflect the mean of n=3 independent replicates. Dots show individual replicate values. The NUPACK-predicted structures of the RTT and PBS extensions for each edit is shown. FIG. 4G shows the relationship between pegRNA RTT/PBS secondary structure and PE6d improvements. The y-axis reflects the fold-improvement of PE6d over PEmaxΔRNaseH. The x-axis is the absolute value of the free energy of pegRNA folding as measured by NUPACK. Each dot represents one edit in HEK293T cells that was calculated from the mean values from n=3 biological replicates. See FIG. 11D for individual editing values and edit identities. FIG. 4H shows a comparison of evolved and engineered RTs to PEmaxΔRNaseH at typical twinPE edits in HEK293T cells. Bars reflect the mean indel-free editing efficiency of n=3 independent replicates. Dots show individual replicate values. Solid bars indicate editing efficiency. Striped bars indicate indels. FIG. 4I shows twinPE-mediated insertion of the 38-bp attB sequence into the Rosa26 locus in N2a cells. Indel-free editing is shown in yellow, and indels are shown in grey. Bars reflect the mean of n=3 independent replicates. Dots show individual replicate values. FIG. 4J shows PE-mediated insertion of a 42-bp sequence containing loxP into the Dnmt1 locus in N2a cells. Indel-free editing is shown in yellow, and indels are shown in grey. Bars reflect the mean of n=3 independent replicates. Dots show individual replicate values.

FIGS. 5A-5H: Characterization of PE6 variants compared with PEmax. FIG. 5A shows prime editing efficiencies of PE6c, PE6d, and PEmax at challenging twinPE edits in HEK293T cells. Bars reflect the mean indel-free editing efficiency of n=3 independent replicates. Dots show individual replicate values. FIG. 5B shows edit to indel ratios of PE6c, PE6d, and PEmax at sites shown in 5A in HEK293T cells. Bars reflect the mean of n=3 independent replicates. Dots show individual replicate values. FIG. 5C shows twin prime editing in primary human T-cells at the CCR5 safe harbor locus. Indel-free editing is shown in red, and indels are shown in grey. Bars reflect the mean of n=4 independent replicates. Dots show individual replicate values. FIG. 5D shows edit to indel ratios of PE6b and PEmaxΔRNaseH normalized to that of PEmax in HEK293T cells. Individual replicates are plotted, with n=3 biological replicates per edit. Lines reflect the mean across all edits and replicates. Individual editing efficiencies and indel levels are shown in FIGS. 12D-12E. FIG. 5E shows edit to indel ratios of prime editors at endogenous HEK293T sites. The editor with the highest edit:indel ratio was picked and plotted side-by-side with PEmax for each specific edit. Bars reflect the mean of n=3 independent replicates. Dots show individual replicate values. Individual editing efficiencies and indel levels are shown in FIGS. 12D-12E. FIG. 5F shows prime editing efficiencies of PE6b and PE6c normalized to the editing efficiency of PEmax at 77 edits that install a pathogenic allele into endogenous sites in HEK293T cells. No nicking gRNA was used and MLH1dn plasmid was simultaneously transfected with prime editor plasmid for all conditions. All values from n=3 replicates are shown. Lines reflect the mean across all edits and replicates. Prime editing efficiencies for edits where PE6b or PE6c outperformed PEmax by more than 1.5-fold are shown on the right. Bars reflect the mean of n=3 independent replicates. Dots show individual replicate values. Prime editing efficiencies used are the frequency of the intended prime editing outcome with no indels or other changes at the target site. FIG. 5G shows correction of pathogenic mutations implicated in Crigler-Najjar Syndrome, Bloom Syndrome, and Pompe disease in HEK293T cell models using PEmax, PE6b, and PE6c. Bars reflect the mean of n=3 independent replicates. Dots show individual replicate values. FIG. 5H shows correction of mutations implicated in Crigler-Najjar Syndrome (UGT1A1) and Bloom Syndrome (RECQL3) in patient-derived fibroblast using PE6c and PEmax. Bars reflect the mean of n=3 independent replicates for treated samples and n=1-3 replicates of an untreated control for editing (red) and indels (gray). Dots show individual replicate values.

FIGS. 6A-6G: Evolution and engineering of improved Cas9 domains for prime editing, and summary of PE6 use. FIG. 6A shows a summary of evolution campaigns for whole PE2 phage. Shading indicates which circuit (v1 in yellow, v2 in blue, and v3 in purple) an evolution was performed in. Green shading indicates reversion analysis. Whether a given evolution was PANCE or PACE is specified: the number in parentheses after a PANCE or PACE label specifies how many passages of PANCE (p) were performed or how many hours of PACE (h) were performed. Arrowheads indicate that an evolution was stopped and increased in stringency without mammalian characterization; mutants characterized in mammalian cells are denoted with a dot and labeled. Finally, evolutions that utilized extra manipulations to increase stringency are labeled in pink, reflecting either a change in the PBS or a change in the expression of the target T7 RNAP gene. FIG. 6B shows an evaluation of PACE-evolved clones in HEK293T cells. Bars reflect the mean of n=3 independent replicates. Dots show individual replicate values. EvoCas9-1 through evoCas9-4 were isolated from low-stringency evolution. EvoCas9-5 and evoCas9-6 were isolated from high-stringency evolution. FIG. 6C shows an assessment of individual Cas9 mutations on prime editing efficiency at two test sites. The y-axis shows editing efficiency at the Pcsk9+3 C to G and +6 G to C edit in N2a cells. The x-axis shows editing efficiency for the RNF2+5 G to T edit in HEK293T cells. Mutants incorporated into final Cas9 variants are shown in green. Mutants previously shown to, or structurally predicted to, decrease Cas9 binding are shown in maroon. PEmaxΔRNaseH is shown in orange FIG. 6D shows a comparison of combined Cas9 mutants to PEmaxΔRNaseH in HEK293T cells and N2a cells. Editing efficiencies of variants are normalized to the editing efficiency generated by PEmaxΔRNaseH. Individual replicates are plotted, with n=3 biological replicates per edit. FIG. 6E shows a comparison of PEmax, PE6a, and PE6a/e at two sites in HEK293T cells. Bars reflect the mean of n=3 independent replicates. Dots show individual replicate values. FIG. 6F shows a comparison of PEmaxΔRNaseH, PE6c, and PE6g in HEK293T cells. Bars reflect the mean of n=3 independent replicates. Dots show individual replicate values. FIG. 6G shows a decision tree for selecting a PE6 variant.

FIGS. 7A-7C: PE6 variants enable new classes of in vivo prime editing. FIG. 7A is a schematic showing a dual-AAV delivery system for twinPE (v3em twinPE-AAV). In the N-terminal AAV, production of the N-terminal portion of Cas9 (yellow) fused to an N-terminal Npu split intein (orange) is regulated by the Cbh promoter (green) and the SV40 late polA signal (tan). In the C-terminal AAV, the C-terminal Npu split intein (dark green) is fused to the remainder of the prime editor (Cas9, yellow and RT, purple). The SV40 late polyA signal (tan), two epegRNAs (light and dark blue, AAV ITRs (black) are also shown. FIG. 7B shows an injection route and twinPE editing efficiency of PEmaxΔRNaseH and PE6d viruses in the for the twinPE-mediated insertion of a 38-bp attB sequence at murine Rosa26 in the mouse cortex. N- and C-terminal twinPE viruses are administered via ICV injection (4×10¹⁰ vg total) along with a GFP-KASH virus. Editing efficiencies (light and dark blue) and indel frequencies (black and grey) are shown to the right. Bars reflect the mean of n=3-4 mice. Dots show individual mice. FIG. 7C shows an injection route and PE editing efficiency of PEmaxΔRNaseH and PE6d viruses for the installation of a 42-bp insertion containing loxP at the Dnmt1 locus in the mouse cortex. (Left) The C-terminal virus is modified to include one epegRNA and one nicking sgRNA to encode a PE edit as opposed to a twinPE edit. (Right) Editing efficiencies (light/dark pink) and indel rates (black/grey). Bars reflect the mean of n=3 mice. Dots show individual mice.

FIGS. 8A-8J: Characterization and engineering of reverse transcriptase enzymes for prime editing, related to FIGS. 1A-1J. FIG. 8A show that native small RT enzymes demonstrate poor activity in the prime editing system (HEK293T cells, HEK3+5 G to T edit). RT enzymes engineered in FIGS. 1A-1J are highlighted in green, and the WT M-MLV RT used in the PE1 system is highlighted in black. All other enzymes are in red. Dots reflect the mean of n=3 independent replicates. FIG. 8B shows an overview of twinPE. The prime editor protein (grey and blue) uses two pegRNAs (dark blue and teal) to target opposite strands of DNA. The prime editor generates two 3′ flaps (red) that are complementary to each other. After these newly synthesized 3′ flaps anneal and the original DNA sequence in the 5′ flaps is degraded, the edited sequence in the flaps is permanently installed at the target DNA site. FIG. 8C shows incorporation of each of the five mutations analogous to those in PE2 improves the activity of four retroviral RT enzymes in HEK293T cells. PERV=porcine endogenous retrovirus RT, AVIRE=avian reticuloendotheliosis virus RT, KORV=koala retrovirus RT and WMSV=woolly monkey sarcoma virus RT. Combining all five mutations together (Penta) further improves the activity of each enzyme. All values from n=3 independent replicates are shown. Horizontal bars show the mean value. FIG. 8D shows structure-guided rational engineering of the Tf1 RT identifies five mutations that improve prime editing in HEK293T cells. The solved structure of the Tf1 RT homolog, Ty3 RT, was used to predict mutations that could increase contacts of the RT with its DNA-RNA substrate (PDB: 40L8). All values from n=3 independent replicates are shown. Horizontal bars show the mean value across all sites and replicates. FIG. 8E shows combining all mutations identified from structure-guided rational engineering improves the activity of the Tf1 RT prime editor in HEK293T cells. The final rationally designed Tf1 variant (rdTf1) is a combination of five mutations: K118R, S188K, I260L, R288Q, and S297Q. All values from n=3 independent replicates are shown. Horizontal bars show the mean value. FIG. 8F shows an AlphaFold-predicted structure of the Ec48 RT enzyme. FIG. 8G shows that aligning the AlphaFold-predicted structure of the Ec48 RT (blue) with the RT from xenotropic murine leukemia virus-related virus (XMRV, PDB 4HKQ, yellow), a close relative of the M-MLV RT, suggests that the residue analogous to the D200 residue in M-MLV RT is the T189 residue in Ec48 RT. FIG. 8H shows structure-guided rational engineering of the Ec48 RT identifies six mutations that improve prime editing. An AlphaFold-generated predicted structure of the Ec48 RT was overlayed with the structure of the RT from the xenotropic murine leukemia virus-related virus (XMRV) (PDB: 4HKQ) to perform structure-guided mutagenesis. All values from n=3 independent replicates are shown. Horizontal bars show the mean value. FIG. 8I shows the positions of residues (red) proximal to the substrate that were mutated to improve the activity of the Ec48 RT prime editor. Residues are mapped onto the predicted AlphaFold structure of the Ec48 RT aligned with the solved substrate of the XMRV RT (PDB: 4HKQ). L182 and T385 are proximal to the DNA substrate (green), R315 and K307 are proximal to the RNA substrate (yellow) and R378 is proximal to both the DNA and RNA rate. FIG. 8J shows that combining the top three mutations identified from structure-guided engineering improves the activity of the Ec48 RT prime editor in HEK293T cells. The final rationally designed Ec48 RT variant (rdEc48) contains three mutations: L182N, T189N, and R315K. All values from n=3 independent replicates are shown. Horizontal bars show the mean value.

FIGS. 9A-9F: Design and validation of a PE PACE circuit, related to FIGS. 2A-2K. FIG. 9A shows a summary of phage-assisted continuous evolution (PACE). Host E. coli (grey) harboring relevant selection circuit plasmids (green, pink, and orange) and the mutagenesis plasmid (MP, black) continuously flow into a fixed-volume lagoon (left). Addition of arabinose induces expression of mutagenic genes on the MP. Selection phage (blue) harboring an NpuC-RT transgene (purple) infect the E. coli and are mutagenized. If a mutagenized RT is inactive (red, bottom/right), then prime editing does not trigger gIII expression and pIII production, and phage are not able to propagate. These phage encoding inactive RTs are washed out of the lagoon by continuous flow. If a mutagenized RT is active (green, center), then prime editing leads to pIII production, and phage encoding that RT can propagate faster than the rate at which they are diluted out of the lagoon. FIG. 9B shows a summary of phage-assisted non-continuous evolution (PANCE). The same principles shown in FIG. 9A are used in PANCE, except periodic discrete dilution steps instead of continuous flow is used to dilute selection cultures. Mid-log-phase cultures of selection E. coli are infected with phage, and arabinose is added to induce mutagenesis (left). After an overnight incubation, cultures are centrifuged to pellet bacteria and allow isolation of propagating phage from the supernatant (middle). A small volume of supernatant (typically a 1:50 dilution factor) is used to infect a fresh lagoon of mid-log selection strains (right). This process is iterated until phage titers stabilize (i.e., when overnight phage propagation is equal to or greater than the dilution factor). FIG. 9C shows the effect of pegRNA optimization on PE2 phage propagation. Overnight propagation of empty phage (native control, red), PE2 phage (purple), and T7 RNAP phage (positive control, green) in strains harboring pegRNAs of different PBS and RTT lengths. Bars reflect the mean of n=3 independent replicates. Dots show individual replicate values. This data was used to generate FIG. 2C. FIG. 9D shows a luciferase assay to screen pegRNAs for the v2 PE PACE circuit. Selection strains encoding luxAB transcriptionally coupled to gIII were infected with either empty phage (red) or PE2 phage (purple). 4 h after infection, OD₆₀₀-normalized luminescence was measured as a proxy for circuit activation. Bars reflect the mean of n=3 independent replicates. Dots show individual replicate values. Strains in which PE2 phage outperformed empty phage were used for v2 evolutions. FIG. 9E shows overnight propagation of pools of wild-type RT and evolved RT phage on their cognate or noncognate host-cell selection strains. Additional evolved pools of phage are shown here beyond those provided in FIG. 2K. Phage were from PANCE on the v1 circuit (yellow bars), from PANCE on the v2 circuit (blue bars), or wild-type-PE2 phage (grey bars). Propagation was then measured in the v1 circuit (left) or the v2 circuit (right). Bars reflect the mean of n=3 independent replicates. Dots show individual replicate values. FIG. 9F shows the design of v3 circuit and improvements compared to v1 and v2 designs. A long insertion edit (20-bp insertion edit with a 60-bp RTT) was used to select for high-processivity, high-activity prime editors. Unlike v1 and v2 circuits, the v3 pegRNA (grey) targets the noncoding strand of T7 RNAP; this shortens the time between prime editing and wild type T7 RNAP production. In addition to the 20-bp insertion (green) needed to restore the frame of T7 RNAP, the v3 pegRNA also encodes silent PAM edits (maroon) and a seed edit (blue) that prevents subsequent binding and nicking of the edited sequence. From top to bottom, the sequences are SEQ ID NOs: 140 and 141.

FIGS. 10A-10F: Evolution and characterization of compact RTs for prime editing, related to FIGS. 3A-3H. FIG. 10A shows overnight propagation of phage encoding dead M-MLV RT (red), Gs (blue), or PE2 (purple) RTs in the NpuC-RT phage architecture in the pegRNA-optimized v1 PE PACE circuit. Bars reflect the mean of n=3 independent replicates. Dots show individual replicate values. FIG. 10B shows phage titers during PANCE of NpuC-Gs-RT phage. Grey shading indicates a passage of evolutionary drift, in which phage were supplied gIII in the absence of selection to allow free mutagenic replication. Titers of four replicate lagoons are shown. FIG. 10C shows PACE of NpuC-Gs-RT phage. The left y-axis and pink and blue lines show the SP titer of three different replicate lagoons at various timepoints. The right y-axis and dotted grey line show the flow rate in volumes per hour. FIG. 10D shows indel frequencies for prime editors in the optimized PEmax architecture containing either engineered pentamutant Marathon RT (Marathon penta, red), evoEc48 (blue), or evoTf1 (yellow) with PEmax (gray) in HEK293T cells. Editing frequencies corresponding to this data is in FIG. 3F. Bars reflect the mean of three independent replicates. Dots show individual replicate values. FIG. 10E shows performance of PE6a and PE6b in the presence and absence of epegRNAs in HEK293T cells. All values from n=3 independent replicates are shown. Horizontal bars show the mean value. FIG. 10F shows a comparison of PE6a, PE6b, and PEmax at three longer, complex edits in HEK293T cells. Bars reflect the mean of n=3 independent replicates. Dots show individual replicate values.

FIGS. 11A-11G: Development and characterization of highly processive, dual AAV-compatible RTs. FIG. 11A shows editing efficiencies of prime editors containing single M-MLV mutants in HEK293T cells. Prime editing efficiencies used are the frequency of the intended prime editing outcome with no indels or other changes at the target site. Lines reflect the mean of n=2 independent replicates per edit. Dots show individual replicate values. FIG. 11B shows an overview of the terminal deoxynucleotidyl transferase (TdT) assay for sequencing newly reverse-transcribed DNA flaps that have not been incorporated into the genome. Shortly after treatment with a prime editor and pegRNA, cells are lysed, and DNA is purified. A terminal transferase enzyme (yellow) adds a polyG sequence to all DNA 3′ ends. PCR amplification for high-throughput DNA sequencing is performed using a locus-specific forward primer and a polyC reverse primer. FIG. 11C shows results of a TdT assay on the HEK3+1 FLAG insertion edit in HEK293T cells. The y-axis indicates the percentage of total RT products of a given length, and the x-axis represents the length of the product in base pairs. PEmaxΔRNaseH is shown in grey, and PE6d is shown in blue. The lines are mean values from n=3 biological replicates. FIG. 11D shows editing efficiencies of PE6b-d, PEmax, and PEmaxΔRNaseH for edits engineered to contain varying levels of secondary structure. “UC” indicates an unpinned control for a corresponding hairpin edit. These values were used to generate the free energy vs fold improvement plot in FIG. 4G. All edits are in HEK293T cells. Individual replicates are shown, with n=3 replicates per condition. FIG. 11E shows editing efficiencies (left) and indel rates (right) of PE6d and PEmaxΔRNaseH for a series of prime edits that use short unstructured pegRNAs in HEK293T cells. Bars reflect the mean of n=3 independent replicates. Dots show individual replicate values. PEmaxΔRNaseH is shown on the right for each edit site, and PE6d is shown on the left for each edit site. FIG. 11F shows results of a TdT assay on the RNF2+5 G to T edit in HEK293T cells. Note that the x-axis differs from other TdT plots shown herein: instead of RTT-templated bases correctly installed, it quantifies the number of sgRNA scaffold-templated bases aberrantly installed (for example, x=1 indicates the addition of one extra scaffold-templated base). The y-axis indicates the percentage of edit-containing flaps that have a given number of scaffold-templated bases. For each prime editor, the line reflects the mean of n=3 independent replicates. Pie charts indicate the percentages of edit-containing flaps that either have ≤2 bp (solid color) or >2 bp (striped) of scaffold-templated bases. Data shown are the mean of three independent biological replicates. FIG. 11G shows unique molecular identifier (UMI) analysis of prime editing efficiencies for twinPE edits in N2a cells (left) and HEK293T cells (middle, right). UMI protocol was applied to remove PCR bias, and trends agree with the data shown in FIGS. 4A-4J. Bars reflect the mean of n=3 independent replicates. Dots show individual replicate values. For each instance of “Edit” and “Indels,” PEmaxΔRNaseH is shown on the left, PE6c is shown in the middle, and PE6d is shown on the right.

FIGS. 12A-12J: Comparison of PE6 variants with PEmax, related to FIGS. 5A-5H. FIG. 12A shows prime editing efficiencies of the best performing PE6 variant (either PE6c or PE6d) normalized to the editing efficiency of PEmax at sites tested in FIG. 5A. All values from n=3 independent replicates are shown. Editing was performed in HEK293T cells. The horizontal bar shows the mean value. FIG. 12B shows indel frequencies of PEmax, PE6c, and PE6d at edits tested in FIG. 5A. This data was used for FIG. 5B. Bars reflect the mean of three independent replicates. Editing was performed in HEK293T cells. Dots show individual replicate values. FIG. 12C shows screening PE6 variants for insertion of attB into the CCR5 locus in primary human T cells. Bars reflect the mean of n=4 independent replicates for editing (red) and indels (grey). Dots show individual replicate values. FIG. 12D shows absolute prime editing efficiencies of PE6 variants, PEmaxΔRNaseH, and PEmax in HEK293T cells used to plot data for FIGS. 5D-5E. Prime editing efficiencies used are the frequency of the intended prime editing outcome with no indels or other changes at the target site. Bars reflect the mean of three independent replicates. Dots show individual replicate values. FIG. 12E shows indel frequencies of PE6 variants, PEmaxΔRNaseH, and PEmax in HEK293T cells used to plot data for FIGS. 5D-5E. Bars reflect the mean of three independent replicates. Dots show individual replicate values. FIG. 12F shows a percentage of sequencing reads containing a pegRNA scaffold insertion after prime editing using PE6 variants, PEmaxΔRNaseH, and PEmax in HEK293T cells. These reads contribute to the total indel frequency. Bars reflect the mean of n=3 independent replicates. Dots show individual replicate values. FIG. 12G shows prime editing efficiencies for edits where PE6b or PE6c outperformed PEmax using a nicking gRNA. Bars reflect the mean of n=3 independent replicates. Dots show individual replicate values. Prime editing efficiencies used are the frequency of the intended prime editing outcome with no indels or other changes at the target site in HEK293T cells. FIG. 12H shows indel frequencies of PE6 variant and PEmax at sites shown in FIG. 5F in HEK293T cells. Bars reflect the mean of n=3 independent replicates. Dots show individual replicate values. FIG. 12I shows a correction of mutation implicated in Pompe disease in patient-derived fibroblast using PE6c and PEmax. Bars reflect the mean of n=3 independent replicates for editing (red) and indels (grey). Dots show individual replicate values. FIG. 12J shows a distribution of editing outcomes after correction of the pathogenic mutation implicated in Pompe disease in patient-derived fibroblasts using PE6c. The patient was heterozygous. Indel genotypes are shown. From top to bottom, the sequences are SEQ ID NOs: 142, 143, 144, 144, 142, 144, 31, 143, and 144.

FIGS. 13A-13F: Evolution and engineering of Cas9 mutants for PE, related to FIGS. 6A-6G. FIG. 13A shows a representative PACE campaign for the v1 circuit. Different colored lines represent different replicate lagoons. PACE experiments with less than four lagoons shown experienced cheating (activity-independent phage propagation likely from rare gene III recombination onto the SP) or washout (complete loss of viable phage) for one or more lagoons. Top graphs represent the phage titer over a PACE experiment. Bottom graphs show the flow rate at the corresponding time. FIG. 13B shows a reversion analysis of EvoCas9-4 in HEK293T cells. Editing efficiency was normalized to the values obtained using PE2. Data are shown as individual data points for n=3 biological replicates and as the grand mean across the four sites tested. FIG. 13C shows a structural analysis of mutations that harm mammalian prime editing activity. (Left) Structure (PDB: 4UN3) of wild-type Sp Cas9 (grey) bound to its guide RNA (purple) and DNA substrate (yellow/orange). Residue K1151 is shown in dark pink. (Right) Structure (PDB: 4008) of wild-type Sp Cas9 (grey) bound to its guide RNA (purple) and DNA substrate (orange). Wild-type residues K1003, K1014, and A1034 are shown in dark pink. FIG. 13D shows a circuit for examining editing-independent effects of the prime editor on the PE PACE circuit. E. coli harbor a corrected T7 RNAP, luxAB gene under the T7 promoter, and the pegRNA used during selection. Prime editor variants are introduced on a plasmid under the control of an arabinose-inducible promoter. After induction, OD-normalized luminescence for n=3 biological replicates were used to measure circuit turn on. Bars reflect the mean of n=3 independent replicates. Dots show individual replicate values. FIG. 13E shows prime editing efficiencies N2a cells (left, Ctnnb1 through Pcks9) and HEK293T cells (right, CXCR4 through RNF2) used to generate the fold changes reported in FIG. 6D. Individual replicates are plotted, with n=3 biological replicates per edit. FIG. 13F show the structure (PDB: 4UN3) of Cas9 (grey) bound to its sgRNA (purple). Residue H721, which is mutated to Tyr in evolutions, is shown in green sticks. Dotted lines denote predicted polar contacts between H721 and other atoms.

FIGS. 14A-14E: In vivo prime editing with PE6c and PE6d delivered via dual AAV, related to FIGS. 7A-7C. FIG. 14A shows an analysis of truncated PE6c variants. Editing (yellow) and indels (grey) are shown for the installation of an attB sequence at the murine Rosa26 locus in N2a cells. Bars reflect the mean of n=3 independent replicates. Dots show individual replicate values. The number below each variant indicates the number of DNA bases that have been deleted from the C-terminal end of the Tf1 gene. FIG. 14B shows representative flow plots for the isolation of unsorted and sorted nuclei from mouse cortices. Left: scatter plot of all events, gate A set to collect nuclei. Middle: selection of single-nuclei droplets in Gate B, Right: FITC signal was used to collect unsorted cells (Gate C) and transduced, GFP-positive cells (Gate D). FIG. 14C shows twinPE editing efficiency of PEmaxΔRNaseH and PE6c viruses in the mouse cortex. N- and C-terminal twinPE viruses are administered via ICV injection (4×10¹⁰ vg total) along with a GFP-KASH virus. Editing efficiencies (light and dark blue) and indel (black/grey) rates are shown to the right. Bars reflect the mean of n=3-4 mice. Dots show individual mice. FIG. 14D shows injection route and PE editing (Dnmt1 loxP insertion) efficiency of PEmaxΔRNaseH and PE6d viruses at a low viral dose (2×10¹⁰ vg total) in the mouse cortex. (Left) The C-terminal virus is modified to include one epegRNA and one nicking sgRNA to encode a PE edit as opposed to a twinPE edit. (Right) Editing efficiencies (light/dark pink) and indel rates (black/grey). Bars reflect the mean of n=3 mice. Dots show individual mice. FIG. 14E shows off-target editing from AAV-treated and untreated mice. Bars reflect the mean of n=3 mice. Dots show individual mice. PE6d bulk (light pink) and transduced (dark pink) values were either less than 0.1% on average or were not statistically significant from untreated controls (light grey). For both ns notes, p=0.08. Analyses were performed with an unpaired t test with Welch correction. The y-axis indicates off-target editing and indels summed (see methods for calculation). OT6 failed to amplify. All treated samples are from the high AAV dose condition.

FIGS. 15A-15B: Mutation tables from v1 PE PA(N)CE Gs evolution, related to FIGS. 3A-3H. Mutations in clones emerging from evolution. Lagoon 2 cheated during PACE and was not sequenced. Silent mutations omitted for clarity.

FIG. 16: Mutation table from v1 PE PANCE Tf1 evolution, related to FIGS. 3A-3H. Mutations in clones emerging from evolution. Silent mutations omitted for clarity.

FIG. 17: Mutation table from v1 PE PANCE Ec48 evolution, related to FIGS. 3A-3H. Mutations in clones emerging from evolution. Silent mutations omitted for clarity.

FIG. 18: Mutation table from v1 PE PANCE Vc95 evolution, related to FIGS. 3A-3H. Mutations in clones emerging from evolution. Silent mutations omitted for clarity.

FIGS. 19A-19J: Mutation tables from v1 whole editor evolution, related to FIGS. 4A-4G. Mutations in clones emerging from evolution. Blue shading indicates an amino acid change, and orange shading indicates a truncating mutation, either a stop codon (*) or a frameshift (FS). Silent mutations omitted for clarity.

FIG. 20: Mutation table from v1 and v2 comparative PE PANCE whole editor evolution, related to FIGS. 4A-4G. Mutations in clones emerging from evolution. Mutations from v1 are shaded in yellow, and mutations from v2 evolution are shaded in blue. Silent mutations omitted for clarity. Frameshift mutations are not shown.

FIG. 21: Mutation table from v2 PE PANCE Gs evolution, related to FIGS. 4A-4G. Mutations in clones emerging from evolution. Silent mutations omitted for clarity.

FIG. 22: Mutation table from v2 PE PANCE Ec48 evolution, related to FIGS. 4A-4G. Mutations in clones emerging from evolution. Silent mutations omitted for clarity.

FIG. 23: Mutation table from v2 high stringency PE PANCE Ec48 evolution, related to FIGS. 4A-4G. Mutations in clones emerging from evolution. Silent mutations omitted for clarity.

FIG. 24: Mutation table from v2 PE PANCE Tf1 evolution, related to FIGS. 4A-4G. Mutations in clones emerging from evolution. Silent mutations omitted for clarity.

FIG. 25: Mutation table from v3 PE PANCE Tf1 evolution, related to FIGS. 4A-4G. Mutations in clones emerging from evolution. Silent mutations omitted for clarity.

FIG. 26: Mutation table from v1 PE PACE PE2 RT evolution, related to FIGS. 4A-4G. Mutations in clones emerging from evolution. Silent mutations omitted for clarity.

FIG. 27: Mutation table from v2 PE PANCE PE2 RT evolution, related to FIGS. 4A-4G. Mutations in clones emerging from evolution. Silent mutations omitted for clarity.

FIGS. 28A-28C: Mutation tables from v3 PE PANCE PE2 RT evolution, related to FIGS. 4A-4G. Mutations in clones emerging from evolution. Silent mutations omitted for clarity.

FIGS. 29A-29F: Mutation tables from v1-v3 whole editor evolutions, related to FIGS. 6A-6F. Mutations in clones emerging from evolution. Silent mutations omitted for clarity.

FIG. 30 shows neonatal cerebroventricular (P0 ICV) injections of dual AAV delivering PEmaxΔRNaseH or PE6 variants to the CNS of 12 mice (n=4 for each of three groups).

FIG. 31 shows in vivo liver editing after ICV injection. PE6 editors substantially enhance liver editing (30% editing in the liver after ICV injection).

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### Adeno-Associated Virus (AAV)

An “adeno-associated virus” or “AAV” is a virus that infects humans and some other primate species. The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2, and VP3, which interact together to form the viral capsid. VP1, VP2, and VP3 are translated from one mRNA transcript, which can be spliced in two different manners: either a longer or shorter intron can be excised resulting in the formation of two isoforms of mRNAs: a ~2.3 kb- and a ~2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

Recombinant AAV (rAAV) particles may comprise a nucleic acid vector (e.g., a recombinant genome), which may comprise at a minimum: (a) one or more heterologous nucleic acid regions comprising a sequence encoding a protein or polypeptide of interest (e.g., a split prime editor) or an RNA of interest (e.g., a gRNA), or one or more nucleic acid regions comprising a sequence encoding a Rep protein; and (b) one or more regions comprising inverted terminal repeat (ITR) sequences (e.g., wild-type ITR sequences or engineered ITR sequences) flanking the one or more nucleic acid regions (e.g., heterologous nucleic acid regions). In some embodiments, the nucleic acid vector is between 4 kb and 5 kb in size (e.g., 4.2 to 4.7 kb in size). In some embodiments, the nucleic acid vector further comprises a region encoding a Rep protein. In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector.

In some embodiments, an AAV is used to deliver any of the reverse transcriptase variants, Cas9 variants, fusion proteins, prime editors, and/or polynucleotides or vectors encoding the same.

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain,” as used herein, is a protein fragment comprising an active or fully or partly inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids.

CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc), and a Cas9 domain. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The strand in the target DNA not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the contents of which are incorporated herein by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of a Cas9 protein are provided. For example, in some embodiments, a protein comprises one of two Cas9 domains: (1) the gRNA binding domain of Cas9; or (2) the DNA cleavage domain of Cas9. In some embodiments, proteins comprising Cas9, or fragments thereof, are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 6). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 6). In some embodiments, the Cas9 variant comprises a fragment of SEQ ID NO: 6 Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 6). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or at least 99.5% of the amino acid length of a corresponding wild type Cas9 (e.g., SpCas9 of SEQ ID NO: 6).

In some embodiments, a Cas9 protein comprises any of the amino acid substitutions described herein. In certain embodiments, a Cas9 protein comprises the amino acid substitutions K775R and K918A relative to wild type Streptococcus pyogenes Cas9 or relative to Streptococcus pyogenes Cas9 nickase (SEQ ID NO: 2). In certain embodiments, a Cas9 protein comprises the amino acid substitutions H99R, E471K, I632V, D645N, R654C, and H721Y relative to wild type Streptococcus pyogenes Cas9 or relative to Streptococcus pyogenes Cas9 nickase (SEQ ID NO: 2). In certain embodiments, a Cas9 protein comprises the amino acid substitutions H99R, E471K, I632V, D645N, H721Y, and K918A relative to wild type Streptococcus pyogenes Cas9 or relative to Streptococcus pyogenes Cas9 nickase (SEQ ID NO: 2).

### CRISPR

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (rnc), and a Cas9 protein. The tracrRNA serves as a guide for ribonuclease 3-aided processing of pre-crRNA. Subsequently, Cas9/crRNA/tracrRNA endonucleolytically cleaves a linear or circular dsDNA target complementary to the RNA. Specifically, the DNA strand in the target that is not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species—the guide RNA. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

In general, a “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr mate sequence (encompassing a “direct repeat” and a tracrRNA-processed partial direct repeat in the context of an endogenous CRISPR system), a guide sequence (also referred to as a “spacer”), or other sequences and transcripts from a CRISPR locus. The tracrRNA of the system is complementary (fully or partially) to the tracr mate sequence present on the guide RNA.

### Edit Strand and Non-Edit Strand

The terms “edit strand” and “non-edit strand” are terms that may be used when describing the mechanism of a prime editing system on a double-stranded DNA substrate. The “edit strand” refers to the strand of DNA that is nicked by the prime editor complex to form a 3′ end, which is then extended as a newly synthesized single stranded DNA (also referred to herein as the newly synthesized 3′ DNA flap), which comprises a desired edit and ultimately displaces and replaces the single strand region of DNA just downstream of the nick, thereby installing the 3′ DNA flap containing the desired edit downstream of the nick on the “edit strand.” In some embodiments, the newly synthesized 3′ DNA flap comprising the nucleotide edit is paired in a heteroduplex with the non-edit strand that does not comprise the nucleotide edit, thereby creating a mismatch. In some embodiments, the mismatch is recognized by DNA repair machinery, and/or replication machinery, e.g., an endogenous DNA repair machinery. In some embodiments, through DNA repair, the intended nucleotide edit is incorporated into both strands of the target double-stranded DNA substrate. The application may also refer to the “edit strand” as the “protospacer strand” or the “PAM strand” since these elements are present in that strand. The “edit strand” may also be called the “non-target strand” since the edit strand is not the strand that becomes annealed to the spacer of the pegRNA molecule, but rather is the complement of the strand that is annealed by the spacer of the pegRNA. The “non-edit” strand is not directly edited by the PE system. Rather, the desired edit created by the PE system in the 3′ DNA flap is incorporated into the “non-edited strand” through DNA replication and/or repair. In some embodiments, the “non-edit strand” is the strand that anneals to the spacer of the pegRNA, and thus is also called the “target strand.”

### Fusion Protein

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid-programmable DNA-binding domain (e.g., the gRNA binding domain of Cas9 that directs the binding of the protein to a target site) and a reverse transcriptase (i.e., a prime editor). In some embodiments, a fusion protein comprises any of the reverse transcriptase variants provided herein fused to at least one other domain (e.g., a Cas9 protein (such as a Cas9 variant provided herein), an NLS, or any other domain disclosed herein). In some embodiments, a fusion protein comprises any of the Cas9 variants provided herein fused to at least one other domain (e.g., a reverse transcriptase (such as a reverse transcriptase variant provided herein), an NLS, or any other domain disclosed herein). In certain embodiments, a fusion protein comprises any of the reverse transcriptase variants provided herein and any of the Cas9 variants provided herein. Any of the fusion proteins provided herein may be produced by any method known in the art. For example, the prime editor fusion proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), which is incorporated herein by reference.

### Genetic Elements of AAV Particle Vectors

Nucleic acids of the present disclosure (e.g., nucleic acids delivered by an AAV particle as described herein) may include one or more genetic elements. A “genetic element” refers to a particular nucleotide sequence that has a role in nucleic acid expression (e.g., promoter, enhancer, terminator) or encodes a discrete product of an engineered nucleic acid (e.g., a nucleotide sequence encoding a guide RNA and/or a protein).

A “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific, or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter is referred to as an “endogenous promoter.” In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR).

In some embodiments, promoters used in accordance with the present disclosure are “inducible promoters,” which are promoters that are characterized by regulating (e.g., initiating or activating) transcriptional activity when in the presence of, influenced by or contacted by an inducer signal. An inducer signal may be endogenous or a normally exogenous condition (e.g., light), compound (e.g., chemical or non-chemical compound), or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation of a repressor that is preventing the promoter from driving transcription. Conversely, deactivation of transcription may involve directly acting on a promoter to prevent transcription or indirectly acting on a promoter by activating a repressor that then acts on the promoter.

A “transcriptional terminator” is a nucleic acid sequence that causes transcription to stop. A transcriptional terminator may be unidirectional or bidirectional. It is comprised of a DNA sequence involved in specific termination of an RNA transcript by an RNA polymerase. A transcriptional terminator sequence prevents transcriptional activation of downstream nucleic acid sequences by upstream promoters. A transcriptional terminator may be necessary in vivo to achieve desirable expression levels or to avoid transcription of certain sequences. A transcriptional terminator is considered to be “operably linked to” a nucleotide sequence when it is able to terminate the transcription of the sequence it is linked to.

The most commonly used type of terminator is a forward terminator. When placed downstream of a nucleic acid sequence that is usually transcribed, a forward transcriptional terminator will cause transcription to abort. In some embodiments, bidirectional transcriptional terminators are provided, which usually cause transcription to terminate on both the forward and reverse strand. In some embodiments, reverse transcriptional terminators are provided, which usually terminate transcription on the reverse strand only.

In prokaryotic systems, terminators usually fall into two categories (1) rho-independent terminators and (2) rho-dependent terminators. Rho-independent terminators are generally composed of a palindromic sequence that forms a stem loop rich in G-C base pairs followed by several T bases. Without wishing to be bound by theory, the conventional model of transcriptional termination is that the stem loop causes RNA polymerase to pause, and transcription of the poly-A tail causes the RNA:DNA duplex to unwind and dissociate from RNA polymerase.

In eukaryotic systems, the terminator region may comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in some embodiments involving eukaryotes, a terminator may comprise a signal for the cleavage of the RNA. In some embodiments, the terminator signal promotes polyadenylation of the message. The terminator and/or polyadenylation site elements may serve to enhance output nucleic acid levels and/or to minimize read through between nucleic acids.

Terminators for use in accordance with the present disclosure include any terminator of transcription described herein or known to one of ordinary skill in the art. Examples of terminators include, without limitation, the termination sequences of genes such as, for example, the bovine growth hormone terminator, and viral termination sequences such as, for example, the SV40 terminator, spy, yejM, secG-leuU, thrLABC, rrnB T1, hisLGDCBHAFI, metZWV, rrnC, xapR, aspA, and arcA terminator. In some embodiments, the termination signal may be a sequence that cannot be transcribed or translated, such as those resulting from a sequence truncation.

### Linker

The term “linker,” as used herein, refers to a molecule linking two other molecules or moieties. The linker can be an amino acid sequence in the case of a peptide linker joining two domains of a fusion protein. For example, a napDNAbp (e.g., Cas9) can be fused to a reverse transcriptase by an amino acid linker sequence. The linker can also be a nucleotide sequence in the case of joining two nucleotide sequences together (e.g., in a gRNA). For example, in the instant case, the traditional guide RNA is linked via a spacer or linker nucleotide sequence to the RNA extension of a prime editing guide RNA which may comprise an RT template sequence and an RT primer binding site. In other embodiments, the linker is an organic molecule, group, polymer, or chemical moiety. In some embodiments, the linker is 5-200 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

napDNAbp

As used herein, the term “nucleic acid programmable DNA binding protein” or “napDNAbp,” of which Cas9 is an example, refers to a protein that uses RNA:DNA hybridization to target and bind to specific sequences in a DNA molecule. Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9 or equivalent) to localize and bind to a complementary sequence.

Without being bound by theory, the binding mechanism of a napDNAbp-guide RNA complex, in general, includes the step of forming an R-loop whereby the napDNAbp induces the unwinding of a double-strand DNA target, thereby separating the strands in the region bound by the napDNAbp. The guide RNA protospacer then hybridizes to the “target strand.” This displaces a “non-target strand” that is complementary to the target strand, which forms the single strand region of the R-loop. In some embodiments, the napDNAbp includes one or more nuclease activities, which then cut the DNA, leaving various types of lesions. For example, the napDNAbp may comprise a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”). Exemplary sequences for these and other napDNAbp are provided herein. In some embodiments, a napDNAbp has nickase activity in a RuvC domain and/or an HNH domain. In some embodiments, a napDNAbp has nickase activity in a RuvC domain or an HNH domain.

### Nickase

As used herein, a “nickase” refers to a napDNAbp (e.g., a Cas protein) which is capable of cleaving only one of the two complementary strands of a double-stranded target DNA sequence, thereby generating a nick in that strand. In some embodiments, the nickase cleaves a non-target strand of a double stranded target DNA sequence. In some embodiments, the nickase comprises an amino acid sequence with one or more mutations in a catalytic domain of a canonical napDNAbp (e.g., a Cas protein), wherein the one or more mutations reduces or abolishes nuclease activity of the catalytic domain. In some embodiments, the nickase is a Cas9 that comprises one or more mutations in a RuvC-like domain relative to a wild type Cas9 sequence or to an equivalent amino acid position in other Cas9 variants or Cas9 equivalents. In some embodiments, the nickase is a Cas9 that comprises one or more mutations in an HNH-like domain relative to a wild type Cas9 sequence or to an equivalent amino acid position in other Cas9 variants or Cas9 equivalents. In some embodiments, the nickase is a Cas9 that comprises an aspartate-to-alanine substitution (D10A) in the RuvC I catalytic domain of Cas9 relative to a canonical SpCas9 sequence or to an equivalent amino acid position in other Cas9 variants or Cas9 equivalents. In some embodiments, the nickase is a Cas9 that comprises an H840A, N854A, and/or N863A mutation relative to a canonical SpCas9 sequence, or to an equivalent amino acid position in other Cas9 variants or Cas9 equivalents. In some embodiments, the term “Cas9 nickase” refers to a Cas9 with one of the two nuclease domains inactivated. This enzyme is capable of cleaving only one strand of a target DNA. In some embodiments, the nickase is a Cas protein that is not a Cas9 nickase.

In some embodiments, the napDNAbp of the prime editing complex comprises an endonuclease having nucleic acid programmable DNA binding ability. In some embodiments, the napDNAbp comprises an active endonuclease capable of cleaving both strands of a double stranded target DNA. In some embodiments, the napDNAbp is a nuclease active endonuclease, e.g., a nuclease active Cas protein, that can cleave both strands of a double stranded target DNA by generating a nick on each strand. For example, a nuclease active Cas protein can generate a cleavage (a nick) on each strand of a double stranded target DNA. In some embodiments, the two nicks on both strands are staggered nicks, for example, generated by a napDNAbp comprising a Cas12a or Cas12b1. In some embodiments, the two nicks on both strands are at the same genomic position, for example, generated by a napDNAbp comprising a nuclease active Cas9. In some embodiments, the napDNAbp comprises an endonuclease that is a nickase. For example, in some embodiments, the napDNAbp comprises an endonuclease comprising one or more mutations that reduce nuclease activity of the endonuclease, rendering it a nickase. In some embodiments, the napDNAbp comprises an inactive endonuclease, for example, in some embodiments, the napDNAbp comprises an endonuclease comprising one or more mutations that abolish the nuclease activity. In various embodiments, the napDNAbp is a Cas9 protein or variant thereof. The napDNAbp can also be a nuclease active Cas9, a nuclease inactive Cas9 (dCas9), or a Cas9 nickase (nCas9). In a preferred embodiment, the napDNAbp is Cas9 nickase (nCas9) that nicks only a single strand. In other embodiments, the napDNAbp can be selected from the group consisting of: Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute and optionally has a nickase activity such that only one strand is cut. In some embodiments, the napDNAbp is selected from Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute and optionally has a nickase activity such that one DNA strand is cut preferentially to the other DNA strand.

### Nuclear Localization Sequence (NLS)

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO 2001/038547 on May 31, 2001, the contents of which are incorporated herein by reference for its disclosure of exemplary nuclear localization sequences. In some embodiments, an NLS is included in a fusion protein (e.g., in a prime editor as described herein). In certain embodiments, an NLS comprises the amino acid sequence PKKKRKV (SEQ ID NO: 94), MDSLLMNRRKFLYQFKNVRWAKGRRETYLC (SEQ ID NO: 99), KRTADGSEFESPKKKRKV (SEQ ID NO: 97), KRTADGSEFEPKKKRKV (SEQ ID NO: 106), NLSKRPAAIKKAGQAKKKK (SEQ ID NO: 107), PAAKRVKLD (SEQ ID NO: 98), RQRRNELKRSF (SEQ ID NO: 108), or NQSSNFGPMKGGNFGGRSSGPYGGGGQYFAKPRNQGGY (SEQ ID NO: 109).

### Nucleic Acid

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7-deazaadenosine, 7-deazaguanosine, 8-oxoadenosine, 8-oxoguanosine, O(6)-methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages). In some embodiments, a nucleic acid is a pegRNA or an epegRNA. In some embodiments, a nucleic acid is a target nucleic acid to be editing, e.g., in a genome.

### PEgRNA

As used herein, the terms “prime editing guide RNA” or “PEgRNA” or “pegRNA” or “extended guide RNA” refer to a specialized form of a guide RNA that has been modified to include one or more additional sequences for implementing the prime editing as described herein. As described herein, the prime editing guide RNAs comprise one or more “extended regions,” also referred to herein as “extension arms,” of nucleic acid sequence. The extended regions may comprise, but are not limited to, single-stranded RNA or DNA. Further, the extended regions may occur at the 3′ end of a traditional guide RNA. In other arrangements, the extended regions may occur at the 5′ end of a traditional guide RNA. In still other arrangements, the extended region may occur at an intramolecular region of the traditional guide RNA, for example, in the gRNA core region which associates and/or binds to the napDNAbp. The extended region comprises a “DNA synthesis template” or “reverse transcriptase template” that encodes (by the polymerase/reverse transcriptase of the prime editor) a single-stranded DNA which, in turn, has been designed to be (a) homologous with the endogenous target DNA to be edited, and (b) which comprises at least one desired nucleotide change (e.g., a transition, a transversion, a deletion, or an insertion) to be introduced or integrated into the endogenous target DNA. The extended region may also comprise other functional sequence elements, such as, but not limited to, a “primer binding site” and a “linker” sequence, or other structural elements, such as, but not limited to, aptamers, stem loops, hairpins, toe-loops (e.g., a 3′ toeloop), or an RNA-protein recruitment domain (e.g., MS2 hairpin). As used herein, the “primer binding site” comprises a sequence that hybridizes to a single-strand DNA sequence having a 3′ end generated from the nicked DNA of the R-loop.

In certain embodiments, the pegRNAs have a 3′ extension arm, a spacer, and a gRNA core. The 3′ extension arm further comprises in the 5′ to 3′ direction a DNA synthesis template, a primer binding site, and a linker. The DNA synthesis template may also be referred to more broadly as the “DNA synthesis template” where the polymerase of a prime editor described herein is not an RT, but another type of polymerase.

In certain other embodiments, the pegRNAs have a 5′ extension arm, a spacer, and a gRNA core. The 5′ extension further comprises in the 5′ to 3′ direction a DNA synthesis template, a primer binding site, and a linker. The DNA synthesis template may also be referred to more broadly as the “DNA synthesis template” where the polymerase of a prime editor described herein is not an RT, but another type of polymerase.

In still other embodiments, the pegRNAs have in the 5′ to 3′ direction a spacer, a gRNA core, and an extension arm. The extension arm is at the 3′ end of the pegRNA. The extension arm further comprises in the 5′ to 3′ direction a homology arm, an edit template, and a primer binding site. The extension arm may also comprise an optional modifier region at the 3′ and 5′ ends, which may be the same sequences or different sequences. In addition, the 3′ end of the pegRNA may comprise a transcriptional terminator sequence. These sequence elements of the pegRNAs are further described and defined herein.

In still other embodiments, the pegRNAs have in the 5′ to 3′ direction an extension arm, a spacer, and a gRNA core. The extension arm is at the 5′ end of the pegRNA. The extension arm further comprises in the 3′ to 5′ direction a primer binding site, an edit template, and a homology arm. The extension arm may also comprise an optional modifier region at the 3′ and 5′ ends, which may be the same sequences or different sequences. The pegRNAs may also comprise a transcriptional terminator sequence at the 3′ end. These sequence elements of the pegRNAs are further described and defined herein.

In some embodiments, the spacer sequence of the pegRNA is about 10, about 11, about 12, about 13, about 14, about 15, about 16, about 17, about 18, about 19, about 20, about 21, about 22, about 23, about 24, or about 25 nucleotides in length. In certain embodiments, the spacer sequence of the pegRNA is about 20 nucleotides in length. In some embodiments, the prime binding site is about 4, about 5, about 6, about 7, about 8, about 9, about 10, about 11, about 12, about 13, about 14, about 15, about 16, or about 17 nucleotides in length. In some embodiments, the homology arm of the pegRNA is about 5, about 6, about 7, about 8, about 9, about 10, about 11, about 12, about 13, about 14, about 15, about 16, about 17, about 18, about 19, or about 20 nucleotides in length. In some embodiments, the DNA synthesis template is from about 5 to about 58 nucleotides in length, about 10 to about 16 nucleotides in length, or about 12 to about 17 nucleotides in length. In certain embodiments, the DNA synthesis template is less than 15 nucleotides in length.

In some embodiments, a pegRNA is an “engineered pegRNA” (“epegRNA”). Relative to a pegRNA, an epegRNA comprises an additional structured motif, for example, attached to its 3′ end. Such additional structured motifs may stabilize the pegRNA or otherwise prevent it from being degraded. Suitable structured motifs include, but are not limited to, toe-loops, hairpins, stem-loops, pseudoknots, aptamers, G-quadruplexes, tRNAs, riboswitches, and ribozymes. In some embodiments, a 3′ structured motif comprises evopreq1.

pegRNAs are further described, e.g., in International Patent Application No. PCT/US2020/023721, filed Mar. 19, 2020, which published as WO 2020/191239; International Patent Application No. PCT/US2021/031439, filed May 7, 2021, which published as WO 2021/226558; International Patent Application No. PCT/2021/052097, filed Sep. 24, 2021, which published as WO 2022/067130; International Patent Application No. PCT/US2022/012054, filed Jan. 11, 2022, which published as WO 2022/150790; International Patent Application No. PCT/US2022/078655, filed Oct. 25, 2022, which published as WO 2023/076898; and International Patent Application No. PCT/US2022/074628, filed Aug. 5, 2022, which published as WO 2023/015309; the contents of each of which is incorporated by reference herein.

### PE1

As used herein, “PE1” refers to a prime editing composition comprising 1) a fusion protein comprising a Cas9 protein variant Cas9(H840A) and a wild type MMLV RT having the following structure: [NLS]-[Cas9(H840A)]-[linker]-[MMLV_RT(wt)]-NLS and 2) a desired PEgRNA, wherein the fusion protein (referred to as the PE1 protein) has the amino acid sequence of SEQ ID NO: 3, which is shown as follows.

(SEQ ID NO: 3)
MKRTADGSEFESPKKKRKVDKKYSIGLDIGTNSVGWAVITDEYKVPSKKFKVLG
NTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYID
GGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEIS
GVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQTVKV
VDELVKVMGRHKPENIVIEMARENQTTQKGQKNSRERMKRIEEGIKELGSQIL
KEHPVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTIDRKRYT
STKEVLDATLIHQSITGLYETRIDLSQLGGDSGGSSGGSSGSETPGTSESATPESS
GGSSGGSSTLNIEDEYRLHETSKEPDVSLGSTWLSDFPQAWAETGGMGLAVRQAPLIIPL
KATSTPVSIKQYPMSQEARLGIKPHIQRLLDQGILVPCQSPWNTPLLPVKKPGTNDYRPVQ
DLREVNKRVEDIHPTVPNPYNLLSGLPPSHQWYTVLDLKDAFFCLRLHPTSQPLFAFEWR
DPEMGISGQLTWTRLPQGFKNSPTLFDEALHRDLADFRIQHPDLILLQYVDDLLLAATSEL
DCQQGTRALLQTLGNLGYRASAKKAQICQKQVKYLGYLLKEGQRWLTEARKETVMGQPT
PKTPRQLREFLGTAGFCRLWIPGFAEMAAPLYPLTKTGTLFNWGPDQQKAYQEIKQALLT
APALGLPDLTKPFELFVDEKQGYAKGVLTQKLGPWRRPVAYLSKKLDPVAAGWPPCLRM
VAAIAVLTKDAGKLTMGQPLVILAPHAVEALVKQPPDRWLSNARMTHYQALLLDTDRVQF
GPVVALNPATLLPLPEEGLQHNCLDILAEAHGTRPDLTDQPLPDADHTWYTDGSSLLQEG
QRKAGAAVTTETEVIWAKALPAGTSAQRAELIALTQALKMAEGKKLNVYTDSRYAFATAHI
HGEIYRRRGLLTSEGKEIKNKDEILALLKALFLPKRLSIIHCPGHQKGHSAEARGNRMADQ
AARKAAITETPDTSTLLIENSSPSGGSKRTADGSEFEPKKKRKV
KEY:
NUCLEAR LOCALIZATION SEQUENCE (NLS) TOP: (SEQ ID NO: 95),
BOTTOM: (SEQ ID NO: 96)
CAS9(H840A) (SEQ ID NO: 10)
33-AMINO ACID LINKER (SEQ ID NO: 80)
M-MLV reverse transcriptase (SEQ ID NO: 30).

### PE2

As used herein, “PE2” refers to a prime editing composition comprising 1) a fusion protein comprising a Cas9 protein variant Cas9(H840A) and a variant MMLV RT having the following structure: [NLS]-[Cas9(H840A)]-[linker]-[MMLV_RT(D200N)(T330P)(L603W)(T306K)(W313F)]-NLS and 2) a desired PEgRNA, wherein the fusion protein (referred to as the PE2 protein) has the amino acid sequence of SEQ ID NO: 4, which is shown as follows:

(SEQ ID NO: 4)
MKRTADGSEFESPKKKRKVDKKYSIGLDIGTNSVGWAVITDEYKVPSKKFKVLG
NTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYID
GGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEIS
GVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQTVKV
VDELVKVMGRHKPENIVIEMARENQTTQKGQKNSRERMKRIEEGIKELGSQIL
KEHPVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTIDRKRYT
STKEVLDATLIHQSITGLYETRIDLSQLGGDSGGSSGGSSGSETPGTSESATPESS
GGSSGGSSTLNIEDEYRLHETSKEPDVSLGSTWLSDFPQAWAETGGMGLAVRQAPLIIPL
KATSTPVSIKQYPMSQEARLGIKPHIQRLLDQGILVPCQSPWNTPLLPVKKPGTNDYRPVQ
DLREVNKRVEDIHPTVPNPYNLLSGLPPSHQWYTVLDLKDAFFCLRLHPTSQPLFAFEWR
DPEMGISGQLTWTRLPQGFKNSPTLFNEALHRDLADFRIQHPDLILLQYVDDLLLAATSEL
DCQQGTRALLQTLGNLGYRASAKKAQICQKQVKYLGYLLKEGQRWLTEARKETVMGQPT
PKTPRQLREFLGKAGFCRLFIPGFAEMAAPLYPLTKPGTLFNWGPDQQKAYQEIKQALLT
APALGLPDLTKPFELFVDEKQGYAKGVLTQKLGPWRRPVAYLSKKLDPVAAGWPPCLRM
VAAIAVLTKDAGKLTMGQPLVILAPHAVEALVKQPPDRWLSNARMTHYQALLLDTDRVQF
GPVVALNPATLLPLPEEGLQHNCLDILAEAHGTRPDLTDQPLPDADHTWYTDGSSLLQEG
QRKAGAAVTTETEVIWAKALPAGTSAQRAELIALTQALKMAEGKKLNVYTDSRYAFATAHI
HGEIYRRRGWLTSEGKEIKNKDEILALLKALFLPKRLSIIHCPGHQKGHSAEARGNRMADQ
AARKAAITETPDTSTLLIENSSPSGGSKRTADGSEFEPKKKRKV
KEY:
NUCLEAR LOCALIZATION SEQUENCE (NLS) TOP: (SEQ ID NO: 95),
BOTTOM: (SEQ ID NO: 96)
CAS9(H840A) (SEQ ID NO: 10)
33-AMINO ACID LINKER (SEQ ID NO: 80)
M-MLV reverse transcriptase (SEQ ID NO: 29).

### PE3

As used herein, “PE3” refers to a prime editing composition comprising a PE2 prime editor and further comprising a second-strand nicking guide RNA that complexes with PE2 and introduces a nick in the non-edit DNA strand in order to induce preferential replacement of the edit strand.

### PE3b

As used herein, “PE3b” refers to a prime editing composition comprising PE2 and further comprising a second-strand nicking guide RNA that complexes with PE2 and introduces a nick in the non-edit DNA strand, wherein the second-strand nicking guide RNA is designed for temporal control such that the second strand nick is not introduced until after the installation of the desired edit. This is achieved by designing the second strand nicking guide RNA with a spacer sequence that comprises complementarity to, and only hybridizes with, the edited strand after installation of the desired nucleotide edit(s), but not the endogenous target DNA sequence. Using this strategy, mismatches between the nicking guide RNA spacer and the unedited target DNA should disfavor nicking by the sgRNA until after the editing event on the PAM strand takes place.

### PE4

As used herein, “PE4” refers to a prime editing composition comprising a PE2 and further comprising an MLH1 dominant negative protein variant (i.e., wild-type MLH1 with amino acids 754-756 truncated, which may be referred to herein as “MLH1 Δ754-756” or “MLH1dn”). The MLH1 dominant negative protein variant may be expressed in trans in some embodiments. In some embodiments, a PE4 system comprises a fusion protein comprising a PE2 protein and an MLH1 dominant negative protein joined via an optional linker.

### PE5 and PE5b

As used herein, “PE5” refers to a prime editing composition comprising a PE3 prime editor and further comprising an MLH1 dominant negative protein variant (i.e., wild-type MLH1 with amino acids 754-756 truncated, which may be referred to as “MLH1 Δ754-756” or “MLH1dn”). The MLH1 dominant negative variant may be expressed in trans in some embodiments. In some embodiments, a PE5 system comprises a fusion protein comprising a PE2 protein and an MLH1 dominant negative protein joined via an optional linker. “PE5b” refers to a prime editing composition comprising a PE3 and an MLH1 dominant negative protein, wherein the second-strand nicking guide RNA is designed for temporal control such that the second strand nick is not introduced until after the installation of the desired edit. This is achieved by designing the second strand nicking guide RNA with a spacer sequence that comprise complementarity to, and hybridize with, only the edited strand after installation of the desired nucleotide edit(s), but not the endogenous target DNA sequence.

### PE6

The term “PE6” refers to a suite of next-generation prime editors described herein (PE6a, PE6b, PE6c, PE6d, PE6e, PE6f, and PE6g) comprising improved reverse transcriptase and/or Cas9 variants. The improved reverse transcriptase and Cas9 domains of the PE6 variants can also be combined with each other to offer cumulative benefits. For example, a PE6 prime editor comprising an improved reverse transcriptase variant of PE6a and an improved Cas9 variant of PE6e is referred to herein as the prime editor “PE6a-e” (or “PE6e-a”). Any possible combination of PE6 prime editors is contemplated by the present disclosure including, for example, PE6a-e, PE6a-f, PE6a-g, PE6b-e, PE6b-f, PE6b-g, PE6c-e, PE6c-f, PE6c-g, PE6d-e, PE6d-f, and PE6d-g.

Each of the PE6 prime editors comprise a Cas9 domain, e.g., a Cas9 variant, and a reverse transcriptase domain, e.g., a reverse transcriptase variant. PE6a comprises a reverse transcriptase variant comprising the amino acid substitutions E60K, K87E, E165D, D243N, R267I, E279K, K318E, and K343N relative to an Ec48 reverse transcriptase (SEQ ID NO: 7). PE6b comprises a reverse transcriptase variant comprising the amino acid substitutions P70T, G72V, S87G, M102I, K106R, K118R, I128V, L158Q, F269L, A363V, K413E, and S492N relative to a Tf1 reverse transcriptase (SEQ ID NO: 1). PE6c comprises a reverse transcriptase variant comprising the amino acid substitutions P70T, G72V, S87G, M102I, K106R, K118R, I128V, L158Q, S188K, I260L, F269L, R288Q, S297Q, A363V, K413E, and S492N relative to a Tf1 reverse transcriptase (SEQ ID NO: 1). PE6d comprises a reverse transcriptase variant comprising the amino acid substitutions T128N, D200C, and V223Y (and the substitutions T306K, W313F, and T330P used in the MMLV reverse transcriptase of PE2 and PEmax) relative to a MMLV reverse transcriptase (SEQ ID NO: 30) with a truncation of the C-terminal RNaseH domain (e.g., between D497 and I498 of SEQ ID NO: 30). PE6e comprises a Cas9 variant comprising the amino acid substitutions K775R and K918A relative to wild type Streptococcus pyogenes Cas9 or Streptococcus pyogenes Cas9 nickase (SEQ ID NO: 2). PE6f comprises a Cas9 variant comprising the amino acid substitutions H99R, E471K, I632V, D645N, H721Y, and K918A relative to wild type Streptococcus pyogenes Cas9 or Streptococcus pyogenes Cas9 nickase (SEQ ID NO: 2). PE6g comprises a Cas9 variant comprising the amino acid substitutions H99R, E471K, I632V, D645N, R654C, and H721Y relative to wild type Streptococcus pyogenes Cas9 or Streptococcus pyogenes Cas9 nickase (SEQ ID NO: 2). The Cas9 domain and the reverse transcriptase variant of a PE6 prime editor described herein can be covalently linked or associated, for example, directly fused or connected to each other via a linker peptide to form a fusion protein. Alternatively, the Cas9 domain and the reverse transcriptase may be provided in trans, i.e., not covalently connected. Any of the PE6 prime editor fusion proteins provided herein may also comprise the architecture of the prime editor fusion proteins described herein or known in the art, for example, a PE2 protein architecture or a PEmax protein architecture. Components, sequences, and corresponding architecture of an exemplary PEmax protein is provided below. In some embodiments, any of the PE6 prime editors provided herein may further comprise additional amino acid mutations, e.g., any of those included in PEmax as provided below.

In some embodiments, a PE6 protein comprises a reverse transcriptase variant disclosed herein and a Cas9 protein that recognizes a non-canonical PAM sequence (e.g., a Cas9 protein of SEQ ID NO: 133, or at least 80%, at least 85%, at least 90%, at least 95%, at least 96%, at least 97%, at least 98%, or at least 99% identical to the amino acid sequence of SEQ ID NO: 133). For example, the prime editor “PE6b-NRCH” comprises the reverse transcriptase of PE6b (SEQ ID NO: 25) and the NRCH-Cas9 protein of SEQ ID NO: 133. PE6b-NRCH comprises the amino acid sequence:

(SEQ ID NO: 155)
MKRTADGSEFESPKKKRKVDKKYSIGLDIGTNSVGWAVITDEYKVPSKKFKVLGNT
DRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAIL
SARLSKSRKLENLIAQLPGEKKNGLFGNLIALSLGLTPNFKSNFDLAEDAKLQLSKDT
YDDDLDNLLAQIGDQYADLFLAAKNLSDAILLSDILRVNTEITKAPLSASMVKRYDE
HHQDLTLLKALVRQQLPEKYKEIFFDQSKNGYAGYIDGGASQEEFYKFIKPILEKMD
GTEELLVKLKREDLLRKQRTFDNGIIPHQIHLGELHAILRRQGDFYPFLKDNREKIEKI
LTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTN
RKVTVKQLKEDYFKKIECFDSVEISGVEDRFNASLGTYHDLLKIIKDKDFLDNEENED
ILEDIVLTLTLFEDREMIEERLKTYAHLFDDKVMKQLKRLRYTGWGRLSRKLINGIRD
KQSGKTILDFLKSDGFANRNFMQLIHDDSLTFKEDIQKAQVSGQGDSLHEHIANLAG
SPAIKKGILQTVKVVDELVKVMGGHKPENIVIEMARENQTTQKGQKNSRERMKRIEE
GIKELGSQILKEHPVENTQLQNEKLYLYYLQNGRDMYVDQELDINRLSDYDVDAIVP
QSFLKDDSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKGNSDKLIARKKDWDP
KKYGGFNSPTVAYSVLVVAKVEKGKSKKLKSVKELLGITIMERSSFEKNPIDFLEAK
GYKEVKKDLIIKLPKYSLFELENGRKRMLASAGVLQKGNELALPSKYVNFLYLASHY
EKLKGSPEDNEQKQLFVEQHKHYLDEIIEQISEFSKRVILADANLDKVLSAYNKHRD
KPIREQAENIIHLFTLTNLGAPAAFKYFDTTINRKQYNTTKEVLDATLIRQSITGLYET
RIDLSQLGGDSGGSSGGSKRTADGSEFESPKKKRKVSGGSSGGSISSSKHTLSQMNKV
SNIVKEPELPDIYKEFKDITADTNTEKLPKPIKGLEFEVELTQENYRLPIRNYPLTPVK
MQAMNDEINQGLKGGIIRESKAINACPVIFVPRKEGTLRMVVDYRPLNKYVKPNVYP
LPLIEQLLAKIQGSTIFTKLDLKSAYHQIRVRKGDEHKLAFRCPRGVFEYLVMPYGIST
APAHFQYFINTILGEAKESHVVCYMDDILIHSKSESEHVKHVKDVLQKLKNANLIINQ
AKCEFHQSQVKFIGYHISEKGLTPCQENIDKVLQWKQPKNRKELRQFLGSVNYLRKF
IPKTSQLTHPLNKLLKKDVRWKWTPTQTQAIENIKQCLVSPPVLRHFDFSKKILLETD
VSDVAVGAVLSQKHDDDKYYPVGYYSAKMSKAQLNYSVSDKEMLAIIKSLEHWRH
YLESTIEPFKILTDHRNLIGRITNESEPENKRLARWQLFLQDFNFEINYRPGSANHIAD
ALSRIVDETEPIPKDNEDNSINFVNQISIKRTADGSEFESPKKKRKVPAAKRVKLD.

The prime editor “PE6c-NRCH” comprises the reverse transcriptase of PE6c (SEQ ID NO: 26) and the NRCH-Cas9 protein of SEQ ID NO: 133. PE6c-NRCH comprises the amino acid sequence:

(SEQ ID NO: 156)
MKRTADGSEFESPKKKRKVDKKYSIGLDIGTNSVGWAVITDEYKVPSKKFKVLGNT
DRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAIL
SARLSKSRKLENLIAQLPGEKKNGLFGNLIALSLGLTPNFKSNFDLAEDAKLQLSKDT
YDDDLDNLLAQIGDQYADLFLAAKNLSDAILLSDILRVNTEITKAPLSASMVKRYDE
HHQDLTLLKALVRQQLPEKYKEIFFDQSKNGYAGYIDGGASQEEFYKFIKPILEKMD
GTEELLVKLKREDLLRKQRTFDNGIIPHQIHLGELHAILRRQGDFYPFLKDNREKIEKI
LTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTN
RKVTVKQLKEDYFKKIECFDSVEISGVEDRFNASLGTYHDLLKIIKDKDFLDNEENED
ILEDIVLTLTLFEDREMIEERLKTYAHLFDDKVMKQLKRLRYTGWGRLSRKLINGIRD
KQSGKTILDFLKSDGFANRNFMQLIHDDSLTFKEDIQKAQVSGQGDSLHEHIANLAG
SPAIKKGILQTVKVVDELVKVMGGHKPENIVIEMARENQTTQKGQKNSRERMKRIEE
GIKELGSQILKEHPVENTQLQNEKLYLYYLQNGRDMYVDQELDINRLSDYDVDAIVP
QSFLKDDSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKGNSDKLIARKKDWDP
KKYGGFNSPTVAYSVLVVAKVEKGKSKKLKSVKELLGITIMERSSFEKNPIDFLEAK
GYKEVKKDLIIKLPKYSLFELENGRKRMLASAGVLQKGNELALPSKYVNFLYLASHY
EKLKGSPEDNEQKQLFVEQHKHYLDEIIEQISEFSKRVILADANLDKVLSAYNKHRD
KPIREQAENIIHLFTLTNLGAPAAFKYFDTTINRKQYNTTKEVLDATLIRQSITGLYET
RIDLSQLGGDSGGSSGGSKRTADGSEFESPKKKRKVSGGSSGGSISSSKHTLSQMNKV
SNIVKEPELPDIYKEFKDITADTNTEKLPKPIKGLEFEVELTQENYRLPIRNYPLTPVK
MQAMNDEINQGLKGGIIRESKAINACPVIFVPRKEGTLRMVVDYRPLNKYVKPNVYP
LPLIEQLLAKIQGSTIFTKLDLKSAYHQIRVRKGDEHKLAFRCPRGVFEYLVMPYGIK
TAPAHFQYFINTILGEAKESHVVCYMDDILIHSKSESEHVKHVKDVLQKLKNANLIIN
QAKCEFHQSQVKFLGYHISEKGLTPCQENIDKVLQWKQPKNQKELRQFLGQVNYLR
KFIPKTSQLTHPLNKLLKKDVRWKWTPTQTQAIENIKQCLVSPPVLRHFDFSKKILLE
TDVSDVAVGAVLSQKHDDDKYYPVGYYSAKMSKAQLNYSVSDKEMLAIIKSLEHW
RHYLESTIEPFKILTDHRNLIGRITNESEPENKRLARWQLFLQDFNFEINYRPGSANHIA
DALSRIVDETEPIPKDNEDNSINFVNQISIKRTADGSEFESPKKKRKVPAAKRVKLD.

The prime editor “PE6d-NRCH” comprises the reverse transcriptase of PE6d (SEQ ID NO: 27) and the Cas9 variant as set forth in SEQ ID NO: 133. In some embodiments, a prime editor fusion protein comprises a non-truncated version of the MMLV reverse transcriptase variant having amino acid substitutions T128N, D200C, V223Y, T306K, W313F and T330P relative to SEQ ID NO: 30, and the NRCH-Cas9 protein of SEQ ID NO: 133. PE6d-NRCH comprises the amino acid sequence:

(SEQ ID NO: 157)
MKRTADGSEFESPKKKRKVDKKYSIGLDIGTNSVGWAVITDEYKVPSKKFKVLGNT
DRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAIL
SARLSKSRKLENLIAQLPGEKKNGLFGNLIALSLGLTPNFKSNFDLAEDAKLQLSKDT
YDDDLDNLLAQIGDQYADLFLAAKNLSDAILLSDILRVNTEITKAPLSASMVKRYDE
HHQDLTLLKALVRQQLPEKYKEIFFDQSKNGYAGYIDGGASQEEFYKFIKPILEKMD
GTEELLVKLKREDLLRKQRTFDNGIIPHQIHLGELHAILRRQGDFYPFLKDNREKIEKI
LTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTN
RKVTVKQLKEDYFKKIECFDSVEISGVEDRFNASLGTYHDLLKIIKDKDFLDNEENED
ILEDIVLTLTLFEDREMIEERLKTYAHLFDDKVMKQLKRLRYTGWGRLSRKLINGIRD
KQSGKTILDFLKSDGFANRNFMQLIHDDSLTFKEDIQKAQVSGQGDSLHEHIANLAG
SPAIKKGILQTVKVVDELVKVMGGHKPENIVIEMARENQTTQKGQKNSRERMKRIEE
GIKELGSQILKEHPVENTQLQNEKLYLYYLQNGRDMYVDQELDINRLSDYDVDAIVP
QSFLKDDSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKGNSDKLIARKKDWDP
KKYGGFNSPTVAYSVLVVAKVEKGKSKKLKSVKELLGITIMERSSFEKNPIDFLEAK
GYKEVKKDLIIKLPKYSLFELENGRKRMLASAGVLQKGNELALPSKYVNFLYLASHY
EKLKGSPEDNEQKQLFVEQHKHYLDEIIEQISEFSKRVILADANLDKVLSAYNKHRD
KPIREQAENIIHLFTLTNLGAPAAFKYFDTTINRKQYNTTKEVLDATLIRQSITGLYET
RIDLSQLGGDSGGSSGGSKRTADGSEFESPKKKRKVSGGSSGGSTLNIEDEYRLHETS
KEPDVSLGSTWLSDFPQAWAETGGMGLAVRQAPLIIPLKATSTPVSIKQYPMSQEAR
LGIKPHIQRLLDQGILVPCQSPWNTPLLPVKKPGTNDYRPVQDLREVNKRVEDIHPNV
PNPYNLLSGLPPSHQWYTVLDLKDAFFCLRLHPTSQPLFAFEWRDPEMGISGQLTWT
RLPQGFKNSPTLFCEALHRDLADFRIQHPDLILLQYYDDLLLAATSELDCQQGTRALL
QTLGNLGYRASAKKAQICQKQVKYLGYLLKEGQRWLTEARKETVMGQPTPKTPRQ
LREFLGKAGFCRLFIPGFAEMAAPLYPLTKPGTLFNWGPDQQKAYQEIKQALLTAPA
LGLPDLTKPFELFVDEKQGYAKGVLTQKLGPWRRPVAYLSKKLDPVAAGWPPCLR
MVAAIAVLTKDAGKLTMGQPLVILAPHAVEALVKQPPDRWLSNARMTHYQALLLD
TDRVQFGPVVALNPATLLPLPEEGLQHNCLDSGGSKRTADGSEFESPKKKRKVPAAK
RVKLD.

An exemplary prime editor fusion protein comprising a non-truncated version of the MMLV reverse transcriptase variant having amino acid substitutions T128N, D200C, V223Y, T306K, W313F, T330P, and L603W relative to SEQ ID NO: 30 and the NRCH-Cas9 protein of SEQ ID NO: 133 (which has a full-length MMLV reverse transcriptase domain relative to SEQ ID NO: 30 as described herein) can have the amino acid sequence:

(SEQ ID NO: 158)
MKRTADGSEFESPKKKRKVDKKYSIGLDIGTNSVGWAVITDEYKVPSKKFKVLGNT
DRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAIL
SARLSKSRKLENLIAQLPGEKKNGLFGNLIALSLGLTPNFKSNFDLAEDAKLQLSKDT
YDDDLDNLLAQIGDQYADLFLAAKNLSDAILLSDILRVNTEITKAPLSASMVKRYDE
HHQDLTLLKALVRQQLPEKYKEIFFDQSKNGYAGYIDGGASQEEFYKFIKPILEKMD
GTEELLVKLKREDLLRKQRTFDNGIIPHQIHLGELHAILRRQGDFYPFLKDNREKIEKI
LTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTN
RKVTVKQLKEDYFKKIECFDSVEISGVEDRFNASLGTYHDLLKIIKDKDFLDNEENED
ILEDIVLTLTLFEDREMIEERLKTYAHLFDDKVMKQLKRLRYTGWGRLSRKLINGIRD
KQSGKTILDFLKSDGFANRNFMQLIHDDSLTFKEDIQKAQVSGQGDSLHEHIANLAG
SPAIKKGILQTVKVVDELVKVMGGHKPENIVIEMARENQTTQKGQKNSRERMKRIEE
GIKELGSQILKEHPVENTQLQNEKLYLYYLQNGRDMYVDQELDINRLSDYDVDAIVP
QSFLKDDSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKGNSDKLIARKKDWDP
KKYGGFNSPTVAYSVLVVAKVEKGKSKKLKSVKELLGITIMERSSFEKNPIDFLEAK
GYKEVKKDLIIKLPKYSLFELENGRKRMLASAGVLQKGNELALPSKYVNFLYLASHY
EKLKGSPEDNEQKQLFVEQHKHYLDEIIEQISEFSKRVILADANLDKVLSAYNKHRD
KPIREQAENIIHLFTLTNLGAPAAFKYFDTTINRKQYNTTKEVLDATLIRQSITGLYET
RIDLSQLGGDSGGSSGGSKRTADGSEFESPKKKRKVSGGSSGGSTLNIEDEYRLHETS
KEPDVSLGSTWLSDFPQAWAETGGMGLAVRQAPLIIPLKATSTPVSIKQYPMSQEAR
LGIKPHIQRLLDQGILVPCQSPWNTPLLPVKKPGTNDYRPVQDLREVNKRVEDIHPNV
PNPYNLLSGLPPSHQWYTVLDLKDAFFCLRLHPTSQPLFAFEWRDPEMGISGQLTWT
RLPQGFKNSPTLFCEALHRDLADFRIQHPDLILLQYYDDLLLAATSELDCQQGTRALL
QTLGNLGYRASAKKAQICQKQVKYLGYLLKEGQRWLTEARKETVMGQPTPKTPRQ
LREFLGKAGFCRLFIPGFAEMAAPLYPLTKPGTLFNWGPDQQKAYQEIKQALLTAPA
LGLPDLTKPFELFVDEKQGYAKGVLTQKLGPWRRPVAYLSKKLDPVAAGWPPCLR
MVAAIAVLTKDAGKLTMGQPLVILAPHAVEALVKQPPDRWLSNARMTHYQALLLD
TDRVQFGPVVALNPATLLPLPEEGLQHNCLDILAEAHGTRPDLTDQPLPDADHTWYT
DGSSLLQEGQRKAGAAVTTETEVIWAKALPAGTSAQRAELIALTQALKMAEGKKLN
VYTDSRYAFATAHIHGEIYRRRGWLTSEGKEIKNKDEILALLKALFLPKRLSIIHCPGH
QKGHSAEARGNRMADQAARKAAITETPDTSTLLIENSSPSGGSKRTADGSEFESPKK
KRKVPAAKRVKLD.

In some embodiments, a PE6d prime editor comprises a fusion protein that comprises a Cas9 having amino acid substitutions R221K, N394K, and relative to SEQ ID NO: 2 (i.e., R221K, N394K, and H840A substitutions relative to a wildtype Cas9) and a MMLV-RT having amino acid substitutions T128N, D200C, V223Y, T306K, W313F, and T330P, and a C terminal truncation between D497 and I498, relative to SEQ ID NO: 30. In some embodiments, a PE6d prime editor comprises a fusion protein comprising the reverse transcriptase variant as set forth in SEQ ID NO: 27 and the Cas9 variant as set forth in SEQ ID NO: 11. In some embodiments, a PE6d prime editor comprises a fusion protein having the following configuration (the “PEmax architecture”): [bipartite NLS]-[Cas9(R221K)(N394K)(H840A)]-[linker]-[MMLV_RT(T128N)(D200C)(V223Y)(T306K)(T330P)(D497/I498 C-term truncation))]-[bipartite NLS]-[NLS]. In some embodiments, a PE6d prime editor comprises a fusion protein having the following sequence:

(SEQ ID NO: 159)
MKRTADGSEFESPKKKRKVDKKYSIGLDIGTNSVGWAVITDEYKVPSKKFKVLG
NTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPI
NASGVDAKAILSARLSKSRKLENLIAQLPGEKKNGLFGNLIALSLGLTPNFKSNF
DLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYID
GGASQEEFYKFIKPILEKMDGTEELLVKLKREDLLRKQRTFDNGSIPHQIHLGE
LHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEIS
GVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQTVKV
VDELVKVMGRHKPENIVIEMARENQTTQKGQKNSRERMKRIEEGIKELGSQIL
KEHPVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTIDRKRYT
STKEVLDATLIHQSITGLYETRIDLSQLGGDSGGSSGGSKRTADGSEFESPKKKR
KVSGGSSGGSTLNIEDEYRLHETSKEPDVSLGSTWLSDFPQAWAETGGMGLAVRQAPLII
PLKATSTPVSIKQYPMSQEARLGIKPHIQRLLDQGILVPCQSPWNTPLLPVKKPGTNDYRP
VQDLREVNKRVEDIHPNVPNPYNLLSGLPPSHQWYTVLDLKDAFFCLRLHPTSQPLFAFE
WRDPEMGISGQLTWTRLPQGFKNSPTLFCEALHRDLADFRIQHPDLILLQYYDDLLLAAT
SELDCQQGTRALLQTLGNLGYRASAKKAQICQKQVKYLGYLLKEGQRWLTEARKETVMG
QPTPKTPRQLREFLGKAGFCRLFIPGFAEMAAPLYPLTKPGTLFNWGPDQQKAYQEIKQ
ALLTAPALGLPDLTKPFELFVDEKQGYAKGVLTQKLGPWRRPVAYLSKKLDPVAAGWPP
CLRMVAAIAVLTKDAGKLTMGQPLVILAPHAVEALVKQPPDRWLSNARMTHYQALLLDTD
RVQFGPVVALNPATLLPLPEEGLQHNCLDSGGSKRTADGSEFESPKKKRKVGSGPAAKR
VKLD
KEY:
BIPARTITE SV40 NUCLEAR LOCALIZATION SEQUENCE (NLS) TOP:
(SEQ ID NO: 95),
CAS9(R221K N394K H840A) (SEQ ID NO: 11)
SGGSx2-BIPARTITE SV40NLS-SGGSx2 LINKER (SEQ ID NO: 79)
M-MLV reverse transcriptase(T128N D200C V223Y T306K W313F
T330PD497/1498truncation ) (SEQ ID NO: 27)
Other linker sequence (SEQ ID NO: 82)
BIPARTITE SV40NLS (SEQ ID NO: 97)
Other linker sequence (GSG)
c-Myc NLS (SEQ ID NO: 98)

In some embodiments, a PE6d prime editor comprises a fusion protein that comprises a Cas9 comprising an amino acid sequence as set forth in SEQ ID NO: 10 (i.e., H840A substitutions relative to a wildtype Cas9) and a MMLV-RT having amino acid substitutions T128N, D200C, V223Y, T306K, W313F, and T330P, and a C terminal truncation between D497 and I498, relative to SEQ ID NO: 30. In some embodiments, a PE6d prime editor comprises a fusion protein comprising the reverse transcriptase as set forth in SEQ ID NO: 27 and the Cas9 variant as set forth in SEQ ID NO: 10. In some embodiments, a PE6d prime editor comprises a fusion protein having the following configuration (the “PE2 architecture”): [bipartite NLS]-[Cas9 (H840A)]-[linker]-[MMLV_RT(T128N)(D200C)(V223Y)(T306K)(T330P)(D497/I498 C-term truncation))]-[NLS]. In some embodiments, a PE6d prime editor comprises a fusion protein having the following sequence:

(SEQ ID NO: 160)
MKRTADGSEFESPKKKRKVDKKYSIGLDIGTNSVGWAVITDEYKVPSKKFKVLG
NTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYID
GGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEIS
GVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQTVKV
VDELVKVMGRHKPENIVIEMARENQTTQKGQKNSRERMKRIEEGIKELGSQIL
KEHPVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTIDRKRYT
STKEVLDATLIHQSITGLYETRIDLSQLGGDSGGSSGGSSGSETPGTSESATPESS
GGSSGGSSTLNIEDEYRLHETSKEPDVSLGSTWLSDFPQAWAETGGMGLAVRQAPLIIPL
KATSTPVSIKQYPMSQEARLGIKPHIQRLLDQGILVPCQSPWNTPLLPVKKPGTNDYRPVQ
DLREVNKRVEDIHPNVPNPYNLLSGLPPSHQWYTVLDLKDAFFCLRLHPTSQPLFAFEWR
DPEMGISGQLTWTRLPQGFKNSPTLFCEALHRDLADFRIQHPDLILLQYYDDLLLAATSEL
DCQQGTRALLQTLGNLGYRASAKKAQICQKQVKYLGYLLKEGQRWLTEARKETVMGQPT
PKTPRQLREFLGKAGFCRLFIPGFAEMAAPLYPLTKPGTLFNWGPDQQKAYQEIKQALLT
APALGLPDLTKPFELFVDEKQGYAKGVLTQKLGPWRRPVAYLSKKLDPVAAGWPPCLRM
VAAIAVLTKDAGKLTMGQPLVILAPHAVEALVKQPPDRWLSNARMTHYQALLLDTDRVQF
GPVVALNPATLLPLPEEGLQHNCLDSGGSKRTADGSEFEPKKKRKV
KEY:
NUCLEAR LOCALIZATION SEQUENCE (NLS) TOP:(SEQ ID NO: 95), BOTTOM:
(SEQ ID NO: 96)
CAS9(H840A) (SEQ ID NO: 10)
33-AMINO ACID LINKER (SEQ ID NO: 80)
M-MLV reverse transcriptase (SEQ ID NO: 27).

### PE7

The term “PE7” refers to the PE6 prime editors plus a second strand nicking guide RNA. For example, “PE7a” refers to the PE6a prime editor as provided herein, plus a second strand nicking guide RNA.

### PEmax

As used herein, “PEmax” refers to a prime editing composition comprising 1) a fusion protein comprising a Cas9 protein variant Cas9(R221K N394K H840A) and a variant MMLV RT having the following structure: [bipartite NLS]-[Cas9(R221K)(N394K)(H840A)]-[linker]-[MMLV_RT(D200N)(T306K)(W313F)(T330P)(L603W)]-[bipartite NLS]-[NLS] and 2) a desired PEgRNA, wherein the fusion protein (referred to as the PEmax protein) has the amino acid sequence of SEQ ID NO: 5, which is shown as follows:

(SEQ ID NO: 5)
MKRTADGSEFESPKKKRKVDKKYSIGLDIGTNSVGWAVITDEYKVPSKKFKVLG
NTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPI
NASGVDAKAILSARLSKSRKLENLIAQLPGEKKNGLFGNLIALSLGLTPNFKSNF
DLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYID
GGASQEEFYKFIKPILEKMDGTEELLVKLKREDLLRKQRTFDNGSIPHQIHLGE
LHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEIS
GVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQTVKV
VDELVKVMGRHKPENIVIEMARENQTTQKGQKNSRERMKRIEEGIKELGSQIL
KEHPVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTIDRKRYT
STKEVLDATLIHQSITGLYETRIDLSQLGGDSGGSSGGSKRTADGSEFESPKKKR
KVSGGSSGGSTLNIEDEYRLHETSKEPDVSLGSTWLSDFPQAWAETGGMGLAVRQA
PLIIPLKATSTPVSIKQYPMSQEARLGIKPHIQRLLDQGILVPCQSPWNTPLLPVKKPGT
NDYRPVQDLREVNKRVEDIHPTVPNPYNLLSGLPPSHQWYTVLDLKDAFFCLRLHPT
SQPLFAFEWRDPEMGISGQLTWTRLPQGFKNSPTLFNEALHRDLADFRIQHPDLILLQ
YVDDLLLAATSELDCQQGTRALLQTLGNLGYRASAKKAQICQKQVKYLGYLLKEG
QRWLTEARKETVMGQPTPKTPRQLREFLGKAGFCRLFIPGFAEMAAPLYPLTKPGTL
FNWGPDQQKAYQEIKQALLTAPALGLPDLTKPFELFVDEKQGYAKGVLTQKLGPWR
RPVAYLSKKLDPVAAGWPPCLRMVAAIAVLTKDAGKLTMGQPLVILAPHAVEALV
KQPPDRWLSNARMTHYQALLLDTDRVQFGPVVALNPATLLPLPEEGLQHNCLDILA
EAHGTRPDLTDQPLPDADHTWYTDGSSLLQEGQRKAGAAVTTETEVIWAKALPAGT
SAQRAELIALTQALKMAEGKKLNVYTDSRYAFATAHIHGEIYRRRGWLTSEGKEIKN
KDEILALLKALFLPKRLSIIHCPGHQKGHSAEARGNRMADQAARKAAITETPDTSTLL
IENSSPSGGSKRTADGSEFESPKKKRKVGSGPAAKRVKLD
KEY:
BIPARTITE SV40 NUCLEAR LOCALIZATION SEQUENCE (NLS) TOP:
(SEQ ID NO: 95),
CAS9(R221K N394K H840A) (SEQ ID NO: 11)
SGGSx2-BIPARTITE SV40NLS-SGGSx2 LINKER (SEQ ID NO: 79)
M-MLV reverse transcriptase(D200N T306K W313F T330P L603W)
(SEQ ID NO: 29)
Other linker sequence (SEQ ID NO: 82)
BIPARTITE SV40NLS (SEQ ID NO: 97)
Other linker sequence (GSG)
c-Myc NLS (SEQ ID NO: 98)

### PACE

The term “phage-assisted continuous evolution (PACE),” as used herein, refers to continuous evolution that employs phage as viral vectors. The general concept of PACE technology has been described, for example, in International PCT application, PCT/US2009/056194, filed Sep. 8, 2009, published as WO 2010/028347 on Mar. 11, 2010; International PCT application, PCT/US2011/066747, filed Dec. 22, 2011, published as WO 2012/088381 on Jun. 28, 2012; U.S. application, U.S. Pat. No. 9,023,594, issued May 5, 2015, International PCT application, PCT/US2015/012022, filed Jan. 20, 2015, published as WO 2015/134121 on Sep. 11, 2015, and International PCT application, PCT/US2016/027795, filed Apr. 15, 2016, published as WO 2016/168631 on Oct. 20, 2016, the entire contents of each of which are incorporated herein by reference.

### PANCE

Phage-assisted non-continuous evolution (PANCE),” as used herein, refers to non-continuous evolution that employs phage as viral vectors. PANCE is a simplified technique for rapid in vivo directed evolution using serial flask transfers of evolving “selection phage” (SP), which contain a gene of interest to be evolved, across fresh E. coli host cells, thereby allowing genes inside the host E. coli to be held constant while genes contained in the SP continuously evolve. Serial flask transfers have long served as a widely-accessible approach for laboratory evolution of microbes, and, more recently, analogous approaches have been developed for bacteriophage evolution. The PANCE system features lower stringency than the PACE system.

### Polymerase

As used herein, the term “polymerase” refers to an enzyme that synthesizes a nucleotide strand and that may be used in connection with the prime editor delivery systems described herein. The polymerase can be a “template-dependent” polymerase (i.e., a polymerase that synthesizes a nucleotide strand based on the order of nucleotide bases of a template strand). The polymerase can also be a “template-independent” polymerase (i.e., a polymerase that synthesizes a nucleotide strand without the requirement of a template strand). A polymerase may also be further categorized as a “DNA polymerase” or an “RNA polymerase.” In various embodiments, the prime editor system comprises a DNA polymerase. In various embodiments, the DNA polymerase can be a “DNA-dependent DNA polymerase” (i.e., whereby the template molecule is a strand of DNA). In such cases, the DNA template molecule can be a pegRNA, wherein the extension arm comprises a strand of DNA. In such cases, the pegRNA may be referred to as a chimeric or hybrid pegRNA that comprises an RNA portion (i.e., the guide RNA components, including the spacer and the gRNA core) and a DNA portion (i.e., the extension arm). In various other embodiments, the DNA polymerase can be an “RNA-dependent DNA polymerase” (i.e., whereby the template molecule is a strand of RNA). In such cases, the pegRNA is RNA, i.e., including an RNA extension. The term “polymerase” may also refer to an enzyme that catalyzes the polymerization of nucleotides (i.e., the polymerase activity). Generally, the enzyme will initiate synthesis at the 3′-end of a primer annealed to a polynucleotide template sequence (e.g., such as a primer sequence annealed to the primer binding site of a pegRNA) and will proceed toward the 5′ end of the template strand. A “DNA polymerase” catalyzes the polymerization of deoxynucleotides. As used herein in reference to a DNA polymerase, the term DNA polymerase includes a “functional fragment thereof.” A “functional fragment thereof” refers to any portion of a wild-type or mutant DNA polymerase that encompasses less than the entire amino acid sequence of the polymerase and which retains the ability, under at least one set of conditions, to catalyze the polymerization of a polynucleotide. Such a functional fragment may exist as a separate entity, or it may be a constituent of a larger polypeptide, such as a fusion protein.

### Prime Editing

As used herein, the term “prime editing” refers to an approach for gene editing using napDNAbps, a polymerase (e.g., a reverse transcriptase), and specialized guide RNAs that include a primer binding site and a DNA synthesis template for encoding desired new genetic information (or deleting genetic information) that is then incorporated into a target DNA sequence. Prime editing is described in Anzalone, A. V. et al., Search-and-replace genome editing without double-strand breaks or donor DNA. Nature 576, 149-157 (2019), which is incorporated herein by reference. See also International PCT application, PCT/US2020/023721, filed Mar. 19, 2020, and published as WO 2020/191239, which is incorporated herein by reference.

Prime editing represents a platform for genome editing that is a versatile and precise method to directly write new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“pegRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand (or is homologous to it) immediately downstream of the nick site of the target site to be edited (with the exception that it includes the desired edit). Through DNA repair and/or replication machinery, the endogenous strand downstream of the nick site is replaced by the newly synthesized replacement strand containing the desired edit. In some cases, prime editing may be thought of as a “search-and-replace” genome editing technology since the prime editors, as described herein, not only search and locate the desired target site to be edited, but at the same time, encode a replacement strand containing a desired edit that is installed in place of the corresponding target site endogenous DNA strand. The prime editors of the present disclosure relate, in part, to the discovery that the mechanism of target-primed reverse transcription (TPRT) or “prime editing” can be leveraged or adapted for conducting precision CRISPR/Cas-based genome editing with high efficiency and genetic flexibility. TPRT is naturally used by mobile DNA elements, such as mammalian non-LTR retrotransposons and bacterial Group II introns. Cas protein-reverse transcriptase fusions or related systems are used to target a specific DNA sequence with a guide RNA, generate a single strand nick at the target site, and use the nicked DNA as a primer for reverse transcription of an engineered DNA synthesis template that is integrated with the guide RNA. However, while the concept begins with prime editors that use reverse transcriptase as the DNA polymerase component, the prime editors described herein are not limited to reverse transcriptases but may include the use of virtually any DNA polymerase. Indeed, while the application throughout may refer to prime editors with “reverse transcriptases,” it is set forth here that reverse transcriptases are only one type of DNA polymerase that may work with prime editing. Thus, wherever the specification mentions a “reverse transcriptase,” the person having ordinary skill in the art should appreciate that any suitable DNA polymerase may be used in place of the reverse transcriptase. Thus, in one aspect, the prime editors may comprise Cas9 (or an equivalent napDNAbp), which is programmed to target a DNA sequence by associating it with a specialized guide RNA (i.e., pegRNA) containing a spacer sequence that anneals to a complementary sequence (the complementary sequence to an endogenous protospacer sequence) in the target DNA. The pegRNA also contains new genetic information in the form of an extension that encodes a replacement strand of DNA containing a desired nucleotide change which is used to replace a corresponding endogenous DNA strand at the target site. To transfer information from the pegRNA to the target DNA, the mechanism of prime editing involves nicking the target site in one strand of the DNA to expose a 3′-hydroxyl group. The exposed 3′-hydroxyl group can then be used to prime the DNA polymerization of the edit-encoding extension on pegRNA directly into the target site. In various embodiments, the extension—which provides the template for polymerization of the replacement strand containing the edit—can be formed from RNA or DNA. In the case of an RNA extension, the polymerase of the prime editor can be an RNA-dependent DNA polymerase (such as a reverse transcriptase). In the case of a DNA extension, the polymerase of the prime editor may be a DNA-dependent DNA polymerase. The newly synthesized strand (i.e., the replacement DNA strand containing the desired nucleotide edit) that is formed by the prime editor would be homologous to the genomic target sequence (i.e., have the same sequence as), except for the inclusion of one or more desired nucleotide changes (e.g., a single nucleotide substitution, a deletion, or an insertion, or a combination thereof). The newly synthesized (or replacement) strand of DNA may also be referred to as a single strand DNA flap, which would compete for hybridization with the complementary homologous endogenous DNA strand, thereby displacing the corresponding endogenous strand. Resolution of the hybridized intermediate (also referred to as a heteroduplex, comprising the single strand DNA flap synthesized by the reverse transcriptase hybridized to the endogenous DNA strand with the exception of mismatches at positions where desired nucleotide edits are installed in the edit strand) can include removal of the resulting displaced flap of endogenous DNA (e.g., with a 5′ end DNA flap endonuclease, FEN1), ligation of the synthesized single strand DNA flap to the target DNA, and assimilation of the desired nucleotide changes as a result of cellular DNA repair and/or replication processes.

In various embodiments, prime editing operates by contacting a target DNA molecule (for which a change in the nucleotide sequence is desired to be introduced) with a nucleic acid programmable DNA binding protein (napDNAbp) complexed with a prime editing guide RNA (pegRNA). In various embodiments, the prime editing guide RNA (pegRNA) comprises an extension at the 3′ or 5′ end of the guide RNA, or at an intramolecular location in the guide RNA, and encodes the desired nucleotide change (e.g., single nucleotide substitution, insertion, or deletion). First, the napDNAbp/extended gRNA complex contacts the DNA molecule, and the extended gRNA guides the napDNAbp to bind to a target locus. Next, a nick in one of the strands of DNA of the target locus is introduced (e.g., by a nuclease or chemical agent), thereby creating an available 3′ end in one of the strands of the target locus. In certain embodiments, the nick is created in the strand of DNA that corresponds to the R-loop strand, i.e., the strand that is not hybridized to the guide RNA sequence, i.e., the “non-target strand.” The nick, however, could be introduced in either of the strands. That is, the nick could be introduced into the R-loop “target strand” (i.e., the strand hybridized to the protospacer of the extended gRNA) or the “non-target strand” (i.e., the strand forming the single-stranded portion of the R-loop and which is complementary to the target strand). In the next step, the 3′ end of the DNA strand (formed by the nick) interacts with the extended portion of the guide RNA in order to prime reverse transcription (i.e., “target-primed RT”). In certain embodiments, the 3′ end DNA strand hybridizes to a specific RT priming sequence on the extended portion of the guide RNA, i.e., the “reverse transcriptase priming sequence” or “primer binding site” on the pegRNA. In the next step, a reverse transcriptase (or other suitable DNA polymerase) is introduced that synthesizes a single strand of DNA from the 3′ end of the primed site towards the 5′ end of the prime editing guide RNA. The DNA polymerase (e.g., reverse transcriptase) can be fused to the napDNAbp or alternatively can be provided in trans to the napDNAbp. This forms a single-strand DNA flap comprising the desired nucleotide change (e.g., the single base change, insertion, or deletion, or a combination thereof) and that is otherwise homologous to the endogenous DNA at or adjacent to the nick site. In the next step, the napDNAbp and guide RNA are released. The final two steps relate to the resolution of the single strand DNA flap such that the desired nucleotide change becomes incorporated into the target locus. This process can be driven towards the desired product formation by removing the corresponding 5′ endogenous DNA flap that forms once the 3′ single strand DNA flap invades and hybridizes to the endogenous DNA sequence. Without being bound by theory, the cell's endogenous DNA repair and replication processes resolve the mismatched DNA to incorporate the nucleotide change(s) to form the desired altered product. The process can also be driven towards product formation with “second strand nicking.” This process may introduce at least one or more of the following genetic changes: transversions, transitions, deletions, and insertions.

### Prime Editor

The term “prime editor” refers to the polypeptide or polypeptide components involved in prime editing as described herein. In some embodiments, a prime editor comprises a fusion construct comprising a napDNAbp (e.g., Cas9 nickase, and/or any of the Cas9 variants provided herein) and a reverse transcriptase (e.g., any of the reverse transcriptase variants provided herein). In some embodiments, a prime editor is capable of carrying out prime editing on a target nucleotide sequence in the presence of a pegRNA (or “extended guide RNA”). In some embodiments, a prime editor comprises a napDNAbp (e.g., Cas9 nickase) and a reverse transcriptase provided in trans, i.e., the napDNAbp and the reverse transcriptase are not fused. The in trans napDNAbp and the reverse transcriptase may be tethered via a non-peptide linkage, e.g., an MS2 RNA-protein binding RNA sequence and a MS2 coat protein fused to either the napDNAbp or the reverse transcriptase, or may be unlinked to each other and simply recruited by the pegRNA. In some embodiments, a prime editor composition, system, or complex provided herein comprises a fusion protein or a fusion protein complexed with a pegRNA, and/or further complexed with a second-strand nicking sgRNA. In some embodiments, the prime editor system may also refer to the complex comprising a fusion protein (reverse transcriptase fused to a napDNAbp), a pegRNA, and a regular guide RNA capable of directing the second-site nicking step of the non-edited strand as described herein.

### Protein, Peptide, and Polypeptide

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the contents of which are incorporated herein by reference.

### Protospacer

As used herein, the term “protospacer” refers to the sequence (e.g., of ~20 bp) in DNA adjacent to the PAM (protospacer adjacent motif) sequence. The protospacer shares the same sequence as the spacer sequence of the guide RNA (except that a protospacer contains Thymine and the spacer sequence contains Uracil). The guide RNA anneals to the complement of the protospacer sequence on the target DNA (specifically, one strand thereof, i.e., the “target strand” versus the “non-target strand” of the target DNA sequence). In some embodiments, in order for a Cas nickase component of a prime editor to function, it also requires a specific protospacer adjacent motif (PAM) that varies depending on the Cas protein component itself, e.g., the type of Cas protein and the bacterial species from which it is derived. The most commonly used Cas9 nuclease, derived from S. pyogenes, recognizes a PAM sequence of NGG that is directly downstream of the protospacer sequence in the genomic DNA, on the non-target strand.

### Protospacer Adjacent Motif (PAM)

As used herein, the term “protospacer adjacent motif” or “PAM” refers to a DNA sequence (e.g., an approximately 2-6 nucleotide sequence) that is an important targeting component of a Cas nuclease, e.g., a Cas9. For example, in some embodiments for a Cas9 nuclease, the PAM sequence is on either strand and is downstream in the 5′ to 3′ direction of the Cas9 cut site. The canonical PAM sequence (i.e., the PAM sequence that is associated with the Cas9 nuclease of Streptococcus pyogenes or SpCas9) is 5′-NGG-3′, wherein “N” is any nucleobase followed by two guanine (“G”) nucleobases. In some embodiments, SpCas9 can also recognize additional non-canonical PAMs (e.g., NAG and NGA).

Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes an alternative PAM sequence.

### Reverse Transcriptase

The term “reverse transcriptase” describes a class of polymerases characterized as RNA-dependent DNA polymerases. All known reverse transcriptases require a primer to synthesize a DNA transcript from an RNA template. Historically, reverse transcriptase has been used primarily to transcribe mRNA into cDNA, which can then be cloned into a vector for further manipulation. Avian myoblastosis virus (AMV) reverse transcriptase was the first widely used RNA-dependent DNA polymerase (Verma, Biochim. Biophys. Acta 473:1 (1977)). The enzyme has 5′-3′ RNA-directed DNA polymerase activity, 5′-3′ DNA-directed DNA polymerase activity, and RNase H activity. RNase H is a processive 5′ and 3′ ribonuclease specific for the RNA strand for RNA-DNA hybrids (Perbal, A Practical Guide to Molecular Cloning, New York: Wiley & Sons (1984)). Errors in transcription cannot be corrected by reverse transcriptase because known viral reverse transcriptases lack the 3′-5′ exonuclease activity necessary for proofreading (Saunders and Saunders, Microbial Genetics Applied to Biotechnology, London: Croom Helm (1987)). A detailed study of the activity of AMV reverse transcriptase and its associated RNaseH activity has been presented by Berger et al., Biochemistry 22:2365-2372 (1983). Another reverse transcriptase that is used extensively in molecular biology is reverse transcriptase originating from Moloney murine leukemia virus (M-MLV or “MMLV”). See, e.g., Gerard, G. R., DNA 5:271-279 (1986) and Kotewicz, M. L., et al., Gene 35:249-258 (1985). M-MLV reverse transcriptase substantially lacking in RNase H activity has also been described. See, e.g., U.S. Pat. No. 5,244,797. The invention contemplates the use of any such reverse transcriptases, or variants or mutants thereof.

In some embodiments, the prime editors provided herein comprise MMLV RT, or a variant or fragment of MMLV RT. In some embodiments, the prime editors provided herein comprise Ec48 RT, or a variant or fragment of Ec48 RT. In some embodiments, the prime editors provided herein comprise Tf1 RT, or a variant or fragment of Tf1 RT.

In certain embodiments, a reverse transcriptase comprises the amino acid substitutions E60K, K87E, E165D, D243N, R267I, E279K, K318E, and K343N relative to an Ec48 reverse transcriptase (SEQ ID NO: 7). In certain embodiments, a reverse transcriptase comprises the amino acid substitutions P70T, G72V, S87G, M102I, K106R, K118R, I128V, L158Q, F269L, A363V, K413E, and S492N relative to a Tf1 reverse transcriptase (SEQ ID NO: 1). In certain embodiments, a reverse transcriptase comprises the amino acid substitutions P70T, G72V, S87G, M102I, K106R, K118R, I128V, L158Q, S188K, I260L, F269L, R288Q, S297Q, A363V, K413E, and S492N relative to a Tf1 reverse transcriptase (SEQ ID NO: 1). In certain embodiments, a reverse transcriptase comprises the amino acid substitutions T128N, D200C, and V223Y relative to an MMLV reverse transcriptase (SEQ ID NO: 30) with a truncation of the C-terminal RNaseH domain.

### Reverse Transcription

As used herein, the term “reverse transcription” indicates the capability of an enzyme to synthesize a DNA strand (that is, complementary DNA or cDNA) using RNA as a template. In some embodiments, the reverse transcription can be “error-prone reverse transcription,” which refers to the properties of certain reverse transcriptase enzymes that are error-prone in their DNA polymerization activity.

### Spacer Sequence

As used herein, the term “spacer sequence” in connection with a guide RNA or a pegRNA refers to the portion of the guide RNA or pegRNA of about 20 nucleotides that contains a nucleotide sequence that shares the same sequence as the protospacer sequence in the target DNA sequence. The spacer sequence anneals to the complement of the protospacer sequence to form a ssRNA/ssDNA hybrid structure at the target site and a corresponding R loop ssDNA structure of the endogenous DNA strand.

### Substitution

The term “substitution,” as used herein, refers to replacement of a residue within a sequence, e.g., a nucleic acid or amino acid sequence, with another residue, or a deletion or insertion of one or more residues within a sequence. The term “mutation” may also be used throughout the present disclosure to refer to a substitution (i.e., a “nucleic acid mutation” or an “amino acid mutation”). Substitutions are typically described herein by identifying the original residue followed by the position of the residue within the sequence and the identity of the newly mutated/substituted residue. Various methods for making the amino acid substitutions provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). In some embodiments, a substitution is in a reverse transcriptase, e.g., an MMLV reverse transcriptase, an Ec48 reverse transcriptase, or a Tf1 reverse transcriptase. In some embodiments, a substitution is in a Cas9 protein, e.g., an SpCas9 protein.

### Variant

As used herein, the term “variant” should be taken to mean the exhibition of qualities that have a pattern that deviates from what occurs in nature. The term “variant” encompasses homologous proteins having at least 70%, at least 75%, at least 80%, at least 85%, at least 90%, at least 95%, at least 96%, at least 975, at least 98%, or at least 99% identity with a reference sequence and having the same or substantially the same functional activity or activities as the reference sequence. The term also encompasses mutants, truncations, or domains of a reference sequence that display the same or substantially the same functional activity or activities as the reference sequence.

In some embodiments, a variant comprises one or more, two or more, three or more, four or more, five or more, six or more, seven or more, eight or more, nine or more, or ten or more amino acid substitutions relative to a wild type sequence. In some embodiments, a variant is a reverse transcriptase variant. In certain embodiments, a reverse transcriptase variant comprises one or more, two or more, three or more, four or more, five or more, six or more, seven or more, eight or more, nine or more, or ten or more amino acid substitutions relative to a wild type reverse transcriptase sequence (e.g., wild type MMLV reverse transcriptase, wild type Ec48 reverse transcriptase, or wild type Tf1 reverse transcriptase). In certain embodiments, a Cas9 variant comprises one or more, two or more, three or more, four or more, five or more, six or more, seven or more, eight or more, nine or more, or ten or more amino acid substitutions relative to a wild type Cas9 sequence (e.g., wild type SpCas9) or Cas9 nickase (e.g., SpCas9 nickase).

### Vector

The term “vector,” as used herein, refers to a nucleic acid that can be modified to encode a gene of interest and that is able to enter a host cell, mutate, and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### Wild Type

As used herein the term “wild type” or “WT” is a term of the art understood by skilled persons and means the typical form of an organism, strain, gene, or characteristic as it occurs in nature as distinguished from mutant or variant forms.

### DETAILED DESCRIPTION OF CERTAIN EMBODIMENTS

The present disclosure describes the use of directed evolution and protein engineering to generate new reverse transcriptase and Cas9 variants that enhance editing efficiency when used in the context of prime editors. In particular, next-generation prime editors that are approximately 500-800 bp smaller than PE2 (PE6a and PE6b), while offering mammalian prime editing efficiencies comparable to or higher than those of PE2, were developed. Additionally, highly active and processive prime editors that use either the M-MLV RT or Tf1 RT (PE6c and PE6d) were also developed. These evolved and engineered reverse transcriptases offer substantial improvements over previously used prime editors, e.g., increased editing efficiency for longer edits. Evolved variants of the Cas9 nickase domain of prime editors were also created (PE6e-PE6g), further improving prime editing efficiencies.

Thus, the present disclosure provides evolved and engineered reverse transcriptase variants and Cas9 variants with improved properties (e.g., improved editing efficiency when used in the context of a prime editor). Fusion proteins, including for example prime editors, comprising the reverse transcriptase variants and Cas9 variants described herein are also provided by the present disclosure. The present disclosure also provides polynucleotides encoding the reverse transcriptase variants, Cas9 variants, fusion proteins, and prime editors provided herein, as well as vectors comprising such polynucleotides. Pharmaceutical compositions, AAVs and cells comprising the reverse transcriptase variants, Cas9 variants, and prime editors (and/or polynucleotides or vectors encoding the same) described herein are also provided by the present disclosure. The present disclosure also provides methods and uses involving the reverse transcriptase variants, Cas9 variants, and prime editors described herein.

### PE6 Prime Editors, Cas9 Variants, Reverse Transcriptase Variants, and Fusion Proteins

Some aspects of the present disclosure provide evolved and/or engineered reverse transcriptases and Cas9 proteins, and prime editors comprising the same, with various improved properties (e.g., smaller size to increase delivery efficiency (for example, using AAVs), improved prime editing efficiency (for example, for edits that require structured pegRNA RT templates), decreased frequency of indels, etc.). In some embodiments, pegRNA structure and folding prediction, including free energy of the folding of pegRNA components, e.g., the RT template or the extension arm, can be measured by NUPACK free energy prediction as described in Zadeh, J. N. et al., (2011). NUPACK: Analysis and design of nucleic acid systems. J. Comput. Chem. 32, 170-173, which is incorporated herein by reference.

The variants provided by the present disclosure include variants of Escherichia coli Ec48 reverse transcriptase, Schizosaccharomyces pombe Tf1 reverse transcriptase, Moloney murine leukemia virus (MMLV) reverse transcriptase, and Streptococcus pyogenes Cas9, as well as variants comprising the amino acid substitutions disclosed herein at corresponding positions in homologous proteins.

In one aspect, the present disclosure provides reverse transcriptase variants comprising various amino acid substitutions relative to the amino acid sequence of Schizosaccharomyces pombe Tf1 reverse transcriptase, which is provided below:

(SEQ ID NO: 1)
ISSSKHTLSQMNKVSNIVKEPELPDIYKEFKDITADTNTEKLPKPIKGLEFEVELTQEN
YRLPIRNYPLPPGKMQAMNDEINQGLKSGIIRESKAINACPVMFVPKKEGTLRMVVD
YKPLNKYVKPNIYPLPLIEQLLAKIQGSTIFTKLDLKSAYHLIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFIGYHISEKGFTPCQENIDKVLQWKQPKNRKE
LRQFLGSVNYLRKFIPKTSQLTHPLNKLLKKDVRWKWTPTQTQAIENIKQCLVSPPV
LRHFDFSKKILLETDASDVAVGAVLSQKHDDDKYYPVGYYSAKMSKAQLNYSVSD
KEMLAIIKSLKHWRHYLESTIEPFKILTDHRNLIGRITNESEPENKRLARWQLFLQDEN
FEINYRPGSANHIADALSRIVDETEPIPKDSEDNSINFVNQISI

In some embodiments, the present disclosure provides reverse transcriptase variants having at least 80%, at least 85%, at least 90%, at least 95%, at least 96%, at least 97%, at least 98%, or at least 99% sequence identity with SEQ ID NO: 1, wherein the reverse transcriptase variant comprises amino acid substitutions at positions 70, 72, 87, 102, 106, 118, 128, 158, 269, 363, 413, and 492 relative to SEQ ID NO: 1, or corresponding substitutions in a homologous sequence. In some embodiments, the amino acid substitution at position 70 is a P70X substitution, wherein X is any amino acid other than wild type. In certain embodiments, the amino acid substitution at position 70 is a P70T substitution. In some embodiments, the amino acid substitution at position 72 is a G72X substitution, wherein X is any amino acid other than wild type. In certain embodiments, the amino acid substitution at position 72 is a G72V substitution. In some embodiments, the amino acid substitution at position 87 is an S87X substitution, wherein X is any amino acid other than wild type. In certain embodiments, the amino acid substitution at position 87 is an S87G substitution. In some embodiments, the amino acid substitution at position 102 is an M102X substitution, wherein X is any amino acid other than wild type. In certain embodiments, the amino acid substitution at position 102 is an M102I substitution. In some embodiments, the amino acid substitution at position 106 is a K106X substitution, wherein X is any amino acid other than wild type. In certain embodiments, the amino acid substitution at position 106 is a K106R substitution. In some embodiments, the amino acid substitution at position 118 is a K118X substitution, wherein X is any amino acid other than wild type. In certain embodiments, the amino acid substitution at position 118 is a K118R substitution. In some embodiments, the amino acid substitution at position 128 is an I128X substitution, wherein X is any amino acid other than wild type. In certain embodiments, the amino acid substitution at position 128 is an I128V substitution. In some embodiments, the amino acid substitution at position 158 is an L158X substitution, wherein X is any amino acid other than wild type. In certain embodiments, the amino acid substitution at position 158 is an L158Q substitution. In some embodiments, the amino acid substitution at position 269 is an F269X substitution, wherein X is any amino acid other than wild type. In certain embodiments, the amino acid substitution at position 269 is an F269L substitution. In some embodiments, the amino acid substitution at position 363 is an A363X substitution, wherein X is any amino acid other than wild type. In certain embodiments, the amino acid substitution at position 363 is an A363V substitution. In some embodiments, the amino acid substitution at position 413 is a K413X substitution, wherein X is any amino acid other than wild type. In certain embodiments, the amino acid substitution at position 413 is a K413E substitution. In some embodiments, the amino acid substitution at position 492 is an S492X substitution, wherein X is any amino acid other than wild type. In certain embodiments, the amino acid substitution at position 492 is an S492N substitution. In certain embodiments, the reverse transcriptase variant comprises the substitutions P70T, G72V, S87G, M102I, K106R, K118R, I128V, L158Q, F269L, A363V, K413E, and S492N relative to SEQ ID NO: 1. In some embodiments, the reverse transcriptase variants further comprise amino acid substitutions at positions 188, 260, 297, and 288 relative to SEQ ID NO: 1. In some embodiments, the amino acid substitution at position 188 is an S188X substitution, wherein X is any amino acid other than wild type. In certain embodiments, the amino acid substitution at position 188 is an S188K substitution. In some embodiments, the amino acid substitution at position 260 is an I260X substitution, wherein X is any amino acid other than wild type. In certain embodiments, the amino acid substitution at position 260 is an I260L substitution. In some embodiments, the amino acid substitution at position 297 is an S297X substitution, wherein X is any amino acid other than wild type. In certain embodiments, the amino acid substitution at position 297 is an S297Q substitution. In some embodiments, the amino acid substitution at position 288 is an R288X substitution, wherein X is any amino acid other than wild type. In certain embodiments, the amino acid substitution at position 288 is an R288Q substitution. In certain embodiments, the reverse transcriptase variant further comprises the substitutions S188K, 1260L, S297Q, and R288Q relative to SEQ ID NO: 1.

In another aspect, the present disclosure provides reverse transcriptase variants comprising various amino acid substitutions relative to the amino acid sequence of MMLV reverse transcriptase, which is provided below:

(SEQ ID NO: 30)
TLNIEDEYRLHETSKEPDVSLGSTWLSDFPQAWAETGGMGLAVRQAPLIIPLKATSTP
VSIKQYPMSQEARLGIKPHIQRLLDQGILVPCQSPWNTPLLPVKKPGTNDYRPVQDLR
EVNKRVEDIHPTVPNPYNLLSGLPPSHQWYTVLDLKDAFFCLRLHPTSQPLFAFEWR
DPEMGISGQLTWTRLPQGFKNSPTLFDEALHRDLADFRIQHPDLILLQYVDDLLLAAT
SELDCQQGTRALLQTLGNLGYRASAKKAQICQKQVKYLGYLLKEGQRWLTEARKE
TVMGQPTPKTPRQLREFLGTAGFCRLWIPGFAEMAAPLYPLTKTGTLFNWGPDQQK
AYQEIKQALLTAPALGLPDLTKPFELFVDEKQGYAKGVLTQKLGPWRRPVAYLSKK
LDPVAAGWPPCLRMVAAIAVLTKDAGKLTMGQPLVILAPHAVEALVKQPPDRWLS
NARMTHYQALLLDTDRVQFGPVVALNPATLLPLPEEGLQHNCLDILAEAHGTRPDL
TDQPLPDADHTWYTDGSSLLQEGQRKAGAAVTTETEVIWAKALPAGTSAQRAELIA
LTQALKMAEGKKLNVYTDSRYAFATAHIHGEIYRRRGLLTSEGKEIKNKDEILALLK
ALFLPKRLSIIHCPGHQKGHSAEARGNRMADQAARKAAITETPDTSTLLIENSSP.

In some embodiments, the present disclosure provides reverse transcriptase variants having at least 80%, at least 85%, at least 90%, at least 95%, at least 96%, at least 97%, at least 98%, or at least 99% sequence identity with SEQ ID NO: 30, wherein the reverse transcriptase variant comprises amino acid substitutions at positions 128 and 200 relative to SEQ ID NO: 30, or corresponding substitutions in a homologous sequence. In some embodiments, the substitution at position 128 is a T128X substitution, wherein X is any amino acid other than wild type. In certain embodiments, the substitution at position 128 is a T128N substitution. In some embodiments, the substitution at position 200 is a D200X substitution, wherein X is any amino acid other than wild type. In certain embodiments, the substitution at position 200 is a D200C substitution. In certain embodiments, the reverse transcriptase variant comprises the amino acid substitutions T128N and D200C. In some embodiments, the reverse transcriptase variant further comprises an amino acid substitution at position 223 relative to SEQ ID NO: 30. In some embodiments, the amino acid substitution at position 223 is a V223X substitution, wherein X is any amino acid other than wild type. In certain embodiments, the amino acid substitution at position 223 is a V223Y substitution. In some embodiments, the reverse transcriptase variant further comprises amino acid substitutions from the MMLV reverse transcriptase used in PE2 and PEmax (e.g., the amino acid substitutions T306K, W313F, and T330P). In certain embodiments, the reverse transcriptase variant comprises a truncation of all or part of the C-terminal RNaseH domain of MMLV reverse transcriptase (e.g., a truncation at amino acid position 490, 491, 492, 493, 494, 495, 496, 497, 498, 499, 500, 501, 502, 503, 504, 505, 506, 507, 508, 509, or 510 of SEQ ID NO: 30). In certain embodiments, the reverse transcriptase variant comprises a truncation between positions D497 and I498 of SEQ ID NO: 30.

In some embodiments, the present disclosure provides reverse transcriptase variants having at least 80%, at least 85%, at least 90%, at least 95%, at least 96%, at least 97%, at least 98%, or at least 99% sequence identity with SEQ ID NO: 30, wherein the reverse transcriptase variant comprises the amino acid substitutions T128N and V223M relative to SEQ ID NO: 30, or corresponding substitutions in a homologous sequence. In some embodiments, the present disclosure provides reverse transcriptase variants having at least 80%, at least 85%, at least 90%, at least 95%, at least 96%, at least 97%, at least 98%, or at least 99% sequence identity with SEQ ID NO: 30, wherein the reverse transcriptase variant comprises the amino acid substitutions T128N and V223Y relative to SEQ ID NO: 30, or corresponding substitutions in a homologous sequence. In some embodiments, the present disclosure provides reverse transcriptase variants having at least 80%, at least 85%, at least 90%, at least 95%, at least 96%, at least 97%, at least 98%, or at least 99% sequence identity with SEQ ID NO: 30, wherein the reverse transcriptase variant comprises the amino acid substitutions T128F and V223M relative to SEQ ID NO: 30, or corresponding substitutions in a homologous sequence. In some embodiments, the present disclosure provides reverse transcriptase variants having at least 80%, at least 85%, at least 90%, at least 95%, at least 96%, at least 97%, at least 98%, or at least 99% sequence identity with SEQ ID NO: 30, wherein the reverse transcriptase variant comprises the amino acid substitutions D200C and V223M relative to SEQ ID NO: 30, or corresponding substitutions in a homologous sequence.

In some embodiments, the present disclosure provides reverse transcriptase variants having at least 80%, at least 85%, at least 90%, at least 95%, at least 96%, at least 97%, at least 98%, or at least 99% sequence identity with SEQ ID NO: 30, wherein the reverse transcriptase variant comprises amino acid substitutions at positions 128, 129, 196, 200, and 223 relative to SEQ ID NO: 30, or corresponding substitutions in a homologous sequence. In some embodiments, the amino acid substitution at position 128 is a T128X substitution, wherein X is any amino acid other than wild type. In certain embodiments, the amino acid substitution at position 128 is a T128N substitution. In some embodiments, the amino acid substitution at position 129 is a V129X substitution, wherein X is any amino acid other than wild type. In certain embodiments, the amino acid substitution at position 129 is a V129A substitution. In certain embodiments, the amino acid substitution at position 129 is a V129G substitution. In some embodiments, the amino acid substitution at position 196 is a P196X substitution, wherein X is any amino acid other than wild type. In certain embodiments, the amino acid substitution at position 196 is a P196S substitution. In certain embodiments, the amino acid substitution at position 196 is a P196T substitution. In certain embodiments, the amino acid substitution at position 196 is a P196F substitution. In some embodiments, the amino acid substitution at position 200 is an N200X substitution, wherein X is any amino acid other than wild type. In certain embodiments, the amino acid substitution at position 200 is an N200S substitution. In certain embodiments, the amino acid substitution at position 200 is an N200Y substitution. In some embodiments, the amino acid substitution at position 223 is a V223X substitution, wherein X is any amino acid other than wild type. In certain embodiments, the amino acid substitution at position 223 is a V223A substitution. In certain embodiments, the amino acid substitution at position 223 is a V223M substitution. In certain embodiments, the amino acid substitution at position 223 is a V223L substitution. In certain embodiments, the amino acid substitution at position 223 is a V223E substitution.

In some embodiments, any of the reverse transcriptase variants provided herein further comprise amino acid substitutions from the MMLV reverse transcriptase used in PE2 and PEmax (e.g., any of the amino acid substitutions D200N, T306K, W313F, T330P, and L603W). In certain embodiments, any of the reverse transcriptase variants provided herein comprise a truncation of all or part of the C-terminal RNaseH domain of MMLV reverse transcriptase (e.g., a truncation at amino acid position 490, 491, 492, 493, 494, 495, 496, 497, 498, 499, 500, 501, 502, 503, 504, 505, 506, 507, 508, 509, or 510 of SEQ ID NO: 30). In certain embodiments, any of the reverse transcriptase variants provided herein comprise a truncation between positions D497 and I498 of SEQ ID NO: 30.

In some embodiments, the reverse transcriptase variant comprises the amino acid sequence of SEQ ID NO: 25 (the RT domain of “PE6b”), or an amino acid sequence at least 80%, at least 85%, at least 90%, at least 95%, at least 96%, at least 97%, at least 98%, or at least 99% identical to the amino acid sequence of SEQ ID NO: 25:

(SEQ ID NO: 25)
ISSSKHTLSQMNKVSNIVKEPELPDIYKEFKDITADTNTEKLPKPIKGLEFEVELTQEN
YRLPIRNYPLTPVKMQAMNDEINQGLKGGIIRESKAINACPVIFVPRKEGTLRMVVDY
RPLNKYVKPNVYPLPLIEQLLAKIQGSTIFTKLDLKSAYHQIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFIGYHISEKGLTPCQENIDKVLQWKQPKNRKE
LRQFLGSVNYLRKFIPKTSQLTHPLNKLLKKDVRWKWTPTQTQAIENIKQCLVSPPV
LRHFDFSKKILLETDVSDVAVGAVLSQKHDDDKYYPVGYYSAKMSKAQLNYSVSD
KEMLAIIKSLEHWRHYLESTIEPFKILTDHRNLIGRITNESEPENKRLARWQLFLQDEN
FEINYRPGSANHIADALSRIVDETEPIPKDNEDNSINFVNQISI

In some embodiments, a PE6b prime editor comprises the amino acid sequence:

(SEQ ID NOs: 146, 147)
MKRTADGSEFESPKKKRKV[CAS9]SGGSSGGSKRTADGSEFESPKKKRKVSGGSSGG
SISSSKHTLSQMNKVSNIVKEPELPDIYKEFKDITADTNTEKLPKPIKGLEFEVELTQEN
YRLPIRNYPLTPVKMQAMNDEINQGLKGGIIRESKAINACPVIFVPRKEGTLRMVVDY
RPLNKYVKPNVYPLPLIEQLLAKIQGSTIFTKLDLKSAYHQIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFIGYHISEKGLTPCQENIDKVLQWKQPKNRKE
LRQFLGSVNYLRKFIPKTSQLTHPLNKLLKKDVRWKWTPTQTQAIENIKQCLVSPPV
LRHFDFSKKILLETDVSDVAVGAVLSQKHDDDKYYPVGYYSAKMSKAQLNYSVSD
KEMLAIIKSLEHWRHYLESTIEPFKILTDHRNLIGRITNESEPENKRLARWQLFLQDFN
FEINYRPGSANHIADALSRIVDETEPIPKDNEDNSINFVNQISIKRTADGSEFESPKKKR
KVPAAKRVKLD,
wherein [CAS9] comprises any Cas9 protein
(e.g., any of the Cas9 variants disclosed herein).

In some embodiments, the reverse transcriptase variant comprises the amino acid sequence of SEQ ID NO: 26 (the RT domain of “PE6c”), or an amino acid sequence at least 80%, at least 85%, at least 90%, at least 95%, at least 96%, at least 97%, at least 98%, or at least 99% identical to the amino acid sequence of SEQ ID NO: 26:

(SEQ ID NO: 26)
ISSSKHTLSQMNKVSNIVKEPELPDIYKEFKDITADTNTEKLPKPIKGLEFEVELTQEN
YRLPIRNYPLTPVKMQAMNDEINQGLKGGIIRESKAINACPVIFVPRKEGTLRMVVDY
RPLNKYVKPNVYPLPLIEQLLAKIQGSTIFTKLDLKSAYHQIRVRKGDEHKLAFRCPR
GVFEYLVMPYGIKTAPAHFQYFINTILGEAKESHVVCYMDDILIHSKSESEHVKHVK
DVLQKLKNANLIINQAKCEFHQSQVKFLGYHISEKGLTPCQENIDKVLQWKQPKNQ
KELRQFLGQVNYLRKFIPKTSQLTHPLNKLLKKDVRWKWTPTQTQAIENIKQCLVSP
PVLRHFDFSKKILLETDVSDVAVGAVLSQKHDDDKYYPVGYYSAKMSKAQLNYSVS
DKEMLAIIKSLEHWRHYLESTIEPFKILTDHRNLIGRITNESEPENKRLARWQLFLQDF
NFEINYRPGSANHIADALSRIVDETEPIPKDNEDNSINFVNQISI.

In some embodiments, a PE6c prime editor comprises the amino acid sequence:

(SEQ ID NOs: 146, 148)
MKRTADGSEFESPKKKRKV[CAS9]SGGSSGGSKRTADGSEFESPKKKRKVSGGSSGG
SISSSKHTLSQMNKVSNIVKEPELPDIYKEFKDITADTNTEKLPKPIKGLEFEVELTQEN
YRLPIRNYPLTPVKMQAMNDEINQGLKGGIIRESKAINACPVIFVPRKEGTLRMVVDY
RPLNKYVKPNVYPLPLIEQLLAKIQGSTIFTKLDLKSAYHQIRVRKGDEHKLAFRCPR
GVFEYLVMPYGIKTAPAHFQYFINTILGEAKESHVVCYMDDILIHSKSESEHVKHVK
DVLQKLKNANLIINQAKCEFHQSQVKFLGYHISEKGLTPCQENIDKVLQWKQPKNQ
KELRQFLGQVNYLRKFIPKTSQLTHPLNKLLKKDVRWKWTPTQTQAIENIKQCLVSP
PVLRHFDFSKKILLETDVSDVAVGAVLSQKHDDDKYYPVGYYSAKMSKAQLNYSVS
DKEMLAIIKSLEHWRHYLESTIEPFKILTDHRNLIGRITNESEPENKRLARWQLFLQDF
NFEINYRPGSANHIADALSRIVDETEPIPKDNEDNSINFVNQISIKRTADGSEFESPKKK
RKVPAAKRVKLD,
wherein [CAS9] comprises any Cas9 protein
(e.g., any of the Cas9 variants disclosed herein).

In some embodiments, the reverse transcriptase variant comprises the amino acid sequence of SEQ ID NO: 27 (the RT domain of “PE6d”), or an amino acid sequence at least 80%, at least 85%, at least 90%, at least 95%, at least 96%, at least 97%, at least 98%, or at least 99% identical to the amino acid sequence of SEQ ID NO: 27:

(SEQ ID NO: 27)
TLNIEDEYRLHETSKEPDVSLGSTWLSDFPQAWAETGGMGLAVRQAPLIIPLKATSTP
VSIKQYPMSQEARLGIKPHIQRLLDQGILVPCQSPWNTPLLPVKKPGTNDYRPVQDLR
EVNKRVEDIHPNVPNPYNLLSGLPPSHQWYTVLDLKDAFFCLRLHPTSQPLFAFEWR
DPEMGISGQLTWTRLPQGFKNSPTLFCEALHRDLADFRIQHPDLILLQYYDDLLLAAT
SELDCQQGTRALLQTLGNLGYRASAKKAQICQKQVKYLGYLLKEGQRWLTEARKE
TVMGQPTPKTPRQLREFLGKAGFCRLFIPGFAEMAAPLYPLTKPGTLFNWGPDQQKA
YQEIKQALLTAPALGLPDLTKPFELFVDEKQGYAKGVLTQKLGPWRRPVAYLSKKL
DPVAAGWPPCLRMVAAIAVLTKDAGKLTMGQPLVILAPHAVEALVKQPPDRWLSN
ARMTHYQALLLDTDRVQFGPVVALNPATLLPLPEEGLQHNCLD.

In some embodiments, a PE6d prime editor comprises the amino acid sequence: [optional NLS]-[CAS9]-[SEQ ID NO:27]-[optional NLS]

wherein CAS9 comprises any Cas9 protein (e.g., any of the Cas9 variants disclosed herein), and wherein “optional NLS” comprises one or more nuclear localization signals described herein or known in the art, and wherein each of the “]-[” independently comprises a optional peptide linker described herein or known in the art.

In some embodiments, the N-terminal NLS of the PE6d prime editor comprises a bipartite SV40 NLS as set forth in SEQ ID NO: 95.

In some embodiments, the C-terminal NLS of the PE6d prime editor comprises a bipartite SV40 NLS as set forth in SEQ ID NO: 97. In some embodiments, the C-myc NLS of the PE6d prime editor comprises a bipartite SV40 NLS as set forth in SEQ ID NO: 98. In some embodiments, the C-terminal NLS of the PE6d prime editor comprises the sequence

SGGSKRTADGSEFESPKKKRKVGSGPAAKRVKLD.

In some embodiments, the C-terminal NLS of the PE6d prime editor comprises a bipartite SV40 NLS as set forth in SEQ ID NO: 96.

In some embodiments, the peptide linker connecting the Cas9 and the MMLV-RT variant of a PE6d fusion protein comprises SEQ ID NO: 80.

In some embodiments, the peptide linker connecting the Cas9 and the MMLV-RT variant of a PE6d fusion protein comprises SEQ ID NO: 79.

In some embodiments, a PE6d prime editor comprises the amino acid sequence:

(SEQ ID NOs: 146, 149)
MKRTADGSEFESPKKKRKV[CAS9]SGGSSGGSKRTADGSEFESPKKKRKVSGGSSGG
STLNIEDEYRLHETSKEPDVSLGSTWLSDFPQAWAETGGMGLAVRQAPLIIPLKATST
PVSIKQYPMSQEARLGIKPHIQRLLDQGILVPCQSPWNTPLLPVKKPGTNDYRPVQDL
REVNKRVEDIHPNVPNPYNLLSGLPPSHQWYTVLDLKDAFFCLRLHPTSQPLFAFEW
RDPEMGISGQLTWTRLPQGFKNSPTLFCEALHRDLADFRIQHPDLILLQYYDDLLLAA
TSELDCQQGTRALLQTLGNLGYRASAKKAQICQKQVKYLGYLLKEGQRWLTEARK
ETVMGQPTPKTPRQLREFLGKAGFCRLFIPGFAEMAAPLYPLTKPGTLFNWGPDQQK
AYQEIKQALLTAPALGLPDLTKPFELFVDEKQGYAKGVLTQKLGPWRRPVAYLSKK
LDPVAAGWPPCLRMVAAIAVLTKDAGKLTMGQPLVILAPHAVEALVKQPPDRWLS
NARMTHYQALLLDTDRVQFGPVVALNPATLLPLPEEGLQHNCLDKRTADGSEFESP
KKKRKVPAAKRVKLD,
wherein [CAS9] comprises any Cas9
protein (e.g., any of the Cas9 variants disclosed herein).

In another aspect, the present disclosure provides Cas9 variants comprising various amino acid substitutions relative to the amino acid sequence of Streptococcus pyogenes Cas9 nickase (H840A), which is provided below:

(SEQ ID NO: 2)
MDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENT
QLQNEKLYLYYLQNGRDMYVDQELDINRLSDYDVDAIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGD.

In some embodiments, the Cas9 comprises the amino acid sequence as set forth in SEQ ID NO: 10.

In some embodiments, the Cas9 comprises the amino acid sequence as set forth in SEQ ID NO: 11.

In some embodiments, the Cas9 comprises the amino acid sequence as set forth in SEQ ID NO: 133.

In some embodiments, the present disclosure provides Cas9 variants having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 775 and 918 relative to SEQ ID NO: 2, or corresponding substitutions in a homologous sequence. In some embodiments, the amino acid substitution at position 775 is a K775X substitution, wherein X is any amino acid other than wild type. In certain embodiments, the amino acid substitution at position 775 is a K775R substitution. In some embodiments, the amino acid substitution at position 918 is a K918X substitution, wherein X is any amino acid other than wild type. In certain embodiments, the amino acid substitution at position 918 is a K918A substitution. In certain embodiments, the Cas9 variant comprises K775R and K918A substitutions relative to SEQ ID NO: 2.

In some embodiments, the present disclosure provides Cas9 variants having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 99, 471, 632, 645, and 721 relative to SEQ ID NO: 2, or corresponding substitutions in a homologous sequence. In some embodiments, the amino acid substitution at position 99 is an H99X substitution, wherein X is any amino acid. In certain embodiments, the amino acid substitution at position 99 is an H99R substitution. In some embodiments, the amino acid substitution at position 471 is an E471X substitution, wherein X is any amino acid. In certain embodiments, the amino acid substitution at position 471 is an E471K substitution. In some embodiments, the amino acid substitution at position 632 is an I632X substitution, wherein X is any amino acid. In certain embodiments, the amino acid substitution at position 632 is an I632V substitution. In some embodiments, the amino acid substitution at position 645 is a D645X substitution, wherein X is any amino acid. In certain embodiments, the amino acid substitution at position 645 is a D645N substitution. In some embodiments, the amino acid substitution at position 721 is an H721X substitution, wherein X is any amino acid. In certain embodiments, the amino acid substitution at position 721 is an H721Y substitution. In some embodiments, the Cas9 variant further comprises an amino acid substitution at position 654 relative to SEQ ID NO: 2. In some embodiments, the amino acid substitution at position 654 is an R654X substitution, wherein X is any amino acid other than wild type. In certain embodiments, the amino acid substitution at position 654 is an R654C substitution. In some embodiments, the Cas9 variant further comprises an amino acid substitution at position 918 relative to SEQ ID NO: 2. In some embodiments, the amino acid substitution at position 918 is a K918X substitution, wherein X is any amino acid other than wild type. In certain embodiments, the amino acid substitution at position 918 is a K918A substitution.

In some embodiments, the present disclosure provides Cas9 variants having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 99, 471, and 632 relative to SEQ ID NO: 2, or corresponding substitutions in a homologous sequence. In some embodiments, the amino acid substitution at position 99 is an H99X substitution, wherein X is any amino acid other than wild type. In certain embodiments, the amino acid substitution at position 99 is an H99R substitution. In some embodiments, the amino acid substitution at position 471 is an E471X substitution, wherein X is any amino acid other than wild type. In certain embodiments, the amino acid substitution at position 471 is an E471K substitution. In some embodiments, the amino acid substitution at position 632 is an I632X substitution, wherein X is any amino acid other than wild type. In certain embodiments, the amino acid substitution at position 632 is an I632V substitution. In certain embodiments, the Cas9 variant comprises the amino acid substitutions H99R, E471K, and I632V relative to SEQ ID NO: 2. In some embodiments, the Cas9 variant further comprises an amino acid substitution at position 721 relative to SEQ ID NO: 2. In some embodiments, the amino acid substitution at position 721 is an H721X substitution, wherein X is any amino acid other than wild type. In certain embodiments, the amino acid substitution at position 721 is an H721K substitution.

In some embodiments, the present disclosure provides Cas9 variants having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 471 and 918 relative to SEQ ID NO: 2, or corresponding substitutions in a homologous sequence. In some embodiments, the amino acid substitution at position 471 is an E471X substitution, wherein X is any amino acid other than wild type. In certain embodiments, the amino acid substitution at position 471 is an E471K substitution. In some embodiments, the amino acid substitution at position 918 is a K918X substitution, wherein X is any amino acid other than wild type. In certain embodiments, the amino acid substitution at position 918 is a K918A substitution. In certain embodiments, the Cas9 variant comprises the amino acid substitutions E471K and K918A relative to SEQ ID NO: 2.

In some embodiments, the present disclosure provides Cas9 variants having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 753 and 1151 relative to SEQ ID NO: 2, or corresponding substitutions in a homologous sequence. In some embodiments, the amino acid substitution at position 753 is an R753X substitution, wherein X is any amino acid other than wild type. In certain embodiments, the amino acid substitution at position 753 is an R753G substitution. In some embodiments, the amino acid substitution at position 1151 is a K1151X substitution, wherein X is any amino acid other than wild type. In certain embodiments, the amino acid substitution at position 1151 is a K1151E substitution. In certain embodiments, the Cas9 variant comprises the amino acid substitutions R753G and K1151E relative to SEQ ID NO: 2.

In some embodiments, the present disclosure provides Cas9 variants having at least 80%, at least 85%, at least 90%, at least 95%, at least 96%, at least 97%, at least 98%, or at least 99% sequence identity with SEQ ID NO: 2, wherein the Cas9 variant comprises one or more, two or more, three or more, four or more, five or more, six or more, seven or more, eight or more, nine or more, or ten amino acid substitutions at positions selected from the group consisting of 260, 298, 395, 769, 778, 1014, 1034, 1100, 1106, 1138, 1152, and 1320 relative to SEQ ID NO: 2, or corresponding substitutions in a homologous sequence. In some embodiments, the amino acid substitution at position 260 is an E260X substitution, wherein X is any amino acid other than wild type. In certain embodiments, the amino acid substitution at position 260 is an E260K substitution. In some embodiments, the amino acid substitution at position 298 is a D298X substitution, wherein X is any amino acid other than wild type. In certain embodiments, the amino acid substitution at position 298 is a D298N substitution. In some embodiments, the amino acid substitution at position 395 is an R395X substitution, wherein X is any amino acid other than wild type. In certain embodiments, the amino acid substitution at position 395 is an R395C substitution. In some embodiments, the amino acid substitution at position 769 is a T769X substitution, wherein X is any amino acid other than wild type. In certain embodiments, the amino acid substitution at position 769 is a T769P substitution. In some embodiments, the amino acid substitution at position 778 is an R778X substitution, wherein X is any amino acid other than wild type. In certain embodiments, the amino acid substitution at position 778 is an R778Q substitution. In some embodiments, the amino acid substitution at position 1014 is a K1014X substitution, wherein X is any amino acid other than wild type. In certain embodiments, the amino acid substitution at position 1014 is a K1014E substitution. In some embodiments, the amino acid substitution at position 1034 is an A1034X substitution, wherein X is any amino acid other than wild type. In certain embodiments, the amino acid substitution at position 1034 is an A1034E substitution. In some embodiments, the amino acid substitution at position 1100 is a V1100X substitution, wherein X is any amino acid other than wild type. In certain embodiments, the amino acid substitution at position 1100 is a V1100I substitution. In some embodiments, the amino acid substitution at position 1106 is an S1106X substitution, wherein X is any amino acid other than wild type. In certain embodiments, the amino acid substitution at position 1106 is an S1106F substitution. In some embodiments, the amino acid substitution at position 1138 is a T1138X substitution, wherein X is any amino acid other than wild type. In certain embodiments, the amino acid substitution at position 1138 is a T1138A substitution. In some embodiments, the amino acid substitution at position 1152 is a G1152X substitution, wherein X is any amino acid other than wild type. In certain embodiments, the amino acid substitution at position 1152 is a G1152E substitution. In some embodiments, the amino acid substitution at position 1320 is an A1320X substitution, wherein X is any amino acid other than wild type. In certain embodiments, the amino acid substitution at position 1320 is an A1320T substitution. In certain embodiments, the Cas9 variant comprises one or more, two or more, three or more, four or more, five or more, six or more, seven or more, eight or more, nine or more, or ten or more amino acid substitutions E260K, D298N, R395C, T769P, R778Q, K1014E, A1034E, V1100I, S1106F, T1138A, G1152E, and A1320T. In certain embodiments, the Cas9 variant comprises the amino acid substitutions E260K, D298N, R395C, T769P, R778Q, K1014E, A1034E, V1100I, S1106F, T1138A, G1152E, and A1320T. In some embodiments, the Cas9 variant further comprises one or more additional amino acid substitutions at positions selected from the group consisting of 102, 753, 804, and 1003 relative to SEQ ID NO: 2. In some embodiments, the amino acid substitution at position 102 is an E102X substitution, wherein X is any amino acid other than wild type. In certain embodiments, the amino acid substitution at position 102 is an E102K substitution. In some embodiments, the amino acid substitution at position 753 is an R753X substitution, wherein X is any amino acid other than wild type. In certain embodiments, the amino acid substitution at position 753 is an R753G substitution. In some embodiments, the amino acid substitution at position 804 is a T804X substitution, wherein X is any amino acid other than wild type. In certain embodiments, the amino acid substitution at position 804 is a T804A substitution. In some embodiments, the amino acid substitution at position 1003 is a K1003X substitution, wherein X is any amino acid other than wild type. In certain embodiments, the amino acid substitution at position 1003 is a K1003R substitution. In some embodiments, the Cas9 variant comprises amino acid substitutions at positions 102, 395, 753, 778, and 1100; 753, 769, 1034, and 1320; 298, 753, 1034, and 1138; 102, 260, 395, 753, 778, 804, 1003, 1100, 1106, and 1152; or 102, 260, 395, 753, 778, 804, 1003, 1014, 1100, 1106, and 1152; relative to SEQ ID NO: 2. In certain embodiments, the Cas9 variant comprises amino acid substitutions at the positions: E102K, R395C, R753G, R778Q, and V1100I; R753G, T769P, A1034E, and A1320T; D298N, R753G, A1034E, and T1138A; E102K, E260K, R395C, R753C, R778Q, T804A, K1003R, V1100I, S1106F, and G1152E; or E102K, E260K, R395C, R753G, R778Q, T804A, K1003R, K1014E, V1100I, S1106F, and G1152E; relative to SEQ ID NO: 2.

In some embodiments, the present disclosure provides Cas9 variants having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 23 and 754 relative to SEQ ID NO: 2, or corresponding substitutions in a homologous sequence. In some embodiments, the amino acid substitution at position 23 is a D23X substitution, wherein X is any amino acid other than wild type (i.e., D).

In certain embodiments, the amino acid substitution at position 23 is a D23G substitution. In some embodiments, the amino acid substitution at position 754 is an H754X substitution, wherein X is any amino acid other than wild type (i.e., H). In certain embodiments, the amino acid substitution at position 754 is an H754R substitution.

In certain embodiments, the Cas9 variant comprises the amino acid sequence of SEQ ID NO: 28 (the Cas9 domain of “PE6e”), or an amino acid sequence at least 80%, at least 85%, at least 90%, at least 95%, at least 96%, at least 97%, at least 98%, or at least 99% identical to the amino acid sequence of SEQ ID NO: 28:

(SEQ ID NO: 28)
MDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDEL
VKVMGRHKPENIVIEMARENQTTQKGQRNSRERMKRIEEGIKELGSQILKEHPVENT
QLQNEKLYLYYLQNGRDMYVDQELDINRLSDYDVDAIVPQSFLKDDSIDNKVLTRS
DKNRGKSDNVPSEEVVKKMKNYWRQLLNAKLITQRKFDNLTKAERGGLSELDKAG
FIARQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGD.

In some embodiments, a PE6e prime editor comprises the amino acid sequence:

(SEQ ID NOs: 150, 151)
MKRTADGSEFESPKKKRKVDKKYSIGLDIGTNSVGWAVITDEYKVPSKKFKVLGNT
DRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAIL
SARLSKSRRLENLIAQLPGEKKNGLFGNLIALSLGLTPNFKSNFDLAEDAKLQLSKDT
YDDDLDNLLAQIGDQYADLFLAAKNLSDAILLSDILRVNTEITKAPLSASMIKRYDEH
HQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRD
KQSGKTILDFLKSDGFANRNFMQLIHDDSLTFKEDIQKAQVSGQGDSLHEHIANLAG
SPAIKKGILQTVKVVDELVKVMGRHKPENIVIEMARENQTTQKGQRNSRERMKRIEE
GIKELGSQILKEHPVENTQLQNEKLYLYYLQNGRDMYVDQELDINRLSDYDVDAIVP
QSFLKDDSIDNKVLTRSDKNRGKSDNVPSEEVVKKMKNYWRQLLNAKLITQRKFDN
LTKAERGGLSELDKAGFIARQLVETRQITKHVAQILDSRMNTKYDENDKLIREVKVIT
LKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDP
KKYGGFDSPTVAYSVLVVAKVEKGKSKKLKSVKELLGITIMERSSFEKNPIDFLEAK
GYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRD
KPIREQAENIIHLFTLTNLGAPAAFKYFDTTIDRKRYTSTKEVLDATLIHQSITGLYETR
IDLSQLGGDSGGSSGGSKRTADGSEFESPKKKRKVSGGSSGGS[REVERSE
TRANSCRIPTASEJKRTADGSEFESPKKKRKVPAAKRVKLD,
wherein [REVERSE TRANSCRIPTASE] comprises any reverse
transcriptase (e.g., any of the reverse transcriptase
variants disclosed herein).

In certain embodiments, the Cas9 variant comprises the amino acid sequence of SEQ ID NO: 48 (the Cas9 domain of “PE6f”), or an amino acid sequence at least 80%, at least 85%, at least 90%, at least 95%, at least 96%, at least 97%, at least 98%, or at least 99% identical to the amino acid sequence of SEQ ID NO: 48:

(SEQ ID NO: 48)
MDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFRRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEKTITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMVEE
RLKTYAHLFDNKVMKQLKRRRYTGWGRLSRKLINGIRDKQSGKTILDFLKSDGFAN
RNFMQLIHDDSLTFKEDIQKAQVSGQGDSLYEHIANLAGSPAIKKGILQTVKVVDEL
VKVMGRHKPENIVIEMARENQTTQKGQKNSRERMKRIEEGIKELGSQILKEHPVENT
QLQNEKLYLYYLQNGRDMYVDQELDINRLSDYDVDAIVPQSFLKDDSIDNKVLTRS
DKNRGKSDNVPSEEVVKKMKNYWRQLLNAKLITQRKFDNLTKAERGGLSELDKAG
FIARQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGD.

In some embodiments, a PE6f prime editor comprises the amino acid sequence:

(SEQ ID NOs: 152, 151)
MKRTADGSEFESPKKKRKVDKKYSIGLDIGTNSVGWAVITDEYKVPSKKFKVLGNT
DRHSIKKNLIGALLFDSGETAEATRLKRTARRRYTRRKNRICYLQEIFSNEMAKVDDS
FFRRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAIL
SARLSKSRRLENLIAQLPGEKKNGLFGNLIALSLGLTPNFKSNFDLAEDAKLQLSKDT
YDDDLDNLLAQIGDQYADLFLAAKNLSDAILLSDILRVNTEITKAPLSASMIKRYDEH
HQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKIL
TFRIPYYVGPLARGNSRFAWMTRKSEKTITPWNFEEVVDKGASAQSFIERMTNFDKN
LPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENEDI
LEDIVLTLTLFEDREMVEERLKTYAHLFDNKVMKQLKRRRYTGWGRLSRKLINGIR
DKQSGKTILDFLKSDGFANRNFMQLIHDDSLTFKEDIQKAQVSGQGDSLYEHIANLA
GSPAIKKGILQTVKVVDELVKVMGRHKPENIVIEMARENQTTQKGQKNSRERMKRIE
EGIKELGSQILKEHPVENTQLQNEKLYLYYLQNGRDMYVDQELDINRLSDYDVDAIV
PQSFLKDDSIDNKVLTRSDKNRGKSDNVPSEEVVKKMKNYWRQLLNAKLITQRKFD
NLTKAERGGLSELDKAGFIARQLVETRQITKHVAQILDSRMNTKYDENDKLIREVKV
ITLKSKLVSDFRKDFQFYKVREINNYHHAHDAYLNAVVGTALIKKYPKLESEFVYGD
YKVYDVRKMIAKSEQEIGKATAKYFFYSNIMNFFKTEITLANGEIRKRPLIETNGETG
EIVWDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYET
RIDLSQLGGDSGGSSGGSKRTADGSEFESPKKKRKVSGGSSGGS[REVERSE
TRANSCRIPTASEJKRTADGSEFESPKKKRKVPAAKRVKLD,
wherein [REVERSE TRANSCRIPTASE] comprises any reverse
transcriptase (e.g., any of the reverse transcriptase
variants disclosed herein).

In certain embodiments, the Cas9 variant comprises the amino acid sequence of SEQ ID NO: 49 (the Cas9 domain of “PE6g”), or an amino acid sequence at least 80%, at least 85%, at least 90%, at least 95%, at least 96%, at least 97%, at least 98%, or at least 99% identical to the amino acid sequence of SEQ ID NO: 49:

(SEQ ID NO: 49)
MDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFRRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEKTITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMVEE
RLKTYAHLFDNKVMKQLKRCRYTGWGRLSRKLINGIRDKQSGKTILDFLKSDGFAN
RNFMQLIHDDSLTFKEDIQKAQVSGQGDSLYEHIANLAGSPAIKKGILQTVKVVDEL
VKVMGRHKPENIVIEMARENQTTQKGQKNSRERMKRIEEGIKELGSQILKEHPVENT
QLQNEKLYLYYLQNGRDMYVDQELDINRLSDYDVDAIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGD.

In some embodiments, a PE6g prime editor comprises the amino acid sequence:

(SEQ ID NOs: 153, 151)
MKRTADGSEFESPKKKRKVDKKYSIGLDIGTNSVGWAVITDEYKVPSKKFKVLGNT
DRHSIKKNLIGALLFDSGETAEATRLKRTARRRYTRRKNRICYLQEIFSNEMAKVDDS
FFRRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAIL
SARLSKSRRLENLIAQLPGEKKNGLFGNLIALSLGLTPNFKSNFDLAEDAKLQLSKDT
YDDDLDNLLAQIGDQYADLFLAAKNLSDAILLSDILRVNTEITKAPLSASMIKRYDEH
HQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKIL
TFRIPYYVGPLARGNSRFAWMTRKSEKTITPWNFEEVVDKGASAQSFIERMTNFDKN
LPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENEDI
LEDIVLTLTLFEDREMVEERLKTYAHLFDNKVMKQLKRCRYTGWGRLSRKLINGIR
DKQSGKTILDFLKSDGFANRNFMQLIHDDSLTFKEDIQKAQVSGQGDSLYEHIANLA
GSPAIKKGILQTVKVVDELVKVMGRHKPENIVIEMARENQTTQKGQKNSRERMKRIE
EGIKELGSQILKEHPVENTQLQNEKLYLYYLQNGRDMYVDQELDINRLSDYDVDAIV
PQSFLKDDSIDNKVLTRSDKNRGKSDNVPSEEVVKKMKNYWRQLLNAKLITQRKFD
NLTKAERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESEFVYGD
YKVYDVRKMIAKSEQEIGKATAKYFFYSNIMNFFKTEITLANGEIRKRPLIETNGETG
EIVWDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYET
RIDLSQLGGDSGGSSGGSKRTADGSEFESPKKKRKVSGGSSGGS[REVERSE
TRANSCRIPTASEJKRTADGSEFESPKKKRKVPAAKRVKLD,
wherein [REVERSE TRANSCRIPTASE] comprises any reverse
transcriptase (e.g., any of the reverse transcriptase
variants disclosed herein).

In certain embodiments, the Cas9 variant comprises the amino acid sequence of SEQ ID NO: 145, or an amino acid sequence at least 80%, at least 85%, at least 90%, at least 95%, at least 96%, at least 97%, at least 98%, or at least 99% identical to the amino acid sequence of SEQ ID NO: 145:

(SEQ ID NO: 145)
MDKKYSIGLDIGTNSVGWAVITGEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDEL
VKVMGRRKPENIVIEMARENQTTQKGQKNSRERMKRIEEGIKELGSQILKEHPVENT
QLQNEKLYLYYLQNGRDMYVDQELDINRLSDYDVDAIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGD.

In some embodiments, any of the PE6 prime editors provided herein may comprise the architecture of PEmax. In some embodiments, any of the PE6 prime editors provided herein may comprise one or more additional amino acid substitutions (e.g., one, two, three, four, five, six, seven, eight, nine, or ten or more additional amino acid substitutions) relative to a wild type amino acid sequence, for example, any of the amino acid substitutions included in the Cas9 protein of PEmax or the MMLV reverse transcriptase of PEmax.

In some aspects, the present disclosure provides fusion proteins comprising any of the Cas9 variants provided herein and an effector domain. In certain embodiments, the effector domain comprises nuclease activity, nickase activity, recombinase activity, deaminase activity, methyltransferase activity, methylase activity, acetylase activity, acetyltransferase activity, transcriptional activation activity, transcriptional repression activity, or polymerase activity.

It should be appreciated that any of the amino acid mutations described herein, (e.g., E60K) from a first amino acid residue (e.g., E) to a second amino acid residue (e.g., K) may also include mutations from the first amino acid residue to an amino acid residue that is similar to (e.g., conserved) the second amino acid residue. For example, mutation of an amino acid with a hydrophobic side chain (e.g., alanine, valine, isoleucine, leucine, methionine, phenylalanine, tyrosine, or tryptophan) may be a mutation to a second amino acid with a different hydrophobic side chain (e.g., alanine, valine, isoleucine, leucine, methionine, phenylalanine, tyrosine, or tryptophan). For example, a mutation of an alanine to a threonine may also be a mutation from an alanine to an amino acid that is similar in size and chemical properties to a threonine, for example, serine. As another example, mutation of an amino acid with a positively charged side chain (e.g., arginine, histidine, or lysine) may be a mutation to a second amino acid with a different positively charged side chain (e.g., arginine, histidine, or lysine). As another example, mutation of an amino acid with a polar side chain (e.g., serine, threonine, asparagine, or glutamine) may be a mutation to a second amino acid with a different polar side chain (e.g., serine, threonine, asparagine, or glutamine). Additional similar amino acid pairs include, but are not limited to, the following: phenylalanine and tyrosine; asparagine and glutamine; methionine and cysteine; aspartic acid and glutamic acid; and arginine and lysine.

The skilled artisan would recognize that such conservative amino acid substitutions will likely have minor effects on protein structure and are likely to be well tolerated without compromising function. In some embodiments, any of the amino acid mutations provided herein from one amino acid to a threonine may be an amino acid mutation to a serine. In some embodiments, any of the amino acid mutations provided herein from one amino acid to an arginine may be an amino acid mutation to a lysine. In some embodiments, any of the amino acid mutations provided herein from one amino acid to an isoleucine may be an amino acid mutation to an alanine, valine, methionine, or leucine. In some embodiments, any of the amino acid mutations provided herein from one amino acid to a lysine may be an amino acid mutation to an arginine. In some embodiments, any of the amino acid mutations provided herein from one amino acid to an aspartic acid may be an amino acid mutation to a glutamic acid or asparagine. In some embodiments, any of the amino acid mutations provided herein from one amino acid to a valine may be an amino acid mutation to an alanine, isoleucine, methionine, or leucine. In some embodiments, any of the amino acid mutations provided herein from one amino acid to a glycine may be an amino acid mutation to an alanine. It should be appreciated, however, that additional conserved amino acid residues would be recognized by the skilled artisan, and any of the amino acid mutations to other conserved amino acid residues are also within the scope of this disclosure.

In some aspects, the present disclosure provides reverse transcriptase variants comprising mutations corresponding to any of the mutations disclosed herein, or any combination thereof, at a homologous position in another reverse transcriptase. Examples of additional reverse transcriptases include, but are not limited to, the following:

| | |
|---|---|
| MOUSE | VFTLWGRDIMKDIKVRLMTDSPDDSQDLMIGAIESNLFADQISW |
| MAMMARY | KSDQPVWLNQWPLKQEKLQALQQLVTEQLQLGHLEESNSPWN |
| TUMOR VIRUS | TPVFVIKKKSGKWRLLQDLRAVNATMHDMGALQPGLPSPVAVP |
| (MMTV) | KGWEIIIIDLQDCFFNIKLHPEDCKRFAFSVPSPNFKRPYQRFQWK |
| REVERSE | VLPQGMKNSPTLCQKFVDKAILTVRDKYQDSYIVHYMDDILLA |
| TRANSCRIPTASE | HPSRSIVDEILTSMIQALNKHGLVVSTEKIQKYDNLKYLGTHIQG |
| | DSVSYQKLQIRTDKLRTLNDFQKLLGNINWIRPFLKLTTGELKPL |
| | FEILNGDSNPISTRKLTPEACKALQLMNERLSTARVKRLDLSQPW |
| | SLCILKTEYTPTACLWQDGVVEWIHLPHISPKVITPYDIFCTQLIIK |
| | GRHRSKELFSKDPDYIVVPYTKVQFDLLLQEKEDWPISLLGFLGE |
| | VHFHLPKDPLLTFTLQTAIIFPHMTSTTPLEKGIVIFTDGSANGRS |
| | VTYIQGREPIIKENTQNTAQQAEIVAVITAFEEVSQPFNLYTDSKY |
| | VTGLFPEIETATLSPRTKIYTELKHLQRLIHKRQEKFYIGHIRGHT |
| | GLPGPLAQGNAYADSLTRILT (SEQ ID NO: 51) |
| AVIAN SARCOMA | TVALHLAIPLKWKPDHTPVWIDQWPLPEGKLVALTQLVEKELQ |
| LEUKOSIS VIRUS | LGHIEPSLSCWNTPVFVIRKASGSYRLLHDLRAVNAKLVPFGAV |
| (ASLV) REVERSE | QQGAPVLSALPRGWPLMVLDLKDCFFSIPLAEQDREAFAFTLPS |
| TRANSCRIPTASE | VNNQAPARRFQWKVLPQGMTCSPTICQLVVGQVLEPLRLKHPS |
| | LRMLHYMDDLLLAASSHDGLEAAGEEVISTLERAGFTISPDKIQR |
| | EPGVQYLGYKLGSTYVAPVGLVAEPRIATLWDVQKLVGSLQWL |
| | RPALGIPPRLMGPFYEQLRGSDPNEAREWNLDMKMAWREIVQL |
| | STTAALERWDPALPLEGAVARCEQGAIGVLGQGLSTHPRPCLWL |
| | FSTQPTKAFTAWLEVLTLLITKLRASAVRTFGKEVDILLLPACFR |
| | EDLPLPEGILLALKGFAGKIRSSDTPSIFDIARPLHVSLKVRVTDH |
| | PVPGPTVFTDASSSTHKGVVVWREGPRWEIKEIADSGASVQQLE |
| | ARAVAMALLLWPTTPTNVVTDSAFVAKMLLKMGQEGVPSTAA |
| | AFILEDALSQRSAMAAVLHVRSHSEVPGFFTEGNDVADSQATFQ |
| | AY (SEQ ID NO: 52) |
| PORCINE | TLQLDDEYRLYSPQVKPDQDIQSWLEQFPQAWAETAGMGLAKQ |
| ENDOGENOUS | VPPQVIQLKASATPVSVRQYPLSREAREGIWPHVQRLIQQGILVP |
| RETROVIRUS | VQSPWNTPLLPVRKPGTNDYRPVQDLREVNKRVQDIHPTVPNPY |
| (PERV) REVERSE | NLLSALPPERNWYTVLDLKDAFFCLRLHPTSQPLFAFEWRDPGT |
| TRANSCRIPTASE | GRTGQLTWTRLPQGFKNSPTIFDEALHRDLANFRIQHPQVTLLQ |
| | YVDDLLLAGATKQDCLEGTKALLLELSDLGYRASAKKAQICRR |
| | EVTYLGYSLRGGQRWLTEARKKTVVQIPAPTTAKQVREFLGTA |
| | GFCRLWIPGFATLAAPLYPLTKEKGEFSWAPEHQKAFDAIKKAL |
| | LSAPALALPDVTKPFTLYVDERKGVARGVLTQTLGPWRRPVAY |
| | LSKKLDPVASGWPVCLKAIAAVAILVKDADKLTLGQNITVIAPH |
| | ALENIVRQPPDRWMTNARMTHYQSLLLTERVTFAPPAALNPATL |
| | LPEETDEPVTHDCHQLLIEETGVRKDLTDIPLTGEVLTWFTDGSS |
| | YVVEGKRMAGAAVVDGTHTIWASSLPEGTSAQKAELMALTQA |
| | LRLAEGKSINIYTDSRYAFATAHVHGAIYKQRGLLTSAGREIKNK |
| | EEILSLLEALHLPKRLAIIHCPGHQKAKDLISRGNQMADRVAKQA |
| | AQAVNLLPI (SEQ ID NO: 53) |
| HIV-MMLV | PISPIETVPVKLKPGMDGPKVKQWPLTEEKIKALVEICTEMEKEG |
| REVERSE | KISKIGPENPYNTPVFAIKKKDSTKWRKLVDFRELNKRTQDFWE |
| TRANSCRIPTASE | VQLGIPHPAGLKKKKSVTVLDVGDAYFSVPLDEDFRKYTAFTIPS |
| | INNETPGIRYQYNVLPQGWKGSPAIFQSSMTKILEPFKKQNPDIVI |
| | YQYMDDLYVGSDLEIGQHRTKIEELRQHLLRWGLTTPDKKHQK |
| | EPPFLWMGYELHPDKWTVQPIVLPEKDSWTVNDIQKLVGKLNW |
| | ASQIYPGIKVRQLCKLLRGTKALTEVIPLTEEAELELAENREILKE |
| | PVHGVYYDPSKDLIAEIQKQGQGQWTYQIYQEPFKNLKTGKYA |
| | RMRGAHTNDVKQLTEAVQKITTESIVIWGKTPKFKLPIQKETWE |
| | TWWTEYWQATWIPEWEFVNTPPLVKLVVALNPATLLPLPEEGL |
| | QHNCLDILAEAHGTRPDLTDQPLPDADHTWYTDGSSLLQEGQR |
| | KAGAAVTTETEVIWAKALPAGTSAQRAELIALTQALKMAEGKK |
| | LNVYTDSRYAFATAHIHGEIYRRRGWLTSEGKEIKNKDEILALLK |
| | ALFLPKRLSIIHCPGHQKGHSAEARGNRMADQAARKAAITETPD |
| | TSTLLIEN (SEQ ID NO: 54) |
| AVIRE REVERSE | APLEEEYRLFLEAPIQNVTLLEQWKREIPKVWAEINPPGLASTQA |
| TRANSCRIPTASE | PIHVQLLSTALPVRVRQYPITLEAKRSLRETIRKFRAAGILRPVHS |
| | PWNTPLLPVRKSGTSEYRMVQDLREVNKRVETIHPTVPNPYTLL |
| | SLLPPDRIWYSVLDLKDAFFCIPLAPESQLIFAFEWADAEEGESG |
| | QLTWTRLPQGFKNSPTLFDEALNRDLQGFRLDHPSVSLLQYVDD |
| | LLIAADTQAACLSATRDLLMTLAELGYRVSGKKAQLCQEEVTY |
| | LGFKIHKGSRSLSNSRTQAILQIPVPKTKRQVREFLGTIGYCRLWI |
| | PGFAELAQPLYAATRGGNDPLVWGEKEEEAFQSLKLALTQPPAL |
| | ALPSLDKPFQLFVEETSGAAKGVLTQALGPWKRPVAYLSKRLDP |
| | VAAGWPRCLRAIAAAALLTREASKLTFGQDIEITSSHNLESLLRS |
| | PPDKWLTNARITQYQVLLLDPPRVRFKQTAALNPATLLPETDDT |
| | LPIHHCLDTLDSLTSTRPDLTDQPLAQAEATLFTDGSSYIRDGKR |
| | YAGAAVVTLDSVIWAEPLPIGTSAQKAELIALTKALEWSKDKSV |
| | NIYTDSRYAFATLHVHGMIYRERGLLTAGGKAIKNAPEILALLTA |
| | VWLPKRVAVMHCKGHQKDDAPTSTGNRRADEVAREVAIRPLST |
| | QATISDAPDMPDTETPQYSNVEEALG (SEQ ID NO: 55) |
| BABOON | VSLQDEHRLFDIPVTTSLPDVWLQDFPQAWAETGGLGRAKCQA |
| ENDOGENOUS | PIIIDLKPTAVPVSIKQYPMSLEAHMGIRQHIIKFLELGVLRPCRSP |
| VIRUS (BAEVM) | WNTPLLPVKKPGTQDYRPVQDLREINKRTVDIHPTVPNPYNLLS |
| REVERSE | TLKPDYSWYTVLDLKDAFFCLPLAPQSQELFAFEWKDPERGISG |
| TRANSCRIPTASE | QLTWTRLPQGFKNSPTLFDEALHRDLTDFRTQHPEVTLLQYVDD |
| | LLLAAPTKKACTQGTRHLLQELGEKGYRASAKKAQICQTKVTY |
| | LGYILSEGKRWLTPGRIETVARIPPPRNPREVREFLGTAGFCRLWI |
| | PGFAELAAPLYALTKESTPFTWQTEHQLAFEALKKALLSAPALG |
| | LPDTSKPFTLFLDERQGIAKGVLTQKLGPWKRPVAYLSKKLDPV |
| | AAGWPPCLRIMAATAMLVKDSAKLTLGQPLTVITPHTLEAIVRQ |
| | PPDRWITNARLTHYQALLLDTDRVQFGPPVTLNPATLLPVPENQ |
| | PSPHDCRQVLAETHGTREDLKDQELPDADHTWYTDGSSYLDSG |
| | TRRAGAAVVDGHNTIWAQSLPPGTSAQKAELIALTKALELSKGK |
| | KANIYTDSRYAFATAHTHGSIYERRGLLTSEGKEIKNKAEIIALLK |
| | ALFLPQEVAIIHCPGHQKGQDPVAVGNRQADRVARQAAMAEVL |
| | TLATEPDNTSHITIEHTYTSEDQEEA (SEQ ID NO: 56) |
| GIBBON APE | LNLEEEYRLHEKPVPSSIDPSWLQLFPTVWAERAGMGLANQVPP |
| LEUKEMIA | VVVELRSGASPVAVRQYPMSKEAREGIRPHIQKFLDLGVLVPCR |
| VIRUS (GALV) | SPWNTPLLPVKKPGTNDYRPVQDLREINKRVQDIHPTVPNPYNL |
| REVERSE | LSSLPPSYTWYSVLDLKDAFFCLRLHPNSQPLFAFEWKDPEKGN |
| TRANSCRIPTASE | TGQLTWTRLPQGFKNSPTLFDEALHRDLAPFRALNPQVVLLQYV |
| | DDLLVAAPTYEDCKKGTQKLLQELSKLGYRVSAKKAQLCQREV |
| | TYLGYLLKEGKRWLTPARKATVMKIPVPTTPRQVREFLGTAGFC |
| | RLWIPGFASLAAPLYPLTKESIPFIWTEEHQQAFDHIKKALLSAPA |
| | LALPDLTKPFTLYIDERAGVARGVLTQTLGPWRRPVAYLSKKLD |
| | PVASGWPTCLKAVAAVALLLKDADKLTLGQNVTVIASHSLESIV |
| | RQPPDRWMTNARMTHYQSLLLNERVSFAPPAVLNPATLLPVESE |
| | ATPVHRCSEILAEETGTRRDLEDQPLPGVPTWYTDGSSFITEGKR |
| | RAGAPIVDGKRTVWASSLPEGTSAQKAELVALTQALRLAEGKNI |
| | NIYTDSRYAFATAHIHGAIYKQRGLLTSAGKDIKNKEEILALLEAI |
| | HLPRRVAIIHCPGHQRGSNPVATGNRRADEAAKQAALSTRVLAG |
| | TTKPQEPIEPAQEK (SEQ ID NO: 57) |
| KOALA | MNLEEEYRLHEKPVPPSIDPSWLQLFPMVWAEKAGMGLANQVP |
| RETROVIRUS | PVVVELKSDASPVAVRQYPMSKEAREGIRPHIQRFLDLGILVPCQ |
| (KORV) REVERSE | SPWNTPLLPVKKPGTNDYRPVQDLREVNKRVQDIHPTVPNPYNL |
| TRANSCRIPTASE | LSSLPPSHTWYSVLDLKDAFFCLKLHPNSQPLFAFEWRDPEKGN |
| | TGQLTWTRLPQGFKNSPTLFDEALHRDLASFRALNPQVVMLQY |
| | VDDLLVAAPTYRDCKEGTRRLLQELSKLGYRVSAKKAQLCREE |
| | VTYLGYLLKGGKRWLTPARKATVMKIPTPTTPRQVREFLGTAGF |
| | CRLWIPGFASLAAPLYPLTREKVPFTWTEAHQEAFGRIKEALLSA |
| | PALALPDLTKPFALYVDEKEGVARGVLTQTLGPWRRPVAYLSK |
| | KLDPVASGWPTCLKAIAAVALLLKDADKLTLGQNVLVIAPHNL |
| | ESIVRQPPDRWMTNARMTHYQSLLLNERVSFAPPAILNPATLLP |
| | VESDDTPIHICSEILAEETGTRPDLRDQPLPGVPAWYTDGSSFIMD |
| | GRRQAGAAIVDNKRTVWASNLPEGTSAQKAELIALTQALRLAE |
| | GKSINIYTDSRYAFATAHVHGAIYKQRGLLTSAGKDIKNKEEILA |
| | LLEAIHLPKRVAIIHCPGHQRGTDPVATGNRKADEAAKQAAQST |
| | RILTETTKNQEHFEPTRGK (SEQ ID NO: 58) |
| MASON-PFIZER | MWGRDLLSQMKIMMCSPNDIVTAQMLAQGYSPGKGLGKKENG |
| MONKEY VIRUS | ILHPIPNQGQSNKKGFGNFLTAAIDILAPQQCAEPITWKSDEPVW |
| (MPMV) REVERSE | VDQWPLTNDKLAAAQQLVQEQLEAGHITESSSPWNTPIFVIKKK |
| TRANSCRIPTASE | SGKWRLLQDLRAVNATMVLMGALQPGLPSPVAIPQGYLKIIIDL |
| | KDCFFSIPLHPSDQKRFAFSLPSTNFKEPMQRFQWKVLPQGMAN |
| | SPTLCQKYVATAIHKVRHAWKQMYIIHYMDDILIAGKDGQQVL |
| | QCFDQLKQELTAAGLHIAPEKVQLQDPYTYLGFELNGPKITNQK |
| | AVIRKDKLQTLNDFQKLLGDINWLRPYLKLTTGDLKPLFDTLKG |
| | DSDPNSHRSLSKEALASLEKVETAIAEQFVTHINYSLPLIFLIFNTA |
| | LTPTGLFWQDNPIMWIHLPASPKKVLLPYYDAIADLIILGRDHSK |
| | KYFGIEPSTIIQPYSKSQIDWLMQNTEMWPIACASFVGILDNHYPP |
| | NKLIQFCKLHTFVFPQIISKTPLNNALLVFTDGSSTGMAAYTLTD |
| | TTIKFQTNLNSAQLVELQALIAVLSAFPNQPLNIYTDSAYLAHSIP |
| | LLETVAQIKHISETAKLFLQCQQLIYNRSIPFYIGHVRAHSGLPGPI |
| | AQGNQRADLATKIVA (SEQ ID NO: 59) |
| POK11ERV | ATVEPPKPIPLTWKTEKPVWVNQWPLPKQKLEALHLLANEQLE |
| REVERSE | KGHIEPSFSPWNSPVFVIQKKSGKWRMLTDLRAVNAVIQPMGPL |
| TRANSCRIPTASE | QPGLPSPAMIPKDWPLIIIDLKDCFFTIPLAEQDCEKFAFTIPAINN |
| | KEPATRFQWKVLPQGMLNSPTICQTFVGRALQPVREKFSDCYIIH |
| | YIDDILCAAETKDKLIDCYTFLQAEVANAGLAIASDKIQTSTPFH |
| | YLGMQIENRKIKPQKIEIRKDTLKTLNDFQKLLGDINWIRPTLGIP |
| | TYAMSNLFSILRGDSDLNSKRILTPEATKEIKLVEEKIQSAQINRID |
| | PLAPLQLLIFATAHSPTGIIIQNTDLVEWSFLPHSTVKTFTLYLDQI |
| | ATLIGQTRLRIIKLCGNDPDKIVVPLTKEQVRQAFINSGAWQIGL |
| | ANFVGIIDNHYPKTKIFQFLKMTTWILPKITRREPLENALTVFTDG |
| | SSNGKAAYTGPKERVIKTPYQSAQRAELVAVITVLQDFDQPINIIS |
| | DSAYVVQATRDVETALIKYSMDDQLNQLFNLLQQTVRKRNFPF |
| | YITHIRAHTNLPGPLTKANEEADLLVS (SEQ ID NO: 60) |
| SIMIAN | MWGRDLLSQMKIMMCSPNDIVTAQMLAQGYSPGKGLGKREDG |
| RETROVIRUS | ILQPIPNSGQLDRKGFGNFLATAVDILAPQRYADPITWKSDEPVW |
| TYPE 2 (SRV2) | VDQWPLTQEKLAAAQQLVQEQLQAGHIIESNSPWNTPIFVIKKK |
| REVERSE | SGKWRLLQDLRAVNATMVLMGALQPGLPSPVAIPQGYFKIVIDL |
| TRANSCRIPTASE | KDCFFTIPLQPVDQKRFAFSLPSTNFKQPMKRYQWKVLPQGMA |
| | NSPTLCQKYVAAAIEPVRKSWAQMYIIHYMDDILIAGKLGEQVL |
| | QCFAQLKQALTTTGLQIAPEKVQLQDPYTYLGFQINGPKITNQK |
| | AVIRRDKLQTLNDFQKLLGDINWLRPYLHLTTGDLKPLFDILKG |
| | DSNPNSPRSLSEAALASLQKVETAIAEQFVTQIDYTQPLTFLIFNT |
| | TLTPTGLFWQNNPVMWVHLPASPKKVLLPYYDAIADLIILGRDN |
| | SKKYFGLEPSTIIQPYSKSQIHWLMQNTETWPIACASYAGNIDNH |
| | YPPNKLIQFCKLHAVVFPRIISKTPLDNALLVFTDGSSTGIAAYTF |
| | EKTTVRFKTSHTSAQLVELQALIAVLSAFPHRALNVYTDSAYLA |
| | HSIPLLETVSHIKHISDTAKFFLQCQQLIYNRSIPFYLGHIRAHSGL |
| | PGPLSQGNHITDLATKVVA (SEQ ID NO: 61) |
| WOOLLY | LNLEEEYRLHEKPVPSSIDPSWLQLFPTVWAERAGMGLANQVPP |
| MONKEY | VVVELRSGASPVAVRQYPMSKEAREGIRPHIQRFLDLGVLVPCQ |
| SARCOMA VIRUS | SPWNTPLLPVKKPGTNDYRPVQDLREINKRVQDIHPTVPNPYNL |
| (WMSV) | LSSLPPSHTWYSVLDLKDAFFCLKLHPNSQPLFAFEWRDPEKGN |
| REVERSE | TGQLTWTRLPQGFKNSPTLFDEALHRDLAPFRALNPQVVLLQYV |
| TRANSCRIPTASE | DDLLVAAPTYRDCKEGTQKLLQELSKLGYRVSAKKAQLCQKEV |
| | TYLGYLLKEGKRWLTPARKATVMKIPPPTTPRQVREFLGTAGFC |
| | RLWIPGFASLAAPLYPLTKESIPFIWTEEHQKAFDRIKEALLSAPA |
| | LALPDLTKPFTLYVDERAGVARGVLTQTLGPWRRPVAYLSKKL |
| | DPVASGWPTCLKAVAAVALLLKDADKLTLGQNVTVIASHSLESI |
| | VRQPPDRWMTNARMTHYQSLLLNERVSFAPPAVLNPATLLPVE |
| | SEATPVHRCSEILAEETGTRRDLKDQPLPGVPAWYTDGSSFIAEG |
| | KRRAGAAIVDGKRTVWASSLPEGTSAQKAELVALTQALRLAEG |
| | KDINIYTDSRYAFATAHIHGAIYKQRGLLTSAGKDIKNKEEILAL |
| | LEAIHLPKRVAIIHCPGHQKGNDPVATGNRRADEAAKQAALSTR |
| | VLAETTKPQELI (SEQ ID NO: 62) |
| CRISPR REVERSE | NSQAQSACCAGANQIVEGATLEKVVAPACLQQAWTRVRKNKG |
| TRANSCRIPTASE | GPGGDGVTIEIFAQNAEVELEKLRAETLAGIYRPRKVRHAIVPKP |
| | KGGERKLTIPSVVDRILQTATMLSLGQTVDHHFSSASWAYREGR |
| | GVDDALADLRRLRNSGLFWTFDADIMQYFDRILHKRLIDDLFIW |
| | VDDLRIVRLIQLWLRSFSYWGRGIAQGAPISPLLANLFLHPMDRL |
| | LELEGLASVRYADDFVVLCRSKALAQKAQLIVASHLAARGLKL |
| | NMSKTRILAPSEAFIFLGQTVEPVWDTQP (SEQ ID NO: 63) |
| VP96 REVERSE | NLVKRLAHHLGKSEPEVIHFLADAPNKYRVYKIPKRSYGHRVIA |
| TRANSCRIPTASE | QPTRELKLYQKAFLELYSFPVHSSATAYCKGKSIKDNALSHVKN |
| | HYLLKTDLENFFNSITPNIFWKSIENDSIATPKFSTSEIALVERLIF |
| | WRPSKLQGGKLVLSVGAPSSPTISNFCLYQFDEYLSIICKEQNISY |
| | TRYADDLTFSTCDKDVLHTVIPLIQSLLDYFFASELKLNHSKTVF |
| | SSKAHNRHVTGITLNNEGKLSLGRERKRYIKHLVHSFKYGKLDN |
| | TEIRHLQGMLSFAKHIEPIFIDRLKEKYTDELIKIIYEAGHE (SEQ |
| | ID NO: 64) |
| VC95 REVERSE | NILTTLREQLLTNNVIMPQEFERLEVRGSHAYKVYSIPKRKAGRR |
| TRANSCRIPTASE | TIAHPSSKLKICQRHLNAILNPLLKVHDSSYAYVKGRSIKDNALV |
| | HSHSAYVLKMDFQNFFNSITPTILRQCLIQNDILLSVNELEKLEQL |
| | IFWNPSKKRNGKLILSVGSPISPLISNAIMYPFDKIINDICTKHGIN |
| | YTRYADDITFSTNIKNTLNKLPEIVEQLIIQTYAGRIIINKRKTVFS |
| | SKKHNRHVTGITLTNDSKISIGRSRKRYISSLVFKYINKNLDIDEIN |
| | HMKGMLAFAYNIEPIYIHRLSHKYKVNIVEKILRGSN (SEQ ID |
| | NO: 65) |
| GS REVERSE | ALLERILARDNLITALKRVEANQGAPGIDGVSTDQLRDYIRAHW |
| TRANSCRIPTASE | STIHAQLLAGTYRPAPVRRVEIPKPGGGTRQLGIPTVVDRLIQQAI |
| | LQELTPIFDPDFSSSSFGFRPGRNAHDAVRQAQGYIQEGYRYVVD |
| | MDLEKFFDRVNHDILMSRVARKVKDKRVLKLIRAYLQAGVMIE |
| | GVKVQTEEGTPQGGPLSPLLANILLDDLDKELEKRGLKFCRYAD |
| | DCNIYVKSLRAGQRVKQSIQRFLEKTLKLKVNEEKSAVDRPWK |
| | RAFLGFSFTPERKARIRLAPRSIQRLKQRIRQLTNPNWSISMPERI |
| | HRVNQYVMGWIGYFRLVETPSVLQTIEGWIRRRLRLCQWLQWK |
| | RVRTRIRELRALGLKETAVMEIANTRKGAWRTTKTPQLHQALG |
| | KTYWTAQGLKSLTQRYFELRQG (SEQ ID NO: 66) |
| ER REVERSE | DTSNLMEQILSSDNLNRAYLQVVRNKGAEGVDGMKYTELKEHL |
| TRANSCRIPTASE | AKNGETIKGQLRTRKYKPQPARRVEIPKPDGGVRNLGVPTVTDR |
| | FIQQAIAQVLTPIYEEQFHDHSYGFRPNRCAQQAILTALNIMNDG |
| | NDWIVDIDLEKFFDTVNHDKLMTLIGRTIKDGDVISIVRKYLVSG |
| | IMIDDEYEDSIVGTPQGGNLSPLLANIMLNELDKEMEKRGLNFV |
| | RYADDCIIMVGSEMSANRVMRNISRFIEEKLGLKVNMTKSKVDR |
| | PSGLKYLGFGFYFDPRAHQFKAKPHAKSVAKFKKRMKELTCRS |
| | WGVSNSYKVEKLNQLIRGWINYFKIGSMKTLCKELDSRIRYRLR |
| | MCIWKQWKTPQNQEKNLVKLGIDRNTARRVAYTGKRIAYVCN |
| | KGAVNVAISNKRLASFGLISMLDYYIEKCVTC (SEQ ID NO: 67) |
| NE144 REVERSE | AGQPTSREALYERIRSTSKEEVILEEMIRLGFWPAQGAVPHDPAE |
| TRANSCRIPTASE | EIRRRGELERQLSELREKSRKLYNEKALIAEQRKQRLAESRRKQK |
| | ETKARRERERQERAQKWAQRKAGEILFLGEDVSGGMSHKTCDA |
| | ELIKREGVPAIASAEELARAMGIALKELRFLAYNRKVSRVTHYR |
| | RFLLPKKTGGLRLISAPMPRLKRAQAWALEHIFNKLSFEPAAHGF |
| | VAGRSIVSNARPHVGADVVVNLDLKDFFPTVSFPRVKGALRHLG |
| | YSESVATALALVCTEPEVDEVGLDGTTWYVARGERFLPQGSPCS |
| | PAITNLLCRRLDRRLHGLAQALGFVYTRYADDLTFSGRGEAAES |
| | KRVGKLLRGAADIVAHEGFVVHPDKTRVMRRGRRQEVTGVVV |
| | NDKTSVPRDELRKFRATLYQIEKDGPADKRWGNGGDVLAAVH |
| | GYACFVAMVDPSRGQPLLARARALLAKHGGPSKPPGGSGPRAP |
| | TPVQPTANAPEAPKPVAPATPAAPAKKGWKLF (SEQ ID NO: 68) |
| M-MLV RT D200N | TLNIEDEYRLHETSKEPDVSLGSTWLSDFPQAWAETGGMGLAV |
| | RQAPLIIPLKATSTPVSIKQYPMSQEARLGIKPHIQRLLDQGILVPC |
| | QSPWNTPLLPVKKPGTNDYRPVQDLREVNKRVEDIHPTVPNPYN |
| | LLSGLPPSHQWYTVLDLKDAFFCLRLHPTSQPLFAFEWRDPEMG |
| | ISGQLTWTRLPQGFKNSPTLFNEALHRDLADFRIQHPDLILLQYV |
| | DDLLLAATSELDCQQGTRALLQTLGNLGYRASAKKAQICQKQV |
| | KYLGYLLKEGQRWLTEARKETVMGQPTPKTPRQLREFLGTAGF |
| | CRLWIPGFAEMAAPLYPLTKTGTLFNWGPDQQKAYQEIKQALL |
| | TAPALGLPDLTKPFELFVDEKQGYAKGVLTQKLGPWRRPVAYL |
| | SKKLDPVAAGWPPCLRMVAAIAVLTKDAGKLTMGQPLVILAPH |
| | AVEALVKQPPDRWLSNARMTHYQALLLDTDRVQFGPVVALNP |
| | ATLLPLPEEGLQHNCLDILAEAHGTRPDLTDQPLPDADHTWYTD |
| | GSSLLQEGQRKAGAAVTTETEVIWAKALPAGTSAQRAELIALTQ |
| | ALKMAEGKKLNVYTDSRYAFATAHIHGEIYRRRGLLTSEGKEIK |
| | NKDEILALLKALFLPKRLSIIHCPGHQKGHSAEARGNRMADQAA |
| | RKAAITETPDTSTLLIENSSP (SEQ ID NO: 32) |
| M-MLV RT D200N | TLNIEDEYRLHETSKEPDVSLGSTWLSDFPQAWAETGGMGLAV |
| T330P | RQAPLIIPLKATSTPVSIKQYPMSQEARLGIKPHIQRLLDQGILVPC |
| | QSPWNTPLLPVKKPGTNDYRPVQDLREVNKRVEDIHPTVPNPYN |
| | LLSGLPPSHQWYTVLDLKDAFFCLRLHPTSQPLFAFEWRDPEMG |
| | ISGQLTWTRLPQGFKNSPTLFNEALHRDLADFRIQHPDLILLQYV |
| | DDLLLAATSELDCQQGTRALLQTLGNLGYRASAKKAQICQKQV |
| | KYLGYLLKEGQRWLTEARKETVMGQPTPKTPRQLREFLGTAGF |
| | CRLWIPGFAEMAAPLYPLTKPGTLFNWGPDQQKAYQEIKQALLT |
| | APALGLPDLTKPFELFVDEKQGYAKGVLTQKLGPWRRPVAYLS |
| | KKLDPVAAGWPPCLRMVAAIAVLTKDAGKLTMGQPLVILAPHA |
| | VEALVKQPPDRWLSNARMTHYQALLLDTDRVQFGPVVALNPA |
| | TLLPLPEEGLQHNCLDILAEAHGTRPDLTDQPLPDADHTWYTDG |
| | SSLLQEGQRKAGAAVTTETEVIWAKALPAGTSAQRAELIALTQA |
| | LKMAEGKKLNVYTDSRYAFATAHIHGEIYRRRGLLTSEGKEIKN |
| | KDEILALLKALFLPKRLSIIHCPGHQKGHSAEARGNRMADQAAR |
| | KAAITETPDTSTLLIENSSP (SEQ ID NO: 33) |
| M-MLV RT D200N | TLNIEDEYRLHETSKEPDVSLGSTWLSDFPQAWAETGGMGLAV |
| T330P | RQAPLIIPLKATSTPVSIKQYPMSQEARLGIKPHIQRLLDQGILVPC |
| L603W | QSPWNTPLLPVKKPGTNDYRPVQDLREVNKRVEDIHPTVPNPYN |
| | LLSGLPPSHQWYTVLDLKDAFFCLRLHPTSQPLFAFEWRDPEMG |
| | ISGQLTWTRLPQGFKNSPTLFNEALHRDLADFRIQHPDLILLQYV |
| | DDLLLAATSELDCQQGTRALLQTLGNLGYRASAKKAQICQKQV |
| | KYLGYLLKEGQRWLTEARKETVMGQPTPKTPRQLREFLGTAGF |
| | CRLWIPGFAEMAAPLYPLTKPGTLFNWGPDQQKAYQEIKQALLT |
| | APALGLPDLTKPFELFVDEKQGYAKGVLTQKLGPWRRPVAYLS |
| | KKLDPVAAGWPPCLRMVAAIAVLTKDAGKLTMGQPLVILAPHA |
| | VEALVKQPPDRWLSNARMTHYQALLLDTDRVQFGPVVALNPA |
| | TLLPLPEEGLQHNCLDILAEAHGTRPDLTDQPLPDADHTWYTDG |
| | SSLLQEGQRKAGAAVTTETEVIWAKALPAGTSAQRAELIALTQA |
| | LKMAEGKKLNVYTDSRYAFATAHIHGEIYRRRGWLTSEGKEIK |
| | NKDEILALLKALFLPKRLSIIHCPGHQKGHSAEARGNRMADQAA |
| | RKAAITETPDTSTLLIENSSP (SEQ ID NO: 34) |
| M-MLV RT D200N | TLNIEDEYRLHETSKEPDVSLGSTWLSDFPQAWAETGGMGLAV |
| T330P | RQAPLIIPLKATSTPVSIKQYPMSQKARLGIKPHIQRLLDQGILVP |
| L603W | CQSPWNTPLLPVKKPGTNDYRPVQDLREVNKRVEDIHPTVPNPY |
| E69K | NLLSGLPPSHQWYTVLDLKDAFFCLRLHPTSQPLFAFEWRDPEM |
| | GISGQLTWTRLPQGFKNSPTLFNEALHRDLADFRIQHPDLILLQY |
| | VDDLLLAATSELDCQQGTRALLQTLGNLGYRASAKKAQICQKQ |
| | VKYLGYLLKEGQRWLTEARKETVMGQPTPKTPRQLREFLGTAG |
| | FCRLWIPGFAEMAAPLYPLTKPGTLFNWGPDQQKAYQEIKQALL |
| | TAPALGLPDLTKPFELFVDEKQGYAKGVLTQKLGPWRRPVAYL |
| | SKKLDPVAAGWPPCLRMVAAIAVLTKDAGKLTMGQPLVILAPH |
| | AVEALVKQPPDRWLSNARMTHYQALLLDTDRVQFGPVVALNP |
| | ATLLPLPEEGLQHNCLDILAEAHGTRPDLTDQPLPDADHTWYTD |
| | GSSLLQEGQRKAGAAVTTETEVIWAKALPAGTSAQRAELIALTQ |
| | ALKMAEGKKLNVYTDSRYAFATAHIHGEIYRRRGWLTSEGKEI |
| | KNKDEILALLKALFLPKRLSIIHCPGHQKGHSAEARGNRMADQA |
| | ARKAAITETPDTSTLLIENSSP (SEQ ID NO: 35) |
| M-MLV RT D200N | TLNIEDEYRLHETSKEPDVSLGSTWLSDFPQAWAETGGMGLAV |
| T330P | RQAPLIIPLKATSTPVSIKQYPMSQEARLGIKPHIQRLLDQGILVPC |
| L603W | QSPWNTPLLPVKKPGTNDYRPVQDLREVNKRVEDIHPTVPNPYN |
| E302R | LLSGLPPSHQWYTVLDLKDAFFCLRLHPTSQPLFAFEWRDPEMG |
| | ISGQLTWTRLPQGFKNSPTLFNEALHRDLADFRIQHPDLILLQYV |
| | DDLLLAATSELDCQQGTRALLQTLGNLGYRASAKKAQICQKQV |
| | KYLGYLLKEGQRWLTEARKETVMGQPTPKTPRQLRRFLGTAGF |
| | CRLWIPGFAEMAAPLYPLTKPGTLFNWGPDQQKAYQEIKQALLT |
| | APALGLPDLTKPFELFVDEKQGYAKGVLTQKLGPWRRPVAYLS |
| | KKLDPVAAGWPPCLRMVAAIAVLTKDAGKLTMGQPLVILAPHA |
| | VEALVKQPPDRWLSNARMTHYQALLLDTDRVQFGPVVALNPA |
| | TLLPLPEEGLQHNCLDILAEAHGTRPDLTDQPLPDADHTWYTDG |
| | SSLLQEGQRKAGAAVTTETEVIWAKALPAGTSAQRAELIALTQA |
| | LKMAEGKKLNVYTDSRYAFATAHIHGEIYRRRGWLTSEGKEIK |
| | NKDEILALLKALFLPKRLSIIHCPGHQKGHSAEARGNRMADQAA |
| | RKAAITETPDTSTLLIENSSP(SEQ ID NO: 36) |
| M-MLV RT D200N | TLNIEDEYRLHETSKEPDVSLGSTWLSDFPQAWAETGGMGLAV |
| T330P | RQAPLIIPLKATSTPVSIKQYPMSQEARLGIKPHIQRLLDQGILVPC |
| L603W | QSPWNTPLLPVKKPGTNDYRPVQDLREVNKRVEDIHPTVPNPYN |
| E607K | LLSGLPPSHQWYTVLDLKDAFFCLRLHPTSQPLFAFEWRDPEMG |
| | ISGQLTWTRLPQGFKNSPTLFNEALHRDLADFRIQHPDLILLQYV |
| | DDLLLAATSELDCQQGTRALLQTLGNLGYRASAKKAQICQKQV |
| | KYLGYLLKEGQRWLTEARKETVMGQPTPKTPRQLREFLGTAGF |
| | CRLWIPGFAEMAAPLYPLTKPGTLFNWGPDQQKAYQEIKQALLT |
| | APALGLPDLTKPFELFVDEKQGYAKGVLTQKLGPWRRPVAYLS |
| | KKLDPVAAGWPPCLRMVAAIAVLTKDAGKLTMGQPLVILAPHA |
| | VEALVKQPPDRWLSNARMTHYQALLLDTDRVQFGPVVALNPA |
| | TLLPLPEEGLQHNCLDILAEAHGTRPDLTDQPLPDADHTWYTDG |
| | SSLLQEGQRKAGAAVTTETEVIWAKALPAGTSAQRAELIALTQA |
| | LKMAEGKKLNVYTDSRYAFATAHIHGEIYRRRGWLTSKGKEIK |
| | NKDEILALLKALFLPKRLSIIHCPGHQKGHSAEARGNRMADQAA |
| | RKAAITETPDTSTLLIENSSP (SEQ ID NO: 37) |
| M-MLV RT D200N | TLNIEDEYRLHETSKEPDVSLGSTWLSDFPQAWAETGGMGLAV |
| T330P | RQAPLIIPLKATSTPVSIKQYPMSQEARLGIKPHIQRLLDQGILVPC |
| L603W | QSPWNTPLLPVKKPGTNDYRPVQDLREVNKRVEDIHPTVPNPYN |
| L139P | LLSGPPPSHQWYTVLDLKDAFFCLRLHPTSQPLFAFEWRDPEMG |
| | ISGQLTWTRLPQGFKNSPTLFNEALHRDLADFRIQHPDLILLQYV |
| | DDLLLAATSELDCQQGTRALLQTLGNLGYRASAKKAQICQKQV |
| | KYLGYLLKEGQRWLTEARKETVMGQPTPKTPRQLREFLGTAGF |
| | CRLWIPGFAEMAAPLYPLTKPGTLFNWGPDQQKAYQEIKQALLT |
| | APALGLPDLTKPFELFVDEKQGYAKGVLTQKLGPWRRPVAYLS |
| | KKLDPVAAGWPPCLRMVAAIAVLTKDAGKLTMGQPLVILAPHA |
| | VEALVKQPPDRWLSNARMTHYQALLLDTDRVQFGPVVALNPA |
| | TLLPLPEEGLQHNCLDILAEAHGTRPDLTDQPLPDADHTWYTDG |
| | SSLLQEGQRKAGAAVTTETEVIWAKALPAGTSAQRAELIALTQA |
| | LKMAEGKKLNVYTDSRYAFATAHIHGEIYRRRGWLTSEGKEIK |
| | NKDEILALLKALFLPKRLSIIHCPGHQKGHSAEARGNRMADQAA |
| | RKAAITETPDTSTLLIENSSP (SEQ ID NO: 38) |
| M-MLV RT D200N | TLNIEDEYRLHETSKEPDVSLGSTWLSDFPQAWAETGGMGLAV |
| T330P | RQAPLIIPLKATSTPVSIKQYPMSQEARLGIKPHIQRLLDQGILVPC |
| L603W | QSPWNTPLLPVKKPGTNDYRPVQDLREVNKRVEDIHPTVPNPYN |
| L435G | LLSGLPPSHQWYTVLDLKDAFFCLRLHPTSQPLFAFEWRDPEMG |
| | ISGQLTWTRLPQGFKNSPTLFNEALHRDLADFRIQHPDLILLQYV |
| | DDLLLAATSELDCQQGTRALLQTLGNLGYRASAKKAQICQKQV |
| | KYLGYLLKEGQRWLTEARKETVMGQPTPKTPRQLREFLGTAGF |
| | CRLWIPGFAEMAAPLYPLTKPGTLFNWGPDQQKAYQEIKQALLT |
| | APALGLPDLTKPFELFVDEKQGYAKGVLTQKLGPWRRPVAYLS |
| | KKLDPVAAGWPPCLRMVAAIAVLTKDAGKLTMGQPLVIGAPHA |
| | VEALVKQPPDRWLSNARMTHYQALLLDTDRVQFGPVVALNPA |
| | TLLPLPEEGLQHNCLDILAEAHGTRPDLTDQPLPDADHTWYTDG |
| | SSLLQEGQRKAGAAVTTETEVIWAKALPAGTSAQRAELIALTQA |
| | LKMAEGKKLNVYTDSRYAFATAHIHGEIYRRRGWLTSEGKEIK |
| | NKDEILALLKALFLPKRLSIIHCPGHQKGHSAEARGNRMADQAA |
| | RKAAITETPDTSTLLIENSSP (SEQ ID NO: 39) |
| M-MLV RT D200N | TLNIEDEYRLHETSKEPDVSLGSTWLSDFPQAWAETGGMGLAV |
| T330P | RQAPLIIPLKATSTPVSIKQYPMSQEARLGIKPHIQRLLDQGILVPC |
| L603W | QSPWNTPLLPVKKPGTNDYRPVQDLREVNKRVEDIHPTVPNPYN |
| N454K | LLSGLPPSHQWYTVLDLKDAFFCLRLHPTSQPLFAFEWRDPEMG |
| | ISGQLTWTRLPQGFKNSPTLFNEALHRDLADFRIQHPDLILLQYV |
| | DDLLLAATSELDCQQGTRALLQTLGNLGYRASAKKAQICQKQV |
| | KYLGYLLKEGQRWLTEARKETVMGQPTPKTPRQLREFLGTAGF |
| | CRLWIPGFAEMAAPLYPLTKPGTLFNWGPDQQKAYQEIKQALLT |
| | APALGLPDLTKPFELFVDEKQGYAKGVLTQKLGPWRRPVAYLS |
| | KKLDPVAAGWPPCLRMVAAIAVLTKDAGKLTMGQPLVILAPHA |
| | VEALVKQPPDRWLSKARMTHYQALLLDTDRVQFGPVVALNPA |
| | TLLPLPEEGLQHNCLDILAEAHGTRPDLTDQPLPDADHTWYTDG |
| | SSLLQEGQRKAGAAVTTETEVIWAKALPAGTSAQRAELIALTQA |
| | LKMAEGKKLNVYTDSRYAFATAHIHGEIYRRRGWLTSEGKEIK |
| | NKDEILALLKALFLPKRLSIIHCPGHQKGHSAEARGNRMADQAA |
| | RKAAITETPDTSTLLIENSSP (SEQ ID NO: 40) |
| M-MLV RT D200N | TLNIEDEYRLHETSKEPDVSLGSTWLSDFPQAWAETGGMGLAV |
| T330P | RQAPLIIPLKATSTPVSIKQYPMSQEARLGIKPHIQRLLDQGILVPC |
| L603W | QSPWNTPLLPVKKPGTNDYRPVQDLREVNKRVEDIHPTVPNPYN |
| T306K | LLSGLPPSHQWYTVLDLKDAFFCLRLHPTSQPLFAFEWRDPEMG |
| | ISGQLTWTRLPQGFKNSPTLFNEALHRDLADFRIQHPDLILLQYV |
| | DDLLLAATSELDCQQGTRALLQTLGNLGYRASAKKAQICQKQV |
| | KYLGYLLKEGQRWLTEARKETVMGQPTPKTPRQLREFLGKAGF |
| | CRLWIPGFAEMAAPLYPLTKPGTLFNWGPDQQKAYQEIKQALLT |
| | APALGLPDLTKPFELFVDEKQGYAKGVLTQKLGPWRRPVAYLS |
| | KKLDPVAAGWPPCLRMVAAIAVLTKDAGKLTMGQPLVILAPHA |
| | VEALVKQPPDRWLSNARMTHYQALLLDTDRVQFGPVVALNPA |
| | TLLPLPEEGLQHNCLDILAEAHGTRPDLTDQPLPDADHTWYTDG |
| | SSLLQEGQRKAGAAVTTETEVIWAKALPAGTSAQRAELIALTQA |
| | LKMAEGKKLNVYTDSRYAFATAHIHGEIYRRRGWLTSEGKEIK |
| | NKDEILALLKALFLPKRLSIIHCPGHQKGHSAEARGNRMADQAA |
| | RKAAITETPDTSTLLIENSSP (SEQ ID NO: 41) |
| M-MLV RT D200N | TLNIEDEYRLHETSKEPDVSLGSTWLSDFPQAWAETGGMGLAV |
| T330P | RQAPLIIPLKATSTPVSIKQYPMSQEARLGIKPHIQRLLDQGILVPC |
| L603W | QSPWNTPLLPVKKPGTNDYRPVQDLREVNKRVEDIHPTVPNPYN |
| W313F | LLSGLPPSHQWYTVLDLKDAFFCLRLHPTSQPLFAFEWRDPEMG |
| | ISGQLTWTRLPQGFKNSPTLFNEALHRDLADFRIQHPDLILLQYV |
| | DDLLLAATSELDCQQGTRALLQTLGNLGYRASAKKAQICQKQV |
| | KYLGYLLKEGQRWLTEARKETVMGQPTPKTPRQLREFLGTAGF |
| | CRLFIPGFAEMAAPLYPLTKPGTLFNWGPDQQKAYQEIKQALLT |
| | APALGLPDLTKPFELFVDEKQGYAKGVLTQKLGPWRRPVAYLS |
| | KKLDPVAAGWPPCLRMVAAIAVLTKDAGKLTMGQPLVILAPHA |
| | VEALVKQPPDRWLSNARMTHYQALLLDTDRVQFGPVVALNPA |
| | TLLPLPEEGLQHNCLDILAEAHGTRPDLTDQPLPDADHTWYTDG |
| | SSLLQEGQRKAGAAVTTETEVIWAKALPAGTSAQRAELIALTQA |
| | LKMAEGKKLNVYTDSRYAFATAHIHGEIYRRRGWLTSEGKEIK |
| | NKDEILALLKALFLPKRLSIIHCPGHQKGHSAEARGNRMADQAA |
| | RKAAITETPDTSTLLIENSSP (SEQ ID NO: 42) |
| M-MLV RT D200N | TLNIEDEYRLHETSKEPDVSLGSTWLSDFPQAWAETGGMGLAV |
| T330P | RQAPLIIPLKATSTPVSIKQYPMSQEARLGIKPHIQRLLDQGILVPC |
| L603W | QSPWNTPLLPVKKPGTNDYRPVQDLREVNKRVEDIHPTVPNPYN |
| D524G | LLSGLPPSHQWYTVLDLKDAFFCLRLHPTSQPLFAFEWRDPEMG |
| E562Q | ISGQLTWTRLPQGFKNSPTLFNEALHRDLADFRIQHPDLILLQYV |
| D583N | DDLLLAATSELDCQQGTRALLQTLGNLGYRASAKKAQICQKQV |
| | KYLGYLLKEGQRWLTEARKETVMGQPTPKTPRQLREFLGTAGF |
| | CRLWIPGFAEMAAPLYPLTKPGTLFNWGPDQQKAYQEIKQALLT |
| | APALGLPDLTKPFELFVDEKQGYAKGVLTQKLGPWRRPVAYLS |
| | KKLDPVAAGWPPCLRMVAAIAVLTKDAGKLTMGQPLVILAPHA |
| | VEALVKQPPDRWLSNARMTHYQALLLDTDRVQFGPVVALNPA |
| | TLLPLPEEGLQHNCLDILAEAHGTRPDLTDQPLPDADHTWYTGG |
| | SSLLQEGQRKAGAAVTTETEVIWAKALPAGTSAQRAQLIALTQA |
| | LKMAEGKKLNVYTNSRYAFATAHIHGEIYRRRGWLTSEGKEIK |
| | NKDEILALLKALFLPKRLSIIHCPGHQKGHSAEARGNRMADQAA |
| | RKAAITETPDTSTLLIENSSP (SEQ ID NO: 43) |
| M-MLV RT D200N | TLNIEDEYRLHETSKEPDVSLGSTWLSDFPQAWAETGGMGLAV |
| T330P | RQAPLIIPLKATSTPVSIKQYPMSQEARLGIKPHIQRLLDQGILVPC |
| L603W | QSPWNTPLLPVKKPGTNDYRPVQDLREVNKRVEDIHPTVPNPYN |
| E302R | LLSGLPPSHQWYTVLDLKDAFFCLRLHPTSQPLFAFEWRDPEMG |
| W313F | ISGQLTWTRLPQGFKNSPTLFNEALHRDLADFRIQHPDLILLQYV |
| | DDLLLAATSELDCQQGTRALLQTLGNLGYRASAKKAQICQKQV |
| | KYLGYLLKEGQRWLTEARKETVMGQPTPKTPRQLRRFLGTAGF |
| | CRLFIPGFAEMAAPLYPLTKPGTLFNWGPDQQKAYQEIKQALLT |
| | APALGLPDLTKPFELFVDEKQGYAKGVLTQKLGPWRRPVAYLS |
| | KKLDPVAAGWPPCLRMVAAIAVLTKDAGKLTMGQPLVILAPHA |
| | VEALVKQPPDRWLSNARMTHYQALLLDTDRVQFGPVVALNPA |
| | TLLPLPEEGLQHNCLDILAEAHGTRPDLTDQPLPDADHTWYTDG |
| | SSLLQEGQRKAGAAVTTETEVIWAKALPAGTSAQRAELIALTQA |
| | LKMAEGKKLNVYTDSRYAFATAHIHGEIYRRRGWLTSEGKEIK |
| | NKDEILALLKALFLPKRLSIIHCPGHQKGHSAEARGNRMADQAA |
| | RKAAITETPDTSTLLIENSSP (SEQ ID NO: 44) |
| M-MLV RT D200N | TLNIEDEYRLHETSKEPDVSLGSTWLSDFPQAWAETGGMGLAV |
| T330P | RQAPLIIPLKATSTPVSIKQYPMSQEARLGIKPHIQRLLDQGILVPC |
| L603W | QSPWNTPLLPVKKPGTNDYRPVQDLREVNKRVEDIHPTVPNPYN |
| E607K | LLSGPPPSHQWYTVLDLKDAFFCLRLHPTSQPLFAFEWRDPEMG |
| L139P | ISGQLTWTRLPQGFKNSPTLFNEALHRDLADFRIQHPDLILLQYV |
| | DDLLLAATSELDCQQGTRALLQTLGNLGYRASAKKAQICQKQV |
| | KYLGYLLKEGQRWLTEARKETVMGQPTPKTPRQLREFLGTAGF |
| | CRLWIPGFAEMAAPLYPLTKPGTLFNWGPDQQKAYQEIKQALLT |
| | APALGLPDLTKPFELFVDEKQGYAKGVLTQKLGPWRRPVAYLS |
| | KKLDPVAAGWPPCLRMVAAIAVLTKDAGKLTMGQPLVILAPHA |
| | VEALVKQPPDRWLSNARMTHYQALLLDTDRVQFGPVVALNPA |
| | TLLPLPEEGLQHNCLDILAEAHGTRPDLTDQPLPDADHTWYTDG |
| | SSLLQEGQRKAGAAVTTETEVIWAKALPAGTSAQRAELIALTQA |
| | LKMAEGKKLNVYTDSRYAFATAHIHGEIYRRRGWLTSKGKEIK |
| | NKDEILALLKALFLPKRLSIIHCPGHQKGHSAEARGNRMADQAA |
| | RKAAITETPDTSTLLIENSSP (SEQ ID NO: 45) |
| M-MLV RT P51L | TLNIEDEYRLHETSKEPDVSLGSTWLSDFPQAWAETGGMGLAV |
| S67K | RQAPLIILLKATSTPVSIKQYPMKQEARLGIKPHIQRLLDQGILVP |
| T197A H204R | CQSPWNTPLLPVKKPGTNDYRPVQDLREVNKRVEDIHPTVPNPY |
| E302K F309N | NLLSGLPPSHQWYTVLDLKDAFFCLRLHPTSQPLFAFEWRDPEM |
| W313F T330P | GISGQLTWTRLPQGFKNSPALFDEALRRDLADFRIQHPDLILLQY |
| L435G N454K | VDDLLLAATSELDCQQGTRALLQTLGNLGYRASAKKAQICQKQ |
| D524G D583N | VKYLGYLLKEGQRWLTEARKETVMGQPTPKTPRQLRKFLGTAG |
| H594Q D653N | NCRLFIPGFAEMAAPLYPLTKPGTLFNWGPDQQKAYQEIKQALL |
| | TAPALGLPDLTKPFELFVDEKQGYAKGVLTQKLGPWRRPVAYL |
| | SKKLDPVAAGWPPCLRMVAAIAVLTKDAGKLTMGQPLVIGAPH |
| | AVEALVKQPPDRWLSKARMTHYQALLLDTDRVQFGPVVALNP |
| | ATLLPLPEEGLQHNCLDILAEAHGTRPDLTDQPLPDADHTWYTG |
| | GSSLLQEGQRKAGAAVTTETEVIWAKALPAGTSAQRAELIALTQ |
| | ALKMAEGKKLNVYTNSRYAFATAHIQGEIYRRRGLLTSEGKEIK |
| | NKDEILALLKALFLPKRLSIIHCPGHQKGHSAEARGNRMANQAA |
| | RKAAITETPDTSTLLIENSSP (SEQ ID NO: 46) |
| M-MLV RT D200N | TLNIEDEYRLHETSKEPDVSLGSTWLSDFPQAWAETGGMGLAV |
| P51L | RQAPLIILLKATSTPVSIKQYPMKQEARLGIKPHIQRLLDQGILVP |
| S67K T197A | CQSPWNTPLLPVKKPGTNDYRPVQDLREVNKRVEDIHPTVPNPY |
| H204R E302K | NLLSGLPPSHQWYTVLDLKDAFFCLRLHPTSQPLFAFEWRDPEM |
| F309N W313F | GISGQLTWTRLPQGFKNSPALFNEALRRDLADFRIQHPDLILLQY |
| T330P L345G | VDDLLLAATSELDCQQGTRALLQTLGNLGYRASAKKAQICQKQ |
| N454K D524G | VKYLGYLLKEGQRWLTEARKETVMGQPTPKTPRQLRKFLGTAG |
| D583N H594Q | NCRLFIPGFAEMAAPLYPLTKPGTLFNWGPDQQKAYQEIKQALL |
| D653N | TAPALGLPDLTKPFELFVDEKQGYAKGVLTQKLGPWRRPVAYL |
| | SKKLDPVAAGWPPCLRMVAAIAVLTKDAGKLTMGQPLVIGAPH |
| | AVEALVKQPPDRWLSKARMTHYQALLLDTDRVQFGPVVALNP |
| | ATLLPLPEEGLQHNCLDILAEAHGTRPDLTDQPLPDADHTWYTG |
| | GSSLLQEGQRKAGAAVTTETEVIWAKALPAGTSAQRAELIALTQ |
| | ALKMAEGKKLNVYTNSRYAFATAHIQGEIYRRRGLLTSEGKEIK |
| | NKDEILALLKALFLPKRLSIIHCPGHQKGHSAEARGNRMANQAA |
| | RKAAITETPDTSTLLIENSSP (SEQ ID NO: 47) |
| M-MLV RT D200N | TLNIEDEYRLHETSKEPDVSLGSTWLSDFPQAWAETGGMGLAV |
| T330P L603W | RQAPLIIPLKATSTPVSIKQYPMSQEARLGIKPHIQRLLDQGILVPC |
| T306K W313F | QSPWNTPLLPVKKPGTNDYRPVQDLREVNKRVEDIHPTVPNPYN |
| (used in PE2 and | LLSGLPPSHQWYTVLDLKDAFFCLRLHPTSQPLFAFEWRDPEMG |
| PEmax) | ISGQLTWTRLPQGFKNSPTLFNEALHRDLADFRIQHPDLILLQYV |
| | DDLLLAATSELDCQQGTRALLQTLGNLGYRASAKKAQICQKQV |
| | KYLGYLLKEGQRWLTEARKETVMGQPTPKTPRQLREFLGKAGF |
| | CRLFIPGFAEMAAPLYPLTKPGTLFNWGPDQQKAYQEIKQALLT |
| | APALGLPDLTKPFELFVDEKQGYAKGVLTQKLGPWRRPVAYLS |
| | KKLDPVAAGWPPCLRMVAAIAVLTKDAGKLTMGQPLVILAPHA |
| | VEALVKQPPDRWLSNARMTHYQALLLDTDRVQFGPVVALNPA |
| | TLLPLPEEGLQHNCLDILAEAHGTRPDLTDQPLPDADHTWYTDG |
| | SSLLQEGQRKAGAAVTTETEVIWAKALPAGTSAQRAELIALTQA |
| | LKMAEGKKLNVYTDSRYAFATAHIHGEIYRRRGWLTSEGKEIK |
| | NKDEILALLKALFLPKRLSIIHCPGHQKGHSAEARGNRMADQAA |
| | RKAAITETPDTSTLLIENSSP (SEQ ID NO: 29) |

Additional reverse transcriptases are known in the art and will be readily apparent to those of skill in the art.

In some aspects, the present disclosure provides Cas9 variants comprising mutations corresponding to any of the mutations disclosed herein, or any combination thereof, at a homologous position in another Cas9 protein. Examples of additional Cas9 proteins include, but are not limited to, the following:

| | |
|---|---|
| Streptococcus | MDKKYSIGLDIGTNSVGWAVITDEYKVPSKKFKVLGNTDRHSIKKN |
| pyogenes Cas9 | LIGALLFDSGETAEATRLKRTARRRYTRRKNRICYLQEIFSNEMAKV |
| (Accession No. | DDSFFHRLEESFLVEEDKKHERHPIFGNIVDEVAYHEKYPTIYHLRK |
| Q99ZW2) | KLVDSTDKADLRLIYLALAHMIKFRGHFLIEGDLNPDNSDVDKLFIQ |
| | LVQTYNQLFEENPINASGVDAKAILSARLSKSRRLENLIAQLPGEKK |
| | NGLFGNLIALSLGLTPNFKSNFDLAEDAKLQLSKDTYDDDLDNLLA |
| | QIGDQYADLFLAAKNLSDAILLSDILRVNTEITKAPLSASMIKRYDEH |
| | HQDLTLLKALVRQQLPEKYKEIFFDQSKNGYAGYIDGGASQEEFYK |
| | FIKPILEKMDGTEELLVKLNREDLLRKQRTFDNGSIPHQIHLGELHAI |
| | LRRQEDFYPFLKDNREKIEKILTFRIPYYVGPLARGNSRFAWMTRKS |
| | EETITPWNFEEVVDKGASAQSFIERMTNFDKNLPNEKVLPKHSLLYE |
| | YFTVYNELTKVKYVTEGMRKPAFLSGEQKKAIVDLLFKTNRKVTV |
| | KQLKEDYFKKIECFDSVEISGVEDRFNASLGTYHDLLKIIKDKDFLD |
| | NEENEDILEDIVLTLTLFEDREMIEERLKTYAHLFDDKVMKQLKRRR |
| | YTGWGRLSRKLINGIRDKQSGKTILDFLKSDGFANRNFMQLIHDDSL |
| | TFKEDIQKAQVSGQGDSLHEHIANLAGSPAIKKGILQTVKVVDELVK |
| | VMGRHKPENIVIEMARENQTTQKGQKNSRERMKRIEEGIKELGSQIL |
| | KEHPVENTQLQNEKLYLYYLQNGRDMYVDQELDINRLSDYDVDHI |
| | VPQSFLKDDSIDNKVLTRSDKNRGKSDNVPSEEVVKKMKNYWRQL |
| | LNAKLITQRKFDNLTKAERGGLSELDKAGFIKRQLVETRQITKHVAQ |
| | ILDSRMNTKYDENDKLIREVKVITLKSKLVSDFRKDFQFYKVREINN |
| | YHHAHDAYLNAVVGTALIKKYPKLESEFVYGDYKVYDVRKMIAKS |
| | EQEIGKATAKYFFYSNIMNFFKTEITLANGEIRKRPLIETNGETGEIV |
| | WDKGRDFATVRKVLSMPQVNIVKKTEVQTGGFSKESILPKRNSDKL |
| | IARKKDWDPKKYGGFDSPTVAYSVLVVAKVEKGKSKKLKSVKELL |
| | GITIMERSSFEKNPIDFLEAKGYKEVKKDLIIKLPKYSLFELENGRKR |
| | MLASAGELQKGNELALPSKYVNFLYLASHYEKLKGSPEDNEQKQLF |
| | VEQHKHYLDEIIEQISEFSKRVILADANLDKVLSAYNKHRDKPIREQ |
| | AENIIHLFTLTNLGAPAAFKYFDTTIDRKRYTSTKEVLDATLIHQSIT |
| | GLYETRIDLSQLGGD (SEQ ID NO: 6) |
| Cas9 nickase | MDKKYSIGLDIGTNSVGWAVITDEYKVPSKKFKVLGNTDRHSIKKN |
| Streptococcus | LIGALLFDSGETAEATRLKRTARRRYTRRKNRICYLQEIFSNEMAKV |
| pyogenes Cas9 | DDSFFHRLEESFLVEEDKKHERHPIFGNIVDEVAYHEKYPTIYHLRK |
| (Accession No. | KLVDSTDKADLRLIYLALAHMIKFRGHFLIEGDLNPDNSDVDKLFIQ |
| Q99ZW2) with | LVQTYNQLFEENPINASGVDAKAILSARLSKSRRLENLIAQLPGEKK |
| H840A | NGLFGNLIALSLGLTPNFKSNFDLAEDAKLQLSKDTYDDDLDNLLA |
| | QIGDQYADLFLAAKNLSDAILLSDILRVNTEITKAPLSASMIKRYDEH |
| | HQDLTLLKALVRQQLPEKYKEIFFDQSKNGYAGYIDGGASQEEFYK |
| | FIKPILEKMDGTEELLVKLNREDLLRKQRTFDNGSIPHQIHLGELHAI |
| | LRRQEDFYPFLKDNREKIEKILTFRIPYYVGPLARGNSRFAWMTRKS |
| | EETITPWNFEEVVDKGASAQSFIERMTNFDKNLPNEKVLPKHSLLYE |
| | YFTVYNELTKVKYVTEGMRKPAFLSGEQKKAIVDLLFKTNRKVTV |
| | KQLKEDYFKKIECFDSVEISGVEDRFNASLGTYHDLLKIIKDKDFLD |
| | NEENEDILEDIVLTLTLFEDREMIEERLKTYAHLFDDKVMKQLKRRR |
| | YTGWGRLSRKLINGIRDKQSGKTILDFLKSDGFANRNFMQLIHDDSL |
| | TFKEDIQKAQVSGQGDSLHEHIANLAGSPAIKKGILQTVKVVDELVK |
| | VMGRHKPENIVIEMARENQTTQKGQKNSRERMKRIEEGIKELGSQIL |
| | KEHPVENTQLQNEKLYLYYLQNGRDMYVDQELDINRLSDYDVDAI |
| | VPQSFLKDDSIDNKVLTRSDKNRGKSDNVPSEEVVKKMKNYWRQL |
| | LNAKLITQRKFDNLTKAERGGLSELDKAGFIKRQLVETRQITKHVAQ |
| | ILDSRMNTKYDENDKLIREVKVITLKSKLVSDFRKDFQFYKVREINN |
| | YHHAHDAYLNAVVGTALIKKYPKLESEFVYGDYKVYDVRKMIAKS |
| | EQEIGKATAKYFFYSNIMNFFKTEITLANGEIRKRPLIETNGETGEIV |
| | WDKGRDFATVRKVLSMPQVNIVKKTEVQTGGFSKESILPKRNSDKL |
| | IARKKDWDPKKYGGFDSPTVAYSVLVVAKVEKGKSKKLKSVKELL |
| | GITIMERSSFEKNPIDFLEAKGYKEVKKDLIIKLPKYSLFELENGRKR |
| | MLASAGELQKGNELALPSKYVNFLYLASHYEKLKGSPEDNEQKQLF |
| | VEQHKHYLDEIIEQISEFSKRVILADANLDKVLSAYNKHRDKPIREQ |
| | AENIIHLFTLTNLGAPAAFKYFDTTIDRKRYTSTKEVLDATLIHQSIT |
| | GLYETRIDLSQLGGD (SEQ ID NO: 2) |
| SpCas9 | MDKKYSIGLDIGTNSVGWAVITDDYKVPSKKFKVLGNTDRHSIKKN |
| Streptococcus | LIGALLFGSGETAEATRLKRTARRRYTRRKNRICYLQEIFSNEMAKV |
| pyogenes | DDSFFHRLEESFLVEEDKKHERHPIFGNIVDEVAYHEKYPTIYHLRK |
| MGAS1882 wild | KLADSTDKADLRLIYLALAHMIKFRGHFLIEGDLNPDNSDVDKLFIQ |
| type NC_017053.1 | LVQIYNQLFEENPINASRVDAKAILSARLSKSRRLENLIAQLPGEKRN |
| | GLFGNLIALSLGLTPNFKSNFDLAEDAKLQLSKDTYDDDLDNLLAQI |
| | GDQYADLFLAAKNLSDAILLSDILRVNSEITKAPLSASMIKRYDEHH |
| | QDLTLLKALVRQQLPEKYKEIFFDQSKNGYAGYIDGGASQEEFYKFI |
| | KPILEKMDGTEELLVKLNREDLLRKQRTFDNGSIPHQIHLGELHAILR |
| | RQEDFYPFLKDNREKIEKILTFRIPYYVGPLARGNSRFAWMTRKSEE |
| | TITPWNFEEVVDKGASAQSFIERMTNFDKNLPNEKVLPKHSLLYEYF |
| | TVYNELTKVKYVTEGMRKPAFLSGEQKKAIVDLLFKTNRKVTVKQ |
| | LKEDYFKKIECFDSVEISGVEDRFNASLGAYHDLLKIIKDKDFLDNEE |
| | NEDILEDIVLTLTLFEDRGMIEERLKTYAHLFDDKVMKQLKRRRYT |
| | GWGRLSRKLINGIRDKQSGKTILDFLKSDGFANRNFMQLIHDDSLTF |
| | KEDIQKAQVSGQGHSLHEQIANLAGSPAIKKGILQTVKIVDELVKVM |
| | GHKPENIVIEMARENQTTQKGQKNSRERMKRIEEGIKELGSQILKEH |
| | PVENTQLQNEKLYLYYLQNGRDMYVDQELDINRLSDYDVDHIVPQ |
| | SFIKDDSIDNKVLTRSDKNRGKSDNVPSEEVVKKMKNYWRQLLNA |
| | KLITQRKFDNLTKAERGGLSELDKAGFIKRQLVETRQITKHVAQILD |
| | SRMNTKYDENDKLIREVKVITLKSKLVSDFRKDFQFYKVREINNYH |
| | HAHDAYLNAVVGTALIKKYPKLESEFVYGDYKVYDVRKMIAKSEQ |
| | EIGKATAKYFFYSNIMNFFKTEITLANGEIRKRPLIETNGETGEIVWD |
| | KGRDFATVRKVLSMPQVNIVKKTEVQTGGFSKESILPKRNSDKLIAR |
| | KKDWDPKKYGGFDSPTVAYSVLVVAKVEKGKSKKLKSVKELLGITI |
| | MERSSFEKNPIDFLEAKGYKEVKKDLIIKLPKYSLFELENGRKRMLA |
| | SAGELQKGNELALPSKYVNFLYLASHYEKLKGSPEDNEQKQLFVEQ |
| | HKHYLDEIIEQISEFSKRVILADANLDKVLSAYNKHRDKPIREQAENI |
| | IHLFTLTNLGAPAAFKYFDTTIDRKRYTSTKEVLDATLIHQSITGLYE |
| | TRIDLSQLGGD (SEQ ID NO: 8) |
| SpCas9 | QIGDQYADLFLAAKNLSDAILLSDILRVNTEITKAPLSASMIKRYDEH |
| Streptococcus | MDKKYSIGLDIGTNSVGWAVITDEYKVPSKKFKVLGNTDRHSIKKN |
| pyogenes wild | LIGALLFDSGETAEATRLKRTARRRYTRRKNRICYLQEIFSNEMAKV |
| type | DDSFFHRLEESFLVEEDKKHERHPIFGNIVDEVAYHEKYPTIYHLRK |
| Encoded product | KLVDSTDKADLRLIYLALAHMIKFRGHFLIEGDLNPDNSDVDKLFIQ |
| of | LVQTYNQLFEENPINASGVDAKAILSARLSKSRRLENLIAQLPGEKK |
| SWBC2D7W014 | NGLFGNLIALSLGLTPNFKSNFDLAEDAKLQLSKDTYDDDLDNLLA |
| | HQDLTLLKALVRQQLPEKYKEIFFDQSKNGYAGYIDGGASQEEFYK |
| | FIKPILEKMDGTEELLVKLNREDLLRKQRTFDNGSIPHQIHLGELHAI |
| | LRRQEDFYPFLKDNREKIEKILTFRIPYYVGPLARGNSRFAWMTRKS |
| | EETITPWNFEEVVDKGASAQSFIERMTNFDKNLPNEKVLPKHSLLYE |
| | YFTVYNELTKVKYVTEGMRKPAFLSGEQKKAIVDLLFKTNRKVTV |
| | KQLKEDYFKKIECFDSVEISGVEDRFNASLGTYHDLLKIIKDKDFLD |
| | NEENEDILEDIVLTLTLFEDREMIEERLKTYAHLFDDKVMKQLKRRR |
| | YTGWGRLSRKLINGIRDKQSGKTILDFLKSDGFANRNFMQLIHDDSL |
| | TFKEDIQKAQVSGQGDSLHEHIANLAGSPAIKKGILQTVKVVDELVK |
| | VMGRHKPENIVIEMARENQTTQKGQKNSRERMKRIEEGIKELGSQIL |
| | KEHPVENTQLQNEKLYLYYLQNGRDMYVDQELDINRLSDYDVDHI |
| | VPQSFLKDDSIDNKVLTRSDKNRGKSDNVPSEEVVKKMKNYWRQL |
| | LNAKLITQRKFDNLTKAERGGLSELDKAGFIKRQLVETRQITKHVAQ |
| | ILDSRMNTKYDENDKLIREVKVITLKSKLVSDFRKDFQFYKVREINN |
| | YHHAHDAYLNAVVGTALIKKYPKLESEFVYGDYKVYDVRKMIAKS |
| | EQEIGKATAKYFFYSNIMNFFKTEITLANGEIRKRPLIETNGETGEIV |
| | WDKGRDFATVRKVLSMPQVNIVKKTEVQTGGFSKESILPKRNSDKL |
| | IARKKDWDPKKYGGFDSPTVAYSVLVVAKVEKGKSKKLKSVKELL |
| | GITIMERSSFEKNPIDFLEAKGYKEVKKDLIIKLPKYSLFELENGRKR |
| | MLASAGELQKGNELALPSKYVNFLYLASHYEKLKGSPEDNEQKQLF |
| | VEQHKHYLDEIIEQISEFSKRVILADANLDKVLSAYNKHRDKPIREQ |
| | AENIIHLFTLTNLGAPAAFKYFDTTIDRKRYTSTKEVLDATLIHQSIT |
| | GLYETRIDLSQLGGDGSPKKKRKVSSDYKDHDGDYKDHDIDYKDD |
| | DDKAAG (SEQ ID NO: 9) |
| SpCas9 | MDKKYSIGLDIGTNSVGWAVITDEYKVPSKKFKVLGNTDRHSIKKN |
| Streptococcus | LIGALLFDSGETAEATRLKRTARRRYTRRKNRICYLQEIFSNEMAKV |
| pyogenes | DDSFFHRLEESFLVEEDKKHERHPIFGNIVDEVAYHEKYPTIYHLRK |
| MIGAS wild | KLVDSTDKADLRLIYLALAHMIKFRGHFLIEGDLNPDNSDVDKLFIQ |
| type | LVQTYNQLFEENPINASGVDAKAILSARLSKSRRLENLIAQLPGEKK |
| | NGLFGNLIALSLGLTPNFKSNFDLAEDAKLQLSKDTYDDDLDNLLA |
| | QIGDQYADLFLAAKNLSDAILLSDILRVNTEITKAPLSASMIKRYDEH |
| | HQDLTLLKALVRQQLPEKYKEIFFDQSKNGYAGYIDGGASQEEFYK |
| | FIKPILEKMDGTEELLVKLNREDLLRKQRTFDNGSIPHQIHLGELHAI |
| | LRRQEDFYPFLKDNREKIEKILTFRIPYYVGPLARGNSRFAWMTRKS |
| | EETITPWNFEEVVDKGASAQSFIERMTNFDKNLPNEKVLPKHSLLYE |
| | YFTVYNELTKVKYVTEGMRKPAFLSGEQKKAIVDLLFKTNRKVTV |
| | KQLKEDYFKKIECFDSVEISGVEDRFNASLGTYHDLLKIIKDKDFLD |
| | NEENEDILEDIVLTLTLFEDREMIEERLKTYAHLFDDKVMKQLKRRR |
| | YTGWGRLSRKLINGIRDKQSGKTILDFLKSDGFANRNFMQLIHDDSL |
| | TFKEDIQKAQVSGQGDSLHEHIANLAGSPAIKKGILQTVKVVDELVK |
| | VMGRHKPENIVIEMARENQTTQKGQKNSRERMKRIEEGIKELGSQIL |
| | KEHPVENTQLQNEKLYLYYLQNGRDMYVDQELDINRLSDYDVDHI |
| | VPQSFLKDDSIDNKVLTRSDKNRGKSDNVPSEEVVKKMKNYWRQL |
| | LNAKLITQRKFDNLTKAERGGLSELDKAGFIKRQLVETRQITKHVAQ |
| | ILDSRMNTKYDENDKLIREVKVITLKSKLVSDFRKDFQFYKVREINN |
| | YHHAHDAYLNAVVGTALIKKYPKLESEFVYGDYKVYDVRKMIAKS |
| | EQEIGKATAKYFFYSNIMNFFKTEITLANGEIRKRPLIETNGETGEIV |
| | WDKGRDFATVRKVLSMPQVNIVKKTEVQTGGFSKESILPKRNSDKL |
| | IARKKDWDPKKYGGFDSPTVAYSVLVVAKVEKGKSKKLKSVKELL |
| | GITIMERSSFEKNPIDFLEAKGYKEVKKDLIIKLPKYSLFELENGRKR |
| | MLASAGELQKGNELALPSKYVNFLYLASHYEKLKGSPEDNEQKQLF |
| | VEQHKHYLDEIIEQISEFSKRVILADANLDKVLSAYNKHRDKPIREQ |
| | AENIIHLFTLTNLGAPAAFKYFDTTIDRKRYTSTKEVLDATLIHQSIT |
| | GLYETRIDLSQLGGD (SEQ ID NO: 6) |
| LfCas9 | MKEYHIGLDIGTSSIGWAVTDSQFKLMRIKGKTAIGVRLFEEGKTAA |
| Lactobacillus | ERRTFRTTRRRLKRRKWRLHYLDEIFAPHLQEVDENFLRRLKQSNIH |
| fermentum wild | PEDPTKNQAFIGKLLFPDLLKKNERGYPTLIKMRDELPVEQRAHYPV |
| type | MNIYKLREAMINEDRQFDLREVYLAVHHIVKYRGHFLNNASVDKF |
| GenBank: | KVGRIDFDKSFNVLNEAYEELQNGEGSFTIEPSKVEKIGQLLLDTKM |
| SNX31424.1 1 | RKLDRQKAVAKLLEVKVADKEETKRNKQIATAMSKLVLGYKADF |
| | ATVAMANGNEWKIDLSSETSEDEIEKFREELSDAQNDILTEITSLFSQ |
| | IMLNEIVPNGMSISESMMDRYWTHERQLAEVKEYLATQPASARKEF |
| | DQVYNKYIGQAPKERGFDLEKGLKKILSKKENWKEIDELLKAGDFL |
| | PKQRTSANGVIPHQMHQQELDRIIEKQAKYYPWLATENPATGERDR |
| | HQAKYELDQLVSFRIPYYVGPLVTPEVQKATSGAKFAWAKRKEDG |
| | EITPWNLWDKIDRAESAEAFIKRMTVKDTYLLNEDVLPANSLLYQK |
| | YNVLNELNNVRVNGRRLSVGIKQDIYTELFKKKKTVKASDVASLV |
| | MAKTRGVNKPSVEGLSDPKKFNSNLATYLDLKSIVGDKVDDNRYQ |
| | TDLENIIEWRSVFEDGEIFADKLTEVEWLTDEQRSALVKKRYKGWG |
| | RLSKKLLTGIVDENGQRIIDLMWNTDQNFKEIVDQPVFKEQIDQLNQ |
| | KAITNDGMTLRERVESVLDDAYTSPQNKKAIWQVVRVVEDIVKAV |
| | GNAPKSISIEFARNEGNKGEITRSRRTQLQKLFEDQAHELVKDTSLTE |
| | ELEKAPDLSDRYYFYFTQGGKDMYTGDPINFDEISTKYDIDHILPQSF |
| | VKDNSLDNRVLTSRKENNKKSDQVPAKLYAAKMKPYWNQLLKQG |
| | LITQRKFENLTKDVDQNIKYRSLGFVKRQLVETRQVIKLTANILGSM |
| | YQEAGTEIIETRAGLTKQLREEFDLPKVREVNDYHHAVDAYLTTFA |
| | GQYLNRRYPKLRSFFVYGEYMKFKHGSDLKLRNFNFFHELMEGDK |
| | SQGKVVDQQTGELITTRDEVAKSFDRLLNMKYMLVSKEVHDRSDQ |
| | LYGATIVTAKESGKLTSPIEIKKNRLVDLYGAYTNGTSAFMTIIKFTG |
| | NKPKYKVIGIPTTSAASLKRAGKPGSESYNQELHRIIKSNPKVKKGFE |
| | IVVPHVSYGQLIVDGDCKFTLASPTVQHPATQLVLSKKSLETISSGY |
| | KILKDKPAIANERLIRVFDEVVGQMNRYFTIFDQRSNRQKVADARD |
| | KFLSLPTESKYEGAKKVQVGKTEVITNLLMGLHANATQGDLKVLG |
| | LATFGFFQSTTGLSLSEDTMIVYQSPTGLFERRICLKDI (SEQ ID NO: |
| | 12) |
| SaCas9 | MDKKYSIGLDIGTNSVGWAVITDEYKVPSKKFKVLGNTDRHSIKKN |
| Staphylococcus | LIGALLFDSGETAEATRLKRTARRRYTRRKNRICYLQEIFSNEMAKV |
| aureus wild type | DDSFFHRLEESFLVEEDKKHERHPIFGNIVDEVAYHEKYPTIYHLRK |
| GenBank: | KLVDSTDKADLRLIYLALAHMIKFRGHFLIEGDLNPDNSDVDKLFIQ |
| AYD60528.1 | LVQTYNQLFEENPINASGVDAKAILSARLSKSRRLENLIAQLPGEKK |
| | NGLFGNLIALSLGLTPNFKSNFDLAEDAKLQLSKDTYDDDLDNLLA |
| | QIGDQYADLFLAAKNLSDAILLSDILRVNTEITKAPLSASMIKRYDEH |
| | HQDLTLLKALVRQQLPEKYKEIFFDQSKNGYAGYIDGGASQEEFYK |
| | FIKPILEKMDGTEELLVKLNREDLLRKQRTFDNGSIPHQIHLGELHAI |
| | LRRQEDFYPFLKDNREKIEKILTFRIPYYVGPLARGNSRFAWMTRKS |
| | EETITPWNFEEVVDKGASAQSFIERMTNFDKNLPNEKVLPKHSLLYE |
| | YFTVYNELTKVKYVTEGMRKPAFLSGEQKKAIVDLLFKTNRKVTV |
| | KQLKEDYFKKIECFDSVEISGVEDRFNASLGTYHDLLKIIKDKDFLD |
| | NEENEDILEDIVLTLTLFEDREMIEERLKTYAHLFDDKVMKQLKRRR |
| | YTGWGRLSRKLINGIRDKQSGKTILDFLKSDGFANRNFMQLIHDDSL |
| | TFKEDIQKAQVSGQGDSLHEHIANLAGSPAIKKGILQTVKVVDELVK |
| | VMGRHKPENIVIEMARENQTTQKGQKNSRERMKRIEEGIKELGSQIL |
| | KEHPVENTQLQNEKLYLYYLQNGRDMYVDQELDINRLSDYDVDHI |
| | VPQSFLKDDSIDNKVLTRSDKNRGKSDNVPSEEVVKKMKNYWRQL |
| | LNAKLITQRKFDNLTKAERGGLSELDKAGFIKRQLVETRQITKHVAQ |
| | ILDSRMNTKYDENDKLIREVKVITLKSKLVSDFRKDFQFYKVREINN |
| | YHHAHDAYLNAVVGTALIKKYPKLESEFVYGDYKVYDVRKMIAKS |
| | EQEIGKATAKYFFYSNIMNFFKTEITLANGEIRKRPLIETNGETGEIV |
| | WDKGRDFATVRKVLSMPQVNIVKKTEVQTGGFSKESILPKRNSDKL |
| | IARKKDWDPKKYGGFDSPTVAYSVLVVAKVEKGKSKKLKSVKELL |
| | GITIMERSSFEKNPIDFLEAKGYKEVKKDLIIKLPKYSLFELENGRKR |
| | MLASAGELQKGNELALPSKYVNFLYLASHYEKLKGSPEDNEQKQLF |
| | VEQHKHYLDEIIEQISEFSKRVILADANLDKVLSAYNKHRDKPIREQ |
| | AENIIHLFTLTNLGAPAAFKYFDTTIDRKRYTSTKEVLDATLIHQSIT |
| | GLYETRIDLSQLGGD (SEQ ID NO: 6) |
| SaCas9 | MGKRNYILGLDIGITSVGYGIIDYETRDVIDAGVRLFKEANVENNEG |
| Staphylococcus | RRSKRGARRLKRRRRHRIQRVKKLLFDYNLLTDHSELSGINPYEAR |
| aureus | VKGLSQKLSEEEFSAALLHLAKRRGVHNVNEVEEDTGNELSTKEQI |
| | SRNSKALEEKYVAELQLERLKKDGEVRGSINRFKTSDYVKEAKQLL |
| | KVQKAYHQLDQSFIDTYIDLLETRRTYYEGPGEGSPFGWKDIKEWY |
| | EMLMGHCTYFPEELRSVKYAYNADLYNALNDLNNLVITRDENEKL |
| | EYYEKFQIIENVFKQKKKPTLKQIAKEILVNEEDIKGYRVTSTGKPEF |
| | TNLKVYHDIKDITARKEIIENAELLDQIAKILTIYQSSEDIQEELTNLN |
| | SELTQEEIEQISNLKGYTGTHNLSLKAINLILDELWHTNDNQIAIFNR |
| | LKLVPKKVDLSQQKEIPTTLVDDFILSPVVKRSFIQSIKVINAIIKKYG |
| | LPNDIIIELAREKNSKDAQKMINEMQKRNRQTNERIEEIIRTTGKENA |
| | KYLIEKIKLHDMQEGKCLYSLEAIPLEDLLNNPFNYEVDHIIPRSVSF |
| | DNSFNNKVLVKQEENSKKGNRTPFQYLSSSDSKISYETFKKHILNLA |
| | KGKGRISKTKKEYLLEERDINRFSVQKDFINRNLVDTRYATRGLMN |
| | LLRSYFRVNNLDVKVKSINGGFTSFLRRKWKFKKERNKGYKHHAE |
| | DALIIANADFIFKEWKKLDKAKKVMENQMFEEKQAESMPEIETEQE |
| | YKEIFITPHQIKHIKDFKDYKYSHRVDKKPNRKLINDTLYSTRKDDK |
| | GNTLIVNNLNGLYDKDNDKLKKLINKSPEKLLMYHHDPQTYQKLK |
| | LIMEQYGDEKNPLYKYYEETGNYLTKYSKKDNGPVIKKIKYYGNKL |
| | NAHLDITDDYPNSRNKVVKLSLKPYRFDVYLDNGVYKFVTVKNLD |
| | VIKKENYYEVNSKCYEEAKKLKKISNQAEFIASFYKNDLIKINGELY |
| | RVIGVNNDLLNRIEVNMIDITYREYLENMNDKRPPHIIKTIASKTQSI |
| | KKYSTDILGNLYEVKSKKHPQIIKK (SEQ ID NO: 13) |
| StCas9 | MLFNKCIIISINLDFSNKEKCMTKPYSIGLDIGTNSVGWAVITDNYKV |
| Streptococcus | PSKKMKVLGNTSKKYIKKNLLGVLLFDSGITAEGRRLKRTARRRYT |
| thermophilus | RRRNRILYLQEIFSTEMATLDDAFFQRLDDSFLVPDDKRDSKYPIFG |
| UniProtKB/ | NLVEEKVYHDEFPTIYHLRKYLADSTKKADLRLVYLALAHMIKYRG |
| Swiss-Prot: | HFLIEGEFNSKNNDIQKNFQDFLDTYNAIFESDLSLENSKQLEEIVKD |
| G3ECR1.2 | KISKLEKKDRILKLFPGEKNSGIFSEFLKLIVGNQADFRKCFNLDEKA |
| Wild type | SLHFSKESYDEDLETLLGYIGDDYSDVFLKAKKLYDAILLSGFLTVT |
| | DNETEAPLSSAMIKRYNEHKEDLALLKEYIRNISLKTYNEVFKDDTK |
| | NGYAGYIDGKTNQEDFYVYLKNLLAEFEGADYFLEKIDREDFLRKQ |
| | RTFDNGSIPYQIHLQEMRAILDKQAKFYPFLAKNKERIEKILTFRIPY |
| | YVGPLARGNSDFAWSIRKRNEKITPWNFEDVIDKESSAEAFINRMTS |
| | FDLYLPEEKVLPKHSLLYETFNVYNELTKVRFIAESMRDYQFLDSKQ |
| | KKDIVRLYFKDKRKVTDKDIIEYLHAIYGYDGIELKGIEKQFNSSLST |
| | YHDLLNIINDKEFLDDSSNEAIIEEIIHTLTIFEDREMIKQRLSKFENIF |
| | DKSVLKKLSRRHYTGWGKLSAKLINGIRDEKSGNTILDYLIDDGISN |
| | RNFMQLIHDDALSFKKKIQKAQIIGDEDKGNIKEVVKSLPGSPAIKK |
| | GILQSIKIVDELVKVMGGRKPESIVVEMARENQYTNQGKSNSQQRL |
| | KRLEKSLKELGSKILKENIPAKLSKIDNNALQNDRLYLYYLQNGKD |
| | MYTGDDLDIDRLSNYDIDHIIPQAFLKDNSIDNKVLVSSASNRGKSD |
| | DFPSLEVVKKRKTFWYQLLKSKLISQRKFDNLTKAERGGLLPEDKA |
| | GFIQRQLVETRQITKHVARLLDEKFNNKKDENNRAVRTVKIITLKST |
| | LVSQFRKDFELYKVREINDFHHAHDAYLNAVIASALLKKYPKLEPEF |
| | VYGDYPKYNSFRERKSATEKVYFYSNIMNIFKKSISLADGRVIERPLI |
| | EVNEETGESVWNKESDLATVRRVLSYPQVNVVKKVEEQNHGLDRG |
| | KPKGLFNANLSSKPKPNSNENLVGAKEYLDPKKYGGYAGISNSFAV |
| | LVKGTIEKGAKKKITNVLEFQGISILDRINYRKDKLNFLLEKGYKDIE |
| | LIIELPKYSLFELSDGSRRMLASILSTNNKRGEIHKGNQIFLSQKFVKL |
| | LYHAKRISNTINENHRKYVENHKKEFEELFYYILEFNENYVGAKKN |
| | GKLLNSAFQSWQNHSIDELCSSFIGPTGSERKGLFELTSRGSAADFEF |
| | LGVKIPRYRDYTPSSLLKDATLIHQSVTGLYETRIDLAKLGEG (SEQ |
| | ID NO: 14) |
| LcCas9 | MKIKNYNLALTPSTSAVGHVEVDDDLNILEPVHHQKAIGVAKFGEG |
| Lactobacillus | ETAEARRLARSARRTTKRRANRINHYFNEIMKPEIDKVDPLMFDRIK |
| crispatus | QAGLSPLDERKEFRTVIFDRPNIASYYHNQFPTIWHLQKYLMITDEK |
| NCBI Reference | ADIRLIYWALHSLLKHRGHFFNTTPMSQFKPGKLNLKDDMLALDDY |
| Sequence: | NDLEGLSFAVANSPEIEKVIKDRSMHKKEKIAELKKLIVNDVPDKDL |
| WP_133478044.1 | AKRNNKIITQIVNAIMGNSFHLNFIFDMDLDKLTSKAWSFKLDDPEL |
| Wild type | DTKFDAISGSMTDNQIGIFETLQKIYSAISLLDILNGSSNVVDAKNAL |
| | YDKHKRDLNLYFKFLNTLPDEIAKTLKAGYTLYIGNRKKDLLAARK |
| | LLKVNVAKNFSQDDFYKLINKELKSIDKQGLQTRFSEKVGELVAQN |
| | NFLPVQRSSDNVFIPYQLNAITFNKILENQGKYYDFLVKPNPAKKDR |
| | KNAPYELSQLMQFTIPYYVGPLVTPEEQVKSGIPKTSRFAWMVRKD |
| | NGAITPWNFYDKVDIEATADKFIKRSIAKDSYLLSELVLPKHSLLYE |
| | KYEVFNELSNVSLDGKKLSGGVKQILFNEVFKKTNKVNTSRILKAL |
| | AKHNIPGSKITGLSNPEEFTSSLQTYNAWKKYFPNQIDNFAYQQDLE |
| | KMIEWSTVFEDHKILAKKLDEIEWLDDDQKKFVANTRLRGWGRLS |
| | KRLLTGLKDNYGKSIMQRLETTKANFQQIVYKPEFREQIDKISQAAA |
| | KNQSLEDILANSYTSPSNRKAIRKTMSVVDEYIKLNHGKEPDKIFLM |
| | FQRSEQEKGKQTEARSKQLNRILSQLKADKSANKLFSKQLADEFSN |
| | AIKKSKYKLNDKQYFYFQQLGRDALTGEVIDYDELYKYTVLHIIPRS |
| | KLTDDSQNNKVLTKYKIVDGSVALKFGNSYSDALGMPIKAFWTEL |
| | NRLKLIPKGKLLNLTTDFSTLNKYQRDGYIARQLVETQQIVKLLATI |
| | MQSRFKHTKIIEVRNSQVANIRYQFDYFRIKNLNEYYRGFDAYLAA |
| | VVGTYLYKVYPKARRLFVYGQYLKPKKTNQENQDMHLDSEKKSQ |
| | GFNFLWNLLYGKQDQIFVNGTDVIAFNRKDLITKMNTVYNYKSQKI |
| | SLAIDYHNGAMFKATLFPRNDRDTAKTRKLIPKKKDYDTDIYGGYT |
| | SNVDGYMLLAEIIKRDGNKQYGFYGVPSRLVSELDTLKKTRYTEYE |
| | EKLKEIIKPELGVDLKKIKKIKILKNKVPFNQVIIDKGSKFFITSTSYR |
| | WNYRQLILSAESQQTLMDLVVDPDFSNHKARKDARKNADERLIKV |
| | YEEILYQVKNYMPMFVELHRCYEKLVDAQKTFKSLKISDKAMVLN |
| | QILILLHSNATSPVLEKLGYHTRFTLGKKHNLISENAVLVTQSITGLK |
| | ENHVSIKQML (SEQ ID NO: 15) |
| PdCas9 | MTNEKYSIGLDIGTSSIGFAVVNDNNRVIRVKGKNAIGVRLFDEGKA |
| Pedicoccus | AADRRSFRTTRRSFRTTRRRLSRRRWRLKLLREIFDAYITPVDEAFFI |
| damnosus | RLKESNLSPKDSKKQYSGDILFNDRSDKDFYEKYPTIYHLRNALMTE |
| NCBI Reference | HRKFDVREIYLAIHHIMKFRGHFLNATPANNFKVGRLNLEEKFEELN |
| Sequence: | DIYQRVFPDESIEFRTDNLEQIKEVLLDNKRSRADRQRTLVSDIYQSS |
| WP_062913273.1 | EDKDIEKRNKAVATEILKASLGNKAKLNVITNVEVDKEAAKEWSIT |
| Wild type | FDSESIDDDLAKIEGQMTDDGHEIIEVLRSLYSGITLSAIVPENHTLSQ |
| | SMVAKYDLHKDHLKLFKKLINGMTDTKKAKNLRAAYDGYIDGVK |
| | GKVLPQEDFYKQVQVNLDDSAEANEIQTYIDQDIFMPKQRTKANGS |
| | IPHQLQQQELDQIIENQKAYYPWLAELNPNPDKKRQQLAKYKLDEL |
| | VTFRVPYYVGPMITAKDQKNQSGAEFAWMIRKEPGNITPWNFDQK |
| | VDRMATANQFIKRMTTTDTYLLGEDVLPAQSLLYQKFEVLNELNKI |
| | RIDHKPISIEQKQQIFNDLFKQFKNVTIKHLQDYLVSQGQYSKRPLIE |
| | GLADEKRFNSSLSTYSDLCGIFGAKLVEENDRQEDLEKIIEWSTIFED |
| | KKIYRAKLNDLTWLTDDQKEKLATKRYQGWGRLSRKLLVGLKNSE |
| | HRNIMDILWITNENFMQIQAEPDFAKLVTDANKGMLEKTDSQDVIN |
| | DLYTSPQNKKAIRQILLVVHDIQNAMHGQAPAKIHVEFARGEERNP |
| | RRSVQRQRQVEAAYEKVSNELVSAKVRQEFKEAINNKRDFKDRLFL |
| | YFMQGGIDIYTGKQLNIDQLSSYQIDHILPQAFVKDDSLTNRVLTNE |
| | NQVKADSVPIDIFGKKMLSVWGRMKDQGLISKGKYRNLTMNPENIS |
| | AHTENGFINRQLVETRQVIKLAVNILADEYGDSTQIISVKADLSHQM |
| | REDFELLKNRDVNDYHHAFDAYLAAFIGNYLLKRYPKLESYFVYG |
| | DFKKFTQKETKMRRFNFIYDLKHCDQVVNKETGEILWTKDEDIKYI |
| | RHLFAYKKILVSHEVREKRGALYNQTIYKAKDDKGSGQESKKLIRIK |
| | DDKETKIYGGYSGKSLAYMTIVQITKKNKVSYRVIGIPTLALARLNK |
| | LENDSTENNGELYKIIKPQFTHYKVDKKNGEIIETTDDFKIVVSKVRF |
| | QQLIDDAGQFFMLASDTYKNNAQQLVISNNALKAINNTNITDCPRD |
| | DLERLDNLRLDSAFDEIVKKMDKYFSAYDANNFREKIRNSNLIFYQL |
| | PVEDQWENNKITELGKRTVLTRILQGLHANATTTDMSIFKIKTPFGQ |
| | LRQRSGISLSENAQLIYQSPTGLFERRVQLNKIK (SEQ ID NO: 16) |
| FnCas9 | MKKQKFSDYYLGFDIGTNSVGWCVTDLDYNVLRFNKKDMWGSRL |
| Fusobaterium | FEEAKTAAERRVQRNSRRRLKRRKWRLNLLEEIFSNEILKIDSNFFRR |
| nucleatum | LKESSLWLEDKSSKEKFTLFNDDNYKDYDFYKQYPTIFHLRNELIKN |
| NCBI Reference | PEKKDIRLVYLAIHSIFKSRGHFLFEGQNLKEIKNFETLYNNLIAFLED |
| Sequence: | NGINKIIDKNNIEKLEKIVCDSKKGLKDKEKEFKEIFNSDKQLVAIFK |
| WP_060798984.1 | LSVGSSVSLNDLFDTDEYKKGEVEKEKISFREQIYEDDKPIYYSILGE |
| | KIELLDIAKTFYDFMVLNNILADSQYISEAKVKLYEEHKKDLKNLKY |
| | IIRKYNKGNYDKLFKDKNENNYSAYIGLNKEKSKKEVIEKSRLKIDD |
| | LIKNIKGYLPKVEEIEEKDKAIFNKILNKIELKTILPKQRISDNGTLPY |
| | QIHEAELEKILENQSKYYDFLNYEENGIITKDKLLMTFKFRIPYYVGP |
| | LNSYHKDKGGNSWIVRKEEGKILPWNFEQKVDIEKSAEEFIKRMTN |
| | KCTYLNGEDVIPKDTFLYSEYVILNELNKVQVNDEFLNEENKRKIID |
| | ELFKENKKVSEKKFKEYLLVKQIVDGTIELKGVKDSFNSNYISYIRFK |
| | DIFGEKLNLDIYKEISEKSILWKCLYGDDKKIFEKKIKNEYGDILTKD |
| | EIKKINTFKFNNWGRLSEKLLTGIEFINLETGECYSSVMDALRRTNY |
| | NLMELLSSKFTLQESINNENKEMNEASYRDLIEESYVSPSLKRAIFQT |
| | LKIYEEIRKITGRVPKKVFIEMARGGDESMKNKKIPARQEQLKKLYD |
| | SCGNDIANFSIDIKEMKNSLISYDNNSLRQKKLYLYYLQFGKCMYTG |
| | REIDLDRLLQNNDTYDIDHIYPRSKVIKDDSFDNLVLVLKNENAEKS |
| | NEYPVKKEIQEKMKSFWRFLKEKNFISDEKYKRLTGKDDFELRGFM |
| | ARQLVNVRQTTKEVGKILQQIEPEIKIVYSKAEIASSFREMFDFIKVR |
| | ELNDTHHAKDAYLNIVAGNVYNTKFTEKPYRYLQEIKENYDVKKIY |
| | NYDIKNAWDKENSLEIVKKNMEKNTVNITRFIKEKKGQLFDLNPIK |
| | KGETSNEIISIKPKVYNGKDDKLNEKYGYYKSLNPAYFLYVEHKEK |
| | NKRIKSFERVNLVDVNNIKDEKSLVKYLIENKKLVEPRVIKKVYKRQ |
| | VILINDYPYSIVTLDSNKLMDFENLKPLFLENKYEKILKNVIKFLEDN |
| | QGKSEENYKFIYLKKKDRYEKNETLESVKDRYNLEFNEMYDKFLEK |
| | LDSKDYKNYMNNKKYQELLDVKEKFIKLNLFDKAFTLKSFLDLFNR |
| | KTMADFSKVGLTKYLGKIQKISSNVLSKNELYLLEESVTGLFVKKIK |
| | L (SEQ ID NO: 17) |
| EcCas9 | RRKQRIQILQELLGEEVLKTDPGFFHRMKESRYVVEDKRTLDGKQV |
| Enterococcus | ELPYALFVDKDYTDKEYYKQFPTINHLIVYLMTTSDTPDIRLVYLAL |
| cecorum | HYYMKNRGNFLHSGDINNVKDINDILEQLDNVLETFLDGWNLKLKS |
| NCBI Reference | YVEDIKNIYNRDLGRGERKKAFVNTLGAKTKAEKAFCSLISGGSTNL |
| Sequence: | AELFDDSSLKEIETPKIEFASSSLEDKIDGIQEALEDRFAVIEAAKRLY |
| WP_047338501.1 | DWKTLTDILGDSSSLAEARVNSYQMHHEQLLELKSLVKEYLDRKVF |
| Wild type | QEVFVSLNVANNYPAYIGHTKINGKKKELEVKRTKRNDFYSYVKK |
| | QVIEPIKKKVSDEAVLTKLSEIESLIEVDKYLPLQVNSDNGVIPYQVK |
| | LNELTRIFDNLENRIPVLRENRDKIIKTFKFRIPYYVGSLNGVVKNGK |
| | CTNWMVRKEEGKIYPWNFEDKVDLEASAEQFIRRMTNKCTYLVNE |
| | DVLPKYSLLYSKYLVLSELNNLRIDGRPLDVKIKQDIYENVFKKNRK |
| | VTLKKIKKYLLKEGIITDDDELSGLADDVKSSLTAYRDFKEKLGHLD |
| | LSEAQMENIILNITLFGDDKKLLKKRLAALYPFIDDKSLNRIATLNYR |
| | DWGRLSERFLSGITSVDQETGELRTIIQCMYETQANLMQLLAEPYHF |
| | VEAIEKENPKVDLESISYRIVNDLYVSPAVKRQIWQTLLVIKDIKQV |
| | MKHDPERIFIEMAREKQESKKTKSRKQVLSEVYKKAKEYEHLFEKL |
| | NSLTEEQLRSKKIYLYFTQLGKCMYSGEPIDFENLVSANSNYDIDHIY |
| | PQSKTIDDSFNNIVLVKKSLNAYKSNHYPIDKNIRDNEKVKTLWNTL |
| | VSKGLITKEKYERLIRSTPFSDEELAGFIARQLVETRQSTKAVAEILSN |
| | WFPESEIVYSKAKNVSNFRQDFEILKVRELNDCHHAHDAYLNIVVG |
| | NAYHTKFTNSPYRFIKNKANQEYNLRKLLQKVNKIESNGVVAWVG |
| | QSENNPGTIATVKKVIRRNTVLISRMVKEVDGQLFDLTLMKKGKGQ |
| | VPIKSSDERLTDISKYGGYNKATGAYFTFVKSKKRGKVVRSFEYVPL |
| | HLSKQFENNNELLKEYIEKDRGLTDVEILIPKVLINSLFRYNGSLVRIT |
| | GRGDTRLLLVHEQPLYVSNSFVQQLKSVSSYKLKKSENDNAKLTKT |
| | ATEKLSNIDELYDGLLRKLDLPIYSYWFSSIKEYLVESRTKYIKLSIEE |
| | KALVIFEILHLFQSDAQVPNLKILGLSTKPSRIRIQKNLKDTDKMSIIH |
| | QSPSGIFEHEIELTSL (SEQ ID NO: 18) |
| AhCas9 | MQNGFLGITVSSEQVGWAVTNPKYELERASRKDLWGVRLFDKAET |
| Anaerostipes | AEDRRMFRTNRRLNQRKKNRIHYLRDIFHEEVNQKDPNFFQQLDES |
| hadrus | NFCEDDRTVEFNFDTNLYKNQFPTVYHLRKYLMETKDKPDIRLVYL |
| NCBI Reference | AFSKFMKNRGHFLYKGNLGEVMDFENSMKGFCESLEKFNIDFPTLS |
| Sequence: | DEQVKEVRDILCDHKIAKTVKKKNIITITKVKSKTAKAWIGLFCGCS |
| WP_044924278.1 | VPVKVLFQDIDEEIVTDPEKISFEDASYDDYIANIEKGVGIYYEAIVSA |
| Wild type | KMLFDWSILNEILGDHQLLSDAMIAEYNKHHDDLKRLQKIIKGTGS |
| | RELYQDIFINDVSGNYVCYVGHAKTMSSADQKQFYTFLKNRLKNV |
| | NGISSEDAEWIDTEIKNGTLLPKQTKRDNSVIPHQLQLREFELILDNM |
| | QEMYPFLKENREKLLKIFNFVIPYYVGPLKGVVRKGESTNWMVPKK |
| | DGVIHPWNFDEMVDKEASAECFISRMTGNCSYLFNEKVLPKNSLLY |
| | ETFEVLNELNPLKINGEPISVELKQRIYEQLFLTGKKVTKKSLTKYLI |
| | KNGYDKDIELSGIDNEFHSNLKSHIDFEDYDNLSDEEVEQIILRITVFE |
| | DKQLLKDYLNREFVKLSEDERKQICSLSYKGWGNLSEMLLNGITVT |
| | DSNGVEVSVMDMLWNTNLNLMQILSKKYGYKAEIEHYNKEHEKTI |
| | YNREDLMDYLNIPPAQRRKVNQLITIVKSLKKTYGVPNKIFFKISREH |
| | QDDPKRTSSRKEQLKYLYKSLKSEDEKHLMKELDELNDHELSNDK |
| | VYLYFLQKGRCIYSGKKLNLSRLRKSNYQNDIDYIYPLSAVNDRSM |
| | NNKVLTGIQENRADKYTYFPVDSEIQKKMKGFWMELVLQGFMTKE |
| | KYFRLSRENDFSKSELVSFIEREISDNQQSGRMIASVLQYYFPESKIVF |
| | VKEKLISSFKRDFHLISSYGHNHLQAAKDAYITIVVGNVYHTKFTMD |
| | PAIYFKNHKRKDYDLNRLFLENISRDGQIAWESGPYGSIQTVRKEYA |
| | QNHIAVTKRVVEVKGGLFKQMPLKKGHGEYPLKTNDPRFGNIAQY |
| | GGYTNVTGSYFVLVESMEKGKKRISLEYVPVYLHERLEDDPGHKLL |
| | KEYLVDHRKLNHPKILLAKVRKNSLLKIDGFYYRLNGRSGNALILT |
| | NAVELIMDDWQTKTANKISGYMKRRAIDKKARVYQNEFHIQELEQ |
| | LYDFYLDKLKNGVYKNRKNNQAELIHNEKEQFMELKTEDQCVLLT |
| | EIKKLFVCSPMQADLTLIGGSKHTGMIAMSSNVTKADFAVIAEDPLG |
| | LRNKVIYSHKGEK (SEQ ID NO: 19) |
| KvCas9 | MSQNNNKIYNIGLDIGDASVGWAVVDEHYNLLKRHGKHMWGSRL |
| Kandleria | FTQANTAVERRSSRSTRRRYNKRRERIRLLREIMEDMVLDVDPTFFI |
| vitulina | RLANVSFLDQEDKKDYLKENYHSNYNLFIDKDFNDKTYYDKYPTIY |
| NCBI Reference | HLRKHLCESKEKEDPRLIYLALHHIVKYRGNFLYEGQKFSMDVSNIE |
| Sequence: | DKMIDVLRQFNEINLFEYVEDRKKIDEVLNVLKEPLSKKHKAEKAF |
| WP_031589969.1 | ALFDTTKDNKAAYKELCAALAGNKFNVTKMLKEAELHDEDEKDIS |
| Wild type | FKFSDATFDDAFVEKQPLLGDCVEFIDLLHDIYSWVELQNILGSAHT |
| | SEPSISAAMIQRYEDHKNDLKLLKDVIRKYLPKKYFEVFRDEKSKKN |
| | NYCNYINHPSKTPVDEFYKYIKKLIEKIDDPDVKTILNKIELESFMLK |
| | QNSRTNGAVPYQMQLDELNKILENQSVYYSDLKDNEDKIRSILTFRI |
| | PYYFGPLNITKDRQFDWIIKKEGKENERILPWNANEIVDVDKTADEF |
| | IKRMRNFCTYFPDEPVMAKNSLTVSKYEVLNEINKLRINDHLIKRDM |
| | KDKMLHTLFMDHKSISANAMKKWLVKNQYFSNTDDIKIEGFQKEN |
| | ACSTSLTPWIDFTKIFGKINESNYDFIEKIIYDVTVFEDKKILRRRLKK |
| | EYDLDEEKIKKILKLKYSGWSRLSKKLLSGIKTKYKDSTRTPETVLE |
| | VMERTNMNLMQVINDEKLGFKKTIDDANSTSVSGKFSYAEVQELA |
| | GSPAIKRGIWQALLIVDEIKKIMKHEPAHVYIEFARNEDEKERKDSF |
| | VNQMLKLYKDYDFEDETEKEANKHLKGEDAKSKIRSERLKLYYTQ |
| | MGKCMYTGKSLDIDRLDTYQVDHIVPQSLLKDDSIDNKVLVLSSEN |
| | QRKLDDLVIPSSIRNKMYGFWEKLFNNKIISPKKFYSLIKTEFNEKDQ |
| | ERFINRQIVETRQITKHVAQIIDNHYENTKVVTVRADLSHQFRER YHI |
| | YKNRDINDFHHAHDAYIATILGTYIGHRFESLDAKYIYGEYKRIFRN |
| | QKNKGKEMKKNNDGFILNSMRNIYADKDTGEIVWDPNYIDRIKKCF |
| | YYKDCFVTKKLEENNGTFFNVTVLPNDTNSDKDNTLATVPVNKYR |
| | SNVNKYGGFSGVNSFIVAIKGKKKKGKKVIEVNKLTGIPLMYKNAD |
| | EEIKINYLKQAEDLEEVQIGKEILKNQLIEKDGGLYYIVAPTEIINAKQ |
| | LILNESQTKLVCEIYKAMKYKNYDNLDSEKIIDLYRLLINKMELYYP |
| | EYRKQLVKKFEDRYEQLKVISIEEKCNIIKQILATLHCNSSIGKIMYS |
| | DFKISTTIGRLNGRTISLDDISFIAESPTGMYSKKYKL (SEQ ID NO: |
| | 20) |
| EfCas9 | MRLFEEGHTAEDRRLKRTARRRISRRRNRLRYLQAFFEEAMTDLDE |
| Enterococcus | NFFARLQESFLVPEDKKWHRHPIFAKLEDEVAYHETYPTIYHLRKKL |
| faecalis | ADSSEQADLRLIYLALAHIVKYRGHFLIEGKLSTENTSVKDQFQQFM |
| NCBI Reference | VIYNQTFVNGESRLVSAPLPESVLIEEELTEKASRTKKSEKVLQQFPQ |
| Sequence: | EKANGLFGQFLKLMVGNKADFKKVFGLEEEAKITYASESYEEDLEG |
| WP_016631044.1 | ILAKVGDEYSDVFLAAKNVYDAVELSTILADSDKKSHAKLSSSMIV |
| Wild type | RFTEHQEDLKKFKRFIRENCPDEYDNLFKNEQKDGYAGYIAHAGKV |
| | SQLKFYQYVKKIIQDIAGAEYFLEKIAQENFLRKQRTFDNGVIPHQIH |
| | LAELQAIIHRQAAYYPFLKENQEKIEQLVTFRIPYYVGPLSKGDASTF |
| | AWLKRQSEEPIRPWNLQETVDLDQSATAFIERMTNFDTYLPSEKVLP |
| | KHSLLYEKFMVFNELTKISYTDDRGIKANFSGKEKEKIFDYLFKTRR |
| | KVKKKDIIQFYRNEYNTEIVTLSGLEEDQFNASFSTYQDLLKCGLTR |
| | AELDHPDNAEKLEDIIKILTIFEDRQRIRTQLSTFKGQFSAEVLKKLER |
| | KHYTGWGRLSKKLINGIYDKESGKTILDYLVKDDGVSKHYNRNFM |
| | QLINDSQLSFKNAIQKAQSSEHEETLSETVNELAGSPAIKKGIYQSLKI |
| | VDELVAIMGYAPKRIVVEMARENQTTSTGKRRSIQRLKIVEKAMAEI |
| | GSNLLKEQPTTNEQLRDTRLFLYYMQNGKDMYTGDELSLHRLSHY |
| | DIDHIIPQSFMKDDSLDNLVLVGSTENRGKSDDVPSKEVVKDMKAY |
| | WEKLYAAGLISQRKFQRLTKGEQGGLTLEDKAHFIQRQLVETRQIT |
| | KNVAGILDQRYNAKSKEKKVQIITLKASLTSQFRSIFGLYKVREVND |
| | YHHGQDAYLNCVVATTLLKVYPNLAPEFVYGEYPKFQTFKENKAT |
| | AKAIIYTNLLRFFTEDEPRFTKDGEILWSNSYLKTIKKELNYHQMNIV |
| | KKVEVQKGGFSKESIKPKGPSNKLIPVKNGLDPQKYGGFDSPVVAY |
| | TVLFTHEKGKKPLIKQEILGITIMEKTRFEQNPILFLEEKGFLRPRVLM |
| | KLPKYTLYEFPEGRRRLLASAKEAQKGNQMVLPEHLLTLLYHAKQ |
| | CLLPNQSESLAYVEQHQPEFQEILERVVDFAEVHTLAKSKVQQIVKL |
| | FEANQTADVKEIAASFIQLMQFNAMGAPSTFKFFQKDIERARYTSIK |
| | EIFDATIIYQSPTGLYETRRKVVD (SEQ ID NO: 21) |
| Staphylococcus | KRNYILGLDIGITSVGYGIIDYETRDVIDAGVRLFKEANVENNEGRRS |
| aureus Cas9 | KRGARRLKRRRRHRIQRVKKLLFDYNLLTDHSELSGINPYEARVKG |
| | LSQKLSEEEFSAALLHLAKRRGVHNVNEVEEDTGNELSTKEQISRNS |
| | KALEEKYVAELQLERLKKDGEVRGSINRFKTSDYVKEAKQLLKVQ |
| | KAYHQLDQSFIDTYIDLLETRRTYYEGPGEGSPFGWKDIKEWYEML |
| | MGHCTYFPEELRSVKYAYNADLYNALNDLNNLVITRDENEKLEYY |
| | EKFQIIENVFKQKKKPTLKQIAKEILVNEEDIKGYRVTSTGKPEFTNL |
| | KVYHDIKDITARKEIIENAELLDQIAKILTIYQSSEDIQEELTNLNSELT |
| | QEEIEQISNLKGYTGTHNLSLKAINLILDELWHTNDNQIAIFNRLKLV |
| | PKKVDLSQQKEIPTTLVDDFILSPVVKRSFIQSIKVINAIIKKYGLPNDI |
| | IIELAREKNSKDAQKMINEMQKRNRQTNERIEEIIRTTGKENAKYLIE |
| | KIKLHDMQEGKCLYSLEAIPLEDLLNNPFNYEVDHIIPRSVSFDNSFN |
| | NKVLVKQEENSKKGNRTPFQYLSSSDSKISYETFKKHILNLAKGKGR |
| | ISKTKKEYLLEERDINRFSVQKDFINRNLVDTRYATRGLMNLLRSYF |
| | RVNNLDVKVKSINGGFTSFLRRKWKFKKERNKGYKHHAEDALIIAN |
| | ADFIFKEWKKLDKAKKVMENQMFEEKQAESMPEIETEQEYKEIFITP |
| | HQIKHIKDFKDYKYSHRVDKKPNRELINDTLYSTRKDDKGNTLIVN |
| | NLNGLYDKDNDKLKKLINKSPEKLLMYHHDPQTYQKLKLIMEQYG |
| | DEKNPLYKYYEETGNYLTKYSKKDNGPVIKKIKYYGNKLNAHLDIT |
| | DDYPNSRNKVVKLSLKPYRFDVYLDNGVYKFVTVKNLDVIKKENY |
| | YEVNSKCYEEAKKLKKISNQAEFIASFYNNDLIKINGELYRVIGVNN |
| | DLLNRIEVNMIDITYREYLENMNDKRPPRIIKTIASKTQSIKKYSTDIL |
| | GNLYEVKSKKHPQIIKKG (SEQ ID NO: 22) |
| Geobacillus | MKYKIGLDIGITSIGWAVINLDIPRIEDLGVRIFDRAENPKTGESLALP |
| thermodenitrificans | RRLARSARRRLRRRKHRLERIRRLFVREGILTKEELNKLFEKKHEID |
| Cas9 | VWQLRVEALDRKLNNDELARILLHLAKRRGFRSNRKSERTNKENST |
| | MLKHIEENQSILSSYRTVAEMVVKDPKFSLHKRNKEDNYTNTVARD |
| | DLEREIKLIFAKQREYGNIVCTEAFEHEYISIWASQRPFASKDDIEKK |
| | VGFCTFEPKEKRAPKATYTFQSFTVWEHINKLRLVSPGGIRALTDDE |
| | RRLIYKQAFHKNKITFHDVRTLLNLPDDTRFKGLLYDRNTTLKENE |
| | KVRFLELGAYHKIRKAIDSVYGKGAAKSFRPIDFDTFGYALTMFKD |
| | DTDIRSYLRNEYEQNGKRMENLADKVYDEELIEELLNLSFSKFGHLS |
| | LKALRNILPYMEQGEVYSTACERAGYTFTGPKKKQKTVLLPNIPPIA |
| | NPVVMRALTQARKVVNAIIKKYGSPVSIHIELARELSQSFDERRKMQ |
| | KEQEGNRKKNETAIRQLVEYGLTLNPTGLDIVKFKLWSEQNGKCAY |
| | SLQPIEIERLLEPGYTEVDHVIPYSRSLDDSYTNKVLVLTKENREKGN |
| | RTPAEYLGLGSERWQQFETFVLTNKQFSKKKRDRLLRLHYDENEEN |
| | EFKNRNLNDTRYISRFLANFIREHLKFADSDDKQKVYTVNGRITAHL |
| | RSRWNFNKNREESNLHHAVDAAIVACTTPSDIARVTAFYQRREQNK |
| | ELSKKTDPQFPQPWPHFADELQARLSKNPKESIKALNLGNYDNEKL |
| | ESLQPVFVSRMPKRSITGAAHQETLRRYIGIDERSGKIQTVVKKKLSE |
| | IQLDKTGHFPMYGKESDPRTYEAIRQRLLEHNNDPKKAFQEPLYKP |
| | KKNGELGPIIRTIKIIDTTNQVIPLNDGKTVAYNSNIVRVDVFEKDGK |
| | YYCVPIYTIDMMKGILPNKAIEPNKPYSEWKEMTEDYTFRFSLYPND |
| | LIRIEFPREKTIKTAVGEEIKIKDLFAYYQTIDSSNGGLSLVSHDNNFS |
| | LRSIGSRTLKRFEKYQVDVLGNIYKVRGEKRVGVASSSHSKAGETIR |
| | PL (SEQ ID NO: 23) |
| ScCas9 | MEKKYSIGLDIGTNSVGWAVITDDYKVPSKKFKVLGNTNRKSIKKN |
| S. canis | LMGALLFDSGETAEATRLKRTARRRYTRRKNRIRYLQEIFANEMAK |
| 1375 AA | LDDSFFQRLEESFLVEEDKKNERHPIFGNLADEVAYHRNYPTIYHLR |
| 159.2 kDa | KKLADSPEKADLRLIYLALAHIIKFRGHFLIEGKLNAENSDVAKLFY |
| | QLIQTYNQLFEESPLDEIEVDAKGILSARLSKSKRLEKLIAVFPNEKK |
| | NGLFGNIIALALGLTPNFKSNFDLTEDAKLQLSKDTYDDDLDELLGQ |
| | IGDQYADLFSAAKNLSDAILLSDILRSNSEVTKAPLSASMVKRYDEH |
| | HQDLALLKTLVRQQFPEKYAEIFKDDTKNGYAGYVGIGIKHRKRTT |
| | KLATQEEFYKFIKPILEKMDGAEELLAKLNRDDLLRKQRTFDNGSIP |
| | HQIHLKELHAILRRQEEFYPFLKENREKIEKILTFRIPYYVGPLARGNS |
| | RFAWLTRKSEEAITPWNFEEVVDKGASAQSFIERMTNFDEQLPNKK |
| | VLPKHSLLYEYFTVYNELTKVKYVTERMRKPEFLSGEQKKAIVDLL |
| | FKTNRKVTVKQLKEDYFKKIECFDSVEIIGVEDRFNASLGTYHDLLK |
| | IIKDKDFLDNEENEDILEDIVLTLTLFEDREMIEERLKTYAHLFDDKV |
| | MKQLKRRHYTGWGRLSRKMINGIRDKQSGKTILDFLKSDGFSNRNF |
| | MQLIHDDSLTFKEEIEKAQVSGQGDSLHEQIADLAGSPAIKKGILQT |
| | VKIVDELVKVMGHKPENIVIEMARENQTTTKGLQQSRERKKRIEEGI |
| | KELESQILKENPVENTQLQNEKLYLYYLQNGRDMYVDQELDINRLS |
| | DYDVDHIVPQSFIKDDSIDNKVLTRSVENRGKSDNVPSEEVVKKMK |
| | NYWRQLLNAKLITQRKFDNLTKAERGGLSEADKAGFIKRQLVETRQ |
| | ITKHVARILDSRMNTKRDKNDKPIREVKVITLKSKLVSDFRKDFQLY |
| | KVRDINNYHHAHDAYLNAVVGTALIKKYPKLESEFVYGDYKVYDV |
| | RKMIAKSEQEIGKATAKRFFYSNIMNFFKTEVKLANGEIRKRPLIETN |
| | GETGEVVWNKEKDFATVRKVLAMPQVNIVKKTEVQTGGFSKESILS |
| | KRESAKLIPRKKGWDTRKYGGFGSPTVAYSILVVAKVEKGKAKKL |
| | KSVKVLVGITIMEKGSYEKDPIGFLEAKGYKDIKKELIFKLPKYSLFE |
| | LENGRRRMLASATELQKANELVLPQHLVRLLYYTQNISATTGSNNL |
| | GYIEQHREEFKEIFEKIIDFSEKYILKNKVNSNLKSSFDEQFAVSDSIL |
| | LSNSFVSLLKYTSFGASGGFTFLDLDVKQGRLRYQTVTEVLDATLIY |
| | QSITGLYETRTDLSQLGGD (SEQ ID NO: 24) |
| SpCas9-NRCH | MDKKYSIGLDIGTNSVGWAVITDEYKVPSKKFKVLGNTDRHSIKKN |
| | LIGALLFDSGETAEATRLKRTARRRYTRRKNRICYLQEIFSNEMAKV |
| | DDSFFHRLEESFLVEEDKKHERHPIFGNIVDEVAYHEKYPTIYHLRK |
| | KLVDSTDKADLRLIYLALAHMIKFRGHFLIEGDLNPDNSDVDKLFIQ |
| | LVQTYNQLFEENPINASGVDAKAILSARLSKSRKLENLIAQLPGEKK |
| | NGLFGNLIALSLGLTPNFKSNFDLAEDAKLQLSKDTYDDDLDNLLA |
| | QIGDQYADLFLAAKNLSDAILLSDILRVNTEITKAPLSASMVKRYDE |
| | HHQDLTLLKALVRQQLPEKYKEIFFDQSKNGYAGYIDGGASQEEFY |
| | KFIKPILEKMDGTEELLVKLKREDLLRKQRTFDNGIIPHQIHLGELHA |
| | ILRRQGDFYPFLKDNREKIEKILTFRIPYYVGPLARGNSRFAWMTRK |
| | SEETITPWNFEEVVDKGASAQSFIERMTNFDKNLPNEKVLPKHSLLY |
| | EYFTVYNELTKVKYVTEGMRKPAFLSGEQKKAIVDLLFKTNRKVTV |
| | KQLKEDYFKKIECFDSVEISGVEDRFNASLGTYHDLLKIIKDKDFLD |
| | NEENEDILEDIVLTLTLFEDREMIEERLKTYAHLFDDKVMKQLKRLR |
| | YTGWGRLSRKLINGIRDKQSGKTILDFLKSDGFANRNFMQLIHDDSL |
| | TFKEDIQKAQVSGQGDSLHEHIANLAGSPAIKKGILQTVKVVDELVK |
| | VMGGHKPENIVIEMARENQTTQKGQKNSRERMKRIEEGIKELGSQIL |
| | KEHPVENTQLQNEKLYLYYLQNGRDMYVDQELDINRLSDYDVDAI |
| | VPQSFLKDDSIDNKVLTRSDKNRGKSDNVPSEEVVKKMKNYWRQL |
| | LNAKLITQRKFDNLTKAERGGLSELDKAGFIKRQLVETRQITKHVAQ |
| | ILDSRMNTKYDENDKLIREVKVITLKSKLVSDFRKDFQFYKVREINN |
| | YHHAHDAYLNAVVGTALIKKYPKLESEFVYGDYKVYDVRKMIAKS |
| | EQEIGKATAKYFFYSNIMNFFKTEITLANGEIRKRPLIETNGETGEIV |
| | WDKGRDFATVRKVLSMPQVNIVKKTEVQTGGFSKESILPKGNSDKL |
| | IARKKDWDPKKYGGFNSPTVAYSVLVVAKVEKGKSKKLKSVKELL |
| | GITIMERSSFEKNPIDFLEAKGYKEVKKDLIIKLPKYSLFELENGRKR |
| | MLASAGVLQKGNELALPSKYVNFLYLASHYEKLKGSPEDNEQKQL |
| | FVEQHKHYLDEIIEQISEFSKRVILADANLDKVLSAYNKHRDKPIREQ |
| | AENIIHLFTLTNLGAPAAFKYFDTTINRKQYNTTKEVLDATLIRQSIT |
| | GLYETRIDLSQLGGD (SEQ ID NO: 133) |

Additional Cas9 proteins are known in the art and will be readily apparent to those of skill in the art.

In some aspects, the present disclosure provides prime editors comprising any of reverse transcriptase variants and/or any of the Cas9 variants described herein. In some embodiments, the present disclosure provides prime editors comprising any of the reverse transcriptase variants provided herein and a napDNAbp. In certain embodiments, the napDNAbp comprises a Cas9 protein (e.g., a Cas9 nickase). In some embodiments, the Cas9 protein comprises any of the Cas9 proteins of SEQ ID NOs: 2, 6, 8, 9, 12-24, or 133, or an amino acid sequence at least 80%, at least 85%, at least 90%, at least 95%, at least 96%, at least 97%, at least 98%, or at least 99% identical to any one of SEQ ID NOs: 2, 6, 8, 9, 12-24, or 133. In certain embodiments, the Cas9 protein comprises the amino acid sequence of SEQ ID NO: 133, or an amino acid sequence at least 80%, at least 85%, at least 90%, at least 95%, at least 96%, at least 97%, at least 98%, or at least 99% identical to SEQ ID NO: 133. In certain embodiments, the napDNAbp comprises one of the Cas9 variants disclosed herein. In some embodiments, the present disclosure provides prime editors comprising any of the Cas9 variants provided herein and a polymerase. In some embodiments, the polymerase is a reverse transcriptase. In certain embodiments, the reverse transcriptase is one of the reverse transcriptase variants provided herein.

In some embodiments, the reverse transcriptase variant used in a prime editor comprises various amino acid substitutions relative to the amino acid sequence of Escherichia coli Ec48 reverse transcriptase (SEQ ID NO: 7), which is provided below:

(SEQ ID NO: 7)
GRPYVTLNLNGMFMDKFKPYSKSNAPITTLEKLSKALSISVEELKAIAEL
SLDEKYTLKEIPKIDGSKRIVYSLHPKMRLLQSRINKRIFKELVVFPSFL
FGSVPSKNDVLNSNVKRDYVSCAKAHCGAKTVLKVDISNFFDNIHRDLVR
SVFEEILHIKDEALEYLVDICTKDDFVVQGALTSSYIATLCLFAVEGDVV
RRAQRKGLVYTRLVDDITVSSKISNYDFSQMQSHIERMLSEHDLPINKHK
TKIFHCSSEPIKVHGLRVDYDSPRLPSDEVKRIRASIHNLKLLAAKNNTK
TSVAYRKEFNRCMGRVNKLGRVGHEKYESFKKQLQAIKPMPSKRDVAVID
AAIKSLELSYSKGNQNKHWYKRKYDLTRYKMIILTRSESFKEKLECFKSR
LASLKPL

In some embodiments, the reverse transcriptase variant used in a prime editor comprises a sequence having at least 80%, at least 85%, at least 90%, at least 95%, at least 96%, at least 97%, at least 98%, or at least 99% sequence identity with SEQ ID NO: 7, wherein the reverse transcriptase comprises amino acid substitutions at positions 60, 87, 165, 243, 267, 279, 318, and 343 relative to SEQ ID NO: 7. In some embodiments, the amino acid substitution at position 60 is an E60X substitution, wherein X is any amino acid other than wild type. In certain embodiments, the amino acid substitution at position 60 is an E60K substitution. In some embodiments, the amino acid substitution at position 87 is a K87X substitution, wherein X is any amino acid other than wild type. In certain embodiments, the amino acid substitution at position 87 is a K87E substitution. In some embodiments, the amino acid substitution at position 165 is an E165X substitution, wherein X is any amino acid other than wild type. In certain embodiments, the amino acid substitution at position 165 is an E165D substitution. In some embodiments, the amino acid substitution at position 243 is a D243X substitution, wherein X is any amino acid other than wild type. In certain embodiments, the amino acid substitution at position 243 is a D243N substitution. In some embodiments, the amino acid substitution at position 267 is an R267X substitution, wherein X is any amino acid other than wild type. In certain embodiments, the amino acid substitution at position 267 is an R267I substitution. In some embodiments, the amino acid substitution at position 279 is an E279X substitution, wherein X is any amino acid other than wild type. In certain embodiments, the amino acid substitution at position 279 is an E279K substitution. In some embodiments, the amino acid substitution at position 318 is a K318X substitution, wherein X is any amino acid other than wild type. In certain embodiments, the amino acid substitution at position 318 is a K318E substitution. In some embodiments, the amino acid substitution at position 343 is a K343X substitution, wherein X is any amino acid other than wild type. In certain embodiments, the amino acid substitution at position 343 is a K343N substitution. In certain embodiments, the reverse transcriptase variant comprises the amino acid substitutions E60K, K87E, E165D, D243N, R267I, E279K, K318E, and K343N relative to SEQ ID NO: 7.

In certain embodiments, the reverse transcriptase variant comprises the amino acid sequence of SEQ ID NO: 50 (the RT domain of “PE6a”), or an amino acid sequence at least 80%, at least 85%, at least 90%, at least 95%, at least 96%, at least 97%, at least 98%, or at least 99% identical to the amino acid sequence of SEQ ID NO: 50:

(SEQ ID NO: 50)
GRPYVTLNLNGMFMDKFKPYSKSNAPITTLEKLSKALSISVEELKAIAEL
SLDEKYTLKKIPKIDGSKRIVYSLHPKMRLLQSRINERIFKELVVFPSFL
FGSVPSKNDVLNSNVKRDYVSCAKAHCGAKTVLKVDISNFFDNIHRDLVR
SVFEEILHIKDEALDYLVDICTKDDFVVQGALTSSYIATLCLFAVEGDVV
RRAQRKGLVYTRLVDDITVSSKISNYDFSQMQSHIERMLSEHNLPINKHK
TKIFHCSSEPIKVHGLIVDYDSPRLPSDKVKRIRASIHNLKLLAAKNNTK
TSVAYRKEFNRCMGRVNELGRVGHEKYESFKKQLQAIKPMPSNRDVAVID
AAIKSLELSYSKGNQNKHWYKRKYDLTRYKMIILTRSESFKEKLECFKSR
LASLKPL

In some embodiments, a PE6a prime editor comprises the amino acid sequence:

(SEQ ID NOs: 146, 154)
MKRTADGSEFESPKKKRKV[CAS9]SGGSSGGSKRTADGSEFESPKKKRK
VSGGSSGGSGRPYVTLNLNGMFMDKFKPYSKSNAPITTLEKLSKALSISV
EELKAIAELSLDEKYTLKKIPKIDGSKRIVYSLHPKMRLLQSRINERIFK
ELVVFPSFLFGSVPSKNDVLNSNVKRDYVSCAKAHCGAKTVLKVDISNFF
DNIHRDLVRSVFEEILHIKDEALDYLVDICTKDDFVVQGALTSSYIATLC
LFAVEGDVVRRAQRKGLVYTRLVDDITVSSKISNYDFSQMQSHIERMLSE
HNLPINKHKTKIFHCSSEPIKVHGLIVDYDSPRLPSDKVKRIRASIHNLK
LLAAKNNTKTSVAYRKEFNRCMGRVNELGRVGHEKYESFKKQLQAIKPMP
SNRDVAVIDAAIKSLELSYSKGNQNKHWYKRKYDLTRYKMIILTRSESFK
EKLECFKSRLASLKPLKRTADGSEFESPKKKRKVPAAKRVKLD,
wherein [CAS9] comprises any Cas9 protein (e.g.,
any of the Cas9 variants described herein).

The prime editors provided herein may, in some embodiments, comprise both a reverse transcriptase variant provided herein and a Cas9 variant provided herein, or reverse transcriptase variants and Cas9 variants at least 80%, at least 85%, at least 90%, at least 95%, at least 96%, at least 97%, at least 98%, or at least 99% identical to any of those provided herein. For example, the present disclosure contemplates prime editors comprising the reverse transcriptase variant of SEQ ID NO: 50 (PE6a) and the Cas9 variant of SEQ ID NO: 28 (PE6e), or a reverse transcriptase variant and Cas9 variant at least 80%, at least 85%, at least 90%, at least 95%, at least 96%, at least 97%, at least 98%, or at least 99% identical to SEQ ID NO: 50 and SEQ ID NO: 28. In some embodiments, a prime editor comprises the reverse transcriptase variant of SEQ ID NO: 50 (PE6a) and the Cas9 variant of SEQ ID NO: 48 (PE6f), or a reverse transcriptase variant and Cas9 variant at least 80%, at least 85%, at least 90%, at least 95%, at least 96%, at least 97%, at least 98%, or at least 99% identical to SEQ ID NO: 50 and SEQ ID NO: 48. In some embodiments, a prime editor comprises the reverse transcriptase variant of SEQ ID NO: 50 (PE6a) and the Cas9 variant of SEQ ID NO: 49 (PE6g), or a reverse transcriptase variant and Cas9 variant at least 80%, at least 85%, at least 90%, at least 95%, at least 96%, at least 97%, at least 98%, or at least 99% identical to SEQ ID NO: 50 and SEQ ID NO: 49. In some embodiments, a prime editor comprises the reverse transcriptase variant of SEQ ID NO: 25 (PE6b) and the Cas9 variant of SEQ ID NO: 28 (PE6e), or a reverse transcriptase variant and Cas9 variant at least 80%, at least 85%, at least 90%, at least 95%, at least 96%, at least 97%, at least 98%, or at least 99% identical to SEQ ID NO: 25 and SEQ ID NO: 28. In some embodiments, a prime editor comprises the reverse transcriptase variant of SEQ ID NO: 25 (PE6b) and the Cas9 variant of SEQ ID NO: 48 (PE6f), or a reverse transcriptase variant and Cas9 variant at least 80%, at least 85%, at least 90%, at least 95%, at least 96%, at least 97%, at least 98%, or at least 99% identical to SEQ ID NO: 25 and SEQ ID NO: 48. In some embodiments, a prime editor comprises the reverse transcriptase variant of SEQ ID NO: 25 (PE6b) and the Cas9 variant of SEQ ID NO: 49 (PE6g), or a reverse transcriptase variant and Cas9 variant at least 80%, at least 85%, at least 90%, at least 95%, at least 96%, at least 97%, at least 98%, or at least 99% identical to SEQ ID NO: 25 and SEQ ID NO: 49. In some embodiments, a prime editor comprises the reverse transcriptase variant of SEQ ID NO: 26 (PE6c) and the Cas9 variant of SEQ ID NO: 28 (PE6e), or a reverse transcriptase variant and Cas9 variant at least 80%, at least 85%, at least 90%, at least 95%, at least 96%, at least 97%, at least 98%, or at least 99% identical to SEQ ID NO: 26 and SEQ ID NO: 28. In some embodiments, a prime editor comprises the reverse transcriptase variant of SEQ ID NO: 26 (PE6c) and the Cas9 variant of SEQ ID NO: 48 (PE6f), or a reverse transcriptase variant and Cas9 variant at least 80%, at least 85%, at least 90%, at least 95%, at least 96%, at least 97%, at least 98%, or at least 99% identical to SEQ ID NO: 26 and SEQ ID NO: 48. In some embodiments, a prime editor comprises the reverse transcriptase variant of SEQ ID NO: 26 (PE6c) and the Cas9 variant of SEQ ID NO: 49 (PE6g), or a reverse transcriptase variant and Cas9 variant at least 80%, at least 85%, at least 90%, at least 95%, at least 96%, at least 97%, at least 98%, or at least 99% identical to SEQ ID NO: 26 and SEQ ID NO: 49. In some embodiments, a prime editor comprises the reverse transcriptase variant of SEQ ID NO: 27 (PE6d) and the Cas9 variant of SEQ ID NO: 28 (PE6e), or a reverse transcriptase variant and Cas9 variant at least 80%, at least 85%, at least 90%, at least 95%, at least 96%, at least 97%, at least 98%, or at least 99% identical to SEQ ID NO: 27 and SEQ ID NO: 28. In some embodiments, a prime editor comprises the reverse transcriptase variant of SEQ ID NO: 27 (PE6d) and the Cas9 variant of SEQ ID NO: 48 (PE6f), or a reverse transcriptase variant and Cas9 variant at least 80%, at least 85%, at least 90%, at least 95%, at least 96%, at least 97%, at least 98%, or at least 99% identical to SEQ ID NO: 27 and SEQ ID NO: 48. In some embodiments, a prime editor comprises the reverse transcriptase variant of SEQ ID NO: 27 (PE6d) and the Cas9 variant of SEQ ID NO: 49 (PE6g), or a reverse transcriptase variant and Cas9 variant at least 80%, at least 85%, at least 90%, at least 95%, at least 96%, at least 97%, at least 98%, or at least 99% identical to SEQ ID NO: 27 and SEQ ID NO: 49.

### Nuclear Localization Sequences (NLS)

In various embodiments, the prime editors described herein may comprise one or more nuclear localization sequences (NLS), which help promote translocation of a protein into the cell nucleus. Such sequences are well-known in the art and can include the following examples:

| DESCRIPTION | SEQUENCE | SEQ ID NO: |
|---|---|---|
| NLS OF SV40 LARGE | PKKKRKV | 94 |
| T-AG | | |
| NLS | MKRTADGSEFESPKKKRKV | 95 |
| NLS | MDSLLMNRRKFLYQFKNVRWAKG | 99 |
| | RRETYLC | |
| NLS OF | AVKRPAATKKAGQAKKKKLD | 100 |
| NUCLEOPLASMIN | | |
| NLS OF EGL-13 | MSRRRKANPTKLSENAKKLAKEV | 101 |
| | EN | |
| NLS OF C-MYC | PAAKRVKLD | 98 |
| NLS OF TUS-PROTEIN | KLKIKRPVK | 102 |
| NLS OF POLYOMA | VSRKRPRP | 103 |
| LARGE T-AG | | |
| NLS OF HEPATITIS D | EGAPPAKRAR | 104 |
| VIRUS ANTIGEN | | |
| NLS OF MURINE P53 | PPQPKKKPLDGE | 105 |
| NLS OF PE1 AND PE2 | SGGSKRTADGSEFEPKKKRKV | 96 |
| BIPARTITE SV40 NLS | KRTADGSEFESPKKKRKV | 97 |

The NLS examples above are non-limiting. The prime editors disclosed herein may comprise any known NLS sequence, including any of those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, each of which are incorporated herein by reference.

In various embodiments, the prime editors described herein further comprise one or more (and preferably at least two) nuclear localization sequences. In certain embodiments, the prime editors comprise at least two NLSs. In embodiments with at least two NLSs, the NLSs can be the same NLSs or they can be different NLSs. In some embodiments, one or more of the NLSs are bipartite NLSs (“bpNLS”). In certain embodiments, the prime editors comprise two bipartite NLSs. In some embodiments, the prime editors comprise more than two bipartite NLSs.

The location of the NLS fusion can be at the N-terminus, the C-terminus, or within a sequence of a prime editor (e.g., inserted between the encoded napDNAbp component (e.g., Cas9) and a polymerase domain (e.g., a reverse transcriptase).

The NLSs may be any known NLS sequence in the art. The NLSs may also be any future-discovered NLSs for nuclear localization. The NLSs also may be any naturally-occurring NLS, or any non-naturally occurring NLS (e.g., an NLS with one or more desired mutations).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT application PCT/EP2000/011690, filed Nov. 23, 2000, published as WO/2001/038547 on May 31, 2001, the contents of which are incorporated herein by reference. In some embodiments, an NLS comprises the amino acid sequence PKKKRKV (SEQ ID NO: 94), MDSLLMNRRKFLYQFKNVRWAKGRRETYLC (SEQ ID NO: 99), KRTADGSEFESPKKKRKV (SEQ ID NO: 97), or KRTADGSEFEPKKKRKV (SEQ ID NO: 106). In other embodiments, an NLS comprises the amino acid sequences NLSKRPAAIKKAGQAKKKK (SEQ ID NO: 107), PAAKRVKLD (SEQ ID NO: 98), RQRRNELKRSF (SEQ ID NO: 108), or NQSSNFGPMKGGNFGGRSSGPYGGGGQYFAKPRNQGGY (SEQ ID NO: 109).

In one aspect of the disclosure, a prime editor described herein may be modified with one or more nuclear localization sequences (NLS), preferably at least two NLSs. In certain embodiments, the prime editors are modified with two or more NLSs. The disclosure contemplates the use of any nuclear localization sequence known in the art at the time of the disclosure, or any nuclear localization sequence that is identified or otherwise made available in the state of the art after the time of the instant filing. A representative nuclear localization sequence is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization sequences often comprise proline residues. A variety of nuclear localization sequences have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated herein by reference. Translocation is currently thought to involve nuclear pore proteins.

Most NLSs can be classified in three general groups: (i) a monopartite NLS exemplified by the SV40 large T antigen NLS (PKKKRKV (SEQ ID NO: 94)); (ii) a bipartite motif consisting of two basic domains separated by a variable number of spacer amino acids and exemplified by the Xenopus nucleoplasmin NLS (KRXXXXXXXXXXKKKL (SEQ ID NO: 110)); and (iii) noncanonical sequences such as M9 of the hnRNP A1 protein, the influenza virus nucleoprotein NLS, and the yeast Gal4 protein NLS (Robbins, J. et al., Cell 1991, 64(3), 615-623).

Nuclear localization sequences appear at various points in the amino acid sequences of proteins. NLS have been identified at the N-terminus, the C-terminus, and in the central region of proteins. Thus, the disclosure provides prime editors that may be modified with one or more NLSs at the C-terminus and/or the N-terminus, as well as at internal regions of the prime editor. The residues of a longer sequence that do not function as component NLS residues should be selected so as not to interfere, for example, tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a prime editor to include one or more NLSs. In one aspect, the prime editors may be engineered to express a prime editor that is translationally fused at its N-terminus or its C-terminus (or both) to one or more NLSs, i.e., to form a prime editor-NLS fusion construct. In other embodiments, a prime editor-encoding nucleotide sequence may be genetically modified to incorporate a reading frame that encodes one or more NLSs in an internal region of the encoded prime editor. In addition, the NLSs may include various amino acid linkers or spacer regions encoded between the prime editor and the N-terminally, C-terminally, or internally attached NLS amino acid sequence, e.g., and in the central region of proteins. Thus, the present disclosure also provides for nucleotide constructs, vectors, and host cells for expressing fusion proteins that comprise a prime editor and one or more NLSs, among other components.

The prime editors described herein may also comprise nuclear localization sequences that are linked to a prime editor through one or more linkers, e.g., a polymeric, amino acid, nucleic acid, polysaccharide, chemical, or nucleic acid linker element. The linkers within the contemplated scope of the disclosure are not intended to have any limitations and can be any suitable type of molecule (e.g., polymer, amino acid, polysaccharide, nucleic acid, lipid, or any synthetic chemical linker domain) and can be joined to the prime editor by any suitable strategy that effectuates forming a bond (e.g., covalent linkage, hydrogen bonding) between the prime editor and the one or more NLSs.

In some embodiments, the prime editors provided herein comprise an NLS comprising the amino acid sequence of SEQ ID NO: 95, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 95. In some embodiments, the prime editors provided herein comprise an NLS comprising the amino acid sequence of SEQ ID NO: 97, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 97. In some embodiments, the prime editors provided herein comprise an NLS comprising the amino acid sequence of SEQ ID NO: 98, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 98. In certain embodiments, the prime editors provided herein comprise a first NLS comprising the amino acid sequence of SEQ ID NO: 95, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 95, a second NLS comprising the amino acid sequence of SEQ ID NO: 97, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 97, and a third NLS comprising the amino acid sequence of SEQ ID NO: 98, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 98.

### Linkers

In various embodiments, the napDNAbp and the reverse transcriptase of the prime editors provided herein may be provided in trans or otherwise not fused to one another. In other embodiments, the prime editors provided herein comprise a napDNAbp and a reverse transcriptase fused to one another, for example, via one or more linkers. As defined above, the term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and a polymerase (e.g., a reverse transcriptase). In some embodiments, a linker joins a Cas9 protein and a reverse transcriptase (e.g., any of the Cas9 variants provided herein and/or any of the reverse transcriptase variants provided herein). Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The linker may be as simple as a covalent bond, or it may be a polymeric linker many atoms in length. In certain embodiments, the linker is a polypeptide, or amino acid-based. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched, aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)ₙ (SEQ ID NO: 84), (G)ₙ (SEQ ID NO: 85), (EAAAK)ₙ (SEQ ID NO: 86), (GGS)ₙ (SEQ ID NO: 87), (SGGS)ₙ (SEQ ID NO: 81), (XP)ₙ (SEQ ID NO: 88), or any combination thereof, wherein n is independently an integer between 1 and 30, and wherein X is any amino acid. In some embodiments, the linker comprises the amino acid sequence (GGS)ₙ (SEQ ID NO: 87), wherein n is 1, 3, or 7. In some embodiments, the linker comprises the amino acid sequence SGSETPGTSESATPES (SEQ ID NO: 89). In some embodiments, the linker comprises the amino acid sequence SGGSSGGSSGSETPGTSESATPESSGGSSGGS (SEQ ID NO: 90). In some embodiments, the linker comprises the amino acid sequence SGGSGGSGGS (SEQ ID NO: 91). In some embodiments, the linker comprises the amino acid sequence SGGS (SEQ ID NO: 82). In other embodiments, the linker comprises the amino acid sequence SGGSSGGSSGSETPGTSESATPESAGSYPYDVPDYAGSAAPAAKKKKLDGSGSGGSS GGS (SEQ ID NO: 83, 60AA). In some embodiments, the linker comprises the amino acid sequence GGS, GGSGGS (SEQ ID NO: 92), GGSGGSGGS (SEQ ID NO: 93), SGGSSGGSSGSETPGTSESATPESSGGSSGGSS (SEQ ID NO: 80), SGSETPGTSESATPES (SEQ ID NO: 89), or SGGSSGGSSGSETPGTSESATPESAGSYPYDVPDYAGSAAPAAKKKKLDGSGSGGSS GGS (SEQ ID NO: 83).

In certain embodiments, linkers may be used to link any of the peptides or peptide domains or moieties of the invention (e.g., a napDNAbp linked or fused to a reverse transcriptase domain, and/or a napDNAbp linked to one or more NLS). Any of the domains of the prime editors described herein may also be connected to one another through any of the presently described linkers.

### PegRNAs

The prime editing systems and methods described herein contemplate the use of any suitable pegRNAs, e.g., to introduce recombinase recognition sites into a target DNA sequence, such as a genome, using prime editing.

### PEgRNA Architecture

In some embodiments, an extended guide RNA, or pegRNA, used in the prime editing systems and methods disclosed herein includes a spacer sequence (e.g., a ~20 nt spacer sequence) and a gRNA core region, which binds with the napDNAbp. In some embodiments, the pegRNA includes an extended RNA segment, i.e., an extension arm, at the 5′ end, i.e., a 5′ extension. In some embodiments, the 5′ extension includes a DNA synthesis template sequence, a primer binding site, and an optional 5-20 nucleotide linker sequence. The RT primer binding site hybridizes to the free 3′ end that is formed after a nick is formed in the non-target strand of the R-loop, thereby priming reverse transcriptase for DNA polymerization in the 5′-3′ direction.

In another embodiment, an extended guide RNA (i.e., a pegRNA) used in the prime editing systems and methods provided herein includes a spacer sequence (e.g., a ~20 nt spacer sequence) and a gRNA core, which binds with the napDNAbp. In some embodiments, the pegRNA includes an extended RNA segment, i.e., an extension arm, at the 3′ end, i.e., a 3′ extension. In some embodiments, the 3′ extension includes a DNA synthesis template sequence, and a reverse transcription primer binding site. The RT primer binding site hybridizes to the free 3′ end that is formed after a nick is formed in the non-target strand of the R-loop, thereby priming reverse transcriptase for DNA polymerization in the 5′-3′ direction.

In another embodiment, an extended guide RNA (i.e., a pegRNA) used in the prime editing systems and methods provided herein includes a spacer sequence (e.g., a ~20 nt spacer sequence) and a gRNA core, which binds with the napDNAbp. In some embodiments, the pegRNA includes an extended RNA segment, i.e., an extension arm, at an intermolecular position within the gRNA core, i.e., an intramolecular extension. In some embodiments, the intramolecular extension includes a DNA synthesis template sequence, and a reverse transcription primer binding site. The RT primer binding site hybridizes to the free 3′ end that is formed after a nick is formed in the non-target strand of the R-loop, thereby priming reverse transcriptase for DNA polymerization in the 5′-3′ direction.

In one embodiment, the position of the intermolecular RNA extension is not in the spacer sequence of the guide RNA. In another embodiment, the position of the intermolecular RNA extension is in the gRNA core. In still another embodiment, the position of the intermolecular RNA extension is anywhere within the guide RNA molecule except within the spacer sequence, or at a position which disrupts the spacer sequence. In one embodiment, the intermolecular RNA extension is inserted downstream from the 3′ end of the spacer sequence. In another embodiment, the intermolecular RNA extension is inserted at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, or at least 25 nucleotides downstream of the 3′ end of the spacer sequence.

In other embodiments, the intermolecular RNA extension is inserted into the gRNA core, which refers to the portion of a traditional guide RNA corresponding or comprising the tracrRNA, which binds and/or interacts with the napDNAbp, e.g., a Cas9 protein or equivalent thereof (i.e., a different napDNAbp). Preferably the insertion of the intermolecular RNA extension does not disrupt or minimally disrupts the interaction between the tracrRNA portion and the napDNAbp.

The length of the RNA extension (which includes at least the RT template and primer binding site) can be any useful length. In various embodiments, the RNA extension is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The RT template sequence can also be any suitable length. For example, the RT template sequence can be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In still other embodiments, the reverse transcription primer binding site sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In other embodiments, the optional linker or spacer sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The RT template sequence, in certain embodiments, encodes a single-stranded DNA molecule which is homologous to the non-target strand (and thus, complementary to the corresponding site of the target strand) but includes one or more nucleotide changes, e.g., for introducing a recombinase recognition sequence into a target DNA molecule. The one or more nucleotide changes may include one or more single-base nucleotide changes, one or more deletions, and/or one or more insertions.

The synthesized single-stranded DNA product of the RT template sequence is homologous to the non-target strand except that it contains one or more nucleotide changes. The single-stranded DNA product of the RT template sequence hybridizes in equilibrium with the complementary target strand sequence, thereby displacing the homologous endogenous target strand sequence. The displaced endogenous strand may be referred to in some embodiments as a 5′ endogenous DNA flap species. This 5′ endogenous DNA flap species can be removed by a 5′ flap endonuclease (e.g., FEN1) and the single-stranded DNA product, now hybridized to the endogenous target strand, may be ligated, thereby creating a mismatch between the endogenous sequence and the newly synthesized strand. The mismatch may be resolved by the cell's innate DNA repair and/or replication processes.

In various embodiments, the nucleotide sequence of the RT template sequence corresponds to the nucleotide sequence of the non-target strand that becomes displaced as the 5′ flap species and that overlaps with the site to be edited.

In various embodiments of the extended guide RNAs, the DNA synthesis template sequence may encode a single-strand DNA flap that is complementary to an endogenous DNA sequence adjacent to a nick site, wherein the single-strand DNA flap comprises a desired nucleotide change. The single-stranded DNA flap may displace an endogenous single-strand DNA at the nick site. The displaced endogenous single-strand DNA at the nick site can have a 5′ end and form an endogenous flap, which can be excised by the cell. In various embodiments, excision of the 5′ end endogenous flap can help drive product formation since removing the 5′ end endogenous flap encourages hybridization of the single-strand 3′ DNA flap to the corresponding complementary DNA strand, and the incorporation or assimilation of the desired nucleotide change carried by the single-strand 3′ DNA flap into the target DNA.

The terms “cleavage site,” “nick site,” and “cut site” as used interchangeably herein in the context of prime editing, refer to a specific position in between two nucleotides or two base pairs in the double-stranded target DNA sequence. In some embodiments, the position of a nick site is determined relative to the position of a specific PAM sequence. In some embodiments, the nick site is the particular position where a nick will occur when the double stranded target DNA is contacted with a napDNAbp, e.g., a nickase such as a Cas nickase, that recognizes a specific PAM sequence. For each PEgRNA described herein, a nick site (e.g., the “first nick site” when referred to in the context of PE3, PE5 and similar approaches), is characteristic of the particular napDNAbp to which the gRNA core of the PEgRNA associates with, and is characteristic of the particular PAM required for recognition and function of the napDNAbp. For example, for a PEgRNA that comprises a gRNA core that associates with a SpCas9, the nick site in the phosphodiester bond between bases three (“−3” position relative to the position 1 of the PAM sequence) and four (“−4” position relative to position 1 of the PAM sequence).

In some embodiments, a nick site is in a target strand of the double-stranded target DNA sequence. In some embodiments, a nick site is in a non-target strand of the double-stranded target DNA sequence. In some embodiments, the nick site is in a protospacer sequence. In some embodiments, the nick site is adjacent to a protospacer sequence. In some embodiments, a nick site is downstream of a region, e.g., on a non-target strand, that is complementary to a primer binding site of a PEgRNA. In some embodiments, a nick site is downstream of a region, e.g., on a non-target strand, that binds to a primer binding site of a PEgRNA. In some embodiments, a nick site is immediately downstream of a region, e.g., on a non-target strand, that is complementary to a primer binding site of a PEgRNA. In some embodiments, the nick site is upstream of a specific PAM sequence on the non-target strand of the double stranded target DNA, wherein the PAM sequence is specific for recognition by a napDNAbp that associates with the gRNA core of a PEgRNA. In some embodiments, the nick site is downstream of a specific PAM sequence on the non-target strand of the double stranded target DNA, wherein the PAM sequence is specific for recognition by a napDNAbp that associates with the gRNA core of a PEgRNA. In some embodiments, the nick site is 3 nucleotides upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 nucleotides upstream of the PAM sequence, and the PAM sequence is recognized by a Cas9 nickase, wherein the Cas9 nickase comprises a nuclease active HNH domain and a nuclease inactive RuvC domain. In some embodiments, the nick site is 2 base pairs upstream of the PAM sequence, and the PAM sequence is recognized by a S. thermophilus Cas9 nickase.

In various embodiments of the extended guide RNAs, the cellular repair of the single-strand DNA flap results in installation of the desired nucleotide change, thereby forming a desired product.

In still other embodiments, the desired nucleotide change is installed in an editing window that is between about −5 to +5 of the nick site, or between about −10 to +10 of the nick site, or between about −20 to +20 of the nick site, or between about −30 to +30 of the nick site, or between about −40 to +40 of the nick site, or between about −50 to +50 of the nick site, or between about −60 to +60 of the nick site, or between about −70 to +70 of the nick site, or between about −80 to +80 of the nick site, or between about −90 to +90 of the nick site, or between about −100 to +100 of the nick site, or between about −200 to +200 of the nick site.

In other embodiments, the desired nucleotide change is installed in an editing window that is between about +1 to +2 from the nick site, or about +1 to +3, +1 to +4, +1 to +5, +1 to +6, +1 to +7, +1 to +8, +1 to +9, +1 to +10, +1 to +11, +1 to +12, +1 to +13, +1 to +14, +1 to +15, +1 to +16, +1 to +17, +1 to +18, +1 to +19, +1 to +20, +1 to +21, +1 to +22, +1 to +23, +1 to +24, +1 to +25, +1 to +26, +1 to +27, +1 to +28, +1 to +29, +1 to +30, +1 to +31, +1 to +32, +1 to +33, +1 to +34, +1 to +35, +1 to +36, +1 to +37, +1 to +38, +1 to +39, +1 to +40, +1 to +41, +1 to +42, +1 to +43, +1 to +44, +1 to +45, +1 to +46, +1 to +47, +1 to +48, +1 to +49, +1 to +50, +1 to +51, +1 to +52, +1 to +53, +1 to +54, +1 to +55, +1 to +56, +1 to +57, +1 to +58, +1 to +59, +1 to +60, +1 to +61, +1 to +62, +1 to +63, +1 to +64, +1 to +65, +1 to +66, +1 to +67, +1 to +68, +1 to +69, +1 to +70, +1 to +71, +1 to +72, +1 to +73, +1 to +74, +1 to +75, +1 to +76, +1 to +77, +1 to +78, +1 to +79, +1 to +80, +1 to +81, +1 to +82, +1 to +83, +1 to +84, +1 to +85, +1 to +86, +1 to +87, +1 to +88, +1 to +89, +1 to +90, +1 to +90, +1 to +91, +1 to +92, +1 to +93, +1 to +94, +1 to +95, +1 to +96, +1 to +97, +1 to +98, +1 to +99, +1 to +100, +1 to +101, +1 to +102, +1 to +103, +1 to +104, +1 to +105, +1 to +106, +1 to +107, +1 to +108, +1 to +109, +1 to +110, +1 to +111, +1 to +112, +1 to +113, +1 to +114, +1 to +115, +1 to +116, +1 to +117, +1 to +118, +1 to +119, +1 to +120, +1 to +121, +1 to +122, +1 to +123, +1 to +124, or +1 to +125 from the nick site.

In still other embodiments, the desired nucleotide change is installed in an editing window that is between about +1 to +2 from the nick site, or about +1 to +5, +1 to +10, +1 to +15, +1 to +20, +1 to +25, +1 to +30, +1 to +35, +1 to +40, +1 to +45, +1 to +50, +1 to +55, +1 to +100, +1 to +105, +1 to +110, +1 to +115, +1 to +120, +1 to +125, +1 to +130, +1 to +135, +1 to +140, +1 to +145, +1 to +150, +1 to +155, +1 to +160, +1 to +165, +1 to +170, +1 to +175, +1 to +180, +1 to +185, +1 to +190, +1 to +195, or +1 to +200, from the nick site.

In various aspects, the extended guide RNAs are modified versions of an extended guide RNA. pegRNAs (i.e., extended guide RNAs) and ngRNAs may be expressed from an encoding nucleic acid, or synthesized chemically. Methods are well known in the art for obtaining or otherwise synthesizing guide RNAs, and for determining the appropriate sequence of the pegRNA, including the protospacer sequence, which interacts and hybridizes with the target strand of a genomic target site of interest.

In various embodiments, the particular design aspects of a pegRNA sequence and ngRNA sequence will depend upon the nucleotide sequence of a genomic target site of interest (i.e., the desired site to be edited) and the type of napDNAbp (e.g., Cas9 protein) present in the prime editing systems utilized in the methods and compositions described herein, among other factors, such as PAM sequence locations, percent G/C content in the target sequence, the degree of microhomology regions, secondary structures, etc.

In general, a spacer sequence (i.e., a guide sequence) of a pegRNA or ngRNA can be any polynucleotide sequence having sufficient complementarity with a target polynucleotide sequence to hybridize with the target sequence and direct sequence-specific binding of a napDNAbp (e.g., a Cas9, Cas9 homolog, or Cas9 variant) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

In some embodiments, a guide sequence is less than about 75, 50, 45, 40, 35, 30, 25, 20, 15, 12, or fewer nucleotides in length. The ability of a guide sequence to direct sequence-specific binding of a prime editor to a target sequence may be assessed by any suitable assay. For example, the components of a prime editor, including the guide sequence to be tested, may be provided to a host cell having the corresponding target sequence, such as by transfection with vectors encoding the components of a prime editor disclosed herein, followed by an assessment of preferential cleavage within the target sequence, such as by Surveyor assay as described herein. Similarly, cleavage of a target polynucleotide sequence may be evaluated in a test tube by providing the target sequence, components of a prime editor, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG where NNNNNNNNNNNNXGG (N is A, G, T, or C; and X can be anything). A unique target sequence in a genome may include an S. pyogenes Cas9 target site of the form MMMMMMMMMNNNNNNNNNNNXGG where NNNNNNNNNNNXGG (N is A, G, T, or C; and X can be anything). For the S. thermophilus CRISPR1Cas9, a unique target sequence in a genome may include a Cas9 target site of the form MMMMMMMMNNNNNNNNNNNNXXAGAAW where NNNNNNNNNNNNXXAGAAW (N is A, G, T, or C; X can be anything; and W is A or T). A unique target sequence in a genome may include an S. thermophilus CRISPR 1 Cas9 target site of the form MMMMMMMMMNNNNNNNNNNNXXAGAAW where NNNNNNNNNNNXXAGAAW (N is A, G, T, or C; X can be anything; and W is A or T). For the S. pyogenes Cas9, a unique target sequence in a genome may include a Cas9 target site of the form MMMMMMMMNNNNNNNNNNNNXGGXG where NNNNNNNNNNNNXGGXG (N is A, G, T, or C; and X can be anything). A unique target sequence in a genome may include an S. pyogenes Cas9 target site of the form MMMMMMMMMNNNNNNNNNNNXGGXG where NNNNNNNNNNNXGGXG (N is A, G, T, or C; and X can be anything). In each of these sequences “M” may be A, G, T, or C, and need not be considered in identifying a sequence as unique.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see, e.g., A. R. Gruber et al., 2008, Cell 106(1): 23-24; and P A Carr and G M Church, 2009, Nature Biotechnology 27(12): 1151-62). Further algorithms may be found in U.S. Application Ser. No. 61/836,080, incorporated herein by reference. In some embodiments, silent mutations are introduced in a guide sequence in order to alter its secondary structure and increase the efficiency of prime editing.

In some embodiments, the scaffold or gRNA core portion of a pegRNA comprises sequences corresponding to the tracr sequence and tracr mate sequence of a traditional guide RNA. In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides. Further non-limiting examples of single polynucleotides comprising a guide sequence, a tracr mate sequence, and a tracr sequence are as follows (listed 5′ to 3′), where “N” represents a base of a guide sequence, and the final poly-T sequence represents the transcription terminator:

(1)
(SEQ ID NO: 113)
NNNNNNNNGTTTTTGTACTCTCAAGATTTAGAAATAAATCTTGCAGAAG
CTACAAAGATAAGGCTTCATGCCGAAATCAACACCCTGTCATTTTATGG
CAGGGTGTTTTCGTTATTTAATTTTTT;
(2)
(SEQ ID NO: 114)
NNNNNNNNNNNNNNNNNNGTTTTTGTACTCTCAGAAATGCAGAAGCTACA
AAGATAAGGCTTCATGCCGAAATCAACACCCTGTCATTTTATGGCAGGGT
GTTTTCGTTATTTAATTTTTT;
(3)
(SEQ ID NO: 115)
NNNNNNNNNNNNNNNNNNNNGTTTTTGTACTCTCAGAAATGCAGAAGCTA
CAAAGATAAGGCTTCATGCCGAAATCAACACCCTGTCATTTTATGGCAGG
GTGTTTTTT;
(4)
(SEQ ID NO: 116)
NNNNNNNNNNNNNNNNNNNNGTTTTAGAGCTAGAAATAGCAAGTTAAAAT
AAGGCTAGTCCGTTATCAACTTGAAAAAGTGGCACCGAGTCGGTGCTTTT
TT;
(5)
(SEQ ID NO: 117)
NNNNNNNNNNNNNNNNNNNNGTTTTAGAGCTAGAAATAGCAAGTTAAAAT
AAGGCTAGTCCGTTATCAACTTGAAAAAGTGTTTTTTT;
and
(6)
(SEQ ID NO: 118)
NNNNNNNNNNNNNNNNNNNNGTTTTAGAGCTAGAAATAGCAAGTTAAAAT
AAGGCTAGTCCGTTATCATTTTTTTT.

In some embodiments, sequences (1) to (3) are used in combination with Cas9 from S. thermophilus CRISPR1. In some embodiments, sequences (4) to (6) are used in combination with Cas9 from S. pyogenes. In some embodiments, the tracr sequence is a separate transcript from a transcript comprising the tracr mate sequence.

It will be apparent to those of skill in the art that in order to target any of the fusion proteins comprising a Cas9 domain and a single-stranded DNA binding protein, as disclosed herein, to a target site, e.g., a site at which a recombinase recognition sequence is to be introduced, it is typically necessary to co-express the fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein.

In some embodiments, a pegRNA comprises a structure 5′-[guide sequence]-GUUUUAGAGCUAGAAAUAGCAAGUUAAAAUAAAGGCUAGUCCGUUAUCAACU UGAAAAAGUGGCACCGAGUCGGUGCUUUUU (SEQ ID NO: 119)-extension arm-3′, wherein the guide sequence comprises a sequence that is complementary to the target sequence. The guide sequence, also referred to herein as the spacer sequence, is typically 20 nucleotides long. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic acid sequence within 50 nucleotides upstream or downstream of the target nucleotide to be edited. Some exemplary guide RNA sequences suitable for targeting any of the provided fusion proteins to specific target sequences are provided herein. Additional guide sequences are well known in the art and can be used with the prime editors utilized in the methods and compositions described herein.

In some embodiments, a PEgRNA comprises three main component elements ordered in the 5′ to 3′ direction, namely: a spacer, a gRNA core, and an extension arm at the 3′ end. In some embodiments, the extension arm may further be divided into the following structural elements in the 5′ to 3′ direction, namely: an edit template, a homology arm, and a primer binding site. In some embodiments, the extension arm may further be divided into the following structural elements in the 5′ to 3′ direction, namely: a homology arm, an edit template, and a primer binding site. In some embodiments, the extension arm may further be divided into the following structural elements in the 5′ to 3′ direction, namely: a DNA synthesis template (e.g., a RT template), and a primer binding site. In addition, the PEgRNA may comprise an optional 3′ end modifier region and an optional 5′ end modifier region. Still further, the PEgRNA may comprise a transcriptional termination signal at the 3′ end of the PEgRNA. These structural elements are further defined herein. The depiction of the structure of the PEgRNA is not meant to be limiting and embraces variations in the arrangement of the elements. For example, the optional sequence modifiers and could be positioned within or between any of the other regions shown, and not limited to being located at the 3′ and 5′ ends.

### PEgRNA Modifications

The PEgRNAs may also include additional design modifications that may alter the properties and/or characteristics of PEgRNAs, thereby improving the efficacy of prime editing. In various embodiments, these modifications may belong to one or more of a number of different categories, including but not limited to: (1) designs to enable efficient expression of functional PEgRNAs from non-polymerase III (pol III) promoters, which would enable the expression of longer PEgRNAs without burdensome sequence requirements; (2) modifications to the core, Cas9-binding PEgRNA scaffold, which could improve efficacy; (3) modifications to the PEgRNA to improve RT processivity, allowing the insertion of longer sequences at targeted genomic loci; and (4) addition of RNA motifs to the 5′ or 3′ termini of the PEgRNA that improve PEgRNA stability, enhance RT processivity, prevent misfolding of the PEgRNA, or recruit additional factors important for genome editing. Such modifications are described further, for example, in PCT publication WO 2022/067130, which is incorporated herein by reference.

### Pharmaceutical Compositions

Other aspects of the present disclosure relate to pharmaceutical compositions comprising any of the reverse transcriptase variants, Cas9 variants, prime editors, fusion proteins, or complexes provided herein, or any of the polynucleotides or vectors encoding such reverse transcriptase variants, Cas9 variants, prime editors, fusion proteins, or complexes provided herein. The term “pharmaceutical composition,” as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds). In some embodiments, the pharmaceutical composition further comprises a pegRNA, or a polynucleotide encoding a pegRNA.

As used herein, the term “pharmaceutically-acceptable carrier” means a pharmaceutically acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the protein, fusion protein, polynucleotide, or vector from one site (e.g., the delivery site) of the body, to another site (e.g., an organ, tissue, or other part of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials that can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL, and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservatives, and antioxidants can also be present in the formulation. The terms such as “excipient,” “carrier,” “pharmaceutically acceptable carrier,” or the like are used interchangeably herein.

In some embodiments, the pharmaceutical composition is formulated for delivery to a subject, e.g., for gene editing. Suitable routes of administering the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, the pharmaceutical composition is formulated in accordance with routine procedures as a composition adapted for intravenous or subcutaneous administration to a subject, e.g., a human. In some embodiments, pharmaceutical compositions for administration by injection are solutions in sterile isotonic aqueous buffer. Where necessary, the pharmaceutical composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical composition is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Proteins, fusion proteins, polynucleotides, or vectors can be entrapped in “stabilized plasmid-lipid particles” (SPLP) containing the fusogenic lipid dioleoylphosphatidylethanolamine (DOPE), low levels (5-10 mol %) of cationic lipid and stabilized by a polyethyleneglycol (PEG) coating (Zhang Y. P. et al., Gene Ther. 1999, 6:1438-47). Positively charged lipids, such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical compositions described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent, i.e., carrier or vehicle.

Further, the pharmaceutical composition can be provided as a pharmaceutical kit comprising (a) a container containing a protein, fusion protein, complex (e.g., ribonucleoprotein complex), polynucleotide, or vector of the invention in lyophilized form and (b) a second container containing a pharmaceutically acceptable diluent (e.g., sterile water) for injection. The pharmaceutically acceptable diluent can be used for reconstitution or dilution of the lyophilized protein, fusion protein, complex (e.g., ribonucleoprotein complex), polynucleotide, or vector of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials, such as glass or plastic. In some embodiments, the container holds a composition that is effective for treating a disease and may have a sterile access port. For example, the container may be an intravenous solution bag or a vial having a stopper pierce-able by a hypodermic injection needle. The active agent in the composition is a protein, fusion protein, polynucleotide, or vector of the invention. In some embodiments, the label on or associated with the container indicates that the composition is used for treating the disease of choice. The article of manufacture may further comprise a second container comprising a pharmaceutically acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Polynucleotides, Vectors, AAVs, Kits, and Cells

In some aspects, the present disclosure provides polynucleotides and vectors encoding any of the reverse transcriptase variants, Cas9 variants, fusion proteins, or prime editors provided herein. In some aspects, the present disclosure provides one or more polynucleotides and vectors encoding any of the complexes provided herein. In some embodiments, the polynucleotides and vectors provided herein comprise DNA. In some embodiments, the polynucleotides and vectors provided herein comprise RNA. In some aspects, any of the polynucleotides described herein may be provided in a vector.

In some embodiments, one or more polynucleotides encoding any of the complexes provided herein are delivered to a cell, e.g., using an AAV. In certain embodiments, two polynucleotides encoding any of the complexes provided herein are delivered to a cell, e.g., using an AAV. In certain embodiments, the two polynucleotides comprise two halves of a prime editor described herein and comprising a split intein capable of reassembling into a prime editor molecule. In some embodiments, the one or more polynucleotides encoding a complex provided herein are delivered to a cell in one or more adeno-associated virus (AAV) particles. Delivery of prime editor complexes has been described, for example, in U.S. Provisional application, U.S. Ser. No., 63/426,336, filed Nov. 17, 2022, U.S. Provisional application, U.S. Ser. No., 63/491,013, filed Mar. 17, 2023, and Davis, J. R., et al. Nat. Biotechnol. 2023, each of which is incorporated herein by reference. In certain embodiments, the one or more polynucleotides encoding the complex are delivered to the cell in two AAV particles. In some embodiments, one or both of the AAV particles comprise AAV1, AAV2, AAV3, AAV4, AAV5, AAV6, AAV7, AAV8, or AAV9. In certain embodiments, one or both of the AAV particles comprise AAV9.

In some embodiments, a first and second AAV particle are delivered to a cell. In certain embodiments, the first AAV particle comprises a polynucleotide comprising the structure 5′-[inverted terminal repeat (ITR) sequence]-[promoter]-[napDNAbp N-terminal fragment]-[N-intein]-[terminator sequence]-[ITR sequence]-3′. In certain embodiments, the second AAV particle comprises a polynucleotide comprising the structure 5′-[ITR sequence]-[promoter]-[C-intein]-[napDNAbp C-terminal fragment]-[reverse transcriptase]-[terminator sequence]-[optional nicking gRNA]-[pegRNA]-[ITR]-3′.

The reverse transcriptase variants, Cas9 variants, prime editors, fusion proteins, or complexes provided herein may also be assembled into kits. In some embodiments, the kit comprises polynucleotides for expression of any of the reverse transcriptase variants, Cas9 variants, prime editors, or complexes provided herein. In other embodiments, the kit further comprises appropriate pegRNAs or nucleic acid vectors for the expression of such pegRNAs.

The kits described herein may include one or more containers housing components for performing the methods described herein, and optionally instructions for use. In some embodiments, the kits include instructions for editing a particular disease or treating a particular disease by prime editing. In some embodiments, the kits include instructions for editing a particular gene in a cell. Any of the kits described herein may further comprise components needed for performing any of the methods described herein. Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), internet, and/or web-based communications, etc. The written instructions may be in a form prescribed by a governmental agency regulating the manufacture, use, or sale of pharmaceuticals or biological products, which can also reflect approval by the agency of manufacture, use, or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral, and electronic communication of any form, associated with the disclosure. Additionally, the kits may include other components depending on the specific application, as described herein.

The kits may contain any one or more of the components described herein in one or more containers. The components may be prepared sterilely, packaged in a syringe, and shipped refrigerated. Alternatively, they may be housed in a vial or other container for storage. A second container may have other components prepared sterilely. Alternatively, the kits may include the active agents premixed and shipped in a vial, tube, or other container.

The kits may have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box, or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc. Some aspects of this disclosure provide kits comprising a nucleic acid construct comprising a nucleotide sequence encoding the prime editor systems described herein, or various components thereof (e.g., the reverse transcriptase variants, Cas9 variants, prime editors, complexes, polynucleotides, and/or vectors provided herein). In some embodiments, the nucleotide sequence(s) comprises a heterologous promoter (or more than a single promoter) that drives expression of one or more prime editor system components.

Cells that may contain any of the reverse transcriptase variants, Cas9 variants, prime editors, fusion proteins, complexes, polynucleotides, and/or vectors described herein include prokaryotic cells and eukaryotic cells. The methods described herein may be used to deliver a pegRNA and a prime editor into a eukaryotic cell (e.g., a mammalian cell, such as a human cell). In some embodiments, the cell is in vitro (e.g., a cultured cell). In some embodiments, the cell is in vivo (e.g., in a subject, such as a human subject). In some embodiments, the cell is ex vivo (e.g., isolated from a subject and may be administered back to the same or a different subject).

Some aspects of this disclosure provide cells comprising any of the vectors or other constructs disclosed herein. In some embodiments, a host cell is transiently or non-transiently transfected or electroporated with one or more vectors described herein. In some embodiments, a cell is transfected or electroporated as it naturally occurs in a subject. In some embodiments, a cell that is transfected or electroporated is taken from a subject. In some embodiments, the cell is derived from cells taken from a subject, such as a cell line.

### Methods of Use

In some aspect, the present disclosure provides methods for editing a nucleic acid molecule by prime editing comprising contacting a nucleic acid molecule with prime editors or complexes (or polynucleotides or vectors encoding the same), thereby installing one or more modifications to the nucleic acid molecule at a target site. Prime editing refers to an approach for gene editing using napDNAbps, a polymerase (e.g., a reverse transcriptase), and specialized guide RNAs that include a primer binding site and a DNA synthesis template for encoding desired new genetic information (or deleting genetic information) that is then incorporated into a target DNA sequence. For example, prime editing may be used to incorporate one or more recombinase recognition sequences into target DNA sequence such as a genome, as described herein. Prime editing is described in Anzalone, A. V. et al., Search-and-replace genome editing without double-strand breaks or donor DNA. Nature 576, 149-157 (2019), which is incorporated herein by reference. See also International PCT application, PCT/US2020/023721, filed Mar. 19, 2020, and published as WO 2020/191239, which is incorporated herein by reference.

Prime editing represents a platform for genome editing that is a versatile and precise method to directly write new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit (e.g., a recombinase recognition sequence to be inserted into a target DNA) in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand (or is homologous to it) immediately downstream of the nick site of the target site to be edited (with the exception that it includes the desired edit). Through DNA repair and/or replication machinery, the endogenous strand downstream of the nick site is replaced by the newly synthesized replacement strand containing the desired edit. In some cases, prime editing may be thought of as a “search-and-replace” genome editing technology since the prime editors, as described herein, not only search and locate the desired target site to be edited, but at the same time, encode a replacement strand containing a desired edit that is installed in place of the corresponding target site endogenous DNA strand. The prime editors of the present disclosure relate, in part, to the discovery that the mechanism of target-primed reverse transcription (TPRT) or “prime editing” can be leveraged or adapted for conducting precision CRISPR/Cas-based genome editing with high efficiency and genetic flexibility. TPRT is naturally used by mobile DNA elements, such as mammalian non-LTR retrotransposons and bacterial Group II introns. Cas protein-reverse transcriptase fusions or related systems are used to target a specific DNA sequence with a guide RNA, generate a single strand nick at the target site, and use the nicked DNA as a primer for reverse transcription of an engineered DNA synthesis template that is integrated with the guide RNA. However, while the concept begins with prime editors that use reverse transcriptase as the DNA polymerase component, the prime editors described herein are not limited to reverse transcriptases but may include the use of virtually any DNA polymerase. Indeed, while the application throughout may refer to prime editors with “reverse transcriptases,” it is set forth here that reverse transcriptases are only one type of DNA polymerase that may work with prime editing. Thus, wherever the specification mentions a “reverse transcriptase,” the person having ordinary skill in the art should appreciate that any suitable DNA polymerase may be used in place of the reverse transcriptase. Thus, in one aspect, the prime editors may comprise Cas9 (or an equivalent napDNAbp), which is programmed to target a DNA sequence by associating it with a specialized guide RNA (i.e., PEgRNA) containing a spacer sequence that anneals to a complementary sequence (the complementary sequence to an endogenous protospacer sequence) in the target DNA. The PEgRNA also contains new genetic information in the form of an extension that encodes a replacement strand of DNA containing a desired nucleotide change which is used to replace a corresponding endogenous DNA strand at the target site. To transfer information from the PEgRNA to the target DNA, the mechanism of prime editing involves nicking the target site in one strand of the DNA to expose a 3′-hydroxyl group. The exposed 3′-hydroxyl group can then be used to prime the DNA polymerization of the edit-encoding extension on PEgRNA directly into the target site. In various embodiments, the extension—which provides the template for polymerization of the replacement strand containing the edit—can be formed from RNA or DNA. In the case of an RNA extension, the polymerase of the prime editor can be an RNA-dependent DNA polymerase (such as a reverse transcriptase). In the case of a DNA extension, the polymerase of the prime editor may be a DNA-dependent DNA polymerase. The newly synthesized strand (i.e., the replacement DNA strand containing the desired nucleotide edit) that is formed by the prime editor would be homologous to the genomic target sequence (i.e., have the same sequence as), except for the inclusion of one or more desired nucleotide changes (e.g., a single nucleotide substitution, a deletion, or an insertion, or a combination thereof). The newly synthesized (or replacement) strand of DNA may also be referred to as a single strand DNA flap, which would compete for hybridization with the complementary homologous endogenous DNA strand, thereby displacing the corresponding endogenous strand. Resolution of the hybridized intermediate (also referred to as a heteroduplex, comprising the single strand DNA flap synthesized by the reverse transcriptase hybridized to the endogenous DNA strand with the exception of mismatches at positions where desired nucleotide edits are installed in the edit strand) can include removal of the resulting displaced flap of endogenous DNA (e.g., with a 5′ end DNA flap endonuclease, FEN1), ligation of the synthesized single strand DNA flap to the target DNA, and assimilation of the desired nucleotide changes as a result of cellular DNA repair and/or replication processes. Because templated DNA synthesis offers single nucleotide precision for the modification of any nucleotide, including insertions and deletions, the scope of this approach is very broad and could foreseeably be used for myriad applications in basic science and therapeutics. In certain embodiments, the system can be combined with the use of an error-prone reverse transcriptase enzyme (e.g., provided as a fusion protein with the Cas9 domain, or provided in trans to the Cas9 domain). The error-prone reverse transcriptase enzyme can introduce alterations during synthesis of the single strand DNA flap. Thus, in certain embodiments, error-prone reverse transcriptase can be utilized to introduce nucleotide changes to the target DNA. Depending on the error-prone reverse transcriptase that is used with the system, the changes can be random or non-random.

In various embodiments, prime editing operates by contacting a target DNA molecule (for which a change in the nucleotide sequence is desired to be introduced) with a nucleic acid programmable DNA binding protein (napDNAbp) complexed with a prime editing guide RNA (PEgRNA). In various embodiments, the prime editing guide RNA (PEgRNA) comprises an extension at the 3′ or 5′ end of the guide RNA, or at an intramolecular location in the guide RNA, and encodes the desired nucleotide change (e.g., single nucleotide substitution, insertion, or deletion). First, the napDNAbp/extended gRNA complex contacts the DNA molecule, and the extended gRNA guides the napDNAbp to bind to a target locus. Next, a nick in one of the strands of DNA of the target locus is introduced (e.g., by a nuclease or chemical agent), thereby creating an available 3′ end in one of the strands of the target locus. In certain embodiments, the nick is created in the strand of DNA that corresponds to the R-loop strand, i.e., the strand that is not hybridized to the guide RNA sequence, i.e., the “non-target strand.” The nick, however, could be introduced in either of the strands. That is, the nick could be introduced into the R-loop “target strand” (i.e., the strand hybridized to the protospacer of the extended gRNA) or the “non-target strand” (i.e., the strand forming the single-stranded portion of the R-loop and which is complementary to the target strand). In the next step, the 3′ end of the DNA strand (formed by the nick) interacts with the extended portion of the guide RNA in order to prime reverse transcription (i.e., “target-primed RT”). In certain embodiments, the 3′ end DNA strand hybridizes to a specific RT priming sequence on the extended portion of the guide RNA, i.e., the “reverse transcriptase priming sequence” or “primer binding site” on the PEgRNA. In the next step, a reverse transcriptase (or other suitable DNA polymerase) is introduced that synthesizes a single strand of DNA from the 3′ end of the primed site towards the 5′ end of the prime editing guide RNA. The DNA polymerase (e.g., reverse transcriptase) can be fused to the napDNAbp or alternatively can be provided in trans to the napDNAbp. This forms a single-strand DNA flap comprising the desired nucleotide change (e.g., the single base change, insertion, or deletion, or a combination thereof, for example, a recombinase recognition sequence to be inserted into a target DNA sequence such as a genome) and that is otherwise homologous to the endogenous DNA at or adjacent to the nick site. In the next step, the napDNAbp and guide RNA are released. The final two steps relate to the resolution of the single strand DNA flap such that the desired nucleotide change becomes incorporated into the target locus. This process can be driven towards the desired product formation by removing the corresponding 5′ endogenous DNA flap that forms once the 3′ single strand DNA flap invades and hybridizes to the endogenous DNA sequence. Without being bound by theory, the cell's endogenous DNA repair and replication processes resolve the mismatched DNA to incorporate the nucleotide change(s) to form the desired altered product. The process can also be driven towards product formation with “second strand nicking.” This process may introduce at least one or more of the following genetic changes: transversions, transitions, deletions, and insertions (e.g., insertion of a recombinase recognition sequence). In some embodiments, one or more recombinase recognition sequences are inserted into a target DNA sequence using prime editing, and then these recombinase recognition sequences are contacted with a recombinase (e.g., any of the evolved recombinases provided herein) and, optionally, a donor DNA sequence to be inserted into the target DNA sequence.

The term “prime editor (PE) system” or “prime editor (PE)” or “PE system” or “PE editing system” refers the compositions involved in the method of genome editing using target-primed reverse transcription (TPRT) described herein, including, but not limited to, the napDNAbps, reverse transcriptases, fusion proteins (e.g., comprising napDNAbps and reverse transcriptases), prime editing guide RNAs, and complexes comprising fusion proteins and prime editing guide RNAs, as well as accessory elements, such as second strand nicking components (e.g., second strand nicking sgRNAs) and 5′ endogenous DNA flap removal endonucleases (e.g., FEN1) for helping to drive the prime editing process towards the edited product formation.

Although in the embodiments described thus far the PEgRNA constitutes a single molecule comprising a guide RNA (which itself comprises a spacer sequence and a gRNA core or scaffold) and a 5′ or 3′ extension arm comprising the primer binding site and a DNA synthesis template, the PEgRNA may also take the form of two individual molecules. For example, in some embodiments, a PEgRNA may comprise a guide RNA and a trans prime editor RNA template (tPERT), which essentially houses the extension arm (including, in particular, the primer binding site and the DNA synthesis domain) and an RNA-protein recruitment domain (e.g., MS2 aptamer or hairpin) in the same molecule which becomes co-localized or recruited to a modified prime editor complex that comprises a tPERT recruiting protein (e.g., MS2cp protein, which binds to the MS2 aptamer).

A prime editor system can comprise one or more prime editing guide RNAs (PEgRNAs). In some embodiments, a prime editor system has one PEgRNA (the “single flap prime editing system”) that targets one strand of a double stranded DNA, e.g., a target genomic site. For example, a single flap prime editing system may comprise a spacer sequence that comprises complementarity to a target strand of a double stranded target DNA, a primer binding site that comprises complementarity to a non-target strand of the double stranded target DNA, and a DNA synthesis template that comprises (and encodes) a nucleotide edit compared to the double stranded target DNA sequence, e.g., a recombinase recognition site. In some embodiments, a prime editor system (the “dual-flap prime editing system” or “twin prime editing” or “twinPE”) comprises at least two different PEgRNAs that can target opposite strands of a double stranded target DNA, e.g., a target genomic site. For example, a twin prime editing system may comprise two PEgRNAs, wherein each of the two PEgRNAs comprises a DNA synthesis template having a region of complementarity to each other, and direct the synthesis of two 3′ flaps having a region of complementarity to each other and contains a nucleotide edit compared to the double stranded target DNA sequence, (e.g., a recombinase recognition sequence). Unlike single flap prime editing, there is no requirement for the pair of edited DNA strands (3′ flaps) to directly compete with 5′ flaps in endogenous genomic DNA (i.e., no requirement for a homology arm in the extension arm which would generate a region having complementarity to the endogenous DNA), as the complementary edited strand is available for hybridization instead. Since both strands of the duplex are synthesized as edited DNA, the dual-flap prime editing system obviates the need for the replacement of the non-edited complementary DNA strand required by classical prime editing. Instead, cellular DNA repair machinery need only excise the paired 5′ flaps (original genomic DNA) and ligate the paired 3′ flaps (edited DNA) into the locus. Therefore, there is also no need to include sequences homologous to genomic DNA in the newly synthesized DNA strands, allowing selective hybridization of the new strands and facilitating edits that contain minimal genomic homology. Nuclease-active versions of prime editors that cut both strands of DNA could also be used to accelerate the removal of the original DNA sequence.

Thus, in some aspects, the present disclosure provides methods for simultaneously editing both strands of a double-stranded nucleic acid molecule by twin prime editing at a target site to be edited comprising contacting the double-stranded nucleic acid molecule with: (a) any of the prime editors disclosed herein (or one or more polynucleotides encoding the same); (b) a first prime editing guide RNA (first pegRNA), or a polynucleotide encoding the first pegRNA that comprises (i) a first spacer sequence that binds to a first binding site on a first strand of the double-stranded DNA sequence upstream of the target site relative to the second strand, (ii) a first gRNA core that is capable of complexing with the prime editor, and (iii) a first DNA synthesis template that encodes a first single-stranded DNA sequence, and (c) a second prime editing guide RNA (second pegRNA), or a polynucleotide encoding the second pegRNA, that comprises (i) a second spacer sequence that binds to a second binding site on a second strand of the double-stranded DNA sequence downstream of the target site relative to the second strand; (ii) a second gRNA core that is capable of complexing with the prime editor, and (iii) a second DNA synthesis template that encodes a second single-stranded DNA sequence.

Variants of twin prime editing include quadruple-flap prime editing whereby the two sets of twin prime editors are used to introduce a genetic change at two different genetic loci, e.g., two different recombinase recognition sequences located at the 5′ end and 3′ end of a gene.

Like classical prime editing, twin prime editing (including dual-flap and quadruple-flap prime editing) is a versatile and precise genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a recombinase recognition sequence for insertion into a target DNA sequence) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit.

In some embodiments, the methods provided herein comprise installing a recombinase recognition site in a target nucleic acid molecule. In some embodiments, the methods provided herein combine the use of prime editing (e.g., using any of the prime editors described herein) with site-specific recombination. In some embodiments, such methods facilitate insertion of large DNA (e.g., whole genes), into the genome of an organism (for example, in the CNS).

The term “site-specific recombination” refers to a type of genetic recombination also known as “conservative site-specific recombination.” Site-specific recombination is a type of genetic recombination in which DNA strand exchange takes place between segments possessing at least a certain degree of sequence homology. Enzymes known as site-specific recombinases (“SSRs”), such as Bxb1, perform rearrangements of DNA segments by recognizing and binding to short, specific DNA sequence (“recombinase recognition sites”), at which they cleave the DNA backbone, exchange the two DNA helices involved, and rejoin the DNA strands. In some cases, the presence of a recombinase enzyme and the recombination sites is sufficient for the reaction to proceed; in other systems a number of accessory proteins and/or accessory sites are required. Many different genome modification strategies, among these recombinase-mediated cassette exchange (RMCE), an advanced approach for the targeted introduction of transcription units into predetermined genomic loci, rely on SSRs. Site-specific recombination systems are highly specific, fast, and efficient, even when faced with complex eukaryotic genomes. They are employed naturally in a variety of cellular processes, including bacterial genome replication, differentiation and pathogenesis, and movement of mobile genetic elements. Recombination sites are typically between about 30 and 200 nucleotides in length and generally consist of two motifs with a partial inverted-repeat symmetry, to which the recombinase binds, and which flank a central crossover sequence at which the recombination takes place. The pairs of sites between which the recombination occurs are usually identical, but there are exceptions (e.g., attP and attB).

Once a recombinase recognition site is installed in the genome, a cognate recombinase that recognizes the installed recombinase recognition site may be used to catalyze the precise cleavage, strand exchange, and rejoining of DNA fragments at the defined recombinase recognition sites. This is accomplished without relying on endogenous repair mechanisms in a cell for repairing double-strand breaks that otherwise can induce indels and other undesirable DNA rearrangements. The reactions catalyzed by recombinases and recombinase recognition sites result in large-scale genomic changes, such as, insertions, deletions, inversions, replacements, and chromosomal translocations of one or more chromosomal regions, including one or more loci, one or more genes, or one or more portions of genes (e.g., gene exons, introns, and gene regulatory regions).

In certain embodiments, the one or more recombinase recognition sites can be inserted or introduced anywhere within a genome. In some organisms, a genome is organized as a single chromosome (e.g., bacteria) and the recombinase recognition site may be inserted at any locus within the chromosome. The insertion site may be within a gene or within an intergenic region of a chromosome. The insertion may be within an exon, intron, or therebetween, or within a regulatory sequence, such as a promoter, enhancer, or transcription binding sequence. In other organisms, e.g., humans, the genome is organized into more than one chromosome, and the recombinase recognition site may be inserted at any locus within the chromosome. For instance, in humans, the genome comprises 23 pairs of chromosomes. In addition, the genome also may be mitochondrial DNA. The insertion site may be within a gene or within an intergenic region of a chromosome. The insertion may be within an exon, intron, or therebetween, or within a regulatory sequence, such as a promoter, enhancer, or transcription binding sequence.

As used herein “inserting in a genome” in any organism can include inserting one or more SSR recognition sites in any one or more chromosomes of a given genome (depending upon the number of chromosomes making up the genome) and at any chromosomal locus or loci. Where a genome comprises more than one chromosome, reference to “inserting in a genome” may include inserting the one or more SSRs into the one or more chromosomes of the genome. For example, in humans—which have 23 pairs of chromosomes—reference to “inserting in a genome” refers to inserting one or more SSR recognition sites in any one of chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21, chromosome 22, or chromosome 23 (aka, XX chromosome or XY chromosome), or insertion into any combination of said chromosomes, or in a mitochondrial genome.

In various embodiments, the disclosure provides compositions and methods for installing one or more recombinase recognition sites using single flap prime editing (“classical PE”), twin prime editing (or twinPE), or multi-flap PE.

In some embodiments, classical PE may be used to insert one or more or two or more recombinase recognition sites into a desired genomic site.

In some embodiments, twinPE may be used to insert one or more or two or more recombinase recognition sites into a desired genomic site.

In some embodiments, multi-flap PE may be used to insert one or more or two or more recombinase recognition sites into one more desired genomic sites.

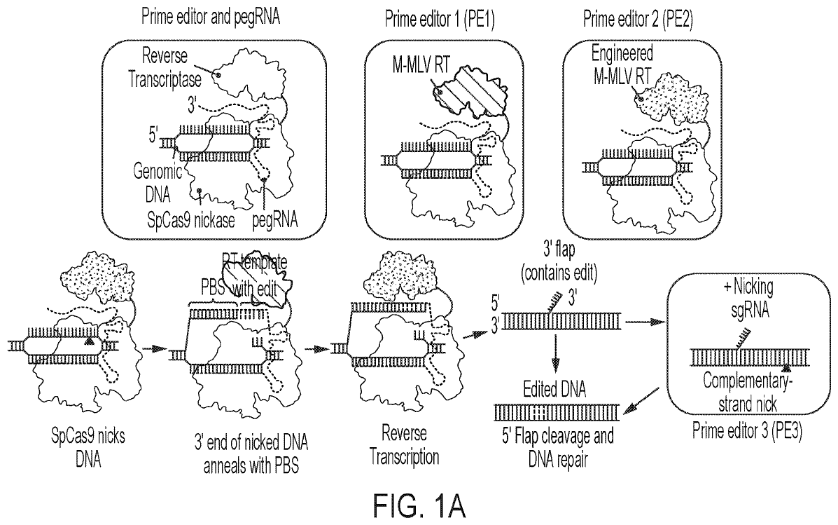

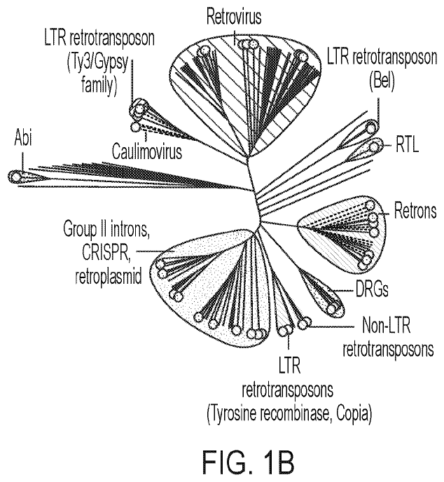

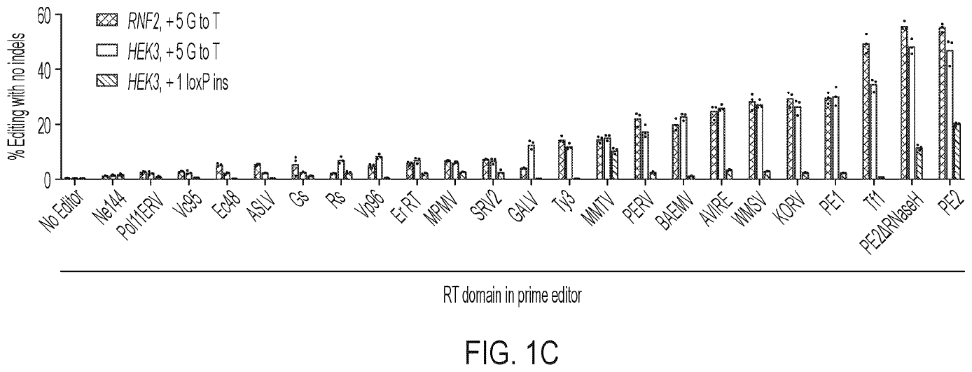

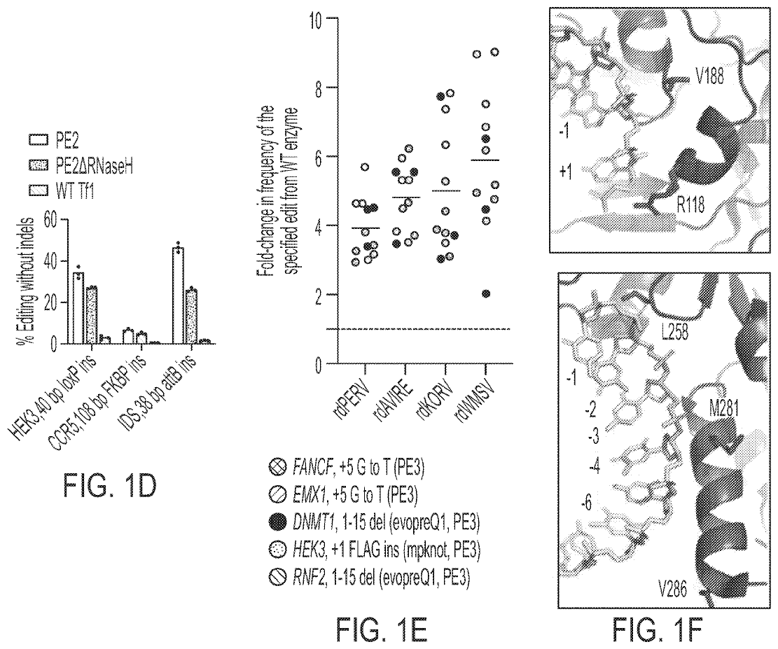

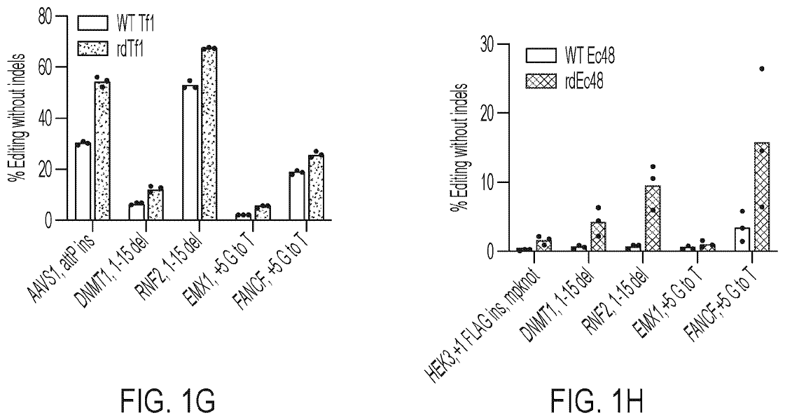

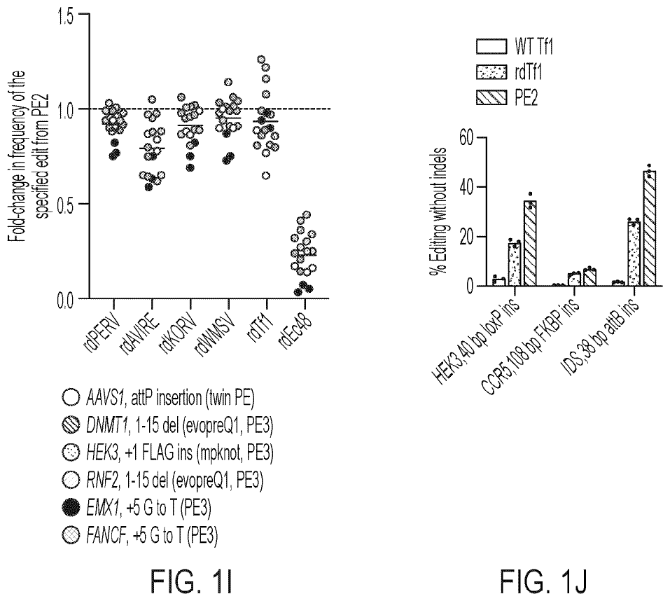

Insertion of recombinase recognition sites provides a programmed location for effecting one or more site-specific intended edits in a target DNA, e.g., genetic changes in a target gene or a genome. Non-limiting examples of intended edits via genetic recombination include insertion of an exogenous sequence into a target DNA, deletion (excision) of an endogenous sequence in a target DNA, inversion of an endogenous sequence in a target DNA, replacement of an endogenous sequence in a target DNA by an exogenous sequence, translocation of sequences between two target DNA sequences (e.g., between two different chromosomes), and any combination thereof. Accordingly, when the target DNA is a target gene or target genome, genetic changes via recombination can include, for example, genomic integration of an exogenous DNA sequence, e.g., sequence of a plasmid or a part thereof, genomic deletion or insertion, chromosomal translocations, and replacement of an endogenous genomic sequence in a target genome by an exogenous sequence (“cassette exchanges”), among other genetic changes. These exemplary types of genetic changes are illustrated in FIG. 1.

The mechanism of installing a recombinase recognition site into the genome is analogous to installing other sequences, such as peptide/protein and RNA tags, into the genome. Recombinase sites can be installed in a target DNA, e.g., a target genome, with single flap prime editing, twin prime editing, or multi-flap prime editing.

In some embodiments, any of the methods described herein are performed in vitro. In some embodiments, any of the methods described herein are performed ex vivo. In some embodiments, any of the methods described herein are performed in vivo. In some embodiments, any of the methods described herein are performed in a subject. In certain embodiments, the subject is a human. In some embodiments, editing a target nucleic acid using any of the methods described herein may be performed in order to treat a disease or disorder, for example, in a subject such as a human. In certain embodiments, the disease or disorder is Bloom syndrome, Crigler-Najjar disease, or Pomp disease. In some embodiments, the methods described herein may be used for systematic tagging of proteins (e.g., using PE6d).

The function and advantage of these and other embodiments of the present invention will be more fully understood from the Examples below. The following Examples are intended to illustrate the benefits of the present invention and to describe particular embodiments, but are not intended to exemplify the full scope of the invention. Accordingly, it will be understood that the Examples are not meant to limit the scope of the invention.

### EXAMPLES

### Example 1. Phage-Assisted Continuous Evolution and Protein Engineering Yield Compact, Efficient Prime Editors and Insights into Prime Editing

### Surveying Reverse Transcriptase Enzymes for Prime Editing

Although RTs are a diverse superfamily of enzymes, only a handful of enzymes beyond M-MLV RT variants have been used for prime editing, and these systems have been limited by low editing efficiencies^{4,17,23,24}. Thus, the development of improved RTs for PE began with a survey of RT enzymes from diverse phylogenetic origins. The activity of 59 novel RTs spanning 14 different classes (FIG. 1B) in the PE system at three different prime edits in HEK293T cells were tested and compared to PE1, PE2, and PE2ΔRNaseH (the RNaseH-truncated form of PE2 used for the dual-AAV delivery of prime editors). 20 novel, unique RTs with detectable prime editing activity were identified (FIG. 1C). Eleven of these enzymes are closely related to the M-MLV RT and are encoded by retroviruses, two are encoded by the Ty3/gypsy group of LTR retrotransposons, and seven are bacterial RTs encoded by group-II introns, retrons, or CRISPR-Cas associated systems. Nine of the 20 PE-compatible RTs are at least 500 bp smaller in gene size than M-MLV RT. All of these PE-compatible RTs, however, exhibited lower editing efficiencies than PE2, with the smaller RTs demonstrating especially poor activity (FIG. 1C and FIG. 81A). These results agree with recent reports^{4,17,23,24} and demonstrate that while diverse RT enzymes from nature can support detectable PE activity, in their wild-type forms they do not support robust mammalian cell prime editing.

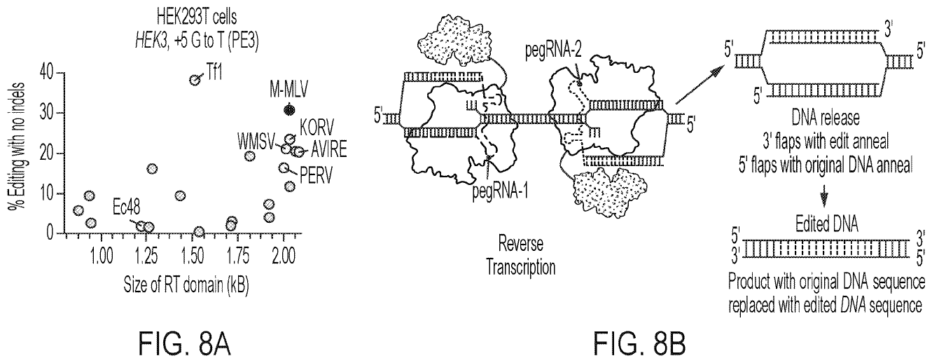

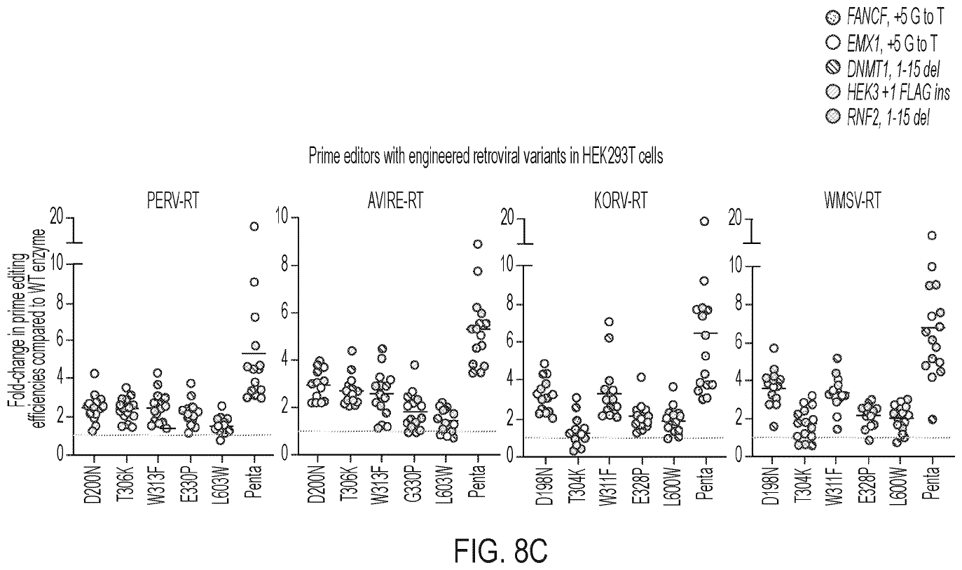

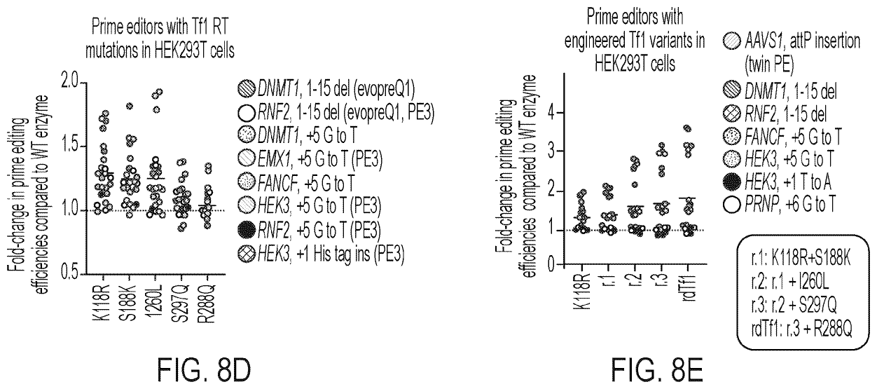

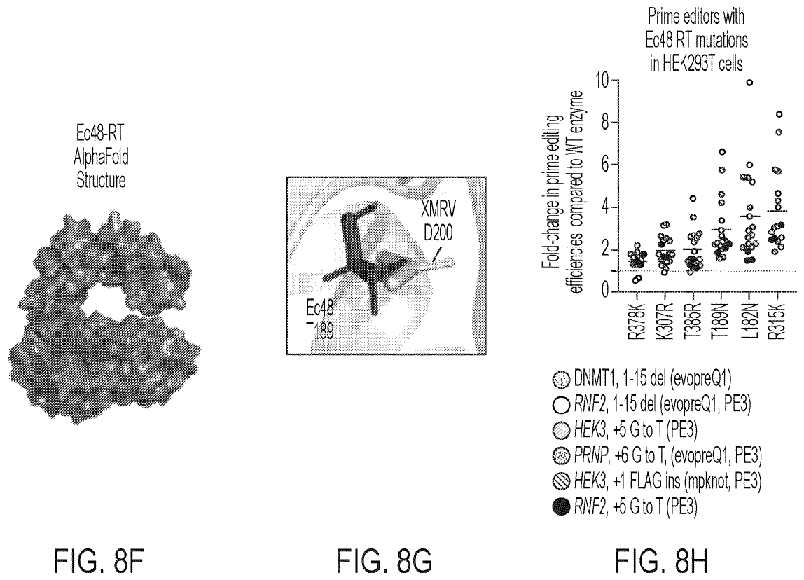

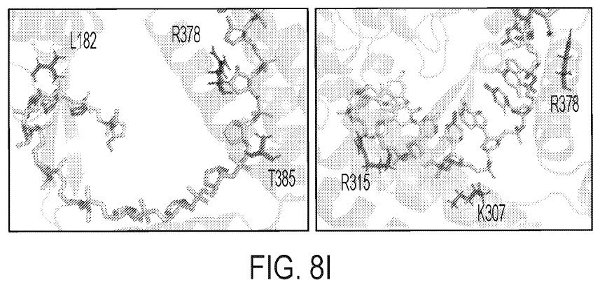

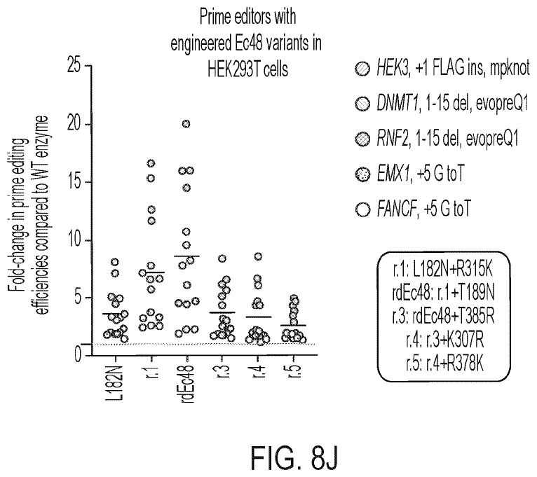

Of all the novel RTs tested, Tf1 RT exhibited the highest average prime editing efficiency across the three edits. Although wild-type Tf1 RT approached PE2 levels of editing efficiency at simple base substitution edits, it struggled to install a more challenging edit such as precise insertion of a 40-bp loxP sequence at the HEK3 locus. A similar trend was noted for the PE2ΔRNaseH construct, which uses the MMLV RT without an RNaseH domain for delivery purposes. Recent reports have shown that the RNaseH domain of the MMLV RT is dispensable for prime editing^{23,29,30}. However, data from FIG. 1C suggested that PE2ΔRNaseH might exhibit deficiencies at more challenging edits. To further test this hypothesis, PE2, Tf1, and PE2ΔRNaseH were compared at two additional complex edits that utilize twin prime editing (FIG. 8B). Both enzymes performed worse than PE2 for both additional edits. On average, at these three challenging edits PE2ΔRNaseH yielded 1.4-fold lower prime editing efficiency than PE2 and wild-type Tf1 performed 15-fold worse than PE2 (FIG. 1D).

These initial findings identified three challenges. The first was that the vast majority of non-M-MLV RTs, especially the most compact enzymes, were unable to support efficient mammalian prime editing, regardless of edit type. Second, even the most active dual AAV-compatible RTs (~1.5 kb in gene size) such as MMLVΔRNaseH showed deficiencies relative to full-length PEmax when installing long, complex edits. These smaller prime editors are important for many in vivo applications, as canonical prime editors such as PE3 or PEmax are too large to fit within dual-AAV delivery systems. Finally, none of the enzymes were able to surpass the editing efficiency of PEmax, and it was unclear what enzymatic preferences or bottlenecks in prime editing might limit the performance of PEmax.

### Rational Engineering of Reverse Transcriptase Enzymes

These challenges were first addressed using protein engineering. The PE2 protein contains five engineered mutations (D200N, T306K, W313F, T330P, and L603W) in M-MLV RT that enhance the enzyme's in vitro substrate binding, processivity, and thermostability^{1,18-21}. These mutations also substantially improve prime editing efficiencies in mammalian cells¹. The possibility that incorporating mutations homologous to those in PE2 into novel prime editors might improve their PE efficiencies was tested. Indeed, installing mutations corresponding to each of the five PE2 substitutions into RTs from porcine endogenous retrovirus (PERV), koala retrovirus (KoRV), avian reticuloendotheliosis virus (AVIRE), and woolly monkey sarcoma virus (WMSV) retroviruses resulted in increased prime editing efficiencies (FIG. 8C). Combining all five mutations further improved editing efficiencies by an average of 5.3-fold to 6.8-fold compared to the wild-type RTs for all enzymes across five different edits in HEK293T cells (FIG. 1E, FIG. 8C).

Simultaneously, it was sought to engineer the Tf1 RT due to its higher baseline performance compared to other WT enzymes and its small size. Given that increasing the affinity between the RT and its DNA⋅RNA substrate can improve PE efficiency¹, structure-guided engineering was first used to identify single-mutant variants that might improve RT-substrate binding. Using the structure of a Tf1 homolog, Ty3 RT, complexed with a DNA-RNA hybrid (Protein Data Bank (PDB): 40L8), a variety of both conservative and non-conservative changes were made to residues in Tf1 proximal to the DNA⋅RNA substrate, and these variants were tested for their ability to support prime editing in HEK293T cells. Five mutations that improved editing efficiency were discovered (FIG. 8D): K118R and S118K are at predicted locations that may improve binding with the RNA template, while I260L, S297Q, and R288Q are at predicted locations that may improve binding with the DNA substrate (FIG. 1F). Combining all five mutations additively improved mammalian editing efficiencies, and the final rationally designed variant of Tf1, rdTf1, showed on average 1.8-fold improvement in editing efficiency over wild-type Tf1 in HEK293T cells across seven different prime edits at five genomic loci (FIG. 1G, FIG. 8E).

Structure-guided engineering was also used to improve the editing efficiency of the Ec48 retron RT prime editor. The Ec48 retron RT is even smaller than the Tf1 RT but supports only very low prime editing activity (FIG. 1C). Since the structure of a retron RT³² had not been reported when these experiments were begun, AlphaFold2³³ was used to predict the three-dimensional structure of Ec48 RT (FIG. 8F). The predicted structure aligned well with the RT from the xenotropic murine leukemia virus-related virus (XMRV, PDB: 4HKQ), a close relative of the M-MLV RT³⁴. Indeed, incorporation of T189N in Ec48, the mutation predicted by the AlphaFold2 structure to be analogous to D200N in PE2, improved PE efficiency of the Ec48 RT prime editor by 3-fold on average across six different edits in HEK293T cells (FIGS. 8G, 8H). Aligning the structure of the Ec48 RT with the substrate of the XMRV RT allowed for the identification of five additional mutations that improved PE efficiencies (FIG. 8H): K307R and R378K are predicted to improve RNA substrate binding, L182N and T385R are predicted to improve DNA substrate binding, and R378K is predicted to improve binding to both the RNA and DNA (FIG. 8I). Combining the top-performing three mutations yielded the best Ec48 variant, rdEc48, which exhibits an 8.6-fold improvement in average prime editing efficiency over wild-type Ec48 across six different edits in HEK293T cells (FIGS. 1H, 8J).

Despite these substantial improvements, prime editing efficiencies of all engineered RT enzymes remained lower than PE2 (FIG. 1I). None of the engineered retroviral RT enzymes outperformed PE2, and the most compact engineered RT (rdEc48) still exhibited large deficiencies compared to PE2, yielding 8-fold lower average editing efficiencies (FIG. 1I). Although the engineered RT rdTf1 came close to PE2 levels of editing (93% of PE2 editing) for several edits noted in FIG. 1I, rdTf1 still struggled with longer, more complex edits and performed 1.6-fold worse than PE2 at the same three sites tested in FIG. 1D (FIG. 1J).

Collectively, these data show that rational engineering can substantially improve the PE activity of diverse RT enzymes. However, engineering alone was not sufficient to produce (1) compact RTs that match state-of-the-art prime editing efficiencies, (2) dual-AAV compatible RTs that can catalyze long and difficult edits, or (3) prime editor variants that improve editing efficiency over PE2 or PEmax. To solve these problems, continuous laboratory protein evolution was used.

### Development and Validation of a Prime Editing PACE Selection Circuit

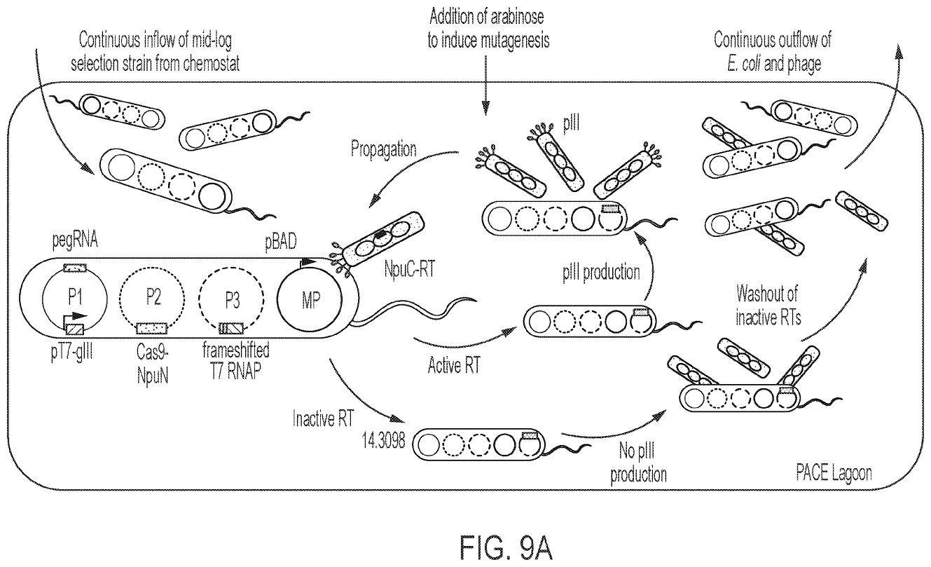

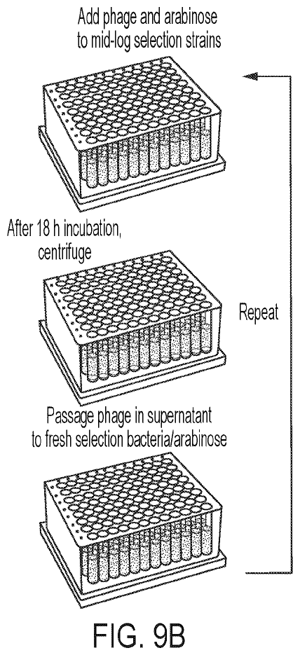

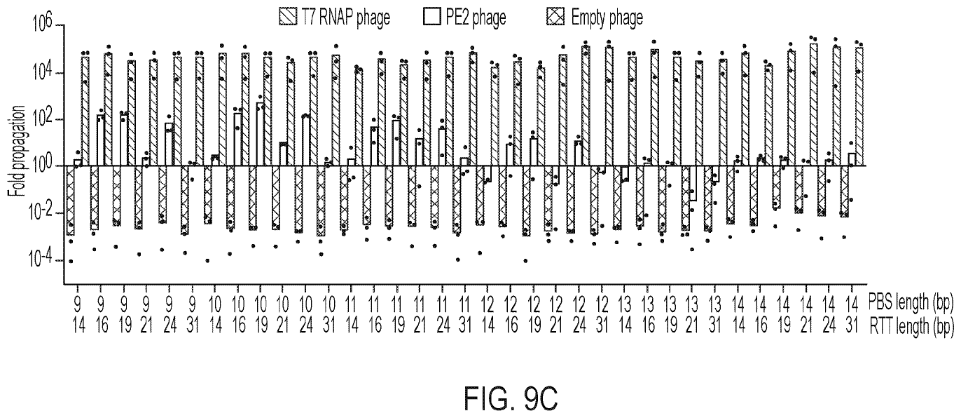

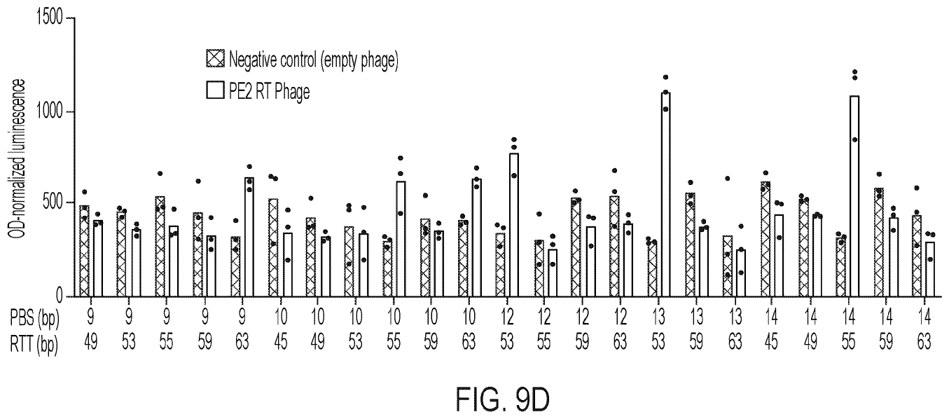

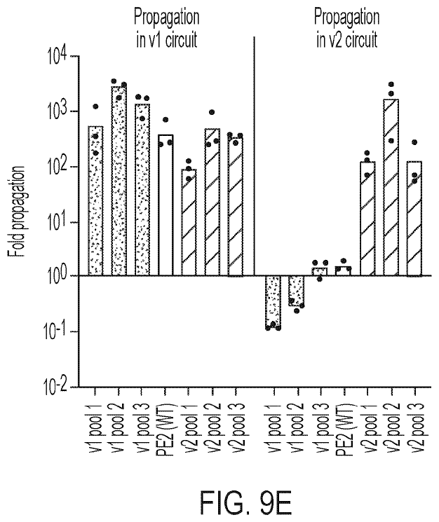

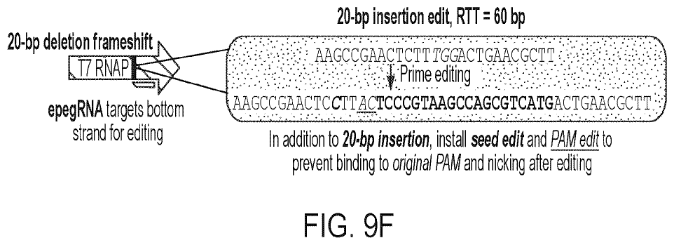

Phage-assisted continuous and non-continuous evolution (PACE and PANCE)^{26,35} are methods for highly accelerated laboratory evolution in which the propagation of a modified M13 bacteriophage is linked to the activity of a protein of interest (FIG. 9A, FIG. 9B). To achieve this linkage, gIII, a gene required for phage propagation, is moved from the phage genome to a plasmid in host E. coli cells under the control of a gene circuit, such that gIII expression and phage propagation are only possible if the phage contain gene(s) that encode proteins with the desired activity. Simultaneous expression of mutagenic proteins from the inducible mutagenesis plasmid MP6 mutagenizes the phage, including the gene of interest³⁶. During PACE, continuous dilution of a fixed-volume “lagoon” with fresh host cells selects for rapidly propagating phage encoding molecules that trigger gIII expression (FIG. 9A). PANCE uses the same selection strategy, but is implemented using discrete dilution steps every 12-24 hours to enrich for phage with increased fitness (FIG. 9B)³⁵. PANCE offers higher sensitivity (lower stringency) and greater ease of parallelization than PACE, with the trade-off of slower evolution. Both methods can complete dozens of generations of gene mutation, selection, and replication every 24 hours.

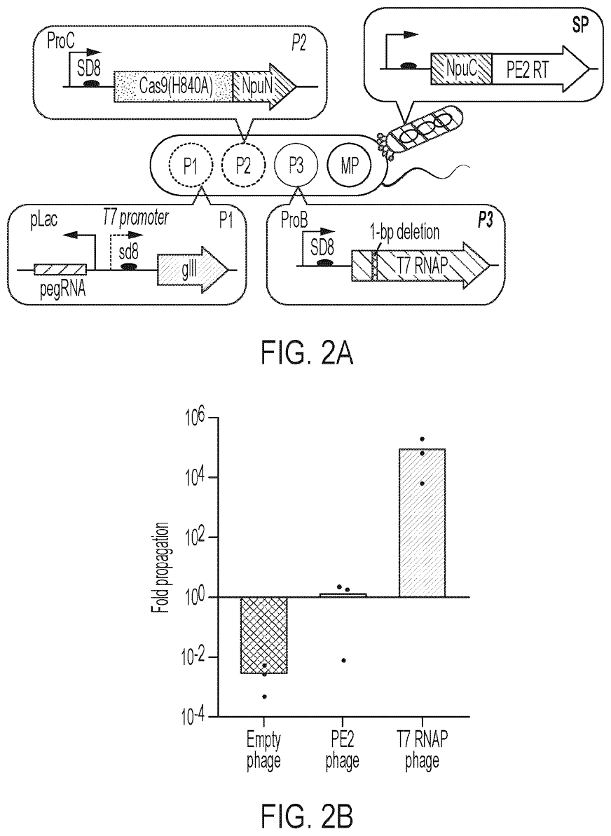

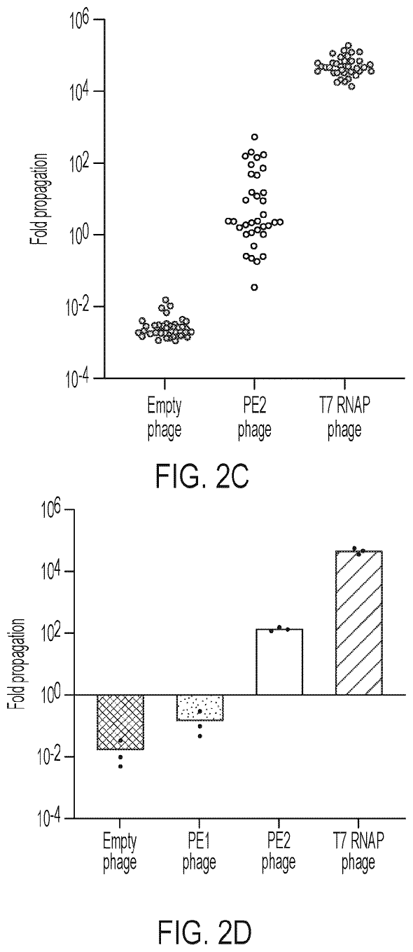

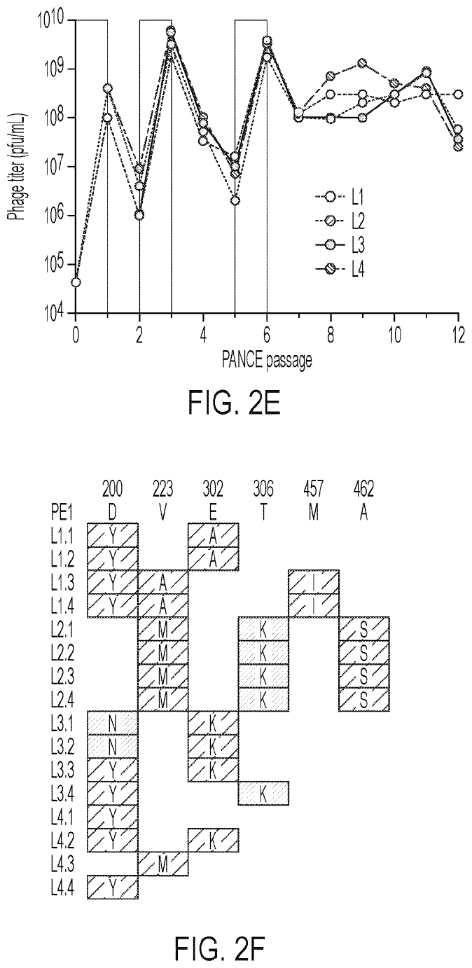

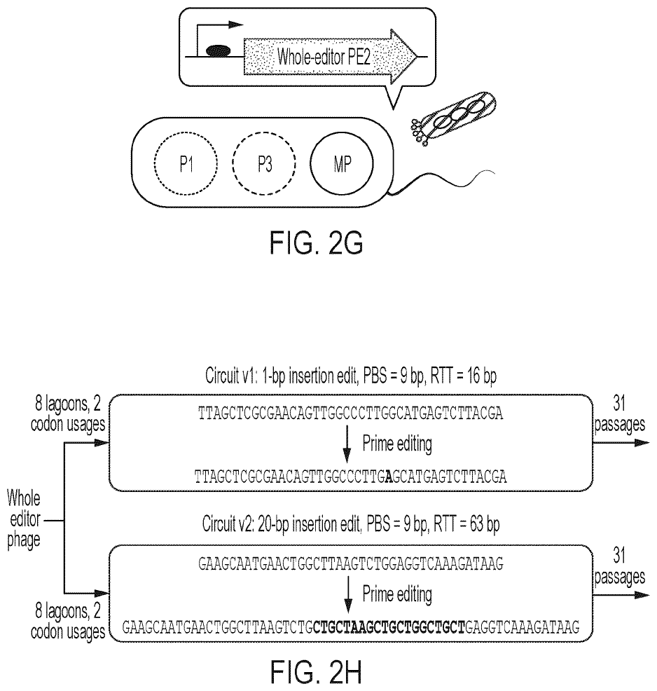

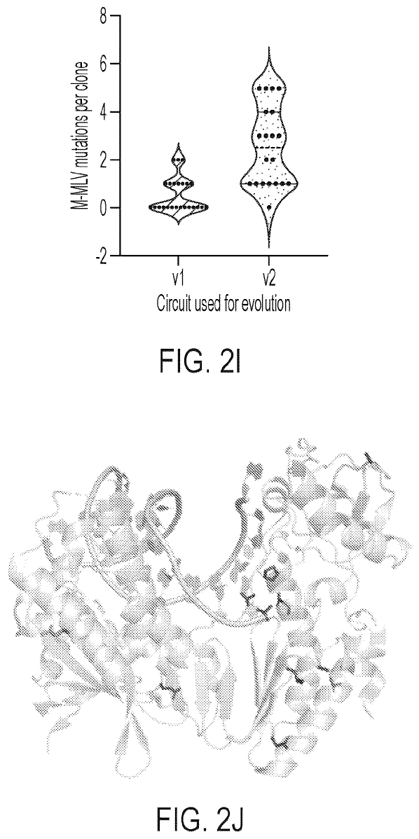

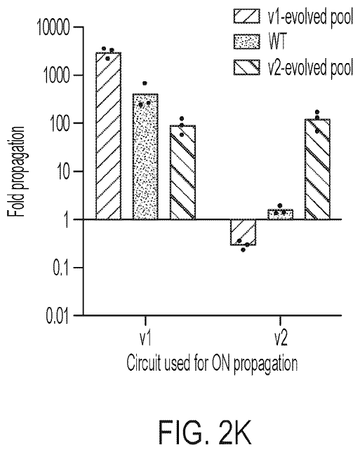

To develop a prime editor PACE (PE PACE) circuit that links PE activity with phage propagation, gIII was removed from the phage genome and placed it under the control of a T7 promoter on a plasmid (P1) in host E. coli. A second plasmid (P2) contained a defective T7 RNA polymerase (T7 RNAP) gene with a 1-bp deletion frameshift mutation. Correction of this frameshift by prime editing enables T7 RNAP production, gIII expression, and phage propagation. In the initial version of the circuit (v1 circuit), various prime editing components were distributed between the host E. coli and the selection phage. SpCas9(H840A) nickase was fused to the N-terminal half of the Npu intein (NpuN) and included on a final plasmid, P3. A C-terminal Npu intein (NpuC) fused to the PE2 RT was encoded on the selection phage, which would allow intein splicing to reconstitute full-length prime editor after phage infection. Finally, a pegRNA encoding the corrective T7 edit was included on P1. This selection design allows the RT, but not the Cas9 nickase domain of the prime editor, to evolve during PACE (FIG. 2A).

This selection circuit was evaluated by overnight phage propagation assays. Empty phage lacking a prime editor strongly de-enriched in the circuit, and phage encoding wild-type T7 RNAP propagated robustly. Initially, though, NpuC-PE2-RT phage only propagated 1.4-fold overnight, indicating the need to optimize the circuit (FIG. 2B). Because prime editing efficiency in mammalian cells is heavily influenced by the PBS and RTT lengths of the pegRNA³⁷, it was anticipated that pegRNA optimization would also be important for the PACE circuit. A matrix of PBS and RTT lengths was therefore tested for a total of 35 pegRNAs, and it was found that propagation of NpuC-PE2-RT phage varied 14,000-fold depending on the pegRNA (FIG. 2C, FIG. 9C). These results underscore the importance of pegRNA optimization for prime editing and enabled robust (over 100-fold) overnight propagation of NpuC-PE2-RT phage when an effective pegRNA was used.

Next, it was confirmed that phage propagation correlated with prime editing efficiency. Because the five engineered RT mutations in PE2 improve prime editing efficiencies typically by ~7-fold compared with PE1¹, PE1 served as a useful probe for assessing the dynamic range of the selection circuit. NpuC-PE1-RT phage, were generated and evaluated in the pegRNA-optimized circuit, and it was found that NpuC-PE1-RT phage de-enriched 6.7-fold, while NpuC-PE2-RT phage propagated 140-fold (FIG. 2D), establishing that the selection can distinguish prime editor RT variants based on their ability to support prime editing. Finally, to verify that this circuit can enrich mutations that enhance prime editing, NpuC-PE1-RT phage were evolved in PANCE. After eight overnight PANCE passages with 1:50 dilution, phage titers began to stabilize (FIG. 2E), and sequencing of individual surviving phage revealed the convergence of several mutations (FIG. 2F). Encouragingly, two of the six mutations that converged in PANCE are also found in PE2, demonstrating that PANCE of an RT can evolve mutations known to enhance prime editing in mammalian cells.

### High-Stringency PE PACE Reveals Edit-Dependent Effects on Evolved Editors

Next, a means for increasing selection stringency during PE PACE was determined. To date, edit-dependent effects on prime editing efficiency are thought to result from features of the target site and downstream DNA repair, as opposed to preferences of the prime editor protein itself^{3,38-41}. Based on the observation that RTs such as PE2ΔRNaseH and rdTf1 were more efficient for small edits using short RTTs but showed deficiencies when using long RTTs (FIG. 1C and FIG. 1D), it was hypothesized that increasing edit size and RTT length would increase the stringency of the PE PACE circuit compared to the original v1 circuit. A second circuit was designed and optimized (v2, FIG. 9D) in which a 20-bp insertion, instead of the 1-bp insertion used in the v1 circuit, is required to enable phage propagation.

It was also believed that evolving RTs in the context of a complete fusion to Cas9 nickase, as opposed to a split-intein architecture that focuses evolution only on the RT domain, would better reflect their eventual use as prime editors, while also allowing for the evolution of Cas9 nickase domain variants that might enhance prime editing outcomes. To create this alternate architecture, the P2 plasmid was removed from the host E. coli and the entire prime editor protein, including the Cas9 nickase domain, was encoded on the phage without the use of a host P2 plasmid or split inteins (FIG. 2G).

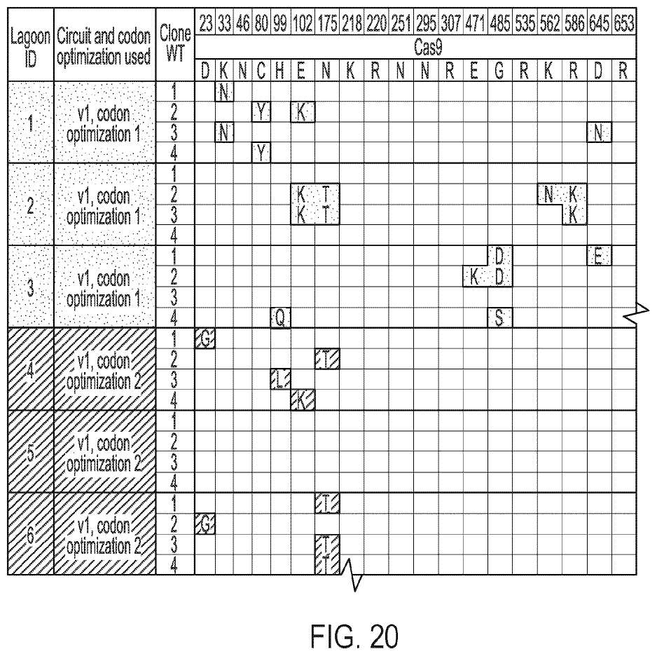

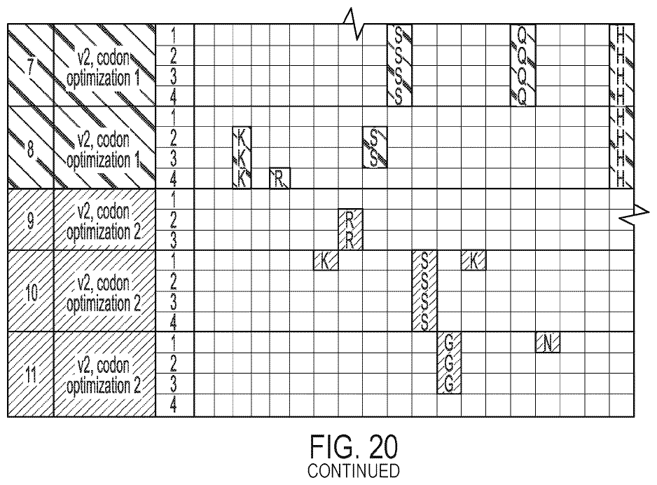

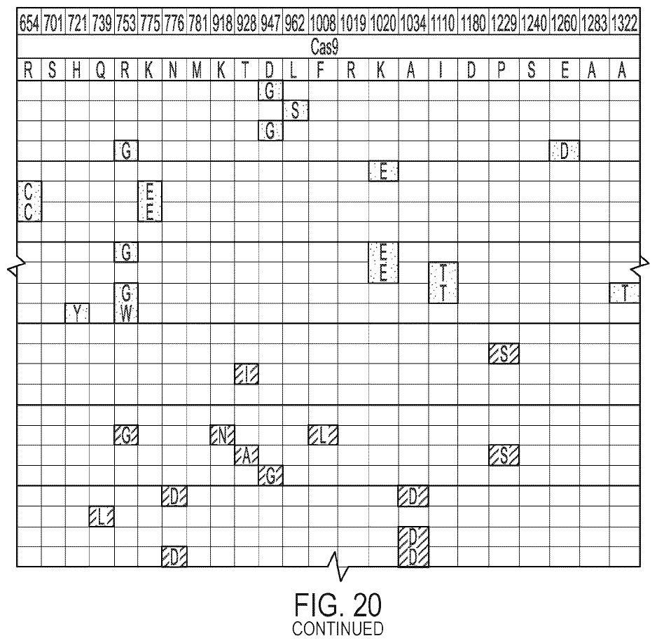

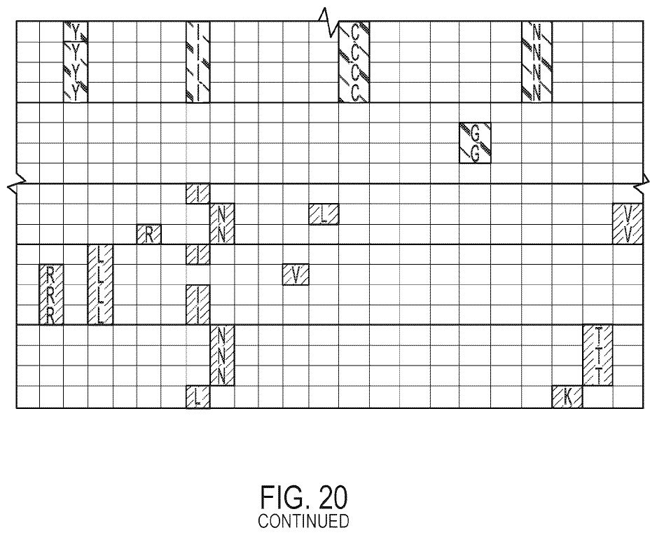

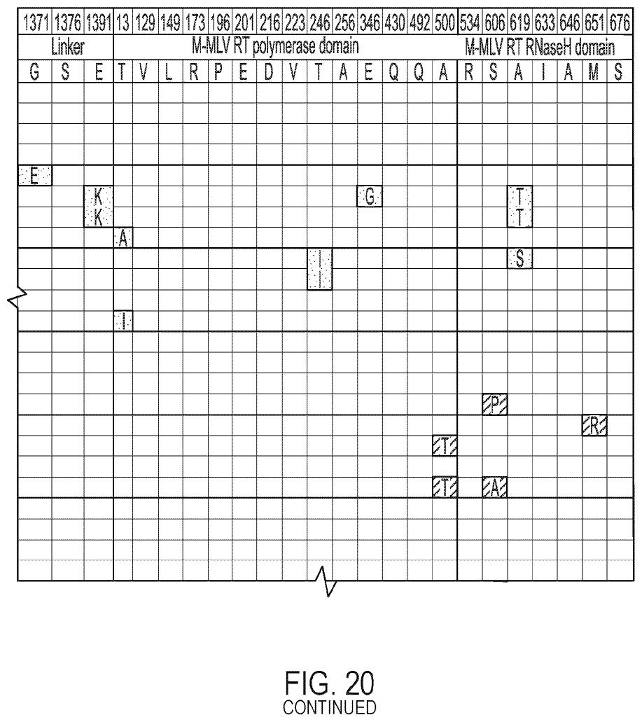

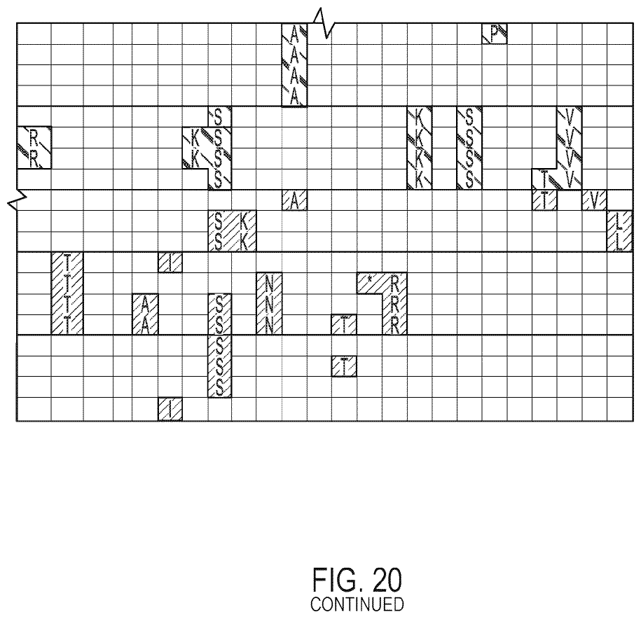

To study the effects of these circuit changes, a comparative PANCE experiment evolving the same whole-editor PE2 phage was designed using the v1 or v2 circuit to study the effects of the target edit on evolution outcomes (FIG. 2H). To account for the fact that stochastic mutational differences can cause different evolutionary outcomes even under identical selection conditions⁴², eight replicates of each PANCE condition were performed, using two different codon optimizations to diversify available trajectories on the editor's fitness landscape. After 31 passages of PANCE, clonal phage from six different v1 lagoons and five different v2 lagoons were sequenced (FIG. 20, FIG. 2H). While convergent mutations were consistent between PANCE replicates within a given edit, conserved mutations differed greatly between lagoons that were required to perform the two different edits. On average, the 20-bp insertion selection generated more RT mutations than the 1-bp insertion selection (FIG. 2I), consistent with the hypothesis that selections requiring longer RTTs exert more evolutionary pressure on the reverse transcriptase. Mutations evolved in the v2 circuit were also located closer to the polymerase's active site, whereas residues evolved in the v1 circuit were typically surface-exposed (FIG. 2J). These findings demonstrated that the target edit used in PE PACE strongly affects the resulting genotypes, suggesting that the most efficient prime editors may specialize in specific types of edits.

To investigate this possibility, pools of phage evolved in the 1-bp insertion vs 20-bp insertion PANCE experiment were used, and overnight propagation studies were performed on either the matched or mismatched evolution strain. It was observed that when phage were evaluated in the strain in which they were evolved, their propagation improved relative to starting whole-editor PE2 phage. However, when evolved phage were evaluated in a strain requiring the other edit, they propagated less well than the parental PE2 phage. For example, v1-evolved phage (requiring a 1-bp insertion) showed increased propagation in the v1 strain but decreased propagation in the v2 strain (requiring a 20-bp insertion) and vice versa (FIG. 2K, FIG. 9E). Although it was anticipated that prime editors evolved on long-RTT edits would also improve outcomes for short-RTT edits, the data instead indicated that prime editors enriched by the different edits in the v1 and v2 circuits evolved properties that specialize in their respective edits, and thus different prime editors will likely be best for different types of edits.

The above insights, as well as other recent PE improvements, were combined to design a v3 PE PACE circuit. This final version used engineered pegRNAs (epegRNAs)⁵, which broadly improve prime editing by protecting pegRNAs from cellular degradation, to correct a different 20-bp deletion in T7 RNAP (FIG. 9F). The v1-v3 PE PACE circuits were then used to evolve several different RTs.

### Evolution of Compact RTs

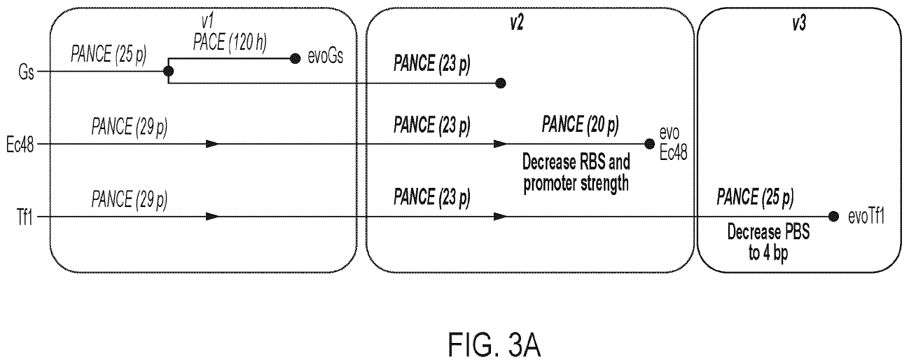

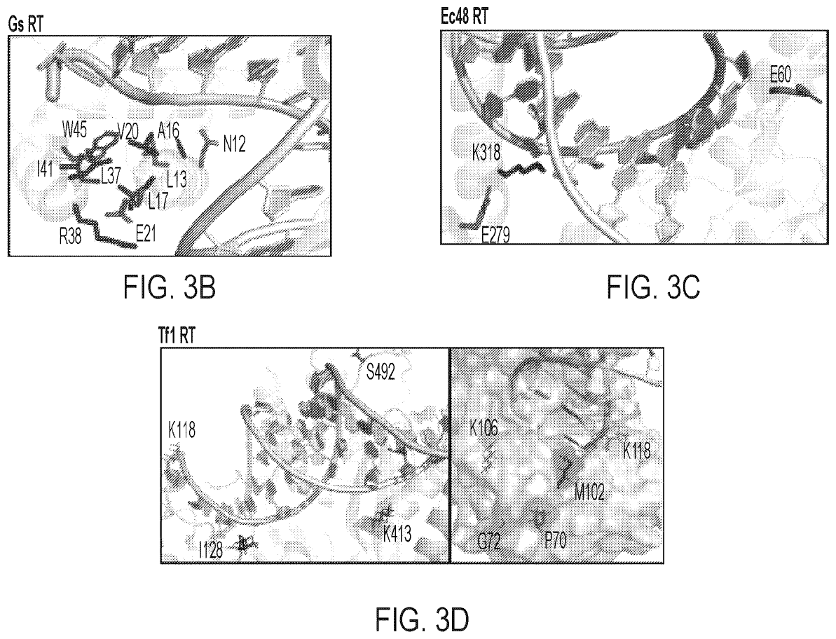

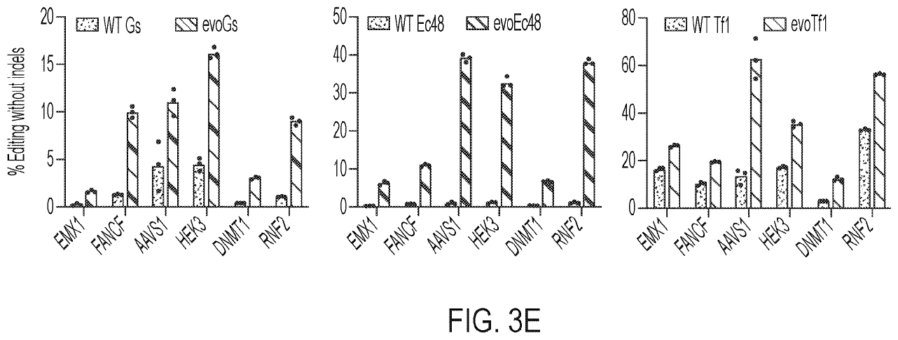

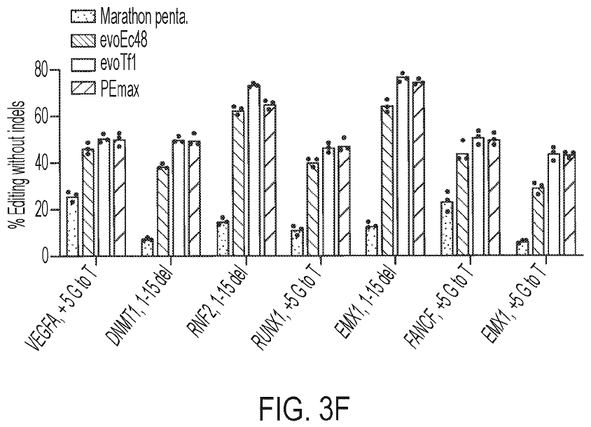

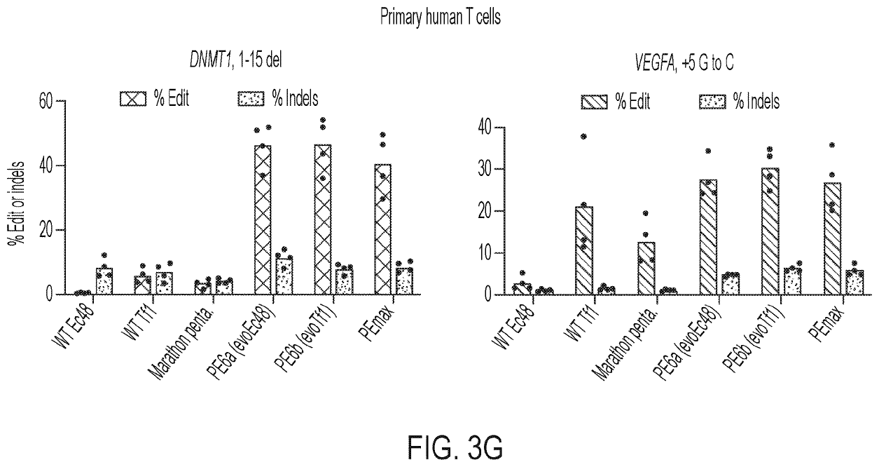

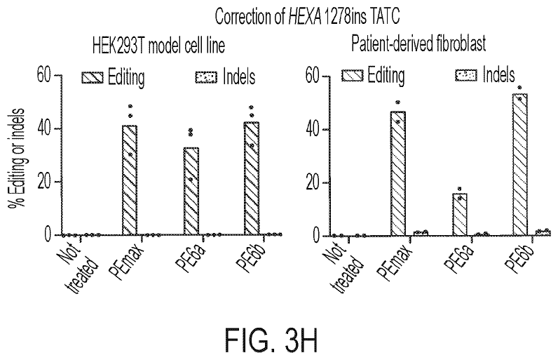

PE PACE was first used to evolve three compact RTs identified from the initial screen (FIG. 1C), focusing on RTs that are substantially smaller than the PE2 RT. This included the Geobacillus stearothermophilus GsI-IIC intron RT (Gs RT), as well as the Ec48 and Tf1 RTs engineered above. The various evolutionary trajectories pursued are summarized below and in FIG. 3A.

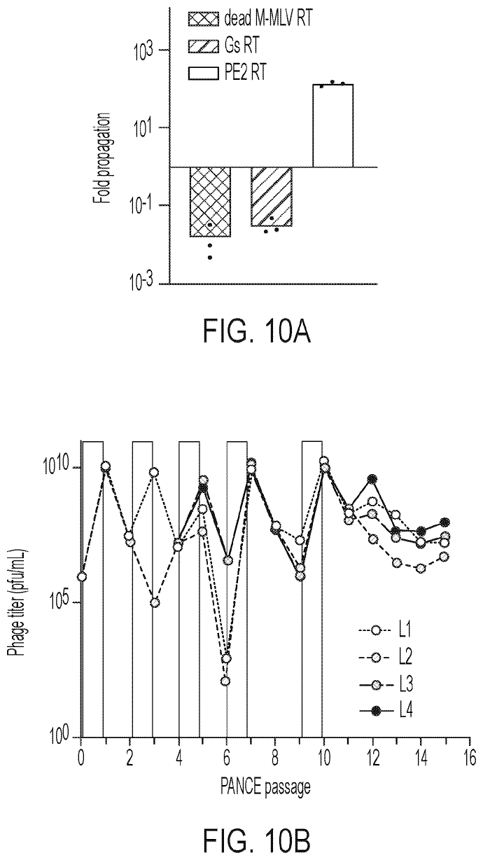

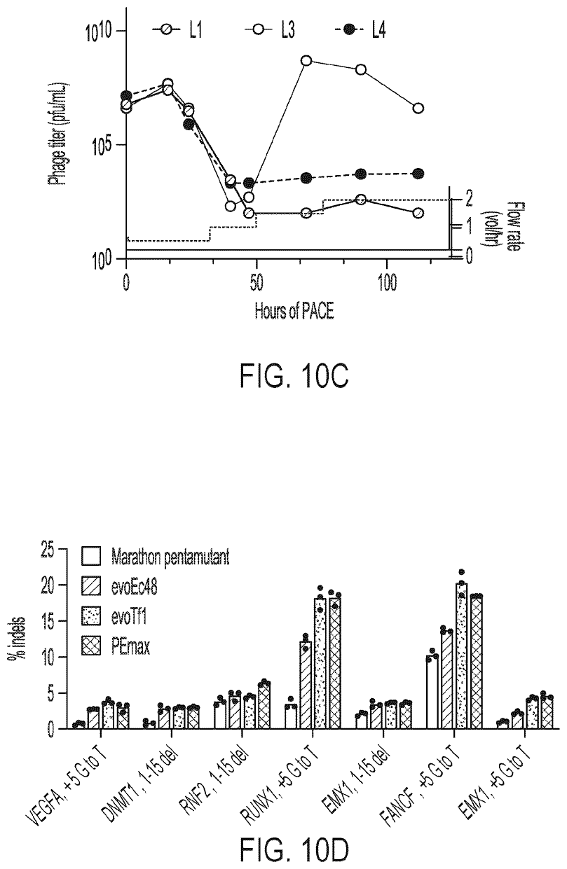

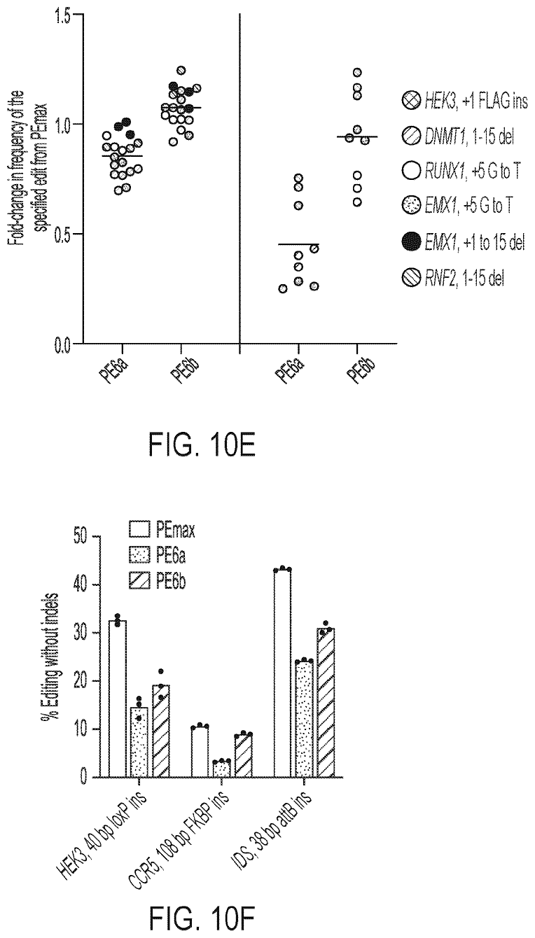

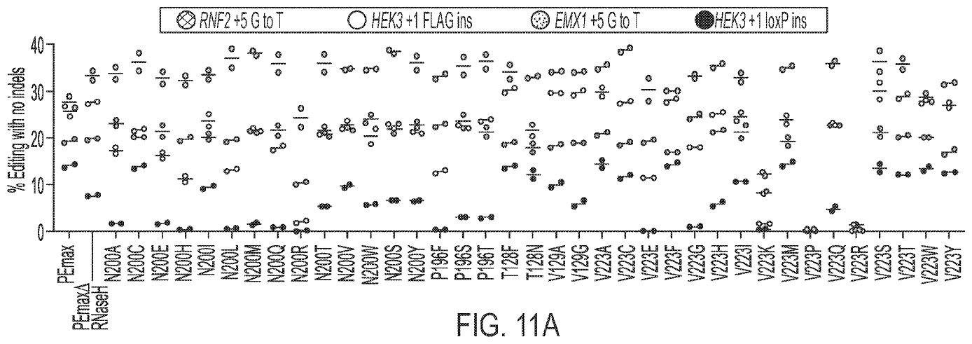

The starting point was Gs RT, since its origin from a thermophilic organism may offer greater folding stability⁴³, it has been used for in vitro cDNA generation⁴⁴, and it had not previously been engineered during the rational engineering efforts described in FIGS. 1A-1J. Wild-type Gs RT supports minimal prime editing in mammalian cells (FIG. 1C), and phage encoding NpuC-Gs-RT did not show any activity above negative controls in overnight propagation assays (FIG. 10A). Therefore, low-stringency PANCE was performed in the split intein v1 circuit with frequent passages under no selection pressure (drift) to restore phage titers; after twelve passages, phage began to propagate more robustly under selection (FIG. 10B). These pools of evolved phage were then used to seed a 100-hour long PACE experiment (FIG. 10C). Simultaneously, phage pools obtained from PANCE v1 were also used to seed a second PANCE using the more stringent v2 circuit described above. This PANCE was performed for 23 additional passages.

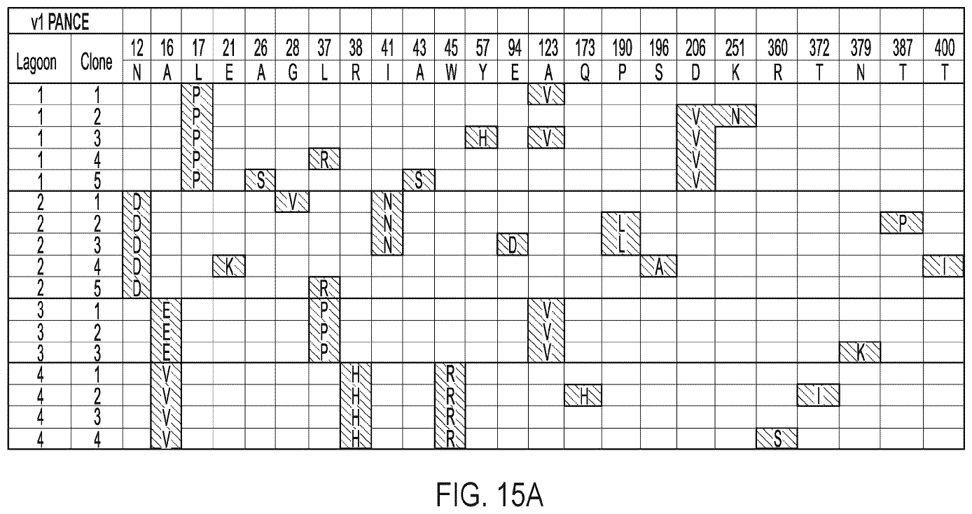

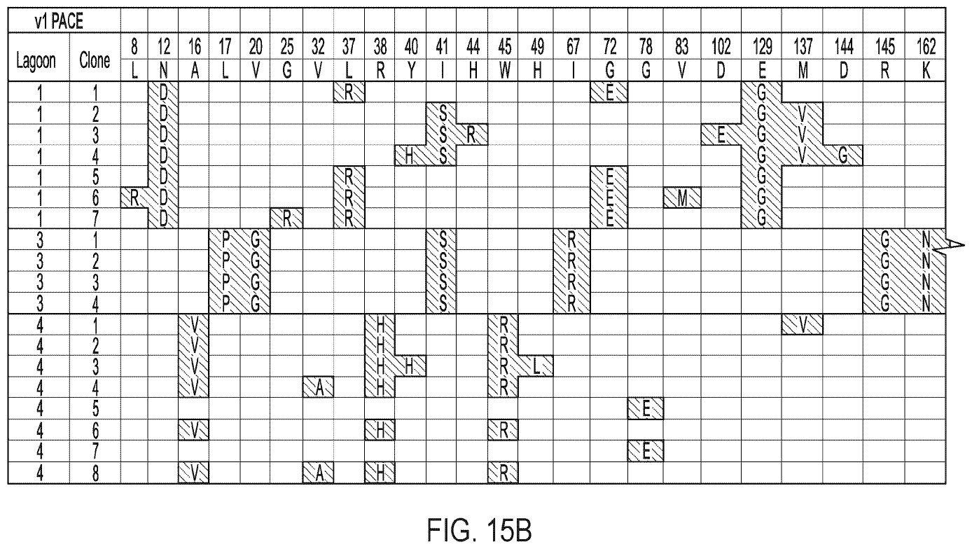

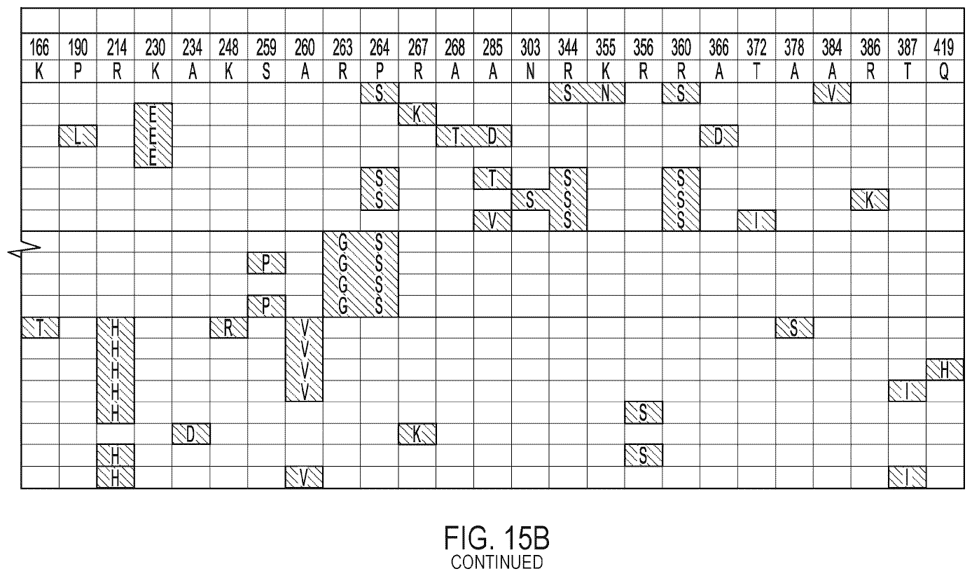

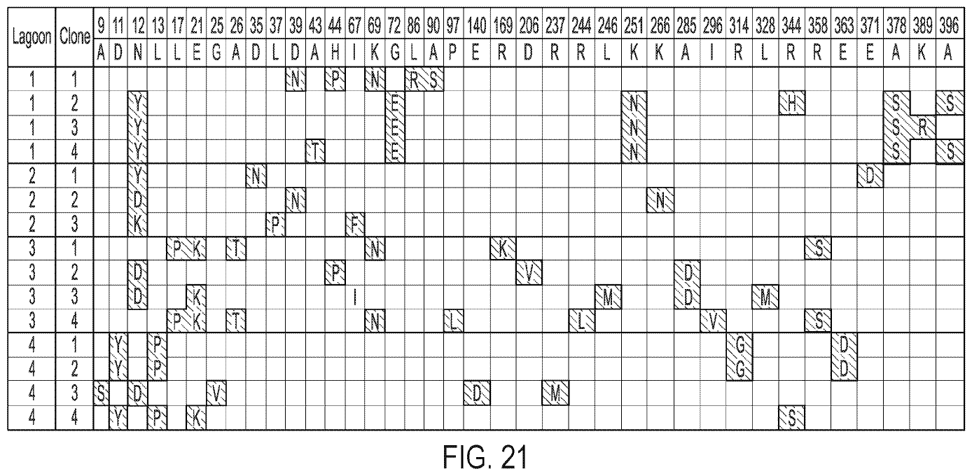

Sequencing the Gs RT phage from different evolutions and mapping these mutations onto the crystal structure of wild-type Gs RT (PDB: 6AR1)⁴³ revealed a high degree of predicted structural convergence among the mutated residues (FIGS. 15A-15B, FIG. 21). Each evolved clone harbored mutations (such as N12D, A16E/V, L17P, L37P/R, R38H, 141N/S, and W45R) that appear to perturb the interaction between two alpha helices of the Gs RT's N-terminal extension (NTE) (FIG. 3B). One of these alpha helices protrudes directly into the major groove of the DNA/RNA duplex substrate, suggesting that these mutations may improve substrate binding.

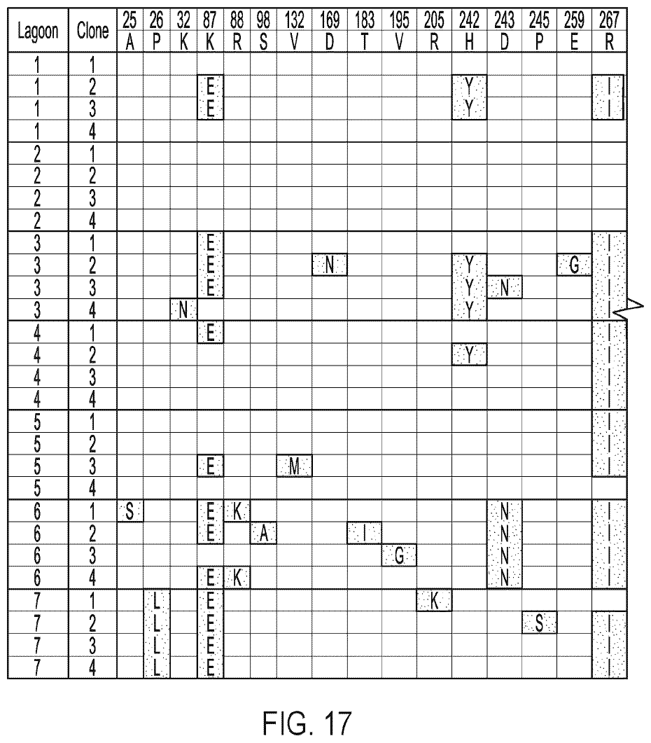

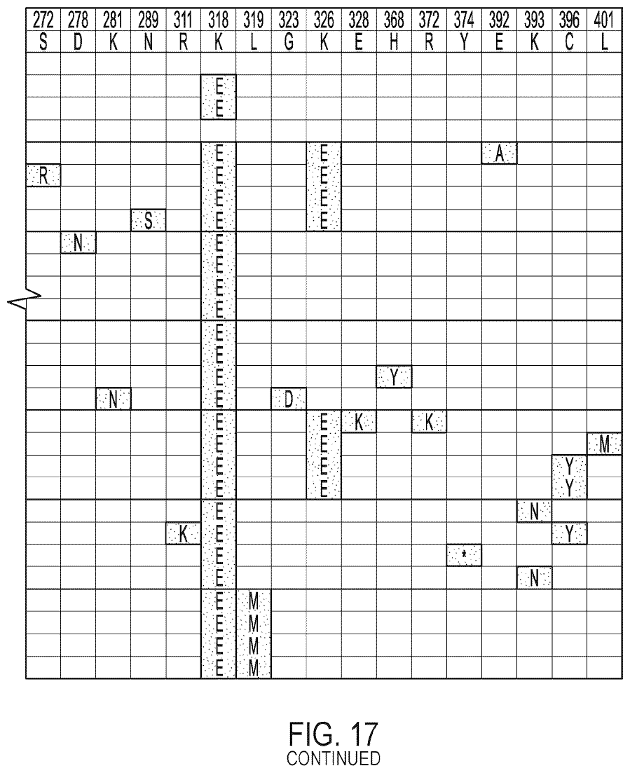

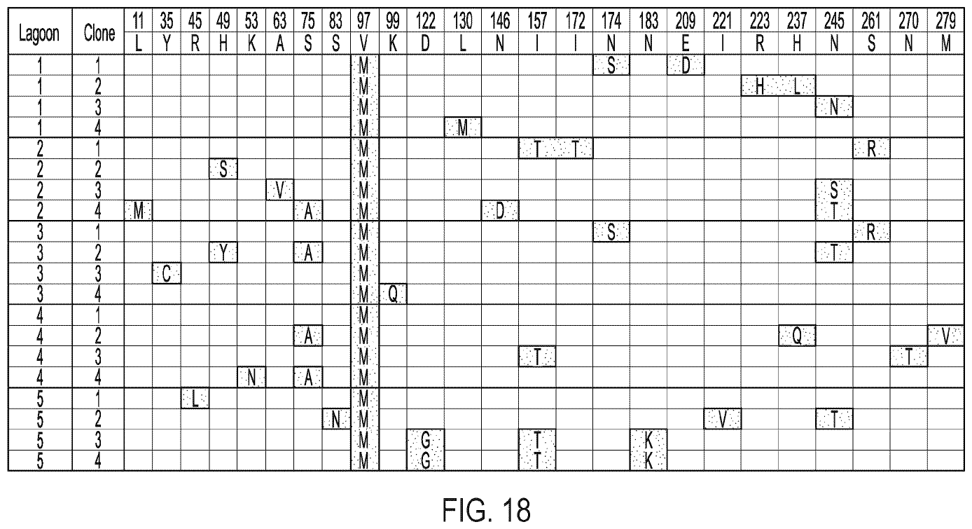

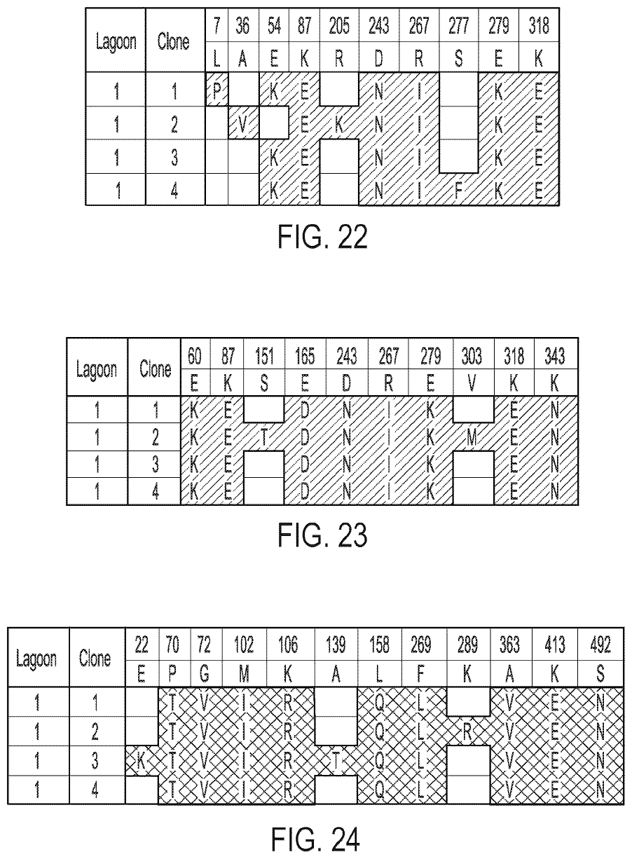

A similar approach was used to evolve the compact Ec48 RT (FIG. 1C). Ec48 RT phage was evolved using PANCE in the v1 circuit for 29 passages and then for 23 passages in the v2 circuit. The stringency of the v2 circuit was then increased by decreasing the RBS and promoter strength used to express T7 RNAP, and the phage was further evolved for another 20 passages. Sequencing of evolved phage pools revealed high levels of convergence (FIGS. 17, 22, and 23). Three residues from the evolution that altered their charge (E60K, E279K, and K318E) are in close proximity to the DNA⋅RNA substrate (FIG. 3C) in the AlphaFold-predicted structure, suggesting that they also may alter substrate binding.

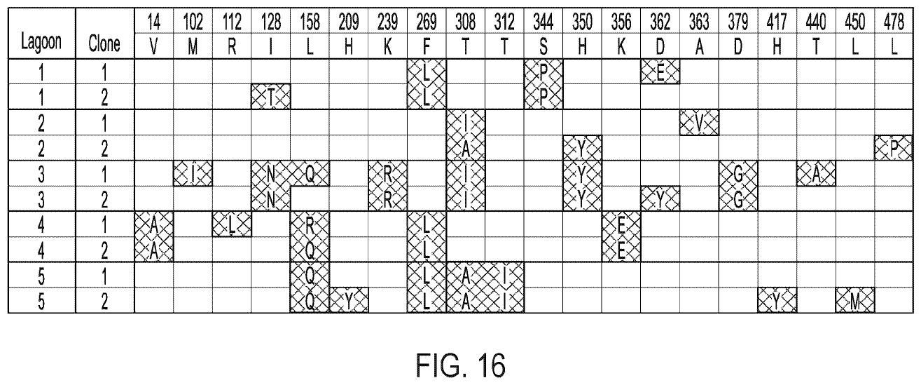

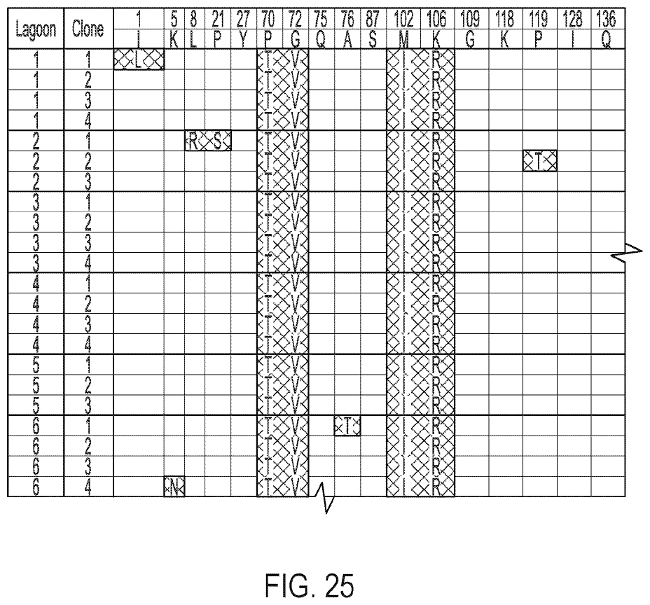

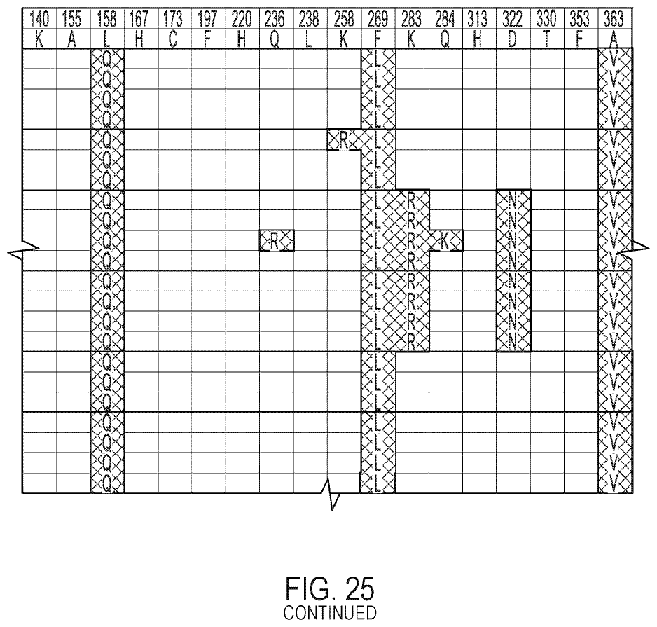

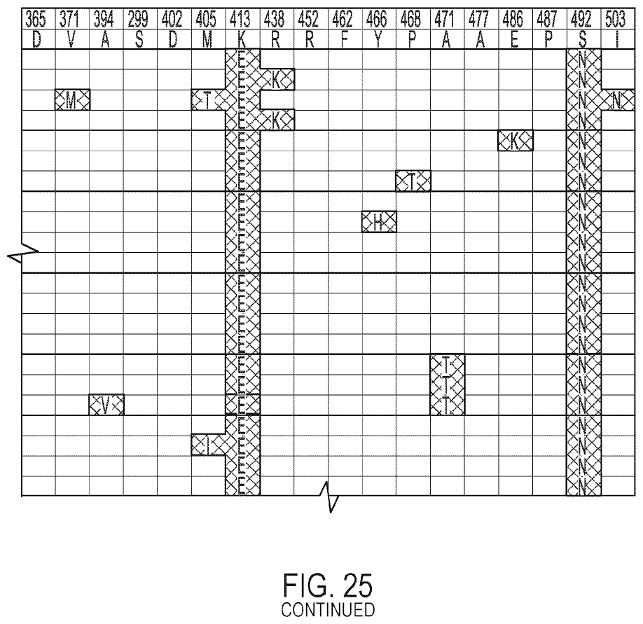

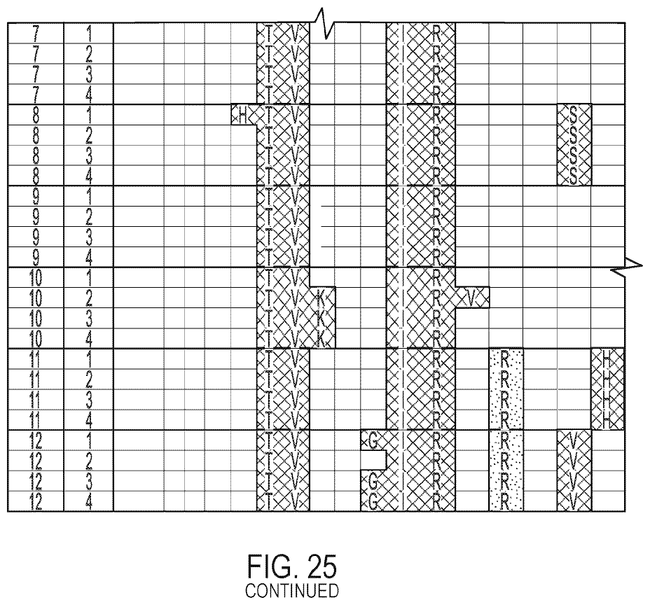

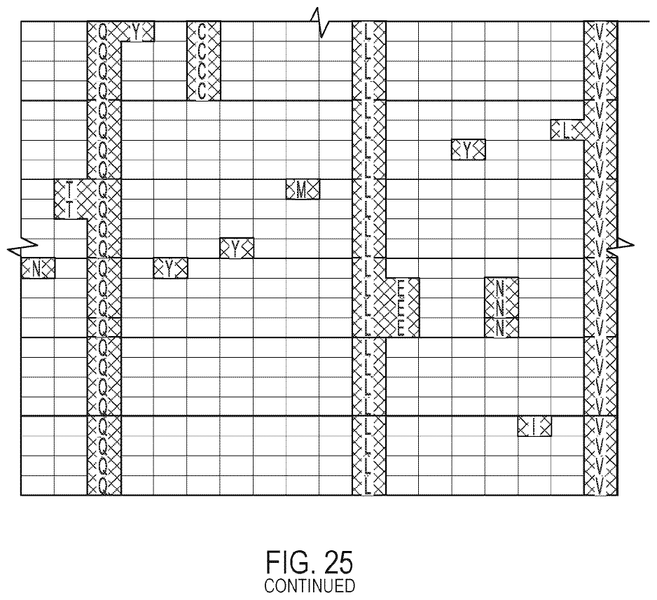

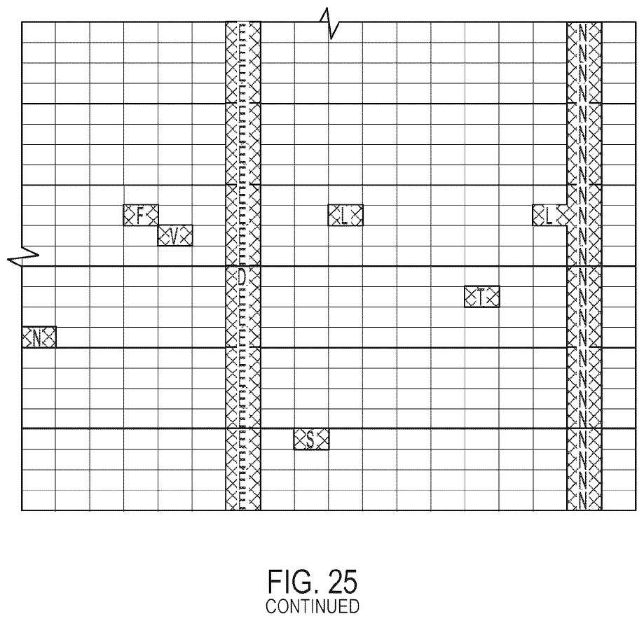

Finally, PANCE was used to evolve the Tf1 RT in all three versions of the circuit. Tf1 RT phage survived 29 passages in the v1 circuit, then 23 passages in the v2 circuit, and finally 25 passages in the v3 circuit. In the v3 circuit, selection stringency was continuously increased by decreasing the PBS length used for evolution from 7 nt to just 4 nt. Several of the resulting converged mutations (K118R, I128V, K413E, and S492N) are in close proximity to the DNA⋅RNA duplex in the AlphaFold predicted structure of the enzyme, while several others (P70T, G72V, M102I, and K106R) decorate the surface of the enzyme that may interact with the RTT of the pegRNA (FIG. 3D; FIGS. 16, 24, and 25). Previously in the structure-based engineering efforts, K118R was identified as the most beneficial mutation for Tf1 RT prime editing in HEK293T cells (FIG. 1E). The emergence of this same mutation was observed in two separate lagoons after evolution (FIG. 25), suggesting that the evolutions selected for mutations that improve prime editing efficiencies. Collectively, these data demonstrate that PE-PANCE enables the rapid, parallel evolution of prime editors and is generalizable to diverse RTs.

### Mammalian Cell Characterization of Compact Evolved RTs

Following these evolution campaigns, evolved RT domains were cloned for Gs RT, Ec48 RT, and Tf1 RT into mammalian expression cassettes, and their performance was evaluated in HEK293Tcells. First, evo-RT variants were cloned as PE2 proteins and compared to the corresponding WT enzymes across six different edits (FIG. 3E). Evolved RT variants greatly outperformed their WT counterparts; a 6.2-fold improvement for evo-Gs, a 22-fold improvement for evo-Ec48, and a 2.7-fold improvement for evo-Tf1 was observed. These results demonstrate that PE PACE can evolve substantial improvements in mammalian prime editing activity for many different types of RT enzymes.

Of these RTs, evo-Tf1 offered the highest average editing efficiency, and evo-Ec48 was the most compact RT (1.2 bp). These two enzymes were therefore further characterized in the recently optimized PEmax architecture, which improves codon optimization, linkers, and nuclear localization signals to enhance prime editing efficiency over PE2³. These evolved editors were compared to PEmax (2.2 kb) and PEmaxΔRNaseH (1.5 kb), as well as the Marathon pentamutant RT engineered by Joung and coworkers²³. The Marathon pentamutant, which is 1.2 kb in length, is the current state-of-the-art size-minimized prime editor. These five editors were evaluated at eight edits that all used epegRNAs in HEK293T cells.

First, it was observed that evo-Ec48 substantially outperformed the engineered Marathon pentamutant²³, by 3.7-fold on average. Furthermore, evo-Ec48 yielded comparable editing efficiencies relative to PEmax, generating on average 80% of PEmax editing efficiencies across the eight edits tested (FIG. 3F and FIG. 10D). Since evoEc48 is 810 bp smaller in gene size than the engineered M-MLV RT used in PEmax, 270 bp smaller than the ΔRNaseH form of M-MLV, and more efficient than the size-equivalent Marathon pentamutant, evo-Ec48's use is recommended for prime editing applications in which the size of the prime editor must be minimized. It was also noted that the use of epegRNAs is important for achieving efficient prime editing with evo-Ec48 (FIG. 10E). Moving forward, the evo-Ec48 based prime editor is renamed PE6a.

Next, it was observed that evo-Tf1 on average supported prime editing levels equal to those of PEmax at the eight edits tested (FIG. 3F and FIG. 10D). Moving forward, the evo-Tf1 based prime editor is referred to as PE6b. Both PE6a and PE6b may be less efficient at longer, complex edits (FIG. 10F).

To examine the applicability of PE6a and PE6b variants in a therapeutically relevant cell type, the performance of PE6a, PE6b, their wild-type RT counterparts, the Marathon pentamutant, and PEmax were compared in primary human T cells at the VEGFA and DNMT1 loci following electroporation of the corresponding PE mRNA and pegRNA. For the DNMT1 1-15 del edit, wild-type Ec48 was minimally active (0.22% average editing efficiency), and the Marathon pentamutant yielded 3.3% editing. The similarly-sized PE6a supported 47% average editing, a 211-fold improvement over its wild-type counterpart and a 14-fold improvement over the Marathon pentamutant. PE6a was also able to meet or exceed the editing efficiency of PEmax (110% on average) (FIG. 3G). PE6b also offered large improvements over its wild-type RT counterpart, yielding an 8-fold improvement in editing efficiency over PE using wild-type Tf1 and editing efficiency comparable to that of PEmax (FIG. 3G). For all of these prime editors, similar trends were observed for the VEGFA edit. Therefore, PE6a and PE6b can offer editing efficiencies comparable to those of PEmax (FIG. 3G) in a compact form in T cells.

The performance of PE6a and PE6b were evaluated in a HEK293T cell model that harbors the HEXA 1278insTATC mutation, the most common gene variant that causes Tay-Sachs disease^{1,5}. Treatment of this Tay-Sachs disease cell model with PE6a and PE6b and an epegRNA programmed to delete the pathogenic TATC insertion in HEXA yielded 33% and 42% correction, respectively, of the pathogenic mutation. These values are comparable to the 41% correction generated by PEmax (FIG. 3H). Then, either PE6a, PE6b, or PEmax mRNA were electroporated along with the epegRNA and nicking sgRNA, into Tay-Sachs disease patient-derived fibroblasts harboring the 1278insTATC mutation. All three editors generated a therapeutically relevant level of correction (>2% installation of wild-type HEXA⁴⁵): PEmax yielded 46% correction, PE6b generated an average of 53% correction in patient-derived fibroblasts, and PE6a produced an average correction of 16% (FIG. 3H).

Overall, these findings establish that size-minimized, compact RTs can support state-of-the-art prime editing efficiencies in therapeutically relevant cells. Both PE6a and PE6b, which use novel, non-M-MLV RTs, can match or exceed PEmax's editing efficiencies, while also offering substantially smaller gene sizes (1.2 kb and 1.5 kb for PE6a and PE6b, vs. 2.2 kb for PEmax).

PE6a and PE6b are the first enzymes in a suite of improved PE6 variants (PE6a-g) developed herein. To simplify nomenclature, PE6 variants are defined as mutants made in the prime editor protein in the PEmax architecture background. PE6 variants, PEmax, and PEmaxΔRNaseH will be compared for both PE and twinPE edits. When used for PE, the use of a nicking sgRNA is assumed unless stated otherwise. The use of MLH1dn is not assumed and is specified on a case-by-case basis.

### Evolution and Engineering of Highly Active AAV-Compatible RTs

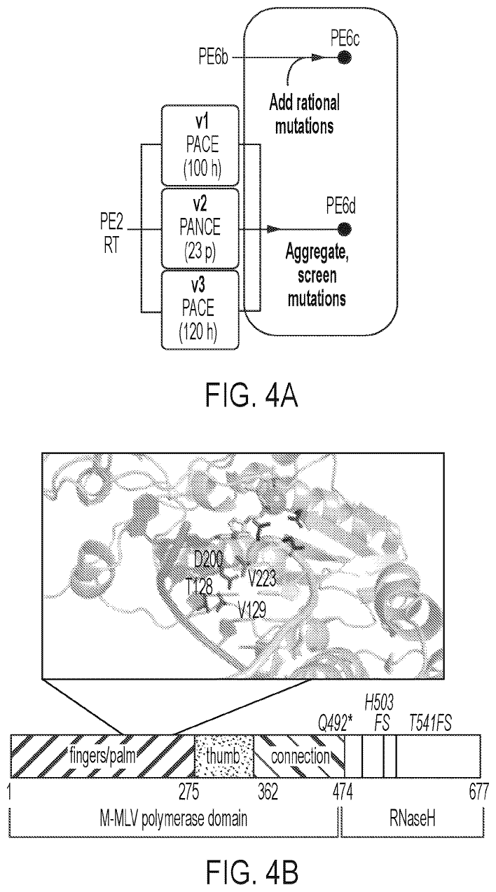

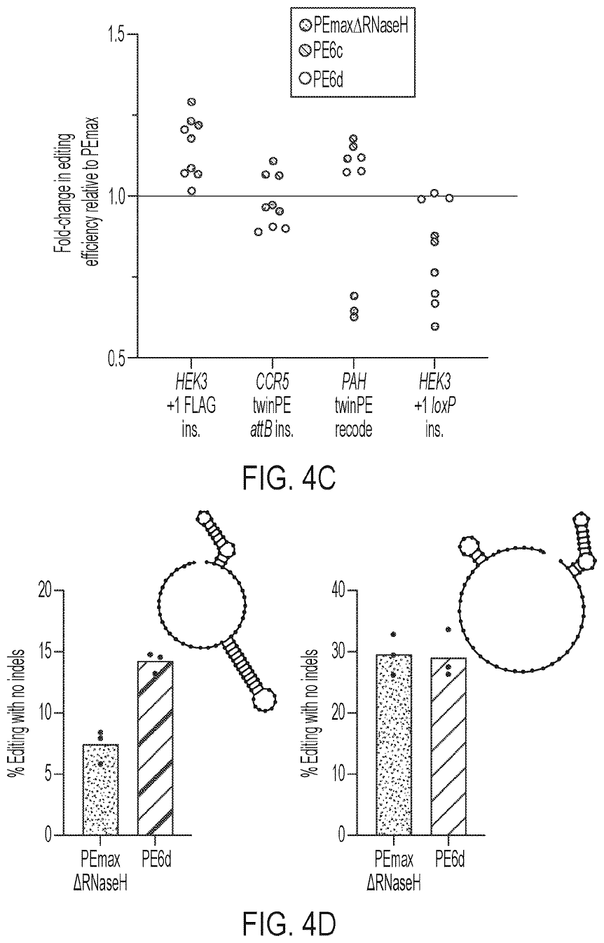

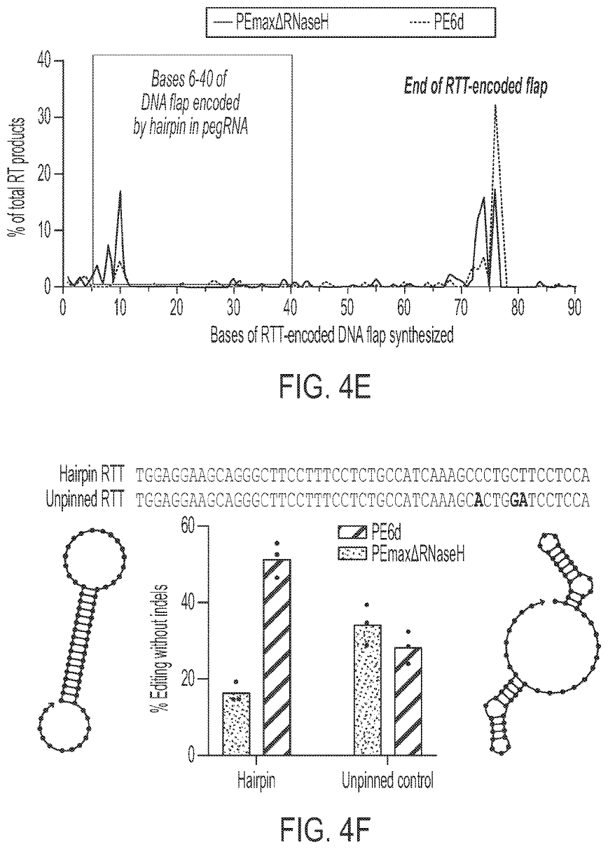

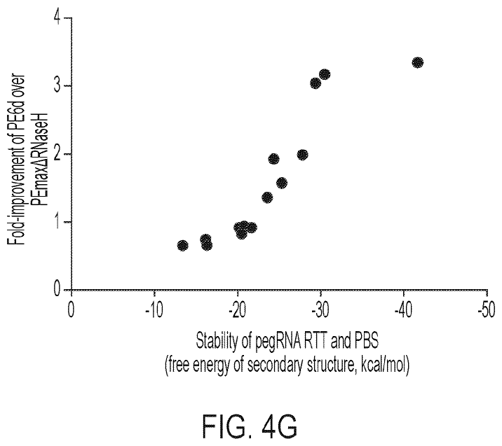

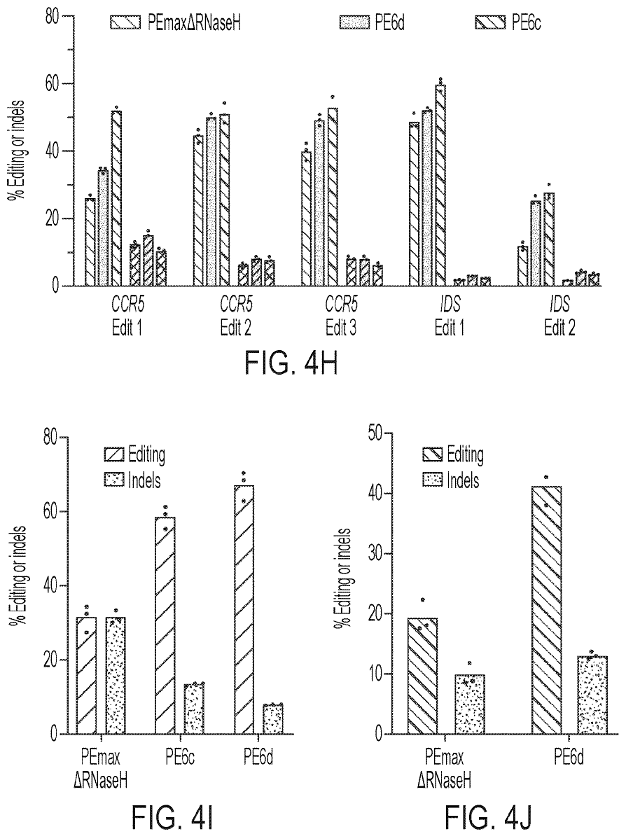

Next, PE PACE was combined with protein engineering to generate prime editors that are the same size as PEmaxΔRNaseH, but offer the ability to make long, complex edits requiring reverse transcription of ten or more nucleotides. This goal was pursued using two different RTs: the Tf1 RT and the M-MLV RT. First, to create a highly active Tf1 RT, mutations in the evolved Tf1 RT (PE6b) were combined with mutations that were originally identified by rationally engineering the enzyme into rdTf1. The resulting engineered and evolved Tf1 was named PE6c (FIG. 4A). PE6c harbors a total of sixteen unique mutations from evolution and rational engineering.

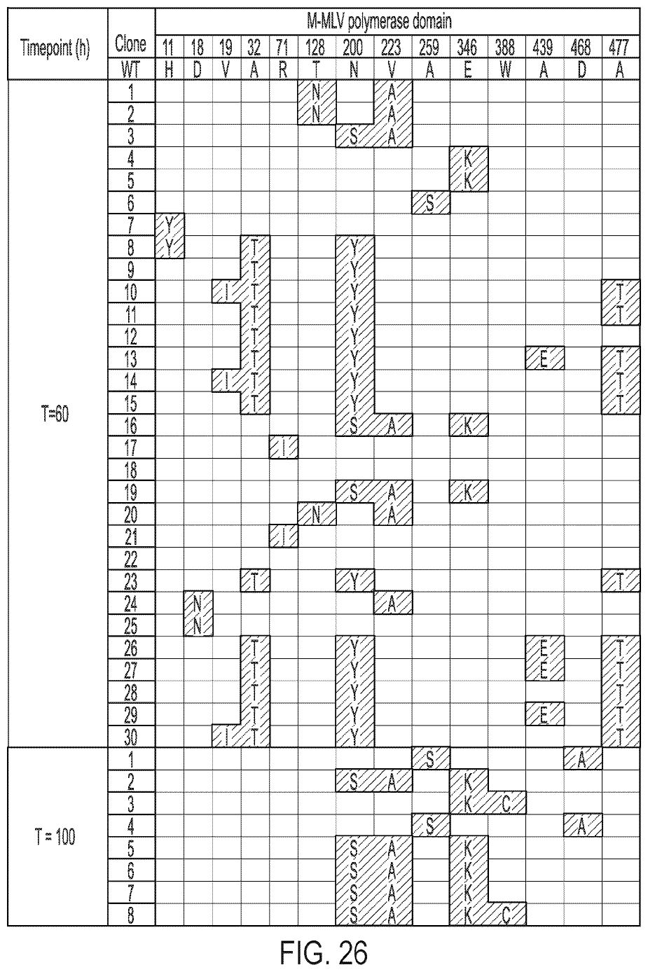

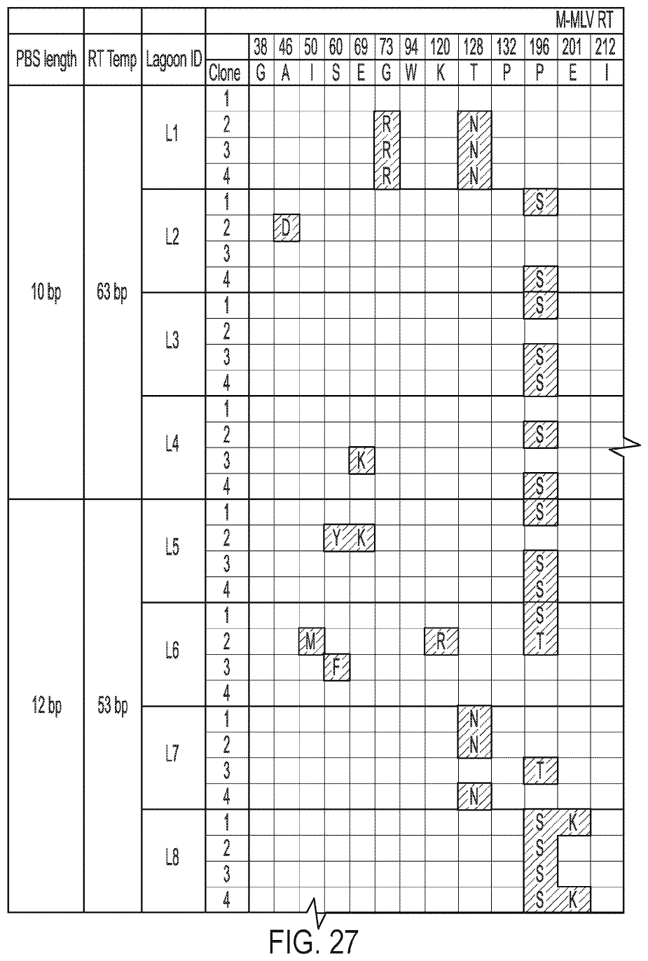

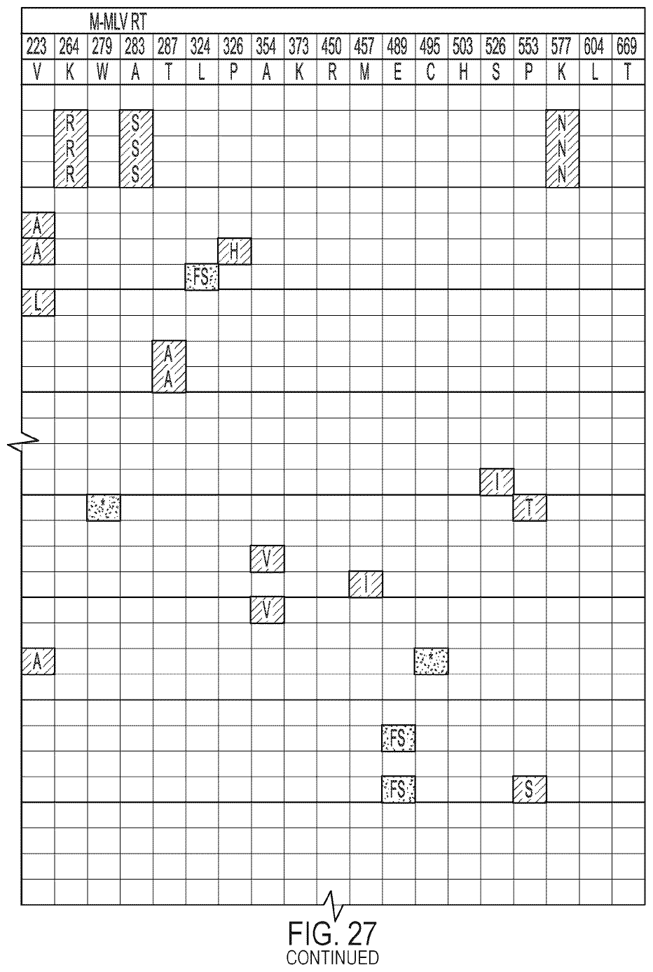

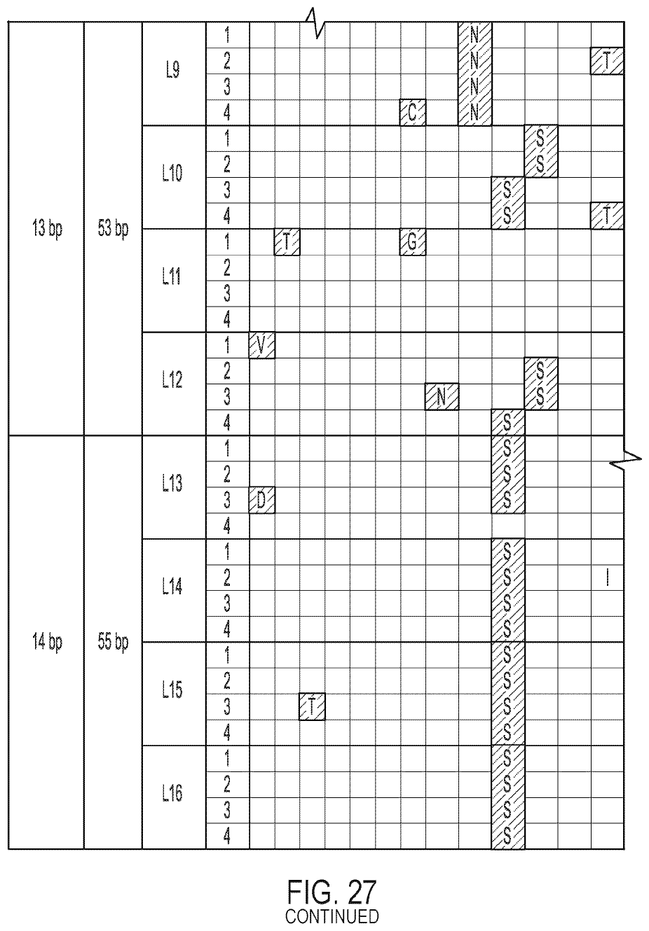

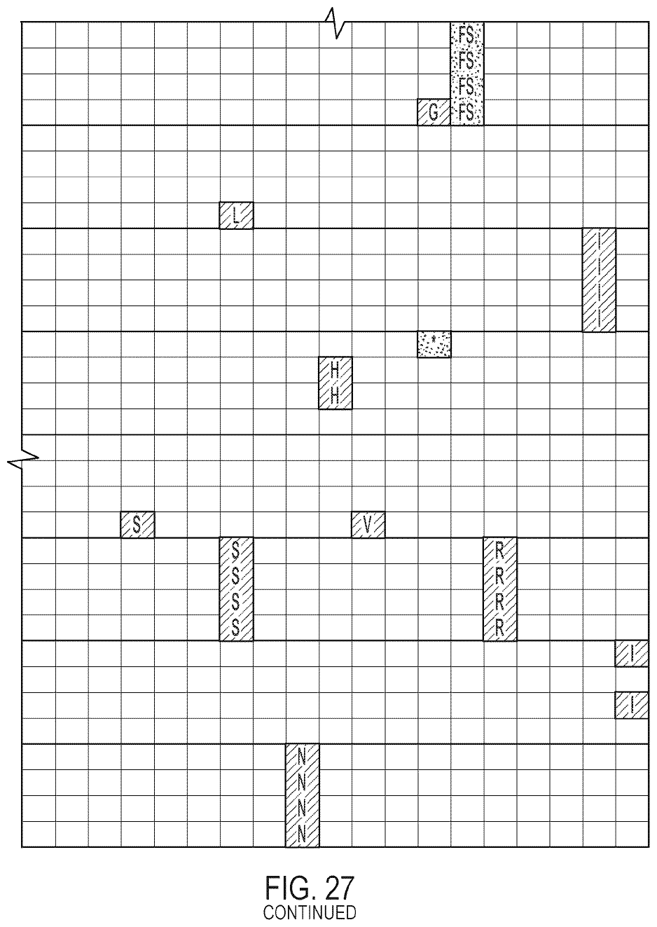

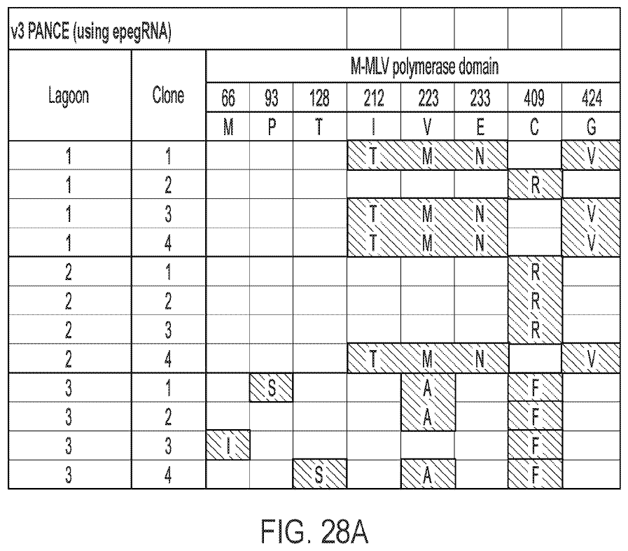

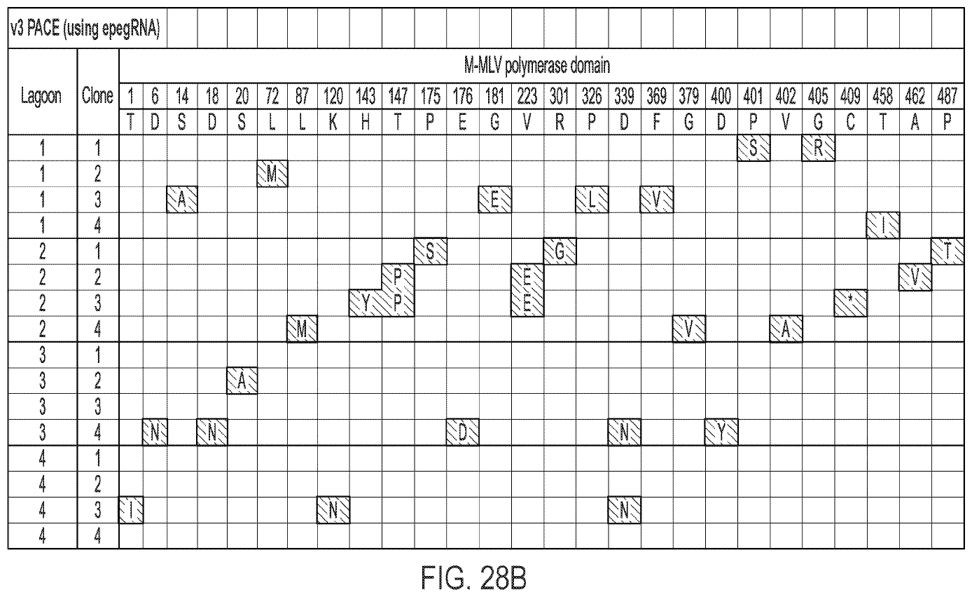

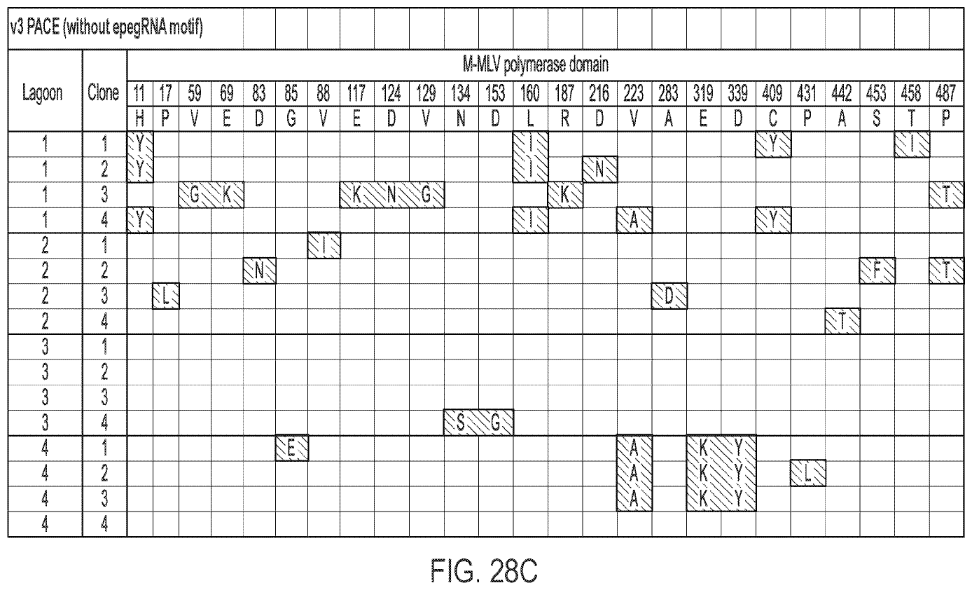

A different approach was used to evolve and engineer the M-MLV RT. The high starting activity of PE2's already-engineered RT enabled it to be evolved in parallel, rather than successive, experiments using the v1-v3 circuits to maximize the likelihood of discovering RT mutations that benefit PE efficiency for different types of edits (FIG. 4A). In addition to the five engineered mutations already present in PE2 compared to wild-type M-MLV RT, over 20 new mutations showing varying levels of convergence emerged from v1 PACE, v2 PANCE, and v3 PANCE (FIGS. 26, 27, 28A-28C). One cluster of mutations emerging from high-stringency v2 and v3 PANCE was particularly promising (FIG. 4B): T128N, V129A/G, P196S/T/F, N200S/Y, and V223A/M/L/E. All five of these residues are predicted to be near the polymerase active site, and the N200 and V223 positions are known to be particularly important to prime editing activity. Indeed, N200 itself was the most impactful single mutation that was previously installed in M-MLV RT to create PE2¹. Similarly, V223 is part of the core YXDD motif that has been implicated in the activities of numerous different RTs⁴⁶.

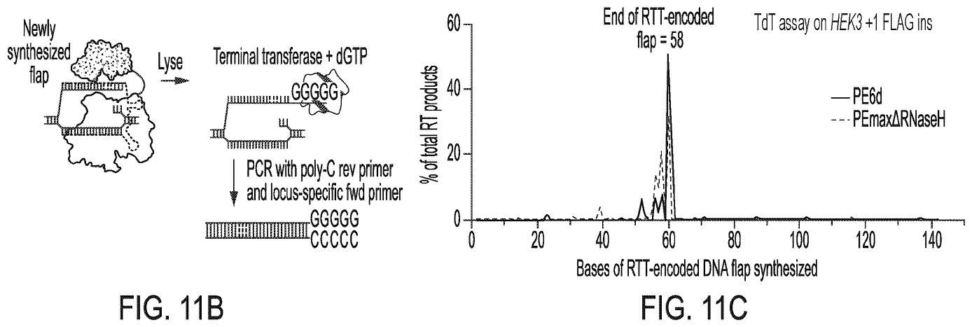

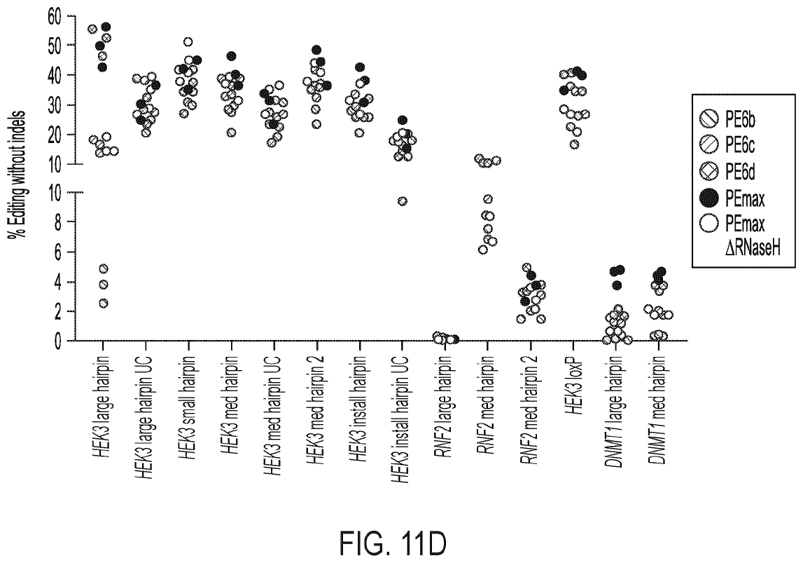

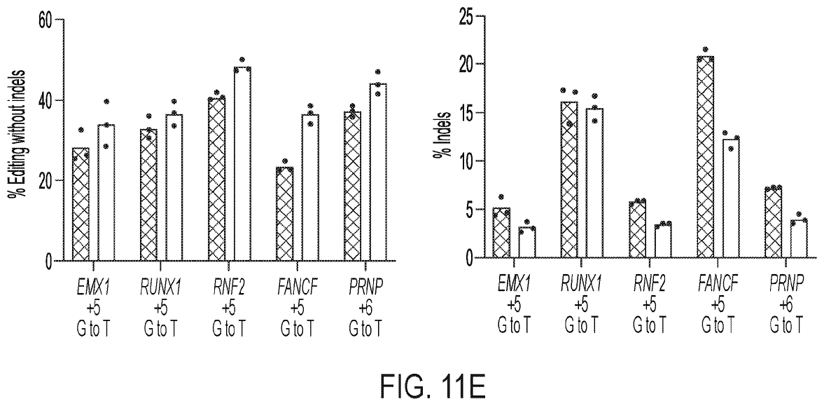

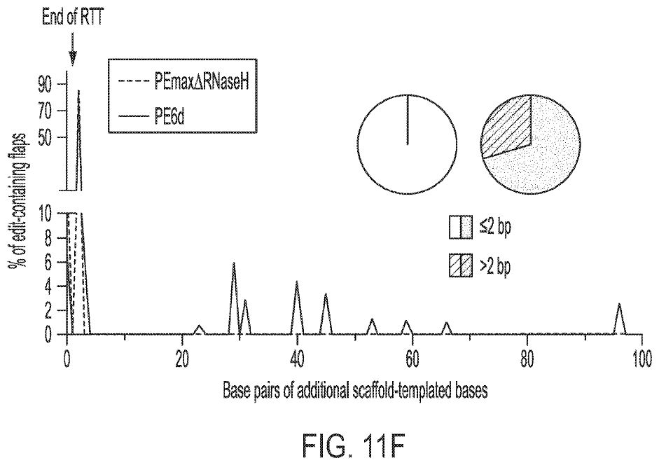

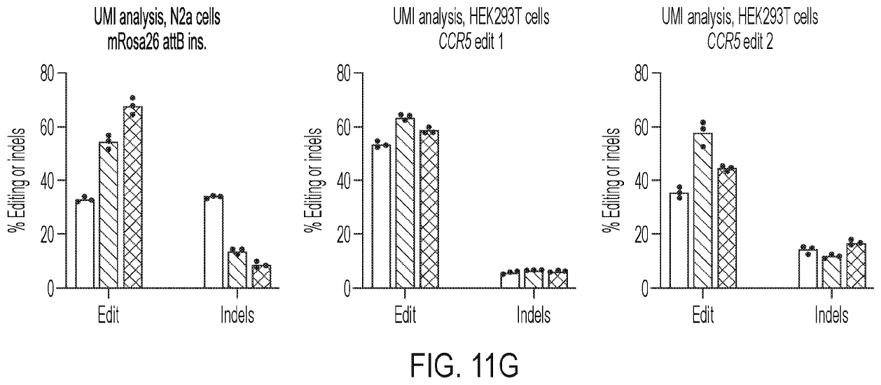

Because the goal of this evolution was to create a highly active size-minimized M-MLV variant, evolution of PEmaxΔRNaseH was also originally planned. Interestingly, explicit deletion of the RNase H domain was not necessary; in addition to conserved single amino acid changes, evolved M-MLV RT variants also harbored mutations such as Q492* (stop) or H503FS (frameshift), which truncate the RT between its polymerase domain and its RNaseH domain (FIG. 4B), remarkably close to where M-MLV has been truncated via protein engineering^{23,29,30}. Thus, high-stringency M-MLV RT evolution enriched mutations at residues known to be important for prime editing efficiency and RT activity, and some variants evolved RNaseH domain truncations that facilitate in vivo prime editor delivery. Evolved mutations and engineered mutations were screened at the same conserved residues, and then the most promising candidates were combined to generate an RNaseH-truncated evolved and engineered M-MLV variant that was named PE6d (three of the PE2 mutations (T306K, W313F, and T330P)+T128N+D200C+V223Y, FIG. 11A).

### Dependence of PE6c, PE6d, and PEmaxΔRNaseH Performance on RTT Secondary Structure

PE6c and PE6d were compared to dual AAV-compatible PEmaxΔRNaseH, as well as full-length PEmax, at several longer prime edits and twinPE edits in HEK293T cells. Both PE6c and PE6d were able to recover prime editing efficiency for long edits relative to PEmaxΔRNaseH, with PE6c or PE6d matching or surpassing PEmax's editing efficiency at all four edits tested (FIG. 4C). It was noted that PEmaxΔRNaseH did not always exhibit deficiencies at long edits compared to PEmax, PE6c, and PE6d. To investigate what dictates the relative efficiencies of these prime editors, and to enable a priori prediction of which prime editor is best to use for a given edit, the enzymatic determinants of PE efficiency was investigated more deeply.

Until this point, edits were classified based on their RTT length, but this feature alone did not fully account for the differences that were observed between enzymes. For instance, both the HEK3+1 FLAG insertion and the HEK3+1 loxP insertion pegRNAs require the use of a long RTT (58 bp and 74 bp respectively) and have identical spacer and PBS sequences, but the efficiency of PEmaxΔRNaseH and PE6d differed substantially between the two edits. While the two prime editors performed comparably at the FLAG insertion, PE6d offered 1.9-fold higher editing efficiency than PEmaxΔRNaseH for the loxP insertion (FIG. 4D). To probe this discrepancy, the predicted secondary structure of the two pegRNAs' 3′ extensions were examined using NUPACK⁴⁷, and it was found that the FLAG insertion pegRNA 3′ extension is largely disordered, whereas the loxP insertion 3′ extension contains a strong 13-bp hairpin (FIG. 4D). This observation led to the hypothesis that RTT secondary structure dictates the relative efficiencies of PEmaxΔRNaseH and certain highly evolved PEs such as PE6c and PE6d.

To more directly measure the effects that a structured RTT might have on the performance of different RT variants, a terminal deoxynucleotidyl transferase (TdT) assay was conducted^{1,5} using PEmaxΔRNaseH and PE6d at the HEK3 FLAG insertion and loxP insertion edits. In the TdT assay, cells are transfected with prime editing machinery and then lysed after 24 hours to capture and sequence the newly reverse-transcribed DNA flap that has not yet been incorporated into the genome (FIG. 11B). Interestingly, when HEK3+1 loxP insertion DNA flaps were sequenced, it was found that 30% of PEmaxΔRNaseH products prematurely truncated at bases that were templated by the beginning of the hairpin in the pegRNA RTT. This prematurely truncated RT product was much less common in PE6d-synthesized DNA flaps (only 5.8% of RT products). As a result of fewer premature truncation events, PE6d was more often able to produce full-length DNA flaps that contained that entire RTT-encoded sequence (62% of RT products as opposed to 34% of PEmaxΔRNaseH RT products, FIG. 4E). In contrast, at the HEK3 FLAG insertion edit for which the two editors perform similarly, both enzymes were able to produce the full-length RT product most of the time (78% of RT products for PE6d and 70% of RT products for PEmaxΔRNaseH, FIG. 11C). These data suggested a mechanism of how RTT secondary structure might dictate relative editing efficiency: RNaseH domain truncation, which has been shown in vitro to decrease enzyme processivity⁴⁸, causes prematurely terminated RT products that lack the edit or the downstream homology region and are thus unproductive for prime editing. However, the polymerase domain mutations in PE6d can compensate for the lack of the RNaseH domain, restore enzyme processivity, and synthesize full-length products despite secondary structure in the pegRNA RTT.

To systematically interrogate whether other sites and edits support the emerging hypothesis that RTT secondary structure determines which prime editor performs best for a long edit, a series of different pegRNAs were engineered that contained long, stable hairpins. “Unpinned” controls in which two to four point mutations minimally changed the pegRNA sequence but strongly disrupted the pegRNA secondary structure were also included. PEmaxΔRNaseH and PE6d were compared using this set of pegRNAs. It was found that PE6d strongly outperformed PEmaxΔRNaseH when RTTs contained strong hairpins, yielding a 2.3-fold average improvement in editing efficiency (FIG. 4F, FIG. 11D). In contrast, for the corresponding unpinned control RTTs with disrupted hairpins, the two prime editors performed nearly identically. These results strongly supported the idea that secondary structure, rather than RTT length alone, determines the relative efficiencies of PE6d and PEmaxΔRNaseH.

Next, it was investigated whether computational predictions of pegRNA folding energetics could accurately identify which dual AAV-compatible PE is best for a given edit. The hairpin edits described above were aggregated, along with additional edits, and the relationship between the NUPACK-predicted free energy of RTT/PBS folding and the difference in editing efficiency between PE6d and PEmaxΔRNaseH was compared. It was found that when the predicted free energy of folding of the RTT and PBS was less favorable than −23 kcal/mol, PE6d was equally efficient or less efficient than PEmaxΔRNaseH. However, when the predicted free energy of folding was more stable than −23 kcal/mol, PE6d offered large improvements, ranging from 1.3-fold to 3.1-fold relative to PEmaxΔRNaseH. This relationship is a simple and useful tool for selecting a prime editor variant: predicting the free energy of RTT/PBS folding can greatly inform the choice of prime editor variant. (This measure of free energy of folding does not take the pegRNA spacer, scaffold, or epegRNA 3′ pseudoknot motif into account, as they are not directly engaged by the RT.)

It was also noted that when pegRNA RTTs were short and unstructured, PE6d tended to produce lower editing efficiencies and higher indel frequencies than PEmaxΔRNaseH (FIG. 11E). Upon examining the nature of the indels produced and performing the TdT assay on a representative edit (RNF2+5 G to T), it was discovered that PE6d catalyzed an increased rate of scaffold insertion relative to PEmaxΔRNaseH when a short, unstructured RTT was used (FIG. 11F). Scaffold insertion is a known byproduct of prime editing in which reverse transcription of the sgRNA scaffold produces undesired bases at the end of the genomic DNA flap. While reverse transcription products containing scaffold nucleotides are likely cleaved off as 3′ flap termini with no DNA base pairing to the unedited DNA strand, these additional bases can impede flap equilibration or lead to scaffold incorporation into the target site, especially if some scaffold nucleotides share adventitious homology with the target site. Mechanistically, it makes sense that PE variants that are able to overcome RTT secondary structure would also increase this type of undesired byproduct, leading to reduced precise editing for short-RTT edits. As a result, PE6d is not well suited for small prime edits. Interestingly, general increases in indels (FIGS. 4H-4J) or scaffold insertion (FIG. 4E and FIG. 11C) were not observed when PE6d was used with a long, structured RTT. It was thought that the RTT itself acts as a buffer to prevent the RT from reading into the sgRNA scaffold, and that modest levels of reverse transcription into the scaffold create non-homologous 3′ ends that are typically removed by cellular nucleases, resulting in minimal scaffold incorporation. It is noted that PE6d and other processive RTs do not uniformly increase indels at the edit types for which they are most useful; substantial increases in scaffold incorporation were only observed when an RT is more processive than is required for a specific edit (for example, using PE6d with a short, unstructured RTT).

This discovery also offers important insights into prime editing. For a given edit, there is an optimal level of reverse transcriptase activity that balances successful generation of RTT-templated bases, with avoidance of reverse transcription into the sgRNA scaffold. This result also agrees with early PACE results and explains why RTs evolved in the v2 selection, which used a long, hairpin-containing RTT, became less fit in the v1 selection, which uses a short RTT.

Similar processivity analyses were performed on Tf1 variants PE6b (which is less processive) and PE6c (which is more processive) and found a similar relationship between these two enzymes (FIG. 11D). While generally not as active as PE6d, it was found that PE6c outperformed PEmaxΔRNaseH at most highly structured edits (FIG. 11D). PE6b appeared to have a level of processivity similar to PEmaxΔRNaseH, which makes it a promising candidate for the installation of edits that require a short, unstructured RTT.

It was expected that in addition to highly structured prime edits, PE6c and PE6d would improve most twinPE efficiencies, which typically use long RTTs. Therefore their activity was evaluated compared to PEmaxΔRNaseH at a variety of twinPE edits in HEK293T cells. It was found that PE6 variants offered improvements in efficiency relative to PEmaxΔRNaseH, with the PE6c yielding a 1.6-fold average improvement across the five sites tested (FIG. 4H). To minimize potential PCR bias that can arise during sample preparation for large twinPE edits¹⁰, unique molecular identifiers (UMI) were applied to quantify a subset of twinPE edits (FIG. 11G) to confirm that PE6c and PE6d variants can offer substantial advantages over PEmaxΔRNaseH. Importantly, it was noted that PE6c and PE6d did not substantially alter the editing:indel ratio for these twinPE edits.

Finally, the ability of PE6 variants to perform longer prime edits was also examined in two mouse genomic targets in N2a cells. A twinPE strategy was optimized for insertion of the BxbI recombinase attB recognition sequence at the murine Rosa26 safe harbor locus, and it was found that PEmaxΔRNaseH generated on average 31% installation of the edit but also yielded an equal number of indels. Conversely, PE6c and PE6d both increased editing efficiency and decreased indel rates at this site, with PE6d yielding an 8.6-fold increase in the editing:indel ratio for this edit (FIG. 4I). Similarly, a strategy for the installation of a loxP sequence at the murine Dnmt1 locus was optimized. Relative to PEmaxΔRNaseH, PE6d enhanced editing efficiency by 2.1-fold and increased the editing:indel ratio by 1.7-fold (FIG. 4J). The twinPE and N2a data shown here suggests that, unlike at short-RTT edits, highly processive RTs do not substantially increase levels for long, structured RTTs. It is possible that long RTTs act as a buffer to prevent scaffold insertion. Overall, these results indicate that for dual-AAV compatible editors, PE6c and PE6d offer substantial improvements over PEmaxΔRNaseH for many different types of challenging edits.

PE6 Variants with Different Processivity Offer Improvements Over PEmax

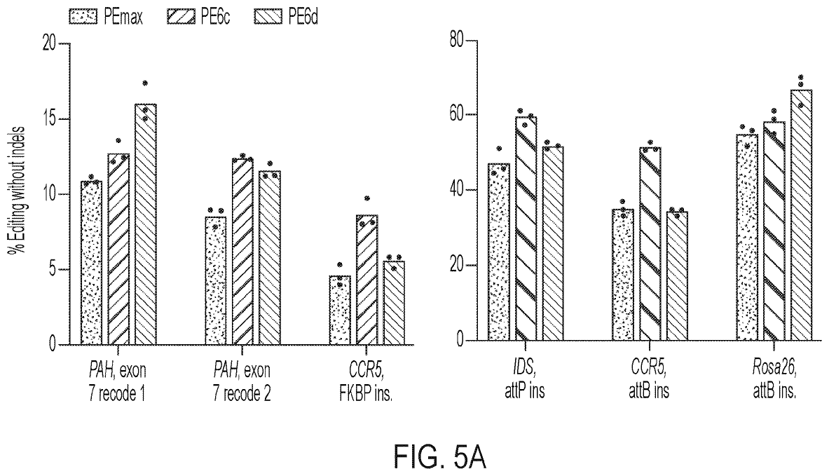

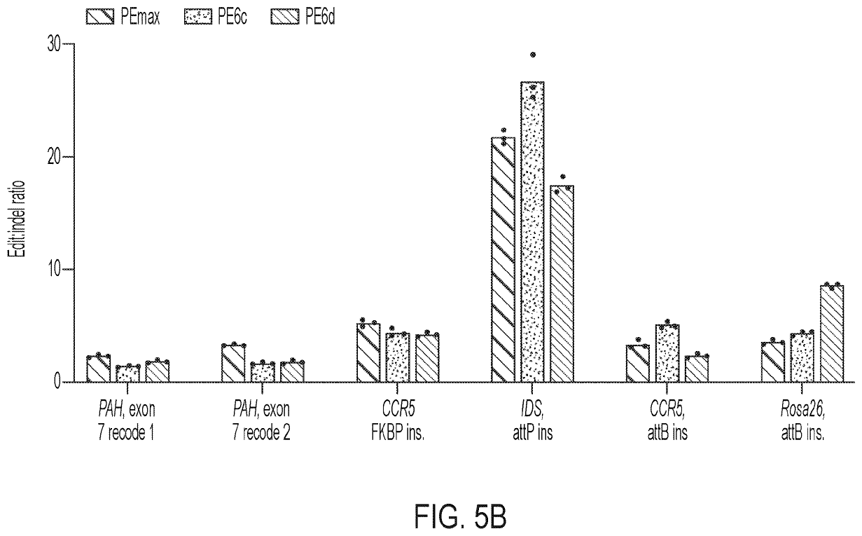

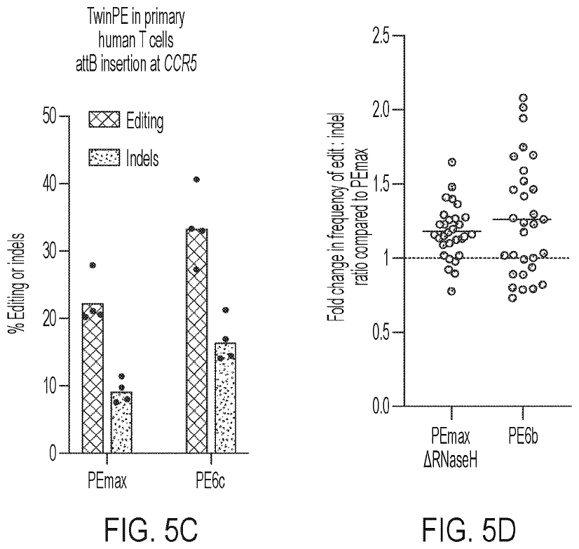

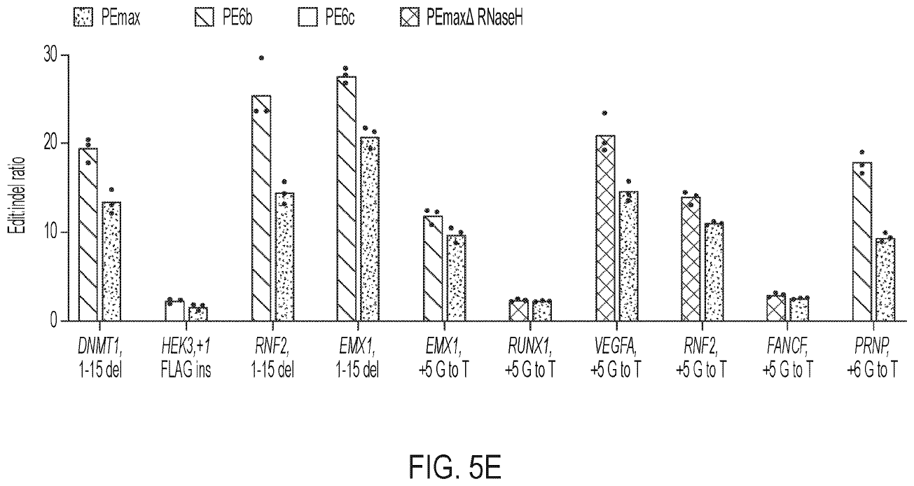

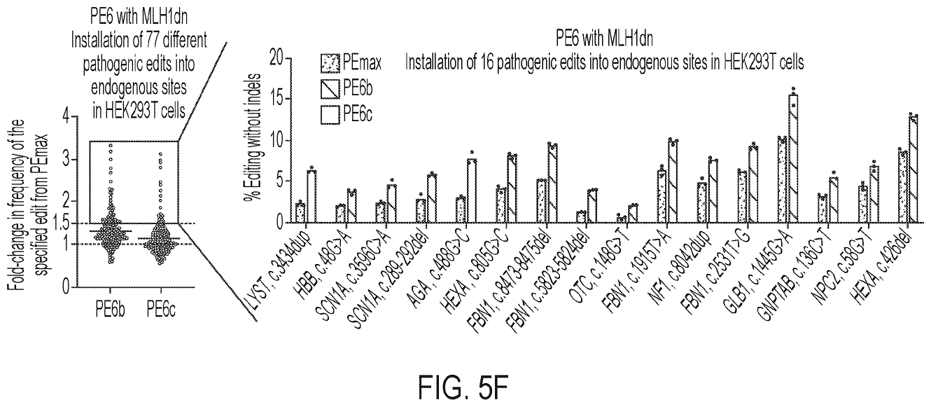

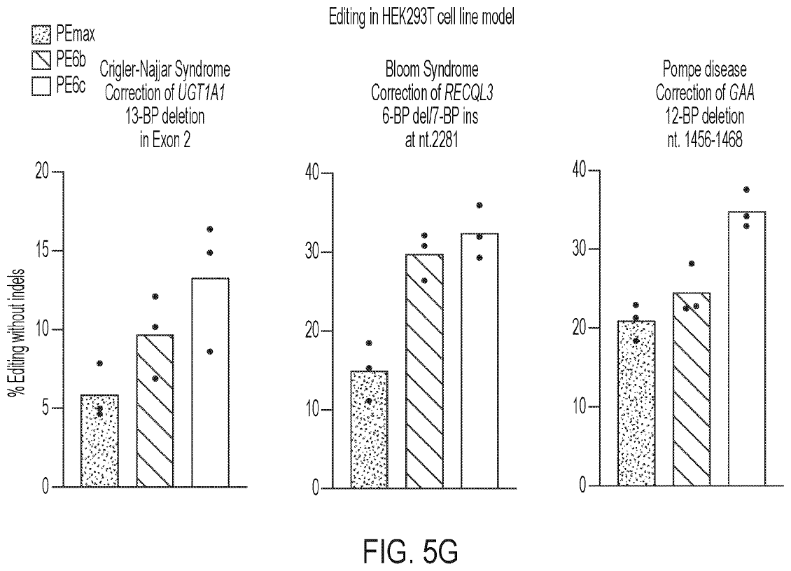

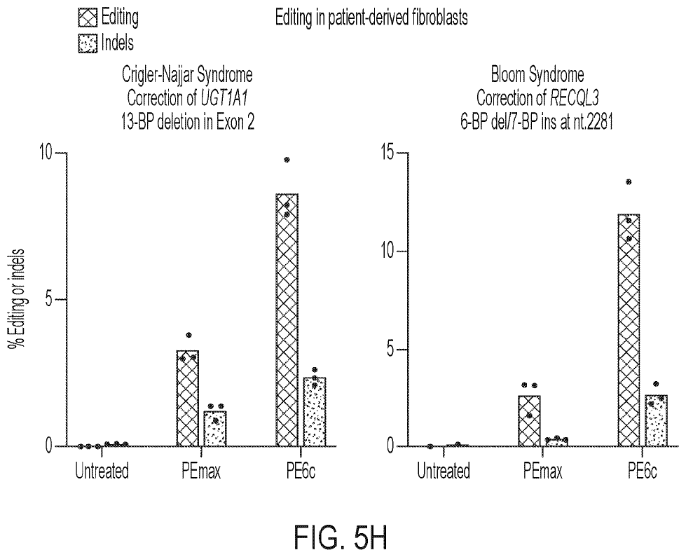

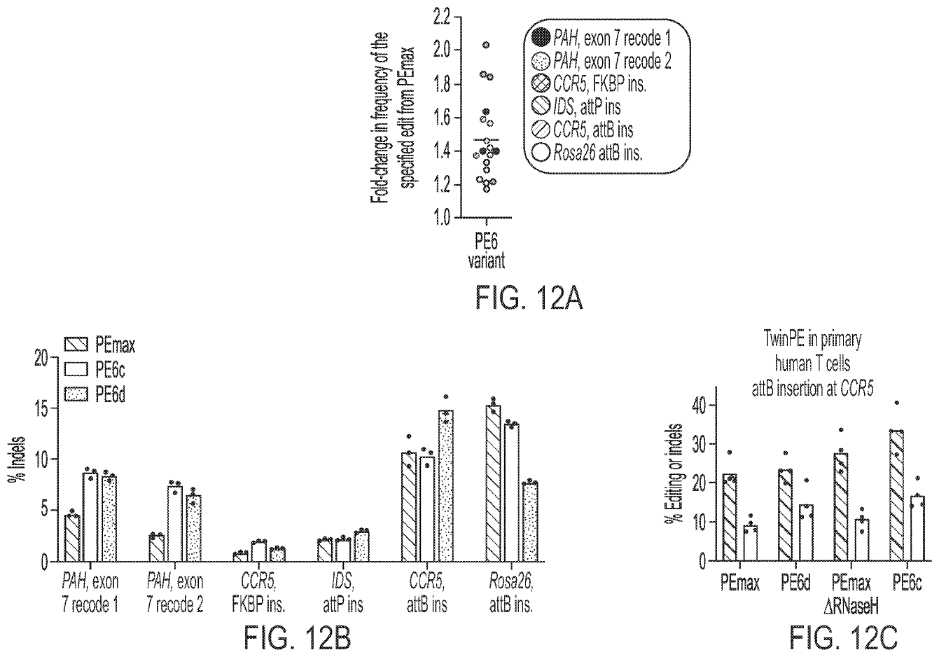

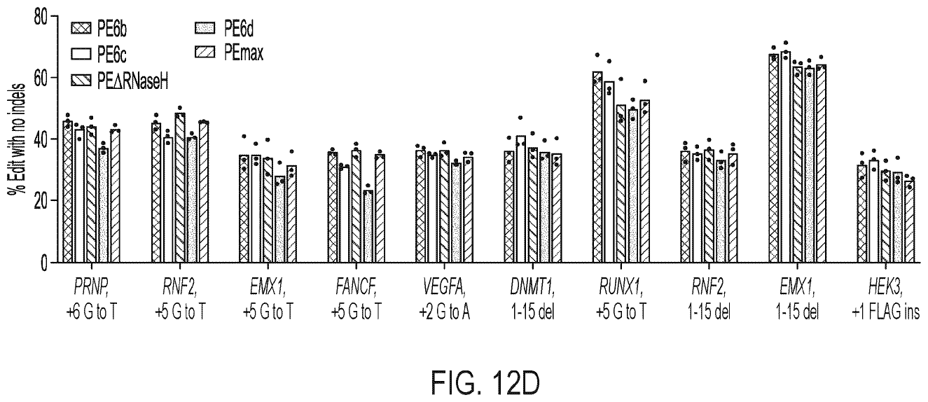

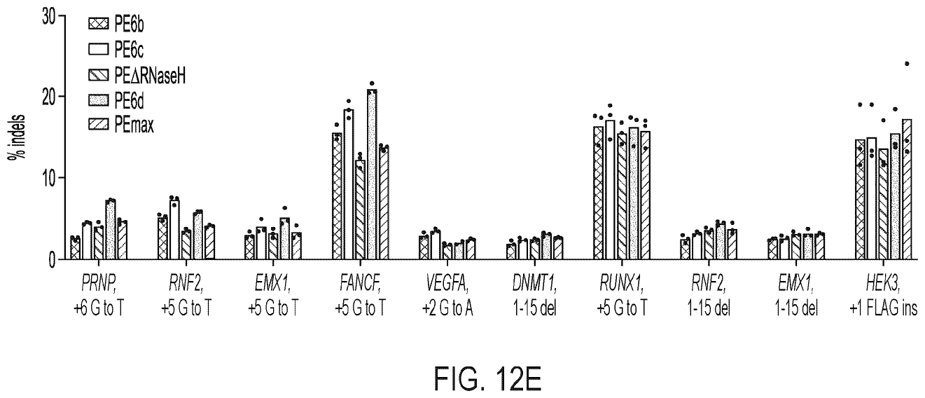

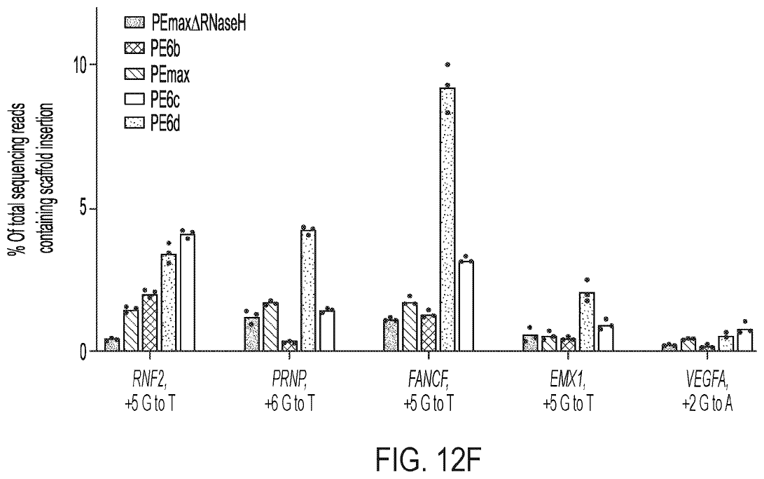

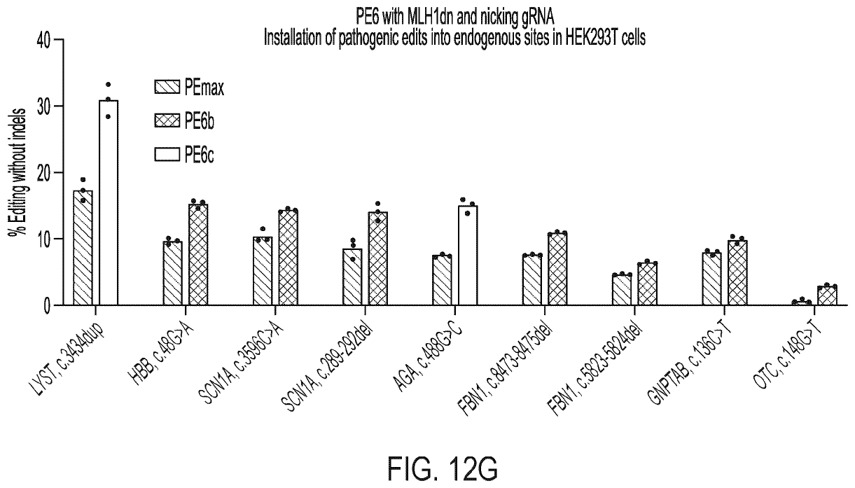

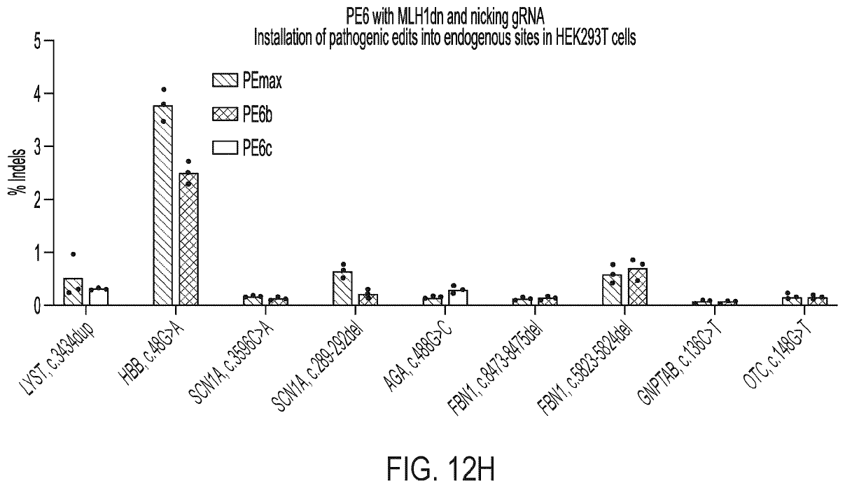

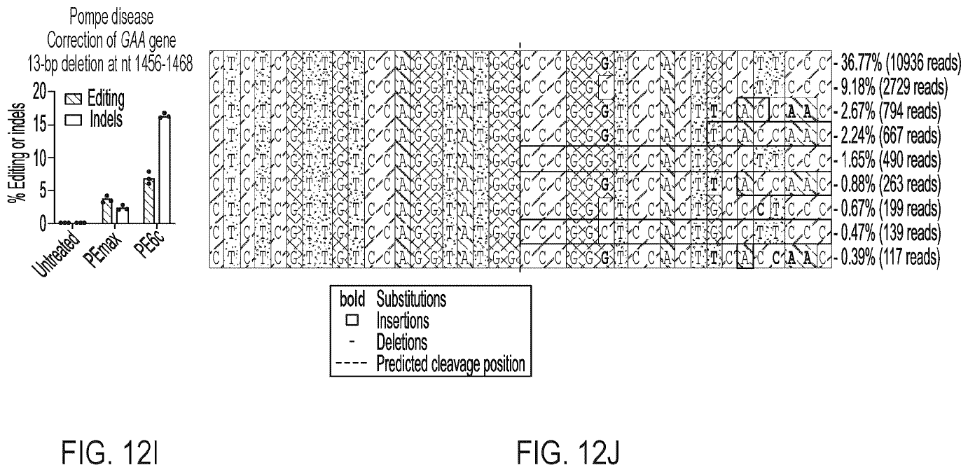

Next, whether PE6a-d variants could offer improvements over PEmax was investigated. Given PE6c and PE6d's enhanced processivity, they may offer improvements over PEmax for longer prime edits. Therefore PEmax, PE6c, and PE6d were tested at six twinPE edits that included (i) recoding 43 bp of exon 7 in the PAH gene, (ii) inserting a 108-bp fragment of FKBP12's cDNA into the CCR5 locus in HEK293T cells, (iii) inserting a 38 bp recombinase attachment site (attB) into the murine safe harbor Rosa26 locus and human safe harbor CCR5, and (iv) inserting a 50-bp recombinase attachment site (attP) in the IDS gene. PE6 variants improved editing efficiency by 1.4-fold over PEmax across all of these edits (FIG. 5A, FIG. 12A) without substantially changing the edit:indel ratio (FIG. 5B, FIG. 12B).

PEmax and PE6 variants were also tested for attB insertion at the CCR5 safe harbor locus in primary human T cells. PE6c offered a 1.5-fold improvement in editing efficiency relative to PEmax, achieving an average attB insertion efficiency of 34% across T cells from four different donors (FIG. 5C, FIG. 12C). These results represent the first report of twinPE in primary human T cells and confirm that PE6 variants offer substantial improvements for therapeutically relevant prime editing.

The previous data characterizing PE6c and PE6d demonstrated that high RT processivity can be detrimental for the installation of edits that use short, unstructured RTTs. The same phenomenon may therefore apply to PEmax. Since PE6b and PEmaxΔRNaseH have reduced RT processivity compared to PEmax (as approximated by their lower performance for long edits), it was reasoned that they could lead to improvements in editing:indel ratios achieved for small, unstructured edits as a result of reduced pegRNA scaffold incorporation. Therefore, PE6b and PEmaxΔRNaseH were tested alongside PEmax at ten unstructured RTT edits that encoded SNPs, insertions, or deletions and had a NUPACK-predicted free energy between 0 to −12 kcal/mol. Indeed, it was observed that both PE6b and PEmaxΔRNaseH had more favorable edit:indel profiles than PEmax (FIG. 5D, FIGS. 12D-12E). For every edit tested, PEmaxΔRNaseH or a PE6 variant offered a higher editing:indel ratio than PEmax (FIG. 5E). Upon examining the indels generated by these variants for a subset of edits, it was observed that PE6b and PEmaxΔRNaseH indeed incorporated the pegRNA scaffold bases less frequently than PEmax (FIG. 12F). Collectively, these data support that PE6b and PEmaxΔRNaseH are more favorable for edits with unstructured RTTs due to their lower processivity, which results in less incorporation of scaffold bases into the genome and ultimately improves edit:indel ratios.

### Alternate RT Domain Tf1 Offers Improvements Over the PEmax RT Domain for Several Therapeutic Edits

Finally, it was investigated whether alternate RT domains could improve editing efficiencies compared to PEmax for reasons other than relative enzyme processivity. For other genome editing tools such as Cas9 nucleases and base editors, a diversity of orthologues has greatly increased the likelihood of finding an enzyme that is ideally suited for a particular application²². Unfortunately, M-MLV has been the only RT previously reported to support efficient prime editing. Without other efficient RT domains to compare, it is unclear if editing trends resulted from prime editing in general or from the preferences of the M-MLV RT. To further characterize this phenomenon, PE6b and PE6c, both of which were derived from the Tf1 RT, were tested at many additional loci alongside PEmax.

First, 77 pegRNAs⁴⁰ were tested that install disease-associated edits into endogenous sites in HEK293T cells, and these were transfected along with MLH1dn (but no nicking sgRNA) and each PE variant. It was observed that on average, both PE6b and PE6c slightly outperformed PEmax (FIG. 5F). PE6b demonstrated a 1.3-fold improvement in average editing efficiency over PEmax, while PE6c showed a 1.1-fold average improvement. Out of the 77 sites tested, PE6b and/or PE6c substantially outperformed PEmax (1.5-fold or more) at 16 sites. In some cases, improvements in editing efficiency were even larger (up to 3.1-fold higher than PEmax) (FIG. 5F). Several sites in which PE6b and/or PE6c improved editing efficiencies were chosen, and nicking guide RNAs that target the non-edited strand were added to enhance editing efficiency (the “PE7” strategy). For all edits, PE7b or PE7c continued to substantially outperform PEmax without increasing indel levels (FIGS. 12G and 12H) and for most edits, addition of a nicking gRNA further improved editing efficiencies. It is envisioned that PE6 and PE7 variants along with the set of epegRNAs used in this experiment will be useful to generate a wide range of cell models of genetic disease using prime editing.

Next, to examine the potential utility of Tf1-derived editors for disease correction, Sleeping Beauty transposase⁴⁹ was used to integrate pathogenic alleles into the genomes of HEK293T cells. Using this technique, model cell lines were created that harbored pathogenic mutations known to cause Glycogen Storage Disease II (Pompe Disease), Bloom Syndrome, and Crigler-Najjar Syndrome. PEmax, PE6b, and PE6c were then evaluated for their ability to correct each pathogenic mutation. For all three edits, PE6c generated the highest average editing efficiency (13-35%), a 2.1-fold average increase over PEmax across the three model cell lines (FIG. 5G). PEmax and PE6c were next tested in patient-derived fibroblasts that harbored the same pathogenic alleles. PE6c-mediated improvements in editing efficiencies were more pronounced in patient-derived fibroblasts. PE6c improved average editing efficiency by 4.5-fold in fibroblasts derived from a Bloom Syndrome patient, and 2.6-fold in cells derived from a Crigler-Najjar Syndrome patient. In the Pompe disease patient-derived fibroblasts, PE6c improved indel-free editing efficiencies relative to PEmax by 1.9-fold (FIGS. 12I, 12J). Interestingly, many of the indels detected at this site did not contain the silent PAM edit encoded by the pegRNA, suggesting those indels were not RT-templated products. Collectively, these data show that the RT variants generated herein can repeatedly outperform the highly engineered M-MLV RT currently used in PEmax in a variety of disease-relevant contexts and cell types.

### Evolution of Cas9 Variants for Enhanced Prime Editing

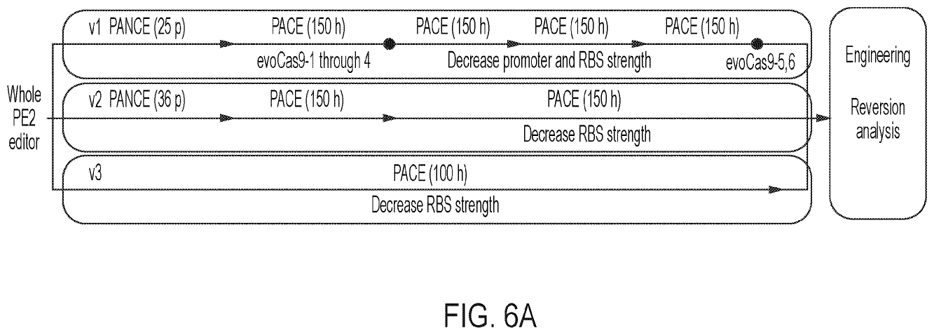

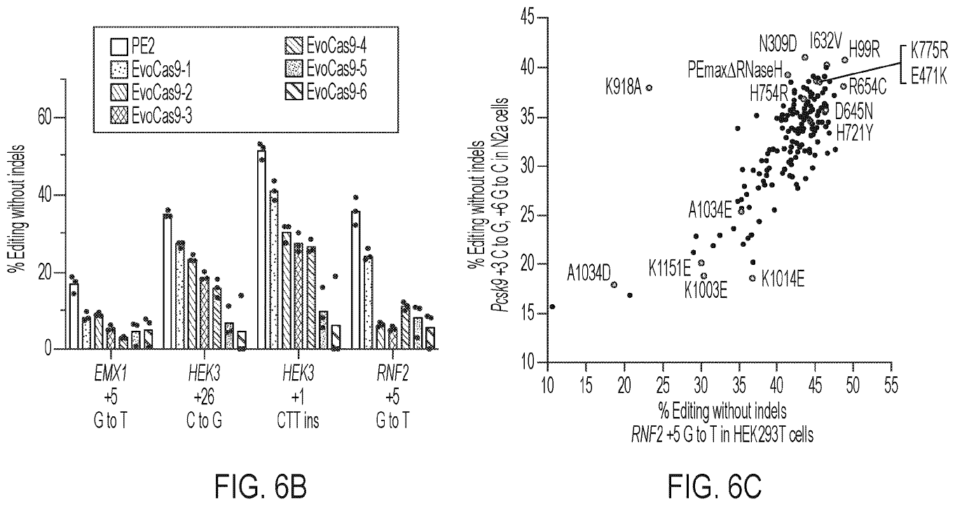

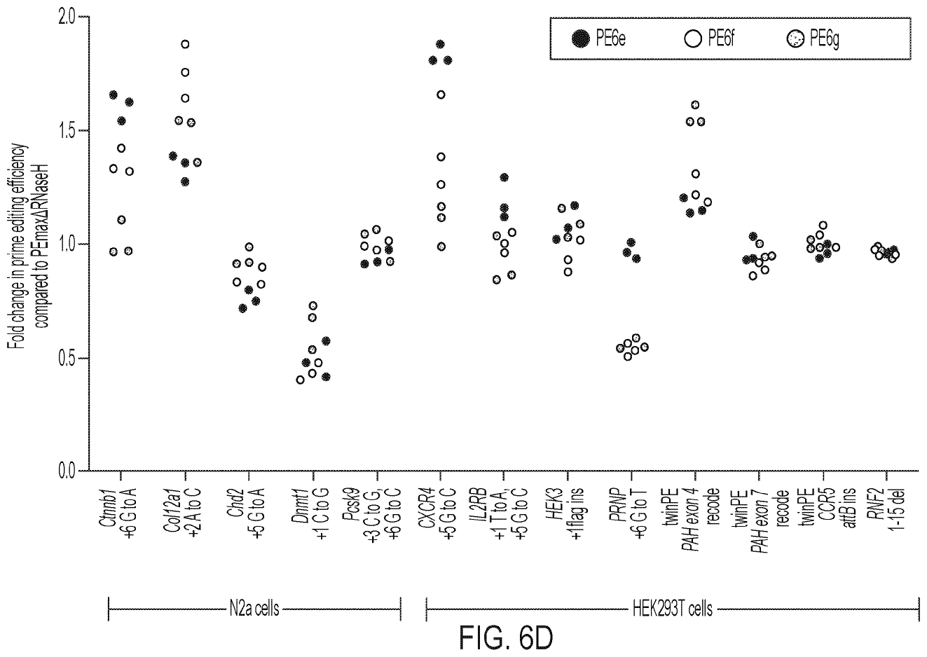

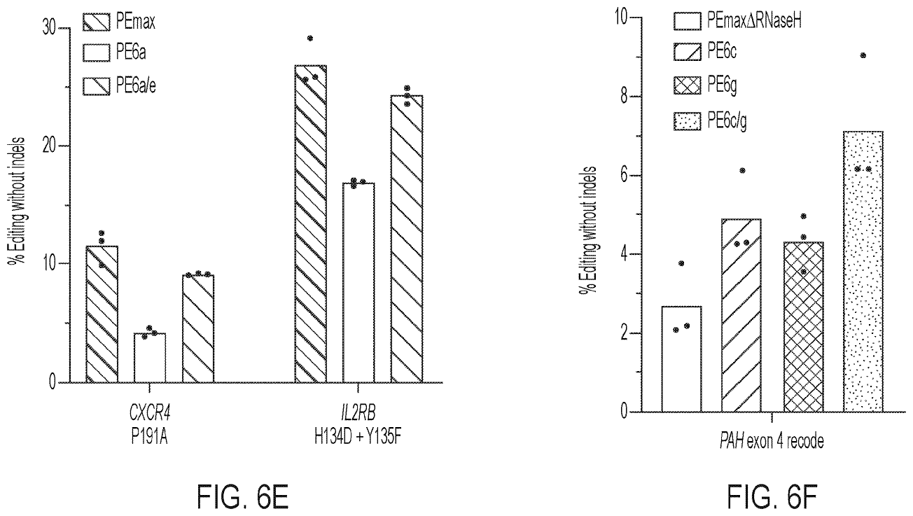

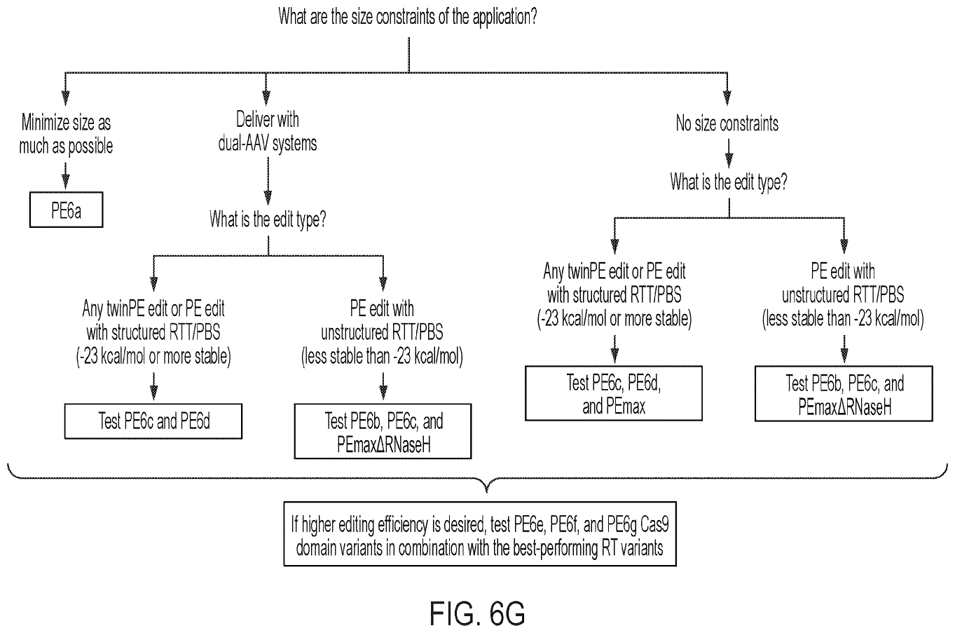

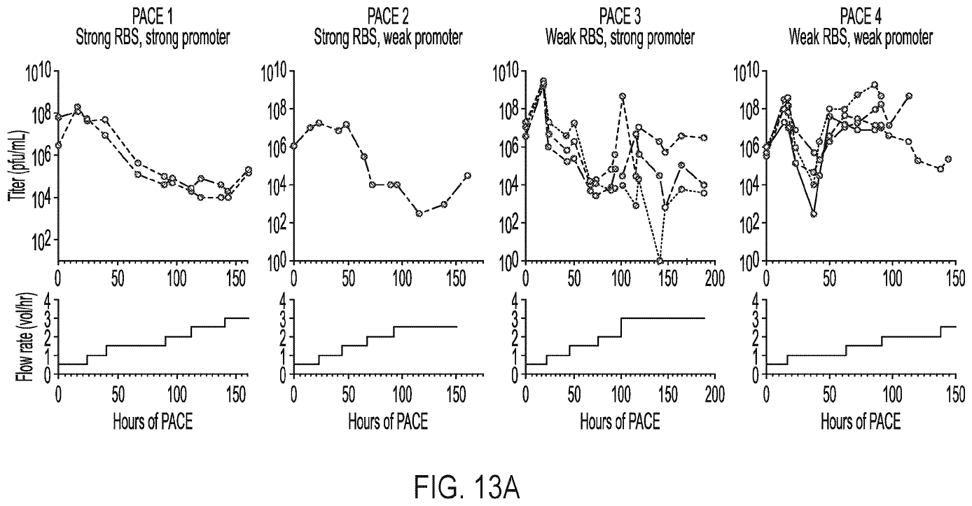

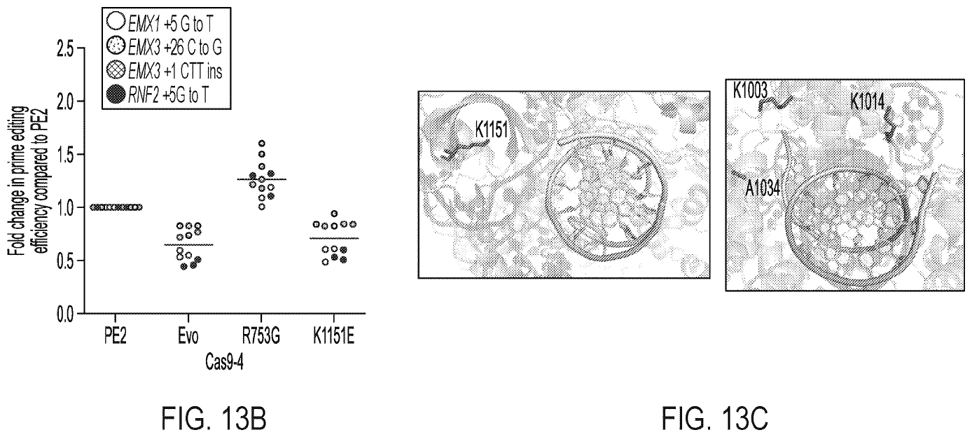

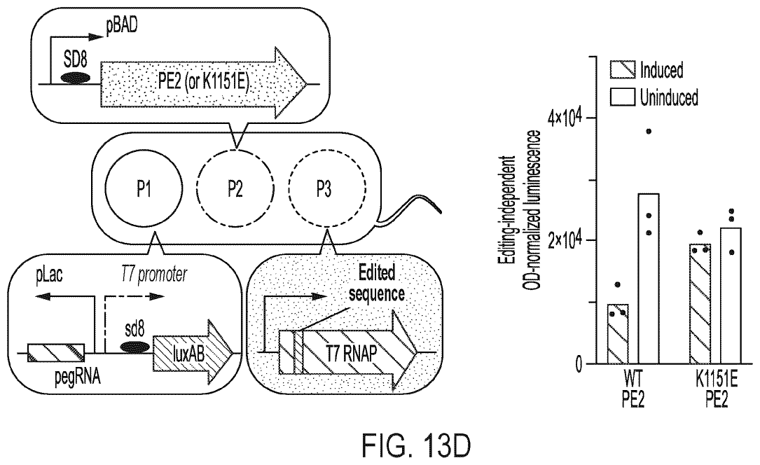

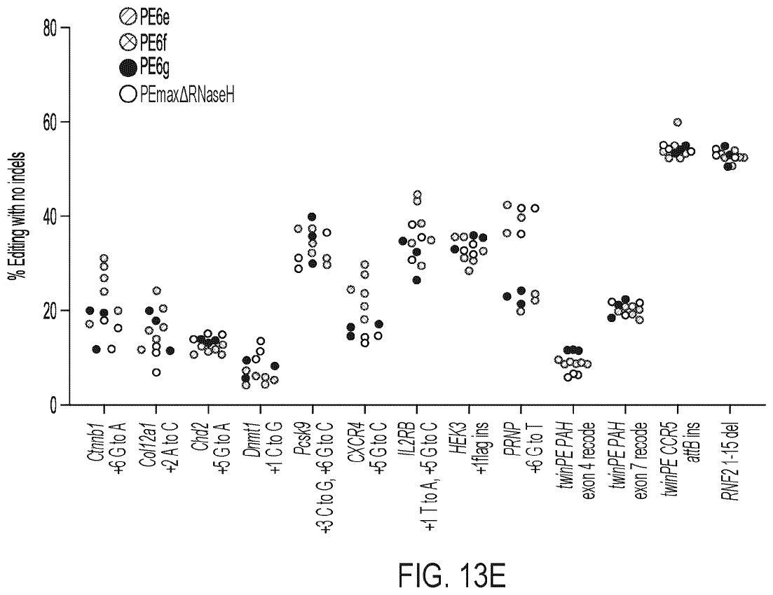

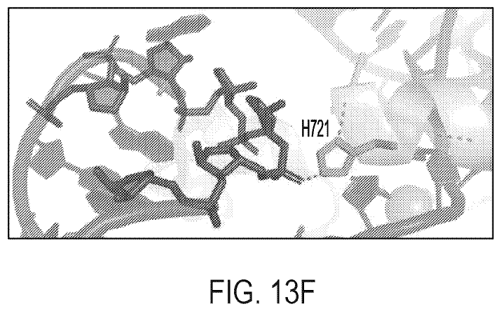

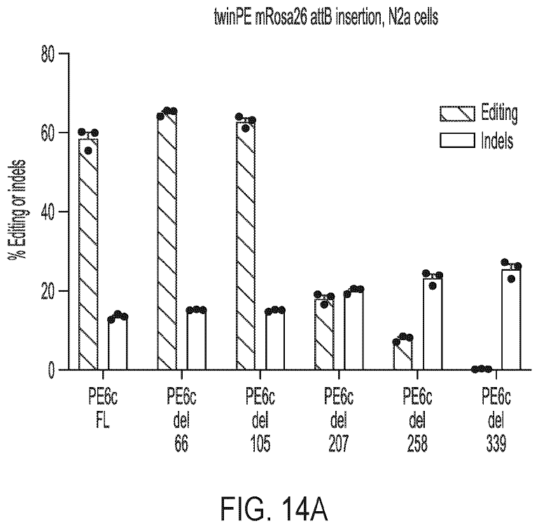

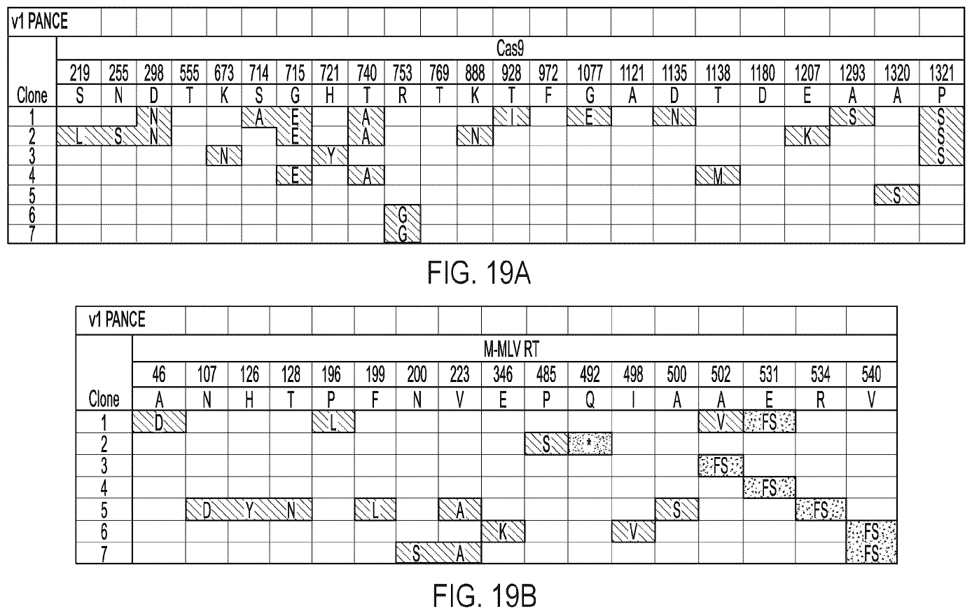

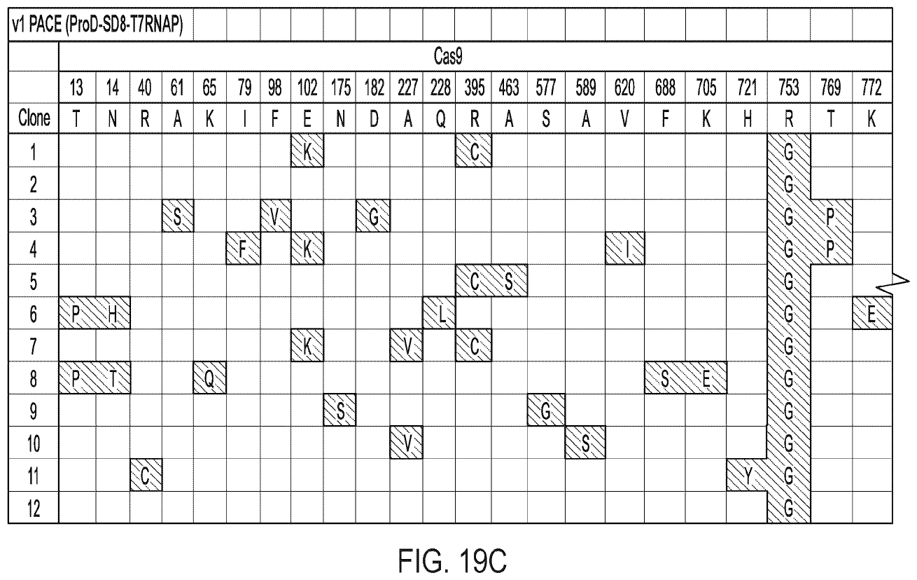

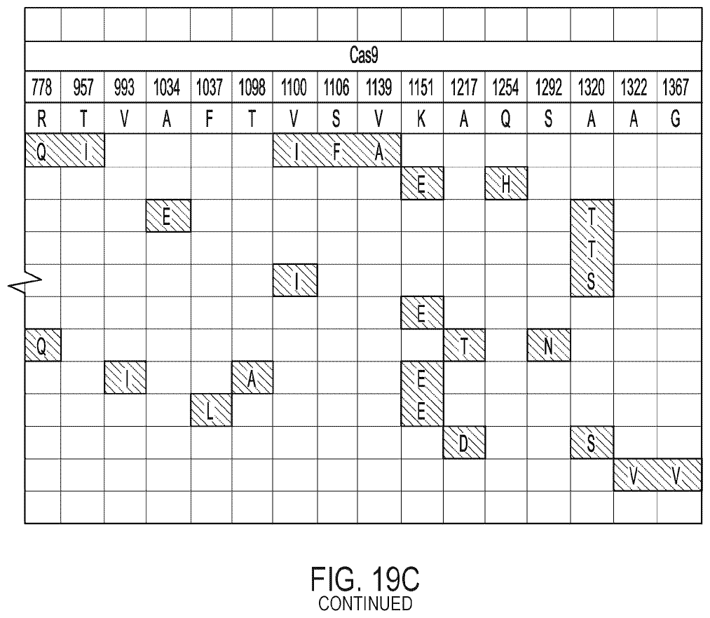

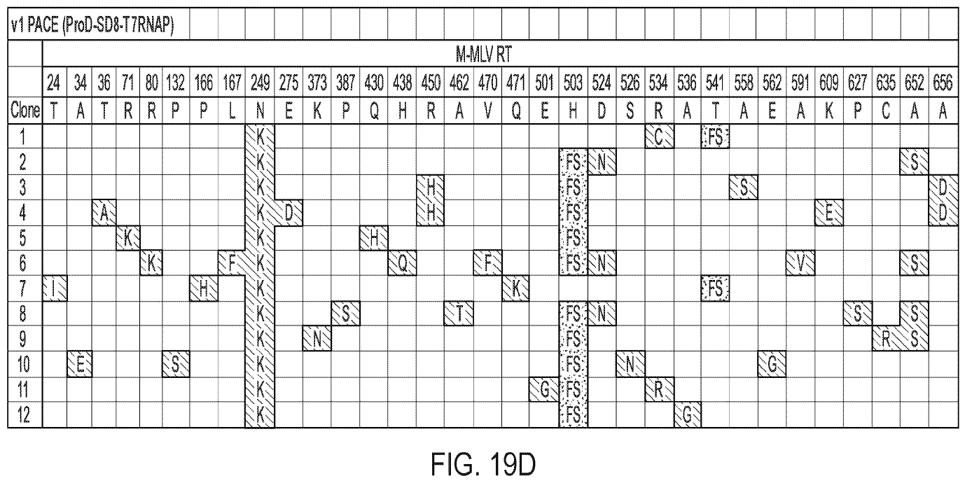

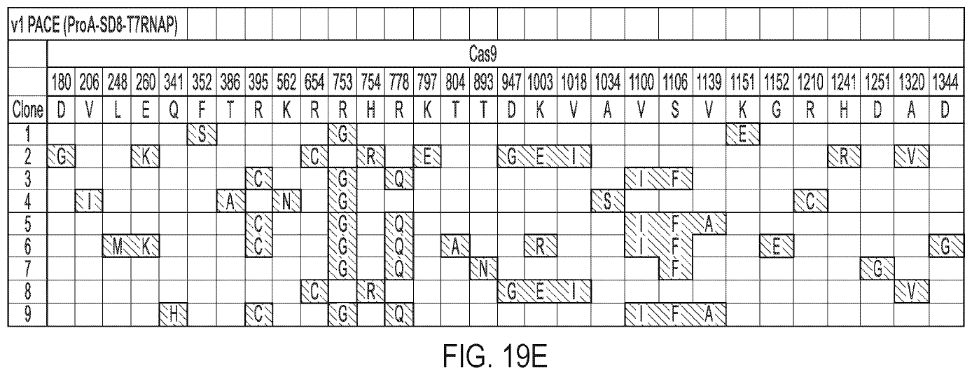

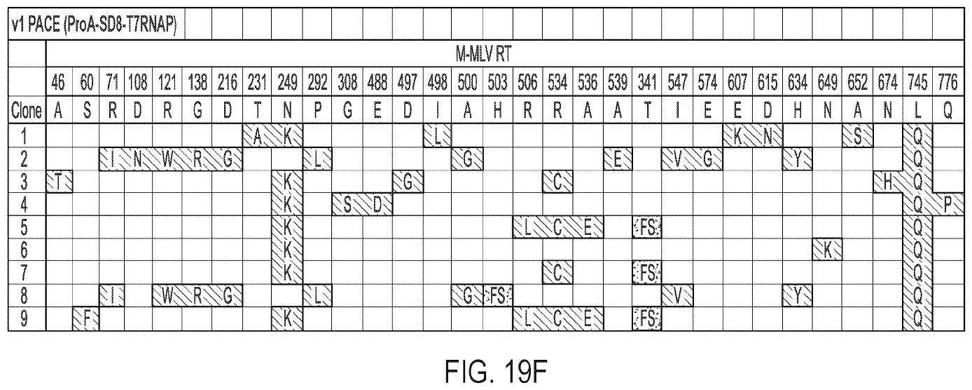

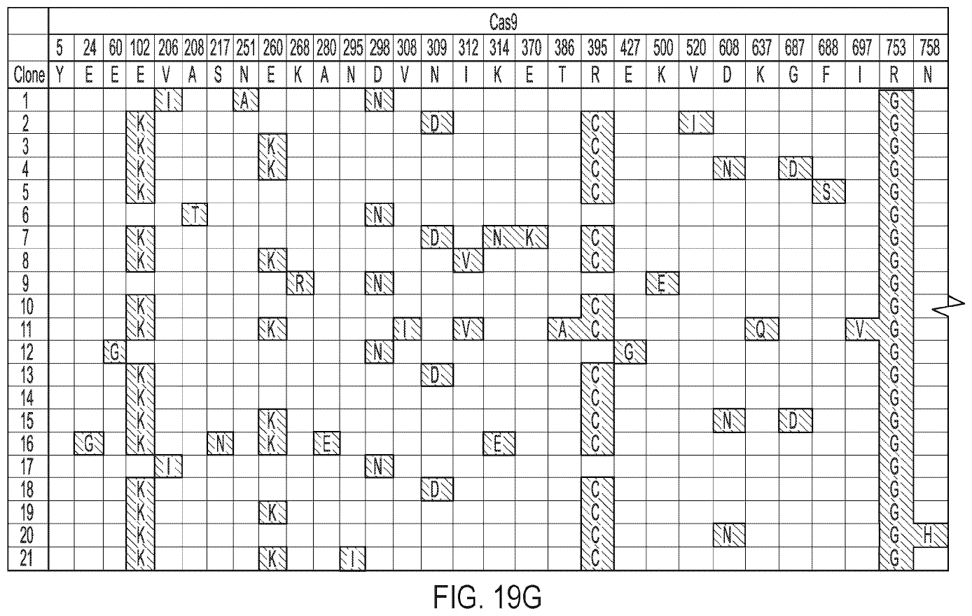

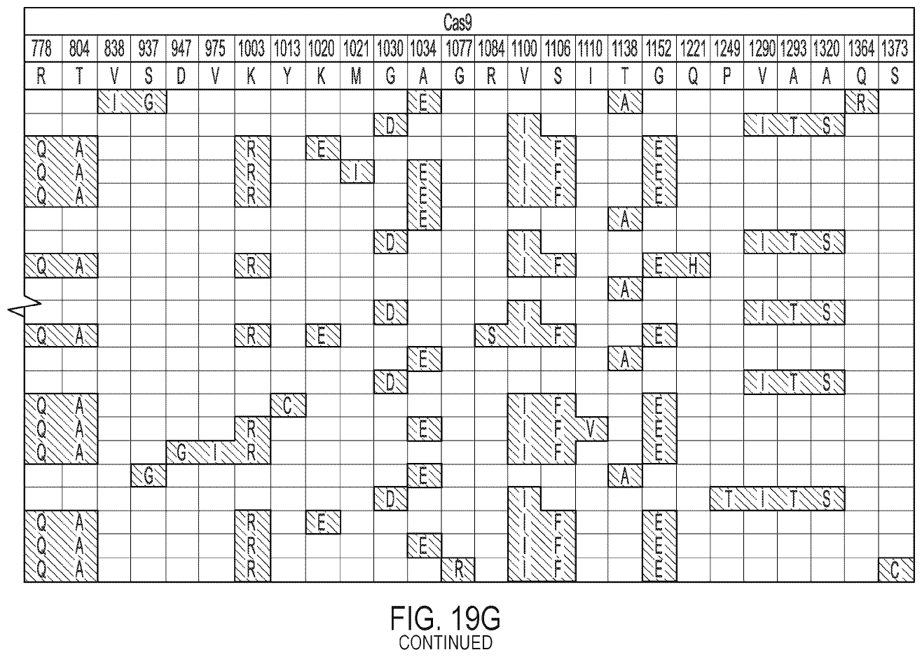

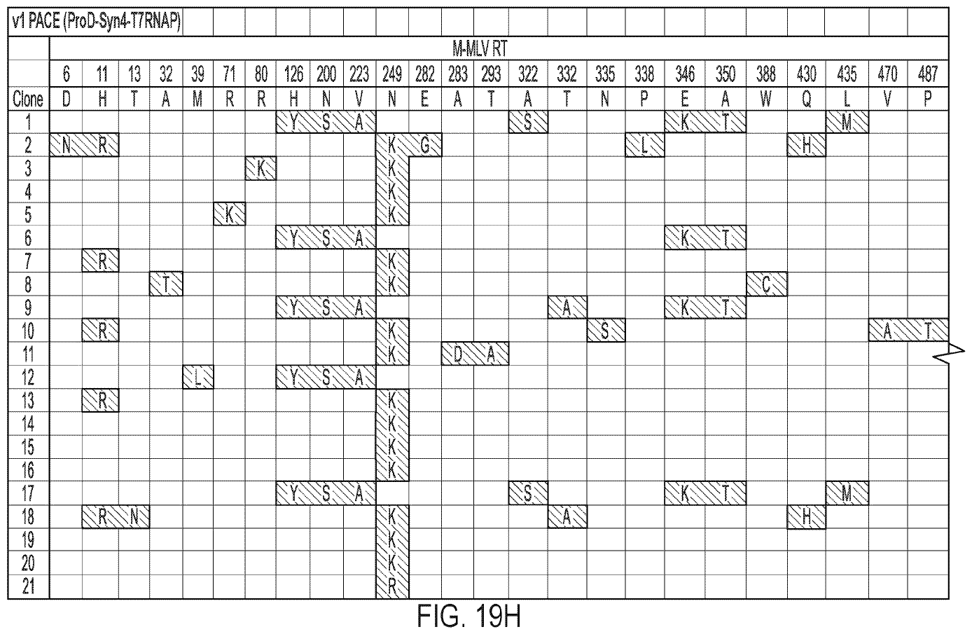

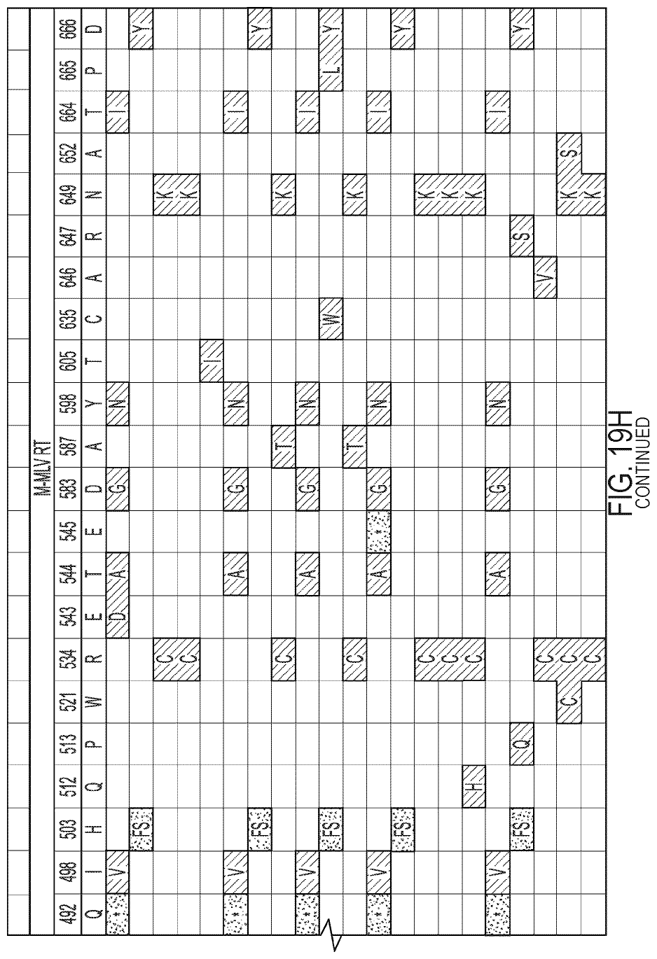

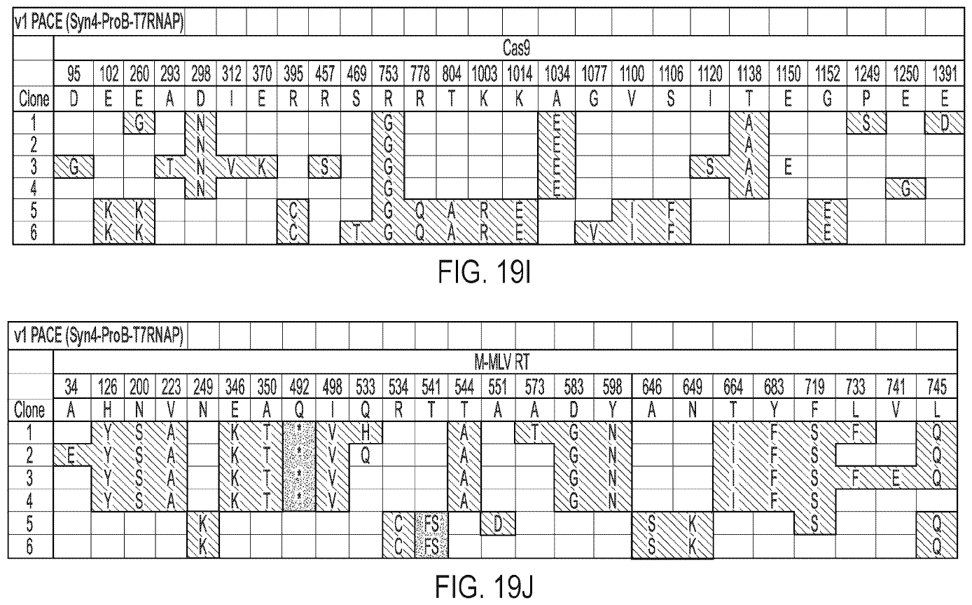

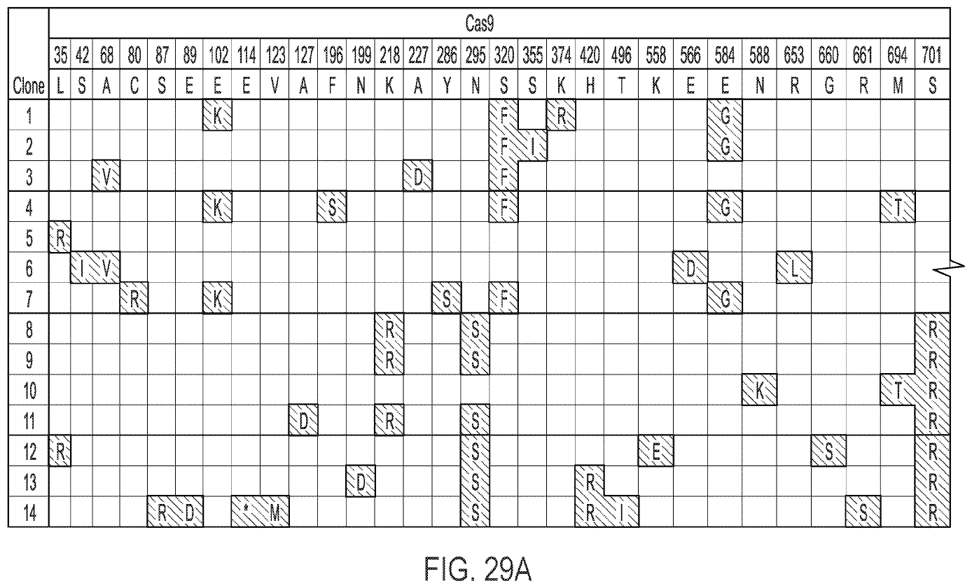

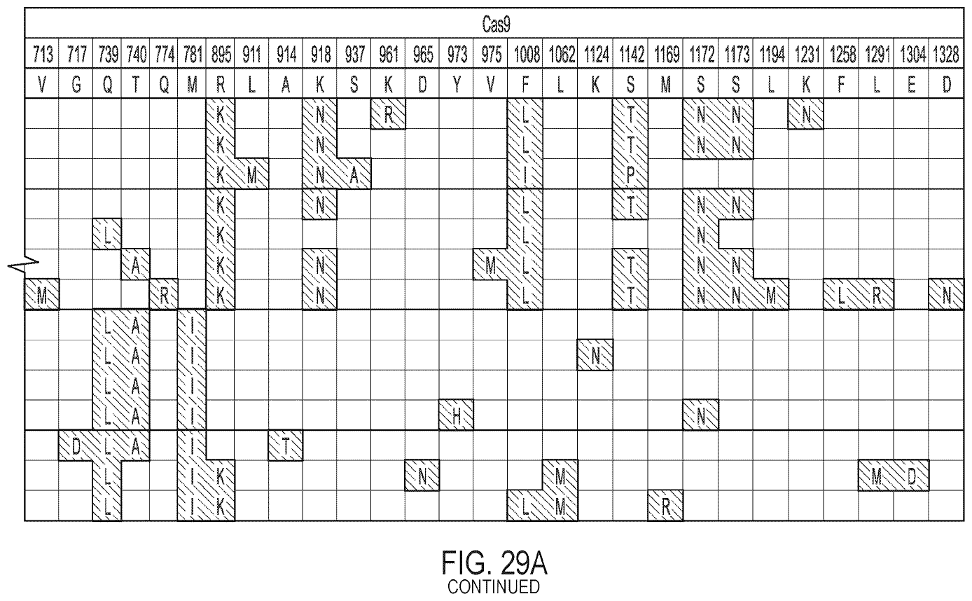

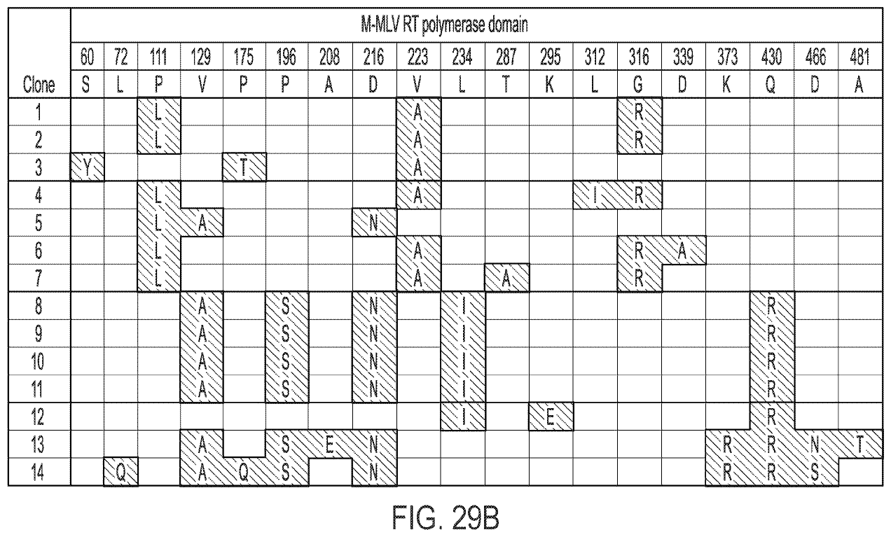

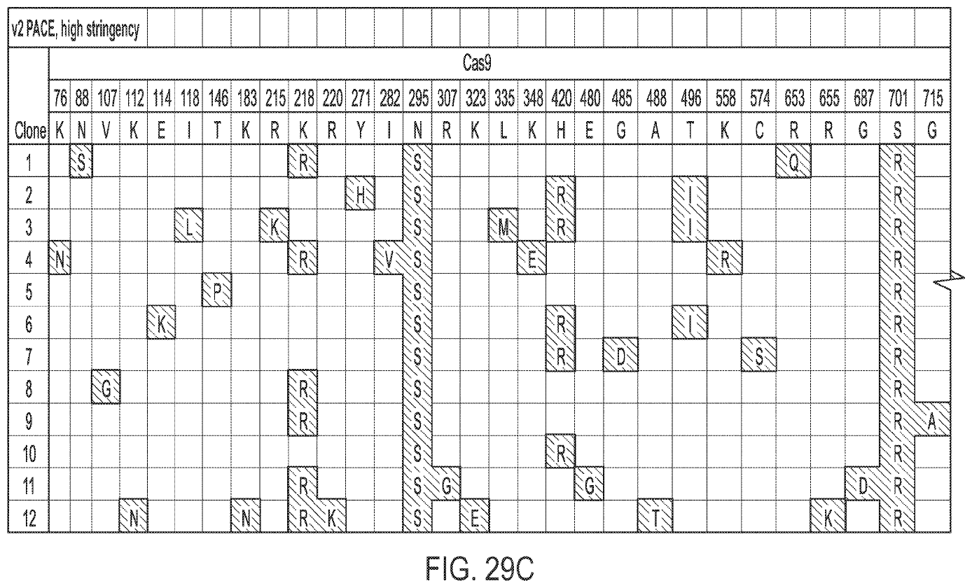

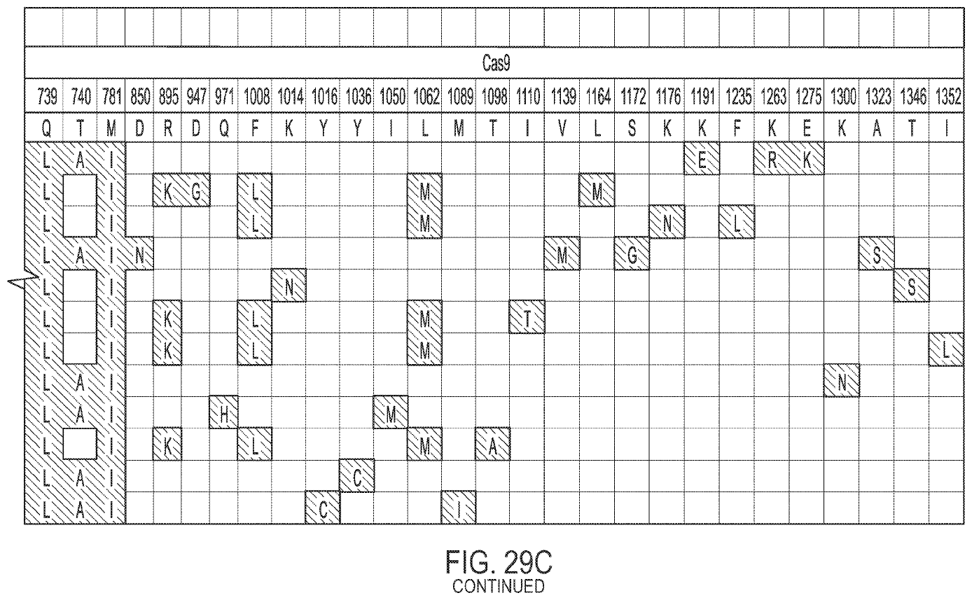

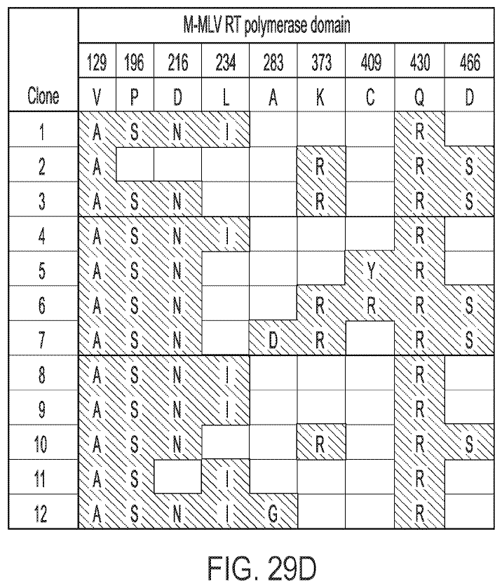

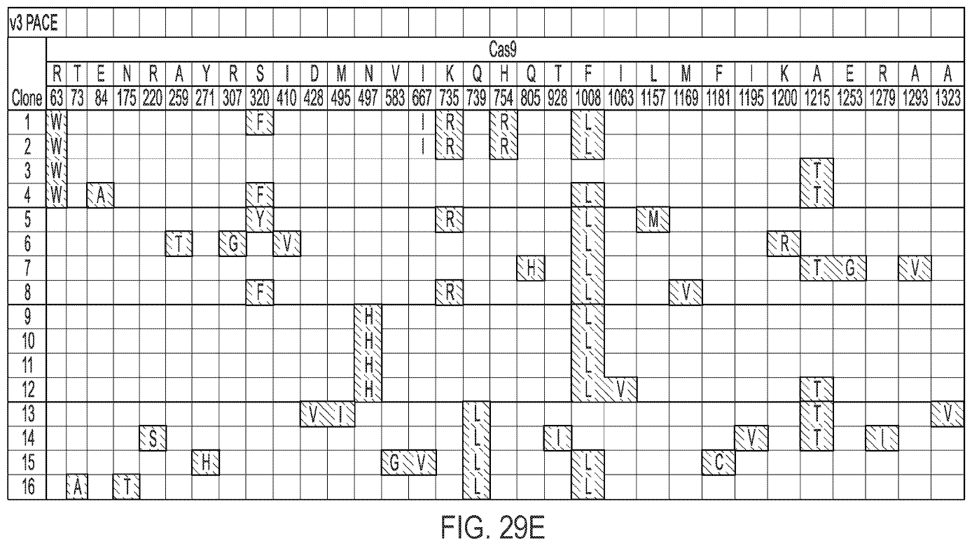

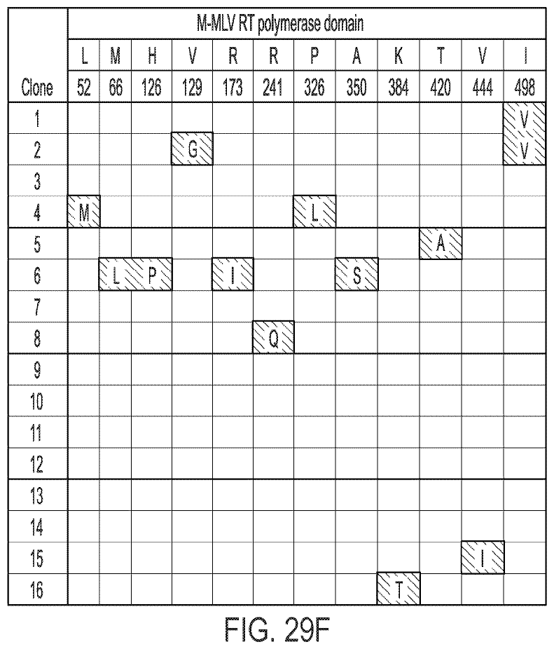

Finally, the fact that during the whole-editor evolutions, the Cas9 domain of the prime editor also acquired dozens of conserved mutations in the v1, v2, and v3 circuits, was revisited (FIG. 6A, FIG. 13A). As noted with the RT domain, the mutations that evolved in the Cas9 domain were dependent on the target used during evolution (FIGS. 19A-19J, FIGS. 29A-29F). Unlike RT mutations, though, the Cas9 mutations that enriched during evolution were distributed across the entire Cas9 protein, without evident hotspots in any domain, primary sequence region, or three-dimensional space.

When evolved Cas9 mutants were tested in HEK293T cells, it was surprisingly found that they displayed markedly lower editing efficiencies compared to PE2. Furthermore, more highly evolved variants evoCas9-5 and evoCas9-6 yielded lower mammalian editing efficiencies than less stringently evolved evoCas9-1 through evoCas9-4 (FIG. 6B). Reversion analysis of evolved Cas9 mutants suggested that a subset of evolved mutations was driving lower mammalian cell editing efficiencies (FIG. 13B). Therefore the effects of 163 individual Cas9 mutations in the PEmaxΔRNaseH architecture on prime editing were dissected at two loci in human and mouse cells: RNF2+5 G to T in HEK293T cells and murine Pcsk9+3 C to G/+6 G to C in N2a cells (FIG. 6C) to identify beneficial and detrimental mutations.

These assays revealed several trends. First, the majority of mutations that most strongly decreased editing efficiency at both mammalian targets (such as K1151E, A1034D, K1003E, and K1014E) have either previously been shown to decrease the affinity of Cas9 for DNA, or are predicted to do so based on structures of the enzyme complexed with its DNA substrate⁵⁰⁻⁵⁴ (FIG. 13C). It was also noted that some PACE-evolved mutations occurred at residues such as M694, K1003, and R1060, which have previously been altered to create high-fidelity Cas9 mutants HypaCas9 and eCas9^{51,54} (FIGS. 29A-29F). These observations indicated that PACE-evolved Cas9 variants may bind DNA more weakly than the wild-type enzyme.

It was hypothesized that during PACE in E. coli, Cas9 binding to a target gene can decrease the expression of that gene through a bacterial CRISPRi mechanism⁵⁵, so high-affinity binding to the corrected T7 RNAP gene after prime editing can lower fitness. Conversely, a weakly binding prime editor that performs the edit and then dissociates more quickly from the target DNA may allow the corrected T7 RNAP gene to be more readily expressed on the rapid timescale needed to survive PACE. In mammalian cells, however, requirements for DNA binding may be more stringent due to lower target site concentrations and competing DNA-binding proteins. Therefore, in mammalian cells, prime editing efficiency may suffer from weaker DNA binding by Cas9.

To confirm the bacterial portion of this hypothesis, E. coli was transformed with plasmids encoding a corrected WT T7 RNAP, the pegRNA used in the v1 circuit, a gIII-luxAB fusion under the T7 promoter, and either a wild-type or K1151E PE2 mutant under the control of an arabinose-inducible promoter. This system allowed for the evaluation of the effect that each editor had on the expression of corrected T7 RNAP by measuring luciferase signal. Compared to uninduced bacteria, strains induced to express PE2 exhibited a 2.8-fold lower luciferase signal. Strains induced to express the K1151E mutant, though, showed no reduction in T7 RNAP expression (FIG. 13D). This result supports a model in which the PE PACE circuit not only selects for prime editing activity, but also selects for reduced Cas9 binding to avoid impeding expression of edited T7 RNAP. This model also implied that PACE-evolved mutations that enhanced prime editing in bacteria, once separated from mutations that disrupt DNA binding, might enhance mammalian cell prime editing.

### Engineering Cas9 Variants for Enhanced Prime Editing

In addition to helping with the identification of a cause of detrimental PACE-evolved mutations, the single-mutant Cas9 assays also identified mutations such as H99R, E471K, I632V, D645N, R654C, H721Y, K775R, and K918A that maintained or modestly increased mammalian prime editing efficiency (FIG. 6C). To create Cas9 variants that more substantially enhance mammalian prime editing efficiency, these individual mutations were combined to generate evolved and engineered Cas9 variants, termed PE6e-g (FIG. 6D). These mutants were compared to their parental PEmaxΔRNaseH across a wider array of editing conditions and target sites in HEK293T cells and N2a cells (FIG. 6D and FIG. 13E). At five of the 13 sites tested, PE6e-g variants improved prime editing efficiency, generating up to a 1.8-fold improvement in editing efficiency compared to PEmaxΔRNaseH. This result demonstrates that evolved and engineered Cas9 variants are capable of improving mammalian prime editing efficiency and suggests that Cas9, not the RT, limits prime editing efficiencies for some edits.

For other edits, though, PE6e-g did not change editing outcomes or even decreased editing efficiencies relative to PEmaxΔRNaseH (FIG. 6D, FIG. 13E). Like for the RT domain of the editor, this site-specificity was forecasted by the sequence-specific enrichment of different mutations during evolution. However, unlike the results concerning the RT domain, a clear relationship was not observed between characteristics of the edit/pegRNA and the benefits of different Cas9 mutants. Nevertheless, the location and nature of the PE6 Cas9 mutations suggest potential explanations for their effect on prime editing. The H721Y mutation is predicted to perturb an interaction between Cas9 and stem loop 2 of the guide RNA scaffold (FIG. 13F), so it is possible that its effects differ based on the specific pegRNA used. Interestingly, the K775R and K918A mutations are located in Cas9's L1 and L2 linkers, which are involved in R-loop stabilization and also mediate conformational changes in the HNH domain upon DNA binding⁵⁶. Furthermore, the K918A mutation has been shown in vitro to abrogate interactions between Cas9 and its R-loop⁵⁷. It is therefore tempting to speculate that the site-dependent impact on prime editing of these Cas9 variants reflects a balance between initial R-loop formation and dissociation from the nicked DNA strand to enable reverse transcription initiation. Future biochemical and library-based studies may illuminate which prime edits would benefit from specific Cas9 mutants. Currently, screening PE6e-g, in addition to the Cas9 domain in PEmax, is recommended when optimizing a prime editing strategy for a site of interest. If only one Cas9 mutant can be tested in addition to the PEmax Cas9, PE6e is the mutant most likely to yield improvements (FIG. 6D).

### Combining PE6 RT and Cas9 Mutants

To maximize prime editing efficiencies, evolved RT variants and evolved Cas9 variants can be evaluated separately and then combined. For example, the size-minimized evolved Ec48 mutant in PE6a exhibits lower editing efficiencies than PEmax at the CXCR4 and IL2RB loci (FIG. 6E), but the evolved Cas9 domain in PE6e improves prime editing efficiency at those loci (FIG. 6D). Combining these two domains (using the naming convention PE6a/e), restores prime editing efficiency to near-PEmax levels, while maintaining the small size of the PE6a RT (FIG. 6E). Additionally, Cas9 and RT domains that both enhance editing efficiency for a particular edit can be combined: both the RT domain of PE6c and the Cas9 domain of PE6g improve twin prime editing efficiency for the recoding exon 4 of the PAH gene. When these domains are combined to generate PE6c/g, the benefits to editing efficiency were additive, yielding a 2.9-fold improvement over PEmaxΔRNaseH (FIG. 6F). These results demonstrate that PE6 RT domains and Cas9 domains can be treated modularly, and that combining evolved domains can overcome deficits in one domain or yield cumulative improvements from both domains.

### Recommendations and Applications of PE6 Mutants

The suite of novel prime editors engineered and evolved herein (PE6a-g) offer improvements in editor size (PE6a and b), reverse transcriptase activity (PE6c and d), and Cas9-mediated editing efficiency (PE6e-g). From this new set of tools, the choice of prime editor variant for a given application is informed by requirements for editor size, as well as characteristics of the desired edit. A general approach for selecting a prime editor variant is summarized below and in FIG. 6G. Briefly, first examining the size constraints on the editor is recommended. When editor size must be minimized, PE6a should be used: PE6a is the smallest prime editor described to date that is able to achieve state-of-the-art editing efficiencies at most loci. If editor size is restricted due to AAV delivery constraints but does not need to be strictly minimized, PEmaxΔRNaseH and PE6b-d should be considered. Choosing between these four enzymes requires the user to analyze the level of secondary structure in their pegRNA. If the target edit uses a pegRNA with a highly structured 3′ extension (NUPACK-predicted free energy of −23 kcal/mol or more stable for the RTT and PBS) or is a twinPE edit, PE6c and PE6d are likely to be optimal. Conversely, if the target edit utilizes a largely unstructured 3′ extension (NUPACK-predicted free energy of folding less stable than −23 kcal/mol), PEmaxΔRNaseH and PE6b, and PE6c should be examined. Finally, if no size constraints exist, PEmax can also be used in addition to the four editors just discussed. PEmax is still a versatile editor that can be effectively used for many applications. However, if an edit requires a short, unstructured reverse transcription template (RTT) and scaffold insertion-derived indel levels are high when using PEmax, PEmaxΔRNaseH, and PE6b should be evaluated in order to reduce indels. Conversely, if an edit is a twinPE edit or a challenging PE edit, PE6c, and PE6d may offer improvements over PEmax. Finally, differences in PE preferences have been observed between M-MLV-derived editors and Tf1-derived editors. If PEmax is unable to yield efficient editing for a given site, PE6b and PE6c are useful alternatives (FIG. 6G). No matter what optimal RT is used, screening Cas9 variants in PE6e-g in combination with the optimized RT can be done if editing efficiency must be improved further (FIG. 6G). As previously stated, the Cas9 mutants in PE6e-g can substantially improve prime editing at some, but not all, sites.

### PE6 Variants Enable Dual AAV-Mediated In Vivo Twin Prime Editing

Finally, using the decision tree in FIG. 6G, a strategy was devised for performing long, structured prime edits in vivo. Dual-AAV systems that enable PE3 editing in the murine brain, liver, and heart have been previously engineered^{4,29-31}. However, when using efficient dual-AAV systems, M-MLV RT must be truncated, deleting its RNaseH domain, in order for the PE protein, pegRNA, nicking RNA, and their regulatory elements to fit within the packaging capacity of two AAVs (~5 kb per virus including ITRs). Because PE6c and PE6d are the same size as PEmaxΔRNaseH but substantially outperform PEmaxΔRNaseH at highly structured edits in cell culture, PE6c and PE6d might enable new classes of edits to be efficiently installed after dual-AAV mediated delivery in vivo.

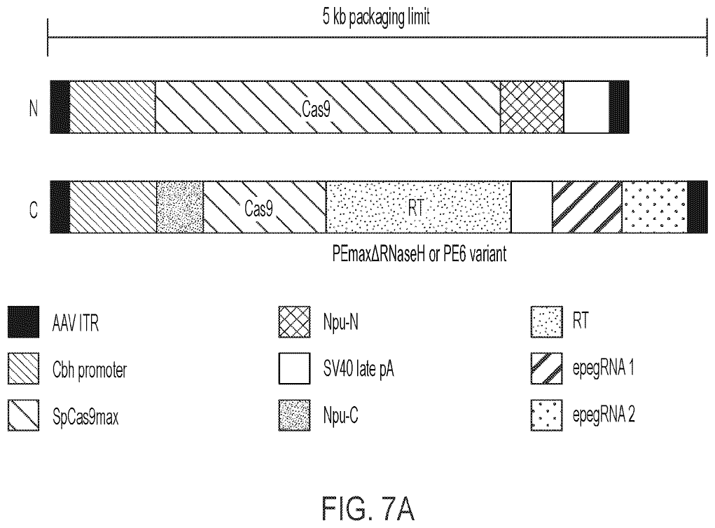

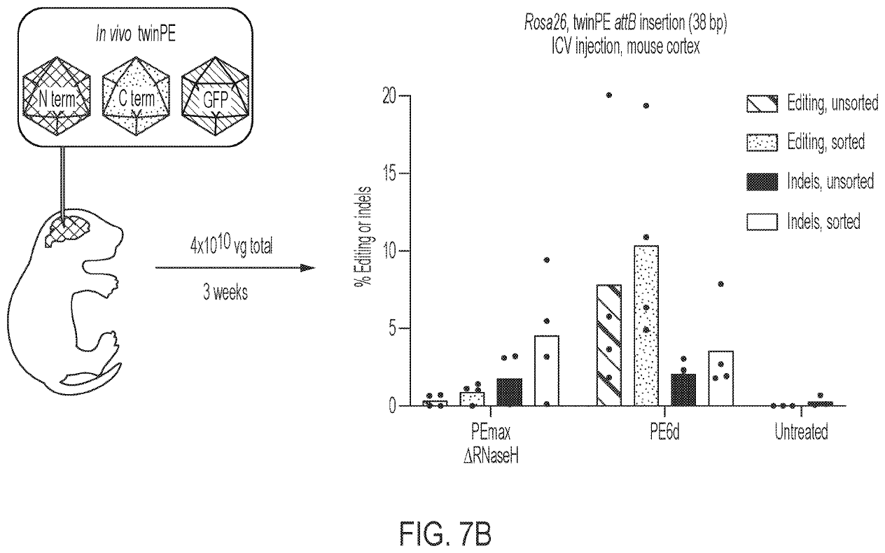

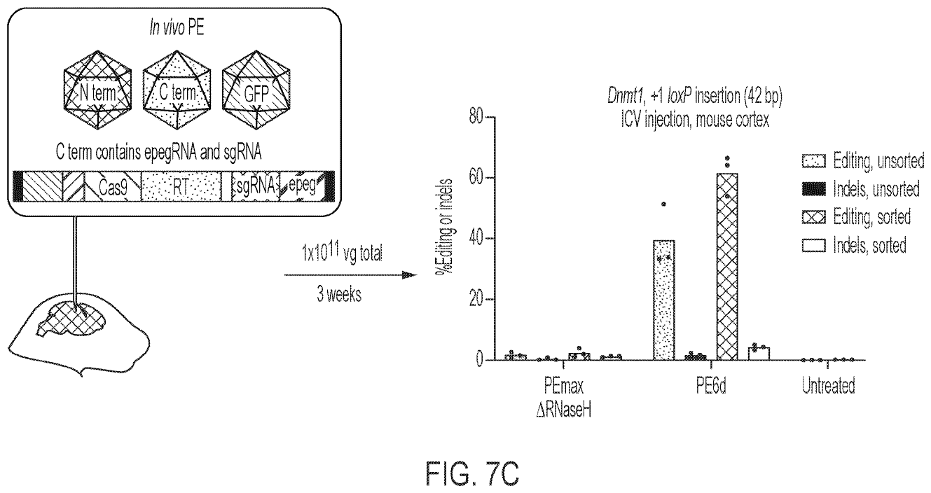

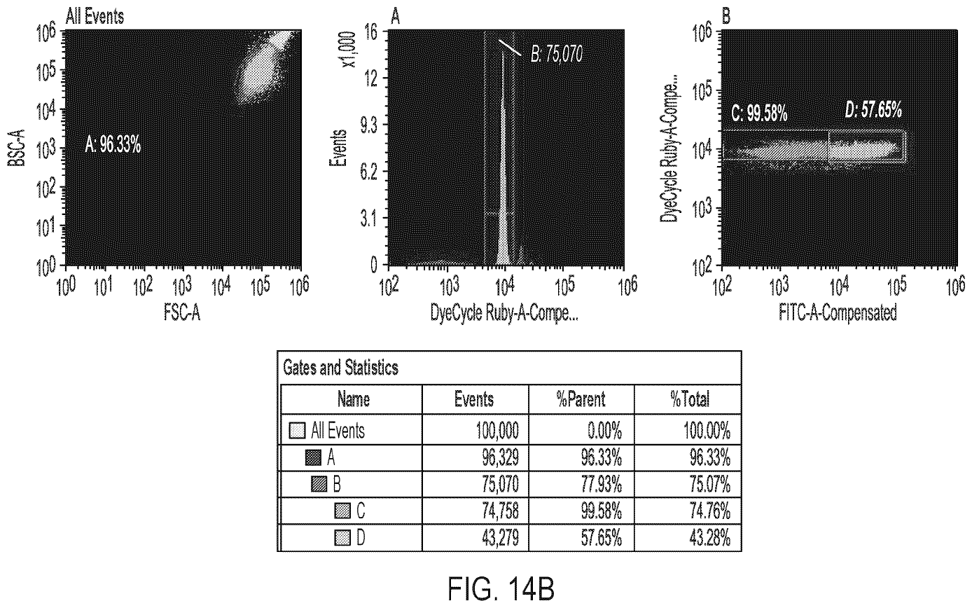

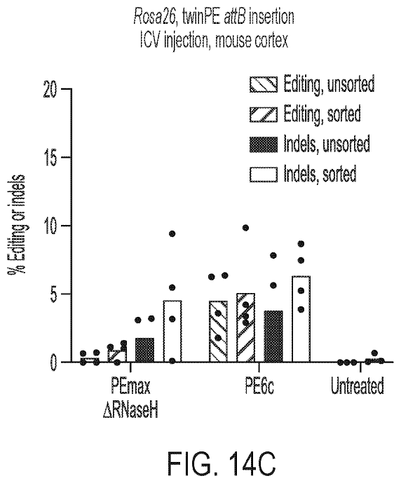

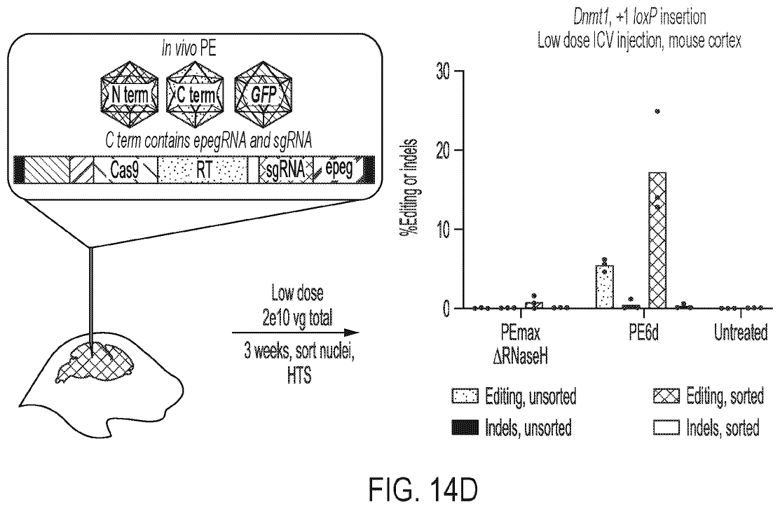

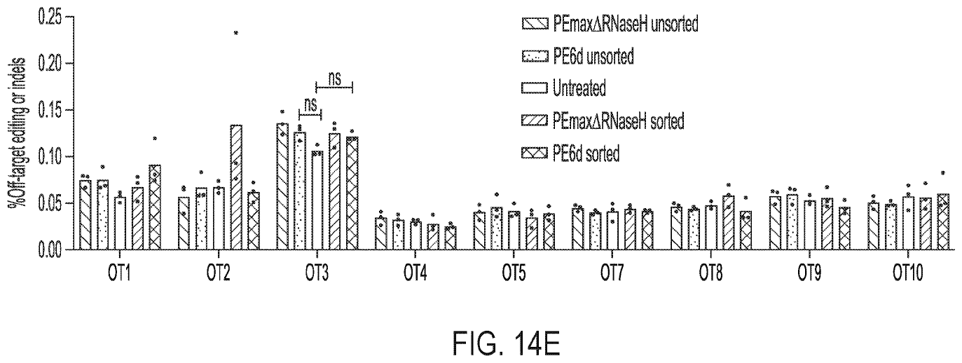

First, PE6 variants were tested to determine if they could enable in vivo dual-flap prime editing. In vivo twinPE has not been previously reported, but it could offer many benefits for applications that require the insertion of several dozens to hundreds of base pairs. To create a dual-AAV system for twinPE (v3em twinPE-AAV), the architecture described in the recently reported v3em PE-AAV prime editor delivery system was used²⁹ (FIG. 7A). In a universal N-terminal AAV, the majority of the Cas9 protein fused to an N-terminal Npu split intein was encoded. In a second C-terminal AAV, a C-terminal Npu split intein fused to the remainder of the prime editor, was encoded using either PEmaxΔRNaseH, PE6c, or PE6d. Notably, further truncation of the Tf1 RT allowed for the minimization of prime editor size an additional 100 bp for this application (FIG. 14A). One modification to the C-terminal virus was made to make it suitable for twinPE: instead of including an epegRNA and nicking guide as originally reported in the v3em PE-AAV system²⁹, two epegRNAs that are required for twinPE were included (FIG. 7A). 10¹⁰ vg of a GFP-KASH virus was also included to mark nuclei from transduced cells. For the target edit, the twinPE-mediated installation of the Bxb1 integrase attB substrate sequence at the murine Rosa26 safe harbor locus was selected. The insertion of recombinase substrate sequences via prime editing into safe harbor loci enables the precise integration of large, gene-sized (>5-kb) DNA into targeted sites in mammalian genomes^{1,10,17,58}.

A low dose of both twinPE AAVs (4×10¹⁰ vg total, 2×10¹⁰ vg per virus) and the GFP virus (1×10¹⁰ vg) were administered via neonatal intracerebroventricular (P0 ICV) injections to C57BL/6 mice. Three weeks later, nuclei from the mice cortices were isolated and bulk (unsorted) or transduced (GFP-positive) nuclei were analyzed (FIG. 14B). Mice treated with PEmaxΔRNaseH AAV showed just 0.34% attB installation in bulk cortex and 0.89% attB installation in transduced cells (FIG. 7B). In comparison, mice treated with PE6 variants showed a marked improvement. PE6c yielded 4.5% and 5.1% insertion of the attB sequence in bulk and sorted nuclei, respectively (FIG. 14C). PE6d demonstrated even more efficient installation, leading to 7.8% and 10.4% editing in bulk and sorted cells, respectively (FIG. 7B). PE6d thus offers an average 23-fold improvement in bulk cortex editing and an average 12-fold improvement in editing efficiency in transduced cells relative to PEmaxΔRNaseH. Notably, this increase in editing efficiency was not accompanied by an increase in indels relative to PEmaxΔRNaseH (FIG. 7B). It is noted that the prime editor AAV dose of 2.7×10¹³ total vg/kg used in this experiment is >4-fold lower than the 1.1×10¹⁴ vg/kg dose used in FDA-approved AAV therapies⁵⁹. These data reinforce that prime editing strategies that were previously intractable in vivo can be achieved using PE6 variants. These data may also represent the first example of in vivo dual-flap prime editing.

### PE6 Variants Enable New Classes of In Vivo Prime Editing Via a Dual-AAV Delivery System

Encouraged by these results, PE6 variants were also tested to determine if they could also mediate improved single-flap prime editing for challenging edits in vivo. To test this, the murine Dnmt1 locus was targeted. All previous in vivo prime edits at this locus were restricted to small genomic changes such as SNPs or small (3-bp) insertions. To test whether PE6 variants can enable large insertions in vivo, the installation of a 42-bp loxP sequence (40-bp loxP plus 2-bp added to preserve reading frame) at this site was investigated, having observed that PE6d outperformed PEmaxΔRNaseH at this edit in cell culture (FIG. 4J). The AAV architecture used for this edit is identical to the previously reported v3em PE-AAV²⁹ and only differed from the twinPE architecture described above by including one epegRNA and one nicking sgRNA, as opposed to two epegRNAs, in the C-terminal virus. For the RT domain in the C-terminal AAV, either PEmaxΔRNaseH or PE6d were used.

Dual AAV was administered via P0 ICV injections and included a GFP-KASH virus to mark nuclei from transduced cells. In this experiment, though, two different viral doses were used: one high dose of 1×10¹¹ vg total (5×10¹⁰ vg per PE virus) and one low dose of 2×10¹⁰ vg total (1×10¹⁰ vg per virus). Three weeks after injection, cortex tissue and isolated nuclei were harvested from either bulk or transduced cells.

In the low-dose condition, loxP insertion in bulk tissue was virtually undetectable when PEmaxΔRNaseH was used, with just 0.03% editing observed on average (FIG. 14D). Sorting for transduced cells improved PEmaxΔRNaseH-mediated editing to 0.75% on average, an efficiency still too low for most applications. Conversely, mice injected with a low dose of PE6d showed substantially improved levels of loxP insertion. In bulk tissue following low-dose treatment, PE6d generated an average of 5.5% loxP insertion, and among transduced cells, the average loxP insertion efficiency was 17% (FIG. 14D). Therefore, in this low-dose condition, PE6d offered a 183-fold increase in bulk editing efficiency and a 23-fold increase in editing levels in transduced cells relative to PEmaxΔRNaseH. PE6d generated just 0.45% indels and 0.25% indels in bulk and transduced cortex, respectively, leading to an editing:indel ratio of 12:1 in bulk cells and 69:1 in transduced cells (FIG. 14D).

In the high dose condition, PEmaxΔRNaseH's editing efficiency improved relative to its low-dose values but remained inefficient, generating just 1.7% and 2.4% loxP installation in bulk and transduced cells, respectively (FIG. 7C). In contrast, PE6d generated very efficient levels of loxP installation when a high AAV dose was used. PE6d achieved an average of 40% and 62% loxP insertion in bulk and transduced cells, respectively (FIG. 7C). Importantly, PE6d-mediated indel levels remained low, at just 1.6% in bulk tissue and 4.2% in transduced cells. These results not only represent a substantial (greater than 23-fold) improvement over PEmaxΔRNaseH in both bulk and transduced cells, but also demonstrate a high editing:indel ratio of 23:1 in bulk cells and 14:1 in transduced cells for PE6d.

Finally, to examine whether the more active RT used in these in vivo experiments increased off-target prime editing, nine of the top ten CHANGEseq-nominated off-target loci were analyzed for the mDnmt1 pegRNA protospacer^{30,60} for the high-dose treated animals (one off-target site did not amplify efficiently by PCR). No off-target editing or indel generation were detected above the background signal from untreated control cells for any of the PEmaxΔRNaseH-treated or PE6d-treated animals. Mean off-target modifications were either less than 0.1% or were not significantly different from the untreated control (p>0.08) (FIG. 14E).

These results demonstrate that while the previous state-of-the-art prime editor PEmaxΔRNaseH cannot support the efficient in vivo installation of difficult, structured PE or twinPE edits, PE6 variants make these changes possible without generating substantial indels or off-target edits.

### Discussion

As presented herein, three key challenges in prime editing were addressed: (1) the development of size-minimized, non-M-MLV RTs that can support state-of-the-art prime editing efficiencies, (2) the development of highly active, dual-AAV compatible RTs, and (3) the development of RT and Cas9 enzymes that offer advantages over PEmax in editing efficiency and/or indel frequencies.

To address the first challenge, PE6a and PE6b were developed. PE6a uses an evolved Ec48 RT that is 810 bp smaller in gene size than the M-MLV RT and 270 bp smaller than the truncated form of M-MLV. PE6a offers a 22-fold improvement over its wild-type counterpart and a 3.7-fold improvement over the size-matched engineered Marathon pentamutant²³ RT in HEK293T cells. These differences are even more pronounced in primary human T-cells. Similarly, PE6b uses an evolved Tf1 RT and is 516 bp smaller in gene size than the M-MLV RT but is able to match or surpass editing efficiencies of PEmax across a variety of sites and conditions. The results presented herein are, to the applicant's knowledge, the first to generate size-minimized, non-M-MLV RTs that can match the prime editing efficiency of PEmax^{4,17,23,24}. Furthermore, these findings suggest that many wild-type RTs can be improved for prime editing through evolution and engineering: the four RTs (Gs, Ec48, Tf1, and M-MLV) evolved in this Example span four different classes of RT (Group II Intron, retron, LTR retrotransposon, and retrovirus, respectively). These results suggest that the techniques and insights from this Example are likely to yield additional useful prime editors when applied at scale to the 80,000 reported reverse transcriptase genes in this important enzyme superfamily.

To solve the second problem of generating highly active, dual-AAV compatible editors, a combination of evolution and engineering were used to produce Tf1-derived PE6c and M-MLV-derived PE6d. By systematically comparing PE6d and PEmaxΔRNaseH, it was discovered that moderately active RTs like PEmaxΔRNaseH are inhibited by RTT secondary structure, and PE6c and PE6d are able to rescue activity when using these structured substrates. The utility of PE6c and PE6d can be predicted by examining the predicted free energy of folding of the pegRNA 3′ extension (excluding the free energy of folding from an epegRNA motif). For highly structured pegRNAs, PE6 variants can offer up to 3-fold improvements in editing efficiency relative to PEmaxΔRNaseH in cell culture. PE6c and PE6d also offer improvements for twinPE editing, even when pegRNAs are not highly structured.

Finally, solving the third problem of exceeding the high performance level of PEmax required many different solutions. First, it was found that PEmax, like the other RTs disclosed herein, is subject to edit-dependent effects of RT processivity. For exceptionally challenging edits such as long twinPE edits, PE6c and PE6d can offer benefits over PEmax.

Conversely, for exceptionally short, unstructured RTTs, indels and scaffold insertion products generated by PEmax can be reduced by using a less active editor such as PEmaxΔRNaseH or PE6b. Second, it was shown that M-MLV-derived RTs and Tf1-derived RTs can offer different advantages. Having another option for the RT domain of the prime editor will likely prove useful for specific therapeutic applications; indeed, the Tf1-derived PE6c editor showed a 4.5-fold improvement over PEmax in the correction of a Bloom Syndrome pathogenic mutation in patient-derived fibroblasts (FIG. 5H). Third, it was demonstrated that evolved and engineered Cas9 domains (PE6e-g) can enhance prime editing efficiencies at some sites and edits. The recommended use cases for PE6 variants are shown in FIG. 6G.

In addition to the PE6 editors themselves, this Example has yielded insights that deepen the understanding of prime editing and inform future improvements. Until this work, patterns among different prime edits and pegRNAs were difficult to discern, even using large library studies³⁹. The RTT-dependent patterns and edit-dependent bottlenecks described in this Example were only discovered after comparing different prime editors; this outcome suggests that differences between prime editors, in addition to differences between prime edits, can be useful when studying prime editing mechanisms.

Finally, the PE6 variants presented here enhance prime editing for a range of therapeutic applications. PE6 variants offer size and efficiency advantages in primary human T cells and patient-derived fibroblasts across a wide variety of target edits and PE systems. Even for nonviral delivery methods in which gene size is not strictly limited, PE6a-d could facilitate critical processes such as the in vitro synthesis of editor mRNA or the packaging of editor proteins into liposomes or engineered virus-like particles⁶³.

Most importantly, PE6c and PE6d enable new classes of prime edits to be effectively installed in vivo via dual-AAV delivery. PE6d was able to achieve 40% editing efficiency for the insertion of a loxP sequence in bulk cortex and 62% loxP insertion in transduced cells. This represents a 23-fold improvement relative to PEmaxΔRNaseH. This improvement is much larger than the 2-fold improvement observed for the same edit in N2a cells; this data suggests that even small benefits generated by PE6 editors in cell culture can manifest as larger improvements in the more difficult scenario of in vivo editing. Furthermore, the raw editing efficiencies achieved by PE6d at this site are high enough to enable systematic tagging of proteins or other basic science applications. In vivo twinPE was also described herein for the first time. Once again, PE6c and PE6d offered order-of-magnitude improvements relative to the previous state-of-the-art editor PEmaxΔRNaseH.

Finally, it is worth noting that both of the in vivo edits demonstrated in this Example involve the insertion of a recombinase recognition sequence. These results lay the foundation for programmable, DSB-free whole gene insertion in the CNS in vivo when paired with the cognate recombinase and donor DNA.

### Methods

### Mammalian Cell Culture Conditions

HEK293T (American Type Culture Collection (ATCC), Cat #CRL-3216), Neuro-2a (N2a from ATCC, Cat #CCL-131) and Huh7 (originated from ATCC) cells were cultured in Dulbecco's Modified Eagle Medium (DMEM) plus GlutaMAX™ (Thermo Fisher Scientific) supplemented with 10% (v/v) fetal bovine serum (FBS) (Thermo Fisher Scientific). Primary Tay Sachs disease patient fibroblast cells were purchased from Coriell Institute (Cat. ID GM00221) and cultured in low-glucose DMEM (Sigma Aldrich) supplemented with 10% (v/v) FBS and 2 mM GlutaMAX™ Supplement (Thermo Fisher Scientific). All cell lines were incubated, maintained, and cultured at 37 °C with 5% CO₂. Cell lines were authenticated by their respective suppliers and tested negative for mycoplasma.

### Generation of HEK293T Models of Tay-Sachs Disease

HEK293T cells homozygous for the HEXA1278TATCins mutation were previously reported¹. HEK293T cells were seeded in a 48-well plate and transfected with 250 ng of a pegRNA plasmid, 83 ng of a nicking sgRNA plasmid, and 750 ng of a PE2-P2A-GFP plasmid programmed to install the HEXA1278TATCins mutation. 3 d after transfection, GFP-positive cells were flow sorted using an LE-MA900 cell sorter (Sony) into a 96-well flat bottom culture well plate. Cells were cultured for 10 d and then analyzed for HEXA1278TATCins mutation installation. Two different clonal, homozygous (100% installation of HEXA 1278TATCins) cell lines were used for experiments.

### Generation of HEK293T Model Cell Lines for Bloom Syndrome, Crigler-Najjar Disease, and Pompe Disease

Pathogenic gene fragments were generated by examining disease alleles from patient-derived fibroblasts in the Coriell Institute database. These gene fragments (300 bp total, flanking the pathogenic mutation) were then ordered as eBlocks (Integrated DNA technologies). These fragments were then cloned into a Sleeping Beauty transposon vector, downstream of a blasticidin resistance gene expression cassette. (The target pathogenic gene itself was not expressed.) 3.2E5 low-passage HEK293T cells were plated in a 6-well dish and transfected with 50 ng of disease allele transposon, 25 ng of transposase, and 725 ng of PUC19 in a total volume of 250 μL using 20 μL lipofectamine 2000 (Thermo Fisher). 48 hours after transfection, cells were trypsinized, resuspended in 2 mL of media, and 60 μL of the resuspended cells were plated in a fresh 6-well plate well with media containing 10 g/mL blasticidin. Cells were passaged until a no-transposase negative control had completely died. The heterogeneous pool of cells was then used for transfection with editors to target the disease allele for correction. In the downstream HTS sample preparation, primers specific for the transposon backbone were used to selectively amplify the knocked-in pathogenic allele, as opposed to the wild-type endogenous allele.

### Isolation and Culture of Primary Human T Cells

Memorial Blood Center (St. Paul, MN) buffy coats were obtained followed by peripheral blood mononuclear cells (PBMC) isolation with Lymphoprep and SepMate tubes (STEMCELL Technologies). CD4+T-cells were purified from PBMCs using the EasySep Human CD4+T Cell Isolation Kit (STEMCELL Technologies). T-cells were cultured in X-VIVO™ 15 Serum-free Hematopoietic Cell Medium (Lonza, Basel, Switzerland) supplemented with: 300 IU/mL IL-2 (PeproTech), GlutaMAX (Gibco), N-acetyl-cysteine (Sigma Aldrich), 5% AB human serum (Valley Biomedical), 50 U/mL penicillin, and 50 μg/ml streptomycin (Gibco).

### General Methods and Molecular Cloning

The following working concentrations were used for antibiotics (Gold Biotechnology): carbenicillin 50 μg/mL, chloramphenicol 25 μg/mL, kanamycin 50 μg/mL, tetracycline 10 μg/mL, streptomycin 25 μg/mL. For all cloning experiments, Nuclease-free water (Qiagen) was used, gene blocks were ordered from Integrated DNA Technologies (IDT), and primers were ordered from either IDT or Eton Biosciences. All synthetic genes were codon-optimized for human cell expression using GenScript's algorithm and obtained as gene blocks from either GenScript or IDT. All plasmid construction was done using Gibson assembly. Briefly, for most Gibson cloning, unless otherwise noted, PCR was done using either Phusion U Green Hot Start II DNA polymerase (Thermo Fisher Scientific) or Phusion Green Hot Start II High-Fidelity DNA polymerase (Thermo Fisher Scientific). The resulting PCR products were purified using QIAquick PCR purification Kit (Qiagen) and fragments were assembled using NEBuilder HiFi DNA assembly master mix (New England BioLabs) according to the manufacturer's protocol. Plasmids for mammalian expression of prime editors were cloned into the pCMV-PE2 vector backbone (Addgene #132775) and plasmids used for the in vitro transcription of different prime editor mRNA were cloned into the pT7-PEmax (Addgene #178113) vector backbone.

Plasmids for the mammalian expression of pegRNAs, sgRNA, and epegRNAs were cloned as previously described³⁷. Briefly, vector backbone expressing a guide RNA under the human U6 promoter was digested using BsaI-HFv2 (New England BioLabs) according to the manufacturer's protocol. The digested fragment was purified by gel electrophoresis with a 1% agarose gel using QIAquick Gel Extraction Kit (QIAGEN). The BsaI-digested vector backbone was then assembled with eblocks ordered from IDT using NEBuilder HiFi DNA assembly master mix (New England BioLabs) according to the manufacturer's protocol.

Vector backbone pU6-pegRNA-GG-acceptor (Addgene, #132777) was used for pegRNA and sgRNA cloning and pU6-tevopreQ1-GG-acceptor (Addgene, #174038) was used for epegRNA cloning. Genotypes of mutants are shown in Table 1. PegRNAs designed to install the 77 pathogenic edits into endogenous sites in HEK293T cells were designed using pegRNA spacer and PBS sequences reported previously⁴⁰.

Fragments assembled after Gibson Assembly were transformed into One Shot Machi cells (Thermo Fisher Scientific) and subsequently plated in 2×YT agar with the appropriate antibiotics. Illustra TempliPhi 100 amplification kit (Cytiva) was used to amplify plasmid DNA before sending it for Sanger sequencing (Quintara Biosciences). Bacterial clones with the verified plasmids were grown in 2×YT media with the appropriate antibiotics. Plasmid DNA used for mammalian cell transfections were isolated using either QIAGEN Plus Midi Kit or Qiagen Plasmid Plus 96 Miniprep Kit while all other plasmids were isolated using QIAprep Spin Miniprep Kit. All isolated plasmid DNA were eluted in nuclease-free water and quantified using NanoDrop One UV-Vis spectrophotometer (Thermo Fisher Scientific).

### Phylogenetic Tree Analysis

RT protein sequences were collected by searching the UniProt database with the BLASTP algorithm. Each individual BLASTP result was filtered to remove duplicate sequences, sequences shorter than 100 residues, and sequences longer than 1000 residues. To reduce phylogenetic complexity, 9-10 representative sequences were randomly sampled from each filtered BLASTP result. Phylogenetic analyses were performed using Geneious Prime. The MUSCLE algorithm was used to generate a multiple sequence alignment of all 543 RT sequences. From this sequence alignment, an unrooted tree was generated using the neighbor-joining tree build method with the Jukes-Cantor genetic distance model.

### Bacteriophage Cloning

Phage cloning was performed in a two-step manner as previously described⁶⁹⁻⁷⁰. Briefly, Gibson Assembly was performed to clone a donor plasmid encoding for either the appropriate reverse transcriptase fused to an Npu C-terminal intein or the entire prime editor protein between two LguI (Life Technologies) type IIS restriction sites. Golden Gate assembly⁷¹ was performed with the donor plasmid along with two other previously reported plasmids (pBT114-splitC and pBT29-splitD) that each encode for one part of a two-part split phage genome. For Golden Gate assembly, all three plasmids were incubated between 30 minutes to 18 hours with LguI enzyme and T4 DNA ligase at 37 °C. Following assembly, the reaction was transformed into chemicompetent S2060⁷² E. coli host cells that contain plasmid pJC175e. This strain is referred to as S2208. Plasmid pJC175e supplies gIII under the phage shock promoter, enabling activity-independent phage propagation. After transformation, the cloned phage was grown overnight in Davis Rich Medium (DRM) at 37 °C with the appropriate antibiotics. Bacteria were then centrifuged for 5 min at 8,000 g and plaqued (see below). Individual plaques were picked and grown in DRM until the culture reached late growth phase. Bacteria were centrifuged and the supernatant containing phage was isolated. Colony PCR was performed and sent for sanger sequencing (Quintara Biosciences) to confirm that the phage encoded for the correct insert.

### Preparation of Chemically Competent Cells

Strain S2060 was used in all experiments. Chemically competent cells were prepared as previously described⁷³. Briefly, an overnight culture of bacteria was diluted 50-fold in 2×YT media with appropriate antibiotics and grown at 37 °C, shaking at 230 RPM until the culture reached an optical density (OD₆₀₀) of 0.4-0.6. Cells were then centrifuged at 4 °C for 10 min at 4,000g. The supernatant was discarded, and the cell pellets were resuspended in ice-cold TSS solution (LB media supplemented with 5% v/v DMSO, 10% w/v PEG 3350, and 20 mM MgCl₂). Resuspended cells were aliquoted, frozen in dry ice and stored at −80 °C until use.

### Phage-Based Luciferase Assay

Phage-based luciferase assays were performed as described previously⁷⁰. For each replicate, one colony of the evolution strain was grown overnight to saturation in DRM and appropriate antibiotics and then back-diluted 50-fold into DRM with appropriate antibiotics. Cultures were grown at 37 °C with shaking at 230 RPM until cultures reached OD₆₀₀=0.4. The mid-log culture was distributed into a 96-well black clear-bottomed plate (Corning), 135 μL of culture per well. 15 μL of high-titer (1×10¹¹ pfu/mL) phage were added to each well. The plate was covered with a breathable seal and incubated, shaking at 37 °C and 230 RPM for 3.5 h. Luminescence and OD₆₀₀ were measured using a plate reader (TECAN). Values reported are OD₆₀₀-normalized luminescence.

### Plasmid-Based Luciferase Assay

Strains for plasmid-based luciferase assays were made by transforming chemicompetent S2060 E. coli with all necessary plasmids, recovering in antibiotic-free DRM for 2 h, and then plating on 2×YT agar containing maintenance antibiotics and 100 mM glucose. For each biological replicate, one colony was picked into DRM and grown overnight. The following day, cultures were back-diluted 50-fold into DRM and antibiotics. For induced samples, arabinose was added to a final concentration of 20 mM. Cultures were grown shaking at 230 RPM and 37 °C for 3 h, after which 150 μL were removed, placed into a 96-well black clear-bottomed plate (Corning), and measured for luminescence and OD₆₀₀ on a plate reader (TECAN). Values reported are OD₆₀₀-normalized luminescence.

### Overnight Propagation Assay

For each replicate, a single colony of a host strain was picked and grown overnight in DRM and appropriate antibiotics. Saturated cultures were back-diluted 50-fold into DRM with appropriate antibiotics and grown for ~2 h, at 37 °C and 230 RPM until OD reached approximately 0.4. For each phage sample, 1 mL of this mid-log culture was placed into a well of a 96-well deep well plate and then infected with 1E5 total phage. Cultures were grown overnight (37 °C and 230 RPM), and then centrifuged for 10 min at 3400g. Supernatant containing phage was collected and then plaqued to determine total number of output phage. Fold propagation is the total number of output phage divided by the number of input phage.

### Plaquing

Plaquing was performed as previously described⁷³. Briefly, a saturated culture of S2208 E. coli was back-diluted 50-fold into DRM containing 50 μg/mL carbenicillin. 2 h later, the mid-log culture (OD=~0.5) was used for plaquing. For each phage to be plaqued, three 100-fold serial dilutions of the sample were made using DRM. 10 μL of the original concentrated sample or each serially diluted sample was combined with 100 μL of mid-log 2208 culture. Immediately after mixing the bacteria and the phage, 1 mL of top agar (2:1 ratio of 2×YT media:2×YT agar, stored at 55 °C until use) was added to the phage/bacteria solution, mixed quickly, and then immediately plated on 2×YT agar plates containing no antibiotics and 0.04% Bluogal (Gold Biotechnologies). The following day, the number of blue plaques were counted for whichever dilution (either the concentrated sample or one of the 100-fold dilutions) gave a discernable number of blue plaques. This number was then used to calculate the concentration of the phage sample in pfu/mL. For cases where activity-dependent plaquing was used, the relevant selection strain replaced S2208s.

### Phage-Assisted Noncontinuous Evolution (PANCE)

To perform one passage of PANCE, chemicompetent selection strains were transformed with MP6³⁶, recovered for 2 h in DRM without antibiotics, and then plated on 2×YT agar plates containing maintenance antibiotics for the selection strain, 25 μg/mL chloramphenicol, and 100 mM glucose. The following day, ~10 colonies were selected from the plate, pooled in DRM containing 25 μg/mL chloramphenicol and maintenance antibiotics, and grown to OD 0.5. Arabinose was then added to the mid-long culture to reach a final concentration of 20 mM to induce MP6 expression. Immediately after addition of arabinose, 1 mL of this culture per PANCE replicate was infected with 1E5 pfu of phage and then incubated in a 37 °C shaker at 230 RPM overnight. The following day, cultures were centrifuged for 10 min at 3400g, and the supernatant containing propagating phage was collected and used to infect the next round of evolution. Phage titer after each round was determined using qPCR (see below),

Typically, 20 μL of phage were used to infect the next round of evolution (a 1:50 dilution). If phage titers were exceptionally high (1E7 PFU/mL or greater), then a 1:100, 1:200, or 1:1000 dilution factor was used instead. If titers were exceptionally low (less than 1E5 PFU/mL), a passage of drift was performed. For drift passages, 2208s containing MP6 were used instead of selection strains. In drift passages, phage were only allowed to propagate for 6-8 h instead of overnight to minimize recombination-mediated cheating. Once a noticeable change in phage propagation in the selection strain occurred, phage were plaqued using 2208s or the selection strain. Individual plaques were then amplified by PCR using primers JLD 1311 and JLD 1313 and submitted for Sanger sequencing to generate inputs for Mutato analysis (hub.docker.com/r/araguram/mutato).

qPCR Determination of PANCE and PACE Titers

Phage titers in PANCE were estimated using qPCR as previously described⁷³. For each qPCR titer experiment, in addition to phage pools from evolution, a standard phage sample of a known high titer (1×10¹⁰ pfu/mL as determined by plaquing) was treated identically to create a standard curve. To titer a phage sample, eight serial ten-fold dilutions of phage were made into DRM (no antibiotics). 25 μL of each serial dilution was heated to 80 °C for 30 min. Then 5 μL of heat-treated phage was combined with 44.5 μL of 1× DNase buffer and 0.5 μL of DNase (NEB). The DNase mixture was heated to 37 °C for 20 min and then 95 °C for 20 min to remove genomes from replication-incompetent polyphage. 1.5 μL of the heat-inactivated DNase mixture was pipetted into a 28 μL Q5 High-fidelity PCR reaction (NEB) containing SYBR Green (Invitrogen) and primers M13-fwd and M13-rev. qPCR was run on a Biorad CFX96 Real Time system with the following cycling conditions: 98 °C for 2 min, [98 °C for 10 s, 60 °C for 20 s, 72 °C for 15 s]×40. Cq values for phage of known titer were used to generate a standard curve, and other samples' Cq values were used to calculate phage titer in pfu/mL.

### Phage-Assisted Continuous Evolution (PACE)

Chemicompetent selection strains were transformed with MP6, recovered for 2 h in DRM without antibiotics, and then plated on 2×YT agar plates containing maintenance antibiotics for the selection strain, 25 μg/mL chloramphenicol, and 100 mM glucose. The following day, colonies were picked into DRM and appropriate antibiotics into wells of the top row of a deep well 96-well plate and serially diluted 5-fold down the rows of the plate. The plate was incubated shaking at 37 °C and 230 RPM overnight. The next day, wells with an OD₆₀₀ between 0.1 and 0.9 were pooled, diluted to a total volume of 140 mL in DRM and maintenance antibiotics and grown (37 °C, 230 RPM) until OD₆₀₀ reached 0.5. This culture was used to fill an 80 mL chemostat and four 15-mL lagoons.

The filled chemostat and lagoons were inserted into a PACE apparatus. Configuration of the PACE apparatus was identical to previously described setups⁷³. The flow rate for the chemostat was controlled by a Masterflex L/S Digital Drive Pump (Cole-Parmer) using a Masterflex L/S Multichannel pump head. Supplement solution for a PACE carboy was made with 500 mL DI water, 59 g Harvard Custom Media C, 50 μL of 0.1 M CaCl₂, 120 μL of a trace metal solution, 400 mg chloramphenicol pre-dissolved in 3 mL of ethanol, and appropriate maintenance antibiotics for the selection strain (500 ng carbenicillin, 1 g spectinomycin, and 300 mg kanamycin, as needed depending on the PACE strain). The supplement was then combined with a 20 L solution of Harvard Custom Media A to create PACE media. This final media was used as input into the chemomstat. The 80 mL chemostat was maintained at OD=~0.5, starting with a flow rate of approximately 80 mL/h. The chemostat's effective flow rate (vol/h) was adjusted throughout the PACE experiment to maintain a constant OD₆₀₀, either by increasing the flow rate on the pump or by decreasing the chemostat volume by lowering the waste needle. Chemostat waste was collected in a carboy containing bleach. Lagoon flow rates were also controlled by a Masterflex L/S Digital Drive Pump (Cole-Parmer) using a Masterflex L/S Multichannel pump head. Mid-log culture from the chemostat was used as the input for all lagoons, and lagoon waste was collected in a carboy containing bleach. To achieve MP6 induction in the lagoons but not the chemostat, arabinose was continuously added to each lagoon. 250 mM arabinose was taken up into a 50 mL syringe, and using a six-channel programmable syringe pump (New Era NE-1600), arabinose was pumped into each lagoon (0.6 mL/h of arabinose for a 15 mL/h lagoon flow rate). The PACE apparatus was allowed to equilibrate for 1-12 hours before phage infection.

To begin the PACE, all pumps were turned off, and a total of 1.5E8 pfu were injected into each lagoon. After 10 minutes, pumps were turned back on, and ~400 μL was removed from each lagoon for the t=0 timepoint. Lagoon flow rates began at 0.5 vol/h. Subsequent timepoints were taken every 8-24 hours, and each phage sample was stored at 4 °C after removal from the lagoon. Immediately after sample collection, lagoon titers were measured using qPCR. If titers were the same as or higher than the previous timepoint, the flow rate was increased by 0.5 vol/h, and arabinose pump rates were adjusted accordingly. If titers were decreasing, flow rate was held constant. Plaquing was used to determine more accurate titers for reporting in figures.

At the end of the PACE experiment, phage were plaqued in two different strains to check for cheating (S2060s to check for gIII recombinants and S2060s transformed with a pT7-gIII plasmid one to check for T7 recombinants), and amplified by PCR to check for bands corresponding to typical cheater recombinants using primers JLD 1311 and JLD 1313. If cheating was not detected (i.e., no plaques on cheater strains and no additional bands via PCR), phage were plaqued in either 2208s or the selection strain. Individual plaques were then amplified by PCR and submitted for Sanger sequencing to generate inputs for Mutato analysis. (hub.docker.com/r/araguram/mutato).

### Transfection of HEK293T, N2a, and Huh7 Cells

All transfections used to evaluate editors in mammalian cells were performed in TC-treated 96-well plates (Corning). For both HEK293T cells and N2a cells, a T-75 flask of cells was washed with PBS, trypsinized using TrypLE Express enzyme (Thermo Fisher Scientific), and diluted to a concentration of 1.6E5 cells/mL in DMEM (10% FBS, no antibiotics). 100 μL of diluted cells were added to each well of a 96-well plate. 18-24 h after plating, cells were transfected. For unmodified HEK293T cells, the following conditions were used: 100 ng editor, 40 ng of pegRNA, and 13 ng nicking sgRNA (or, if conducting a twinPE experiment, 40 ng of the other pegRNA) plasmid were combined in a total volume of 6.25 μL Opti-MEM (Thermo Fisher Scientific) per well. For each well, 0.5 μL of Lipofectamine 2000 (Thermo Fisher Scientific) was mixed with 5.75 μL OptiMEM and then combined with the DNA mixture. 10 min later, the DNA/lipid mixture was added dropwise to cells.

For the HEK293T Tay Sachs model cell line, the following conditions were used: 200 ng editor, 40 ng pegRNA, 13 ng nicking sgRNA.

For N2a cells, the procedure was the same as HEK293T cells, except the plasmid DNA amounts differed: for PE3, 175 ng editor, 50 ng pegRNA, and 20 ng nicking sgRNA (or, if conducting a twinPE experiment, 50 ng of the other pegRNA) were used. For PE5 experiments in N2as, 100 ng of MLH1dn plasmid was added.

For the twinPE transfection performed in Huh7 cells, 150,000 cells were plated in poly-D-lysine-coated 24-well plates (Corning) in DMEM plus GlutaMAX supplemented with 10% FBS. After 16-24 hours, cells were transfected with 400 ng of prime editor plasmid DNA, and 40 ng of each pegRNA plasmid DNA with 2 uL Lipofectamine 2000 (Thermo Fisher Scientific), according to the manufacturer's protocol.

### HTS Sample Preparation

72 h following transfection, cells were washed with PBS (Thermo Fisher Scientific) and lysed for 1 h at 37 °C in lysis buffer (10 mM Tris-HCl pH 8, 0.05% SDS and 25 μg/mL proteinase K (Thermo Fisher)). Lysate was then heat inactivated at 80 °C for 30 min. 1 μL of lysate was used as an input for PCR1. PCR1 reactions were 25 μL total, using the Phusion Hot Start II kit (Thermo Fisher), 0.75 μL of DMSO, and 0.125 μL of each 100 μM primer. PCR1 was performed under the following cycle conditions: 98 °C for 3 min, [98 °C 15 s, 61 °C 30 s, 72 °C 30 s]×29, 72 °C 2 min. Exceptions to these cycling conditions include: N2a sites Pcsk9 and Dnmt1 used an annealing temperature of 70 °C instead of 61 °C, and for twinPE edits, 25 cycles were performed as opposed to 29, in order to decrease PCR bias.

Samples were barcoded in a second PCR reaction (PCR 2). PCR2 reactions were 25 μL total, using the Phusion Hot Start II kit (Thermo Fisher Scientific), 1.25 μL each of 10 μM Illumina barcoding primers, and 1 μL of PCR1. All PCR2 reactions were performed using the following cycling conditions: 98 °C for 3 min, [98 °C 15 s, 61 °C 30 s, 72 °C 30 s]×8, 72 °C 2 min. After PCR2, samples of similar lengths were pooled and gel extracted in a 1% agarose gel using a Qiaquick gel extraction kit (Qiagen). Concentrations of purified libraries were determined using a Qubit double-stranded DNA high sensitivity kit (Thermo Fisher Scientific) according to the manufacturer's instructions. Libraries were diluted to 4 nM and sequenced using a Miseq (Illumina) using an Illumina Miseq v2 Reagent kit or an Illumina Miseq v2 Micro Reagent kit using single read cycles.

### HTS Analysis

Samples were demultiplexed with Miseq Reporter (Illumina). CRISPResso2 was used to analyze demultiplexed reads. For samples in which the prime edit was a single base change, samples were aligned to the wild type amplicon in batch mode, using the following parameters: “-q 30”, “-discard_indel_reads TRUE”, and “-qwc”. The value of the qwc parameter, which defined the portion of the sequence to be analyzed for indels, differed for each amplicon. The qwc interval included 10 bp before the first nick of the amplicon (whether that was the prime editing nick site or the PE3 nicking guide nick site) to 10 bp after the second nick of the amplicon (whether that was the prime editing nick site or the PE3 nicking guide nick site). To calculate percent editing, the percent base change was multiplied by an indel correction factor. Percent base changes were found in the CRISPResso2 output file titled “Reference.Nucleotide_percentage_summary.txt”. The indel correction factor was obtained by dividing “reads aligned”/“reads aligned all amplicons” values in the “CRISPResso_quantification_of_editing_frequency.txt” CRISPResso2 output file. To calculate percent indels, “Discarded” was divided by “reads aligned all amplicons” in the same file.

For samples in which the prime edit was multiple base changes or an insertion or deletion, CRISPResso2 was run in HDR batch mode. Parameters were identical to those described above for single nucleotide changes, but an additional parameter “e” was included, the value of which was the sequence of the desired, edited amplicon. For these types of edits, percent editing was calculated by dividing the HDR-aligned reads/reads aligned all amplicons and then multiplying by 100. Indels were calculated by adding the “Discarded” reads from the reference-aligned sequences and the “Discarded” reads from the HDR-aligned sequences and then dividing that sum by “reads aligned all amplicons”. All of these values are found in the “CRISPResso_quantification_of_editing_frequency.txt” file when HDR mode is used.

To quantify scaffold integration, a custom python script provided below was used. From top to bottom, the sequences are SEQ ID NOs: 69-74. For each condition, scaffold integration is the percentage of (number of amplicons with scaffold-templated bases)/(number of reads that align to the amplicon).

Custom python script used for quantification of pegRNA insertions at the target
genomic locus (First reported in Anzalone et al., 2019)

## sgRNA scaffold sequence search ##
import pandas as pd
import Bio as bio
from Bio import SeqIO
import glob
#generates list of fastq files to analyze
sources = glob.glob(′*.fastq′)
#reads the fastq files into a dictionary with the file names as keys
fastqdict = { }
for i in range(len(sources)):
temp = list(SeqIO.parse(sources[i], ″fastq″))
fastqdict[sources[i]]= [str(temp[k].seq) for k in range(len(temp))]
#the referenced sequence to be searched for is entered into the following dictionary with
#an appropriate key
scaffdict =
{′RNF2′:CTGATGTGTTCGTTGCACCGACTCGGTGCCACTTTTTCAAGTTGATAAC
GGACTAGCCTTATTTTAACTTGCTATTTCTAGCTCTAAAACCAGGTAATGACTA
AGATGAC′,
′PRNP′: ′TGGCGTCTACATGCACCGACTCGGTGCCACTTTTTCAAGTTGATAACGG
ACTAGCCTTATTTTAACTTGCTATTTCTAGCTCTAAAACCCAAGGCCCCCCACC
ACTGC′,
′FANCF′: ′ACCTTGATCGCTTTTCCGCACCGACTCGGTGCCACTTTTTCAAGTTGA
TAACGGACTAGCCTTATTTTAACTTGCTATTTCTAGCTCTAAAACGGTGCTGCA
GAAGGGATTCC′,
′EMX1′: ′GAAGTGCTCCCATCACGCACCGACTCGGTGCCACTTTTTCAAGTTGATA
ACGGACTAGCCTTATTTTAACTTGCTATTTCTAGCTCTAAAACTTCTTCTTCTGC
TCGGACTC′,
′VEGFA′: ′TGAGTGCTCCAGATGGCACATTGCACCGACTCGGTGCCACTTTTTCAA
GTTGATAACGGACTAGCCTTATTTTAACTTGCTATTTCTAGCTCTAAAACTCAT
CTGGCCTGCAGACATC′,
′Ctnnb1′: ′TCAGGAAAGGAGCGCACCGACTCGGTGCCACTTTTTCAAGTTGATAAC
GGACTAGCCTTATTTTAACTTGCTATTTCTAGCTCTAAAACTGAGTGGCAAGGG
CAACCCTC′,
}
#matches and counts iterative slices of the reference string to the appropriate fastq files
#reference key must be contained in the name of the fastq file
#generated values represent cumulative counts for a minimum degree of sgRNA
integration
#i.e. a given value x means x reads contain y or more bases of the scaffold
resultdict = dict.fromkeys(sources)
for key in fastqdict:
for scaffold in scaffdict:
if scaffold in str(key):
resultlist = []
for j in range(len(scaffdict[scaffold])):
extent = scaffdict[scaffold][0:(j + 1)]
counter = 0
for i in range(len(fastqdict[key])):
if extent in fastqdict[key][i]:
counter = counter + 1
resultlist.append(counter)
resultdict[key] = resultlist
#writes the results into a dataframe indexed from 1
resultdf = pd.DataFrame.from_dict(resultdict)
resultdf = resultdf.reindex(sorted(resultdf.columns), axis = 1)
resultdf.index = range(1,len(resultdf)+1)
#converts the cumulative count values into specific counts
#i.e. a given value x means x reads contain exactly y bases of the scaffold
resultdf2 = resultdf.copy()
for entry in resultdf:
for i in range(1,len(resultdf[entry])+1):
try:
resultdf2[entry][i] = resultdf[entry][i]-resultdf[entry][i + 1]
except:
resultdf2[entry][i] = resultdf[entry][i]
#converts the specific counts values into frequencies
resultdf3 = resultdf2.copy()
for entry in resultdf3:
resultdf3[entry] = resultdf2[entry].div(resultdf[entry][1])*100
#reads the results into excel files
resultdf.to_excel(′cumulativecounts.xlsx′)
resultdf2.to_excel(′specificcounts.xlsx′)
resultdf3.to_excel(′specificfrequencies.xlsx′)

In Vitro Transcription (IVT) of Editor mRNA

IVT of editor mRNA was performed as described previously³⁷. Editors were cloned into pT7 expression constructs (example Addgene 178113). To generate linear DNA templates for IVT, the pT7-editor plasmids were amplified by PCR using the Phusion U green multiplex master mix (NEB) using primers IVT-fwd and IVT-rev. PCRs were purified using the QIAquick PCR purification kit (Qiagen) and eluted in water. IVT reactions were performed using a T7 high yield RNA synthesis kit (NEB), following the manufacturer's directions with two exceptions: Trilink's CleanCap reagent AG was added, and the uridine 5′ triphosphate in the kit was replaced with N¹-methylpseudouridine 5′ triphosphate (Trilink). Each 160 μL reaction used 8 μL 10× reaction buffer, 8 μL 100 mM ATP, 8 μL 100 mM CTP, 8 μL 100 mM GTP, 8 μL 100 mM N¹-methylpseudouridine 5′ triphosphate, 6.4 μL 100 mM CleanCap AG, 16 μL T7 RNAP mix, and 1 microgram of purified linear template DNA. After assembly, reactions were incubated at 37 °C for 4 h. Samples were then DNase treated by adding 544 μL water, 80 μL DNase reaction buffer (NEB), and 60 μL DNaseI (NEB) to the IVT reaction. Samples were incubated at 37 °C for 15 min, and RNA was purified using a lithium chloride precipitation, followed by two washes in 70% ethanol. RNA was resuspended in nuclease-free water, and purity and quality were verified using a 2% agarose gel stained with SYBER Gold (Thermo Fisher Scientific). RNA was stored at −80 until use.

### Electroporation of Patient-Derived Fibroblasts

An 80% confluent T-75 flask of patient-derived fibroblasts (Coriell) was washed with PBS (Thermo Fisher Scientific), trypsinized using TrypLE Express enzyme (Thermo Fisher Scientific), and suspended in 10 mL of media. The following media was used for each patient-derived fibroblast line: low-glucose DMEM (Sigma Aldrich) supplemented with 10% (v/v) FBS and 2 mM GlutaMAX™ Supplement (Thermo Fisher Scientific) for Tay Sachs Disease (ID: GM00221), high-glucose DMEM (Thermo Fisher Scientific) supplemented with 15% (v/v) FBS and 2 mM GlutaMAX™ Supplement (Thermo Fisher Scientific) for Pompe Disease (ID: GM20092) and EMEM (ATCC) supplemented with 15% (v/v) FBS for both Crigler-Najjar Syndrome (ID: GM09551) and Bloom Syndrome (ID: GM02085). Cells were transferred to falcon tubes and centrifuged for 5 min at 150 g. During centrifugation, RNA reagents were prepared. For each sample, 1 μL of 1 μg/μL editor mRNA was added to a PCR tube, along with 0.45 μL of a 200 μM HEXA1278ins correction pegRNA solution and 0.6 μL of a 100 μM HEXA1278ins correction nicking sgRNA solution. An SE cell line kit (Lonza) was used to perform electroporation. 90.2 μL of SE nucleofector solution was mixed with 19.8 μL of supplement solution to make reconstituted Lonza buffer. Pelleted cells were washed with PBS and resuspended in the reconstituted Lonza buffer. 20 μL of resuspended cells was added to each editor/epegRNA/nicking guide mixture, transferred to a cuvette (Lonza), and electroporated using program CM130 on a Lonza 4D nucleofector with X unit (100,000 cells per electroporation condition). Immediately after electroporation, 80 μL of media was added to each well and incubated at room temperature for 10 min. 1 mL of media was aliquoted into each well of a 24 well plate, and all cells were transferred to this plate. Cells grew for 5 days, with a media change at day 3, before lysis and sequencing.

### Electroporation of Primary Human T Cells

T cells were cultured in X-VIVO™ 15 Serum-free Hematopoietic Cell Medium (Lonza, Basel, Switzerland) supplemented with: 300 IU/mL IL-2 (PeproTech, Cranbury, NJ), GlutaMAX (Gibco, Waltham, MA), N-acetyl-cysteine (Sigma Aldrich, St. Louis, MO), 5% AB human serum (Valley Biomedical, Winchester, VA), 50 U/mL penicillin and 50 μg/ml streptomycin (Gibco, Waltham, MA). T-cells were stimulated with a 3:1 ratio of Dynabeads™ Human T-Expander CD3/CD28 beads (Thermo Fisher Scientific, Waltham, MA) and cells. At 72 h, the beads were removed and 300,000 T-cells were electroporated with 1 μL (1 μg) of editor mRNA, 1 μL (2 μg) of MLH1dn mRNA, 0.9 μl (100 μM) pegRNA, and 0.6 μl (100 μM) nicking sgRNA using the Neon electroporation system (ThermoFisher) with 10 μl tips and instrument settings of 1,400 V, 10 ms, and 3 pulses. Cells were cultured for 72 hours followed by DNA isolation using the QuickExtract™ DNA Extraction Solution.

### TDT Assay and Analysis

HEK293T cells were transfected in a 96 well plate as described above using 200 ng of editor and 40 ng of pegRNA. (No nicking guides were used for TDT transfections). 24 h after transfection, cells were lysed using 50 μL of lysis buffer per well (47.5 μL Beckman lysis Buffer (Beckman Coulter), 1.25 μL of 1M DTT, and 1.25 μL of proteinase K (Thermo Fisher). Genomic DNA was purified using the Beckman bead purification kit (Beckman Coulter) and eluted in 40 μL of water. 10 μL of purified genomic DNA was used in a 50 μL tailing reaction (1×TDT buffer, 0.25 mM CoCl₂, 100 μM dGTP, 10 units of terminal transferase, NEB). Samples were incubated at 37 °C for 30 min and then 70 °C for 10 min. The tailed DNA was isolated from the reaction mixture using the Beckman bead purification kit again and eluted in 20 μL of water. 5 μL of purified tailed DNA was used as input for a 50 μL PCR1 reaction. TDT PCR1 reactions were performed with Phusion U Green Multiplex PCR Master Mix (25 μL), 5 μL of purified tailed DNA, 19.5 μL of water, and 0.25 μL of 100 μM primers. For TDT assay sequencing, one site-specific primer and one polyC primer were used for PCR1. PCR2 and Miseq were then performed as described above in “HTS sample preparation”.

To analyze TDT samples, a custom Python script provided below was used to analyze demultiplexed fastq files. From top to bottom, the sequences are SEQ ID NOs: 75-78, 111, 112, and 120-132. For scaffold insertion plots (FIG. 11F), TDT results are plotted as the percentage of total edit-containing flaps of a given length. For plots showing the lengths of RTT-encoded flaps synthesized (FIG. 4D and FIG. 11C), all RT products (flaps length 1 or more) were counted, regardless of whether or not they contained the entire edit. Because polyG tailing was used, flap lengths corresponding to a flap ending in G are not detected.

Custom python script used for analyzing TDT sequencing. (Modified from Nelson et
al. 2021)

import pandas as pd
import glob
import re
import os
import subprocess
from subprocess import Popen
from subprocess import PIPE
import Bio as bio
from Bio import SeqIO
from Bio.Seq import Seq
import collections
#if analyzing files where the spacer occurs in the reverse complement of the FASTQ reads,
set #revcomp_mode to True
revcomp_mode = False
fastqs = glob.glob(′*.fastq′)
first10nts = {
′HEK3′: ′GGCCCAGACT′,
′DNMT1′: ′GATTCCTGGT′,
′RNF2′: ′GTCATCTTAG′,
′RUNX1′: ′GCATTTTCAG′,
′VEGFA′: ′GATGTCTGCA′,
′FANCF′: ′GGAATCCCTT′,
′EMX1′: ′GAGTCCGAGC′,
′CCR5′: ′GTATGGAAAA′
}
if revcomp_mode:
for fname in fastqs:
fastqs_rev = list(str(k.seq.reverse_complement()) for k in SeqIO.parse(fname,″fastq″))
with open(f′{fname[:-6]}_trimmed.txt′,′w+′) as f:
for spacer in first10nts.keys():
if spacer in fname:
for entry in fastqs_rev:
nt_read_re = re.search(first1Onts[spacer]+′( .*? )GGGGGGGG′,entry)
try: f.write(str(nt_read_re.group(0))+″\n′)
except: continue
else:
for fname in fastqs:
with open(f′{fname[:-6]}_trimmed.txt′,′w+′) as f:
for spacer in first10nts.keys():
if spacer in fname:
nt_readARGS = [′grep′, ′-o′, f′ {first10nts[spacer]}.*GGGGGGGG′, fname]
nt_readproc = Popen(nt_readARGS, stdout=subprocess.PIPE, universal_newlines = True)
f.write(str(nt_readproc.stdout.read())+″\n′)
#Include reverse complement of spacer in scaffold_revcomp
#If analyzing RT products encoding insertions, set insert_len to length of the insert and
edit_pos to
#where the first mismatched base occurs
#If running in frequency batch mode, set frequency_batch to True
frequency_batch = True
if frequency_batch:
amplicondict = collections.defaultdict(list)
trimmedfastqs = glob.glob(′*trimmed.txt′)
for fname in trimmedfastqs:
sequences_b = open(fname, ′r′)
if ′HEK3′ in fname:
amplicon = ′HEK3′
edit_pos = 1
designed_flap =
′GATTACAAGGATGACGACGATAAGTGATGGCAGAGGAAAGGAAGCCCTGCTT
CCTCCA′
RT_temp_length = 58
insert_len = 24
scaffold_revcomp =
GCACCGACTCGGTGCCACTTTTTCAAGTTGATAACGGACTAGCCTTATTTTAAC
TTGCTATTTCTAGCTCTAAAACTCACGTGCTCAGTCTGGGCC′
if ′RNF2′ in fname:
amplicon = ′RNF2′
edit_pos = 5
designed_flap = ′CTGATGTGTTCGTT′
RT_temp_length = 14
insert_len = 0
scaffold_revcomp =
GCACCGACTCGGTGCCACTTTTTCAAGTTGATAACGGACTAGCCTTATTTTAAC
TTGCTATTTCTAGCTCTAAAACCAGGTAATGACTAAGATGAC′
if ′VEGFA′ in fname:
amplicon = ′VEGFA′
edit_pos = 1
designed_flap =
′GATTACAAGGATGACGACGATAAGTGAGTGCTCCAGATGGCACATT′
RT_temp_length = 46
scaffold_revcomp =
′GCACCGACTCGGTGCCACTTTTTCAAGTTGATAACGGACTAGCCTTATTTTAAC
TTGCTATTTCTAGCTCTAAAACTCATCTGGCCTGCAGACATC′
if ′DNMT1′in fname:
amplicon = ′DNMT1′
edit_pos = 5
designed_flap = ′ACAGTGGTGAC′
RT_temp_length = 11
insert_len = 0
scaffold_revcomp =
′GCACCGACTCGGTGCCACTTTTTCAAGTTGATAACGGACTAGCCTTATTTTAAC
TTGCTATTTCTAGCTCTAAAACGCGCGAACAGCTCCAGCCCGC′
if ′FANCF′ in fname:
amplicon = ′FANCF′
edit_pos = 5
designed_flap = ′ACCTTGATCGCTTTTCC′
RT_temp_length = 17
scaffold_revcomp =
GCACCGACTCGGTGCCACTTTTTCAAGTTGATAACGGACTAGCCTTATTTTAAC
TTGCTATTTCTAGCTCTAAAACGGTGCTGCAGAAGGGATTCC′
if ′EMX1′in fname:
amplicon = ′EMX1′
edit_pos = 5
designed_flap = ′GAAGTGCTCCCATCAC′
RT_temp_length = 16
insert_len = 0
scaffold_revcomp =
′GCACCGACTCGGTGCCACTTTTTCAAGTTGATAACGGACTAGCCTTATTTTAAC
TTGCTATTTCTAGCTCTAAAACTTCTTCTTCTGCTCGGACTC′
seq_list = []
df = pd.DataFrame({′flap seq′: [],′flap length′: [],′contains edit?′: [], ′scaffold
insertion length′: []})
for line in sequences_b:
seq = str(line)
seq_list.append(seq)
counter = 0
counter_b = 1
for read in seq_list:
edit = 0
scaff_RT = 0
for k in range(len(read)-5):
window = read[k:k + 5]
if window == ′GGGGG′:
spacer_flap = read[0:k]
flap_length = len(spacer_flap)-17
if flap_length > edit_pos:
if flap_length < len(designed_flap):
three_prime = flap_length
else:
three_prime = len(designed_flap)
if insert_len > 0:
if len(spacer_flap[17: 17 + three_prime]) > = (insert_len + edit_pos-1):
if spacer_flap[17: 17 + three_prime] == designed_flap[:three_prime]:
edit = 1
elif spacer_flap[17: 17 + three_prime] == designed_flap[:three_prime]:
edit = 1
if flap_length > RT_temp_length:
scaff_ins_len = flap_length-RT_temp_length
scaff_ins_seq = spacer_flap[RT_temp_length + 17:]
if scaffold_revcomp[0:scaff_ins_len] == scaff_ins_seq:
scaff_RT = scaff_ins_len
new_row = pd.DataFrame({′flap seq′: [spacer_flap], ′flap length′: [flap_length], ′contains
edit?′: [edit],′scaffold insertion length′: [scaff_RT]})
df = pd.concat([df,new_row], ignore_index = True)
df.reset_index()
break
df = df[′flap seq′, ′flap length′, ′contains edit?′, ′scaffold insertion length′]]
df.to_csv(f′{fname[:-4]}_output.csv′, index = False)
if frequency_batch:
filedict = {}
flapdict = {}
for i in range(len(df)):
if df.iloc[i,2] == 1.0:
flapdict[df.iloc[i,0] = flapdict.get(df.iloc[i,0],0)+1
flap_denom = sum(flapdict.values())
for k, v in flapdict.items():
flapdict[k] = v/flap_denom
filedict[fname] = flapdict
filedf = pd.DataFrame(filedict)
amplicondict[amplicon].append(filedf)
for amplicon in amplicondict.keys():
amplicondf = pd.concat(amplicondict[amplicon],axis = 1)
amplicondf[′flap length′] = [len(i) for i in amplicondf.index]
amplicondf.sort_values([′flap length′], ascending = True, inplace= True)
amplicondf.to_csv(f′{amplicon}_flapfrequencies.csv′)

### UMI Sample Prep and Analysis

Unique molecular identifiers (UMIs) were applied in a three-step PCR protocol as previously described¹⁰. Briefly, linear amplification was first performed with 1 μL of genomic DNA, Phusion U Green Multiplex PCR Master Mix and 0.1 μM of only the forward primer containing a 15-nt UMI in a 25 μL reaction (eleven cycles of 98 °C for 1 min, 61 °C for 25 s and 72 °C for 1 min). 1.6×AMPure beads (Beckman Coulter) was used to purify the PCR products in 20 μL nuclease-free water, according to the manufacturer's protocol. For the second PCR, a forward primer that binds to the P5 Illumina adaptor sequence located at the 5′ end of the UMI primer was used. This PCR was performed using 2 μL of purified linear DNA, 0.5 μM of each forward and reverse primer and Phusion U Green Multiplex PCR Master Mix for 30 cycles in a 25 μL reaction. In the third PCR, 1 μL of product from the second PCR was amplified for 10 cycles using Phusion U Green Multiplex PCR Master Mix to add unique Illumina barcodes and adaptors as has been described earlier. The products from the third PCR were then pooled, separated by electrophoresis on a 1% agarose gel and purified with QIAquick Gel Extraction Kit (QIAGEN). The library was quantified using Qubit 3.0 Fluorometer (Thermo Fisher Scientific) and finally sequenced using the MiSeq Reagent Kit v2 or MiSeq Reagent Micro Kit v2 (Illumina) with 300 single-read cycles. AmpUMI⁶⁷ was used to UMI deduplicate the raw sequencing reads. The UMI-deduplicated R1s were then analyzed using CRISPResso2 as described earlier⁶⁶.

### AAV Production

AAV production was performed as previously described^{29,74}. HEK293T/17 cells (ATCC) were cultured in DMEM with 10% fetal bovine serum without antibiotics in 150-mm² dishes (Thermo Fisher Scientific) and passaged every 2-3 days at 37 °C with 5% CO₂. Cells were split 1:3, 18-22 hours before transfection. 5.7 μg AAV genome, 11.4 μg pHelper (Clontech), and 22.8 μg AAV9 rep-cap plasmid were transfected per plate using polyethyleneimine (PEI MAX, Polysciences). Media was exchanged for DMEM with 5% fetal bovine serum the following day. Three days after the media change, cells were harvested using a rubber cell scraper (Corning), pelleted via centrifugation (10 min, 2,000 g) and resuspended in 500 μL hypertonic lysis buffer (40 mM Tris base, 2 mM MgCl₂, 500 mM NaCl, and 100 U mL⁻¹ salt active nuclease (ArcticZymes)) per plate, and incubated at 37 °C for 1 hour. The media was decanted and combined with 5× solution of poly(ethylene glycol) (PEG) 8000 (Sigma-Aldrich) and NaCl to achieve a final concentration of 8% PEG and 500 mM NaCl. This solution was incubated on ice for 2 hours or overnight to facilitate PEG precipitation and then centrifuged (3,200 g, 30 minutes). The supernatant was discarded, and the pellet was resuspended in 500 μL hypertonic lysis buffer per plate. This was added to the cell lysate, which was either immediately ultracentrifuged or stored at 4 °C overnight.

Cell lysates were first clarified by centrifugation at 3,400 g for 10 min and added to Beckman Coulter Quick-Seal tubes using a 16-gauge, 5-inch needle (Air-Tite N165) in a discontinuous gradient of iodixanol. The gradient of iodixanol was formed by sequentially floating the following layers: 9 mL 15% iodixanol in 500 mM NaCl and 1×PBS-MK (1×PBS with 2.5 mM KCl, and 1 mM MgCl₂), 6 mL 25% iodixanol in 1×PBS-MK, and 5 mL each of 40% and 60% iodixanol in 1×PBS-MK. Phenol red was added to a final concentration of 1 g mL⁻¹ in the 15, 25, and 60% layers to facilitate layer identification. Ultracentrifugation was performed at 58,600 rpm for 2 hours 15 minutes at 18 °C using a Ti 70 rotor in an Optima XPN-100 Ultracentrifuge (Beckman Coulter). After centrifugation, an 18-gauge needle was used to remove 3 mL of solution from the 40-60% iodixanol interface. This solution was buffer exchanged using PES 100 kD MWCO columns (Thermo Fisher Scientific) with cold PBS containing 0.001% F-68 and finally sterile filtered using a 0.22-μm filter. The final concentrated AAV solution was quantified using qPCR (AAVpro titration kit, Clontech) and stored at 4 °C until use.

### Animals

All mouse experiments were approved by the Broad Institute Institutional Animal Care and Use Committee and consistent with local, state, and federal regulations (as applicable), including the National Institutes of Health Guide for the Care and Use of Laboratory Animals. For P0 studies, timed pregnant C57BL/6J mice were purchased from Charles River Laboratory. All mice were housed in a room maintained on a 12 h light and dark cycle with ad libitum access to standard rodent diet and water.

### P0 Ventricle Injections

P0 ventricle injections were performed as described previously^{29,74}. Drummond PCR pipettes (5-000-1001-X10) were pulled at the ramp test value of a Sutter P1000 micropipette puller and passed through a Kimwipe three times to achieve a tip diameter size of ~100 μm.

To assess ventricle targeting, a small amount of Fast Green dye was added to the AAV injection solution. Using the included Drummond plungers, 4 μL of the injection solution was loaded via front filling. Cryoanestheisa was used to anesthetize the P0 pups. Successful anesthesia was verified by color and unresponsiveness to bilateral toe pinch. Then, 2 μL of the injection solution was injected freehand into each ventricle. Transillumination of the head was used to assess ventricle targeting by the spread of Fast Green throughout the ventricles.

### Mice Tissue Collection

All mice were sacrificed by CO₂ asphyxiation, and tissues were immediately dissected. To harvest the cortex, hemispheres were first split sagittally using a razor blade. The cortex (neocortex+hippocampus) was then isolated using a microspatula.

### Nuclear Isolation and Sorting

Nuclear isolation and sorting were performed as described previously^{29,74}. Dissected cortex tissue was first homogenized using a glass Dounce homogenizer (Sigma-Aldrich; D8938) with 20 strokes of pestle A followed by 20 strokes of pestle B in 2 mL of ice-cold EZ-PREP buffer ((Sigma-Aldrich). Sample was decanted into a new tube with additional 2 mL of cold EZ-PREP buffer on ice and centrifuged (500 g, 4 °C). The supernatant was decanted, and the nuclei pellet was resuspended in 4 mL of ice-cold Nuclei Suspension Buffer (NSB: 100 mg/mL BSA (New England Biolabs) and 3.33 mM Vybrant DyeCycle Ruby (Thermo Fisher) in PBS). The sample was again centrifuged at 500 g for 5 min at 4 °C, the supernatant was decanted, and the nuclei were resuspended in 1 mL of NSB. Samples were passed twice through a 35-μM cell strainer before flow sorting using the Sony MA900 Cell Sorter (Sony Biotechnology) at the Broad Institute flow cytometry core. See FIG. 14B for example FACS gating. Nuclei were sorted into DNAdvance lysis buffer, and the genomic DNA was purified according to the manufacturer's protocol (Beckman Coulter).

### Analysis of Off-Target Editing

Previously identified murine Dnmt1 off-target sites^{30,60} were amplified from either bulk or sorted cells from the mouse cortex. CRISPRESSO was run without an e flag (not in HDR mode), with indels discarded, and with a w value of 20. Off-target edits were counted as leniently as possible: percent off-targets was calculated as the sum of indel reads and editing reads divided by the total number of reads aligned for all amplicons×100. Off-target indels were counted as the number of discarded reads for the sample. To calculate off-target editing events, the pegRNA-encoded sequence was compared to the off-target site. The first SNP at which the two sequences differed was used as a marker for off-target editing: all reads containing that SNP were counted as off-target editing events, even if they did not contain the entire loxP insertion.

### Quantification and Statistical Analysis

The number of independent biological replicates and technical replicates for each experiment are described in the figure legends or the Methods section.

### Example 2. In Vivo Application of PE6 Prime Editors

In Example 1, it was shown that PE6 RT variants improve the installation efficiency of the attB recombinase site into the Rosa26 locus in cultured N2a cells. PE6d yielded an 8.6-fold increase in editing:indel ratio compared to PEmaxΔRNaseH and a 2.4-fold increase in editing:indel ratio compared to PEmax. Next, a series of in vivo prime editing experiments with two PE6 prime editors was completed. The data confirm that PE6 improvements are substantial following in vivo editing in the brain of mice, exceeding an order of magnitude average improvement over previous state-of-the-art PEmaxΔRNaseH.

Neonatal cerebroventricular (P0 ICV) injections of dual AAV delivering PEmaxΔRNaseH or PE6 variants to the CNS of 12 mice were performed (n=4 for each of three groups). Full-length PEmax (together with other required components such as the pegRNA) does not fit into two AAV, necessitating the use of size-reduced prime editors such as PEmaxΔRNaseH or the PE6 variants described herein. An AAV encoding a GFP-KASH fusion was also co-delivered, which enabled isolation of nuclei from AAV-transduced cells via FACS.

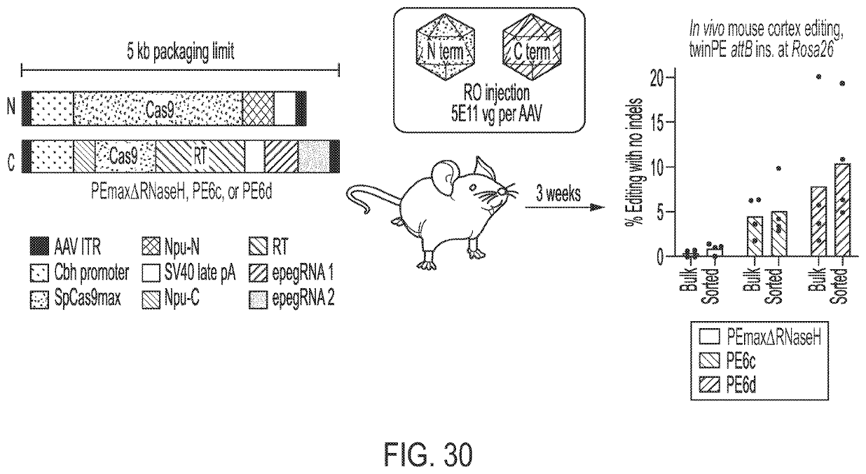

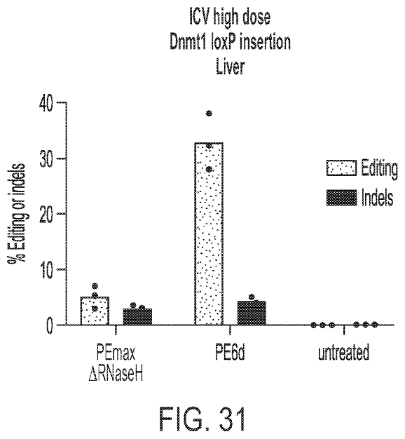

Three weeks after injection, the previous state-of-the-art prime editor PEmaxΔRNaseH yielded low levels of 50-bp attB insertion (0.34% in bulk cortex and 0.9% in GFP+ sorted cells). Both PE6 variants showed substantially higher editing efficiencies (FIG. 30). PE6d yielded 7.8% average prime editing attB installation in bulk brain cortex and 10% in GFP+ sorted cells, with the most efficiently edited mouse reaching 20% of bulk cortex edited. PE6d and PE6c thus increased bulk cortex prime editing efficiency in vivo on average by 23-fold and 13-fold over PEmaxΔRNaseH, respectively.

These data show that PE6's advantages over PEmaxΔRNaseH are critical for in vivo editing. In addition to establishing that the PE6 variants evolved in this study function when administered in vivo, these findings also represent the longest insertion performed using AAV-mediated prime editing in vivo and the first twinPE (dual-flap) edit performed in vivo. The installation of a recombinase site also provides a foundation for in vivo targeted whole-gene insertion, a longstanding goal of the field.

PE6 variants are currently the only prime editors that can efficiently insert long DNA sequences at targeted sites in vivo. In vivo sequence insertion in the brain is an unmet need in the genome editing field, especially since a widely known limitation of homology directed repair (HDR) is its inability to function in post-mitotic cells such as neurons. PE6 variants provide a way to address this longstanding and important challenge.

TABLE 1
Genotypes of RT and Cas9 variants relating to
FIGS. 1A-1J, 3A-3H, 4A-4J, 5A-5H, 6A-6G.
Mutations in RT and Cas9 variants that were tested in
FIGS. 1A-1J, 3A-3H, 4A-4J, 5A-5H, 6A-6G.
FIGS. 1A-1J

| | |
|---|---|
| rdPERV | PERV RT D200N + T306K + W313F + E330P + L603W |
| rdAVIRE | AVIRE RT D200N + T306K + W313F + G330P + L603W |
| rdKORV | KORV RT D198N + W311F + E328P + L600W |
| rdWMSV | WMSV RT D198N + W311F + E328P + L600W |
| rdTf1 | Tf1 RT K118R + S188K + I260L + S297Q + R288Q |
| rdEc48 | Ec48 RT L182N + R315K + T189N |

FIGS. 3A-3H

| | |
|---|---|
| evoGs | Gs RT A16E + L37P + A123V |
| evoEc48 | Ec48 RT E60K + K87E + E165D + D243N + R267I + |
| (PE6a) | E279K + K318E + K343N |
| evoTf1 | Tf1 RT P70T + G72V + S87G + M102I + K106R + |
| (PE6b) | K118R + I128V + L158Q + F269L + A363V + |
| | K413E + S492N |

FIG. 4A-4J

| | |
|---|---|
| PE6c | Tf1 RT P70T + G72V + S87G + M102I + K106R + |
| | K118R + I128V + L158Q + F269L + A363V + K413E + |
| | S492N + K118R + S188K + I260L + S297Q + R288Q |
| PE6d | M-MLV RT T128N + V223Y + D200C + T306K + |
| | W313F (with RNaseH domain truncation of M-MLV |
| | between D497 and I498) |

FIGS. 6A-6G

| | |
|---|---|
| EvoCas9-1 | E102K + R395C + R753G + R778Q + V1100I |
| EvoCas9-2 | R753G + T769P + A1034E + A1320T |
| EvoCas9-3 | D298N + R753G + A1034E + T1138A |
| EvoCas9-4 | R753G + K1151E |
| EvoCas9-5 | E102K + E260K + R395C + R753G + R778Q + |
| | T804A + K1003R + V1100I + S1106F + G1152E |
| EvoCas9-6 | E102K + E260K + R395C + R753G + R778Q + T804A + |
| | K1003R + K1014E + V1100I + S1106F + G1152E |
| PE6e | K918A + K775R |
| PE6f | E471K + H99R + I632V + H721Y + D645N + K918A |
| PE6g | E471K + H99R + I632V + H721Y + R654C + D645N |

TABLE 2
PEgRNA and ngRNA sequences used in Examples:

| | | | | | | | term- |
|---|---|---|---|---|---|---|---|
| | spacer | scaffold | template | PBS | linker | 3′ motif | inator |
| HEK3, +5 G to T | GGCCCAGA | GTTTTAGAGCTAGAAATAGCAAGTT | TCTGCAATCA | CGTGCTCA | | | TTTTTT |
| | CTGAGCAC | AAAATAAGGCTAGTCCGTTATCAAC | | GTCTG | | | |
| | GTGA | TTGAAAAAGTGGGACCGAGTCGGT | | | | | |
| | | CC | | | | | |
| RNF2, +5 G to T | GTCATCTTA | GTTTTAGAGCTAGAAATAGCAAGTT | AACGAACACATCAG | GTAATGAC | | | TTTTTT |
| | GTCATTAC | AAAATAAGGCTAGTCCGTTATCAAC | | TAAGATG | | | |
| | CTG | TTGAAAAAGTGGCACCGAGTCGGT | | | | | |
| | | GC | | | | | |
| HEK3, +1 loxP ins | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCAATAACTTCGTA | | | | |
| | | GC | TAATGTATGCTATAC | | | | |
| | | | GAAGTTATAACAAT | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| HEK3, +1 loxP ins | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCAATAACTTCGTA | | | | |
| | | GC | TAATGTATGCTATAC | | | | |
| | | | GAAGTTATAACAAT | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| CCR5, FKBP ins-twinPE- | GGTACCTA | GTTTTAGAGCTAGAAATAGCAAGTT | ACCTTCCCCAAGCGC | GACAATCG | | | TTTTTT |
| pegRNA1 | TCGATTGTC | AAAATAAGGCTAGTCCGTTATCAAC | GGCCAGACCTGCGTG | AT | | | |
| | AGG | TTGAAAAAGTGGCACCGAGTCGGT | GTGCACTACACCGGG | | | | |
| | | GC | ATGCTTGAAGATGGA | | | | |
| | | | AAGAAATTT | | | | |
| CCR5, FKBP ins-twinPE- | GCTCACTA | GTTTTAGAGCTAGAAATAGCAAGTT | ACCACGCAGGTCTGG | GGCGGCAG | | | TTTTTT |
| pegRNA2 | TGCTGCCG | AAAATAAGGCTAGTCCGTTATCAAC | CCGCGCTTGGGGAAG | CAT | | | |
| | CCCAG | TTGAAAAAGTGGCACCGAGTCGGT | GTGCGCCCGTCTCCT | | | | |
| | | GC | GGGGAGATGGTTTCC | | | | |
| | | | ACCTGCACTCC | | | | |
| IDS edit 1-twinPE-pegRNA1 | GCACTCAT | GTTTTAGAGCTAGAAATAGCAAGTT | GTCTGGTCAACCACC | CTTGGAGG | | | TTTTTT |
| | TTCCTCCAA | AAAATAAGGCTAGTCCGTTATCAAC | GCGGTCTCAGTGGTG | AAA | | | |
| | GCTC | TTGAAAAAGTGGCACCGAGTCGGT | TACGGTACAAACCT | | | | |
| | | GC | | | | | |
| IDS edit 1-twinPE-pegRNA2 | GTTATGGTT | GTTTTAGAGCTAGAAATAGCAAGTT | TACCGTACACCACTG | ATGGAGTA | | | TTTTTT |
| | TACTCCATC | AAAATAAGGCTAGTCCGTTATCAAC | AGACCGCGGTGGTTG | AACC | | | |
| | TA | TTGAAAAAGTGGCACCGAGTCGGT | ACCAGACAAACCT | | | | |
| | | GC | | | | | |
| FANCF, +5 G to T | GGAATCCC | GTTTTAGAGCTAGAAATAGCAAGTT | GGAAAAGCGATCAA | GCTGCAGA | | | TTTTTT |
| | TTCTGCAG | AAAATAAGGCTAGTCCGTTATCAAC | GGT | A | | | |
| | CACC | TTGAAAAAGTGGAGCCGAGTCGGC | | | | | |
| | | TC | | | | | |
| EMX1, +5 G to T | GAGTCCGA | GTTTTAGAGCTAGAAATAGCAAGTT | ATGGGAGCACTTC | TTCTTCTGC | | | TTTTTT |
| | GCAGAAGA | AAAATAAGGCTAGTCCGTTATCAAC | | TCGG | | | |
| | AGAA | TTGAAAAAGTGGCACCGAGTCGGT | | | | | |
| | | GC | | | | | |
| DNMT1, 1-15 deletion | GATTCCTG | GTTTTAGAGCTAGAAATAGCAAGTT | AGGAGGAAGCTGCT | TTCTGGCA | CCTCTTC | TTGACGCGGTTCT | TTTTTT |
| (evopreQ1) | GTGCCAGA | AAAATAAGGCTAGTCCGTTATCAAC | AAGGACTAGTTCTGC | CCAGGA | T | ATCTAGTTACGCG | |
| | AACA | TTGAAAAAGTGGCACCGAGTCGGT | CC | | | TTAAACCAACTAG | |
| | | GC | | | | AAA | |
| HEK3, + 1 FLAG ins | GGCCCAGA | GTTTTAGAGCTAGAAATAGCAAGTT | TGGAGGAAGCAGGG | CGTGCTCA | TCTCTCT | GGGTCAGGAGCCC | TTTTTT |
| (mpknot) | CTGAGCAC | AAAATAAGGCTAGTCCGTTATCAAC | CTTCCTTTCCTCTGCC | GTCTG | C | CCCCCCTGAACCC | |
| | GTGA | TTGAAAAAGTGGCACCGAGTCGGT | ATCACTTATCGTCGT | | | AGGATAACCCTCA | |
| | | GC | CATCCTTGTAATC | | | AAGTCGGGGGGCA | |
| | | | | | | ACCC | |
| RNF2, 1-15 deletion | GTCATCTTA | GTTTTAGAGCTAGAAATAGCAAGTT | TAAGCAAAACATGG | GTAATGAC | TCATCTC | TTGACGCGGTTCT | TTTTTT |
| (evopreQ1) | GTCATTAC | AAAATAAGGCTAGTCCGTTATCAAC | GAACTCAGTTTATAT | TAAGATG | T | ATCTAGTTACGCG | |
| | CTG | TTGAAAAAGTGGCACCGAGTCGGT | GAGTTA | | | TTAAACCAACTAG | |
| | | GC | | | | AAA | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| DNMT1, nicking sgRNA | GCCCTTCA | GTTTTAGAGCTAGAAATAGCAAGTT | | | | | TTTTTT |
| | GCTAAAAT | AAAATAAGGCTAGTCCGTTATCAAC | | | | | |
| | AAAGG | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| EMX1, nicking sgRNA | GCCGTTTGT | GTTTTAGAGCTAGAAATAGCAAGTT | | | | | TTTTTT |
| | ACTTTGTCC | AAAATAAGGCTAGTCCGTTATCAAC | | | | | |
| | TC | TTGAAAAAGTGGCACCGAGTCGGT | | | | | |
| | | GC | | | | | |
| FANCF, nicking sgRNA | GGGGTCCC | GTTTTAGAGCTAGAAATAGCAAGTT | | | | | TTTTTT |
| | AGGTGCTG | AAAATAAGGCTAGTCCGTTATCAAC | | | | | |
| | ACGT | TTGAAAAAGTGGCACCGAGTCGGT | | | | | |
| | | GC | | | | | |
| AAVS1, attP insertion | GACGTCAC | GTTTTAGAGCTAGAAATAGCAAGTT | TACCGTACACCACTG | GGCAGCGC | | | TTTTTT |
| twinPE-pegRNA1 | GGCGCTGC | AAAATAAGGCTAGTCCGTTATCAAC | AGACCGCGGTGGTTG | C | | | |
| | CCCA | TTGAAAAAGTGGCACCGAGTCGGT | ACCAGACAAACCT | | | | |
| | | GC | | | | | |
| AAVS1, attP insertion | GGACTTCC | GTTTTAGAGCTAGAAATAGCAAGTT | GTCTGGTCAACCACC | TGCACACT | | | TTTTTT |
| twinPE-pegRNA2 | CAGTGTGC | AAAATAAGGCTAGTCCGTTATCAAC | GCGGTCTCAGTGGTG | G | | | |
| | ATCG | TTGAAAAAGTGGCACCGAGTCGGT | TACGGTACAAACCT | | | | |
| | | GC | | | | | |
| DNMT1, 1-15 deletion | GATTCCTG | GTTTTAGAGCTAGAAATAGCAAGTT | AGGAGGAAGCTGCT | TTCTGGCA | CCTCTTC | TTGACGCGGTTCT | TTTTTT |
| (evopreQ1) | GTGCCAGA | AAAATAAGGCTAGTCCGTTATCAAC | AAGGACTAGTTCTGC | CCAGGA | T | ATCTAGTTACGCG | |
| | AACA | TTGAAAAAGTGGCACCGAGTCGGT | CC | | | TTAAACCAACTAG | |
| | | GC | | | | AAA | |
| RNF2, 1-15 deletion | GTCATCTTA | GTTTTAGAGCTAGAAATAGCAAGTT | TAAGCAAAACATGG | GTAATGAC | TCATCTC | TTGACGCGGTTCT | TTTTTT |
| (evopreQ1) | GTCATTAC | AAAATAAGGCTAGTCCGTTATCAAC | GAACTCAGTTTATAT | TAAGATG | T | ATCTAGTTACGCG | |
| | CTG | TTGAAAAAGTGGCACCGAGTCGGT | GAGTTA | | | TTAAACCAACTAG | |
| | | GC | | | | AAA | |
| EMX1, +5 G to T | GAGTCCGA | GTTTTAGAGCTAGAAATAGCAAGTT | ATGGGAGCACTTC | TTCTTCTGC | | | TTTTTT |
| | GCAGAAGA | AAAATAAGGCTAGTCCGTTATCAAC | | TCGG | | | |
| | AGAA | TTGAAAAAGTGGCACCGAGTCGGT | | | | | |
| | | GC | | | | | |
| FANCF, +5 G to T | GGAATCCC | GTTTTAGAGCTAGAAATAGCAAGTT | GGAAAAGCGATCAA | GCTGCAGA | | | TTTTTT |
| | TTCTGCAG | AAAATAAGGCTAGTCCGTTATCAAC | GGT | A | | | |
| | CACC | TTGAAAAAGTGGCACCGAGTCGGT | | | | | |
| | | GC | | | | | |
| DNMT1, nicking sgRNA | GCCCTTCA | GTTTTAGAGCTAGAAATAGCAAGTT | | | | | TTTTTT |
| | GCTAAAAT | AAAATAAGGCTAGTCCGTTATCAAC | | | | | |
| | AAAGG | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| EMX1, nicking sgRNA | GCCGTTTGT | GTTTTAGAGCTAGAAATAGCAAGTT | | | | | TTTTTT |
| | ACTTTGTCC | AAAATAAGGCTAGTCCGTTATCAAC | | | | | |
| | TC | TTGAAAAAGTGGCACCGAGTCGGT | | | | | |
| | | GC | | | | | |
| FANCF, nicking sgRNA | GGGGTCCC | GTTTTAGAGCTAGAAATAGCAAGTT | | | | | TTTTTT |
| | AGGTGCTG | AAAATAAGGCTAGTCCGTTATCAAC | | | | | |
| | ACGT | TTGAAAAAGTGGCACCGAGTCGGT | | | | | |
| | | GC | | | | | |
| DNMT1, 1-15 deletion | GATTCCTG | GTTTTAGAGCTAGAAATAGCAAGTT | AGGAGGAAGCTGCT | TTCTGGCA | CCTCTTC | TTGACGCGGTTCT | TTTTTT |
| (evopreQ1) | GTGCCAGA | AAAATAAGGCTAGTCCGTTATCAAC | AAGGACTAGTTCTGC | CCAGGA | T | ATCTAGTTACGCG | |
| | AACA | TTGAAAAAGTGGCACCGAGTCGGT | CC | | | TTAAACCAACTAG | |
| | | GC | | | | AAA | |
| HEK3, + 1 FLAG ins | GGCCCAGA | GTTTTAGAGCTAGAAATAGCAAGTT | TGGAGGAAGCAGGG | CGTGCTCA | TCTCTCT | GGGTCAGGAGCCC | TTTTTT |
| (mpknot) | CTGAGCAC | AAAATAAGGCTAGTCCGTTATCAAC | CTTCCTTTCCTCTGCC | GTCTG | C | CCCCCCTGAACCC | |
| | GTGA | TTGAAAAAGTGGCACCGAGTCGGT | ATCACTTATCGTCGT | | | AGGATAACCCTCA | |
| | | GC | CATCCTTGTAATC | | | AAGTCGGGGGGCA | |
| | | | | | | ACCC | |
| RNF2, 1-15 deletion | GTCATCTTA | GTTTTAGAGCTAGAAATAGCAAGTT | TAAGCAAAACATGG | GTAATGAC | TCATCTC | TTGACGCGGTTCT | TTTTTT |
| (evopreQ1) | GTCATTAC | AAAATAAGGCTAGTCCGTTATCAAC | GAACTCAGTTTATAT | TAAGATG | T | ATCTAGTTACGCG | |
| | CTG | TTGAAAAAGTGGCACCGAGTCGGT | GAGTTA | | | TTAAACCAACTAG | |
| | | GC | | | | AAA | |
| EMX1, +5 G to T | GAGTCCGA | GTTTTAGAGCTAGAAATAGCAAGTT | ATGGGAGCACTTC | TTCTTCTGC | | | TTTTTT |
| | GCAGAAGA | AAAATAAGGCTAGTCCGTTATCAAC | | TCGG | | | |
| | AGAA | TTGAAAAAGTGGCACCGAGTCGGT | | | | | |
| | | GC | | | | | |
| FANCF, +5 G to T | GGAATCCC | GTTTTAGAGCTAGAAATAGCAAGTT | GGAAAAGCGATCAA | GCTGCAGA | | | TTTTTT |
| | TTCTGCAG | AAAATAAGGCTAGTCCGTTATCAAC | GGT | A | | | |
| | CACC | TTGAAAAAGTGGCACCGAGTCGGT | | | | | |
| | | GC | | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| DNMT1, nicking sgRNA | GCCCTTCA | GTTTTAGAGCTAGAAATAGCAAGTT | | | | | TTTTTT |
| | GCTAAAAT | AAAATAAGGCTAGTCCGTTATCAAC | | | | | |
| | AAAGG | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| EMX1, nicking sgRNA | GCCGTTTGT | GTTTTAGAGCTAGAAATAGCAAGTT | | | | | TTTTTT |
| | ACTTTGTCC | AAAATAAGGCTAGTCCGTTATCAAC | | | | | |
| | TC | TTGAAAAAGTGGCACCGAGTCGGT | | | | | |
| | | GC | | | | | |
| FANCF, nicking sgRNA | GGGGTCCC | GTTTTAGAGCTAGAAATAGCAAGTT | | | | | TTTTTT |
| | AGGTGCTG | AAAATAAGGCTAGTCCGTTATCAAC | | | | | |
| | ACGT | TTGAAAAAGTGGCACCGAGTCGGT | | | | | |
| | | GC | | | | | |
| AAVS1, attP insertion | GACGTCAC | GTTTTAGAGCTAGAAATAGCAAGTT | TACCGTACACCACTG | GGCAGCGC | | | TTTTTT |
| twinPE-pegRNA1 | GGCGCTGC | AAAATAAGGCTAGTCCGTTATCAAC | AGACCGCGGTGGTTG | C | | | |
| | CCCA | TTGAAAAAGTGGCACCGAGTCGGT | ACCAGACAAACCT | | | | |
| | | GC | | | | | |
| AAVS1, attP insertion | GGACTTCC | GTTTTAGAGCTAGAAATAGCAAGTT | GTCTGGTCAACCACC | TGCACACT | | | TTTTTT |
| twinPE-pegRNA2 | CAGTGTGC | AAAATAAGGCTAGTCCGTTATCAAC | GCGGTCTCAGTGGTG | G | | | |
| | ATCG | TTGAAAAAGTGGCACCGAGTCGGT | TACGGTACAAACCT | | | | |
| | | GC | | | | | |
| DNMT1, 1-15 deletion | GATTCCTG | GTTTTAGAGCTAGAAATAGCAAGTT | AGGAGGAAGCTGCT | TTCTGGCA | CCTCTTC | TTGACGCGGTTCT | TTTTTT |
| (evopreQ1) | GTGCCAGA | AAAATAAGGCTAGTCCGTTATCAAC | AAGGACTAGTTCTGC | CCAGGA | T | ATCTAGTTACGCG | |
| | AACA | TTGAAAAAGTGGCACCGAGTCGGT | CC | | | TTAAACCAACTAG | |
| | | GC | | | | AAA | |
| HEK3, + 1 FLAG ins | GGCCCAGA | GTTTTAGAGCTAGAAATAGCAAGTT | TGGAGGAAGCAGGG | CGTGCTCA | TCTCTCT | GGGTCAGGAGCCC | TTTTTT |
| (mpknot) | CTGAGCAC | AAAATAAGGCTAGTCCGTTATCAAC | CTTCCTTTCCTCTGCC | GTCTG | C | CCCCCCTGAACCC | |
| | GTGA | TTGAAAAAGTGGCACCGAGTCGGT | ATCACTTATCGTCGT | | | AGGATAACCCTCA | |
| | | GC | CATCCTTGTAATC | | | AAGTCGGGGGGCA | |
| | | | | | | ACCC | |
| RNF2, 1-15 deletion | GTCATCTTA | GTTTTAGAGCTAGAAATAGCAAGTT | TAAGCAAAACATGG | GTAATGAC | TCATCTC | TTGACGCGGTTCT | TTTTTT |
| (evopreQ1) | GTCATTAC | AAAATAAGGCTAGTCCGTTATCAAC | GAACTCAGTTTATAT | TAAGATG | T | ATCTAGTTACGCG | |
| | CTG | TTGAAAAAGTGGCACCGAGTCGGT | GAGTTA | | | TTAAACCAACTAG | |
| | | GC | | | | AAA | |
| EMX1, +5 G to T | GAGTCCGA | GTTTTAGAGCTAGAAATAGCAAGTT | ATGGGAGCACTTC | TTCTTCTGC | | | TTTTTT |
| | GCAGAAGA | AAAATAAGGCTAGTCCGTTATCAAC | | TCGG | | | |
| | AGAA | TTGAAAAAGTGGCACCGAGTCGGT | | | | | |
| | | GC | | | | | |
| FANCF, +5 G to T | GGAATCCC | GTTTTAGAGCTAGAAATAGCAAGTT | GGAAAAGCGATCAA | GCTGCAGA | | | TTTTTT |
| | TTCTGCAG | AAAATAAGGCTAGTCCGTTATCAAC | GGT | A | | | |
| | CACC | TTGAAAAAGTGGCACCGAGTCGGT | | | | | |
| | | GC | | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| DNMT1, nicking sgRNA | GCCCTTCA | GTTTTAGAGCTAGAAATAGCAAGTT | | | | | TTTTTT |
| | GCTAAAAT | AAAATAAGGCTAGTCCGTTATCAAC | | | | | |
| | AAAGG | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| EMX1, nicking sgRNA | GCCGTTTGT | GTTTTAGAGCTAGAAATAGCAAGTT | | | | | TTTTTT |
| | ACTTTGTCC | AAAATAAGGCTAGTCCGTTATCAAC | | | | | |
| | TC | TTGAAAAAGTGGCACCGAGTCGGT | | | | | |
| | | GC | | | | | |
| FANCF, nicking sgRNA | GGGGTCCC | GTTTTAGAGCTAGAAATAGCAAGTT | | | | | TTTTTT |
| | AGGTGCTG | AAAATAAGGCTAGTCCGTTATCAAC | | | | | |
| | ACGT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | AGACTCATGCTCAAG | GCCAACTG | | | TTTTTT |
| T7 RNAP, (+6 ins A, v1 | ACAGTTGG | AAAATAAGGCTAGTCCGTTATCAAC | G | TTCGC | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | ACTCATGCTCAAGG | GCCAACTG | | | TTTTTT |
| T7 RNAP, (+6 ins A, v1 | ACAGTTGG | AAAATAAGGCTAGTCCGTTATCAAC | | T | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | AGACTCATGCTCAAG | GCCAACTG | | | TTTTTT |
| T7 RNAP, (+6 ins A, v1 | ACAGTTGG | AAAATAAGGCTAGTCCGTTATCAAC | G | T | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | GTAAGACTCATGCTC | GCCAACTG | | | TTTTTT |
| T7 RNAP, (+6 ins A, v1 | ACAGTTGG | AAAATAAGGCTAGTCCGTTATCAAC | AAGG | T | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | TCGTAAGACTCATGC | GCCAACTG | | | TTTTTT |
| T7 RNAP, (+6 ins A, v1 | ACAGTTGG | AAAATAAGGCTAGTCCGTTATCAAC | TCAAGG | T | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | ATCTCGTAAGACTCA | GCCAACTG | | | TTTTTT |
| T7 RNAP, (+6 ins A, v1 | ACAGTTGG | AAAATAAGGCTAGTCCGTTATCAAC | TGCTCAAGG | T | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | TTCACCCATCTCGTA | GCCAACTG | | | TTTTTT |
| T7 RNAP, (+6 ins A, v1 | ACAGTTGG | AAAATAAGGCTAGTCCGTTATCAAC | AGACTCATGCTCAAG | T | | | |
| circuit) | CCCT | TTGAAAAAGTGGCACCGAGTCGGT | G | | | | |
| | | GC | | | | | |
| Correction of 1-bp deletion in | GCTCGCGA | GTTTTAGAGCTAGAAATAGCAAGTT | ACTCATGCTCAAGG | GCCAACTG | | | TTTTTT |
| T7 RNAP, (+6 ins A, v1 | ACAGTTGG | AAAATAAGGCTAGTCCGTTATCAAC | | TT | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | AGACTCATGCTCAAG | GCCAACTG | | | TTTTTT |
| T7 RNAP, (+6 ins A, v1 | ACAGTTGG | AAAATAAGGCTAGTCCGTTATCAAC | G | TT | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | GTAAGACTCATGCTC | GCCAACTG | | | TTTTTT |
| T7 RNAP, (+6 ins A, v1 | ACAGTTGG | AAAATAAGGCTAGTCCGTTATCAAC | AAGG | TT | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | TCGTAAGACTCATGC | GCCAACTG | | | TTTTTT |
| T7 RNAP, (+6 ins A, v1 | ACAGTTGG | AAAATAAGGCTAGTCCGTTATCAAC | TCAAGG | TT | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | ATCTCGTAAGACTCA | GCCAACTG | | | TTTTTT |
| T7 RNAP, (+6 ins A, v1 | ACAGTTGG | AAAATAAGGCTAGTCCGTTATCAAC | TGCTCAAGG | TT | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | TTCACCCATCTCGTA | GCCAACTG | | | TTTTTT |
| T7 RNAP, (+6 ins A, v1 | ACAGTTGG | AAAATAAGGCTAGTCCGTTATCAAC | AGACTCATGCTCAAG | TT | | | |
| circuit) | CCCT | TTGAAAAAGTGGCACCGAGTCGGT | G | | | | |
| | | GC | | | | | |
| Correction of 1-bp deletion in | GCTCGCGA | GTTTTAGAGCTAGAAATAGCAAGTT | ACTCATGCTCAAGG | GCCAACTG | | | TTTTTT |
| T7 RNAP, (+6 ins A, v1 | ACAGTTGG | AAAATAAGGCTAGTCCGTTATCAAC | | TTC | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | AGACTCATGCTCAAG | GCCAACTG | | | TTTTTT |
| T7 RNAP, (+6 ins A, v1 | ACAGTTGG | AAAATAAGGCTAGTCCGTTATCAAC | G | TTC | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | GTAAGACTCATGCTC | GCCAACTG | | | TTTTTT |
| T7 RNAP, (+6 ins A, v1 | ACAGTTGG | AAAATAAGGCTAGTCCGTTATCAAC | AAGG | TTC | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | TCGTAAGACTCATGC | GCCAACTG | | | TTTTTT |
| T7 RNAP, (+6 ins A, v1 | ACAGTTGG | AAAATAAGGCTAGTCCGTTATCAAC | TCAAGG | TTC | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | ATCTCGTAAGACTCA | GCCAACTG | | | TTTTTT |
| T7 RNAP, (+6 ins A, v1 | ACAGTTGG | AAAATAAGGCTAGTCCGTTATCAAC | TGCTCAAGG | TTC | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | TTCACCCATCTCGTA | GCCAACTG | | | TTTTTT |
| T7 RNAP, (+6 ins A, v1 | ACAGTTGG | AAAATAAGGCTAGTCCGTTATCAAC | AGACTCATGCTCAAG | TTC | | | |
| circuit) | CCCT | TTGAAAAAGTGGCACCGAGTCGGT | G | | | | |
| | | GC | | | | | |
| Correction of 1-bp deletion in | GCTCGCGA | GTTTTAGAGCTAGAAATAGCAAGTT | ACTCATGCTCAAGG | GCCAACTG | | | TTTTTT |
| T7 RNAP, (+6 ins A, v1 | ACAGTTGG | AAAATAAGGCTAGTCCGTTATCAAC | | TTCG | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | AGACTCATGCTCAAG | GCCAACTG | | | TTTTTT |
| T7 RNAP, (+6 ins A, v1 | ACAGTTGG | AAAATAAGGCTAGTCCGTTATCAAC | G | TTCG | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | GTAAGACTCATGCTC | GCCAACTG | | | TTTTTT |
| T7 RNAP, (+6 ins A, v1 | ACAGTTGG | AAAATAAGGCTAGTCCGTTATCAAC | AAGG | TTCG | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | TCGTAAGACTCATGC | GCCAACTG | | | TTTTTT |
| T7 RNAP, (+6 ins A, v1 | ACAGTTGG | AAAATAAGGCTAGTCCGTTATCAAC | TCAAGG | TTCG | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | ATCTCGTAAGACTCA | GCCAACTG | | | TTTTTT |
| T7 RNAP, (+6 ins A, v1 | ACAGTTGG | AAAATAAGGCTAGTCCGTTATCAAC | TGCTCAAGG | TTCG | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | TTCACCCATCTCGTA | GCCAACTG | | | TTTTTT |
| T7 RNAP, (+6 ins A, v1 | ACAGTTGG | AAAATAAGGCTAGTCCGTTATCAAC | AGACTCATGCTCAAG | TTCG | | | |
| circuit) | CCCT | TTGAAAAAGTGGCACCGAGTCGGT | G | | | | |
| | | GC | | | | | |
| Correction of 1-bp deletion in | GCTCGCGA | GTTTTAGAGCTAGAAATAGCAAGTT | ACTCATGCTCAAGG | GCCAACTG | | | TTTTTT |
| T7 RNAP, (+6 ins A, v1 | ACAGTTGG | AAAATAAGGCTAGTCCGTTATCAAC | | TTCGC | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | AGACTCATGCTCAAG | GCCAACTG | | | TTTTTT |
| T7 RNAP, (+6 ins A, v1 | ACAGTTGG | AAAATAAGGCTAGTCCGTTATCAAC | G | TTCGC | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | GTAAGACTCATGCTC | GCCAACTG | | | TTTTTT |
| T7 RNAP, (+6 ins A, v1 | ACAGTTGG | AAAATAAGGCTAGTCCGTTATCAAC | AAGG | TTCGC | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | TCGTAAGACTCATGC | GCCAACTG | | | TTTTTT |
| T7 RNAP, (+6 ins A, v1 | ACAGTTGG | AAAATAAGGCTAGTCCGTTATCAAC | TCAAGG | TTCGC | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | TTCACCCATCTCGTA | GCCAACTG | | | TTTTTT |
| T7 RNAP, (+6 ins A, v1 | ACAGTTGG | AAAATAAGGCTAGTCCGTTATCAAC | AGACTCATGCTCAAG | TTCGC | | | |
| circuit) | CCCT | TTGAAAAAGTGGCACCGAGTCGGT | G | | | | |
| | | GC | | | | | |
| Correction of 1-bp deletion in | GCTCGCGA | GTTTTAGAGCTAGAAATAGCAAGTT | ACTCATGCTCAAGG | GCCAACTG | | | TTTTTT |
| T7 RNAP, (+6 ins A, v1 | ACAGTTGG | AAAATAAGGCTAGTCCGTTATCAAC | | TTCGCG | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | AGACTCATGCTCAAG | GCCAACTG | | | TTTTTT |
| T7 RNAP, (+6 ins A, v1 | ACAGTTGG | AAAATAAGGCTAGTCCGTTATCAAC | G | TTCGCG | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | GTAAGACTCATGCTC | GCCAACTG | | | TTTTTT |
| T7 RNAP, (+6 ins A, v1 | ACAGTTGG | AAAATAAGGCTAGTCCGTTATCAAC | AAGG | TTCGCG | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | TCGTAAGACTCATGC | GCCAACTG | | | TTTTTT |
| T7 RNAP, (+6 ins A, v1 | ACAGTTGG | AAAATAAGGCTAGTCCGTTATCAAC | TCAAGG | TTCGCG | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | ATCTCGTAAGACTCA | GCCAACTG | | | TTTTTT |
| T7 RNAP, (+6 ins A, v1 | ACAGTTGG | AAAATAAGGCTAGTCCGTTATCAAC | TGCTCAAGG | TTCGCG | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | TTCACCCATCTCGTA | GCCAACTG | | | TTTTTT |
| T7 RNAP, (+6 ins A, v1 | ACAGTTGG | AAAATAAGGCTAGTCCGTTATCAAC | AGACTCATGCTCAAG | TTCGCG | | | |
| circuit) | CCCT | TTGAAAAAGTGGCACCGAGTCGGT | G | | | | |
| | | GC | | | | | |
| Correction of 1-bp deletion in | GCTCGCGA | GTTTTAGAGCTAGAAATAGCAAGTT | GTAAGACTCATGCTC | GCCAACTG | | | TTTTTT |
| T7 RNAP, (+6 ins A, v1 | ACAGTTGG | AAAATAAGGCTAGTCCGTTATCAAC | AAGG | TT | | | |
| circuit) | CCCT | TTGAAAAAGTGGCACCGAGTCGGT | | | | | |
| | | GC | | | | | |
| Correction of 1-bp deletion in | GCTCGCGA | GTTTTAGAGCTAGAAATAGCAAGTT | AGACTCATGCTCAAG | GCCAACTG | | | TTTTTT |
| T7 RNAP, (+6 ins A, v1 | ACAGTTGG | AAAATAAGGCTAGTCCGTTATCAAC | G | T | | | |
| circuit) | CCCT | TTGAAAAAGTGGCACCGAGTCGGT | | | | | |
| | | GC | | | | | |
| Correction of 20-bp deletion | GCAATGAA | GTTTTAGAGCTAGAAATAGCAAGTT | TTGCGAAGAATCTCT | TTAAGCCA | | | TTTTTT |
| in T7 RNAP, (v2 circuit) | CTGGCTTA | AAAATAAGGCTAGTCCGTTATCAAC | CCAGTCTTCTTATCTT | G | | | |
| | AGTC | TTGAAAAAGTGGCACCGAGTCGGT | TGACCTCAGCAGCCA | | | | |
| | | GC | GCAGCTTAGCAGCAG | | | | |
| | | | AC | | | | |
| AAVS1, attP insertion | GACGTCAC | GTTTTAGAGCTAGAAATAGCAAGTT | TACCGTACACCACTG | GGCAGCGC | | | TTTTTT |
| twinPE-pegRNA1 | GGCGCTGC | AAAATAAGGCTAGTCCGTTATCAAC | AGACCGCGGTGGTTG | C | | | |
| | CCCA | TTGAAAAAGTGGCACCGAGTCGGT | ACCAGACAAACCT | | | | |
| | | GC | | | | | |
| AAVS1, attP insertion | GGACTTCC | GTTTTAGAGCTAGAAATAGCAAGTT | GTCTGGTCAACCACC | TGCACACT | | | TTTTTT |
| twinPE-pegRNA2 | CAGTGTGC | AAAATAAGGCTAGTCCGTTATCAAC | GCGGTCTCAGTGGTG | G | | | |
| | ATCG | TTGAAAAAGTGGCACCGAGTCGGT | TACGGTACAAACCT | | | | |
| | | GC | | | | | |
| FANCF, +5 G to T (no | GGAATCCC | GTTTTAGAGCTAGAAATAGCAAGTT | GGAAAAGCGATCAA | GCTGCAGA | | | TTTTTT |
| ngRNA) | TTCTGCAG | AAAATAAGGCTAGTCCGTTATCAAC | GGT | A | | | |
| | CACC | TTGAAAAAGTGGCACCGAGTCGGT | | | | | |
| | | GC | | | | | |
| EMX1, +5 G to T | GAGTCCGA | GTTTTAGAGCTAGAAATAGCAAGTT | ATGGGAGCACTTC | TTCTTCTGC | | | TTTTTT |
| | GCAGAAGA | AAAATAAGGCTAGTCCGTTATCAAC | | TCGG | | | |
| | AGAA | TTGAAAAAGTGGCACCGAGTCGGT | | | | | |
| | | GC | | | | | |
| DNMT1, 1-15 deletion | GATTCCTG | GTTTTAGAGCTAGAAATAGCAAGTT | AGGAGGAAGCTGCT | TTCTGGCA | CCTCTTC | TTGACGCGGTTCT | TTTTTT |
| (evopreQ1, no ngRNA) | GTGCCAGA | AAAATAAGGCTAGTCCGTTATCAAC | AAGGACTAGTTCTGC | CCAGGA | T | ATCTAGTTACGCG | |
| | AACA | TTGAAAAAGTGGCACCGAGTCGGT | CC | | | TTAAACCAACTAG | |
| | | GC | | | | AAA | |
| HEK3, + 1 FLAG ins | GGCCCAGA | GTTTTAGAGCTAGAAATAGCAAGTT | TGGAGGAAGCAGGG | CGTGCTCA | TCTCTCT | GGGTCAGGAGCCC | TTTTTT |
| (mpknot) | CTGAGCAC | AAAATAAGGCTAGTCCGTTATCAAC | CTTCCTTTCCTCTGCC | GTCTG | C | CCCCCCTGAACCC | |
| | GTGA | TTGAAAAAGTGGCACCGAGTCGGT | ATCACTTATCGTCGT | | | AGGATAACCCTCA | |
| | | GC | CATCCTTGTAATC | | | AAGTCGGGGGGCA | |
| | | | | | | ACCC | |
| RNF2, 1-15 deletion | GTCATCTTA | GTTTTAGAGCTAGAAATAGCAAGTT | TAAGCAAAACATGG | GTAATGAC | TCATCTC | TTGACGCGGTTCT | TTTTTT |
| (evopreQ1) | GTCATTAC | AAAATAAGGCTAGTCCGTTATCAAC | GAACTCAGTTTATAT | TAAGATG | T | ATCTAGTTACGCG | |
| | CTG | TTGAAAAAGTGGCACCGAGTCGGT | GAGTTA | | | TTAAACCAACTAG | |
| | | GC | | | | AAA | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| EMX1, nicking sgRNA | GCCGTTTGT | GTTTTAGAGCTAGAAATAGCAAGTT | | | | | TTTTTT |
| | ACTTTGTCC | AAAATAAGGCTAGTCCGTTATCAAC | | | | | |
| | TC | TTGAAAAAGTGGCACCGAGTCGGT | | | | | |
| | | GC | | | | | |
| VEGFA, +5 G to T | GATGTCTG | GTTTTAGAGCTAGAAATAGCAAGTT | AATGTGCCATCTGGA | TCTGGCCT | ACAATCT | TTGACGCGGTTCT | TTTTTT |
| (evopreQ1) | CAGGCCAG | AAAATAAGGCTAGTCCGTTATCAAC | GCACTCA | GCAGA | C | ATCTAGTTACGCG | |
| | ATGA | TTGAAAAAGTGGCACCGAGTCGGT | | | | TTAAACCAACTAG | |
| | | GC | | | | AAA | |
| DNMT1, 1-15 deletion | GATTCCTG | GTTTTAGAGCTAGAAATAGCAAGTT | AGGAGGAAGCTGCT | TTCTGGCA | CCTCTTC | TTGACGCGGTTCT | TTTTTT |
| (evopreQ1) | GTGCCAGA | AAAATAAGGCTAGTCCGTTATCAAC | AAGGACTAGTTCTGC | CCAGGA | T | ATCTAGTTACGCG | |
| | AACA | TTGAAAAAGTGGCACCGAGTCGGT | CC | | | TTAAACCAACTAG | |
| | | GC | | | | AAA | |
| RNF2, 1-15 deletion | GTCATCTTA | GTTTTAGAGCTAGAAATAGCAAGTT | TAAGCAAAACATGG | GTAATGAC | TCATCTC | TTGACGCGGTTCT | TTTTTT |
| (evopreQ1) | GTCATTAC | AAAATAAGGCTAGTCCGTTATCAAC | GAACTCAGTTTATAT | TAAGATG | T | ATCTAGTTACGCG | |
| | CTG | TTGAAAAAGTGGCACCGAGTCGGT | GAGTTA | | | TTAAACCAACTAG | |
| | | GC | | | | AAA | |
| RUNX1, +5 G to T | GCATTTTCA | GTTTTAGAGCTAGAAATAGCAAGTT | AGGAAATGACTCAA | CTTCCTCCT | AACTCTC | TTGACGCGGTTCT | TTTTTT |
| (evopreQ1) | GGAGGAAG | AAAATAAGGCTAGTCCGTTATCAAC | ATATGCTGTCTGAAG | GAAAAT | T | ATCTAGTTACGCG | |
| | CGA | TTGAAAAAGTGGCACCGAGTCGGT | CAATCG | | | TTAAACCAACTAG | |
| | | GC | | | | AAA | |
| EMX1, 1-15 deletion | GAGTCCGA | GTTTTAGAGCTAGAAATAGCAAGTT | TTGGCCTGCTTCGTG | TTCTTCTGC | AACAATC | TTGACGCGGTTCT | TTTTTT |
| (evopreQ1) | GCAGAAGA | AAAATAAGGCTAGTCCGTTATCAAC | GCAATGCGCCACCGG | TCGGA | T | ATCTAGTTACGCG | |
| | AGAA | TTGAAAAAGTGGCACCGAGTCGGT | TTGATG | | | TTAAACCAACTAG | |
| | | GC | | | | AAA | |
| FANCF, +5 G to T (mpknot) | GGAATCCC | GTTTTAGAGCTAGAAATAGCAAGTT | GGAAAAGCGATCAA | GCTGCAGA | CAATCAC | GGGTCAGGAGCCC | TTTTTT |
| | TTCTGCAG | AAAATAAGGCTAGTCCGTTATCAAC | GGT | AGGGA | T | CCCCCCTGAACCC | |
| | CACC | TTGAAAAAGTGGCACCGAGTCGGT | | | | AGGATAACCCTCA | |
| | | GC | | | | AAGTCGGGGGGCA | |
| | | | | | | ACCC | |
| EMX1, +5 G to T (evopreQ1) | GAGTCCGA | GTTTTAGAGCTAGAAATAGCAAGTT | GTGATGGGAGCACTT | TTCTTCTGC | AACAATC | TTGACGCGGTTCT | TTTTTT |
| | GCAGAAGA | AAAATAAGGCTAGTCCGTTATCAAC | C | TCGGA | T | ATCTAGTTACGCG | |
| | AGAA | TTGAAAAAGTGGCACCGAGTCGGT | | | | TTAAACCAACTAG | |
| | | GC | | | | AAA | |
| VEGFA, nicking sgRNA | GAGCCCAG | GTTTTAGAGCTAGAAATAGCAAGTT | | | | | TTTTTT |
| | GGCTGGGC | AAAATAAGGCTAGTCCGTTATCAAC | | | | | |
| | ACAG | TTGAAAAAGTGGCACCGAGTCGGT | | | | | |
| | | GC | | | | | |
| RUNX1, nicking sgRNA | GATGAAGC | GTTTTAGAGCTAGAAATAGCAAGTT | | | | | TTTTTT |
| | ACTGTGGG | AAAATAAGGCTAGTCCGTTATCAAC | | | | | |
| | TACGA | TTGAAAAAGTGGCACCGAGTCGGT | | | | | |
| | | GC | | | | | |
| DNMT1, nicking sgRNA | GCCCTTCA | GTTTTAGAGCTAGAAATAGCAAGTT | | | | | TTTTTT |
| | GCTAAAAT | AAAATAAGGCTAGTCCGTTATCAAC | | | | | |
| | AAAGG | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| EMX1, nicking sgRNA | GCCGTTTGT | GTTTTAGAGCTAGAAATAGCAAGTT | | | | | TTTTTT |
| | ACTTTGTCC | AAAATAAGGCTAGTCCGTTATCAAC | | | | | |
| | TC | TTGAAAAAGTGGCACCGAGTCGGT | | | | | |
| | | GC | | | | | |
| FANCF, nicking sgRNA | GGGGTCCC | GTTTTAGAGCTAGAAATAGCAAGTT | | | | | TTTTTT |
| | AGGTGCTG | AAAATAAGGCTAGTCCGTTATCAAC | | | | | |
| | ACGT | TTGAAAAAGTGGCACCGAGTCGGT | | | | | |
| | | GC | | | | | |
| HEXA 1278insTATC | GATCCTTCC | GTTTTAGAGCTAGAAATAGCAAGTT | GTACCTGAACCGTAT | GCCCTGAC | TCTCTCT | CGCGGTTCTATCTA | TTTTTT |
| | AGTCAGGG | AAAATAAGGCTAGTCCGTTATCAAC | ATCGTATG | T | C | GTTACGCGTTAAA | |
| | CCAT | TTGAAAAAGTGGCACCGAGTCGGT | | | | CCAACTAGAA | |
| | | GC | | | | | |
| HEXA, nicking sgRNA | GTACCTGA | GTTTTAGAGCTAGAAATAGCAAGTT | | | | | TTTTTT |
| | ACCGTATA | AAAATAAGGCTAGTCCGTTATCAAC | | | | | |
| | TCGTA | TTGAAAAAGTGGCACCGAGTCGGT | | | | | |
| | | GC | | | | | |
| HEK3, + 1 FLAG ins | GGCCCAGA | GTTTTAGAGCTAGAAATAGCAAGTT | TGGAGGAAGCAGGG | CGTGCTCA | TCTCTCT | GGGTCAGGAGCCC | TTTTTT |
| (mpknot) | CTGAGCAC | AAAATAAGGCTAGTCCGTTATCAAC | CTTCCTTTCCTCTGCC | GTCTG | C | CCCCCCTGAACCC | |
| | GTGA | TTGAAAAAGTGGCACCGAGTCGGT | ATCACTTATCGTCGT | | | AGGATAACCCTCA | |
| | | GC | CATCCTTGTAATC | | | AAGTCGGGGGGCA | |
| | | | | | | ACCC | |
| HEK3, +1 loxP ins | GGCCCAGA | GTTTTAGAGCTAGAAATAGCAAGTT | TGGAGGAAGCAGGG | CGTGCTCA | | CGCGGTTCTATCTA | TTTTTT |
| (tevoPreQ1) | CTGAGCAC | AAAATAAGGCTAGTCCGTTATCAAC | CTTCCTTTCCTCTGCC | GTCTG | | GTTACGCGTTAAA | |
| | GTGA | TTGAAAAAGTGGCACCGAGTCGGT | ATCAATAACTTCGTA | | | CCAACTAGAA | |
| | | GC | TAATGTATGCTATAC | | | | |
| | | | GAAGTTATAACAAT | | | | |
| CCR5, attB ins., twinPE | GCTGTGTTT | GTTTTAGAGCTAGAAATAGCAAGTT | ACGACGGAGACCGC | AGAGACGC | | | TTTTTT |
| pegRNA1 | GCGTCTCTC | AAAATAAGGCTAGTCCGTTATCAAC | CGTCGTCGACAAGCC | AAA | | | |
| | CC | TTGAAAAAGTGGCACCGAGTCGGT | | | | | |
| | | GC | | | | | |
| CCR5, attB ins., twinPE | GTATGGAA | GTTTTAGAGCTAGAAATAGCAAGTT | ACGACGGCGGTCTCC | GCTCTCATT | | | TTTTTT |
| pegRNA2 | AATGAGAG | AAAATAAGGCTAGTCCGTTATCAAC | GTCGTCAGGATCAT | TTC | | | |
| | CTGC | TTGAAAAAGTGGCACCGAGTCGGT | | | | | |
| | | GC | | | | | |
| PAH twinPE recode, | GTGGTTTCC | GTTTTAGAGCTAGAAATAGCAAGTT | ACGCGGAATGCGAG | GTCGGAGG | TCTCTCT | CGCGGTTCTATCTA | TTTTTT |
| pegRNA1 | GCCTCCGA | AAAATAAGGCTAGTCCGTTATCAAC | ACCCCCCAGAAAGTC | CG | CTTGA | GTTACGCGTTAAA | |
| | CCTG | TTGAAAAAGTGGCACCGAGTCGGT | TCTACTGCTCAAGAG | | | CCAACTAGAAA | |
| | | GC | CCCGGCAACGG | | | | |
| PAH twinPE recode, | GTCTGATG | GTTTTAGAGCTAGAAATAGCAAGTT | GGGCTCTTGAGCAGT | CACACAGT | CTCTCTC | CGCGGTTCTATCTA | TTTTTT |
| pegRNA2 | TACTGTGT | AAAATAAGGCTAGTCCGTTATCAAC | AGAGACTTTCTGGGG | ACAT | TTGA | GTTACGCGTTAAA | |
| | GCAG | TTGAAAAAGTGGCACCGAGTCGGT | GGTCTCGCATTCCGC | | | CCAACTAGAAA | |
| | | GC | GTGTTTCATTG | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| HEK3, + 1 FLAG ins | GGCCCAGA | GTTTTAGAGCTAGAAATAGCAAGTT | TGGAGGAAGCAGGG | CGTGCTCA | TCTCTCT | GGGTCAGGAGCCC | TTTTTT |
| (mpknot) | CTGAGCAC | AAAATAAGGCTAGTCCGTTATCAAC | CTTCCTTTCCTCTGCC | GTCTG | C | CCCCCCTGAACCC | |
| | GTGA | TTGAAAAAGTGGCACCGAGTCGGT | ATCACTTATCGTCGT | | | AGGATAACCCTCA | |
| | | GC | CATCCTTGTAATC | | | AAGTCGGGGGGCA | |
| | | | | | | ACCC | |
| HEK3, +1 loxP ins | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCAATAACTTCGTA | | | | |
| | | GC | TAATGTATGCTATAC | | | | |
| | | | GAAGTTATAACAAT | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| HEK3, +1 loxP ins | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCAATAACTTCGTA | | | | |
| | | GC | TAATGTATGCTATAC | | | | |
| | | | GAAGTTATAACAAT | | | | |
| HEK3 large hairpin | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCAAAGCCCTGCTT | | | | |
| | | GC | CCTCCA | | | | |
| HEK3 unpinned | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCAAAGCACTGGAT | | | | |
| | | GC | CCTCCA | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| HEK3 large hairpin | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCAAAGCCCTGCTT | | | | |
| | | GC | CCTCCA | | | | |
| HEK3 large hairpin UC | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCAAAGCACTGGAT | | | | |
| | | GC | CCTCCA | | | | |
| HEK3 small hairpin | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCATTCCTCCA | | | | |
| | | GC | | | | | |
| HEK3 med hairpin | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCACTGCTTCCTCC | | | | |
| | | GC | A | | | | |
| HEK3 med hairpin UC | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCACTGGTTCCTGC | | | | |
| | | GC | A | | | | |
| HEK3 med hairpin 2 | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCAGCTTCCTCCA | | | | |
| | | GC | | | | | |
| HEK3 install hairpin | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCGGC | GTCTG | | | |
| | GTGA | TTGAAAAAGTGGCACCGAGTCGGT | ACAATTCAGGTCAGG | | | | |
| | | GC | TGCCGCTGCCATCA | | | | |
| HEK3 install hairpin UC | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCGGA | GTCTG | | | |
| | GTGA | TTGAAAAAGTGGCACCGAGTCGGT | ACAATTCAGGTCAGG | | | | |
| | | GC | TGACGCTGCCATCA | | | | |
| RNF2 large hairpin | GTCATCTTA | GTTTTAGAGCTAGAAATAGCAAGTT | TAAGCAAAACATGG | GTAATGAC | | | TTTTTT |
| | GTCATTAC | AAAATAAGGCTAGTCCGTTATCAAC | GAACTCAGTTTATAT | TAAGATG | | | |
| | CTG | TTGAAAAAGTGGCACCGAGTCGGT | GAGTTACAACGAACT | | | | |
| | | GC | AACTCATATAAACTG | | | | |
| | | | AGTTCCCATGTTACC | | | | |
| | | | TCAG | | | | |
| RNF2 med hairpin | GTCATCTTA | GTTTTAGAGCTAGAAATAGCAAGTT | TAAGCAAAACATGG | GTAATGAC | | | TTTTTT |
| | GTCATTAC | AAAATAAGGCTAGTCCGTTATCAAC | GAACTCAGTTTATAT | TAAGATG | | | |
| | CTG | TTGAAAAAGTGGCACCGAGTCGGT | GAGTTACAACGAACT | | | | |
| | | GC | AACTGACATATACTG | | | | |
| | | | AGTGACCATGTTACC | | | | |
| | | | TCAG | | | | |
| RNF2 med hairpin 2 | GTCATCTTA | GTTTTAGAGCTAGAAATAGCAAGTT | TAAGCAAAACATGG | GTAATGAC | | | TTTTTT |
| | GTCATTAC | AAAATAAGGCTAGTCCGTTATCAAC | GAACTCAGTTTATAT | TAAGATG | | | |
| | CTG | TTGAAAAAGTGGCACCGAGTCGGT | GAGTTACAACGAACA | | | | |
| | | GC | CCATAACTTCGTATA | | | | |
| | | | ATGTATGCTATACGA | | | | |
| | | | AGTTATAACAATTCA | | | | |
| | | | G | | | | |
| HEK3, +1 loxP ins | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCAATAACTTCGTA | | | | |
| | | GC | TAATGTATGCTATAC | | | | |
| | | | GAAGTTATAACAAT | | | | |
| DNMT1 large hairpin | GATTCCTG | GTTTTAGAGCTAGAAATAGCAAGTT | AGGAGGAAGCTGCT | TTCTGGCA | | | TTTTTT |
| | GTGCCAGA | AAAATAAGGCTAGTCCGTTATCAAC | AAGGACTAGTTCTGC | CCAGGA | | | |
| | AACA | | CAACTAGTCCTTAGC | | | | |
| | | TTGAAAAAGTGGCACCGAGTCGGT | ACTCCCGTCACCCCT | | | | |
| | | GC | GT | | | | |
| DNMT1 med hairpin | GATTCCTG | GTTTTAGAGCTAGAAATAGCAAGTT | AGGAGGAAGCTGCT | TTCTGGCA | | | TTTTTT |
| | GTGCCAGA | AAAATAAGGCTAGTCCGTTATCAAC | AAGGACTAGTTCTGC | CCAGGA | | | |
| | AACA | TTGAAAAAGTGGCACCGAGTCGGT | CCTAGTCCTTAGCAG | | | | |
| | | GC | TCCCGTCACCCCTGT | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| DNMT1, nicking sgRNA | GCCCTTCA | GTTTTAGAGCTAGAAATAGCAAGTT | | | | | TTTTTT |
| | GCTAAAAT | AAAATAAGGCTAGTCCGTTATCAAC | | | | | |
| | AAAGG | TTGAAAAAGTGGCACCGAGTCGGT | | | | | |
| | | GC | | | | | |
| IDS edit 1-twinPE-pegRNA1 | GCACTCAT | GTTTTAGAGCTAGAAATAGCAAGTT | GTCTGGTCAACCACC | CTTGGAGG | | | TTTTTT |
| | TTCCTCCAA | AAAATAAGGCTAGTCCGTTATCAAC | GCGGTCTCAGTGGTG | AAA | | | |
| | GCTC | TTGAAAAAGTGGCACCGAGTCGGT | TACGGTACAAACCT | | | | |
| | | GC | | | | | |
| IDS edit 1-twinPE-pegRNA2 | GTTATGGTT | GTTTTAGAGCTAGAAATAGCAAGTT | TACCGTACACCACTG | ATGGAGTA | | | TTTTTT |
| | TACTCCATC | AAAATAAGGCTAGTCCGTTATCAAC | AGACCGCGGTGGTTG | AACC | | | |
| | TA | TTGAAAAAGTGGCACCGAGTCGGT | ACCAGACAAACCT | | | | |
| | | GC | | | | | |
| IDS edit 2-twinPE-pegRNA1 | GTTTTGGTT | GTTTTAGAGCTAGAAATAGCAAGTT | GGCTTGTCGACGACG | ATAGGGTA | | | TTTTTT |
| | TACCCTATC | AAAATAAGGCTAGTCCGTTATCAAC | GCGGTCTCCGTCGTC | AACCA | | | |
| | TA | TTGAAAAAGTGGCACCGAGTCGGT | AGGATCAT | | | | |
| | | GC | | | | | |
| IDS edit 2-twinPE-pegRNA2 | GTGCCACC | GTTTTAGAGCTAGAAATAGCAAGTT | ATGATCCTGACGACG | CTCACTGTT | | | TTTTTT |
| | TAACAGTG | AAAATAAGGCTAGTCCGTTATCAAC | GAGACCGCCGTCGTC | AGGT | | | |
| | AGCTG | TTGAAAAAGTGGCACCGAGTCGGT | GACAAGCC | | | | |
| | | GC | | | | | |
| CCR5 edit 1-twinPE- | GACCCCTC | GTTTTAGAGCTAGAAATAGCAAGTT | GGCTTGTCGACGACG | TGAAATAC | | | TTTTTT |
| pegRNA1 | AGTATTTC | AAAATAAGGCTAGTCCGTTATCAAC | GCGGTCTCCGTCGTC | TG | | | |
| | AGCT | TTGAAAAAGTGGCACCGAGTCGGT | AGGATCAT | | | | |
| | | GC | | | | | |
| CCR5 edit 1-twinPE- | GAAAAGAC | GTTTTAGAGCTAGAAATAGCAAGTT | ATGATCCTGACGACG | GTGCTTGA | | | TTTTTT |
| pegRNA2 | ATCAAGCA | AAAATAAGGCTAGTCCGTTATCAAC | GAGACCGCCGTCGTC | TGTC | | | |
| | CAGA | TTGAAAAAGTGGCACCGAGTCGGT | GACAAGCC | | | | |
| | | GC | | | | | |
| CCR5 edit 2-twinPE- | GAAGTGTG | GTTTTAGAGCTAGAAATAGCAAGTT | ATGATCCTGACGACG | CCCAAGTG | | CGCGGTTCTATCTA | TTTTTT |
| pegRNA1 | ATCACTTG | AAAATAAGGCTAGTCCGTTATCAAC | GAGACCGCCGTCGTC | ATC | | GTTACGCGTTAAA | |
| | GGTGG | TTGAAAAAGTGGCACCGAGTCGGT | GACAAGCC | | | CCAACTAGAA | |
| | | GC | | | | | |
| CCR5 edit 2-twinPE- | GTATGGAA | GTTTTAGAGCTAGAAATAGCAAGTT | GGCTTGTCGACGACG | GCTCTCATT | | CGCGGTTCTATCTA | TTTTTT |
| pegRNA2 | AATGAGAG | AAAATAAGGCTAGTCCGTTATCAAC | GCGGTCTCCGTCGTC | TTC | | GTTACGCGTTAAA | |
| | CTGC | TTGAAAAAGTGGCACCGAGTCGGT | AGGATCAT | | | CCAACTAGAA | |
| | | GC | | | | | |
| CCR5 edit 3-twinPE- | CACAGTCT | GTTTTAGAGCTAGAAATAGCAAGTT | ATGATCCTGACGACG | GTCTGGGT | | | TTTTTT |
| pegRNA1 | CACCCAGA | AAAATAAGGCTAGTCCGTTATCAAC | GAGACCGCCGTCGTC | GAGAC | | | |
| | CTCC | TTGAAAAAGTGGCACCGAGTCGGT | GACAAGCC | | | | |
| | | GC | | | | | |
| CCR5 edit 3-twinPE- | GTATTTCA | GTTTTAGAGCTAGAAATAGCAAGTT | GGCTTGTCGACGACG | CATCCCAG | | | TTTTTT |
| pegRNA2 | GCTGGGAT | AAAATAAGGCTAGTCCGTTATCAAC | GCGGTCTCCGTCGTC | CT | | | |
| | GGGA | TTGAAAAAGTGGCACCGAGTCGGT | AGGATCAT | | | | |
| | | GC | | | | | |
| Rosa26, attB insertion- | GCTACTGTT | GTTTTAGAGCTAGAAATAGCAAGTT | ATGATCCTGACGACG | TTAGAGTG | CAGTAG | CGCGGTTCTATCTA | TTTTTT |
| twinPE-pegRNA1 | CACTCTAA | AAAATAAGGCTAGTCCGTTATCAAC | GAGACCGCCGTCGTC | AA | | GTTACGCGTTAAA | |
| | CAG | TTGAAAAAGTGGCACCGAGTCGGT | GACAAGCC | | | CCAACTAGAA | |
| | | GC | | | | | |
| Rosa26, attB insertion- | GAATCTGC | GTTTTAGAGCTAGAAATAGCAAGTT | GGCTTGTCGACGACG | GATATACT | CAGATT | CGCGGTTCTATCTA | TTTTTT |
| twinPE-pegRNA2 | TAGTATAT | AAAATAAGGCTAGTCCGTTATCAAC | GCGGTCTCCGTCGTC | AG | | GTTACGCGTTAAA | |
| | CCGT | TTGAAAAAGTGGCACCGAGTCGGT | AGGATCAT | | | CCAACTAGAA | |
| | | GC | | | | | |
| mDnmt1, loxP insertion | GCGGGCTG | GTTTTAGAGCTAGAAATAGCAAGTT | GAGCTGTTCTGTCGT | CGAACAGC | | CGCGGTTCTATCTA | TTTTTT |
| pegRNA, tevoPreQ1 | GAGCTGTT | AAAATAAGGCTAGTCCGTTATCAAC | CTGCAACCTGCAAGA | T | | GTTACGCGTTAAA | |
| | CGCGC | TTGAAAAAGTGGCACCGAGTCGGT | TGCCAGCGATTGTTA | | | CCAACTAGAA | |
| | | GC | TAACTTCGTATAGCA | | | | |
| | | | TACATTATACGAAGT | | | | |
| | | | TATCG | | | | |
| mDnmt1, nicking sgRNA | GCCGCGCG | GTTTTAGAGCTAGAAATAGCAAGTT | | | | | |
| | CGCGAAAA | AAAATAAGGCTAGTCCGTTATCAAC | | | | | |
| | AGCCG | TTGAAAAAGTGGCACCGAGTCGGT | | | | | |
| | | GC | | | | | |
| PAH, exon 7 recode pair 1 | GTGGTTTCC | GTTTTAGAGCTAGAAATAGCAAGTT | AGAAAGTCTCTACTG | GTCGGAGG | TCTCTCT | TTGACGCGGTTCT | TTTTTT |
| twinPE-pegRNA1 | GCCTCCGA | AAAATAAGGCTAGTCCGTTATCAAC | CTCAAGAGCCCGGCA | CG | C | ATCTAGTTACGCG | |
| | CCTG | TTGAAAAAGTGGCACCGAGTCGGT | ACGG | | | TTAAACCAACTAG | |
| | | GC | | | | AAA | |
| PAH, exon 7 recode pair 1 | GAGTGGAA | GTTTTAGAGCTAGAAATAGCAAGTT | TCTTGAGCAGTAGAG | CTTCCGAG | TCTCTCT | TTGACGCGGTTCT | TTTTTT |
| twinPE-pegRNA2 | GACTCGGA | AAAATAAGGCTAGTCCGTTATCAAC | ACTTTCTGGGGGGTC | TCTTC | C | ATCTAGTTACGCG | |
| | AGGCC | TTGAAAAAGTGGCACCGAGTCGGT | TCGC | | | TTAAACCAACTAG | |
| | | GC | | | | AAA | |
| PAH, exon 7 recode pair 2 | GTGGTTTCC | GTTTTAGAGCTAGAAATAGCAAGTT | GAGACCCCCCAGAA | GTCGGAGG | TCTCTCT | TTGACGCGGTTCT | TTTTTT |
| twinPE-pegRNA-1 | GCCTCCGA | AAAATAAGGCTAGTCCGTTATCAAC | AGTCTCTACTGCTCA | CG | C | ATCTAGTTACGCG | |
| | CCTG | TTGAAAAAGTGGCACCGAGTCGGT | AGAGCCCGGCAACG | | | TTAAACCAACTAG | |
| | | GC | G | | | AAA | |
| PAH, exon 7 recode pair 2 | GAGTGGAA | GTTTTAGAGCTAGAAATAGCAAGTT | GTTGCCGGGCTCTTG | CTTCCGAG | TCTCTCT | TTGACGCGGTTCT | TTTTTT |
| twinPE-pegRNA-2 | GACTCGGA | AAAATAAGGCTAGTCCGTTATCAAC | AGCAGTAGAGACTTT | TCTTC | C | ATCTAGTTACGCG | |
| | AGGCC | TTGAAAAAGTGGCACCGAGTCGGT | CTGGGGGGTCTCGC | | | TTAAACCAACTAG | |
| | | GC | | | | AAA | |
| CCR5, FKBP ins-twinPE- | GGTACCTA | GTTTTAGAGCTAGAAATAGCAAGTT | ACCTTCCCCAAGCGC | GACAATCG | | | TTTTTT |
| pegRNA1 | TCGATTGTC | AAAATAAGGCTAGTCCGTTATCAAC | GGCCAGACCTGCGTG | AT | | | |
| | AGG | TTGAAAAAGTGGCACCGAGTCGGT | GTGCACTACACCGGG | | | | |
| | | GC | ATGCTTGAAGATGGA | | | | |
| | | | AAGAAATTT | | | | |
| CCR5, FKBP ins-twinPE- | GCTCACTA | GTTTTAGAGCTAGAAATAGCAAGTT | ACCACGCAGGTCTGG | GGCGGCAG | | | TTTTTT |
| pegRNA2 | TGCTGCCG | AAAATAAGGCTAGTCCGTTATCAAC | CCGCGCTTGGGGAAG | CAT | | | |
| | CCCAG | TTGAAAAAGTGGCACCGAGTCGGT | GTGCGCCCGTCTCCT | | | | |
| | | GC | GGGGAGATGGTTTCC | | | | |
| | | | ACCTGCACTCC | | | | |
| Rosa26, attB insertion- | GCTACTGTT | GTTTTAGAGCTAGAAATAGCAAGTT | ATGATCCTGACGACG | TTAGAGTG | CAGTAG | CGCGGTTCTATCTA | TTTTTT |
| twinPE-pegRNA1 | CACTCTAA | AAAATAAGGCTAGTCCGTTATCAAC | GAGACCGCCGTCGTC | AA | | GTTACGCGTTAAA | |
| | CAG | TTGAAAAAGTGGCACCGAGTCGGT | GACAAGCC | | | CCAACTAGAA | |
| | | GC | | | | | |
| Rosa26, attB insertion- | GAATCTGC | GTTTTAGAGCTAGAAATAGCAAGTT | GGCTTGTCGACGACG | GATATACT | CAGATT | CGCGGTTCTATCTA | TTTTTT |
| twinPE-pegRNA2 | TAGTATAT | AAAATAAGGCTAGTCCGTTATCAAC | GCGGTCTCCGTCGTC | AG | | GTTACGCGTTAAA | |
| | CCGT | TTGAAAAAGTGGCACCGAGTCGGT | AGGATCAT | | | CCAACTAGAA | |
| | | GC | | | | | |
| IDS attP-twinPE-pegRNA1 | GCACTCAT | GTTTTAGAGCTAGAAATAGCAAGTT | GTCTGGTCAACCACC | CTTGGAGG | | | TTTTTT |
| | TTCCTCCAA | AAAATAAGGCTAGTCCGTTATCAAC | GCGGTCTCAGTGGTG | AAA | | | |
| | GCTC | TTGAAAAAGTGGCACCGAGTCGGT | TACGGTACAAACCT | | | | |
| | | GC | | | | | |
| IDS attP-twinPE-pegRNA2 | GTTATGGTT | GTTTTAGAGCTAGAAATAGCAAGTT | TACCGTACACCACTG | ATGGAGTA | | | TTTTTT |
| | TACTCCATC | AAAATAAGGCTAGTCCGTTATCAAC | AGACCGCGGTGGTTG | AACC | | | |
| | TA | TTGAAAAAGTGGCACCGAGTCGGT | ACCAGACAAACCT | | | | |
| | | GC | | | | | |
| CCR5 attB-twinPE-pegRNA1 | GACCCCTC | GTTTTAGAGCTAGAAATAGCAAGTT | GGCTTGTCGACGACG | TGAAATAC | | | TTTTTT |
| | AGTATTTC | AAAATAAGGCTAGTCCGTTATCAAC | GCGGTCTCCGTCGTC | TG | | | |
| | AGCT | TTGAAAAAGTGGCACCGAGTCGGT | AGGATCAT | | | | |
| | | GC | | | | | |
| CCR5 attB-twinPE-pegRNA2 | GAAAAGAC | GTTTTAGAGCTAGAAATAGCAAGTT | ATGATCCTGACGACG | GTGCTTGA | | | TTTTTT |
| | ATCAAGCA | AAAATAAGGCTAGTCCGTTATCAAC | GAGACCGCCGTCGTC | TGTC | | | |
| | CAGA | TTGAAAAAGTGGCACCGAGTCGGT | GACAAGCC | | | | |
| | | GC | | | | | |
| HEK3, + 1 FLAG ins | GGCCCAGA | GTTTTAGAGCTAGAAATAGCAAGTT | TGGAGGAAGCAGGG | CGTGCTCA | TCTCTCT | GGGTCAGGAGCCC | TTTTTT |
| (mpknot) | CTGAGCAC | AAAATAAGGCTAGTCCGTTATCAAC | CTTCCTTTCCTCTGCC | GTCTG | C | CCCCCCTGAACCC | |
| | GTGA | TTGAAAAAGTGGCACCGAGTCGGT | ATCACTTATCGTCGT | | | AGGATAACCCTCA | |
| | | GC | CATCCTTGTAATC | | | AAGTCGGGGGGCA | |
| | | | | | | ACCC | |
| DNMT1, 1-15 deletion | GATTCCTG | GTTTTAGAGCTAGAAATAGCAAGTT | AGGAGGAAGCTGCT | TTCTGGCA | CCTCTTC | TTGACGCGGTTCT | TTTTTT |
| (evopreQ1) | GTGCCAGA | AAAATAAGGCTAGTCCGTTATCAAC | AAGGACTAGTTCTGC | CCAGGA | T | ATCTAGTTACGCG | |
| | AACA | TTGAAAAAGTGGCACCGAGTCGGT | CC | | | TTAAACCAACTAG | |
| | | GC | | | | AAA | |
| RNF2, 1-15 deletion | GTCATCTTA | GTTTTAGAGCTAGAAATAGCAAGTT | TAAGCAAAACATGG | GTAATGAC | TCATCTC | TTGACGCGGTTCT | TTTTTT |
| (evopreQ1) | GTCATTAC | AAAATAAGGCTAGTCCGTTATCAAC | GAACTCAGTTTATAT | TAAGATG | T | ATCTAGTTACGCG | |
| | CTG | TTGAAAAAGTGGCACCGAGTCGGT | GAGTTA | | | TTAAACCAACTAG | |
| | | GC | | | | AAA | |
| EMX1, 1-15 deletion | GAGTCCGA | GTTTTAGAGCTAGAAATAGCAAGTT | TTGGCCTGCTTCGTG | TTCTTCTGC | AACAATC | TTGACGCGGTTCT | TTTTTT |
| (evopreQ1) | GCAGAAGA | AAAATAAGGCTAGTCCGTTATCAAC | GCAATGCGCCACCGG | TCGGA | T | ATCTAGTTACGCG | |
| | AGAA | TTGAAAAAGTGGCACCGAGTCGGT | TTGATG | | | TTAAACCAACTAG | |
| | | GC | | | | AAA | |
| EMX1, +5 G to T (evopreQ1) | GAGTCCGA | GTTTTAGAGCTAGAAATAGCAAGTT | GTGATGGGAGCACTT | TTCTTCTGC | AACAATC | TTGACGCGGTTCT | TTTTTT |
| | GCAGAAGA | AAAATAAGGCTAGTCCGTTATCAAC | C | TCGGA | T | ATCTAGTTACGCG | |
| | AGAA | TTGAAAAAGTGGCACCGAGTCGGT | | | | TTAAACCAACTAG | |
| | | GC | | | | AAA | |
| RUNX1, +5 G to T | GCATTTTCA | GTTTTAGAGCTAGAAATAGCAAGTT | AGGAAATGACTCAA | CTTCCTCCT | AACTCTC | TTGACGCGGTTCT | TTTTTT |
| (evopreQ1) | GGAGGAAG | AAAATAAGGCTAGTCCGTTATCAAC | ATATGCTGTCTGAAG | GAAAAT | T | ATCTAGTTACGCG | |
| | CGA | TTGAAAAAGTGGCACCGAGTCGGT | CAATCG | | | TTAAACCAACTAG | |
| | | GC | | | | AAA | |
| VEGFA, +5 G to T | GATGTCTG | GTTTTAGAGCTAGAAATAGCAAGTT | AATGTGCCATCTGGA | TCTGGCCT | ACAATCT | TTGACGCGGTTCT | TTTTTT |
| (evopreQ1) | CAGGCCAG | AAAATAAGGCTAGTCCGTTATCAAC | GCACTCA | GCAGA | C | ATCTAGTTACGCG | |
| | ATGA | TTGAAAAAGTGGCACCGAGTCGGT | | | | TTAAACCAACTAG | |
| | | GC | | | | AAA | |
| RNF2, +5 G to T | GTCATCTTA | GTTTTAGAGCTAGAAATAGCAAGTT | AACGAACACATCAG | GTAATGAC | | | TTTTTT |
| | GTCATTAC | AAAATAAGGCTAGTCCGTTATCAAC | | TAAGATG | | | |
| | CTG | TTGAAAAAGTGGCACCGAGTCGGT | | | | | |
| | | GC | | | | | |
| FANCF, +5 G to T (mpknot) | GGAATCCC | GTTTTAGAGCTAGAAATAGCAAGTT | GGAAAAGCGATCAA | GCTGCAGA | CAATCAC | GGGTCAGGAGCCC | TTTTTT |
| | TTCTGCAG | AAAATAAGGCTAGTCCGTTATCAAC | GGT | AGGGA | T | CCCCCCTGAACCC | |
| | CACC | TTGAAAAAGTGGCACCGAGTCGGT | | | | AGGATAACCCTCA | |
| | | GC | | | | AAGTCGGGGGGCA | |
| | | | | | | ACCC | |
| PRNP, +6 G to T (evopreQ1) | GCAGTGGT | GTTTTAGAGCTAGAAATAGCAAGTT | ATGTAGACGCCA | AGGCCCCC | TAGACAC | CGCGGTTCTATCTA | TTTTTT |
| | GGGGGGCC | AAAATAAGGCTAGTCCGTTATCAAC | | CACC | A | GTTACGCGTTAAA | |
| | TTGG | TTGAAAAAGTGGCACCGAGTCGGT | | | | CCAACTAGAA | |
| | | GC | | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| DNMT1, nicking sgRNA | GCCCTTCA | GTTTTAGAGCTAGAAATAGCAAGTT | | | | | TTTTTT |
| | GCTAAAAT | AAAATAAGGCTAGTCCGTTATCAAC | | | | | |
| | AAAGG | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| EMX1, nicking sgRNA | GCCGTTTGT | GTTTTAGAGCTAGAAATAGCAAGTT | | | | | TTTTTT |
| | ACTTTGTCC | AAAATAAGGCTAGTCCGTTATCAAC | | | | | |
| | TC | TTGAAAAAGTGGCACCGAGTCGGT | | | | | |
| | | GC | | | | | |
| RUNX1, nicking sgRNA | GATGAAGC | GTTTTAGAGCTAGAAATAGCAAGTT | | | | | TTTTTT |
| | ACTGTGGG | AAAATAAGGCTAGTCCGTTATCAAC | | | | | |
| | TACGA | TTGAAAAAGTGGCACCGAGTCGGT | | | | | |
| | | GC | | | | | |
| VEGFA, nicking sgRNA | GAGCCCAG | GTTTTAGAGCTAGAAATAGCAAGTT | | | | | TTTTTT |
| | GGCTGGGC | AAAATAAGGCTAGTCCGTTATCAAC | | | | | |
| | ACAG | TTGAAAAAGTGGCACCGAGTCGGT | | | | | |
| | | GC | | | | | |
| FANCF, nicking sgRNA | GGGGTCCC | GTTTTAGAGCTAGAAATAGCAAGTT | | | | | TTTTTT |
| | AGGTGCTG | AAAATAAGGCTAGTCCGTTATCAAC | | | | | |
| | ACGT | TTGAAAAAGTGGCACCGAGTCGGT | | | | | |
| | | GC | | | | | |
| PRNP, nicking sgRNA | GCATGTTTT | GTTTTAGAGCTAGAAATAGCAAGTT | | | | | TTTTTT |
| | CACGATAG | AAAATAAGGCTAGTCCGTTATCAAC | | | | | |
| | TAA | TTGAAAAAGTGGCACCGAGTCGGT | | | | | |
| | | GC | | | | | |
| UGT1A1 correction, pegRNA | GCTCTAGG | GTTTTAGAGCTAGAAATAGCAAGTT | ACAATTCCATGTTCT | CTTCAAATT | | CGCGGTTCTATCTA | TTTTTT |
| | AATTTGAA | AAAATAAGGCTAGTCCGTTATCAAC | CCAGAAGCATTAATG | CCTA | | GTTACGCGTTAAA | |
| | GCCA | TTGAAAAAGTGGCACCGAGTCGGT | TAGG | | | CCAACTAGAA | |
| | | GC | | | | | |
| UGT1A1 correction, nicking | GATTGCCA | GTTTTAGAGCTAGAAATAGCAAGTT | | | | | TTTTTT |
| sgRNA | TAGCTTTCT | AAAATAAGGCTAGTCCGTTATCAAC | | | | | |
| | TCTC | TTGAAAAAGTGGCACCGAGTCGGT | | | | | |
| | | GC | | | | | |
| GAA correction, pegRNA | GTCGTTGTC | GTTTTAGAGCTAGAAATAGCAAGTT | TCCTCCCACCAGGCC | CCATACCT | | CGCGGTTCTATCTA | TTTTTT |
| | CAGGTATG | AAAATAAGGCTAGTCCGTTATCAAC | AGGGCTGTGGGGTTG | GGA | | GTTACGCGTTAAA | |
| | GCCC | TTGAAAAAGTGGCACCGAGTCGGT | GTGAAGTCGGGGAA | | | CCAACTAGAA | |
| | | GC | GGCAGTGGAGCCGG | | | | |
| | | | G | | | | |
| GAA correction, nicking | GAGCCACC | GTTTTAGAGCTAGAAATAGCAAGTT | | | | | TTTTTT |
| sgRNA | ATGTCCTCC | AAAATAAGGCTAGTCCGTTATCAAC | | | | | |
| | CACC | TTGAAAAAGTGGCACCGAGTCGGT | | | | | |
| | | GC | | | | | |
| RECQL3 correction, pegRNA | GTCTGAGT | GTTTTAGAGCTAGAAATAGCAAGTT | TCCAGCTACATATCT | GATAAGAC | | CGCGGTTCTATCTA | TTTTTT |
| | CAGTCTTAT | AAAATAAGGCTAGTCCGTTATCAAC | GACAGGT | TG | | GTTACGCGTTAAA | |
| | CACC | TTGAAAAAGTGGCACCGAGTCGGT | | | | CCAACTAGAA | |
| | | GC | | | | | |
| RECQL3 correction, nicking | GATTCCAG | GTTTTAGAGCTAGAAATAGCAAGTT | | | | | TTTTTT |
| sgRNA | CTACATAT | AAAATAAGGCTAGTCCGTTATCAAC | | | | | |
| | CTGAC | TTGAAAAAGTGGCACCGAGTCGGT | | | | | |
| | | GC | | | | | |
| EMX1, +5 G to T | GAGTCCGA | GTTTTAGAGCTAGAAATAGCAAGTT | ATGGGAGCACTTC | TTCTTCTGC | | | TTTTTT |
| | GCAGAAGA | AAAATAAGGCTAGTCCGTTATCAAC | | TCGG | | | |
| | AGAA | TTGAAAAAGTGGCACCGAGTCGGT | | | | | |
| | | GC | | | | | |
| HEK3, +26 C to G | GGCCCAGA | GTTTTAGAGCTAGAAATAGCAAGTT | TGGAGGAACCAGGG | CGTGCTCA | | | TTTTTT |
| | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCA | | | | |
| | | GC | | | | | |
| HEK3, +1 CTT ins | GGCCCAGA | GTTTTAGAGCTAGAAATAGCAAGTT | TCTGCCATCAAAG | CGTGCTCA | | | TTTTTT |
| | CTGAGCAC | AAAATAAGGCTAGTCCGTTATCAAC | | GTCTG | | | |
| | GTGA | TTGAAAAAGTGGCACCGAGTCGGT | | | | | |
| | | GC | | | | | |
| RNF2, +5 G to T | GTCATCTTA | GTTTTAGAGCTAGAAATAGCAAGTT | AACGAACACATCAG | GTAATGAC | | | TTTTTT |
| | GTCATTAC | AAAATAAGGCTAGTCCGTTATCAAC | | TAAGATG | | | |
| | CTG | TTGAAAAAGTGGCACCGAGTCGGT | | | | | |
| | | GC | | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| EMX1, nicking sgRNA | GCCGTTTGT | GTTTTAGAGCTAGAAATAGCAAGTT | | | | | TTTTTT |
| | ACTTTGTCC | AAAATAAGGCTAGTCCGTTATCAAC | | | | | |
| | TC | TTGAAAAAGTGGCACCGAGTCGGT | | | | | |
| | | GC | | | | | |
| Pcsk9, +3 C to G, +6 G to C | GCCAGGTT | GTTTTAGAGCTAGAAATAGCAAGTT | TTTGTCTTCGCGCAC | CATCCCAT | TTTAAAT | CGCGGTTCTATCTA | TTTTTT |
| | CCATGGGA | AAAATAAGGCTAGTCCGTTATCAAC | AG | GG | A | GTTACGCGTTAAA | |
| | TGCTC | TTGAAAAAGTGGCACCGAGTCGGT | | | | CCAACTAGAA | |
| | | GC | | | | | |
| RNF2, +5 G to T | GTCATCTTA | GTTTTAGAGCTAGAAATAGCAAGTT | AACGAACACATCAG | GTAATGAC | | | TTTTTT |
| | GTCATTAC | AAAATAAGGCTAGTCCGTTATCAAC | | TAAGATG | | | |
| | CTG | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| Pcsk9, nicking sgRNA | GCATGGCT | GTTTTAGAGCTAGAAATAGCAAGTT | | | | | TTTTTT |
| | GTCTGGTTC | AAAATAAGGCTAGTCCGTTATCAAC | | | | | |
| | TGT | TTGAAAAAGTGGCACCGAGTCGGT | | | | | |
| | | GC | | | | | |
| Ctnnb1, +6 G to A | GAGGGTTG | GTTTTAGAGCTAGAAATAGCAAGTT | GCTCCTTTCCTGA | GTGGCAAG | | | TTTTTT |
| | CCCTTGCC | AAAATAAGGCTAGTCCGTTATCAAC | | GGCAA | | | |
| | ACTCA | TTGAAAAAGTGGCACCGAGTCGGT | | | | | |
| | | GC | | | | | |
| Chd2, +5 G to A | GCGGTAGC | GTTTTAGAGCTAGAAATAGCAAGTT | GATGCTCACC | GTTCTGGG | | | TTTTTT |
| | TCCCAGAA | AAAATAAGGCTAGTCCGTTATCAAC | | AGCTA | | | |
| | CGGT | TTGAAAAAGTGGCACCGAGTCGGT | | | | | |
| | | GC | | | | | |
| Col12a1, +2 A to C | GTGACTTC | GTTTTAGAGCTAGAAATAGCAAGTT | AATGGACCCATGG | TGGAACCA | | | TTTTTT |
| | CATGGTTC | AAAATAAGGCTAGTCCGTTATCAAC | | TGGAA | | | |
| | CACAA | TTGAAAAAGTGGCACCGAGTCGGT | | | | | |
| | | GC | | | | | |
| Dnmt1, +1 C to G | GCGGGCTG | GTTTTAGAGCTAGAAATAGCAAGTT | AAGATGCCAGCC | CGAACAGC | AAACAC | CGCGGTTCTATCTA | TTTTTT |
| | GAGCTGTT | AAAATAAGGCTAGTCCGTTATCAAC | | TCCAG | AC | GTTACGCGTTAAA | |
| | CGCGC | TTGAAAAAGTGGCACCGAGTCGGT | | | | CCAACTAGAA | |
| | | GC | | | | | |
| Pcsk9, +3 C to G, +6 G to C | GCCAGGTT | GTTTTAGAGCTAGAAATAGCAAGTT | TTTGTCTTCGCGCAC | CATCCCAT | TTTAAAT | CGCGGTTCTATCTA | TTTTTT |
| | CCATGGGA | AAAATAAGGCTAGTCCGTTATCAAC | AG | GG | A | GTTACGCGTTAAA | |
| | TGCTC | TTGAAAAAGTGGCACCGAGTCGGT | | | | CCAACTAGAA | |
| | | GC | | | | | |
| CXCR4, +5 G to C | GCAACCAC | GTTTTAGAGCTAGAAATAGCAAGTT | TGACCGCTTCTACGC | TGACTTGT | | | TTTTTT |
| | CCACAAGT | AAAATAAGGCTAGTCCGTTATCAAC | CAA | GGGTGGT | | | |
| | CATTG | TTGAAAAAGTGGCACCGAGTCGGT | | | | | |
| | | GC | | | | | |
| IL2RB, +1 T to A, +5 G to C | GCCAGGTG | GTTTTAGAGCTAGAAATAGCAAGTT | TCCCAAGCCTCCGAC | CTTTGAAA | | | TTTTTT |
| | TCTTTCAAA | AAAATAAGGCTAGTCCGTTATCAAC | TT | GACAC | | | |
| | GTAG | TTGAAAAAGTGGCACCGAGTCGGT | | | | | |
| | | GC | | | | | |
| PRNP, +6 G to T (evopreQ1) | GCAGTGGT | GTTTTAGAGCTAGAAATAGCAAGTT | ATGTAGACGCCA | AGGCCCCC | TAGACAC | CGCGGTTCTATCTA | TTTTTT |
| | GGGGGGCC | AAAATAAGGCTAGTCCGTTATCAAC | | CACC | A | GTTACGCGTTAAA | |
| | TTGG | TTGAAAAAGTGGCACCGAGTCGGT | | | | CCAACTAGAA | |
| | | GC | | | | | |
| HEK3, + 1 FLAG ins | GGCCCAGA | GTTTTAGAGCTAGAAATAGCAAGTT | TGGAGGAAGCAGGG | CGTGCTCA | TCTCTCT | GGGTCAGGAGCCC | TTTTTT |
| (mpknot) | CTGAGCAC | AAAATAAGGCTAGTCCGTTATCAAC | CTTCCTTTCCTCTGCC | GTCTG | C | CCCCCCTGAACCC | |
| | GTGA | TTGAAAAAGTGGCACCGAGTCGGT | ATCACTTATCGTCGT | | | AGGATAACCCTCA | |
| | | GC | CATCCTTGTAATC | | | AAGTCGGGGGGCA | |
| | | | | | | ACCC | |
| PAH, exon 4 recode-twinPE- | GCCCAAGA | GTTTTAGAGCTAGAAATAGCAAGTT | AGGATGATCAGCGTC | CTTGAATG | TCTCTCT | TTGACGCGGTTCT | TTTTTT |
| pegRNA-1 | ACCATTCA | AAAATAAGGCTAGTCCGTTATCAAC | AAGTTCGGCTCCGTA | GTTC | C | ATCTAGTTACGCG | |
| | AGAGC | TTGAAAAAGTGGCACCGAGTCGGT | GGACAAGATTTGATT | | | TTAAACCAACTAG | |
| | | GC | AGCGAACCTATCAAG | | | AAA | |
| | | | TT | | | | |
| PAH, exon 4 recode-twinPE- | GCTACGGG | GTTTTAGAGCTAGAAATAGCAAGTT | TTGATAGGTTCGCTA | GAGTCCAT | TCTCTCT | TTGACGCGGTTCT | TTTTTT |
| pegRNA-2 | CCATGGAC | AAAATAAGGCTAGTCCGTTATCAAC | ATCAAATCTTGTCCT | GGCC | C | ATCTAGTTACGCG | |
| | TCACA | TTGAAAAAGTGGCACCGAGTCGGT | ACGGAGCCGAACTTG | | | TTAAACCAACTAG | |
| | | GC | ACGCTGATCATCCTG | | | AAA | |
| | | | T | | | | |
| PAH, exon 7 recode-twinPE- | GTGGTTTCC | GTTTTAGAGCTAGAAATAGCAAGTT | GAGACCCCCCAGAA | GTCGGAGG | TCTCTCT | TTGACGCGGTTCT | TTTTTT |
| pegRNA-1 | GCCTCCGA | AAAATAAGGCTAGTCCGTTATCAAC | AGTCTCTACTGCTCA | CG | C | ATCTAGTTACGCG | |
| | CCTG | TTGAAAAAGTGGCACCGAGTCGGT | AGAGCCCGGCAACG | | | TTAAACCAACTAG | |
| | | GC | G | | | AAA | |
| PAH, exon 7 recode-twinPE- | GAGTGGAA | GTTTTAGAGCTAGAAATAGCAAGTT | GTTGCCGGGCTCTTG | CTTCCGAG | TCTCTCT | TTGACGCGGTTCT | TTTTTT |
| pegRNA-2 | GACTCGGA | AAAATAAGGCTAGTCCGTTATCAAC | AGCAGTAGAGACTTT | TCTTC | C | ATCTAGTTACGCG | |
| | AGGCC | TTGAAAAAGTGGCACCGAGTCGGT | CTGGGGGGTCTCGC | | | TTAAACCAACTAG | |
| | | GC | | | | AAA | |
| CCR5 attB installation | GCTGTGTTT | GTTTTAGAGCTAGAAATAGCAAGTT | ACGACGGAGACCGC | AGAGACGC | | | TTTTTT |
| twinPE-pegRNA1 | GCGTCTCTC | AAAATAAGGCTAGTCCGTTATCAAC | CGTCGTCGACAAGCC | AAA | | | |
| | CC | TTGAAAAAGTGGCACCGAGTCGGT | | | | | |
| | | GC | | | | | |
| CCR5 attB installation | GTATGGAA | GTTTTAGAGCTAGAAATAGCAAGTT | ACGACGGCGGTCTCC | GCTCTCATT | | | TTTTTT |
| twinPE-pegRNA2 | AATGAGAG | AAAATAAGGCTAGTCCGTTATCAAC | GTCGTCAGGATCAT | TTC | | | |
| | CTGC | TTGAAAAAGTGGCACCGAGTCGGT | | | | | |
| | | GC | | | | | |
| RNF2, 1-15 deletion | GTCATCTTA | GTTTTAGAGCTAGAAATAGCAAGTT | TAAGCAAAACATGG | GTAATGAC | TCATCTC | TTGACGCGGTTCT | TTTTTT |
| (evopreQ1) | GTCATTAC | AAAATAAGGCTAGTCCGTTATCAAC | GAACTCAGTTTATAT | TAAGATG | T | ATCTAGTTACGCG | |
| | CTG | TTGAAAAAGTGGCACCGAGTCGGT | GAGTTA | | | TTAAACCAACTAG | |
| | | GC | | | | AAA | |
| Dnmt1, nicking sgRNA | GCCGCGCG | GTTTTAGAGCTAGAAATAGCAAGTT | | | | | TTTTTT |
| | CGCGAAAA | AAAATAAGGCTAGTCCGTTATCAAC | | | | | |
| | AGCCG | TTGAAAAAGTGGCACCGAGTCGGT | | | | | |
| | | GC | | | | | |
| Ctnnb1, nicking sgRNA | GAAAAGCT | GTTTTAGAGCTAGAAATAGCAAGTT | | | | | TTTTTT |
| | GCTGTCAG | AAAATAAGGCTAGTCCGTTATCAAC | | | | | |
| | CCAC | TTGAAAAAGTGGCACCGAGTCGGT | | | | | |
| | | GC | | | | | |
| Chd2, nicking sgRNA | GACCATCA | GTTTTAGAGCTAGAAATAGCAAGTT | | | | | TTTTTT |
| | GTATGAGC | AAAATAAGGCTAGTCCGTTATCAAC | | | | | |
| | AGCAT | TTGAAAAAGTGGCACCGAGTCGGT | | | | | |
| | | GC | | | | | |
| Col12a1, nicking sgRNA | GCCTGAGC | GTTTTAGAGCTAGAAATAGCAAGTT | | | | | TTTTTT |
| | AGGCCACG | AAAATAAGGCTAGTCCGTTATCAAC | | | | | |
| | AACA | TTGAAAAAGTGGCACCGAGTCGGT | | | | | |
| | | GC | | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| PRNP, nicking sgRNA | GCATGTTTT | GTTTTAGAGCTAGAAATAGCAAGTT | | | | | TTTTTT |
| | CACGATAG | AAAATAAGGCTAGTCCGTTATCAAC | | | | | |
| | TAA | TTGAAAAAGTGGCACCGAGTCGGT | | | | | |
| | | GC | | | | | |
| CXCR4, nicking sgRNA | GCATCTTTG | GTTTTAGAGCTAGAAATAGCAAGTT | | | | | TTTTTT |
| | CCAACGTC | AAAATAAGGCTAGTCCGTTATCAAC | | | | | |
| | AGTG | TTGAAAAAGTGGCACCGAGTCGGT | | | | | |
| | | GC | | | | | |
| IL2RB, nicking sgRNA | GCTCCCTCC | GTTTTAGAGCTAGAAATAGCAAGTT | | | | | TTTTTT |
| | AAGTTGTC | AAAATAAGGCTAGTCCGTTATCAAC | | | | | |
| | CACG | TTGAAAAAGTGGCACCGAGTCGGT | | | | | |
| | | GC | | | | | |
| CXCR4, +5 G to C | GCAACCAC | GTTTTAGAGCTAGAAATAGCAAGTT | TGACCGCTTCTACGC | TGACTTGT | | | TTTTTT |
| | CCACAAGT | AAAATAAGGCTAGTCCGTTATCAAC | CAA | GGGTGGT | | | |
| | CATTG | TTGAAAAAGTGGCACCGAGTCGGT | | | | | |
| | | GC | | | | | |
| IL2RB, +1 T to A, +5 G to C | GCCAGGTG | GTTTTAGAGCTAGAAATAGCAAGTT | TCCCAAGCCTCCGAC | CTTTGAAA | | | TTTTTT |
| | TCTTTCAAA | AAAATAAGGCTAGTCCGTTATCAAC | TT | GACAC | | | |
| | GTAG | TTGAAAAAGTGGCACCGAGTCGGT | | | | | |
| | | GC | | | | | |
| CXCR4, nicking sgRNA | GCATCTTTG | GTTTTAGAGCTAGAAATAGCAAGTT | | | | | TTTTTT |
| | CCAACGTC | AAAATAAGGCTAGTCCGTTATCAAC | | | | | |
| | AGTG | TTGAAAAAGTGGCACCGAGTCGGT | | | | | |
| | | GC | | | | | |
| IL2RB, nicking sgRNA | GCTCCCTCC | GTTTTAGAGCTAGAAATAGCAAGTT | | | | | TTTTTT |
| | AAGTTGTC | AAAATAAGGCTAGTCCGTTATCAAC | | | | | |
| | CACG | TTGAAAAAGTGGCACCGAGTCGGT | | | | | |
| | | GC | | | | | |
| PAH, exon 4 recode-twinPE- | GCCCAAGA | GTTTTAGAGCTAGAAATAGCAAGTT | GTCAAGTTCGGCTCC | CTTGAATG | TCTCTCT | TTGACGCGGTTCT | TTTTTT |
| pegRNA-1 | ACCATTCA | AAAATAAGGCTAGTCCGTTATCAAC | GTAGGACAAGATTTG | GTTC | C | ATCTAGTTACGCG | |
| | AGAGC | TTGAAAAAGTGGCACCGAGTCGGT | ATTAGCGAACCTATC | | | TTAAACCAACTAG | |
| | | GC | AAGTT | | | AAA | |
| PAH, exon 4 recode-twinPE- | GCTACGGG | GTTTTAGAGCTAGAAATAGCAAGTT | GCTAATCAAATCTTG | GAGTCCAT | TCTCTCT | TTGACGCGGTTCT | TTTTTT |
| pegRNA-2 | CCATGGAC | AAAATAAGGCTAGTCCGTTATCAAC | TCCTACGGAGCCGAA | GGCC | C | ATCTAGTTACGCG | |
| | TCACA | TTGAAAAAGTGGCACCGAGTCGGT | CTTGACGCTGATCAT | | | TTAAACCAACTAG | |
| | | GC | CCTGT | | | AAA | |
| Rosa26, attB insertion- | GTCTACTGT | GTTTTAGAGCTAGAAATAGCAAGTT | ATGATCCTGACGACG | TTAGAGTG | CAGTAG | CGCGGTTCTATCTA | TTTTTT |
| twinPE-pegRNA1 | TCACTCTA | AAAATAAGGCTAGTCCGTTATCAAC | GAGACCGCCGTCGTC | AA | | GTTACGCGTTAAA | |
| | ACAG | TTGAAAAAGTGGCACCGAGTCGGT | GACAAGCC | | | CCAACTAGAA | |
| | | GC | | | | | |
| Rosa26, attB insertion- | GTAATCTG | GTTTTAGAGCTAGAAATAGCAAGTT | GGCTTGTCGACGACG | GATATACT | CAGATT | CGCGGTTCTATCTA | TTTTTT |
| twinPE-pegRNA2 | CTAGTATA | AAAATAAGGCTAGTCCGTTATCAAC | GCGGTCTCCGTCGTC | | | GTTACGCGTTAAA | |
| | TCCGT | TTGAAAAAGTGGCACCGAGTCGGT | AGGATCAT | AG | | CCAACTAGAA | |
| | | GC | | | | | |
| mDnmt1, loxP insertion | GCGGGCTG | GTTTTAGAGCTAGAAATAGCAAGTT | GAGCTGTTCTGTCGT | CGAACAGC | | CGCGGTTCTATCTA | TTTTTT |
| pegRNA, tevoPreQ1 | GAGCTGTT | AAAATAAGGCTAGTCCGTTATCAAC | CTGCAACCTGCAAGA | T | | GTTACGCGTTAAA | |
| | CGCGC | TTGAAAAAGTGGCACCGAGTCGGT | TGCCAGCGATTGTTA | | | CCAACTAGAA | |
| | | GC | TAACTTCGTATAGCA | | | | |
| | | | TACATTATACGAAGT | | | | |
| | | | TATCG | | | | |
| mDnmt1, nicking sgRNA | GCCGCGCG | GTTTTAGAGCTAGAAATAGCAAGTT | | | | | TTTTTT |
| | CGCGAAAA | AAAATAAGGCTAGTCCGTTATCAAC | | | | | |
| | AGCCG | TTGAAAAAGTGGCACCGAGTCGGT | | | | | |
| | | GC | | | | | |
| HEK3, +5 G to T | GGCCCAGA | GTTTTAGAGCTAGAAATAGCAAGTT | TCTGCAATCA | CGTGCTCA | | | TTTTTT |
| | CTGAGCAC | AAAATAAGGCTAGTCCGTTATCAAC | | GTCTG | | | |
| | GTGA | TTGAAAAAGTGGGACCGAGTCGGT | | | | | |
| | | CC | | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| HEK3, + 1 FLAG ins | GGCCCAGA | GTTTTAGAGCTAGAAATAGCAAGTT | TGGAGGAAGCAGGG | CGTGCTCA | TCTCTCT | GGGTCAGGAGCCC | TTTTTT |
| (mpknot) | CTGAGCAC | AAAATAAGGCTAGTCCGTTATCAAC | CTTCCTTTCCTCTGCC | GTCTG | C | CCCCCCTGAACCC | |
| | GTGA | TTGAAAAAGTGGCACCGAGTCGGT | ATCACTTATCGTCGT | | | AGGATAACCCTCA | |
| | | GC | CATCCTTGTAATC | | | AAGTCGGGGGGCA | |
| | | | | | | ACCC | |
| DNMT1, 1-15 deletion | GATTCCTG | GTTTTAGAGCTAGAAATAGCAAGTT | AGGAGGAAGCTGCT | TTCTGGCA | CCTCTTC | TTGACGCGGTTCT | TTTTTT |
| (evopreQ1) | GTGCCAGA | AAAATAAGGCTAGTCCGTTATCAAC | AAGGACTAGTTCTGC | CCAGGA | T | ATCTAGTTACGCG | |
| | AACA | TTGAAAAAGTGGCACCGAGTCGGT | CC | | | TTAAACCAACTAG | |
| | | GC | | | | AAA | |
| RNF2, 1-15 deletion | GTCATCTTA | GTTTTAGAGCTAGAAATAGCAAGTT | TAAGCAAAACATGG | GTAATGAC | TCATCTC | TTGACGCGGTTCT | TTTTTT |
| (evopreQ1) | GTCATTAC | AAAATAAGGCTAGTCCGTTATCAAC | GAACTCAGTTTATAT | TAAGATG | T | ATCTAGTTACGCG | |
| | CTG | TTGAAAAAGTGGCACCGAGTCGGT | GAGTTA | | | TTAAACCAACTAG | |
| | | GC | | | | AAA | |
| EMX1, +5 G to T | GAGTCCGA | GTTTTAGAGCTAGAAATAGCAAGTT | ATGGGAGCACTTC | TTCTTCTGC | | | TTTTTT |
| | GCAGAAGA | AAAATAAGGCTAGTCCGTTATCAAC | | TCGG | | | |
| | AGAA | TTGAAAAAGTGGCACCGAGTCGGT | | | | | |
| | | GC | | | | | |
| FANCF, +5 G to T | GGAATCCC | GTTTTAGAGCTAGAAATAGCAAGTT | GGAAAAGCGATCAA | GCTGCAGA | | | TTTTTT |
| | TTCTGCAG | AAAATAAGGCTAGTCCGTTATCAAC | GGT | A | | | |
| | CACC | TTGAAAAAGTGGCACCGAGTCGGT | | | | | |
| | | GC | | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| EMX1, nicking sgRNA | GCCGTTTGT | GTTTTAGAGCTAGAAATAGCAAGTT | | | | | TTTTTT |
| | ACTTTGTCC | AAAATAAGGCTAGTCCGTTATCAAC | | | | | |
| | TC | TTGAAAAAGTGGCACCGAGTCGGT | | | | | |
| | | GC | | | | | |
| DNMT1, 1-15 deletion | GATTCCTG | GTTTTAGAGCTAGAAATAGCAAGTT | AGGAGGAAGCTGCT | TTCTGGCA | CCTCTTC | TTGACGCGGTTCT | TTTTTT |
| (evopreQ1) | GTGCCAGA | AAAATAAGGCTAGTCCGTTATCAAC | AAGGACTAGTTCTGC | CCAGGA | T | ATCTAGTTACGCG | |
| | AACA | TTGAAAAAGTGGCACCGAGTCGGT | CC | | | TTAAACCAACTAG | |
| | | GC | | | | AAA | |
| RNF2, 1-15 deletion | GTCATCTTA | GTTTTAGAGCTAGAAATAGCAAGTT | TAAGCAAAACATGG | GTAATGAC | TCATCTC | TTGACGCGGTTCT | TTTTTT |
| (evopreQ1) | GTCATTAC | AAAATAAGGCTAGTCCGTTATCAAC | GAACTCAGTTTATAT | TAAGATG | T | ATCTAGTTACGCG | |
| | CTG | TTGAAAAAGTGGCACCGAGTCGGT | GAGTTA | | | TTAAACCAACTAG | |
| | | GC | | | | AAA | |
| DNMT1, +5 G to T | GATTCCTG | GTTTTAGAGCTAGAAATAGCAAGTT | GTCACCACTGT | TTCTGGCA | | | TTTTTT |
| | GTGCCAGA | AAAATAAGGCTAGTCCGTTATCAAC | | CCAGG | | | |
| | AACA | TTGAAAAAGTGGCACCGAGTCGGT | | | | | |
| | | GC | | | | | |
| EMX1, +5 G to T | GAGTCCGA | GTTTTAGAGCTAGAAATAGCAAGTT | ATGGGAGCACTTC | TTCTTCTGC | | | TTTTTT |
| | GCAGAAGA | AAAATAAGGCTAGTCCGTTATCAAC | | TCGG | | | |
| | AGAA | TTGAAAAAGTGGCACCGAGTCGGT | | | | | |
| | | GC | | | | | |
| FANCF, +5 G to T | GGAATCCC | GTTTTAGAGCTAGAAATAGCAAGTT | GGAAAAGCGATCAA | GCTGCAGA | | | TTTTTT |
| | TTCTGCAG | AAAATAAGGCTAGTCCGTTATCAAC | GGT | A | | | |
| | CACC | TTGAAAAAGTGGCACCGAGTCGGT | | | | | |
| | | GC | | | | | |
| HEK3, +5 G to T | GGCCCAGA | GTTTTAGAGCTAGAAATAGCAAGTT | TCTGCAATCA | CGTGCTCA | | | TTTTTT |
| | CTGAGCAC | AAAATAAGGCTAGTCCGTTATCAAC | | GTCTG | | | |
| | GTGA | TTGAAAAAGTGGGACCGAGTCGGT | | | | | |
| | | CC | | | | | |
| RNF2, +5 G to T | GTCATCTTA | GTTTTAGAGCTAGAAATAGCAAGTT | AACGAACACATCAG | GTAATGAC | | | TTTTTT |
| | GTCATTAC | AAAATAAGGCTAGTCCGTTATCAAC | | TAAGATG | | | |
| | CTG | TTGAAAAAGTGGCACCGAGTCGGT | | | | | |
| | | GC | | | | | |
| HEK3, + 1 His ins | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCAATGATGGTGAT | | | | |
| | | GC | GATGGTG | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| DNMT1, nicking sgRNA | GCCCTTCA | GTTTTAGAGCTAGAAATAGCAAGTT | | | | | TTTTTT |
| | GCTAAAAT | AAAATAAGGCTAGTCCGTTATCAAC | | | | | |
| | AAAGG | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| EMX1, nicking sgRNA | GCCGTTTGT | GTTTTAGAGCTAGAAATAGCAAGTT | | | | | TTTTTT |
| | ACTTTGTCC | AAAATAAGGCTAGTCCGTTATCAAC | | | | | |
| | TC | TTGAAAAAGTGGCACCGAGTCGGT | | | | | |
| | | GC | | | | | |
| HEK3, +5 G to T | GGCCCAGA | GTTTTAGAGCTAGAAATAGCAAGTT | TCTGCAATCA | CGTGCTCA | | | TTTTTT |
| | CTGAGCAC | AAAATAAGGCTAGTCCGTTATCAAC | | GTCTG | | | |
| | GTGA | TTGAAAAAGTGGGACCGAGTCGGT | | | | | |
| | | CC | | | | | |
| AAVS1, attP insertion | GACGTCAC | GTTTTAGAGCTAGAAATAGCAAGTT | TACCGTACACCACTG | GGCAGCGC | | | TTTTTT |
| twinPE-pegRNA1 | GGCGCTGC | AAAATAAGGCTAGTCCGTTATCAAC | AGACCGCGGTGGTTG | C | | | |
| | CCCA | TTGAAAAAGTGGCACCGAGTCGGT | ACCAGACAAACCT | | | | |
| | | GC | | | | | |
| AAVS1, attP insertion | GGACTTCC | GTTTTAGAGCTAGAAATAGCAAGTT | GTCTGGTCAACCACC | TGCACACT | | | TTTTTT |
| twinPE-pegRNA2 | CAGTGTGC | AAAATAAGGCTAGTCCGTTATCAAC | GCGGTCTCAGTGGTG | G | | | |
| | ATCG | TTGAAAAAGTGGCACCGAGTCGGT | TACGGTACAAACCT | | | | |
| | | GC | | | | | |
| DNMT1, 1-15 deletion | GATTCCTG | GTTTTAGAGCTAGAAATAGCAAGTT | AGGAGGAAGCTGCT | TTCTGGCA | CCTCTTC | TTGACGCGGTTCT | TTTTTT |
| (evopreQ1) | GTGCCAGA | AAAATAAGGCTAGTCCGTTATCAAC | AAGGACTAGTTCTGC | CCAGGA | T | ATCTAGTTACGCG | |
| | AACA | TTGAAAAAGTGGCACCGAGTCGGT | CC | | | TTAAACCAACTAG | |
| | | GC | | | | AAA | |
| RNF2, 1-15 deletion | GTCATCTTA | GTTTTAGAGCTAGAAATAGCAAGTT | TAAGCAAAACATGG | GTAATGAC | TCATCTC | TTGACGCGGTTCT | TTTTTT |
| (evopreQ1) | GTCATTAC | AAAATAAGGCTAGTCCGTTATCAAC | GAACTCAGTTTATAT | TAAGATG | T | ATCTAGTTACGCG | |
| | CTG | TTGAAAAAGTGGCACCGAGTCGGT | GAGTTA | | | TTAAACCAACTAG | |
| | | GC | | | | AAA | |
| FANCF, +5 G to T | GGAATCCC | GTTTTAGAGCTAGAAATAGCAAGTT | GGAAAAGCGATCAA | GCTGCAGA | | | TTTTTT |
| | TTCTGCAG | AAAATAAGGCTAGTCCGTTATCAAC | GGT | A | | | |
| | CACC | TTGAAAAAGTGGCACCGAGTCGGT | | | | | |
| | | GC | | | | | |
| PRNP, +6 G to T | GCAGTGGT | GTTTTAGAGCTAGAAATAGCAAGTT | ATGTAGACGCCA | AGGCCCCC | TAGACAC | CGCGGTTCTATCTA | TTTTTT |
| | GGGGGGCC | AAAATAAGGCTAGTCCGTTATCAAC | | CACC | A | GTTACGCGTTAAA | |
| | TTGG | TTGAAAAAGTGGCACCGAGTCGGT | | | | CCAACTAGAA | |
| | | GC | | | | | |
| HEK3, +1 T to A | GGCCCAGA | GTTTAAGAGCTATGCTGGAAACAGC | TTCCTCTGCCATCT | CGTGCTCA | TCTCTCT | GGGTCAGGAGCCC | TTTTTT |
| | CTGAGCAC | ATAGCAAGTTTAAATAAGGCTAGTC | | GTCTG | C | CCCCCCTGAACCC | |
| | GTGA | CGTTATCAACTTGAAAAAGTGGCAC | | | | AGGATAACCCTCA | |
| | | CGAGTCGGTGC | | | | AAGTCGGGGGGCA | |
| | | | | | | ACCC | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| PRNP, nicking sgRNA | GCATGTTTT | GTTTTAGAGCTAGAAATAGCAAGTT | | | | | TTTTTT |
| | CACGATAG | AAAATAAGGCTAGTCCGTTATCAAC | | | | | |
| | TAA | TTGAAAAAGTGGCACCGAGTCGGT | | | | | |
| | | GC | | | | | |
| DNMT1, 1-15 deletion | GATTCCTG | GTTTTAGAGCTAGAAATAGCAAGTT | AGGAGGAAGCTGCT | TTCTGGCA | CCTCTTC | TTGACGCGGTTCT | TTTTTT |
| (evopreQ1) | GTGCCAGA | AAAATAAGGCTAGTCCGTTATCAAC | AAGGACTAGTTCTGC | CCAGGA | T | ATCTAGTTACGCG | |
| | AACA | TTGAAAAAGTGGCACCGAGTCGGT | CC | | | TTAAACCAACTAG | |
| | | GC | | | | AAA | |
| RNF2, 1-15 deletion | GTCATCTTA | GTTTTAGAGCTAGAAATAGCAAGTT | TAAGCAAAACATGG | GTAATGAC | TCATCTC | TTGACGCGGTTCT | TTTTTT |
| (evopreQ1) | GTCATTAC | AAAATAAGGCTAGTCCGTTATCAAC | GAACTCAGTTTATAT | TAAGATG | T | ATCTAGTTACGCG | |
| | CTG | TTGAAAAAGTGGCACCGAGTCGGT | GAGTTA | | | TTAAACCAACTAG | |
| | | GC | | | | AAA | |
| HEK3, +5 G to T | GGCCCAGA | GTTTTAGAGCTAGAAATAGCAAGTT | TCTGCAATCA | CGTGCTCA | | | TTTTTT |
| | CTGAGCAC | AAAATAAGGCTAGTCCGTTATCAAC | | GTCTG | | | |
| | GTGA | TTGAAAAAGTGGGACCGAGTCGGT | | | | | |
| | | CC | | | | | |
| PRNP, +6 G to T | GCAGTGGT | GTTTTAGAGCTAGAAATAGCAAGTT | ATGTAGACGCCA | AGGCCCCC | TAGACAC | CGCGGTTCTATCTA | TTTTTT |
| | GGGGGGCC | AAAATAAGGCTAGTCCGTTATCAAC | | CACC | A | GTTACGCGTTAAA | |
| | TTGG | TTGAAAAAGTGGCACCGAGTCGGT | | | | CCAACTAGAA | |
| | | GC | | | | | |
| HEK3, + 1 FLAG ins | GGCCCAGA | GTTTTAGAGCTAGAAATAGCAAGTT | TGGAGGAAGCAGGG | CGTGCTCA | TCTCTCT | GGGTCAGGAGCCC | TTTTTT |
| (mpknot) | CTGAGCAC | AAAATAAGGCTAGTCCGTTATCAAC | CTTCCTTTCCTCTGCC | GTCTG | C | CCCCCCTGAACCC | |
| | GTGA | TTGAAAAAGTGGCACCGAGTCGGT | ATCACTTATCGTCGT | | | AGGATAACCCTCA | |
| | | GC | CATCCTTGTAATC | | | AAGTCGGGGGGCA | |
| | | | | | | ACCC | |
| RNF2, +5 G to T | GTCATCTTA | GTTTTAGAGCTAGAAATAGCAAGTT | AACGAACACATCAG | GTAATGAC | | | TTTTTT |
| | GTCATTAC | AAAATAAGGCTAGTCCGTTATCAAC | | TAAGATG | | | |
| | CTG | TTGAAAAAGTGGCACCGAGTCGGT | | | | | |
| | | GC | | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| DNMT1, nicking sgRNA | GCCCTTCA | GTTTTAGAGCTAGAAATAGCAAGTT | | | | | TTTTTT |
| | GCTAAAAT | AAAATAAGGCTAGTCCGTTATCAAC | | | | | |
| | AAAGG | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| PRNP, nicking sgRNA | GCATGTTTT | GTTTTAGAGCTAGAAATAGCAAGTT | | | | | TTTTTT |
| | CACGATAG | AAAATAAGGCTAGTCCGTTATCAAC | | | | | |
| | TAA | TTGAAAAAGTGGCACCGAGTCGGT | | | | | |
| | | GC | | | | | |
| HEK3, + 1 FLAG ins | GGCCCAGA | GTTTTAGAGCTAGAAATAGCAAGTT | TGGAGGAAGCAGGG | CGTGCTCA | TCTCTCT | GGGTCAGGAGCCC | TTTTTT |
| (mpknot) | CTGAGCAC | AAAATAAGGCTAGTCCGTTATCAAC | CTTCCTTTCCTCTGCC | GTCTG | C | CCCCCCTGAACCC | |
| | GTGA | TTGAAAAAGTGGCACCGAGTCGGT | ATCACTTATCGTCGT | | | AGGATAACCCTCA | |
| | | GC | CATCCTTGTAATC | | | AAGTCGGGGGGCA | |
| | | | | | | ACCC | |
| DNMT1, 1-15 deletion | GATTCCTG | GTTTTAGAGCTAGAAATAGCAAGTT | AGGAGGAAGCTGCT | TTCTGGCA | CCTCTTC | TTGACGCGGTTCT | TTTTTT |
| (evopreQ1) | GTGCCAGA | AAAATAAGGCTAGTCCGTTATCAAC | AAGGACTAGTTCTGC | CCAGGA | T | ATCTAGTTACGCG | |
| | AACA | TTGAAAAAGTGGCACCGAGTCGGT | CC | | | TTAAACCAACTAG | |
| | | GC | | | | AAA | |
| RNF2, 1-15 deletion | GTCATCTTA | GTTTTAGAGCTAGAAATAGCAAGTT | TAAGCAAAACATGG | GTAATGAC | TCATCTC | TTGACGCGGTTCT | TTTTTT |
| (evopreQ1) | GTCATTAC | AAAATAAGGCTAGTCCGTTATCAAC | GAACTCAGTTTATAT | TAAGATG | T | ATCTAGTTACGCG | |
| | CTG | TTGAAAAAGTGGCACCGAGTCGGT | GAGTTA | | | TTAAACCAACTAG | |
| | | GC | | | | AAA | |
| EMX1, +5 G to T | GAGTCCGA | GTTTTAGAGCTAGAAATAGCAAGTT | ATGGGAGCACTTC | TTCTTCTGC | | | TTTTTT |
| | GCAGAAGA | AAAATAAGGCTAGTCCGTTATCAAC | | TCGG | | | |
| | AGAA | TTGAAAAAGTGGCACCGAGTCGGT | | | | | |
| | | GC | | | | | |
| FANCF, +5 G to T | GGAATCCC | GTTTTAGAGCTAGAAATAGCAAGTT | GGAAAAGCGATCAA | GCTGCAGA | | | TTTTTT |
| | TTCTGCAG | AAAATAAGGCTAGTCCGTTATCAAC | GGT | A | | | |
| | CACC | TTGAAAAAGTGGCACCGAGTCGGT | | | | | |
| | | GC | | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| EMX1, nicking sgRNA | GCCGTTTGT | GTTTTAGAGCTAGAAATAGCAAGTT | | | | | TTTTTT |
| | ACTTTGTCC | AAAATAAGGCTAGTCCGTTATCAAC | | | | | |
| | TC | TTGAAAAAGTGGCACCGAGTCGGT | | | | | |
| | | GC | | | | | |
| Correction of 20-bp deletion | GCAATGAA | GTTTTAGAGCTAGAAATAGCAAGTT | TCCAGTCTTCTTATCT | TTAAGCCA | | | TTTTTT |
| in T7 RNAP, (v2 circuit) | CTGGCTTA | AAAATAAGGCTAGTCCGTTATCAAC | TTGACCTCAGCAGCC | G | | | |
| | AGTC | TTGAAAAAGTGGCACCGAGTCGGT | AGCAGCTTAGCAGCA | | | | |
| | | GC | GAC | | | | |
| Correction of 20-bp deletion | GCAATGAA | GTTTTAGAGCTAGAAATAGCAAGTT | TCTCTCCAGTCTTCTT | TTAAGCCA | | | TTTTTT |
| in T7 RNAP, (v2 circuit) | CTGGCTTA | AAAATAAGGCTAGTCCGTTATCAAC | ATCTTTGACCTCAGC | G | | | |
| | AGTC | TTGAAAAAGTGGCACCGAGTCGGT | AGCCAGCAGCTTAGC | | | | |
| | | GC | AGCAGAC | | | | |
| Correction of 20-bp deletion | GCAATGAA | GTTTTAGAGCTAGAAATAGCAAGTT | AATCTCTCCAGTCTT | TTAAGCCA | | | TTTTTT |
| in T7 RNAP, (v2 circuit) | CTGGCTTA | AAAATAAGGCTAGTCCGTTATCAAC | CTTATCTTTGACCTC | G | | | |
| | AGTC | TTGAAAAAGTGGCACCGAGTCGGT | AGCAGCCAGCAGCTT | | | | |
| | | GC | AGCAGCAGAC | | | | |
| Correction of 20-bp deletion | GCAATGAA | GTTTTAGAGCTAGAAATAGCAAGTT | GAAGAATCTCTCCAG | TTAAGCCA | | | TTTTTT |
| in T7 RNAP, (v2 circuit) | CTGGCTTA | AAAATAAGGCTAGTCCGTTATCAAC | TCTTCTTATCTTTGAC | G | | | |
| | AGTC | TTGAAAAAGTGGCACCGAGTCGGT | CTCAGCAGCCAGCAG | | | | |
| | | GC | CTTAGCAGCAGAC | | | | |
| Correction of 20-bp deletion | GCAATGAA | GTTTTAGAGCTAGAAATAGCAAGTT | TTGCGAAGAATCTCT | TTAAGCCA | | | TTTTTT |
| in T7 RNAP, (v2 circuit) | CTGGCTTA | AAAATAAGGCTAGTCCGTTATCAAC | CCAGTCTTCTTATCTT | G | | | |
| | AGTC | TTGAAAAAGTGGCACCGAGTCGGT | TGACCTCAGCAGCCA | | | | |
| | | GC | GCAGCTTAGCAGCAG | | | | |
| | | | AC | | | | |
| Correction of 20-bp deletion | GCAATGAA | GTTTTAGAGCTAGAAATAGCAAGTT | GTCTTCTTATCTTTGA | TTAAGCCA | | | TTTTTT |
| in T7 RNAP, (v2 circuit) | CTGGCTTA | AAAATAAGGCTAGTCCGTTATCAAC | CCTCAGCAGCCAGCA | GT | | | |
| | AGTC | TTGAAAAAGTGGCACCGAGTCGGT | GCTTAGCAGCAGAC | | | | |
| | | GC | | | | | |
| Correction of 20-bp deletion | GCAATGAA | GTTTTAGAGCTAGAAATAGCAAGTT | TCCAGTCTTCTTATCT | TTAAGCCA | | | TTTTTT |
| in T7 RNAP, (v2 circuit) | CTGGCTTA | AAAATAAGGCTAGTCCGTTATCAAC | TTGACCTCAGCAGCC | GT | | | |
| | AGTC | TTGAAAAAGTGGCACCGAGTCGGT | AGCAGCTTAGCAGCA | | | | |
| | | GC | GAC | | | | |
| Correction of 20-bp deletion | GCAATGAA | GTTTTAGAGCTAGAAATAGCAAGTT | TCTCTCCAGTCTTCTT | TTAAGCCA | | | TTTTTT |
| in T7 RNAP, (v2 circuit) | CTGGCTTA | AAAATAAGGCTAGTCCGTTATCAAC | ATCTTTGACCTCAGC | GT | | | |
| | AGTC | TTGAAAAAGTGGCACCGAGTCGGT | AGCCAGCAGCTTAGC | | | | |
| | | GC | AGCAGAC | | | | |
| Correction of 20-bp deletion | GCAATGAA | GTTTTAGAGCTAGAAATAGCAAGTT | AATCTCTCCAGTCTT | TTAAGCCA | | | TTTTTT |
| in T7 RNAP, (v2 circuit) | CTGGCTTA | AAAATAAGGCTAGTCCGTTATCAAC | CTTATCTTTGACCTC | GT | | | |
| | AGTC | TTGAAAAAGTGGCACCGAGTCGGT | AGCAGCCAGCAGCTT | | | | |
| | | GC | AGCAGCAGAC | | | | |
| Correction of 20-bp deletion | GCAATGAA | GTTTTAGAGCTAGAAATAGCAAGTT | GAAGAATCTCTCCAG | TTAAGCCA | | | TTTTTT |
| in T7 RNAP, (v2 circuit) | CTGGCTTA | AAAATAAGGCTAGTCCGTTATCAAC | TCTTCTTATCTTTGAC | GT | | | |
| | AGTC | TTGAAAAAGTGGCACCGAGTCGGT | CTCAGCAGCCAGCAG | | | | |
| | | GC | CTTAGCAGCAGAC | | | | |
| Correction of 20-bp deletion | GCAATGAA | GTTTTAGAGCTAGAAATAGCAAGTT | TTGCGAAGAATCTCT | TTAAGCCA | | | TTTTTT |
| in T7 RNAP, (v2 circuit) | CTGGCTTA | AAAATAAGGCTAGTCCGTTATCAAC | CCAGTCTTCTTATCTT | GT | | | |
| | AGTC | TTGAAAAAGTGGCACCGAGTCGGT | TGACCTCAGCAGCCA | | | | |
| | | GC | GCAGCTTAGCAGCAG | | | | |
| | | | AC | | | | |
| Correction of 20-bp deletion | GCAATGAA | GTTTTAGAGCTAGAAATAGCAAGTT | TCTCTCCAGTCTTCTT | TTAAGCCA | | | TTTTTT |
| in T7 RNAP, (v2 circuit) | CTGGCTTA | AAAATAAGGCTAGTCCGTTATCAAC | ATCTTTGACCTCAGC | GTTC | | | |
| | AGTC | TTGAAAAAGTGGCACCGAGTCGGT | AGCCAGCAGCTTAGC | | | | |
| | | GC | AGCAGAC | | | | |
| Correction of 20-bp deletion | GCAATGAA | GTTTTAGAGCTAGAAATAGCAAGTT | AATCTCTCCAGTCTT | TTAAGCCA | | | TTTTTT |
| in T7 RNAP, (v2 circuit) | CTGGCTTA | AAAATAAGGCTAGTCCGTTATCAAC | CTTATCTTTGACCTC | GTTC | | | |
| | AGTC | TTGAAAAAGTGGCACCGAGTCGGT | AGCAGCCAGCAGCTT | | | | |
| | | GC | AGCAGCAGAC | | | | |
| Correction of 20-bp deletion | GCAATGAA | GTTTTAGAGCTAGAAATAGCAAGTT | GAAGAATCTCTCCAG | TTAAGCCA | | | TTTTTT |
| in T7 RNAP, (v2 circuit) | CTGGCTTA | AAAATAAGGCTAGTCCGTTATCAAC | TCTTCTTATCTTTGAC | GTTC | | | |
| | AGTC | TTGAAAAAGTGGCACCGAGTCGGT | CTCAGCAGCCAGCAG | | | | |
| | | GC | CTTAGCAGCAGAC | | | | |
| Correction of 20-bp deletion | GCAATGAA | GTTTTAGAGCTAGAAATAGCAAGTT | TTGCGAAGAATCTCT | TTAAGCCA | | | TTTTTT |
| in T7 RNAP, (v2 circuit) | CTGGCTTA | AAAATAAGGCTAGTCCGTTATCAAC | CCAGTCTTCTTATCTT | GTTC | | | |
| | AGTC | TTGAAAAAGTGGCACCGAGTCGGT | TGACCTCAGCAGCCA | | | | |
| | | GC | GCAGCTTAGCAGCAG | | | | |
| | | | AC | | | | |
| Correction of 20-bp deletion | GCAATGAA | GTTTTAGAGCTAGAAATAGCAAGTT | TCTCTCCAGTCTTCTT | TTAAGCCA | | | TTTTTT |
| in T7 RNAP, (v2 circuit) | CTGGCTTA | AAAATAAGGCTAGTCCGTTATCAAC | ATCTTTGACCTCAGC | GTTCA | | | |
| | AGTC | TTGAAAAAGTGGCACCGAGTCGGT | AGCCAGCAGCTTAGC | | | | |
| | | GC | AGCAGAC | | | | |
| Correction of 20-bp deletion | GCAATGAA | GTTTTAGAGCTAGAAATAGCAAGTT | GAAGAATCTCTCCAG | TTAAGCCA | | | TTTTTT |
| in T7 RNAP, (v2 circuit) | CTGGCTTA | AAAATAAGGCTAGTCCGTTATCAAC | TCTTCTTATCTTTGAC | GTTCA | | | |
| | AGTC | TTGAAAAAGTGGCACCGAGTCGGT | CTCAGCAGCCAGCAG | | | | |
| | | GC | CTTAGCAGCAGAC | | | | |
| Correction of 20-bp deletion | GCAATGAA | GTTTTAGAGCTAGAAATAGCAAGTT | TTGCGAAGAATCTCT | TTAAGCCA | | | TTTTTT |
| in T7 RNAP, (v2 circuit) | CTGGCTTA | AAAATAAGGCTAGTCCGTTATCAAC | CCAGTCTTCTTATCTT | GTTCA | | | |
| | AGTC | TTGAAAAAGTGGCACCGAGTCGGT | TGACCTCAGCAGCCA | | | | |
| | | GC | GCAGCTTAGCAGCAG | | | | |
| | | | AC | | | | |
| Correction of 20-bp deletion | GCAATGAA | GTTTTAGAGCTAGAAATAGCAAGTT | GTCTTCTTATCTTTGA | TTAAGCCA | | | TTTTTT |
| in T7 RNAP, (v2 circuit) | CTGGCTTA | AAAATAAGGCTAGTCCGTTATCAAC | CCTCAGCAGCCAGCA | GTTCAT | | | |
| | AGTC | TTGAAAAAGTGGCACCGAGTCGGT | GCTTAGCAGCAGAC | | | | |
| | | GC | | | | | |
| Correction of 20-bp deletion | GCAATGAA | GTTTTAGAGCTAGAAATAGCAAGTT | TCCAGTCTTCTTATCT | TTAAGCCA | | | TTTTTT |
| in T7 RNAP, (v2 circuit) | CTGGCTTA | AAAATAAGGCTAGTCCGTTATCAAC | TTGACCTCAGCAGCC | GTTCAT | | | |
| | AGTC | TTGAAAAAGTGGCACCGAGTCGGT | AGCAGCTTAGCAGCA | | | | |
| | | GC | GAC | | | | |
| Correction of 20-bp deletion | GCAATGAA | GTTTTAGAGCTAGAAATAGCAAGTT | AATCTCTCCAGTCTT | TTAAGCCA | | | TTTTTT |
| in T7 RNAP, (v2 circuit) | CTGGCTTA | AAAATAAGGCTAGTCCGTTATCAAC | CTTATCTTTGACCTC | GTTCAT | | | |
| | AGTC | TTGAAAAAGTGGCACCGAGTCGGT | AGCAGCCAGCAGCTT | | | | |
| | | GC | AGCAGCAGAC | | | | |
| Correction of 20-bp deletion | GCAATGAA | GTTTTAGAGCTAGAAATAGCAAGTT | GAAGAATCTCTCCAG | TTAAGCCA | | | TTTTTT |
| in T7 RNAP, (v2 circuit) | CTGGCTTA | AAAATAAGGCTAGTCCGTTATCAAC | TCTTCTTATCTTTGAC | GTTCAT | | | |
| | AGTC | TTGAAAAAGTGGCACCGAGTCGGT | CTCAGCAGCCAGCAG | | | | |
| | | GC | CTTAGCAGCAGAC | | | | |
| Correction of 20-bp deletion | GCAATGAA | GTTTTAGAGCTAGAAATAGCAAGTT | TTGCGAAGAATCTCT | TTAAGCCA | | | TTTTTT |
| in T7 RNAP, (v2 circuit) | CTGGCTTA | AAAATAAGGCTAGTCCGTTATCAAC | CCAGTCTTCTTATCTT | GTTCAT | | | |
| | AGTC | TTGAAAAAGTGGCACCGAGTCGGT | TGACCTCAGCAGCCA | | | | |
| | | GC | GCAGCTTAGCAGCAG | | | | |
| | | | AC | | | | |
| Correction of 1-bp deletion in | GCTCGCGA | GTTTTAGAGCTAGAAATAGCAAGTT | AGACTCATGCTCAAG | GCCAACTG | | | TTTTTT |
| T7 RNAP, (+6 ins A, v1 | ACAGTTGG | AAAATAAGGCTAGTCCGTTATCAAC | G | T | | | |
| circuit) | CCCT | TTGAAAAAGTGGCACCGAGTCGGT | | | | | |
| | | GC | | | | | |
| Correction of 20-bp deletion | GCAATGAA | GTTTTAGAGCTAGAAATAGCAAGTT | TTGCGAAGAATCTCT | TTAAGCCA | | | TTTTTT |
| in T7 RNAP, (v2 circuit) | CTGGCTTA | AAAATAAGGCTAGTCCGTTATCAAC | CCAGTCTTCTTATCTT | G | | | |
| | AGTC | TTGAAAAAGTGGCACCGAGTCGGT | TGACCTCAGCAGCCA | | | | |
| | | GC | GCAGCTTAGCAGCAG | | | | |
| | | | AC | | | | |
| Correction of 20-bp deletion | TTGACGGA | GTTTTAGAGCTAGAAATAGCAAGTT | ACGGTGTTACTCGCA | AGTTCGGC | | CGCGGTTCTATCTA | TTTTTT |
| in T7 RNAP, (v3 circuit) | AGCCGAAC | AAAATAAGGCTAGTCCGTTATCAAC | GTGTGACTAAGCGTT | T | | GTTACGCGTTAAA | |
| | TCTT | TTGAAAAAGTGGCACCGAGTCGGT | CAGTCATGACGCTGG | | | CCAACTAGAA | |
| | | GC | CTTACGGGAGTAAGG | | | | |
| HEK3, + 1 FLAG ins | GGCCCAGA | GTTTTAGAGCTAGAAATAGCAAGTT | TGGAGGAAGCAGGG | CGTGCTCA | TCTCTCT | GGGTCAGGAGCCC | TTTTTT |
| (mpknot) | CTGAGCAC | AAAATAAGGCTAGTCCGTTATCAAC | CTTCCTTTCCTCTGCC | GTCTG | C | CCCCCCTGAACCC | |
| | GTGA | TTGAAAAAGTGGCACCGAGTCGGT | ATCACTTATCGTCGT | | | AGGATAACCCTCA | |
| | | GC | CATCCTTGTAATC | | | AAGTCGGGGGGCA | |
| | | | | | | ACCC | |
| DNMT1, 1-15 deletion | GATTCCTG | GTTTTAGAGCTAGAAATAGCAAGTT | AGGAGGAAGCTGCT | TTCTGGCA | CCTCTTC | TTGACGCGGTTCT | TTTTTT |
| (evopreQ1) | GTGCCAGA | AAAATAAGGCTAGTCCGTTATCAAC | AAGGACTAGTTCTGC | CCAGGA | T | ATCTAGTTACGCG | |
| | AACA | TTGAAAAAGTGGCACCGAGTCGGT | CC | | | TTAAACCAACTAG | |
| | | GC | | | | AAA | |
| RNF2, 1-15 deletion | GTCATCTTA | GTTTTAGAGCTAGAAATAGCAAGTT | TAAGCAAAACATGG | GTAATGAC | TCATCTC | TTGACGCGGTTCT | TTTTTT |
| (evopreQ1) | GTCATTAC | AAAATAAGGCTAGTCCGTTATCAAC | GAACTCAGTTTATAT | TAAGATG | T | ATCTAGTTACGCG | |
| | CTG | TTGAAAAAGTGGCACCGAGTCGGT | GAGTTA | | | TTAAACCAACTAG | |
| | | GC | | | | AAA | |
| EMX1, 1-15 deletion | GAGTCCGA | GTTTTAGAGCTAGAAATAGCAAGTT | TTGGCCTGCTTCGTG | TTCTTCTGC | AACAATC | TTGACGCGGTTCT | TTTTTT |
| (evopreQ1) | GCAGAAGA | AAAATAAGGCTAGTCCGTTATCAAC | GCAATGCGCCACCGG | TCGGA | T | ATCTAGTTACGCG | |
| | AGAA | TTGAAAAAGTGGCACCGAGTCGGT | TTGATG | | | TTAAACCAACTAG | |
| | | GC | | | | AAA | |
| EMX1, +5 G to T (evopreQ1) | GAGTCCGA | GTTTTAGAGCTAGAAATAGCAAGTT | GTGATGGGAGCACTT | TTCTTCTGC | AACAATC | TTGACGCGGTTCT | TTTTTT |
| | GCAGAAGA | AAAATAAGGCTAGTCCGTTATCAAC | C | TCGGA | T | ATCTAGTTACGCG | |
| | AGAA | TTGAAAAAGTGGCACCGAGTCGGT | | | | TTAAACCAACTAG | |
| | | GC | | | | AAA | |
| RUNX1, +5 G to T | GCATTTTCA | GTTTTAGAGCTAGAAATAGCAAGTT | AGGAAATGACTCAA | CTTCCTCCT | AACTCTC | TTGACGCGGTTCT | TTTTTT |
| (evopreQ1) | GGAGGAAG | AAAATAAGGCTAGTCCGTTATCAAC | ATATGCTGTCTGAAG | GAAAAT | T | ATCTAGTTACGCG | |
| | CGA | TTGAAAAAGTGGCACCGAGTCGGT | CAATCG | | | TTAAACCAACTAG | |
| | | GC | | | | AAA | |
| HEK3, + 1 FLAG ins (no | GGCCCAGA | GTTTTAGAGCTAGAAATAGCAAGTT | TGGAGGAAGCAGGG | CGTGCTCA | | | TTTTTT |
| motif) | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCACTTATCGTCGT | | | | |
| | | GC | CATCCTTGTAATC | | | | |
| DNMT1, 1-15 deletion (no | GATTCCTG | GTTTTAGAGCTAGAAATAGCAAGTT | AGGAGGAAGCTGCT | TTCTGGCA | | | TTTTTT |
| motif) | GTGCCAGA | AAAATAAGGCTAGTCCGTTATCAAC | AAGGACTAGTTCTGC | CCAGGA | | | |
| | AACA | TTGAAAAAGTGGCACCGAGTCGGT | CC | | | | |
| | | GC | | | | | |
| RNF2, 1-15 deletion (no | GTCATCTTA | GTTTTAGAGCTAGAAATAGCAAGTT | TAAGCAAAACATGG | GTAATGAC | | | TTTTTT |
| motif) | GTCATTAC | AAAATAAGGCTAGTCCGTTATCAAC | GAACTCAGTTTATAT | TAAGATG | | | |
| | CTG | TTGAAAAAGTGGCACCGAGTCGGT | GAGTTA | | | | |
| | | GC | | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| DNMT1, nicking sgRNA | GCCCTTCA | GTTTTAGAGCTAGAAATAGCAAGTT | | | | | TTTTTT |
| | GCTAAAAT | AAAATAAGGCTAGTCCGTTATCAAC | | | | | |
| | AAAGG | TTGAAAAAGTGGCACCGAGTCGGT | | | | | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| EMX1, nicking sgRNA | GCCGTTTGT | GTTTTAGAGCTAGAAATAGCAAGTT | | | | | TTTTTT |
| | ACTTTGTCC | AAAATAAGGCTAGTCCGTTATCAAC | | | | | |
| | TC | TTGAAAAAGTGGCACCGAGTCGGT | | | | | |
| | | GC | | | | | |
| RUNX1, nicking sgRNA | GATGAAGC | GTTTTAGAGCTAGAAATAGCAAGTT | | | | | TTTTTT |
| | ACTGTGGG | AAAATAAGGCTAGTCCGTTATCAAC | | | | | |
| | TACGA | TTGAAAAAGTGGCACCGAGTCGGT | | | | | |
| | | GC | | | | | |
| RNF2, +5 G to T | GTCATCTTA | GTTTTAGAGCTAGAAATAGCAAGTT | AACGAACACATCAG | GTAATGAC | | | TTTTTT |
| | GTCATTAC | AAAATAAGGCTAGTCCGTTATCAAC | | TAAGATG | | | |
| | CTG | TTGAAAAAGTGGCACCGAGTCGGT | | | | | |
| | | GC | | | | | |
| HEK3, + 1 FLAG ins | GGCCCAGA | GTTTTAGAGCTAGAAATAGCAAGTT | TGGAGGAAGCAGGG | CGTGCTCA | TCTCTCT | GGGTCAGGAGCCC | TTTTTT |
| (mpknot) | CTGAGCAC | AAAATAAGGCTAGTCCGTTATCAAC | CTTCCTTTCCTCTGCC | GTCTG | C | CCCCCCTGAACCC | |
| | GTGA | TTGAAAAAGTGGCACCGAGTCGGT | ATCACTTATCGTCGT | | | AGGATAACCCTCA | |
| | | GC | CATCCTTGTAATC | | | AAGTCGGGGGGCA | |
| | | | | | | ACCC | |
| EMX1, +5 G to T | GAGTCCGA | GTTTTAGAGCTAGAAATAGCAAGTT | ATGGGAGCACTTC | TTCTTCTGC | | | TTTTTT |
| | GCAGAAGA | AAAATAAGGCTAGTCCGTTATCAAC | | TCGG | | | |
| | AGAA | TTGAAAAAGTGGCACCGAGTCGGT | | | | | |
| | | GC | | | | | |
| HEK3, +1 loxP ins | GGCCCAGA | GTTTTAGAGCTAGAAATAGCAAGTT | TGGAGGAAGCAGGG | CGTGCTCA | | | TTTTTT |
| | CTGAGCAC | AAAATAAGGCTAGTCCGTTATCAAC | CTTCCTTTCCTCTGCC | GTCTG | | | |
| | GTGA | TTGAAAAAGTGGCACCGAGTCGGT | ATCAATAACTTCGTA | | | | |
| | | GC | TAATGTATGCTATAC | | | | |
| | | | GAAGTTATAACAAT | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| HEK3, nicking sgRNA | GTCAACCA | GTTTTAGAGCTAGAAATAGCAAGTT | | | | | TTTTTT |
| | GTATCCCG | AAAATAAGGCTAGTCCGTTATCAAC | | | | | |
| | GTGC | TTGAAAAAGTGGCACCGAGTCGGT | | | | | |
| | | GC | | | | | |
| EMX1, nicking sgRNA | GCCGTTTGT | GTTTTAGAGCTAGAAATAGCAAGTT | | | | | TTTTTT |
| | ACTTTGTCC | AAAATAAGGCTAGTCCGTTATCAAC | | | | | |
| | TC | TTGAAAAAGTGGCACCGAGTCGGT | | | | | |
| | | GC | | | | | |
| HEK3, + 1 FLAG ins | GGCCCAGA | GTTTTAGAGCTAGAAATAGCAAGTT | TGGAGGAAGCAGGG | CGTGCTCA | TCTCTCT | GGGTCAGGAGCCC | TTTTTT |
| (mpknot) | CTGAGCAC | AAAATAAGGCTAGTCCGTTATCAAC | CTTCCTTTCCTCTGCC | GTCTG | C | CCCCCCTGAACCC | |
| | GTGA | TTGAAAAAGTGGCACCGAGTCGGT | ATCACTTATCGTCGT | | | AGGATAACCCTCA | |
| | | GC | CATCCTTGTAATC | | | AAGTCGGGGGGCA | |
| | | | | | | ACCC | |
| (See pegRNAs for FIG. 4G) | | | | | | | |
| EMX1, +5 G to T (evopreQ1) | GAGTCCGA | GTTTTAGAGCTAGAAATAGCAAGTT | GTGATGGGAGCACTT | TTCTTCTGC | AACAATC | TTGACGCGGTTCT | TTTTTT |
| | GCAGAAGA | AAAATAAGGCTAGTCCGTTATCAAC | C | TCGGA | T | ATCTAGTTACGCG | |
| | AGAA | TTGAAAAAGTGGCACCGAGTCGGT | | | | TTAAACCAACTAG | |
| | | GC | | | | AAA | |
| RUNX1, +5 G to T | GCATTTTCA | GTTTTAGAGCTAGAAATAGCAAGTT | AGGAAATGACTCAA | CTTCCTCCT | AACTCTC | TTGACGCGGTTCT | TTTTTT |
| (evopreQ1) | GGAGGAAG | AAAATAAGGCTAGTCCGTTATCAAC | ATATGCTGTCTGAAG | GAAAAT | T | ATCTAGTTACGCG | |
| | CGA | TTGAAAAAGTGGCACCGAGTCGGT | CAATCG | | | TTAAACCAACTAG | |
| | | GC | | | | AAA | |
| RNF2, +5 G to T | GTCATCTTA | GTTTTAGAGCTAGAAATAGCAAGTT | AACGAACACATCAG | GTAATGAC | | | TTTTTT |
| | GTCATTAC | AAAATAAGGCTAGTCCGTTATCAAC | | TAAGATG | | | |
| | CTG | TTGAAAAAGTGGCACCGAGTCGGT | | | | | |
| | | GC | | | | | |
| FANCF, +5 G to T (mpknot) | GGAATCCC | GTTTTAGAGCTAGAAATAGCAAGTT | GGAAAAGCGATCAA | GCTGCAGA | CAATCAC | GGGTCAGGAGCCC | TTTTTT |
| | TTCTGCAG | AAAATAAGGCTAGTCCGTTATCAAC | GGT | AGGGA | T | CCCCCCTGAACCC | |
| | CACC | TTGAAAAAGTGGCACCGAGTCGGT | | | | AGGATAACCCTCA | |
| | | GC | | | | AAGTCGGGGGGCA | |
| | | | | | | ACCC | |
| PRNP, +6 G to T (evopreQ1) | GCAGTGGT | GTTTTAGAGCTAGAAATAGCAAGTT | ATGTAGACGCCA | AGGCCCCC | TAGACAC | CGCGGTTCTATCTA | TTTTTT |
| | GGGGGGCC | AAAATAAGGCTAGTCCGTTATCAAC | | CACC | A | GTTACGCGTTAAA | |
| | TTGG | TTGAAAAAGTGGCACCGAGTCGGT | | | | CCAACTAGAA | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| EMX1, nicking sgRNA | GCCGTTTGT | GTTTTAGAGCTAGAAATAGCAAGTT | | | | | TTTTTT |
| | ACTTTGTCC | AAAATAAGGCTAGTCCGTTATCAAC | | | | | |
| | TC | TTGAAAAAGTGGCACCGAGTCGGT | | | | | |
| | | GC | | | | | |
| RUNX1, nicking sgRNA | GATGAAGC | GTTTTAGAGCTAGAAATAGCAAGTT | | | | | TTTTTT |
| | ACTGTGGG | AAAATAAGGCTAGTCCGTTATCAAC | | | | | |
| | TACGA | TTGAAAAAGTGGCACCGAGTCGGT | | | | | |
| | | GC | | | | | |
| FANCF, nicking sgRNA | GGGGTCCC | GTTTTAGAGCTAGAAATAGCAAGTT | | | | | TTTTTT |
| | AGGTGCTG | AAAATAAGGCTAGTCCGTTATCAAC | | | | | |
| | ACGT | TTGAAAAAGTGGCACCGAGTCGGT | | | | | |
| | | GC | | | | | |
| PRNP, nicking sgRNA | GCATGTTTT | GTTTTAGAGCTAGAAATAGCAAGTT | | | | | TTTTTT |
| | CACGATAG | AAAATAAGGCTAGTCCGTTATCAAC | | | | | |
| | TAA | TTGAAAAAGTGGCACCGAGTCGGT | | | | | |
| | | GC | | | | | |
| RNF2, +5 G to T | GTCATCTTA | GTTTTAGAGCTAGAAATAGCAAGTT | AACGAACACATCAG | GTAATGAC | | | TTTTTT |
| | GTCATTAC | AAAATAAGGCTAGTCCGTTATCAAC | | TAAGATG | | | |
| | CTG | TTGAAAAAGTGGCACCGAGTCGGT | | | | | |
| | | GC | | | | | |
| Rosa26, attB insertion- | GCTACTGTT | GTTTTAGAGCTAGAAATAGCAAGTT | ATGATCCTGACGACG | TTAGAGTG | CAGTAG | CGCGGTTCTATCTA | TTTTTT |
| twinPE-pegRNA1 | CACTCTAA | AAAATAAGGCTAGTCCGTTATCAAC | GAGACCGCCGTCGTC | AA | | GTTACGCGTTAAA | |
| | CAG | TTGAAAAAGTGGCACCGAGTCGGT | GACAAGCC | | | CCAACTAGAA | |
| | | GC | | | | | |
| Rosa26, attB insertion- | GAATCTGC | GTTTTAGAGCTAGAAATAGCAAGTT | GGCTTGTCGACGACG | GATATACT | CAGATT | CGCGGTTCTATCTA | TTTTTT |
| twinPE-pegRNA2 | TAGTATAT | AAAATAAGGCTAGTCCGTTATCAAC | GCGGTCTCCGTCGTC | AG | | GTTACGCGTTAAA | |
| | CCGT | TTGAAAAAGTGGCACCGAGTCGGT | AGGATCAT | | | CCAACTAGAA | |
| | | GC | | | | | |
| CCR5 edit 1-twinPE- | GAAGTGTG | GTTTTAGAGCTAGAAATAGCAAGTT | ATGATCCTGACGACG | CCCAAGTG | | CGCGGTTCTATCTA | TTTTTT |
| pegRNA1 | ATCACTTG | AAAATAAGGCTAGTCCGTTATCAAC | GAGACCGCCGTCGTC | ATC | | GTTACGCGTTAAA | |
| | GGTGG | TTGAAAAAGTGGCACCGAGTCGGT | GACAAGCC | | | CCAACTAGAA | |
| | | GC | | | | | |
| CCR5 edit 1-twinPE- | GTATGGAA | GTTTTAGAGCTAGAAATAGCAAGTT | GGCTTGTCGACGACG | GCTCTCATT | | CGCGGTTCTATCTA | TTTTTT |
| pegRNA2 | AATGAGAG | AAAATAAGGCTAGTCCGTTATCAAC | GCGGTCTCCGTCGTC | TTC | | GTTACGCGTTAAA | |
| | CTGC | TTGAAAAAGTGGCACCGAGTCGGT | AGGATCAT | | | CCAACTAGAA | |
| | | GC | | | | | |
| CCR5 edit 2-twinPE- | GACCCCTC | GTTTTAGAGCTAGAAATAGCAAGTT | GGCTTGTCGACGACG | TGAAATAC | | | TTTTTT |
| pegRNA1 | AGTATTTC | AAAATAAGGCTAGTCCGTTATCAAC | GCGGTCTCCGTCGTC | TG | | | |
| | AGCT | TTGAAAAAGTGGCACCGAGTCGGT | AGGATCAT | | | | |
| | | GC | | | | | |
| CCR5 edit 2-twinPE- | GAAAAGAC | GTTTTAGAGCTAGAAATAGCAAGTT | ATGATCCTGACGACG | GTGCTTGA | | | TTTTTT |
| pegRNA2 | ATCAAGCA | AAAATAAGGCTAGTCCGTTATCAAC | GAGACCGCCGTCGTC | TGTC | | | |
| | CAGA | TTGAAAAAGTGGCACCGAGTCGGT | GACAAGCC | | | | |
| | | GC | | | | | |
| EMX1, +5 G to T (evopreQ1) | GAGTCCGA | GTTTTAGAGCTAGAAATAGCAAGTT | GTGATGGGAGCACTT | TTCTTCTGC | AACAATC | TTGACGCGGTTCT | TTTTTT |
| | GCAGAAGA | AAAATAAGGCTAGTCCGTTATCAAC | C | TCGGA | T | ATCTAGTTACGCG | |
| | AGAA | TTGAAAAAGTGGCACCGAGTCGGT | | | | TTAAACCAACTAG | |
| | | GC | | | | AAA | |
| VEGFA, +5 G to T | GATGTCTG | GTTTTAGAGCTAGAAATAGCAAGTT | AATGTGCCATCTGGA | TCTGGCCT | ACAATCT | TTGACGCGGTTCT | TTTTTT |
| (evopreQ1) | CAGGCCAG | AAAATAAGGCTAGTCCGTTATCAAC | GCACTCA | GCAGA | C | ATCTAGTTACGCG | |
| | ATGA | TTGAAAAAGTGGCACCGAGTCGGT | | | | TTAAACCAACTAG | |
| | | GC | | | | AAA | |
| RNF2, +5 G to T | GTCATCTTA | GTTTTAGAGCTAGAAATAGCAAGTT | AACGAACACATCAG | GTAATGAC | | | TTTTTT |
| | GTCATTAC | AAAATAAGGCTAGTCCGTTATCAAC | | TAAGATG | | | |
| | CTG | TTGAAAAAGTGGCACCGAGTCGGT | | | | | |
| | | GC | | | | | |
| FANCF, +5 G to T (mpknot) | GGAATCCC | GTTTTAGAGCTAGAAATAGCAAGTT | GGAAAAGCGATCAA | GCTGCAGA | CAATCAC | GGGTCAGGAGCCC | TTTTTT |
| | TTCTGCAG | AAAATAAGGCTAGTCCGTTATCAAC | GGT | AGGGA | T | CCCCCCTGAACCC | |
| | CACC | TTGAAAAAGTGGCACCGAGTCGGT | | | | AGGATAACCCTCA | |
| | | GC | | | | AAGTCGGGGGGCA | |
| | | | | | | ACCC | |
| PRNP, +6 G to T (evopreQ1) | GCAGTGGT | GTTTTAGAGCTAGAAATAGCAAGTT | ATGTAGACGCCA | AGGCCCCC | TAGACAC | CGCGGTTCTATCTA | TTTTTT |
| | GGGGGGCC | AAAATAAGGCTAGTCCGTTATCAAC | | CACC | A | GTTACGCGTTAAA | |
| | TTGG | TTGAAAAAGTGGCACCGAGTCGGT | | | | CCAACTAGAA | |
| | | GC | | | | | |
| RNF2, nicking sgRNA | GTCAACCA | GTTTTAGAGCTAGAAATAGCAAGTT | | | | | TTTTTT |
| | TTAAGCAA | AAAATAAGGCTAGTCCGTTATCAAC | | | | | |
| | AACAT | TTGAAAAAGTGGCACCGAGTCGGT | | | | | |
| | | GC | | | | | |
| EMX1, nicking sgRNA | GCCGTTTGT | GTTTTAGAGCTAGAAATAGCAAGTT | | | | | TTTTTT |
| | ACTTTGTCC | AAAATAAGGCTAGTCCGTTATCAAC | | | | | |
| | TC | TTGAAAAAGTGGCACCGAGTCGGT | | | | | |
| | | GC | | | | | |
| VEGFA, nicking sgRNA | GAGCCCAG | GTTTTAGAGCTAGAAATAGCAAGTT | | | | | TTTTTT |
| | GGCTGGGC | AAAATAAGGCTAGTCCGTTATCAAC | | | | | |
| | ACAG | TTGAAAAAGTGGCACCGAGTCGGT | | | | | |
| | | GC | | | | | |
| FANCF, nicking sgRNA | GGGGTCCC | GTTTTAGAGCTAGAAATAGCAAGTT | | | | | TTTTTT |
| | AGGTGCTG | AAAATAAGGCTAGTCCGTTATCAAC | | | | | |
| | ACGT | TTGAAAAAGTGGCACCGAGTCGGT | | | | | |
| | | GC | | | | | |
| PRNP, nicking sgRNA | GCATGTTTT | GTTTTAGAGCTAGAAATAGCAAGTT | | | | | TTTTTT |
| | CACGATAG | AAAATAAGGCTAGTCCGTTATCAAC | | | | | |
| | TAA | TTGAAAAAGTGGCACCGAGTCGGT | | | | | |
| | | GC | | | | | |
| All pegRNA, epegRNAs, and | | | | | | | |
| nicking sgRNAs used are | | | | | | | |
| noted in Table S14 | | | | | | | |
| Correction of 1-bp deletion in | GCTCGCGA | GTTTTAGAGCTAGAAATAGCAAGTT | AGACTCATGCTCAAG | GCCAACTG | | | TTTTTT |
| T7 RNAP, (+6 ins A, v1 | ACAGTTGG | AAAATAAGGCTAGTCCGTTATCAAC | G | T | | | |
| circuit) | CCCT | TTGAAAAAGTGGCACCGAGTCGGT | | | | | |
| | | GC | | | | | |
| All pegRNA, epegRNAs, and | | | | | | | |
| nicking sgRNAs as in FIG. | | | | | | | |
| 6B | | | | | | | |
| All pegRNA, epegRNAs, and | | | | | | | |
| nicking sgRNAs as in FIG. | | | | | | | |
| 6D | | | | | | | |
| Rosa26, attB insertion- | GTCTACTGT | GTTTTAGAGCTAGAAATAGCAAGTT | ATGATCCTGACGACG | TTAGAGTG | CAGTAG | CGCGGTTCTATCTA | TTTTTT |
| twinPE-pegRNA1 tevoPreQ1 | TCACTCTA | AAAATAAGGCTAGTCCGTTATCAAC | GAGACCGCCGTCGTC | AA | | GTTACGCGTTAAA | |
| | ACAG | TTGAAAAAGTGGCACCGAGTCGGT | GACAAGCC | | | CCAACTAGAA | |
| | | GC | | | | | |
| Rosa26, attB insertion- | GTAATCTG | GTTTTAGAGCTAGAAATAGCAAGTT | GGCTTGTCGACGACG | GATATACT | CAGATT | CGCGGTTCTATCTA | TTTTTT |
| twinPE-pegRNA2 tevoPreQ1 | CTAGTATA | AAAATAAGGCTAGTCCGTTATCAAC | GCGGTCTCCGTCGTC | AG | | GTTACGCGTTAAA | |
| | TCCGT | TTGAAAAAGTGGCACCGAGTCGGT | AGGATCAT | | | CCAACTAGAA | |
| | | GC | | | | | |
| mDnmt1, loxP insertion | GCGGGCTG | GTTTTAGAGCTAGAAATAGCAAGTT | GAGCTGTTCTGTCGT | CGAACAGC | | CGCGGTTCTATCTA | TTTTTT |
| pegRNA, tevoPreQ1 | GAGCTGTT | AAAATAAGGCTAGTCCGTTATCAAC | CTGCAACCTGCAAGA | T | | GTTACGCGTTAAA | |
| | CGCGC | TTGAAAAAGTGGCACCGAGTCGGT | TGCCAGCGATTGTTA | | | CCAACTAGAA | |
| | | GC | TAACTTCGTATAGCA | | | | |
| | | | TACATTATACGAAGT | | | | |
| | | | TATCG | | | | |
| mDnmt1, nicking sgRNA | GCCGCGCG | GTTTTAGAGCTAGAAATAGCAAGTT | | | | | TTTTTT |
| | CGCGAAAA | AAAATAAGGCTAGTCCGTTATCAAC | | | | | |
| | AGCCG | TTGAAAAAGTGGCACCGAGTCGGT | | | | | |
| | | GC | | | | | |

### EMBODIMENTS

The following embodiments are within the scope of the present disclosure. Furthermore, the disclosure encompasses all variations, combinations, and permutations of these embodiments in which one or more limitations, elements, clauses, and descriptive terms from one or more of the listed embodiments is introduced into another listed embodiment in this section. For example, any listed embodiment that is dependent on another embodiment can be modified to include one or more limitations found in any other listed embodiment in this section that is dependent on the same base embodiment. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the invention or aspects of the invention consist, or consist essentially of, such elements and/or features. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Embodiment 1. A reverse transcriptase variant having at least 80%, at least 85%, at least 90%, at least 95%, at least 96%, at least 97%, at least 98%, or at least 99% sequence identity with SEQ ID NO: 1, wherein the reverse transcriptase variant comprises amino acid substitutions at positions 70, 72, 87, 102, 106, 118, 128, 158, 269, 363, 413, and 492 relative to SEQ ID NO: 1, or corresponding substitutions in a homologous sequence.

Embodiment 2. The reverse transcriptase variant of embodiment 1, wherein the amino acid substitutions comprise P70T, G72V, S87G, M102I, K106R, K118R, I128V, L158Q, F269L, A363V, K413E, and S492N relative to SEQ ID NO: 1.

Embodiment 3. The reverse transcriptase variant of embodiment 1 or 2 further comprising amino acid substitutions at positions 188, 260, 297, and 288 relative to SEQ ID NO: 1.

Embodiment 4. The reverse transcriptase variant of embodiment 3, wherein the amino acid substitutions comprise S188K, I260L, S297Q, and R288Q relative to SEQ ID NO: 1.

Embodiment 5. The reverse transcriptase variant of embodiment 1 or 2, wherein the reverse transcriptase variant comprises SEQ ID NO: 25.

Embodiment 6. The reverse transcriptase variant of embodiment 1 or 2, wherein the reverse transcriptase variant comprises SEQ ID NO: 26.

Embodiment 7. A reverse transcriptase variant having at least 80%, at least 85%, at least 90%, at least 95%, at least 96%, at least 97%, at least 98%, or at least 99% sequence identity with SEQ ID NO: 30, wherein the reverse transcriptase variant comprises amino acid substitutions at positions 128 and 200 relative to SEQ ID NO: 30, or corresponding substitutions in a homologous sequence.

Embodiment 8. The reverse transcriptase variant of embodiment 7, wherein the amino acid substitutions comprise T128N and D200C relative to SEQ ID NO: 30.

Embodiment 9. The reverse transcriptase variant of embodiment 7 or 8 further comprising amino acid substitutions at positions 223, 306, 313, and 330 relative to SEQ ID NO: 30.

Embodiment 10. The reverse transcriptase variant of embodiment 9, wherein the amino acid substitutions comprise V223Y, T306K, W313F, and T330P relative to SEQ ID NO: 30.

Embodiment 11. A reverse transcriptase variant having at least 80%, at least 85%, at least 90%, at least 95%, at least 96%, at least 97%, at least 98%, or at least 99% sequence identity with SEQ ID NO: 30, wherein the reverse transcriptase variant comprises the amino acid substitutions T128N and V223M; T128N and V223Y; T128F and V223M; or D200C and V223M relative to SEQ ID NO: 30, or corresponding substitutions in a homologous sequence, optionally wherein the reverse transcriptase variant further comprises one or more of the amino acid substitutions D200N, T306K, W313F, T330P, and L603W relative to SEQ ID NO: 30.

Embodiment 12. A reverse transcriptase variant having at least 80%, at least 85%, at least 90%, at least 95%, at least 96%, at least 97%, at least 98%, or at least 99% sequence identity with SEQ ID NO: 30, wherein the reverse transcriptase variant comprises amino acid substitutions at positions 128, 129, 196, 200, and 223 relative to SEQ ID NO: 30, or corresponding substitutions in a homologous sequence.

Embodiment 13. The reverse transcriptase variant of embodiment 12, wherein the amino acid substitutions comprise (i) T128N; (ii) V129A or V129G; (iii) P196S, P196T, or P196F; (iv) D200S or D200Y; and (v) V223A, V223M, V223L, or V223E relative to SEQ ID NO: 30, optionally wherein the reverse transcriptase variant further comprises one or more of the amino acid substitutions D200N, T306K, W313F, T330P, and L603W relative to SEQ ID NO: 30.

Embodiment 14. The reverse transcriptase variant of any one of embodiments 7-13, wherein the reverse transcriptase variant comprises a C-terminal truncation of part or all of the RNaseH domain of SEQ ID NO: 30.

Embodiment 15. The reverse transcriptase variant of embodiment 14, wherein the C-terminal truncation is between amino acid positions D497 and I498 of SEQ ID NO: 30.

Embodiment 16. The reverse transcriptase variant of embodiment 14, wherein the C-terminal truncation is between amino acid positions Q492 and H493 or between amino acid positions L491 and Q492.

Embodiment 17. The reverse transcriptase variant of embodiment 14, wherein the C-terminal truncation is between amino acid positions A502 and H503 or between amino acid positions H503 and G504.

Embodiment 18. The reverse transcriptase variant of any one of embodiments 7-15, wherein the reverse transcriptase comprises an amino acid having at least 80%, at least 85%, at least 90%, at least 95%, at least 96%, at least 97%, at least 98%, or at least 99% sequence identity with SEQ ID NO: 27.

Embodiment 19. The reverse transcriptase variant of embodiment 18 comprising the amino acid sequence as set forth in SEQ ID NO: 27.

Embodiment 20. The reverse transcriptase variant of embodiment 18, wherein the reverse transcriptase variant consists of the amino acid sequence as set forth in SEQ ID NO: 27.

Embodiment 21. A reverse transcriptase variant having at least 80%, at least 85%, at least 90%, at least 95%, at least 96%, at least 97%, at least 98%, or at least 99% sequence identity with SEQ ID NO: 30, wherein the reverse transcriptase variant comprises all amino acid residues indicated in a single row of a table in any one of FIGS. 26, 27, 28A, 28B, and 28C as corresponding positions indicated in the same table relative to SEQ ID NO: 29, or corresponding substitutions in a homologous sequence.

Embodiment 22. A reverse transcriptase variant having at least 80%, at least 85%, at least 90%, at least 95%, at least 96%, at least 97%, at least 98%, or at least 99% sequence identity with SEQ ID NO: 30, wherein the reverse transcriptase variant comprises one or more amino acid substitutions selected from the group consisting of N200A, N200C, N200E, N200H, N200I, N200L, N200M, N200Q, N200R, N200T, N200V, N200W, N200S, N200Y, P196F, P196S, P196T, T128F, T128N, V129A, V129G, V223A, V223C, V223E, V223F, V223G, V223H, V223I, V223K, V223M, V223P, V223Q, V223R, V223S, V223T, V223W, and V223Y relative to SEQ ID NO: 30, or corresponding substitutions in a homologous sequence.

Embodiment 23. A Cas9 variant having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 775 and 918 relative to SEQ ID NO: 2, or corresponding substitutions in a homologous sequence.

Embodiment 24. The Cas9 variant of embodiment 23, wherein the amino acid substitutions comprise K775R and K918A relative to SEQ ID NO: 2.

Embodiment 25. The Cas9 variant of embodiment 24, wherein the Cas9 variant comprises an amino acid having at least 80%, at least 85%, at least 90%, at least 95%, at least 96%, at least 97%, at least 98%, or at least 99% sequence identity with SEQ ID NO: 28.

Embodiment 26. The Cas9 variant of embodiment 25 comprising the amino acid sequence as set forth in SEQ ID NO: 28.

Embodiment 27. A Cas9 variant having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 99, 471, 632, 645, and 721 relative to SEQ ID NO: 2, or corresponding substitutions in a homologous sequence.

Embodiment 28. The Cas9 variant of embodiment 27, wherein the amino acid substitutions comprise H99R, E471K, I632V, D645N, and H721Y relative to SEQ ID NO: 2.

Embodiment 29. The Cas9 variant of embodiment 27 or 28 further comprising an amino acid substitution at position 654 relative to SEQ ID NO: 2.

Embodiment 30. The Cas9 variant of embodiment 29, wherein the amino acid substitution comprises R654C relative to SEQ ID NO: 2.

Embodiment 31. The Cas9 variant of any one of embodiments 27-30 further comprising an amino acid substitution at position 918 relative to SEQ ID NO: 2.

Embodiment 32. The Cas9 variant of embodiment 31, wherein the amino acid substitution comprises K918A relative to SEQ ID NO: 2.

Embodiment 33. The Cas9 variant of embodiment 31 comprising an amino acid having at least 80%, at least 85%, at least 90%, at least 95%, at least 96%, at least 97%, at least 98%, or at least 99% sequence identity with SEQ ID NO: 48.

Embodiment 34. The Cas9 variant of embodiment 33 comprising the amino acid sequence as set forth in SEQ ID SEQ ID NO: 48.

Embodiment 35. The Cas9 variant of embodiment 30 comprising an amino acid having at least 80%, at least 85%, at least 90%, at least 95%, at least 96%, at least 97%, at least 98%, or at least 99% sequence identity with SEQ ID NO: 49.

Embodiment 36. The Cas9 variant of embodiment 35 comprising the amino acid sequence as set forth in SEQ ID NO: 49.

Embodiment 37. A Cas9 variant having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 99, 471, and 632 relative to SEQ ID NO: 2, or corresponding substitutions in a homologous sequence.

Embodiment 38. The Cas9 variant of embodiment 37, wherein the amino acid substitutions comprise H99R, E471K, and I632V relative to SEQ ID NO: 2.

Embodiment 39. The Cas9 variant of embodiment 37 or 38 further comprising an amino acid substitution at position 721 relative to SEQ ID NO: 2.

Embodiment 40. The Cas9 variant of embodiment 39, wherein the amino acid substitution is H721Y relative to SEQ ID NO: 2.

Embodiment 41. A Cas9 variant having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 471 and 918 relative to SEQ ID NO: 2, or corresponding substitutions in a homologous sequence.

Embodiment 42. The Cas9 variant of embodiment 41, wherein the amino acid substitutions comprise E471K and K918A relative to SEQ ID NO: 2.

Embodiment 43. A Cas9 variant having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 753 and 1151 relative to SEQ ID NO: 2, or corresponding substitutions in a homologous sequence.

Embodiment 44. The Cas9 variant of embodiment 43, wherein the amino acid substitutions comprise R753G and K1151E relative to SEQ ID NO: 2.

Embodiment 45. A Cas9 variant having at least 80%, at least 85%, at least 90%, at least 95%, at least 96%, at least 97%, at least 98%, or at least 99% sequence identity with SEQ ID NO: 2, wherein the Cas9 variant comprises one or more amino acid substitutions at positions selected from the group consisting of 260, 298, 395, 769, 778, 1014, 1034, 1100, 1106, 1138, 1152, and 1320 relative to SEQ ID NO: 2, or corresponding substitutions in a homologous sequence.

Embodiment 46. The Cas9 variant of embodiment 45, wherein the one or more amino acid substitutions are selected from the group consisting of E260K, D298N, R395C, T769P, R778Q, K1014E, A1034E, V1100I, S1106F, T1138A, G1152E, and A1320T.

Embodiment 47. The Cas9 variant of embodiment 44 or 45 further comprising one or more additional amino acid substitutions at positions selected from the group consisting of 102, 753, 804, and 1003 relative to SEQ ID NO: 2.

Embodiment 48. The Cas9 variant of embodiment 47, wherein the one or more additional amino acid substitutions are selected from the group consisting of E102K, R753G, T804A, and K1003R.

Embodiment 49. The Cas9 variant of any one of embodiments 44-48 comprising amino acid substitutions at any one of the groups of positions:
102, 395, 753, 778, and 1100;
753, 769, 1034, and 1320;
298, 753, 1034, and 1138;
102, 260, 395, 753, 778, 804, 1003, 1100, 1106, and 1152; or
102, 260, 395, 753, 778, 804, 1003, 1014, 1100, 1106, and 1152;
relative to SEQ ID NO: 2.

Embodiment 50. The Cas9 variant of any one of embodiments 44-49 comprising amino acid substitutions at any one of the groups of positions:
E102K, R395C, R753G, R778Q, and V1100I;
R753G, T769P, A1034E, and A1320T;
D298N, R753G, A1034E, and T1138A;
E102K, E260K, R395C, R753C, R778Q, T804A, K1003R, V1100I, S1106F, and G1152E; or
E102K, E260K, R395C, R753G, R778Q, T804A, K1003R, K1014E, V1100I, S1106F, and G1152E;
relative to SEQ ID NO: 2.

Embodiment 51. A Cas9 variant having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 23 and 754 relative to SEQ ID NO: 2, or corresponding substitutions in a homologous sequence.

Embodiment 52. The Cas9 variant of embodiment 51, wherein the amino acid substitutions are D23G and H754R.

Embodiment 53. A prime editor comprising a reverse transcriptase variant of any one of embodiments 1-22 and a nucleic acid-programmable DNA-binding protein (napDNAbp).

Embodiment 54. The prime editor of embodiment 53, wherein the napDNAbp comprises a Cas9 protein.

Embodiment 55. The prime editor of embodiment 54, wherein the Cas9 protein is a Cas9 nickase, optionally wherein the Cas9 nickase comprises a mutation in a HNH domain that inactivates nuclease activity of the HNH domain.

Embodiment 56. The prime editor of any one of embodiments 53-55, wherein the napDNAbp comprises a Cas9 variant of any one of embodiments 23-52.

Embodiment 57. The prime editor of any one of embodiments 53-56, wherein the napDNAbp comprises a Cas9 protein of any one of SEQ ID NOs: 10, 2, 6, 8, 9, 12-24, or 133, or an amino acid sequence at least 80%, at least 85%, at least 90%, at least 95%, at least 96%, at least 97%, at least 98%, or at least 99% identical to any one of SEQ ID NOs: 10, 2, 6, 8, 9, 12-24, or 133.

Embodiment 58. The prime editor of any one of embodiments 53-57, wherein the napDNAbp comprises SEQ ID NO: 10.

Embodiment 59. The prime editor of any one of embodiments 53-57, wherein the napDNAbp comprises SEQ ID NO: 11.

Embodiment 60. The prime editor of any one of embodiments 53-57, wherein the napDNAbp comprises a Cas9 protein of SEQ ID NO: 133, or an amino acid sequence at least 80%, at least 85%, at least 90%, at least 95%, at least 96%, at least 97%, at least 98%, or at least 99% identical to SEQ ID NO: 133.

Embodiment 61. A prime editor comprising a Cas9 variant of any one of embodiments 23-52 and a polymerase.

Embodiment 62. The prime editor of embodiment 61, wherein the polymerase is a reverse transcriptase.

Embodiment 63. The prime editor of embodiment 62, wherein the reverse transcriptase is a reverse transcriptase variant of any one of embodiments 1-13.

Embodiment 64. The prime editor of embodiment 62, wherein the reverse transcriptase comprises a sequence having at least 80%, at least 85%, at least 90%, at least 95%, at least 96%, at least 97%, at least 98%, or at least 99% sequence identity with SEQ ID NO: 7, wherein the reverse transcriptase comprises amino acid substitutions at positions 60, 87, 165, 243, 267, 279, 318, and 343 relative to SEQ ID NO: 7, or corresponding positions in a homologous sequence.

Embodiment 65. The prime editor of embodiment 64, wherein the amino acid substitutions comprise E60K, K87E, E165D, D243N, R267I, E279K, K318E, and K343N relative to SEQ ID NO: 7.

Embodiment 66. The prime editor of any one of embodiments 53-65, wherein the napDNAbp and the reverse transcriptase are provided in trans or are not fused to one another.

Embodiment 67. The prime editor of any one of embodiments 53-65, wherein the prime editor comprises a fusion protein comprising the napDNAbp and the reverse transcriptase covalently connected to one another.

Embodiment 68. The prime editor of embodiment 67, wherein the napDNAbp and the reverse transcriptase are fused via a linker.

Embodiment 69. The prime editor of embodiment 68, wherein the linker comprises any one of SEQ ID NOs: 80-93.

Embodiment 70. The prime editor of any one of embodiments 53-69 further comprising a nuclear localization sequence (NLS).

Embodiment 71. A prime editor comprising a Cas9 protein and a reverse transcriptase variant having at least 80%, at least 85%, at least 90%, at least 95%, at least 96%, at least 97%, at least 98%, or at least 99% sequence identity with SEQ ID NO: 1, wherein the reverse transcriptase variant comprises the amino acid substitutions P70T, G72V, S87G, M102I, K106R, K118R, I128V, L158Q, F269L, A363V, K413E, and S492N relative to SEQ ID NO: 1.

Embodiment 72. A prime editor comprising a Cas9 protein and a reverse transcriptase variant having at least 80%, at least 85%, at least 90%, at least 95%, at least 96%, at least 97%, at least 98%, or at least 99% sequence identity with SEQ ID NO: 1, wherein the reverse transcriptase variant comprises the amino acid substitutions P70T, G72V, S87G, M102I, K106R, K118R, I128V, L158Q, S188K, I260L, F269L, R288Q, S297Q, A363V, K413E, and S492N relative to SEQ ID NO: 1.

Embodiment 73. A prime editor comprising a Cas9 protein and a reverse transcriptase variant having at least 80%, at least 85%, at least 90%, at least 95%, at least 96%, at least 97%, at least 98%, or at least 99% sequence identity with SEQ ID NO: 30, wherein the reverse transcriptase variant comprises the amino acid substitutions T128N, D200C, and V223Y relative to SEQ ID NO: 30.

Embodiment 74. The prime editor of any one of embodiments 71-73, wherein the Cas9 protein comprises a Cas9 variant having at least 80%, at least 85%, at least 90%, at least 95%, at least 96%, at least 97%, at least 98%, or at least 99% sequence identity with SEQ ID NO: 2, wherein the Cas9 variant comprises the amino acid substitutions K775R and K918A; H99R, E471K, I632V, D645N, H721Y, and K918A; or H99R, E471K, I632V, D645N, R654C, and H721Y relative to SEQ ID NO: 2.

Embodiment 75. A prime editor comprising a reverse transcriptase and a Cas9 variant having at least 80%, at least 85%, at least 90%, at least 95%, at least 96%, at least 97%, at least 98%, or at least 99% sequence identity with SEQ ID NO: 2, wherein the Cas9 variant comprises the amino acid substitutions K775R and K918A relative to SEQ ID NO: 2.

Embodiment 76. A prime editor comprising a reverse transcriptase and a Cas9 variant having at least 80%, at least 85%, at least 90%, at least 95%, at least 96%, at least 97%, at least 98%, or at least 99% sequence identity with SEQ ID NO: 2, wherein the Cas9 variant comprises the amino acid substitutions H99R, E471K, I632V, D645N, H721Y, and K918A relative to SEQ ID NO: 2.

Embodiment 77. A prime editor comprising a reverse transcriptase and a Cas9 variant having at least 80%, at least 85%, at least 90%, at least 95%, at least 96%, at least 97%, at least 98%, or at least 99% sequence identity with SEQ ID NO: 2, wherein the Cas9 variant comprises the amino acid substitutions H99R, E471K, I632V, D645N, R654C, and H721Y relative to SEQ ID NO: 2.

Embodiment 78. The prime editor of any one of embodiments 75-77, wherein the reverse transcriptase comprises a reverse transcriptase variant having at least 80%, at least 85%, at least 90%, at least 95%, at least 96%, at least 97%, at least 98%, or at least 99% sequence identity with SEQ ID NO: 1, wherein the reverse transcriptase variant comprises the amino acid substitutions P70T, G72V, S87G, M102I, K106R, K118R, I128V, L158Q, F269L, A363V, K413E, and S492N; or P70T, G72V, S87G, M102I, K106R, K118R, I128V, L158Q, S188K, I260L, F269L, R288Q, S297Q, A363V, K413E, and S492N relative to SEQ ID NO: 1.

Embodiment 79. The prime editor of any one of embodiments 75-77, wherein the reverse transcriptase comprises a reverse transcriptase variant having at least 80%, at least 85%, at least 90%, at least 95%, at least 96%, at least 97%, at least 98%, or at least 99% sequence identity with SEQ ID NO: 30, wherein the reverse transcriptase variant comprises the amino acid substitutions T128N, D200C, and V223Y relative to SEQ ID NO: 30.

Embodiment 80. The prime editor of any one of embodiments 75-77, wherein the reverse transcriptase comprises a reverse transcriptase variant having at least 80%, at least 85%, at least 90%, at least 95%, at least 96%, at least 97%, at least 98%, or at least 99% sequence identity with SEQ ID NO: 7, wherein the reverse transcriptase comprises the amino acid substitutions E60K, K87E, E165D, D243N, R267I, E279K, K318E, and K343N relative to SEQ ID NO: 7.

Embodiment 81. A prime editor comprising a Cas9 variant of any one of SEQ ID NOs: 28, 48, or 49 and a reverse transcriptase variant of any one of SEQ ID NOs: 25-27 or 50, or a Cas9 variant at least 80%, at least 85%, at least 90%, at least 95%, at least 96%, at least 97%, at least 98%, or at least 99% to any one of SEQ ID NOs: 28, 48, or 49 and a reverse transcriptase variant at least 80%, at least 85%, at least 90%, at least 95%, at least 96%, at least 97%, at least 98%, or at least 99% to any one of SEQ ID NOs: 25-27 or 50.

Embodiment 82. The prime editor of any one of embodiments 67-81, wherein the prime editor comprises the fusion protein comprising the structure NH2-[bipartite NLS]-[Cas9]-[linker]-[reverse transcriptase]-[bipartite NLS]-[NLS].

Embodiment 83. The prime editor of any one of embodiments 67-82, wherein the prime editor comprises the fusion protein architecture of PEmax.

Embodiment 84. The prime editor of any one of embodiments 67-82, wherein the prime editor is smaller in size than PE2, and wherein the prime editor has an editing efficiency comparable to that of PE2.

Embodiment 85. The prime editor of any one of embodiments 67-82, wherein the fusion protein comprises an amino acid sequence having at least 80%, at least 85%, at least 90%, at least 95%, at least 96%, at least 97%, at least 98%, or at least 99% sequence identity to SEQ ID NO: 135, or wherein the prime editor comprises the amino acid sequence of SEQ ID NO: 135.

Embodiment 86. The prime editor of any one of embodiments 67-82 wherein the fusion protein comprises an amino acid sequence having at least 80%, at least 85%, at least 90%, at least 95%, at least 96%, at least 97%, at least 98%, or at least 99% sequence identity to SEQ ID NO: 136, or wherein the prime editor comprises the amino acid sequence of SEQ ID NO: 136.

Embodiment 87. The prime editor of any one of embodiments 67-82 wherein the fusion protein comprises an amino acid sequence having at least 80%, at least 85%, at least 90%, at least 95%, at least 96%, at least 97%, at least 98%, or at least 99% sequence identity to SEQ ID NO: 999, or wherein the prime editor comprises the amino acid sequence of SEQ ID NO: 999.

Embodiment 88. The prime editor of any one of embodiments 67-82 wherein the fusion protein comprises an amino acid sequence having at least 80%, at least 85%, at least 90%, at least 95%, at least 96%, at least 97%, at least 98%, or at least 99% sequence identity to SEQ ID NO: 1000, or wherein the prime editor comprises the amino acid sequence of SEQ ID NO: 1000.

Embodiment 89. The prime editor of any one of embodiments 53-88, wherein the prime editor has an increased editing efficiency compared to PEmax for edits that require structured pegRNA reverse transcriptase templates (RTTs).

Embodiment 90. A fusion protein comprising a Cas9 variant of any one of embodiments 23-52 and an effector domain.

Embodiment 91. The fusion protein of embodiment 90, wherein the effector domain comprises nuclease activity, nickase activity, recombinase activity, deaminase activity, methyltransferase activity, methylase activity, acetylase activity, acetyltransferase activity, transcriptional activation activity, transcriptional repression activity, or polymerase activity.

Embodiment 92. A reverse transcriptase variant comprising the sequence of any one of SEQ ID NOs: 25-27 or 50, or a sequence at least 80%, at least 85%, at least 90%, at least 95%, at least 96%, at least 97%, at least 98%, or at least 99% to any one of SEQ ID NOs: 25-27 or 50.

Embodiment 93. A Cas9 variant comprising the sequence of any one of SEQ ID NOs: 28, 48, 49, or X, or a sequence at least 80%, at least 85%, at least 90%, at least 95%, at least 96%, at least 97%, at least 98%, or at least 99% to any one of SEQ ID NOs: 28, 48, 49, or X.

Embodiment 94. A prime editor comprising the sequence of any one of SEQ ID NOs: 134-137, 999, and 1000, or a sequence at least 80%, at least 85%, at least 90%, at least 95%, at least 96%, at least 97%, at least 98%, or at least 99% to any one of SEQ ID NOs: 134-137, 999, and 1000.

Embodiment 95. A prime editing system comprising the prime editor of any one of embodiments 53-89 or 94.

Embodiment 96. A prime editing system comprising:
(a) an N-terminal extein comprising an N-terminal fragment of a fusion protein of any one of embodiments 67-88 and an N-intein;
(b) a C-terminal extein comprising a C-terminal fragment of the fusion protein in (a) a C-intein;
wherein the N-intein and the C-intein of the N-terminal and C-terminal exteins are capable of self-excision to join the N-terminal fragment and the C-terminal fragment to form the fusion protein.

Embodiment 97. The prime editing system of embodiment 96, wherein the N-terminal fragment comprises amino acids 1-1024 of the Cas9 and wherein the C-terminal fragment comprises amino acids 1025-1368 of the Cas9, optionally wherein the C-terminal fragment comprises amino acids Cysteine-Phenylalanine-Asparagine at positions 1025-1027 of the Cas9, wherein positioning of the Cas9 is relative to SEQ ID NO: 2.

Embodiment 98. The prime editing system of embodiment 96, wherein the N-terminal fragment comprises amino acids 1-844 of the Cas9 and wherein the C-terminal fragment comprises amino acids 845-1368 of the Cas9, optionally wherein the C-terminal fragment comprises amino acids Cysteine-Phenylalanine-Asparagine at positions 845-847 of the Cas9, wherein positioning of the Cas9 is relative to SEQ ID NO: 2.

Embodiment 99. A complex comprising the (a) prime editor of any one of embodiments 53-89 or 94, or the fusion protein of embodiment 90 or 91 and (b) a pegRNA, optionally wherein the pegRNA is an epegRNA.

Embodiment 100. The prime editing system of any one of embodiments 95-98, wherein the prime editing system further comprises a prime editing guide RNA (PEgRNA), wherein the PEgRNA comprising a spacer, a gRNA core, and an extension arm comprising a DNA synthesis template and a primer binding site, optionally wherein the prime editing system further comprises a nicking guide RNA (ngRNA) comprising a ngRNA spacer and a ngRNA core.

Embodiment 101. The prime editing system of embodiment 100, wherein the DNA synthesis template is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length.

Embodiment 102. The prime editing system of embodiment 100, wherein the DNA synthesis template is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length.

Embodiment 103. The prime editing system of any one of embodiments 100-102, wherein the DNA synthesis template is at least 40 nucleotides in length.

Embodiment 104. The prime editing system of any one of embodiments 100-102, wherein the DNA synthesis template is at least 30 nucleotides in length, optionally wherein the DNA synthesis template is at least 31 nucleotides in length.

Embodiment 105. The prime editing system of any one of embodiments 100-102, wherein the DNA synthesis template is at least 35, 40, 42, 43, 44, 58, 69, 71, or 74 nucleotides in length, optionally wherein the DNA synthesis template is 35, 40, 42, 43, 44, 58, 69, 71 or 74 nucleotides in length.

Embodiment 106. The prime editing system of any one of embodiments 100-102, wherein the DNA synthesis template is at least 40, 42, 58, or 74 nucleotides in length, optionally wherein the DNA synthesis template is 40, 42, 58, or 74 nucleotides in length.

Embodiment 107. The prime editing system of any one of embodiments 100-106, wherein the free energy of folding of the extension arm is more than −23 kcal/mol as calculated by NUPACK free energy prediction.

Embodiment 108. The prime editing system of any one of embodiments 95-98, further comprising:
(a) a first prime editing guide RNA (first pegRNA) that comprises:
(i) a first spacer sequence that is complementary to a first binding site on a first strand of a double-stranded DNA sequence upstream of a target site relative to a second strand of the double stranded DNA sequence that is complementary to the first strand,
(ii) a first gRNA core that is capable of complexing with the prime editor, and
(iii) a first DNA synthesis template that encodes a first single-stranded DNA sequence,
and
(b) a second prime editing guide RNA (second pegRNA) that comprises
(i) a second spacer sequence that is complementary to a second binding site on the second strand of the double-stranded DNA sequence downstream of the target site relative to the second strand;
(ii) a second gRNA core that is capable of complexing with the prime editor, and
(iii) a second DNA synthesis template that encodes a second single-stranded DNA sequence;
wherein the first single-stranded DNA sequence and the second single-stranded DNA sequence are reverse complements over a region of complementarity of each single-stranded DNA sequence,
wherein the first single-stranded DNA sequence comprises a first edit compared to the second strand of the target site, and
wherein the second single-stranded DNA sequence comprises a second edit compared to the first strand of the target site.
Embodiment 109. The prime editing system of embodiment 108, wherein the region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides in length, optionally wherein the region of complementarity is 25, 45, 32, or 37 nucleotides in length.

Embodiment 110. The prime editing system of embodiment 109, wherein the first edit and the second edit together comprise an insertion, optionally wherein the insertion is at least 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, or 108 nucleotides in length, optionally wherein the insertion is 38, 43, 50, or 108 nt in length.

Embodiment 111. The prime editing system of embodiment 108, wherein the first DNA synthesis template is at least 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 65, 66, 67, 68, 69, 70, or 71 nucleotides in in length, optionally wherein the first DNA synthesis template is 34, 38, 43, 44, 69, or 71 nucleotides in length.

Embodiment 112. The prime editing system of embodiment 108, wherein the second DNA synthesis template is at least 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 65, 65, 66, 67, 68, 69, 70, or 71 nucleotides in in length, optionally wherein the second DNA synthesis template is 34, 38, 43, 44, 69, or 71 nucleotides in length.

Embodiment 113. The prime editing system of embodiment 110, wherein the insertion comprises a recombinase recognition site (RRS).

Embodiment 114. The prime editing system of embodiment 113, wherein the first DNA synthesis template encodes the RRS.

Embodiment 115. The prime editing system of embodiment 113, wherein the second DNA synthesis template encodes the RRS.

Embodiment 116. The prime editing system of any one of embodiments 113-115, wherein the recombinase recognition sequence is an attB sequence recognized by a Bxb1 recombinase.

Embodiment 117. The prime editing system of any one of embodiments 113-115, wherein the recombinase recognition sequence is an attP sequence recognized by a Bxb1 recombinase.

Embodiment 118. The prime editing system of any one of embodiments 113-115, wherein the recombinase recognition sequence is a loxP sequence recognized by a Cre recombinase.

Embodiment 119. The prime editing system of any one of embodiments 113-118, further comprising a recombinase.

Embodiment 120. The prime editing system of embodiment 119, wherein the recombinase is a bxb1 recombinase.

Embodiment 121. The prime editing system of embodiment 119, wherein the recombinase is a Cre recombinase.

Embodiment 122. The prime editing system of any one of embodiments 113-121, further comprising a DNA donor comprising a second RRS.

Embodiment 123. A polynucleotide encoding the reverse transcriptase variant of any one of embodiments 1-22 or 92.

Embodiment 124. A polynucleotide encoding the Cas9 variant of any one of embodiments 14-37 or 72.

Embodiment 125. One or more polynucleotides encoding the prime editor of any one of embodiments 53-89 or the fusion protein of embodiment 90 or 91.

Embodiment 126. One or more polynucleotides encoding the prime editor of any one of embodiments 53-89 or 94, the fusion protein of embodiment 90 or 91, or the prime editing system of any one of embodiments 95-98.

Embodiment 127. A vector comprising the polynucleotide of embodiment 123 and/or the polynucleotide of embodiment 124.

Embodiment 128. The vector of embodiment 127 further comprising a polynucleotide encoding a pegRNA.

Embodiment 129. One or more vectors comprising the one or more polynucleotides of embodiment 125 or 126.

Embodiment 130. The one or more vectors of embodiment 129, further comprising a polynucleotide encoding a pegRNA.

Embodiment 131. The one or more vectors of embodiment 129 or 130 comprising one or more polynucleotides encoding the N-terminal extein and the C-terminal extein of any one of embodiments 96-98, wherein the one or more vectors comprise a first vector and a second vector, wherein the first vector comprises a polynucleotide encoding the N-extein, wherein the second vector comprises a polynucleotide encoding the C-extein, optionally wherein the second vector further comprises a polynucleotide encoding a pegRNA and a polynucleotide encoding a ngRNA or a polynucleotide encoding a second pegRNA.

Embodiment 132. The one or more vectors of any one of embodiments 129-131, wherein the one or more vectors are AAV vectors.

Embodiment 133. One or more AAV particles comprising the one or more polynucleotides of any one of embodiments 123-126 or the one or more vectors of any one of embodiments 129-132.

Embodiment 134. The one or more AAV particles of embodiment 133, wherein the AAV particles comprise AAV1, AAV2, AAV3, AAV4, AAV5, AAV6, AAV7, AAV8, or AAV9.

Embodiment 135. The one or more AAV particles of embodiment 133 or 134, wherein the AAV particles comprise AAV9.

Embodiment 136. The one or more AAV particles of any one of embodiments 133-135, comprising a first AAV particle and a second AAV particle, wherein the first AAV particle comprises a polynucleotide comprising the structure 5′-[inverted terminal repeat (ITR) sequence]-[promoter]-[Cas9 N-terminal fragment]-[N-intein]-[terminator sequence]-[ITR sequence]-3′, and wherein the second AAV particle comprises a polynucleotide comprising the structure 5′-[ITR sequence]-[promoter]-[C-intein]-[Cas9 C-terminal fragment]-[reverse transcriptase]-[terminator sequence]-[optional nicking gRNA]-[pegRNA]-[ITR]-3′.

Embodiment 137. The one or more polynucleotides of embodiment 126 comprising one or more polynucleotides encoding the prime editor, wherein the one or more polynucleotides encoding the prime editor comprise mRNA.

Embodiment 138. A cell comprising a reverse transcriptase variant of any one of embodiments 1-22 or 92, a Cas9 variant of any one of embodiments 23-52 or 93, a prime editor of any one of embodiments 53-89, a fusion protein of embodiment 89 or 90, a complex of embodiment 99, the prime editing system of any one of embodiments 95-98 or 100-122, the one or more polynucleotides of any one of embodiments 123-126 or 137, the one or more vectors of any one of embodiments 127-132, or the one or more AAV particles of any one of embodiments 133-136.

Embodiment 139. A pharmaceutical composition comprising a reverse transcriptase variant of any one of embodiments 1-22 or 92, a Cas9 variant of any one of embodiments 23-52 or 93, a prime editor of any one of embodiments 53-89, a fusion protein of embodiment 89 or 90, a complex of embodiment 99, the prime editing system of any one of embodiments 95-98 or 100-122, the one or more polynucleotides of any one of embodiments 123-126 or 137, the one or more vectors of any one of embodiments 127-132, or the one or more AAV particles of any one of embodiments 133-136.

Embodiment 140. A method for editing a nucleic acid molecule by prime editing comprising contacting a nucleic acid molecule with a prime editor of any one of embodiments 53-89, a fusion protein of embodiment 89 or 90, a complex of embodiment 99, the prime editing system of any one of embodiments 95-98 or 100-122, the one or more polynucleotides of any one of embodiments 123-126 or 137, the one or more vectors of any one of embodiments 127-132, or the one or more AAV particles of any one of embodiments 133-136, thereby installing one or more modifications to the nucleic acid molecule at a target site.

Embodiment 141. A method for simultaneously editing both strands of a double-stranded nucleic acid molecule at a target site to be edited comprising contacting the double-stranded nucleic acid molecule with:
(a) a prime editor of any one of embodiments 53-89, or a polynucleotide encoding the prime editor;
(b) a first prime editing guide RNA (first pegRNA), or a polynucleotide encoding the first pegRNA that comprises
(i) a first spacer sequence that binds to a first binding site on a first strand of the double-stranded DNA sequence upstream of the target site relative to the second strand,
(ii) a first gRNA core that is capable of complexing with the prime editor, and
(iii) a first DNA synthesis template that encodes a first single-stranded DNA sequence,
and
(c) a second prime editing guide RNA (second pegRNA), or a polynucleotide encoding the second pegRNA, that comprises
(i) a second spacer sequence that binds to a second binding site on a second strand of the double-stranded DNA sequence downstream of the target site relative to the second strand;
(ii) a second gRNA core that is capable of complexing with the prime editor, and
(iii) a second DNA synthesis template that encodes a second single-stranded DNA sequence.

Embodiment 142. A method for simultaneously editing both strands of a double-stranded nucleic acid molecule at a target site to be edited comprising contacting the double-stranded nucleic acid molecule with the prime editing system of any one of embodiments 108-112, the one or more polynucleotides of embodiment 126 encoding the prime editing system, or the one or more vectors of embodiments 129-132 comprising the one or more polynucleotides.

Embodiment 143. The method of embodiment 88 or 89, wherein the method further comprises contacting the nucleic acid molecule with one or more second strand nicking gRNAs.

Embodiment 144. The method of any one of embodiments 140-143, wherein the method comprises installing a recombinase recognition site in the nucleic acid molecule.

Embodiment 145. The method of any one of embodiments 140-144, wherein the contacting is in vitro.

Embodiment 146. The method of any one of embodiments 140-144, wherein the contacting is ex vivo in a cell, optionally wherein the cell is a non-dividing cell.

Embodiment 147. The method of embodiment 146, wherein the cell is a nervous system cell.

Embodiment 148. The method of any one of embodiments 140-144, wherein the contacting is in vivo.

Embodiment 149. The method of embodiment 148, wherein the contacting is in a central nerve system.

Embodiment 150. A cell generated by the method of embodiment 146 or 147.

Embodiment 151. A kit comprising a reverse transcriptase variant of any one of embodiments 1-22 or 92, a Cas9 variant of any one of embodiments 23-52 or 93, a prime editor of any one of embodiments 53-89, a fusion protein of embodiment 89 or 90, a complex of embodiment 99, the prime editing system of any one of embodiments 95-98 or 100-122, the one or more polynucleotides of any one of embodiments 123-126 or 137, the one or more vectors of any one of embodiments 127-132, or the one or more AAV particles of any one of embodiments 133-136, or the cell of embodiment 138 or 150.

Embodiment 152. Use of a reverse transcriptase variant of any one of embodiments 1-22 or 92, a Cas9 variant of any one of embodiments 23-52 or 93, a prime editor of any one of embodiments 53-89, a fusion protein of embodiment 89 or 90, a complex of embodiment 99, the prime editing system of any one of embodiments 95-98 or 100-122, the one or more polynucleotides of any one of embodiments 123-126 or 137, the one or more vectors of any one of embodiments 127-132, or the one or more AAV particles of any one of embodiments 133-136, or the cell of embodiment 138 or 150 in the manufacture of a medicament.

Embodiment 153. The reverse transcriptase variant of any one of embodiments 1-22 or 92, the Cas9 variant of any one of embodiments 23-52 or 93, the prime editor of any one of embodiments 53-89, the fusion protein of embodiment 89 or 90, a complex of embodiment 99, the prime editing system of any one of embodiments 95-98 or 100-122, the one or more polynucleotides of any one of embodiments 123-126 or 137, the one or more vectors of any one of embodiments 127-132, the one or more AAV particles of any one of embodiments 133-136, the cell of embodiment 138 or 150 for use in medicine.

Embodiment 154. A system of polynucleotides for phage-assisted continuous and non-continuous evolution of prime editors comprising:
i) a first polynucleotide encoding a pegRNA and the gIII gene;
ii) a second polynucleotide encoding a Cas9 protein fused to an N-intein;
iii) a third polynucleotide encoding an RNA polymerase;
iv) a fourth polynucleotide encoding proteins capable of mutagenizing a phage, optionally wherein the fourth polynucleotide comprises the MP6 plasmid; and
v) a fifth polynucleotide encoding a reverse transcriptase fused to a C-intein.

Embodiment 155. A system of polynucleotides for phage-assisted continuous and non-continuous evolution of prime editors comprising:
i) a first polynucleotide encoding a pegRNA and the gIII gene;
ii) a second polynucleotide encoding a prime editor;
iii) a third polynucleotide encoding an RNA polymerase; and
iv) a fourth polynucleotide encoding proteins capable of mutagenizing a phage, optionally wherein the fourth polynucleotide comprises the MP6 plasmid.

### REFERENCES

1. Anzalone, A. V., Randolph, P. B., Davis, J. R., Sousa, A. A., Koblan, L. W., Levy, J. M., Chen, P. J., Wilson, C., Newby, G. A., Raguram, A., et al. (2019). Search-and-replace genome editing without double-strand breaks or donor DNA. Nature 576, 149-157. 10.1038/s41586-019-1711-4.
2. Landrum, M. J., Lee, J. M., Benson, M., Brown, G. R., Chao, C., Chitipiralla, S., Gu, B., Hart, J., Hoffman, D., Jang, W., et al. (2018). ClinVar: improving access to variant interpretations and supporting evidence. Nucleic Acids Research 46, D1062-D1067. 10.1093/nar/gkx1153.
3. Chen, P. J., Hussmann, J. A., Yan, J., Knipping, F., Ravisankar, P., Chen, P.-F., Chen, C., Nelson, J. W., Newby, G. A., Sahin, M., et al. (2021). Enhanced prime editing systems by manipulating cellular determinants of editing outcomes. Cell 184, 5635-5652.e29. 10.1016/j.cell.2021.09.018.
4. Liu, B., Dong, X., Cheng, H., Zheng, C., Chen, Z., Rodriguez, T. C., Liang, S.-Q., Xue, W., and Sontheimer, E. J. (2022). A split prime editor with untethered reverse transcriptase and circular RNA template. Nat Biotechnol. 40, 1388-1393 10.1038/s41587-022-01255-9.
5. Nelson, J. W., Randolph, P. B., Shen, S. P., Everette, K. A., Chen, P. J., Anzalone, A. V., An, M., Newby, G. A., Chen, J. C., Hsu, A., et al. (2021). Engineered pegRNAs improve prime editing efficiency. Nat Biotechnol. 40, 402-410 10.1038/s41587-021-01039-7.
6. Zhang, G., Liu, Y., Huang, S., Qu, S., Cheng, D., Yao, Y., Ji, Q., Wang, X., Huang, X., and Liu, J. (2022). Enhancement of prime editing via xrRNA motif-joined pegRNA. Nat Commun 13, 1856. 10.1038/s41467-022-29507-x.
7. Velimirovic, M., Zanetti, L. C., Shen, M. W., Fife, J. D., Lin, L., Cha, M., Akinci, E., Barnum, D., Yu, T., and Sherwood, R. I. (2022). Peptide fusion improves prime editing efficiency. Nat Commun 13, 3512. 10.1038/s41467-022-31270-y.
8. Zong, Y., Liu, Y., Xue, C., Li, B., Li, X., Wang, Y., Li, J., Liu, G., Huang, X., Cao, X., et al. (2022). An engineered prime editor with enhanced editing efficiency in plants. Nat Biotechnol. 40, 1394-1402 10.1038/s41587-022-01254-w.
9. Ferreira da Silva, J., Oliveira, G. P., Arasa-Verge, E. A., Kagiou, C., Moretton, A., Timelthaler, G., Jiricny, J., and Loizou, J. I. (2022). Prime editing efficiency and fidelity are enhanced in the absence of mismatch repair. Nat Commun 13, 760. 10.1038/s41467-022-28442-1.
10. Anzalone, A. V., Gao, X. D., Podracky, C. J., Nelson, A. T., Koblan, L. W., Raguram, A., Levy, J. M., Mercer, J. A. M., and Liu, D. R. (2022). Programmable deletion, replacement, integration and inversion of large DNA sequences with twin prime editing. Nat Biotechnol 40, 731-740. 10.1038/s41587-021-01133-w.
11. Choi, J., Chen, W., Suiter, C. C., Lee, C., Chardon, F. M., Yang, W., Leith, A., Daza, R. M., Martin, B., and Shendure, J. (2021). Precise genomic deletions using paired prime editing. Nat Biotechnol. 40, 218-226 10.1038/s41587-021-01025-z.
12. Jiang, T., Zhang, X.-O., Weng, Z., and Xue, W. (2021). Deletion and replacement of long genomic sequences using prime editing. Nat Biotechnol. 40, 227-234 10.1038/s41587-021-01026-y.
13. Lin, Q., Jin, S., Zong, Y., Yu, H., Zhu, Z., Liu, G., Kou, L., Wang, Y., Qiu, J.-L., Li, J., et al. (2021). High-efficiency prime editing with optimized, paired pegRNAs in plants. Nat Biotechnol 39, 923-927. 10.1038/s41587-021-00868-w.
14. Tao, R., Wang, Y., Jiao, Y., Hu, Y., Li, L., Jiang, L., Zhou, L., Qu, J., Chen, Q., and Yao, S. (2022). Bi-PE: bi-directional priming improves CRISPR/Cas9 prime editing in mammalian cells. Nucleic Acids Research 50, 6423-6434. 10.1093/nar/gkac506.
15. Wang, J. (2022). Efficient targeted insertion of large DNA fragments without DNA donors. Nature Methods 19, 25. 10.1038/s41592-022-01399-1
16. Zhuang, Y., Liu, J., Wu, H., Zhu, Q., Yan, Y., Meng, H., Chen, P. R., and Yi, C. (2021). Increasing the efficiency and precision of prime editing with guide RNA pairs. Nat Chem Biol. 18, 29-37 10.1038/s41589-021-00889-1.
17. Yarnall, M. T. N., Ioannidi, E. I., Schmitt-Ulms, C., Krajeski, R. N., Lim, J., Villiger, L., Zhou, W., Jiang, K., Garushyants, S. K., Roberts, N., et al. (2022). Drag-and-drop genome insertion of large sequences without double-strand DNA cleavage using CRISPR-directed integrases. 41, 500-512 Nat Biotechnol. 10.1038/s41587-022-01527-4.
18. Arezi, B., and Hogrefe, H. (2009). Novel mutations in Moloney Murine Leukemia Virus reverse transcriptase increase thermostability through tighter binding to template-primer. Nucleic Acids Research 37, 473-481. 10.1093/nar/gkn952.
19. Baranauskas, A., Paliksa, S., Alzbutas, G., Vaitkevicius, M., Lubiene, J., Letukiene, V., Burinskas, S., Sasnauskas, G., and Skirgaila, R. (2012). Generation and characterization of new highly thermostable and processive M-MuLV reverse transcriptase variants. Protein Engineering Design and Selection 25, 657-668. 10.1093/protein/gzs034.
20. Gerard, G. F. (2002). The role of template-primer in protection of reverse transcriptase from thermal inactivation. Nucleic Acids Research 30, 3118-3129. 10.1093/nar/gkf417.
21. Kotewicz, M. L., Sampson, C. M., D'Alessio, J. M., and Gerard, G. F. (1988).
Isolation of cloned Moloney murine leukemia virus reverse transcriptase lacking ribonuclease H activity. Nucl Acids Res 16, 265-277. 10.1093/nar/16.1.265.
22. Anzalone, A. V., Koblan, L. W., and Liu, D. R. (2020). Genome editing with CRISPR-Cas nucleases, base editors, transposases and prime editors. Nat Biotechnol 38, 824-844. 10.1038/s41587-020-0561-9.
23. Grünewald, J., Miller, B. R., Szalay, R. N., Cabeceiras, P. K., Woodilla, C. J., Holtz, E. J. B., Petri, K., and Joung, J. K. (2022). Engineered CRISPR prime editors with compact, untethered reverse transcriptases. Nat Biotechnol. 41, 337-343 10.1038/s41587-022-01473-1.
24. Lin, Q., Zong, Y., Xue, C., Wang, S., Jin, S., Zhu, Z., Wang, Y., Anzalone, A. V., Raguram, A., Doman, J. L., et al. (2020). Prime genome editing in rice and wheat. Nat Biotechnol 38, 582-585. 10.1038/s41587-020-0455-x.
25. Zong, Y., Liu, Y., Xue, C., Li, B., Li, X., Wang, Y., Li, J., Liu, G., Huang, X., Cao, X., et al. (2022). Author Correction: An engineered prime editor with enhanced editing efficiency in plants. Nat Biotechnol. 40, 1394-1402 10.1038/s41587-022-01308-z.
26. Esvelt, K. M., Carlson, J. C., and Liu, D. R. (2011). A system for the continuous directed evolution of biomolecules. Nature 472, 499-503. 10.1038/nature09929.
27. Millman, A., Bernheim, A., Stokar-Avihail, A., Fedorenko, T., Voichek, M., Leavitt, A., Oppenheimer-Shaanan, Y., and Sorek, R. (2020). Bacterial Retrons Function In Anti-Phage Defense. Cell 183, 1551-1561.e12. 10.1016/j.cell.2020.09.065.
28. Kirshenboim, N., Hayouka, Z., Friedler, A., and Hizi, A. (2007). Expression and characterization of a novel reverse transcriptase of the LTR retrotransposon Tf1. Virology 366, 263-276. 10.1016/j.virol.2007.04.002.
29. Davis, J., Banskota, S., Levy, J. M., Newby, G. A., Wang, X., Anzalone, A. V., Nelson, A. T., Chen, P. J., An, M., Roh, H., et al. (2023). Efficient AAV-mediated in vivo prime editing in multiple organs. Nat Biotechnol, available online. https://doi.org/10.1038/s41587-023-01758-z
30. Böck, D., Rothgangl, T., Villiger, L., Schmidheini, L., Mathis, N., Ioannidi, E., Kreutzer, S., Kontarakis, Z., Rimann, N., Grisch-Chan, H. M., et al. (2021). In vivo prime editing of a metabolic liver disease in mice. Science Translational Medicine 14, 636. 10.1126/scitranslmed.ab19238.
31. Zhi, S., Chen, Y., Wu, G., Wen, J., Wu, J., Liu, Q., Li, Y., Kang, R., Hu, S., Wang, J., et al. (2021). Dual-AAV delivering split prime editor system for in vivo genome editing. Molecular Therapy 30, 283-294. https://www.sciencedirect.com/science/article/abs/pii/S1525001621003658.
32. Wang, Y., Guan, Z., Wang, C., Nie, Y., Chen, Y., Qian, Z., Cui, Y., Xu, H., Wang, Q., Zhao, F., et al. (2022). Cryo-EM structures of Escherichia coli Ec86 retron complexes reveal architecture and defence mechanism. Nat Microbiol 7, 1480-1489. 10.1038/s41564-022-01197-7.
33. Jumper, J., Evans, R., Pritzel, A., Green, T., Figurnov, M., Ronneberger, O., Tunyasuvunakool, K., Bates, R., Žídek, A., Potapenko, A., et al. (2021). Highly accurate protein structure prediction with AlphaFold. Nature 596, 583-589. 10.1038/s41586-021-03819-2.
34. Nowak, E., Potrzebowski, W., Konarev, P. V., Rausch, J. W., Bona, M. K., Svergun, D. I., Bujnicki, J. M., Le Grice, S. F. J., and Nowotny, M. (2013). Structural analysis of monomeric retroviral reverse transcriptase in complex with an RNA/DNA hybrid. Nucleic Acids Research 41, 3874-3887. 10.1093/nar/gkt053.
35. Roth, T. B., Woolston, B. M., Stephanopoulos, G., and Liu, D. R. (2019). Phage-Assisted Evolution of Bacillus methanolicus Methanol Dehydrogenase 2. ACS Synth. Biol. 8, 796-806. 10.1021/acssynbio.8b00481.
36. Badran, A. H., and Liu, D. R. (2015). Development of potent in vivo mutagenesis plasmids with broad mutational spectra. Nat Commun 6, 8425. 10.1038/ncomms9425.
37. Doman, J. L., Sousa, A. A., Randolph, P. B., Chen, P. J., and Liu, D. R. (2022).
Designing and executing prime editing experiments in mammalian cells. Nat Protoc 17, 2431-2468. 10.1038/s41596-022-00724-4.
38. Koeppel, J., Weller, J., Peets, E. M., Pallaseni, A., Kuzmin, I., Raudvere, U., Peterson, H., Liberante, F. G., and Parts, L. (2023). Prediction of prime editing insertion efficiencies using sequence features and DNA repair determinants. Nat Biotechnol., available online. 10.1038/s41587-023-01678-y.
39. Kim, H. K., Yu, G., Park, J., Min, S., Lee, S., Yoon, S., and Kim, H. H. (2021). Predicting the efficiency of prime editing guide RNAs in human cells. Nat Biotechnol 39, 198-206. 10.1038/s41587-020-0677-y.
40. Mathis, N., Allam, A., Kissling, L., Marquart, K. F., Schmidheini, L., Solari, C., Balizs, Z., Krauthammer, M., and Schwank, G. (2023). Predicting prime editing efficiency and product purity by deep learning. Nat Biotechnol., available online. 10.1038/s41587-022-01613-7.
41. Yu, G., Kim, H. K., Park, J., Kwak, H., Cheong, Y., Kim, D., Kim, J., Kim, J., and Kim, H. H. (2023). Prediction of efficiencies for diverse prime editing systems in multiple cell types. Cell, epub ahead of print, 10.1016/j.cell.2023.03.034.
42. Dickinson, B. C., Leconte, A. M., Allen, B., Esvelt, K. M., and Liu, D. R. (2013). Experimental interrogation of the path dependence and stochasticity of protein evolution using phage-assisted continuous evolution. Proc. Natl. Acad. Sci. U.S.A. 110, 9007-9012. 10.1073/pnas.1220670110.
43. Stamos, J. L., Lentzsch, A. M., and Lambowitz, A. M. (2017). Structure of a Thermostable Group II Intron Reverse Transcriptase with Template-Primer and Its Functional and Evolutionary Implications. Molecular Cell 68, 926-939.e4. 10.1016/j.molcel.2017.10.024.
44. Mohr, S., Ghanem, E., Smith, W., Sheeter, D., Qin, Y., King, O., Polioudakis, D., Iyer, V. R., Hunicke-Smith, S., Swamy, S., et al. (2013). Thermostable group II intron reverse transcriptase fusion proteins and their use in cDNA synthesis and next-generation RNA sequencing. RNA 19, 958-970. 10.1261/rna.039743.113.
45. Flotte, T. R., Cataltepe, O., Puri, A., Batista, A. R., Moser, R., McKenna-Yasek, D., Douthwright, C., Gernoux, G., Blackwood, M., Mueller, C., et al. (2022). AAV gene therapy for Tay-Sachs disease. Nat Med 28, 251-259. 10.1038/s41591-021-01664-4.
46. Sharma, P. L., Nurpeisov, V., and Schinazi, R. F. (2005). Retrovirus Reverse Transcriptases Containing a Modified YXDD Motif. Antivir Chem Chemother 16, 169-182. 10.1177/095632020501600303.
47. Zadeh, J. N., Steenberg, C. D., Bois, J. S., Wolfe, B. R., Pierce, M. B., Khan, A. R., Dirks, R. M., and Pierce, N. A. (2011). NUPACK: Analysis and design of nucleic acid systems. J. Comput. Chem. 32, 170-173. 10.1002/jcc.21596.
48. Telesnitsky, A., and Goff, S. P. (1993). RNase H domain mutations affect the interaction between Moloney murine leukemia virus reverse transcriptase and its primer-template. Proc. Natl. Acad. Sci. U.S.A. 90, 1276-1280. 10.1073/pnas.90.4.1276.
49. Izsvak, Z. (2009). Efficient stable gene transfer into human cells by the Sleeping Beauty transposon vectors. Methods 49, 287-297. 10.1016/j.ymeth.2009.07.001.
50. Anders, C., Niewoehner, O., Duerst, A., and Jinek, M. (2014). Structural basis of PAM-dependent target DNA recognition by the Cas9 endonuclease. Nature 513, 569-573. 10.1038/nature13579
51. Chen, J. S., Dagdas, Y. S., Kleinstiver, B. P., Welch, M. M., Sousa, A. A., Harrington, L. B., Sternberg, S. H., Joung, J. K., Yildiz, A., and Doudna, J. A. (2017). Enhanced proofreading governs CRISPR-Cas9 targeting accuracy. Nature 550, 407-410. 10.1038/nature24268.
52. Lapinaite, A., Knott, G. J., Palumbo, C. M., Lin-Shiao, E., Richter, M. F., Zhao, K. T., Beal, P. A., Liu, D. R., and Doudna, J. A. (2020). DNA capture by a CRISPR-Cas9-guided adenine base editor. Science 369, 566-571. 10.1126/science.abb1390.
53. Nishimasu, H., Ran, F. A., Hsu, P., Konermann, S., Shehata, S., Dohmae, N., Ishitani, R., Zhang, F., and Nureki, O. (2014). Crystal Structure of Cas9 in Complex with Guide RNA and Target DNA. Cell 156, 935-949. 10.1093/nar/gkt1074
54. Slaymaker, I. M., Gao, L., Zetsche, B., Scott, D. A., Yan, W. X., and Zhang, F. (2016). Rationally engineered Cas9 nucleases with improved specificity. Science 351, 84-88. 10.1126/science.aad5227.
55. Qi, L. S., Larson, M. H., Gilbert, L. A., Doudna, J. A., Weissman, J. S., Arkin, A. P., and Lim, W. A. (2013). Repurposing CRISPR as an RNA-Guided Platform for Sequence-Specific Control of Gene Expression. Cell 152, 1173-1183. 10.1016/j.cell.2013.02.022.
56. Jiang, F., and Doudna, J. A. (2017). CRISPR-Cas9 Structures and Mechanisms. Annu. Rev. Biophys. 46, 505-529. 10.1146/annurev-biophys-062215-010822.
57. Zeng, Y., Cui, Y., Zhang, Y., Zhang, Y., Liang, M., Chen, H., Lan, J., Song, G., and Lou, J. (2018). The initiation, propagation and dynamics of CRISPR-SpyCas9 R-loop complex. Nucleic Acids Research 46, 350-361. 10.1093/nar/gkx1117.
58. Liu, D. R., Anzalone, A. V., Newby, G. A., and Everette, K. A. (2022). Methods and compositions for prime editing nucleotide sequences. U.S. Pat. No. 1,447,770.
59. Mendell, J. R., Al-Zaidy, S. A., Lehman, K. J., McColly, M., Lowes, L. P., Alfano, L. N., Reash, N. F., Iammarino, M. A., Church, K. R., Kleyn, A., et al. (2021). Five-Year Extension Results of the Phase 1 START Trial of Onasemnogene Abeparvovec in Spinal Muscular Atrophy. JAMA Neurol 78, 834. 10.1001/jamaneurol.2021.1272.
60. Lazzarotto, C. R., Malinin, N. L., Li, Y., Zhang, R., Yang, Y., Lee, G., Cowley, E., He, Y., Lan, X., Jividen, K., et al. (2020). CHANGE-seq reveals genetic and epigenetic effects on CRISPR-Cas9 genome-wide activity. Nat Biotechnol 38, 1317-1327. 10.1038/s41587-020-0555-7.
61. Huang, T. P., Heins, Z. J., Miller, S. M., Wong, B. G., Balivada, P. A., Wang, T., Khalil, A. S., and Liu, D. R. (2022). High-throughput continuous evolution of compact Cas9 variants targeting single-nucleotide-pyrimidine PAMs. Nat Biotechnol. 41, 96-107 10.1038/s41587-022-01410-2.
62. Liu, P., Liang, S.-Q., Zheng, C., Mintzer, E., Zhao, Y. G., Ponnienselvan, K., Mir, A., Sontheimer, E. J., Gao, G., Flotte, T. R., et al. (2021). Improved prime editors enable pathogenic allele correction and cancer modelling in adult mice. Nat Commun 12, 2121. 10.1038/s41467-021-22295-w.
63. Banskota, S., Raguram, A., Suh, S., Du, S. W., Davis, J. R., Choi, E. H., Wang, X., Nielsen, S. C., Newby, G. A., Randolph, P. B., et al. (2022). Engineered virus-like particles for efficient in vivo delivery of therapeutic proteins. Cell 185, 250-265.
64. Nishiyama, J., Mikuni, T., and Yasuda, R. (2017). Virus-Mediated Genome Editing via Homology-Directed Repair in Mitotic and Postmitotic Cells in Mammalian Brain. Neuron 96, 755-768.e5. 10.1016/j.neuron.2017.10.004.
65. Suzuki, K., Tsunekawa, Y., Hernandez-Benitez, R., Wu, J., Zhu, J., Kim, E. J., Hatanaka, F., Yamamoto, M., Araoka, T., Li, Z., et al. (2016). In vivo genome editing via CRISPR/Cas9 mediated homology-independent targeted integration. Nature 540, 144-149. 10.1038/nature20565.
66. Clement, K., Rees, H., Canver, M., Gehrke, J., Farouni, R., Hsu, J., Cole, M., Liu, D., Joung, K., Bauer, D., et al. (2019). CRISPResso2 provides accurate and rapid genome editing sequence analysis. Nat Biotechnol 37, 215-216. 10.1038/s41587-019-0043-0.
67. Clement, K., Farouni, R., Bauer, D. E., and Pinello, L. (2018). AmpUMI: design and analysis of unique molecular identifiers for deep amplicon sequencing. Bioinformatics 34, i202-i210. 10.1093/bioinformatics/bty264.
68. Mok, B. Y., Kotrys, A. V., Raguram, A., Huang, T. P., Mootha, V. K., and Liu, D. R. (2022). CRISPR-free base editors with enhanced activity and expanded targeting scope in mitochondrial and nuclear DNA. Nat Biotechnol 40, 1378-1387. 10.1038/s41587-022-01256-8.
69. Richter, M. F., Zhao, K. T., Eton, E., Lapinaite, A., Newby, G. A., Thuronyi, B. W., Wilson, C., Koblan, L. W., Zeng, J., Bauer, D. E., et al. (2020). Phage-assisted evolution of an adenine base editor with improved Cas domain compatibility and activity. Nat Biotechnol 38, 883-891. 10.1038/s41587-020-0453-z.
70. Thuronyi, B. W., Koblan, L. W., Levy, J. M., Yeh, W.-H., Zheng, C., Newby, G. A., Wilson, C., Bhaumik, M., Shubina-Oleinik, O., Holt, J. R., et al. (2019). Continuous evolution of base editors with expanded target compatibility and improved activity. Nat Biotechnol 37, 1070-1079. 10.1038/s41587-019-0193-0.
71. Engler, C., Kandzia, R., and Marillonnet, S. (2008). A One Pot, One Step, Precision Cloning Method with High Throughput Capability. PLoS ONE 3, e3647. 10.1371/journal.pone.0003647.
72. Hubbard, B. P., Badran, A. H., Zuris, J. A., Guilinger, J. P., Davis, K. M., Chen, L., Tsai, S. Q., Sander, J. D., Joung, J. K., and Liu, D. R. (2015). Continuous directed evolution of DNA-binding proteins to improve TALEN specificity. Nat Methods 12, 939-942. 10.1038/nmeth.3515.
73. Miller, S. M., Wang, T., and Liu, D. R. (2020). Phage-assisted continuous and non-continuous evolution. Nat Protoc 15, 4101-4127. 10.1038/s41596-020-00410-3.
74. Levy, J. M., Yeh, W.-H., Pendse, N., Davis, J. R., Hennessey, E., Butcher, R., Koblan, L. W., Comander, J., Liu, Q., and Liu, D. R. (2020). Cytosine and adenine base editing of the brain, liver, retina, heart and skeletal muscle of mice via adeno-associated viruses. Nat Biomed Eng 4, 97-110. 10.1038/s41551-019-0501-5.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A reverse transcriptase variant having at least 80%, at least 85%, at least 90%, at least 95%, at least 96%, at least 97%, at least 98%, or at least 99% sequence identity with SEQ ID NO: 1, wherein the reverse transcriptase variant comprises amino acid substitutions at positions 70, 72, 87, 102, 106, 118, 128, 158, 269, 363, 413, and 492 relative to SEQ ID NO: 1, or corresponding substitutions in a homologous sequence.

2. The reverse transcriptase variant of claim 1, wherein the amino acid substitutions comprise P70T, G72V, S87G, M102I, K106R, K118R, I128V, L158Q, F269L, A363V, K413E, and S492N relative to SEQ ID NO: 1.

3. The reverse transcriptase variant of claim 1 or 2 further comprising amino acid substitutions at positions 188, 260, 297, and 288 relative to SEQ ID NO: 1.

4. The reverse transcriptase variant of claim 3, wherein the amino acid substitutions comprise S188K, 1260L, S297Q, and R288Q relative to SEQ ID NO: 1.

5. A reverse transcriptase variant having at least 80%, at least 85%, at least 90%, at least 95%, at least 96%, at least 97%, at least 98%, or at least 99% sequence identity with SEQ ID NO: 30, wherein the reverse transcriptase variant comprises amino acid substitutions at positions 128 and 200 relative to SEQ ID NO: 30, or corresponding substitutions in a homologous sequence.

6. The reverse transcriptase variant of claim 5, wherein the amino acid substitutions comprise T128N and D200C relative to SEQ ID NO: 30.

7. The reverse transcriptase variant of claim 5 or 6 further comprising amino acid substitutions at positions 223, 306, 313, and 330 relative to SEQ ID NO: 30.

8. The reverse transcriptase variant of claim 7, wherein the amino acid substitutions comprise V223Y, T306K, W313F, and T330P relative to SEQ ID NO: 30.

9. A reverse transcriptase variant having at least 80%, at least 85%, at least 90%, at least 95%, at least 96%, at least 97%, at least 98%, or at least 99% sequence identity with SEQ ID NO: 30, wherein the reverse transcriptase variant comprises the amino acid substitutions T128N and V223M; T128N and V223Y; T128F and V223M; or D200C and V223M relative to SEQ ID NO: 30, or corresponding substitutions in a homologous sequence, optionally wherein the reverse transcriptase variant further comprises one or more of the amino acid substitutions D200N, T306K, W313F, T330P, and L603W relative to SEQ ID NO: 30.

10. A reverse transcriptase variant having at least 80%, at least 85%, at least 90%, at least 95%, at least 96%, at least 97%, at least 98%, or at least 99% sequence identity with SEQ ID NO: 30, wherein the reverse transcriptase variant comprises amino acid substitutions at positions 128, 129, 196, 200, and 223 relative to SEQ ID NO: 30, or corresponding substitutions in a homologous sequence.

11. The reverse transcriptase variant of claim 10, wherein the amino acid substitutions comprise T128N; V129A or V129G; P196S, P196T, or P196F; N200S or N200Y; and V223A, V223M, V223L, or V223E relative to SEQ ID NO: 30, optionally wherein the reverse transcriptase variant further comprises one or more of the amino acid substitutions D200N, T306K, W313F, T330P, and L603W relative to SEQ ID NO: 30.

12. The reverse transcriptase variant of any one of claims 5-11, wherein the reverse transcriptase variant comprises a C-terminal truncation of part or all of the RNaseH domain of SEQ ID NO: 30.

13. The reverse transcriptase variant of claim 12, wherein the C-terminal truncation is between amino acid positions D497 and I498 of SEQ ID NO: 30.

14. A Cas9 variant having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 775 and 918 relative to SEQ ID NO: 2, or corresponding substitutions in a homologous sequence.

15. The Cas9 variant of claim 14, wherein the amino acid substitutions comprise K775R and K918A relative to SEQ ID NO: 2.

16. A Cas9 variant having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 99, 471, 632, 645, and 721 relative to SEQ ID NO: 2, or corresponding substitutions in a homologous sequence.

17. The Cas9 variant of claim 16, wherein the amino acid substitutions comprise H99R, E471K, I632V, D645N, and H721Y relative to SEQ ID NO: 2.

18. The Cas9 variant of claim 16 or 17 further comprising an amino acid substitution at position 654 relative to SEQ ID NO: 2.

19. The Cas9 variant of claim 18, wherein the amino acid substitution comprises R654C relative to SEQ ID NO: 2.

20. The Cas9 variant of any one of claims 16-19 further comprising an amino acid substitution at position 918 relative to SEQ ID NO: 2.

21. The Cas9 variant of claim 20, wherein the amino acid substitution comprises K918A relative to SEQ ID NO: 2.

22. A Cas9 variant having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 99, 471, and 632 relative to SEQ ID NO: 2, or corresponding substitutions in a homologous sequence.

23. The Cas9 variant of claim 22, wherein the amino acid substitutions comprise H99R, E471K, and I632V relative to SEQ ID NO: 2.

24. The Cas9 variant of claim 22 or 23 further comprising an amino acid substitution at position 721 relative to SEQ ID NO: 2.

25. The Cas9 variant of claim 24, wherein the amino acid substitution is H721Y relative to SEQ ID NO: 2.

26. A Cas9 variant having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 471 and 918 relative to SEQ ID NO: 2, or corresponding substitutions in a homologous sequence.

27. The Cas9 variant of claim 26, wherein the amino acid substitutions comprise E471K and K918A relative to SEQ ID NO: 2.

28. A Cas9 variant having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 753 and 1151 relative to SEQ ID NO: 2, or corresponding substitutions in a homologous sequence.

29. The Cas9 variant of claim 28, wherein the amino acid substitutions comprise R753G and K1151E relative to SEQ ID NO: 2.

30. A Cas9 variant having at least 80%, at least 85%, at least 90%, at least 95%, at least 96%, at least 97%, at least 98%, or at least 99% sequence identity with SEQ ID NO: 2, wherein the Cas9 variant comprises one or more amino acid substitutions at positions selected from the group consisting of 260, 298, 395, 769, 778, 1014, 1034, 1100, 1106, 1138, 1152, and 1320 relative to SEQ ID NO: 2, or corresponding substitutions in a homologous sequence.

31. The Cas9 variant of claim 30, wherein the one or more amino acid substitutions are selected from the group consisting of E260K, D298N, R395C, T769P, R778Q, K1014E, A1034E, V1100I, S1106F, T1138A, G1152E, and A1320T.

32. The Cas9 variant of claim 30 or 31 further comprising one or more additional amino acid substitutions at positions selected from the group consisting of 102, 753, 804, and 1003 relative to SEQ ID NO: 2.

33. The Cas9 variant of claim 32, wherein the one or more additional amino acid substitutions are selected from the group consisting of E102K, R753G, T804A, and K1003R.

34. The Cas9 variant of any one of claims 30-33 comprising amino acid substitutions at any one of the groups of positions:
102, 395, 753, 778, and 1100;
753, 769, 1034, and 1320;
298, 753, 1034, and 1138;
102, 260, 395, 753, 778, 804, 1003, 1100, 1106, and 1152; or
102, 260, 395, 753, 778, 804, 1003, 1014, 1100, 1106, and 1152;
relative to SEQ ID NO: 2.

35. The Cas9 variant of any one of claims 30-34 comprising amino acid substitutions at any one of the groups of positions:
E102K, R395C, R753G, R778Q, and V1100I;
R753G, T769P, A1034E, and A1320T;
D298N, R753G, A1034E, and T1138A;
E102K, E260K, R395C, R753C, R778Q, T804A, K1003R, V1100I, S1106F, and G1152E; or
E102K, E260K, R395C, R753G, R778Q, T804A, K1003R, K1014E, V1100I, S1106F, and G1152E;
relative to SEQ ID NO: 2.

36. A Cas9 variant having at least 80%, at least 85%, at least 90%, at least 95%, at least 96%, at least 97%, at least 98%, or at least 99% sequence identity with SEQ ID NO: 2, wherein the Cas9 variant comprises amino acid substitutions at positions 23 and 754 relative to SEQ ID NO:
2, or corresponding substitutions in a homologous sequence.

37. The Cas9 variant of claim 36, wherein the amino acid substitutions are D23G and H754R.

38. A prime editor comprising a reverse transcriptase variant of any one of claims 1-13 and a nucleic acid-programmable DNA-binding protein (napDNAbp).

39. The prime editor of claim 38, wherein the napDNAbp comprises a Cas9 protein.

40. The prime editor of claim 39, wherein the Cas9 protein is a Cas9 nickase.

41. The prime editor of any one of claims 38-40, wherein the napDNAbp comprises a Cas9 variant of any one of claims 14-37.

42. The prime editor of any one of claims 38-40, wherein the napDNAbp comprises a Cas9 protein of any one of SEQ ID NOs: 2, 6, 8, 9, 12-24, or 133, or an amino acid sequence at least 80%, at least 85%, at least 90%, at least 95%, at least 96%, at least 97%, at least 98%, or at least 99% identical to any one of SEQ ID NOs: 2, 6, 8, 9, 12-24, or 133.

43. The prime editor of any one of claims 38-40, wherein the napDNAbp comprises a Cas9 protein of SEQ ID NO: 133, or an amino acid sequence at least 80%, at least 85%, at least 90%, at least 95%, at least 96%, at least 97%, at least 98%, or at least 99% identical to SEQ ID NO: 133.

44. A prime editor comprising a Cas9 variant of any one of claims 14-37 and a polymerase.

45. The prime editor of claim 44, wherein the polymerase is a reverse transcriptase.

46. The prime editor of claim 45, wherein the reverse transcriptase is a reverse transcriptase variant of any one of claims 1-13.

47. The prime editor of claim 45, wherein the reverse transcriptase comprises a sequence having at least 80%, at least 85%, at least 90%, at least 95%, at least 96%, at least 97%, at least 98%, or at least 99% sequence identity with SEQ ID NO: 7, wherein the reverse transcriptase comprises amino acid substitutions at positions 60, 87, 165, 243, 267, 279, 318, and 343 relative to SEQ ID NO: 7, or corresponding positions in a homologous sequence.

48. The prime editor of claim 47, wherein the amino acid substitutions comprise E60K, K87E, E165D, D243N, R267I, E279K, K318E, and K343N relative to SEQ ID NO: 7.

49. The prime editor of any one of claims 38-48, wherein the napDNAbp and the reverse transcriptase are provided in trans or are not fused to one another.

50. The prime editor of any one of claims 38-48, wherein the napDNAbp and the reverse transcriptase are provided as a fusion protein or are fused to one another.

51. The prime editor of claim 50, wherein the napDNAbp and the reverse transcriptase are fused via a linker.

52. The prime editor of claim 51, wherein the linker comprises any one of SEQ ID NOs: 80-93.

53. The prime editor of any one of claims 38-52 further comprising a nuclear localization sequence (NLS).

54. A prime editor comprising a Cas9 protein and a reverse transcriptase variant having at least 80%, at least 85%, at least 90%, at least 95%, at least 96%, at least 97%, at least 98%, or at least 99% sequence identity with SEQ ID NO: 1, wherein the reverse transcriptase variant comprises the amino acid substitutions P70T, G72V, S87G, M102I, K106R, K118R, I128V, L158Q, F269L, A363V, K413E, and S492N relative to SEQ ID NO: 1.

55. A prime editor comprising a Cas9 protein and a reverse transcriptase variant having at least 80%, at least 85%, at least 90%, at least 95%, at least 96%, at least 97%, at least 98%, or at least 99% sequence identity with SEQ ID NO: 1, wherein the reverse transcriptase variant comprises the amino acid substitutions P70T, G72V, S87G, M102I, K106R, K118R, I128V, L158Q, S188K, I260L, F269L, R288Q, S297Q, A363V, K413E, and S492N relative to SEQ ID NO: 1.

56. A prime editor comprising a Cas9 protein and a reverse transcriptase variant having at least 80%, at least 85%, at least 90%, at least 95%, at least 96%, at least 97%, at least 98%, or at least 99% sequence identity with SEQ ID NO: 30, wherein the reverse transcriptase variant comprises the amino acid substitutions T128N, D200C, and V223Y relative to SEQ ID NO: 30.

57. The prime editor of any one of claims 54-56, wherein the Cas9 protein comprises a Cas9 variant having at least 80%, at least 85%, at least 90%, at least 95%, at least 96%, at least 97%, at least 98%, or at least 99% sequence identity with SEQ ID NO: 2, wherein the Cas9 variant comprises the amino acid substitutions K775R and K918A; H99R, E471K, I632V, D645N, H721Y, and K918A; or H99R, E471K, I632V, D645N, R654C, and H721Y relative to SEQ ID NO: 2.

58. A prime editor comprising a reverse transcriptase and a Cas9 variant having at least 80%, at least 85%, at least 90%, at least 95%, at least 96%, at least 97%, at least 98%, or at least 99% sequence identity with SEQ ID NO: 2, wherein the Cas9 variant comprises the amino acid substitutions K775R and K918A relative to SEQ ID NO: 2.

59. A prime editor comprising a reverse transcriptase and a Cas9 variant having at least 80%, at least 85%, at least 90%, at least 95%, at least 96%, at least 97%, at least 98%, or at least 99% sequence identity with SEQ ID NO: 2, wherein the Cas9 variant comprises the amino acid substitutions H99R, E471K, I632V, D645N, H721Y, and K918A relative to SEQ ID NO: 2.

60. A prime editor comprising a reverse transcriptase and a Cas9 variant having at least 80%, at least 85%, at least 90%, at least 95%, at least 96%, at least 97%, at least 98%, or at least 99% sequence identity with SEQ ID NO: 2, wherein the Cas9 variant comprises the amino acid substitutions H99R, E471K, I632V, D645N, R654C, and H721Y relative to SEQ ID NO: 2.

61. The prime editor of any one of claims 58-60, wherein the reverse transcriptase comprises a reverse transcriptase variant having at least 80%, at least 85%, at least 90%, at least 95%, at least 96%, at least 97%, at least 98%, or at least 99% sequence identity with SEQ ID NO: 1, wherein the reverse transcriptase variant comprises the amino acid substitutions P70T, G72V, S87G, M102I, K106R, K118R, I128V, L158Q, F269L, A363V, K413E, and S492N; or P70T, G72V, S87G, M102I, K106R, K118R, I128V, L158Q, S188K, 1260L, F269L, R288Q, S297Q, A363V, K413E, and S492N relative to SEQ ID NO: 1.

62. The prime editor of any one of claims 58-60, wherein the reverse transcriptase comprises a reverse transcriptase variant having at least 80%, at least 85%, at least 90%, at least 95%, at least 96%, at least 97%, at least 98%, or at least 99% sequence identity with SEQ ID NO: 30, wherein the reverse transcriptase variant comprises the amino acid substitutions T128N, D200C, and V223Y relative to SEQ ID NO: 30.

63. The prime editor of any one of claims 58-60, wherein the reverse transcriptase comprises a reverse transcriptase variant having at least 80%, at least 85%, at least 90%, at least 95%, at least 96%, at least 97%, at least 98%, or at least 99% sequence identity with SEQ ID NO: 7, wherein the reverse transcriptase comprises the amino acid substitutions E60K, K87E, E165D, D243N, R267I, E279K, K318E, and K343N relative to SEQ ID NO: 7.

64. A prime editor comprising a Cas9 variant of any one of SEQ ID NOs: 28, 48, or 49 and a reverse transcriptase variant of any one of SEQ ID NOs: 25-27 or 50, or a Cas9 variant at least 80%, at least 85%, at least 90%, at least 95%, at least 96%, at least 97%, at least 98%, or at least 99% to any one of SEQ ID NOs: 28, 48, or 49 and a reverse transcriptase variant at least 80%, at least 85%, at least 90%, at least 95%, at least 96%, at least 97%, at least 98%, or at least 99% to any one of SEQ ID NOs: 25-27 or 50.

65. The prime editor of any one of claims 38-64, wherein the prime editor comprises the structure NH₂-[bipartite NLS]-[Cas9]-[linker]-[reverse transcriptase]-[bipartite NLS]-[NLS].

66. The prime editor of any one of claims 38-65, wherein the prime editor comprises the fusion protein architecture of PEmax.

67. The prime editor of any one of claims 38-66, wherein the prime editor is smaller in size than PE2, and wherein the prime editor has an editing efficiency comparable to that of PE2.

68. The prime editor of any one of claims 38-67, wherein the prime editor has an increased editing efficiency compared to PEmax for edits that require structured pegRNA reverse transcriptase templates (RTTs).

69. A fusion protein comprising a Cas9 variant of any one of claims 14-37 and an effector domain.

70. The fusion protein of claim 69, wherein the effector domain comprises nuclease activity, nickase activity, recombinase activity, deaminase activity, methyltransferase activity, methylase activity, acetylase activity, acetyltransferase activity, transcriptional activation activity, transcriptional repression activity, or polymerase activity.

71. A reverse transcriptase variant comprising the sequence of any one of SEQ ID NOs: 25-27 or 50, or a sequence at least 80%, at least 85%, at least 90%, at least 95%, at least 96%, at least 97%, at least 98%, or at least 99% to any one of SEQ ID NOs: 25-27 or 50.

72. A Cas9 variant comprising the sequence of any one of SEQ ID NOs: 28, 48, 49, or 145, or a sequence at least 80%, at least 85%, at least 90%, at least 95%, at least 96%, at least 97%, at least 98%, or at least 99% to any one of SEQ ID NOs: 28, 48, 49, or 145.

73. A prime editor comprising the sequence of any one of SEQ ID NOs: 155-158, or a sequence at least 80%, at least 85%, at least 90%, at least 95%, at least 96%, at least 97%, at least 98%, or at least 99% to any one of SEQ ID NOs: 155-158.

74. A complex comprising the prime editor of any one of claims 38-68 or the fusion protein of claim 69 or 70 and a pegRNA, optionally wherein the pegRNA is an epegRNA.

75. A polynucleotide encoding the reverse transcriptase variant of any one of claims 1-13 or 71.

76. A polynucleotide encoding the Cas9 variant of any one of claims 14-37 or 72.

77. One or more polynucleotides encoding the prime editor of any one of claims 38-68 or the fusion protein of claim 69 or 70.

78. A vector comprising the polynucleotide of claim 75 and/or the polynucleotide of claim 76.

79. The vector of claim 78 further comprising a polynucleotide encoding a pegRNA.

80. One or more vectors comprising the one or more polynucleotides of claim 77.

81. The one or more vectors of claim 80 further comprising a polynucleotide encoding a pegRNA.

82. One or more AAV particles comprising the one or more polynucleotides of any one of claims 75-77 or the one or more vectors of any one of claims 78-81.

83. The one or more AAV particles of claim 82, wherein the AAV particles comprise AAV1, AAV2, AAV3, AAV4, AAV5, AAV6, AAV7, AAV8, or AAV9.

84. The one or more AAV particles of claim 82 or 83, wherein the AAV particles comprise AAV9.

85. The one or more AAV particles of any one of claims 82-84, comprising a first AAV particle and a second AAV particle, wherein the first AAV particle comprises a polynucleotide comprising the structure 5′-[inverted terminal repeat (ITR) sequence]-[promoter]-[Cas9 N-terminal fragment]-[N-intein]-[terminator sequence]-[ITR sequence]-3′, and wherein the second AAV particle comprises a polynucleotide comprising the structure 5′-[ITR sequence]-[promoter]-[C-intein]-[Cas9 C-terminal fragment]-[reverse transcriptase]-[terminator sequence]-[optional nicking gRNA]-[pegRNA]-[ITR]-3′.

86. A cell comprising a reverse transcriptase variant of any one of claims 1-13 or 71, a Cas9 variant of any one of claims 14-37 or 72, a prime editor of any one of claims 38-68 or 73, a fusion protein of claim 69 or 70, a complex of claim 74, the one or more polynucleotides of any one of claims 75-77, the one or more vectors of any one of claims 78-81, or the one or more AAV particles of any one of claims 82-85.

87. A pharmaceutical composition comprising a reverse transcriptase variant of any one of claims 1-13 or 71, a Cas9 variant of any one of claims 14-37 or 72, a prime editor of any one of claims 38-68 or 73, a fusion protein of claim 69 or 70, a complex of claim 74, the one or more polynucleotides of any one of claims 75-77, the one or more vectors of any one of claims 78-81, the one or more AAV particles of any one of claims 82-85, or the cell of claim 86.

88. A method for editing a nucleic acid molecule by prime editing comprising contacting a nucleic acid molecule with a prime editor of any one of claims 38-68 or 73, a complex of claim 74, one or more polynucleotides of any one of claims 75-77, or one or more vectors of any one of claims 78-81, thereby installing one or more modifications to the nucleic acid molecule at a target site.

89. A method for simultaneously editing both strands of a double-stranded nucleic acid molecule at a target site to be edited comprising contacting the double-stranded nucleic acid molecule with:
(a) a prime editor of any one of claims 38-68, or a polynucleotide encoding the prime editor;
(b) a first prime editing guide RNA (first pegRNA), or a polynucleotide encoding the first pegRNA that comprises
(i) a first spacer sequence that binds to a first binding site on a first strand of the double-stranded DNA sequence upstream of the target site relative to the second strand,
(ii) a first gRNA core that is capable of complexing with the prime editor, and
(iii) a first DNA synthesis template that encodes a first single-stranded DNA sequence,
and
(c) a second prime editing guide RNA (second pegRNA), or a polynucleotide encoding the second pegRNA, that comprises
(i) a second spacer sequence that binds to a second binding site on a second strand of the double-stranded DNA sequence downstream of the target site relative to the second strand;
(ii) a second gRNA core that is capable of complexing with the prime editor, and
(iii) a second DNA synthesis template that encodes a second single-stranded DNA sequence.

90. The method of claim 88 or 89, wherein the method further comprises contacting the nucleic acid molecule with one or more second strand nicking gRNAs.

91. The method of any one of claims 88-90, wherein the method comprises installing a recombinase recognition site in the nucleic acid molecule.

92. A kit comprising a reverse transcriptase variant of any one of claims 1-13 or 71, a Cas9 variant of any one of claims 14-37 or 72, a prime editor of any one of claims 38-68 or 73, a fusion protein of claim 69 or 70, a complex of claim 74, the one or more polynucleotides of any one of claims 75-77, the one or more vectors of any one of claims 78-81, the one or more AAV particles of any one of claims 82-85, or the cell of claim 86.

93. Use of a reverse transcriptase variant of any one of claims 1-13 or 71, a Cas9 variant of any one of claims 14-37 or 72, a prime editor of any one of claims 38-68 or 73, a fusion protein of claim 69 or 70, a complex of claim 74, the one or more polynucleotides of any one of claims 75-77, the one or more vectors of any one of claims 78-81, the one or more AAV particles of any one of claims 82-85, or the cell of claim 86 in the manufacture of a medicament.

94. The reverse transcriptase variant of any one of claims 1-13 or 71, the Cas9 variant of any one of claims 14-37 or 72, a prime editor of any one of claims 38-68 or 73, a fusion protein of claim 69 or 70, the complex of claim 74, the one or more polynucleotides of any one of claims 75-77, the one or more vectors of any one of claims 78-81, the one or more AAV particles of any one of claims 82-85, or the cell of claim 86 for use in medicine.

95. A system of polynucleotides for phage-assisted continuous and non-continuous evolution of prime editors comprising:
i) a first polynucleotide encoding a pegRNA and the gIII gene;
ii) a second polynucleotide encoding a Cas9 protein fused to an N-intein;
iii) a third polynucleotide encoding an RNA polymerase;
iv) a fourth polynucleotide encoding proteins capable of mutagenizing a phage, optionally wherein the fourth polynucleotide comprises the MP6 plasmid; and
v) a fifth polynucleotide encoding a reverse transcriptase fused to a C-intein.

96. A system of polynucleotides for phage-assisted continuous and non-continuous evolution of prime editors comprising:
i) a first polynucleotide encoding a pegRNA and the gIII gene;
ii) a second polynucleotide encoding a prime editor;
iii) a third polynucleotide encoding an RNA polymerase; and
iv) a fourth polynucleotide encoding proteins capable of mutagenizing a phage, optionally wherein the fourth polynucleotide comprises the MP6 plasmid.


SEQUENCE LISTING (158 sequences)

SEQ ID NO: 1 - protein, 505 aa, unidentified
  source 1..505: note = Schizosaccharomyces pombe
ISSSKHTLSQMNKVSNIVKEPELPDIYKEFKDITADTNTEKLPKPIKGLEFEVELTQENYRLPIRNYPLPPGKMQAMNDEINQGLKSGIIRESKAINACPVMFVPKKEGTLRMVVDYKPLNKYVKPNIYPLPLIEQLLAKIQGSTIFTKLDLKSAYHLIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGFTPCQENIDKVLQWKQPKNRKELRQFLGSVNYLRKFIPKTSQLTHPLNKLLKKDVRWKWTPTQTQAIENIKQCLVSPPVLRHFDFSKKILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 2 - protein, 1368 aa, Synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 2117 aa, Synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 4 - protein, 2117 aa, Synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 5 - protein, 2131 aa, Synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLD

SEQ ID NO: 6 - protein, 1368 aa, Synthetic construct [sequence omitted]

SEQ ID NO: 7 - protein, 407 aa, Synthetic construct
GRPYVTLNLNGMFMDKFKPYSKSNAPITTLEKLSKALSISVEELKAIAELSLDEKYTLKEIPKIDGSKRIVYSLHPKMRLLQSRINKRIFKELVVFPSFLFGSVPSKNDVLNSNVKRDYVSCAKAHCGAKTVLKVDISNFFDNIHRDLVRSVFEEILHIKDEALEYLVDICTKDDFVVQGALTSSYIATLCLFAVEGDVVRRAQRKGLVYTRLVDDITVSSKISNYDFSQMQSHIERMLSEHDLPINKHKTKIFHCSSEPIKVHGLRVDYDSPRLPSDEVKRIRASIHNLKLLAAKNNTKTSVAYRKEFNRCMGRVNKLGRVGHEKYESFKKQLQAIKPMPSKRDVAVIDAAIKSLELSYSKGNQNKHWYKRKYDLTRYKMIILTRSESFKEKLECFKSRLASLKPL

SEQ ID NO: 8 - protein, 1367 aa, Synthetic construct
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 9 - protein, 1404 aa, Synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSSDYKDHDGDYKDHDIDYKDDDDKAAG

SEQ ID NO: 10 - protein, 1367 aa, Synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 11 - protein, 1367 aa, Synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 12 - protein, 1378 aa, Synthetic construct
MKEYHIGLDIGTSSIGWAVTDSQFKLMRIKGKTAIGVRLFEEGKTAAERRTFRTTRRRLKRRKWRLHYLDEIFAPHLQEVDENFLRRLKQSNIHPEDPTKNQAFIGKLLFPDLLKKNERGYPTLIKMRDELPVEQRAHYPVMNIYKLREAMINEDRQFDLREVYLAVHHIVKYRGHFLNNASVDKFKVGRIDFDKSFNVLNEAYEELQNGEGSFTIEPSKVEKIGQLLLDTKMRKLDRQKAVAKLLEVKVADKEETKRNKQIATAMSKLVLGYKADFATVAMANGNEWKIDLSSETSEDEIEKFREELSDAQNDILTEITSLFSQIMLNEIVPNGMSISESMMDRYWTHERQLAEVKEYLATQPASARKEFDQVYNKYIGQAPKERGFDLEKGLKKILSKKENWKEIDELLKAGDFLPKQRTSANGVIPHQMHQQELDRIIEKQAKYYPWLATENPATGERDRHQAKYELDQLVSFRIPYYVGPLVTPEVQKATSGAKFAWAKRKEDGEITPWNLWDKIDRAESAEAFIKRMTVKDTYLLNEDVLPANSLLYQKYNVLNELNNVRVNGRRLSVGIKQDIYTELFKKKKTVKASDVASLVMAKTRGVNKPSVEGLSDPKKFNSNLATYLDLKSIVGDKVDDNRYQTDLENIIEWRSVFEDGEIFADKLTEVEWLTDEQRSALVKKRYKGWGRLSKKLLTGIVDENGQRIIDLMWNTDQNFKEIVDQPVFKEQIDQLNQKAITNDGMTLRERVESVLDDAYTSPQNKKAIWQVVRVVEDIVKAVGNAPKSISIEFARNEGNKGEITRSRRTQLQKLFEDQAHELVKDTSLTEELEKAPDLSDRYYFYFTQGGKDMYTGDPINFDEISTKYDIDHILPQSFVKDNSLDNRVLTSRKENNKKSDQVPAKLYAAKMKPYWNQLLKQGLITQRKFENLTKDVDQNIKYRSLGFVKRQLVETRQVIKLTANILGSMYQEAGTEIIETRAGLTKQLREEFDLPKVREVNDYHHAVDAYLTTFAGQYLNRRYPKLRSFFVYGEYMKFKHGSDLKLRNFNFFHELMEGDKSQGKVVDQQTGELITTRDEVAKSFDRLLNMKYMLVSKEVHDRSDQLYGATIVTAKESGKLTSPIEIKKNRLVDLYGAYTNGTSAFMTIIKFTGNKPKYKVIGIPTTSAASLKRAGKPGSESYNQELHRIIKSNPKVKKGFEIVVPHVSYGQLIVDGDCKFTLASPTVQHPATQLVLSKKSLETISSGYKILKDKPAIANERLIRVFDEVVGQMNRYFTIFDQRSNRQKVADARDKFLSLPTESKYEGAKKVQVGKTEVITNLLMGLHANATQGDLKVLGLATFGFFQSTTGLSLSEDTMIVYQSPTGLFERRICLKDI

SEQ ID NO: 13 - protein, 1053 aa, Synthetic construct
MGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKK

SEQ ID NO: 14 - protein, 1409 aa, Synthetic construct
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 15 - protein, 1402 aa, Synthetic construct
MKIKNYNLALTPSTSAVGHVEVDDDLNILEPVHHQKAIGVAKFGEGETAEARRLARSARRTTKRRANRINHYFNEIMKPEIDKVDPLMFDRIKQAGLSPLDERKEFRTVIFDRPNIASYYHNQFPTIWHLQKYLMITDEKADIRLIYWALHSLLKHRGHFFNTTPMSQFKPGKLNLKDDMLALDDYNDLEGLSFAVANSPEIEKVIKDRSMHKKEKIAELKKLIVNDVPDKDLAKRNNKIITQIVNAIMGNSFHLNFIFDMDLDKLTSKAWSFKLDDPELDTKFDAISGSMTDNQIGIFETLQKIYSAISLLDILNGSSNVVDAKNALYDKHKRDLNLYFKFLNTLPDEIAKTLKAGYTLYIGNRKKDLLAARKLLKVNVAKNFSQDDFYKLINKELKSIDKQGLQTRFSEKVGELVAQNNFLPVQRSSDNVFIPYQLNAITFNKILENQGKYYDFLVKPNPAKKDRKNAPYELSQLMQFTIPYYVGPLVTPEEQVKSGIPKTSRFAWMVRKDNGAITPWNFYDKVDIEATADKFIKRSIAKDSYLLSELVLPKHSLLYEKYEVFNELSNVSLDGKKLSGGVKQILFNEVFKKTNKVNTSRILKALAKHNIPGSKITGLSNPEEFTSSLQTYNAWKKYFPNQIDNFAYQQDLEKMIEWSTVFEDHKILAKKLDEIEWLDDDQKKFVANTRLRGWGRLSKRLLTGLKDNYGKSIMQRLETTKANFQQIVYKPEFREQIDKISQAAAKNQSLEDILANSYTSPSNRKAIRKTMSVVDEYIKLNHGKEPDKIFLMFQRSEQEKGKQTEARSKQLNRILSQLKADKSANKLFSKQLADEFSNAIKKSKYKLNDKQYFYFQQLGRDALTGEVIDYDELYKYTVLHIIPRSKLTDDSQNNKVLTKYKIVDGSVALKFGNSYSDALGMPIKAFWTELNRLKLIPKGKLLNLTTDFSTLNKYQRDGYIARQLVETQQIVKLLATIMQSRFKHTKIIEVRNSQVANIRYQFDYFRIKNLNEYYRGFDAYLAAVVGTYLYKVYPKARRLFVYGQYLKPKKTNQENQDMHLDSEKKSQGFNFLWNLLYGKQDQIFVNGTDVIAFNRKDLITKMNTVYNYKSQKISLAIDYHNGAMFKATLFPRNDRDTAKTRKLIPKKKDYDTDIYGGYTSNVDGYMLLAEIIKRDGNKQYGFYGVPSRLVSELDTLKKTRYTEYEEKLKEIIKPELGVDLKKIKKIKILKNKVPFNQVIIDKGSKFFITSTSYRWNYRQLILSAESQQTLMDLVVDPDFSNHKARKDARKNADERLIKVYEEILYQVKNYMPMFVELHRCYEKLVDAQKTFKSLKISDKAMVLNQILILLHSNATSPVLEKLGYHTRFTLGKKHNLISENAVLVTQSITGLKENHVSIKQML

SEQ ID NO: 16 - protein, 1385 aa, Synthetic construct
MTNEKYSIGLDIGTSSIGFAVVNDNNRVIRVKGKNAIGVRLFDEGKAAADRRSFRTTRRSFRTTRRRLSRRRWRLKLLREIFDAYITPVDEAFFIRLKESNLSPKDSKKQYSGDILFNDRSDKDFYEKYPTIYHLRNALMTEHRKFDVREIYLAIHHIMKFRGHFLNATPANNFKVGRLNLEEKFEELNDIYQRVFPDESIEFRTDNLEQIKEVLLDNKRSRADRQRTLVSDIYQSSEDKDIEKRNKAVATEILKASLGNKAKLNVITNVEVDKEAAKEWSITFDSESIDDDLAKIEGQMTDDGHEIIEVLRSLYSGITLSAIVPENHTLSQSMVAKYDLHKDHLKLFKKLINGMTDTKKAKNLRAAYDGYIDGVKGKVLPQEDFYKQVQVNLDDSAEANEIQTYIDQDIFMPKQRTKANGSIPHQLQQQELDQIIENQKAYYPWLAELNPNPDKKRQQLAKYKLDELVTFRVPYYVGPMITAKDQKNQSGAEFAWMIRKEPGNITPWNFDQKVDRMATANQFIKRMTTTDTYLLGEDVLPAQSLLYQKFEVLNELNKIRIDHKPISIEQKQQIFNDLFKQFKNVTIKHLQDYLVSQGQYSKRPLIEGLADEKRFNSSLSTYSDLCGIFGAKLVEENDRQEDLEKIIEWSTIFEDKKIYRAKLNDLTWLTDDQKEKLATKRYQGWGRLSRKLLVGLKNSEHRNIMDILWITNENFMQIQAEPDFAKLVTDANKGMLEKTDSQDVINDLYTSPQNKKAIRQILLVVHDIQNAMHGQAPAKIHVEFARGEERNPRRSVQRQRQVEAAYEKVSNELVSAKVRQEFKEAINNKRDFKDRLFLYFMQGGIDIYTGKQLNIDQLSSYQIDHILPQAFVKDDSLTNRVLTNENQVKADSVPIDIFGKKMLSVWGRMKDQGLISKGKYRNLTMNPENISAHTENGFINRQLVETRQVIKLAVNILADEYGDSTQIISVKADLSHQMREDFELLKNRDVNDYHHAFDAYLAAFIGNYLLKRYPKLESYFVYGDFKKFTQKETKMRRFNFIYDLKHCDQVVNKETGEILWTKDEDIKYIRHLFAYKKILVSHEVREKRGALYNQTIYKAKDDKGSGQESKKLIRIKDDKETKIYGGYSGKSLAYMTIVQITKKNKVSYRVIGIPTLALARLNKLENDSTENNGELYKIIKPQFTHYKVDKKNGEIIETTDDFKIVVSKVRFQQLIDDAGQFFMLASDTYKNNAQQLVISNNALKAINNTNITDCPRDDLERLDNLRLDSAFDEIVKKMDKYFSAYDANNFREKIRNSNLIFYQLPVEDQWENNKITELGKRTVLTRILQGLHANATTTDMSIFKIKTPFGQLRQRSGISLSENAQLIYQSPTGLFERRVQLNKIK

SEQ ID NO: 17 - protein, 1367 aa, Synthetic construct
MKKQKFSDYYLGFDIGTNSVGWCVTDLDYNVLRFNKKDMWGSRLFEEAKTAAERRVQRNSRRRLKRRKWRLNLLEEIFSNEILKIDSNFFRRLKESSLWLEDKSSKEKFTLFNDDNYKDYDFYKQYPTIFHLRNELIKNPEKKDIRLVYLAIHSIFKSRGHFLFEGQNLKEIKNFETLYNNLIAFLEDNGINKIIDKNNIEKLEKIVCDSKKGLKDKEKEFKEIFNSDKQLVAIFKLSVGSSVSLNDLFDTDEYKKGEVEKEKISFREQIYEDDKPIYYSILGEKIELLDIAKTFYDFMVLNNILADSQYISEAKVKLYEEHKKDLKNLKYIIRKYNKGNYDKLFKDKNENNYSAYIGLNKEKSKKEVIEKSRLKIDDLIKNIKGYLPKVEEIEEKDKAIFNKILNKIELKTILPKQRISDNGTLPYQIHEAELEKILENQSKYYDFLNYEENGIITKDKLLMTFKFRIPYYVGPLNSYHKDKGGNSWIVRKEEGKILPWNFEQKVDIEKSAEEFIKRMTNKCTYLNGEDVIPKDTFLYSEYVILNELNKVQVNDEFLNEENKRKIIDELFKENKKVSEKKFKEYLLVKQIVDGTIELKGVKDSFNSNYISYIRFKDIFGEKLNLDIYKEISEKSILWKCLYGDDKKIFEKKIKNEYGDILTKDEIKKINTFKFNNWGRLSEKLLTGIEFINLETGECYSSVMDALRRTNYNLMELLSSKFTLQESINNENKEMNEASYRDLIEESYVSPSLKRAIFQTLKIYEEIRKITGRVPKKVFIEMARGGDESMKNKKIPARQEQLKKLYDSCGNDIANFSIDIKEMKNSLISYDNNSLRQKKLYLYYLQFGKCMYTGREIDLDRLLQNNDTYDIDHIYPRSKVIKDDSFDNLVLVLKNENAEKSNEYPVKKEIQEKMKSFWRFLKEKNFISDEKYKRLTGKDDFELRGFMARQLVNVRQTTKEVGKILQQIEPEIKIVYSKAEIASSFREMFDFIKVRELNDTHHAKDAYLNIVAGNVYNTKFTEKPYRYLQEIKENYDVKKIYNYDIKNAWDKENSLEIVKKNMEKNTVNITRFIKEKKGQLFDLNPIKKGETSNEIISIKPKVYNGKDDKLNEKYGYYKSLNPAYFLYVEHKEKNKRIKSFERVNLVDVNNIKDEKSLVKYLIENKKLVEPRVIKKVYKRQVILINDYPYSIVTLDSNKLMDFENLKPLFLENKYEKILKNVIKFLEDNQGKSEENYKFIYLKKKDRYEKNETLESVKDRYNLEFNEMYDKFLEKLDSKDYKNYMNNKKYQELLDVKEKFIKLNLFDKAFTLKSFLDLFNRKTMADFSKVGLTKYLGKIQKISSNVLSKNELYLLEESVTGLFVKKIKL

SEQ ID NO: 18 - protein, 1286 aa, Synthetic construct
RRKQRIQILQELLGEEVLKTDPGFFHRMKESRYVVEDKRTLDGKQVELPYALFVDKDYTDKEYYKQFPTINHLIVYLMTTSDTPDIRLVYLALHYYMKNRGNFLHSGDINNVKDINDILEQLDNVLETFLDGWNLKLKSYVEDIKNIYNRDLGRGERKKAFVNTLGAKTKAEKAFCSLISGGSTNLAELFDDSSLKEIETPKIEFASSSLEDKIDGIQEALEDRFAVIEAAKRLYDWKTLTDILGDSSSLAEARVNSYQMHHEQLLELKSLVKEYLDRKVFQEVFVSLNVANNYPAYIGHTKINGKKKELEVKRTKRNDFYSYVKKQVIEPIKKKVSDEAVLTKLSEIESLIEVDKYLPLQVNSDNGVIPYQVKLNELTRIFDNLENRIPVLRENRDKIIKTFKFRIPYYVGSLNGVVKNGKCTNWMVRKEEGKIYPWNFEDKVDLEASAEQFIRRMTNKCTYLVNEDVLPKYSLLYSKYLVLSELNNLRIDGRPLDVKIKQDIYENVFKKNRKVTLKKIKKYLLKEGIITDDDELSGLADDVKSSLTAYRDFKEKLGHLDLSEAQMENIILNITLFGDDKKLLKKRLAALYPFIDDKSLNRIATLNYRDWGRLSERFLSGITSVDQETGELRTIIQCMYETQANLMQLLAEPYHFVEAIEKENPKVDLESISYRIVNDLYVSPAVKRQIWQTLLVIKDIKQVMKHDPERIFIEMAREKQESKKTKSRKQVLSEVYKKAKEYEHLFEKLNSLTEEQLRSKKIYLYFTQLGKCMYSGEPIDFENLVSANSNYDIDHIYPQSKTIDDSFNNIVLVKKSLNAYKSNHYPIDKNIRDNEKVKTLWNTLVSKGLITKEKYERLIRSTPFSDEELAGFIARQLVETRQSTKAVAEILSNWFPESEIVYSKAKNVSNFRQDFEILKVRELNDCHHAHDAYLNIVVGNAYHTKFTNSPYRFIKNKANQEYNLRKLLQKVNKIESNGVVAWVGQSENNPGTIATVKKVIRRNTVLISRMVKEVDGQLFDLTLMKKGKGQVPIKSSDERLTDISKYGGYNKATGAYFTFVKSKKRGKVVRSFEYVPLHLSKQFENNNELLKEYIEKDRGLTDVEILIPKVLINSLFRYNGSLVRITGRGDTRLLLVHEQPLYVSNSFVQQLKSVSSYKLKKSENDNAKLTKTATEKLSNIDELYDGLLRKLDLPIYSYWFSSIKEYLVESRTKYIKLSIEEKALVIFEILHLFQSDAQVPNLKILGLSTKPSRIRIQKNLKDTDKMSIIHQSPSGIFEHEIELTSL

SEQ ID NO: 19 - protein, 1312 aa, Synthetic construct
MQNGFLGITVSSEQVGWAVTNPKYELERASRKDLWGVRLFDKAETAEDRRMFRTNRRLNQRKKNRIHYLRDIFHEEVNQKDPNFFQQLDESNFCEDDRTVEFNFDTNLYKNQFPTVYHLRKYLMETKDKPDIRLVYLAFSKFMKNRGHFLYKGNLGEVMDFENSMKGFCESLEKFNIDFPTLSDEQVKEVRDILCDHKIAKTVKKKNIITITKVKSKTAKAWIGLFCGCSVPVKVLFQDIDEEIVTDPEKISFEDASYDDYIANIEKGVGIYYEAIVSAKMLFDWSILNEILGDHQLLSDAMIAEYNKHHDDLKRLQKIIKGTGSRELYQDIFINDVSGNYVCYVGHAKTMSSADQKQFYTFLKNRLKNVNGISSEDAEWIDTEIKNGTLLPKQTKRDNSVIPHQLQLREFELILDNMQEMYPFLKENREKLLKIFNFVIPYYVGPLKGVVRKGESTNWMVPKKDGVIHPWNFDEMVDKEASAECFISRMTGNCSYLFNEKVLPKNSLLYETFEVLNELNPLKINGEPISVELKQRIYEQLFLTGKKVTKKSLTKYLIKNGYDKDIELSGIDNEFHSNLKSHIDFEDYDNLSDEEVEQIILRITVFEDKQLLKDYLNREFVKLSEDERKQICSLSYKGWGNLSEMLLNGITVTDSNGVEVSVMDMLWNTNLNLMQILSKKYGYKAEIEHYNKEHEKTIYNREDLMDYLNIPPAQRRKVNQLITIVKSLKKTYGVPNKIFFKISREHQDDPKRTSSRKEQLKYLYKSLKSEDEKHLMKELDELNDHELSNDKVYLYFLQKGRCIYSGKKLNLSRLRKSNYQNDIDYIYPLSAVNDRSMNNKVLTGIQENRADKYTYFPVDSEIQKKMKGFWMELVLQGFMTKEKYFRLSRENDFSKSELVSFIEREISDNQQSGRMIASVLQYYFPESKIVFVKEKLISSFKRDFHLISSYGHNHLQAAKDAYITIVVGNVYHTKFTMDPAIYFKNHKRKDYDLNRLFLENISRDGQIAWESGPYGSIQTVRKEYAQNHIAVTKRVVEVKGGLFKQMPLKKGHGEYPLKTNDPRFGNIAQYGGYTNVTGSYFVLVESMEKGKKRISLEYVPVYLHERLEDDPGHKLLKEYLVDHRKLNHPKILLAKVRKNSLLKIDGFYYRLNGRSGNALILTNAVELIMDDWQTKTANKISGYMKRRAIDKKARVYQNEFHIQELEQLYDFYLDKLKNGVYKNRKNNQAELIHNEKEQFMELKTEDQCVLLTEIKKLFVCSPMQADLTLIGGSKHTGMIAMSSNVTKADFAVIAEDPLGLRNKVIYSHKGEK

SEQ ID NO: 20 - protein, 1344 aa, Synthetic construct
MSQNNNKIYNIGLDIGDASVGWAVVDEHYNLLKRHGKHMWGSRLFTQANTAVERRSSRSTRRRYNKRRERIRLLREIMEDMVLDVDPTFFIRLANVSFLDQEDKKDYLKENYHSNYNLFIDKDFNDKTYYDKYPTIYHLRKHLCESKEKEDPRLIYLALHHIVKYRGNFLYEGQKFSMDVSNIEDKMIDVLRQFNEINLFEYVEDRKKIDEVLNVLKEPLSKKHKAEKAFALFDTTKDNKAAYKELCAALAGNKFNVTKMLKEAELHDEDEKDISFKFSDATFDDAFVEKQPLLGDCVEFIDLLHDIYSWVELQNILGSAHTSEPSISAAMIQRYEDHKNDLKLLKDVIRKYLPKKYFEVFRDEKSKKNNYCNYINHPSKTPVDEFYKYIKKLIEKIDDPDVKTILNKIELESFMLKQNSRTNGAVPYQMQLDELNKILENQSVYYSDLKDNEDKIRSILTFRIPYYFGPLNITKDRQFDWIIKKEGKENERILPWNANEIVDVDKTADEFIKRMRNFCTYFPDEPVMAKNSLTVSKYEVLNEINKLRINDHLIKRDMKDKMLHTLFMDHKSISANAMKKWLVKNQYFSNTDDIKIEGFQKENACSTSLTPWIDFTKIFGKINESNYDFIEKIIYDVTVFEDKKILRRRLKKEYDLDEEKIKKILKLKYSGWSRLSKKLLSGIKTKYKDSTRTPETVLEVMERTNMNLMQVINDEKLGFKKTIDDANSTSVSGKFSYAEVQELAGSPAIKRGIWQALLIVDEIKKIMKHEPAHVYIEFARNEDEKERKDSFVNQMLKLYKDYDFEDETEKEANKHLKGEDAKSKIRSERLKLYYTQMGKCMYTGKSLDIDRLDTYQVDHIVPQSLLKDDSIDNKVLVLSSENQRKLDDLVIPSSIRNKMYGFWEKLFNNKIISPKKFYSLIKTEFNEKDQERFINRQIVETRQITKHVAQIIDNHYENTKVVTVRADLSHQFRERYHIYKNRDINDFHHAHDAYIATILGTYIGHRFESLDAKYIYGEYKRIFRNQKNKGKEMKKNNDGFILNSMRNIYADKDTGEIVWDPNYIDRIKKCFYYKDCFVTKKLEENNGTFFNVTVLPNDTNSDKDNTLATVPVNKYRSNVNKYGGFSGVNSFIVAIKGKKKKGKKVIEVNKLTGIPLMYKNADEEIKINYLKQAEDLEEVQIGKEILKNQLIEKDGGLYYIVAPTEIINAKQLILNESQTKLVCEIYKAMKYKNYDNLDSEKIIDLYRLLINKMELYYPEYRKQLVKKFEDRYEQLKVISIEEKCNIIKQILATLHCNSSIGKIMYSDFKISTTIGRLNGRTISLDDISFIAESPTGMYSKKYKL

SEQ ID NO: 21 - protein, 1288 aa, Synthetic construct
MRLFEEGHTAEDRRLKRTARRRISRRRNRLRYLQAFFEEAMTDLDENFFARLQESFLVPEDKKWHRHPIFAKLEDEVAYHETYPTIYHLRKKLADSSEQADLRLIYLALAHIVKYRGHFLIEGKLSTENTSVKDQFQQFMVIYNQTFVNGESRLVSAPLPESVLIEEELTEKASRTKKSEKVLQQFPQEKANGLFGQFLKLMVGNKADFKKVFGLEEEAKITYASESYEEDLEGILAKVGDEYSDVFLAAKNVYDAVELSTILADSDKKSHAKLSSSMIVRFTEHQEDLKKFKRFIRENCPDEYDNLFKNEQKDGYAGYIAHAGKVSQLKFYQYVKKIIQDIAGAEYFLEKIAQENFLRKQRTFDNGVIPHQIHLAELQAIIHRQAAYYPFLKENQEKIEQLVTFRIPYYVGPLSKGDASTFAWLKRQSEEPIRPWNLQETVDLDQSATAFIERMTNFDTYLPSEKVLPKHSLLYEKFMVFNELTKISYTDDRGIKANFSGKEKEKIFDYLFKTRRKVKKKDIIQFYRNEYNTEIVTLSGLEEDQFNASFSTYQDLLKCGLTRAELDHPDNAEKLEDIIKILTIFEDRQRIRTQLSTFKGQFSAEVLKKLERKHYTGWGRLSKKLINGIYDKESGKTILDYLVKDDGVSKHYNRNFMQLINDSQLSFKNAIQKAQSSEHEETLSETVNELAGSPAIKKGIYQSLKIVDELVAIMGYAPKRIVVEMARENQTTSTGKRRSIQRLKIVEKAMAEIGSNLLKEQPTTNEQLRDTRLFLYYMQNGKDMYTGDELSLHRLSHYDIDHIIPQSFMKDDSLDNLVLVGSTENRGKSDDVPSKEVVKDMKAYWEKLYAAGLISQRKFQRLTKGEQGGLTLEDKAHFIQRQLVETRQITKNVAGILDQRYNAKSKEKKVQIITLKASLTSQFRSIFGLYKVREVNDYHHGQDAYLNCVVATTLLKVYPNLAPEFVYGEYPKFQTFKENKATAKAIIYTNLLRFFTEDEPRFTKDGEILWSNSYLKTIKKELNYHQMNIVKKVEVQKGGFSKESIKPKGPSNKLIPVKNGLDPQKYGGFDSPVVAYTVLFTHEKGKKPLIKQEILGITIMEKTRFEQNPILFLEEKGFLRPRVLMKLPKYTLYEFPEGRRRLLASAKEAQKGNQMVLPEHLLTLLYHAKQCLLPNQSESLAYVEQHQPEFQEILERVVDFAEVHTLAKSKVQQIVKLFEANQTADVKEIAASFIQLMQFNAMGAPSTFKFFQKDIERARYTSIKEIFDATIIYQSPTGLYETRRKVVD

SEQ ID NO: 22 - protein, 1052 aa, Synthetic construct
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 23 - protein, 1082 aa, Synthetic construct
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 24 - protein, 1375 aa, Streptococcus canis
MEKKYSIGLDIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDHIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGD

SEQ ID NO: 25 - protein, 505 aa, Synthetic construct
ISSSKHTLSQMNKVSNIVKEPELPDIYKEFKDITADTNTEKLPKPIKGLEFEVELTQENYRLPIRNYPLTPVKMQAMNDEINQGLKGGIIRESKAINACPVIFVPRKEGTLRMVVDYRPLNKYVKPNVYPLPLIEQLLAKIQGSTIFTKLDLKSAYHQIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGLTPCQENIDKVLQWKQPKNRKELRQFLGSVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISI

SEQ ID NO: 26 - protein, 505 aa, Synthetic construct
ISSSKHTLSQMNKVSNIVKEPELPDIYKEFKDITADTNTEKLPKPIKGLEFEVELTQENYRLPIRNYPLTPVKMQAMNDEINQGLKGGIIRESKAINACPVIFVPRKEGTLRMVVDYRPLNKYVKPNVYPLPLIEQLLAKIQGSTIFTKLDLKSAYHQIRVRKGDEHKLAFRCPRGVFEYLVMPYGIKTAPAHFQYFINTILGEAKESHVVCYMDDILIHSKSESEHVKHVKDVLQKLKNANLIINQAKCEFHQSQVKFLGYHISEKGLTPCQENIDKVLQWKQPKNQKELRQFLGQVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISI

SEQ ID NO: 27 - protein, 497 aa, Synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPNVPNPYNLLSGLPPSHQWYTVLDLKDAFFCLRLHPTSQPLFAFEWRDPEMGISGQLTWTRLPQGFKNSPTLFCEALHRDLADFRIQHPDLILLQYYDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLD

SEQ ID NO: 28 - protein, 1368 aa, Synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQRNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIARQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 29 - protein, 677 aa, Synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 30 - protein, 677 aa, Synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 31 - DNA, 40 bp, Synthetic construct
  source 1..40: mol_type = other DNA
ctctcgttgtccaggtatggcccggctccactgccctccc

SEQ ID NO: 32 - protein, 677 aa, Synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 33 - protein, 677 aa, Synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 34 - protein, 677 aa, Synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 35 - protein, 677 aa, Synthetic construct
TLNIEDEYRLHETSKEPDVSLGSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 36 - protein, 677 aa, Synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 37 - protein, 677 aa, Synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSP

SEQ ID NO: 38 - protein, 677 aa, Synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 39 - protein, 677 aa, Synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 40 - protein, 677 aa, Synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSKARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 41 - protein, 677 aa, Synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 42 - protein, 677 aa, Synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 43 - protein, 677 aa, Synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 44 - protein, 677 aa, Synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGTAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 45 - protein, 677 aa, Synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSP

SEQ ID NO: 46 - protein, 677 aa, Synthetic construct
TLNIEDEYRLHETSKEPDVSLGSTWLSDFPQAWAETGGMGLAVRQAPLIILLKATSTPVSIKQYPMKQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPALFDEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGTAGNCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAELIALTQALKMAEGKKLNVYTNSRYAFATAHIQGEIYRRRGLLTSEGKEIKNKDEILALLKALFLPKRLSIIHCPGHQKGHSAEARGNRMANQAARKAAITETPDTSTLLIENSSP

SEQ ID NO: 47 - protein, 677 aa, Synthetic construct
TLNIEDEYRLHETSKEPDVSLGSTWLSDFPQAWAETGGMGLAVRQAPLIILLKATSTPVSIKQYPMKQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPALFNEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGTAGNCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAELIALTQALKMAEGKKLNVYTNSRYAFATAHIQGEIYRRRGLLTSEGKEIKNKDEILALLKALFLPKRLSIIHCPGHQKGHSAEARGNRMANQAARKAAITETPDTSTLLIENSSP

SEQ ID NO: 48 - protein, 1368 aa, Synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFRRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEKTITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMVEERLKTYAHLFDNKVMKQLKRRRYTGWGRLSRKLINGIRDKQSGKTILDFLKSDGFANRNFMQLIHDDSLTFKEDIQKAQVSGQGDSLYEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIARQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 49 - protein, 1368 aa, Synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFRRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEKTITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMVEERLKTYAHLFDNKVMKQLKRCRYTGWGRLSRKLINGIRDKQSGKTILDFLKSDGFANRNFMQLIHDDSLTFKEDIQKAQVSGQGDSLYEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 50 - protein, 407 aa, Synthetic construct
GRPYVTLNLNGMFMDKFKPYSKSNAPITTLEKLSKALSISVEELKAIAELSLDEKYTLKKIPKIDGSKRIVYSLHPKMRLLQSRINERIFKELVVFPSFLFGSVPSKNDVLNSNVKRDYVSCAKAHCGAKTVLKVDISNFFDNIHRDLVRSVFEEILHIKDEALDYLVDICTKDDFVVQGALTSSYIATLCLFAVEGDVVRRAQRKGLVYTRLVDDITVSSKISNYDFSQMQSHIERMLSEHNLPINKHKTKIFHCSSEPIKVHGLIVDYDSPRLPSDKVKRIRASIHNLKLLAAKNNTKTSVAYRKEFNRCMGRVNELGRVGHEKYESFKKQLQAIKPMPSNRDVAVIDAAIKSLELSYSKGNQNKHWYKRKYDLTRYKMIILTRSESFKEKLECFKSRLASLKPL

SEQ ID NO: 51 - protein, 604 aa, Mus musculus
VFTLWGRDIMKDIKVRLMTDSPDDSQDLMIGAIESNLFADQISWKSDQPVWLNQWPLKQEKLQALQQLVTEQLQLGHLEESNSPWNTPVFVIKKKSGKWRLLQDLRAVNATMHDMGALQPGLPSPVAVPKGWEIIIIDLQDCFFNIKLHPEDCKRFAFSVPSPNFKRPYQRFQWKVLPQGMKNSPTLCQKFVDKAILTVRDKYQDSYIVHYMDDILLAHPSRSIVDEILTSMIQALNKHGLVVSTEKIQKYDNLKYLGTHIQGDSVSYQKLQIRTDKLRTLNDFQKLLGNINWIRPFLKLTTGELKPLFEILNGDSNPISTRKLTPEACKALQLMNERLSTARVKRLDLSQPWSLCILKTEYTPTACLWQDGVVEWIHLPHISPKVITPYDIFCTQLIIKGRHRSKELFSKDPDYIVVPYTKVQFDLLLQEKEDWPISLLGFLGEVHFHLPKDPLLTFTLQTAIIFPHMTSTTPLEKGIVIFTDGSANGRSVTYIQGREPIIKENTQNTAQQAEIVAVITAFEEVSQPFNLYTDSKYVTGLFPEIETATLSPRTKIYTELKHLQRLIHKRQEKFYIGHIRGHTGLPGPLAQGNAYADSLTRILT

SEQ ID NO: 52 - protein, 572 aa, unidentified
  source 1..572: note = AVIAN SARCOMA LEUKOSIS VIRUS
TVALHLAIPLKWKPDHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLVVGQVLEPLRLKHPSLRMLHYMDDLLLAASSHDGLEAAGEEVISTLERAGFTISPDKIQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALKGFAGKIRSSDTPSIFDIARPLHVSLKVRVTDHPVPGPTVFTDASSSTHKGVVVWREGPRWEIKEIADSGASVQQLEARAVAMALLLWPTTPTNVVTDSAFVAKMLLKMGQEGVPSTAAAFILEDALSQRSAMAAVLHVRSHSEVPGFFTEGNDVADSQATFQAY

SEQ ID NO: 53 - protein, 667 aa, unidentified
  source 1..667: note = PORCINE ENDOGENOUS RETROVIRUS
TLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPERNWYTVLDLKDAFFCLRLHPTSQPLFAFEWRDPGTGRTGQLTWTRLPQGFKNSPTIFDEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGTAGFCRLWIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTHTIWASSLPEGTSAQKAELMALTQALRLAEGKSINIYTDSRYAFATAHVHGAIYKQRGLLTSAGREIKNKEEILSLLEALHLPKRLAIIHCPGHQKAKDLISRGNQMADRVAKQAAQAVNLLPI

SEQ ID NO: 54 - protein, 625 aa, unidentified
  source 1..625: note = HIV-MMLV
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFKKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIEN

SEQ ID NO: 55 - protein, 693 aa, unidentified
  source 1..693: note = AVIRE
APLEEEYRLFLEAPIQNVTLLEQWKREIPKVWAEINPPGLASTQAPIHVQLLSTALPVRVRQYPITLEAKRSLRETIRKFRAAGILRPVHSPWNTPLLPVRKSGTSEYRMVQDLREVNKRVETIHPTVPNPYTLLSLLPPDRIWYSVLDLKDAFFCIPLAPESQLIFAFEWADAEEGESGQLTWTRLPQGFKNSPTLFDEALNRDLQGFRLDHPSVSLLQYVDDLLIAADTQAACLSATRDLLMTLAELGYRVSGKKAQLCQEEVTYLGFKIHKGSRSLSNSRTQAILQIPVPKTKRQVREFLGTIGYCRLWIPGFAELAQPLYAATRGGNDPLVWGEKEEEAFQSLKLALTQPPALALPSLDKPFQLFVEETSGAAKGVLTQALGPWKRPVAYLSKRLDPVAAGWPRCLRAIAAAALLTREASKLTFGQDIEITSSHNLESLLRSPPDKWLTNARITQYQVLLLDPPRVRFKQTAALNPATLLPETDDTLPIHHCLDTLDSLTSTRPDLTDQPLAQAEATLFTDGSSYIRDGKRYAGAAVVTLDSVIWAEPLPIGTSAQKAELIALTKALEWSKDKSVNIYTDSRYAFATLHVHGMIYRERGLLTAGGKAIKNAPEILALLTAVWLPKRVAVMHCKGHQKDDAPTSTGNRRADEVAREVAIRPLSTQATISDAPDMPDTETPQYSNVEEALG

SEQ ID NO: 56 - protein, 689 aa, unidentified
  source 1..689: note = BABOON ENDOGENOUS VIRUS
VSLQDEHRLFDIPVTTSLPDVWLQDFPQAWAETGGLGRAKCQAPIIIDLKPTAVPVSIKQYPMSLEAHMGIRQHIIKFLELGVLRPCRSPWNTPLLPVKKPGTQDYRPVQDLREINKRTVDIHPTVPNPYNLLSTLKPDYSWYTVLDLKDAFFCLPLAPQSQELFAFEWKDPERGISGQLTWTRLPQGFKNSPTLFDEALHRDLTDFRTQHPEVTLLQYVDDLLLAAPTKKACTQGTRHLLQELGEKGYRASAKKAQICQTKVTYLGYILSEGKRWLTPGRIETVARIPPPRNPREVREFLGTAGFCRLWIPGFAELAAPLYALTKESTPFTWQTEHQLAFEALKKALLSAPALGLPDTSKPFTLFLDERQGIAKGVLTQKLGPWKRPVAYLSKKLDPVAAGWPPCLRIMAATAMLVKDSAKLTLGQPLTVITPHTLEAIVRQPPDRWITNARLTHYQALLLDTDRVQFGPPVTLNPATLLPVPENQPSPHDCRQVLAETHGTREDLKDQELPDADHTWYTDGSSYLDSGTRRAGAAVVDGHNTIWAQSLPPGTSAQKAELIALTKALELSKGKKANIYTDSRYAFATAHTHGSIYERRGLLTSEGKEIKNKAEIIALLKALFLPQEVAIIHCPGHQKGQDPVAVGNRQADRVARQAAMAEVLTLATEPDNTSHITIEHTYTSEDQEEA

SEQ ID NO: 57 - protein, 678 aa, unidentified
  source 1..678: note = GIBBON APE LEUKEMIA VIRUS
LNLEEEYRLHEKPVPSSIDPSWLQLFPTVWAERAGMGLANQVPPVVVELRSGASPVAVRQYPMSKEAREGIRPHIQKFLDLGVLVPCRSPWNTPLLPVKKPGTNDYRPVQDLREINKRVQDIHPTVPNPYNLLSSLPPSYTWYSVLDLKDAFFCLRLHPNSQPLFAFEWKDPEKGNTGQLTWTRLPQGFKNSPTLFDEALHRDLAPFRALNPQVVLLQYVDDLLVAAPTYEDCKKGTQKLLQELSKLGYRVSAKKAQLCQREVTYLGYLLKEGKRWLTPARKATVMKIPVPTTPRQVREFLGTAGFCRLWIPGFASLAAPLYPLTKESIPFIWTEEHQQAFDHIKKALLSAPALALPDLTKPFTLYIDERAGVARGVLTQTLGPWRRPVAYLSKKLDPVASGWPTCLKAVAAVALLLKDADKLTLGQNVTVIASHSLESIVRQPPDRWMTNARMTHYQSLLLNERVSFAPPAVLNPATLLPVESEATPVHRCSEILAEETGTRRDLEDQPLPGVPTWYTDGSSFITEGKRRAGAPIVDGKRTVWASSLPEGTSAQKAELVALTQALRLAEGKNINIYTDSRYAFATAHIHGAIYKQRGLLTSAGKDIKNKEEILALLEAIHLPRRVAIIHCPGHQRGSNPVATGNRRADEAAKQAALSTRVLAGTTKPQEPIEPAQEK

SEQ ID NO: 58 - protein, 678 aa, unidentified
  source 1..678: note = KOALA RETROVIRUS
MNLEEEYRLHEKPVPPSIDPSWLQLFPMVWAEKAGMGLANQVPPVVVELKSDASPVAVRQYPMSKEAREGIRPHIQRFLDLGILVPCQSPWNTPLLPVKKPGTNDYRPVQDLREVNKRVQDIHPTVPNPYNLLSSLPPSHTWYSVLDLKDAFFCLKLHPNSQPLFAFEWRDPEKGNTGQLTWTRLPQGFKNSPTLFDEALHRDLASFRALNPQVVMLQYVDDLLVAAPTYRDCKEGTRRLLQELSKLGYRVSAKKAQLCREEVTYLGYLLKGGKRWLTPARKATVMKIPTPTTPRQVREFLGTAGFCRLWIPGFASLAAPLYPLTREKVPFTWTEAHQEAFGRIKEALLSAPALALPDLTKPFALYVDEKEGVARGVLTQTLGPWRRPVAYLSKKLDPVASGWPTCLKAIAAVALLLKDADKLTLGQNVLVIAPHNLESIVRQPPDRWMTNARMTHYQSLLLNERVSFAPPAILNPATLLPVESDDTPIHICSEILAEETGTRPDLRDQPLPGVPAWYTDGSSFIMDGRRQAGAAIVDNKRTVWASNLPEGTSAQKAELIALTQALRLAEGKSINIYTDSRYAFATAHVHGAIYKQRGLLTSAGKDIKNKEEILALLEAIHLPKRVAIIHCPGHQRGTDPVATGNRKADEAAKQAAQSTRILTETTKNQEHFEPTRGK

SEQ ID NO: 59 - protein, 641 aa, unidentified
  source 1..641: note = MASON-PFIZER MONKEY VIRUS
MWGRDLLSQMKIMMCSPNDIVTAQMLAQGYSPGKGLGKKENGILHPIPNQGQSNKKGFGNFLTAAIDILAPQQCAEPITWKSDEPVWVDQWPLTNDKLAAAQQLVQEQLEAGHITESSSPWNTPIFVIKKKSGKWRLLQDLRAVNATMVLMGALQPGLPSPVAIPQGYLKIIIDLKDCFFSIPLHPSDQKRFAFSLPSTNFKEPMQRFQWKVLPQGMANSPTLCQKYVATAIHKVRHAWKQMYIIHYMDDILIAGKDGQQVLQCFDQLKQELTAAGLHIAPEKVQLQDPYTYLGFELNGPKITNQKAVIRKDKLQTLNDFQKLLGDINWLRPYLKLTTGDLKPLFDTLKGDSDPNSHRSLSKEALASLEKVETAIAEQFVTHINYSLPLIFLIFNTALTPTGLFWQDNPIMWIHLPASPKKVLLPYYDAIADLIILGRDHSKKYFGIEPSTIIQPYSKSQIDWLMQNTEMWPIACASFVGILDNHYPPNKLIQFCKLHTFVFPQIISKTPLNNALLVFTDGSSTGMAAYTLTDTTIKFQTNLNSAQLVELQALIAVLSAFPNQPLNIYTDSAYLAHSIPLLETVAQIKHISETAKLFLQCQQLIYNRSIPFYIGHVRAHSGLPGPIAQGNQRADLATKIVA

SEQ ID NO: 60 - protein, 571 aa, unidentified
  source 1..571: note = POK11ERV
ATVEPPKPIPLTWKTEKPVWVNQWPLPKQKLEALHLLANEQLEKGHIEPSFSPWNSPVFVIQKKSGKWRMLTDLRAVNAVIQPMGPLQPGLPSPAMIPKDWPLIIIDLKDCFFTIPLAEQDCEKFAFTIPAINNKEPATRFQWKVLPQGMLNSPTICQTFVGRALQPVREKFSDCYIIHYIDDILCAAETKDKLIDCYTFLQAEVANAGLAIASDKIQTSTPFHYLGMQIENRKIKPQKIEIRKDTLKTLNDFQKLLGDINWIRPTLGIPTYAMSNLFSILRGDSDLNSKRILTPEATKEIKLVEEKIQSAQINRIDPLAPLQLLIFATAHSPTGIIIQNTDLVEWSFLPHSTVKTFTLYLDQIATLIGQTRLRIIKLCGNDPDKIVVPLTKEQVRQAFINSGAWQIGLANFVGIIDNHYPKTKIFQFLKMTTWILPKITRREPLENALTVFTDGSSNGKAAYTGPKERVIKTPYQSAQRAELVAVITVLQDFDQPINIISDSAYVVQATRDVETALIKYSMDDQLNQLFNLLQQTVRKRNFPFYITHIRAHTNLPGPLTKANEEADLLVS

SEQ ID NO: 61 - protein, 641 aa, unidentified
  source 1..641: note = SIMIAN RETROVIRUS TYPE 2
MWGRDLLSQMKIMMCSPNDIVTAQMLAQGYSPGKGLGKREDGILQPIPNSGQLDRKGFGNFLATAVDILAPQRYADPITWKSDEPVWVDQWPLTQEKLAAAQQLVQEQLQAGHIIESNSPWNTPIFVIKKKSGKWRLLQDLRAVNATMVLMGALQPGLPSPVAIPQGYFKIVIDLKDCFFTIPLQPVDQKRFAFSLPSTNFKQPMKRYQWKVLPQGMANSPTLCQKYVAAAIEPVRKSWAQMYIIHYMDDILIAGKLGEQVLQCFAQLKQALTTTGLQIAPEKVQLQDPYTYLGFQINGPKITNQKAVIRRDKLQTLNDFQKLLGDINWLRPYLHLTTGDLKPLFDILKGDSNPNSPRSLSEAALASLQKVETAIAEQFVTQIDYTQPLTFLIFNTTLTPTGLFWQNNPVMWVHLPASPKKVLLPYYDAIADLIILGRDNSKKYFGLEPSTIIQPYSKSQIHWLMQNTETWPIACASYAGNIDNHYPPNKLIQFCKLHAVVFPRIISKTPLDNALLVFTDGSSTGIAAYTFEKTTVRFKTSHTSAQLVELQALIAVLSAFPHRALNVYTDSAYLAHSIPLLETVSHIKHISDTAKFFLQCQQLIYNRSIPFYLGHIRAHSGLPGPLSQGNHITDLATKVVA

SEQ ID NO: 62 - protein, 672 aa, unidentified
  source 1..672: note = WOOLLY MONKEY SARCOMA VIRUS
LNLEEEYRLHEKPVPSSIDPSWLQLFPTVWAERAGMGLANQVPPVVVELRSGASPVAVRQYPMSKEAREGIRPHIQRFLDLGVLVPCQSPWNTPLLPVKKPGTNDYRPVQDLREINKRVQDIHPTVPNPYNLLSSLPPSHTWYSVLDLKDAFFCLKLHPNSQPLFAFEWRDPEKGNTGQLTWTRLPQGFKNSPTLFDEALHRDLAPFRALNPQVVLLQYVDDLLVAAPTYRDCKEGTQKLLQELSKLGYRVSAKKAQLCQKEVTYLGYLLKEGKRWLTPARKATVMKIPPPTTPRQVREFLGTAGFCRLWIPGFASLAAPLYPLTKESIPFIWTEEHQKAFDRIKEALLSAPALALPDLTKPFTLYVDERAGVARGVLTQTLGPWRRPVAYLSKKLDPVASGWPTCLKAVAAVALLLKDADKLTLGQNVTVIASHSLESIVRQPPDRWMTNARMTHYQSLLLNERVSFAPPAVLNPATLLPVESEATPVHRCSEILAEETGTRRDLKDQPLPGVPAWYTDGSSFIAEGKRRAGAAIVDGKRTVWASSLPEGTSAQKAELVALTQALRLAEGKDINIYTDSRYAFATAHIHGAIYKQRGLLTSAGKDIKNKEEILALLEAIHLPKRVAIIHCPGHQKGNDPVATGNRRADEAAKQAALSTRVLAETTKPQELI

SEQ ID NO: 63 - protein, 292 aa, Synthetic construct
NSQAQSACCAGANQIVEGATLEKVVAPACLQQAWTRVRKNKGGPGGDGVTIEIFAQNAEVELEKLRAETLAGIYRPRKVRHAIVPKPKGGERKLTIPSVVDRILQTATMLSLGQTVDHHFSSASWAYREGRGVDDALADLRRLRNSGLFWTFDADIMQYFDRILHKRLIDDLFIWVDDLRIVRLIQLWLRSFSYWGRGIAQGAPISPLLANLFLHPMDRLLELEGLASVRYADDFVVLCRSKALAQKAQLIVASHLAARGLKLNMSKTRILAPSEAFIFLGQTVEPVWDTQP

SEQ ID NO: 64 - protein, 312 aa, Synthetic construct
NLVKRLAHHLGKSEPEVIHFLADAPNKYRVYKIPKRSYGHRVIAQPTRELKLYQKAFLELYSFPVHSSATAYCKGKSIKDNALSHVKNHYLLKTDLENFFNSITPNIFWKSIENDSIATPKFSTSEIALVERLIFWRPSKLQGGKLVLSVGAPSSPTISNFCLYQFDEYLSIICKEQNISYTRYADDLTFSTCDKDVLHTVIPLIQSLLDYFFASELKLNHSKTVFSSKAHNRHVTGITLNNEGKLSLGRERKRYIKHLVHSFKYGKLDNTEIRHLQGMLSFAKHIEPIFIDRLKEKYTDELIKIIYEAGHE

SEQ ID NO: 65 - protein, 314 aa, Synthetic construct
NILTTLREQLLTNNVIMPQEFERLEVRGSHAYKVYSIPKRKAGRRTIAHPSSKLKICQRHLNAILNPLLKVHDSSYAYVKGRSIKDNALVHSHSAYVLKMDFQNFFNSITPTILRQCLIQNDILLSVNELEKLEQLIFWNPSKKRNGKLILSVGSPISPLISNAIMYPFDKIINDICTKHGINYTRYADDITFSTNIKNTLNKLPEIVEQLIIQTYAGRIIINKRKTVFSSKKHNRHVTGITLTNDSKISIGRSRKRYISSLVFKYINKNLDIDEINHMKGMLAFAYNIEPIYIHRLSHKYKVNIVEKILRGSN

SEQ ID NO: 66 - protein, 419 aa, Synthetic construct
ALLERILARDNLITALKRVEANQGAPGIDGVSTDQLRDYIRAHWSTIHAQLLAGTYRPAPVRRVEIPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQRYFELRQG

SEQ ID NO: 67 - protein, 426 aa, Synthetic construct
DTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTC

SEQ ID NO: 68 - protein, 513 aa, Synthetic construct
AGQPTSREALYERIRSTSKEEVILEEMIRLGFWPAQGAVPHDPAEEIRRRGELERQLSELREKSRKLYNEKALIAEQRKQRLAESRRKQKETKARRERERQERAQKWAQRKAGEILFLGEDVSGGMSHKTCDAELIKREGVPAIASAEELARAMGIALKELRFLAYNRKVSRVTHYRRFLLPKKTGGLRLISAPMPRLKRAQAWALEHIFNKLSFEPAAHGFVAGRSIVSNARPHVGADVVVNLDLKDFFPTVSFPRVKGALRHLGYSESVATALALVCTEPEVDEVGLDGTTWYVARGERFLPQGSPCSPAITNLLCRRLDRRLHGLAQALGFVYTRYADDLTFSGRGEAAESKRVGKLLRGAADIVAHEGFVVHPDKTRVMRRGRRQEVTGVVVNDKTSVPRDELRKFRATLYQIEKDGPADKRWGNGGDVLAAVHGYACFVAMVDPSRGQPLLARARALLAKHGGPSKPPGGSGPRAPTPVQPTANAPEAPKPVAPATPAAPAKKGWKLF

SEQ ID NO: 69 - DNA, 110 bp, Synthetic construct
  source 1..110: mol_type = other DNA
ctgatgtgttcgttgcaccgactcggtgccactttttcaagttgataacggactagccttattttaacttgctatttctagctctaaaaccaggtaatgactaagatgac

SEQ ID NO: 70 - DNA, 108 bp, Synthetic construct
  source 1..108: mol_type = other DNA
tggcgtctacatgcaccgactcggtgccactttttcaagttgataacggactagccttattttaacttgctatttctagctctaaaacccaaggccccccaccactgc

SEQ ID NO: 71 - DNA, 113 bp, Synthetic construct
  source 1..113: mol_type = other DNA
accttgatcgcttttccgcaccgactcggtgccactttttcaagttgataacggactagccttattttaacttgctatttctagctctaaaacggtgctgcagaagggattcc

SEQ ID NO: 72 - DNA, 112 bp, Synthetic construct
  source 1..112: mol_type = other DNA
gaagtgctcccatcacgcaccgactcggtgccactttttcaagttgataacggactagccttattttaacttgctatttctagctctaaaacttcttcttctgctcggactc

SEQ ID NO: 73 - DNA, 118 bp, Synthetic construct
  source 1..118: mol_type = other DNA
tgagtgctccagatggcacattgcaccgactcggtgccactttttcaagttgataacggactagccttattttaacttgctatttctagctctaaaactcatctggcctgcagacatc

SEQ ID NO: 74 - DNA, 110 bp, Synthetic construct
  source 1..110: mol_type = other DNA
tcaggaaaggagcgcaccgactcggtgccactttttcaagttgataacggactagccttattttaacttgctatttctagctctaaaactgagtggcaagggcaaccctc

SEQ ID NO: 75 - DNA, 10 bp, Synthetic construct
  source 1..10: mol_type = other DNA
ggcccagact

SEQ ID NO: 76 - DNA, 10 bp, Synthetic construct
  source 1..10: mol_type = other DNA
gattcctggt

SEQ ID NO: 77 - DNA, 10 bp, Synthetic construct
  source 1..10: mol_type = other DNA
gtcatcttag

SEQ ID NO: 78 - DNA, 10 bp, Synthetic construct
  source 1..10: mol_type = other DNA
gcattttcag

SEQ ID NO: 79 - protein, 34 aa, Synthetic construct
SGGSSGGSKRTADGSEFESPKKKRKVSGGSSGGS

SEQ ID NO: 80 - protein, 33 aa, Synthetic construct
SGGSSGGSSGSETPGTSESATPESSGGSSGGSS

SEQ ID NO: 81 - protein, 120 aa, Synthetic construct
  VARIANT 5..120: note = up to 29 copies of SGGS could be deleted
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 82 - protein, 4 aa, Synthetic construct
SGGS

SEQ ID NO: 83 - protein, 60 aa, Synthetic construct
SGGSSGGSSGSETPGTSESATPESAGSYPYDVPDYAGSAAPAAKKKKLDGSGSGGSSGGS

SEQ ID NO: 84 - protein, 150 aa, Synthetic construct
  VARIANT 6..150: note = up to 29 copies of GGGGS could be deleted
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 85 - protein, 30 aa, Synthetic construct
  VARIANT 2..30: note = up to 29 copies of G could be deleted
GGGGGGGGGGGGGGGGGGGGGGGGGGGGGG

SEQ ID NO: 86 - protein, 150 aa, Synthetic construct
  VARIANT 6..150: note = up to 29 copies of EAAAK could be deleted
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 87 - protein, 90 aa, Synthetic construct
  VARIANT 4..90: note = up to 29 copies of GGS could be deleted
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 88 - protein, 60 aa, Synthetic construct
  VARIANT 3..60: note = up to 29 copies of XP could be deleted
XPXPXPXPXPXPXPXPXPXPXPXPXPXPXPXPXPXPXPXPXPXPXPXPXPXPXPXPXPXP

SEQ ID NO: 89 - protein, 16 aa, Synthetic construct
SGSETPGTSESATPES

SEQ ID NO: 90 - protein, 32 aa, Synthetic construct
SGGSSGGSSGSETPGTSESATPESSGGSSGGS

SEQ ID NO: 91 - protein, 10 aa, Synthetic construct
SGGSGGSGGS

SEQ ID NO: 92 - protein, 6 aa, Synthetic construct
GGSGGS

SEQ ID NO: 93 - protein, 9 aa, Synthetic construct
GGSGGSGGS

SEQ ID NO: 94 - protein, 7 aa, Synthetic construct
PKKKRKV

SEQ ID NO: 95 - protein, 19 aa, Synthetic construct
MKRTADGSEFESPKKKRKV

SEQ ID NO: 96 - protein, 21 aa, Synthetic construct
SGGSKRTADGSEFEPKKKRKV

SEQ ID NO: 97 - protein, 18 aa, Synthetic construct
KRTADGSEFESPKKKRKV

SEQ ID NO: 98 - protein, 9 aa, Synthetic construct
PAAKRVKLD

SEQ ID NO: 99 - protein, 30 aa, Synthetic construct
MDSLLMNRRKFLYQFKNVRWAKGRRETYLC

SEQ ID NO: 100 - protein, 20 aa, Synthetic construct
AVKRPAATKKAGQAKKKKLD

SEQ ID NO: 101 - protein, 25 aa, Synthetic construct
MSRRRKANPTKLSENAKKLAKEVEN

SEQ ID NO: 102 - protein, 9 aa, Synthetic construct
KLKIKRPVK

SEQ ID NO: 103 - protein, 8 aa, Synthetic construct
VSRKRPRP

SEQ ID NO: 104 - protein, 10 aa, Synthetic construct
EGAPPAKRAR

SEQ ID NO: 105 - protein, 12 aa, Synthetic construct
PPQPKKKPLDGE

SEQ ID NO: 106 - protein, 17 aa, Synthetic construct
KRTADGSEFEPKKKRKV

SEQ ID NO: 107 - protein, 19 aa, Synthetic construct
NLSKRPAAIKKAGQAKKKK

SEQ ID NO: 108 - protein, 11 aa, Synthetic construct
RQRRNELKRSF

SEQ ID NO: 109 - protein, 38 aa, Synthetic construct
NQSSNFGPMKGGNFGGRSSGPYGGGGQYFAKPRNQGGY

SEQ ID NO: 110 - protein, 16 aa, unidentified
  source 1..16: note = Xenopus nucleoplasmin
KRXXXXXXXXXXKKKL

SEQ ID NO: 111 - DNA, 10 bp, Synthetic construct
  source 1..10: mol_type = other DNA
gatgtctgca

SEQ ID NO: 112 - DNA, 10 bp, Synthetic construct
  source 1..10: mol_type = other DNA
ggaatccctt

SEQ ID NO: 113 - DNA, 125 bp, Synthetic construct
  source 1..125: mol_type = other DNA
nnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 114 - DNA, 121 bp, Synthetic construct
  source 1..121: mol_type = other DNA
nnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 115 - DNA, 109 bp, Synthetic construct
  source 1..109: mol_type = other DNA
nnnnnnnnnnnnnnnnnnnngtttttgtactctcagaaatgcagaagctacaaagataaggcttcatgccgaaatcaacaccctgtcattttatggcagggtgtttttt

SEQ ID NO: 116 - DNA, 102 bp, Synthetic construct
  source 1..102: mol_type = other DNA
nnnnnnnnnnnnnnnnnnnngttttagagctagaaatagcaagttaaaataaggctagtccgttatcaacttgaaaaagtggcaccgagtcggtgctttttt

SEQ ID NO: 117 - DNA, 88 bp, Synthetic construct
  source 1..88: mol_type = other DNA
nnnnnnnnnnnnnnnnnnnngttttagagctagaaatagcaagttaaaataaggctagtccgttatcaacttgaaaaagtgttttttt

SEQ ID NO: 118 - DNA, 76 bp, Synthetic construct
  source 1..76: mol_type = other DNA
nnnnnnnnnnnnnnnnnnnngttttagagctagaaatagcaagttaaaataaggctagtccgttatcatttttttt

SEQ ID NO: 119 - RNA, 82 nt, Synthetic construct
  source 1..82: mol_type = other RNA
gttttagagctagaaatagcaagttaaaataaaggctagtccgttatcaacttgaaaaagtggcaccgagtcggtgcttttt

SEQ ID NO: 120 - DNA, 10 bp, Synthetic construct
  source 1..10: mol_type = other DNA
gagtccgagc

SEQ ID NO: 121 - DNA, 10 bp, Synthetic construct
  source 1..10: mol_type = other DNA
gtatggaaaa

SEQ ID NO: 122 - DNA, 58 bp, Synthetic construct
  source 1..58: mol_type = other DNA
gattacaaggatgacgacgataagtgatggcagaggaaaggaagccctgcttcctcca

SEQ ID NO: 123 - DNA, 14 bp, Synthetic construct
  source 1..14: mol_type = other DNA
ctgatgtgttcgtt

SEQ ID NO: 124 - DNA, 96 bp, Synthetic construct
  source 1..96: mol_type = other DNA
gcaccgactcggtgccactttttcaagttgataacggactagccttattttaacttgctatttctagctctaaaaccaggtaatgactaagatgac

SEQ ID NO: 125 - DNA, 46 bp, Synthetic construct
  source 1..46: mol_type = other DNA
gattacaaggatgacgacgataagtgagtgctccagatggcacatt

SEQ ID NO: 126 - DNA, 96 bp, Synthetic construct
  source 1..96: mol_type = other DNA
gcaccgactcggtgccactttttcaagttgataacggactagccttattttaacttgctatttctagctctaaaactcatctggcctgcagacatc

SEQ ID NO: 127 - DNA, 11 bp, Synthetic construct
  source 1..11: mol_type = other DNA
acagtggtgac

SEQ ID NO: 128 - DNA, 97 bp, Synthetic construct
  source 1..97: mol_type = other DNA
gcaccgactcggtgccactttttcaagttgataacggactagccttattttaacttgctatttctagctctaaaacgcgcgaacagctccagcccgc

SEQ ID NO: 129 - DNA, 17 bp, Synthetic construct
  source 1..17: mol_type = other DNA
accttgatcgcttttcc

SEQ ID NO: 130 - DNA, 96 bp, Synthetic construct
  source 1..96: mol_type = other DNA
gcaccgactcggtgccactttttcaagttgataacggactagccttattttaacttgctatttctagctctaaaacggtgctgcagaagggattcc

SEQ ID NO: 131 - DNA, 16 bp, Synthetic construct
  source 1..16: mol_type = other DNA
gaagtgctcccatcac

SEQ ID NO: 132 - DNA, 96 bp, Synthetic construct
  source 1..96: mol_type = other DNA
gcaccgactcggtgccactttttcaagttgataacggactagccttattttaacttgctatttctagctctaaaacttcttcttctgctcggactc

SEQ ID NO: 133 - protein, 1368 aa, Escherichia coli
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLKREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGD

SEQ ID NO: 134 - DNA, 40 bp, Synthetic construct
  source 1..40: mol_type = other DNA
ttagctcgcgaacagttggcccttggcatgagtcttacga

SEQ ID NO: 135 - DNA, 41 bp, Synthetic construct
  source 1..41: mol_type = other DNA
ttagctcgcgaacagttggcccttgagcatgagtcttacga

SEQ ID NO: 136 - DNA, 40 bp, Synthetic construct
  source 1..40: mol_type = other DNA
gaagcaatgaactggcttaagtctggaggtcaaagataag

SEQ ID NO: 137 - DNA, 61 bp, Synthetic construct
  source 1..61: mol_type = other DNA
gaagcaatgaactggcttaagtctgctgctaagcttgctggctgctgaggtcaaagataag

SEQ ID NO: 138 - DNA, 51 bp, Synthetic construct
  source 1..51: mol_type = other DNA
tggaggaagcagggcttcctttcctctgccatcaaagccctgcttcctcca

SEQ ID NO: 139 - DNA, 51 bp, Synthetic construct
  source 1..51: mol_type = other DNA
tggaggaagcagggcttcctttcctctgccatcaaagcactggatcctcca

SEQ ID NO: 140 - DNA, 27 bp, Synthetic construct
  source 1..27: mol_type = other DNA
aagccgaactctttggactgaacgctt

SEQ ID NO: 141 - DNA, 47 bp, Synthetic construct
  source 1..47: mol_type = other DNA
aagccgaactccttactcccgtaagccagcgtcatgactgaacgctt

SEQ ID NO: 142 - DNA, 40 bp, Synthetic construct
  source 1..40: mol_type = other DNA
ctctcgttgtccaggtatggcccgggtccactgccttccc

SEQ ID NO: 143 - DNA, 40 bp, Synthetic construct
  source 1..40: mol_type = other DNA
ctctcgttgtccaggtatggcccggctccactgccttccc

SEQ ID NO: 144 - DNA, 40 bp, Synthetic construct
  source 1..40: mol_type = other DNA
ctctcgttgtccaggtatggcccgggtccacttcaccaac

SEQ ID NO: 145 - protein, 1368 aa, Synthetic construct
MDKKYSIGLDIGTNSVGWAVITGEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRRKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 146 - protein, 19 aa, Synthetic construct
  SITE 19: note = This sequence is linked C-terminally to a Cas9 protein
MKRTADGSEFESPKKKRKV

SEQ ID NO: 147 - protein, 566 aa, Synthetic construct
  SITE 1: note = This sequence is linked N-terminally to a Cas9 protein
SGGSSGGSKRTADGSEFESPKKKRKVSGGSSGGSISSSKHTLSQMNKVSNIVKEPELPDIYKEFKDITADTNTEKLPKPIKGLEFEVELTQENYRLPIRNYPLTPVKMQAMNDEINQGLKGGIIRESKAINACPVIFVPRKEGTLRMVVDYRPLNKYVKPNVYPLPLIEQLLAKIQGSTIFTKLDLKSAYHQIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGLTPCQENIDKVLQWKQPKNRKELRQFLGSVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISIKRTADGSEFESPKKKRKVPAAKRVKLD

SEQ ID NO: 148 - protein, 566 aa, Synthetic construct
  SITE 1: note = This sequence is linked N-terminally to a Cas9 protein
SGGSSGGSKRTADGSEFESPKKKRKVSGGSSGGSISSSKHTLSQMNKVSNIVKEPELPDIYKEFKDITADTNTEKLPKPIKGLEFEVELTQENYRLPIRNYPLTPVKMQAMNDEINQGLKGGIIRESKAINACPVIFVPRKEGTLRMVVDYRPLNKYVKPNVYPLPLIEQLLAKIQGSTIFTKLDLKSAYHQIRVRKGDEHKLAFRCPRGVFEYLVMPYGIKTAPAHFQYFINTILGEAKESHVVCYMDDILIHSKSESEHVKHVKDVLQKLKNANLIINQAKCEFHQSQVKFLGYHISEKGLTPCQENIDKVLQWKQPKNQKELRQFLGQVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISIKRTADGSEFESPKKKRKVPAAKRVKLD

SEQ ID NO: 149 - protein, 558 aa, Synthetic construct
  SITE 1: note = This sequence is linked N-terminally to a Cas9 protein
SGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPNVPNPYNLLSGLPPSHQWYTVLDLKDAFFCLRLHPTSQPLFAFEWRDPEMGISGQLTWTRLPQGFKNSPTLFCEALHRDLADFRIQHPDLILLQYYDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDKRTADGSEFESPKKKRKVPAAKRVKLD

SEQ ID NO: 150 - protein, 1420 aa, Synthetic construct
  SITE 1420: note = This sequence is linked C-terminally to a reverse transcriptase protein
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQRNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIARQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGS

SEQ ID NO: 151 - protein, 27 aa, Synthetic construct
  SITE 1: note = This sequence is linked N-terminally to a reverse transcriptase protein
KRTADGSEFESPKKKRKVPAAKRVKLD

SEQ ID NO: 152 - protein, 1420 aa, Synthetic construct
  SITE 1420: note = This sequence is linked C-terminally to a reverse transcriptase protein
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFRRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEKTITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMVEERLKTYAHLFDNKVMKQLKRRRYTGWGRLSRKLINGIRDKQSGKTILDFLKSDGFANRNFMQLIHDDSLTFKEDIQKAQVSGQGDSLYEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIARQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGS

SEQ ID NO: 153 - protein, 1420 aa, Synthetic construct
  SITE 1420: note = This sequence is linked C-terminally to a reverse transcriptase protein
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFRRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEKTITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMVEERLKTYAHLFDNKVMKQLKRCRYTGWGRLSRKLINGIRDKQSGKTILDFLKSDGFANRNFMQLIHDDSLTFKEDIQKAQVSGQGDSLYEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGS

SEQ ID NO: 154 - protein, 468 aa, Synthetic construct
  SITE 1: note = This sequence is linked N-terminally to a Cas9 protein
SGGSSGGSKRTADGSEFESPKKKRKVSGGSSGGSGRPYVTLNLNGMFMDKFKPYSKSNAPITTLEKLSKALSISVEELKAIAELSLDEKYTLKKIPKIDGSKRIVYSLHPKMRLLQSRINERIFKELVVFPSFLFGSVPSKNDVLNSNVKRDYVSCAKAHCGAKTVLKVDISNFFDNIHRDLVRSVFEEILHIKDEALDYLVDICTKDDFVVQGALTSSYIATLCLFAVEGDVVRRAQRKGLVYTRLVDDITVSSKISNYDFSQMQSHIERMLSEHNLPINKHKTKIFHCSSEPIKVHGLIVDYDSPRLPSDKVKRIRASIHNLKLLAAKNNTKTSVAYRKEFNRCMGRVNELGRVGHEKYESFKKQLQAIKPMPSNRDVAVIDAAIKSLELSYSKGNQNKHWYKRKYDLTRYKMIILTRSESFKEKLECFKSRLASLKPLKRTADGSEFESPKKKRKVPAAKRVKLD

SEQ ID NO: 155 - protein, 1952 aa, Synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLKREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGDSGGSSGGSKRTADGSEFESPKKKRKVSGGSSGGSISSSKHTLSQMNKVSNIVKEPELPDIYKEFKDITADTNTEKLPKPIKGLEFEVELTQENYRLPIRNYPLTPVKMQAMNDEINQGLKGGIIRESKAINACPVIFVPRKEGTLRMVVDYRPLNKYVKPNVYPLPLIEQLLAKIQGSTIFTKLDLKSAYHQIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGLTPCQENIDKVLQWKQPKNRKELRQFLGSVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISIKRTADGSEFESPKKKRKVPAAKRVKLD

SEQ ID NO: 156 - protein, 1952 aa, Synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLKREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGDSGGSSGGSKRTADGSEFESPKKKRKVSGGSSGGSISSSKHTLSQMNKVSNIVKEPELPDIYKEFKDITADTNTEKLPKPIKGLEFEVELTQENYRLPIRNYPLTPVKMQAMNDEINQGLKGGIIRESKAINACPVIFVPRKEGTLRMVVDYRPLNKYVKPNVYPLPLIEQLLAKIQGSTIFTKLDLKSAYHQIRVRKGDEHKLAFRCPRGVFEYLVMPYGIKTAPAHFQYFINTILGEAKESHVVCYMDDILIHSKSESEHVKHVKDVLQKLKNANLIINQAKCEFHQSQVKFLGYHISEKGLTPCQENIDKVLQWKQPKNQKELRQFLGQVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISIKRTADGSEFESPKKKRKVPAAKRVKLD

SEQ ID NO: 157 - protein, 1948 aa, Synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLKREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPNVPNPYNLLSGLPPSHQWYTVLDLKDAFFCLRLHPTSQPLFAFEWRDPEMGISGQLTWTRLPQGFKNSPTLFCEALHRDLADFRIQHPDLILLQYYDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDSGGSKRTADGSEFESPKKKRKVPAAKRVKLD

SEQ ID NO: 158 - protein, 2128 aa, Synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLKREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPFVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYMDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVPAAKRVKLD